(19) United States
(12) Patent Application Publication
(10) Pub. No.: US 2025/0289802 A1
(43) Pub. Date: Sep. 18, 2025

(54) 3-(1-OXO-5-(PIPERIDIN-4-YL)ISOINDOLIN-2-YL)PIPERIDINE-2,6-DIONE DERIVATIVES AND USES THEREOF

(71) Applicant: NOVARTIS AG, Basel (CH)

(72) Inventors: Rohan Eric John BECKWITH, San Diego, CA (US); Simone BONAZZI, Cambridge, MA (US); Artiom CERNIJENKO, Cambridge, MA (US); Michael Scott VISSER, Braintree, MA (US)

(21) Appl. No.: 19/052,946

(22) Filed: Feb. 13, 2025

Related U.S. Application Data
(63) Continuation of application No. 17/430,473, filed on Aug. 12, 2021, now abandoned.
(60) Provisional application No. 62/806,140, filed on Feb. 15, 2019.

(51) Int. Cl. C07D 401/14; A61K 31/4545; A61K 31/497; A61K 31/501; A61K 31/506; A61P 35/00

(52) U.S. Cl. CPC C07D 401/14; A61K 31/4545; A61K 31/497; A61K 31/501; A61K 31/506; A61P 35/00

(57) ABSTRACT

The present disclosure provides a compound of Formula (I):

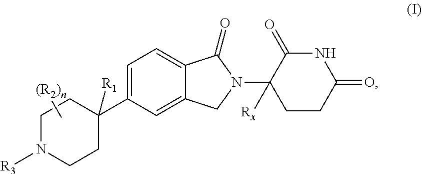

or a pharmaceutically acceptable salt hydrate, solvate, prodrug, stereoisomer, or tautomer thereof, wherein R₁, R₂, R₃, Rₓ, and n are as defined herein, and methods of making and using same.

### RELATED APPLICATIONS

This application is a continuation of U.S. patent application Ser. No. 17/430,473, filed Aug. 12, 2021, which is a U.S. National Phase Application, filed under 35 U.S.C. § 371, of International Application No. PCT/IB2020/051205, filed Feb. 13, 2020, which claims benefit of and priority to U.S. Provisional Application No. 62/806,140, filed Feb. 15, 2019, the entire contents of which are incorporated herein by reference in its entirety.

### SEQUENCE LISTING

The instant application contains a Sequence Listing which has been submitted electronically in ASCII format and is hereby incorporated by reference in its entirety. Said ASCII copy, created on Feb. 13, 2025, is named PAT058462-US-CNT SL.html and is 473,546 bytes in size.

### FIELD OF THE DISCLOSURE

The present disclosure relates to 3-(1-oxo-5-(piperidin-4-yl)isoindolin-2-yl)piperidine-2,6-dione compounds and compositions and their use for the treatment of IKAROS Family Zinc Finger 2 (IKZF2)-dependent diseases or disorders or where reduction of IKZF2 or IKZF4 protein levels can ameliorate a disease or disorder.

### BACKGROUND OF THE DISCLOSURE

IKAROS Family Zinc Finger 2 (IKZF2) (also known as Helios) is one of the five members of the Ikaros family of transcription factors found in mammals. IKZF2 contains four zinc finger domains near the N-terminus, which are involved in DNA binding, and two zinc finger domains at the C-terminus, which are involved in protein dimerization. IKZF2 is about 50% identical with Ikaros family members, Ikaros (IKZF1), Aiolos (IKZF3), and Eos (IKZF4) with highest homology in the zinc finger regions (80%+ identity). These four Ikaros family transcription factors bind to the same DNA consensus site and can heterodimerize with each other when co-expressed in cells. The fifth Ikaros family protein, Pegasus (IKZF5), is only 25% identical to IKZF2, binds a different DNA site than other Ikaros family members and does not readily heterodimerize with the other Ikaros family proteins. IKZF2, IKZF1 and IKZF3 are expressed mainly in hematopoietic cells while IKZF4 and IKZF5 are expressed in a wide variety of tissues. (John, L. B., et al., (2011), Mol. Immunol. 48:1272-1278; Perdomo, J., et al., (2000), J. Biol. Chem. 275:38347-38354.) IKZF2 is believed to have an important role in the function and stability of regulatory T cells (Tregs). IKZF2 is highly expressed at the mRNA and protein level by regulatory T-cell populations. Knockdown of IKZF2 by siRNA has been shown to result in downregulation of FoxP3 and to impair the ability of isolated human CD4+CD25+ Tregs to block T-cell activation in vitro. Moreover, overexpression of IKZF2 in isolated murine Tregs has been shown to increase expression of Treg related markers such as CD103 and GITR and the IKZF2 overexpressing cells showed increased suppression of responder T-cells. IKZF2 has also been found to bind the promoter of FoxP3, the defining transcription factor of the regulatory T-cell lineage, and to affect FoxP3 expression.

Knockout of IKZF2 within FoxP3-expressing Tregs in mice has been shown to cause activated Tregs to lose their inhibitory properties, to express T-effector cytokines, and to take on T-effector functions. IKZF2 knockout mutant mice develop autoimmune disease by 6-8 months of age, with increased numbers of activated CD4 and CD8 T cells, follicular helper T cells and germinal center B cells. This observed effect is believed to be cell intrinsic, as Rag2−/− mice given bone marrow from IKZF2 knockout mice, but not bone marrow from IKZF2+/+ develop autoimmune disease. Direct evidence that IKZF2 affects regulatory T-cell function has been shown in the analysis of mice in which IKZF2 was deleted only in FoxP3 expressing cells (FoxP3-YFP-Cre Heliosfl/fl). The results showed that the mice also develop autoimmune disease with similar features as observed in the whole animal IKZF2 knockout. Moreover, pathway analysis of a CHIP-SEQ experiment has also suggested that IKZF2 is affecting expression of genes in the STAT5/IL-2Rα pathway in regulatory T-cells. This effect of IKZF2 loss was shown to be more apparent after an immune challenge (viral infection or injection with sheep's blood), and it was noted that after immune stimulation, the IKZF2 negative regulatory T cells began to take on features of effector T cells. (Getnet, D., et al., Mol. Immunol. (2010), 47:1595-1600; Bin Dhuban, K., et al., (2015), J. Immunol. 194:3687-96; Kim, H-J., et al., (2015), Science 350:334-339; Nakawaga, H., et al., (2016) PNAS, 113: 6248-6253)

Overexpression of Ikaros isoforms which lack the DNA binding regions have been shown to be associated with multiple human haematological malignancies. Recently, mutations in the IKZF2 gene, which lead to abnormal splicing variants, have been identified in adult T-cell leukemias and low hypodiploid acute lymphoblastic leukemia. It has been proposed that these isoforms, which are capable of dimerization, have a dominant negative effect on Ikaros family transcription factors which primes the development of lymphomas. IKZF2 knockout mutants that survive into adulthood do not develop lymphomas, supporting this hypothesis (Asanuma, S., et al., (2013), Cancer Sci. 104:1097-1106; Zhang, Z., et al., (2007), Blood 109:2190-2197; Kataoka, D., et al., (2015), Nature Genetics 47:1304-1315.)

Currently, anti-CTLA4 antibodies are used in the clinic to target Tregs in tumors. However, targeting CTLA4 often causes systemic activation of T-effector cells, resulting in excessive toxicity and limiting therapeutic utility. Up to 3/4 of patients treated with a combination of anti-PD1 and anti-CTLA4 have reported grade 3 or higher adverse events. Thus, a strong need exists to provide compounds that target Tregs in tumors without causing systemic activation of T-effector cells.

An IKZF2-specific degrader has the potential to focus the enhanced immune response to areas within or near tumors providing a potentially more tolerable and less toxic therapeutic agent for the treatment of cancer.

### SUMMARY OF THE DISCLOSURE

The compounds of the disclosure have use as therapeutic agents, particularly for cancers and related diseases. In one aspect, the compounds of the disclosure have IKZF2 degrader activity, preferably having such activity at or below the 50 μM level, and more preferably having such activity at or below the 10 μM level. In another aspect, the compounds of the disclosure have degrader activity for IKZF2 that is selective over one or more of IKZF1, IKZF3, IKZF4, and/or IKZF5. In another aspect, the compounds of the disclosure have degrader activity for both IKZF2 and IKZF4. The compounds of the disclosure have usefulness in treating cancer and other diseases for which such degrader activity would be beneficial for the patient. For example, while not intending to be bound by any theory, the inventors believe that reducing levels of IKZF2 in Tregs in a tumor may allow the patient immune system to more effectively attack the disease. In summary, the present disclosure provides novel IKZF2 degraders useful for the treatment of cancer and other diseases.

A first aspect of the present disclosure relates to compounds of Formula (I)

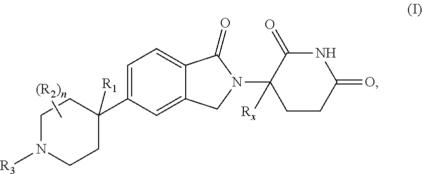

wherein:
   R₁ is (C₁-C₆)alkyl, (C₁-C₆)alkoxy, (C₁-C₆)haloalkyl, (C₁-C₆)haloalkoxy, (C₁-C₆)hydroxyalkyl, halogen,
—OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN;
each R₂ is independently (C₁-C₆)alkyl, (C₁-C₆)haloalkyl, (C₁-C₆)hydroxyalkyl, CN, or halogen, or
R₁ and R₂ together with the carbon atoms to which they are attached form a (C₃-C₇)cycloalkyl or a 4- to 6-membered heterocycloalkyl ring comprising 1 to 3 heteroatoms selected from O, N, and S, or
two R₂ together with the carbon atoms to which they are attached form (C₃-C₇)cycloalkyl or a 4- to 6-membered heterocycloalkyl ring comprising 1 to 3 heteroatoms selected from O, N, and S;
R₃ is (C₁-C₆)alkyl, (C₆-C₁₀)aryl, 5- or 6-membered heteroaryl comprising 1 to 3 heteroatoms selected from O, N, and S, (C₃-C₈)cycloalkyl, or 4- to 7-membered heterocycloalkyl comprising 1 to 3 heteroatoms selected from O, N, and S, wherein the alkyl is optionally substituted with one or more R₄; and the aryl, heteroaryl, cycloalkyl, and heterocycloalkyl are optionally substituted with one or more R₅, or
R₂ and R₃, when on adjacent atoms, together with the atoms to which they are attached form a 5- or 6-membered heterocycloalkyl ring;
each R₄ is independently selected from —C(O)OR₆, —C(O)NR₆R₆′, —NR₆C(O)R₆′, halogen, —OH, —NH₂, CN,
   (C₆-C₁₀)aryl, 5- or 6-membered heteroaryl comprising 1 to 4 heteroatoms selected from O, N, and S,
   (C₃-C₈)cycloalkyl, and 4- to 7-membered heterocycloalkyl ring comprising 1 to 3 heteroatoms selected from O, N, and S, wherein the aryl, heteroaryl, cycloalkyl, and heterocycloalkyl groups are optionally substituted with one or more R₇;
each R₅ is independently selected from (C₁-C₆)alkyl, (C₂-C₆)alkenyl, (C₂-C₆)alkynyl, (C₁-C₆)alkoxy,
   (C₁-C₆)haloalkyl, (C₁-C₆)haloalkoxy, (C₁-C₆)hydroxyalkyl, halogen, —OH, —NH₂, CN, (C₃-C₇)cycloalkyl, 5- to 7-membered heterocycloalkyl comprising 1 to 3 heteroatoms selected from O, N, and S, (C₆-C₁₀)aryl, and 5- or 6-membered heteroaryl comprising 1 to 3 heteroatoms selected from O, N, and S, or
two R₅, when on adjacent atoms, together with the atoms to which they are attached form a (C₆-C₁₀)aryl ring or a 5- or 6-membered heteroaryl ring comprising 1 to 3 heteroatoms selected from O, N, and S, optionally substituted with one or more R₁₀, or
two R₅ together with the atoms to which they are attached form a (C₃-C₇)cycloalkyl ring or a 4- to 7-membered heterocycloalkyl ring comprising 1 to 3 heteroatoms selected from O, N, and S optionally substituted with one or more R₁₀;
R₆ and R₆′ are each independently H, (C₁-C₆)alkyl, or (C₆-C₁₀)aryl;
each R₇ is independently selected from (C₁-C₆)alkyl, (C₂-C₆)alkenyl, (C₂-C₆)alkynyl, (C₁-C₆)alkoxy,
   (C₁-C₆)haloalkyl, (C₁-C₆)haloalkoxy, —C(O)R₈, —(CH₂)₀₋₃C(O)OR₈, —C(O)NR₈R₉, —NR₈C(O)R₉, —NR₈C(O)OR₉, —S(O)ₚNR₈R₉, —S(O)ₚR₁₂, (C₁-C₆)hydroxyalkyl, halogen, —OH, —O(CH₂)₁₋₃CN, —NH₂, CN, —O(CH₂)₀₋₃(C₆-C₁₀)aryl, adamantyl, —O(CH₂)₀₋₃-5- or 6-membered heteroaryl comprising 1 to 3 heteroatoms selected from O, N, and S, (C₆-C₁₀)aryl, monocyclic or bicyclic 5- to 10-membered heteroaryl comprising 1 to 3 heteroatoms selected from O, N, and S, (C₃-C₇)cycloalkyl, and 5- to 7-membered heterocycloalkyl comprising 1 to 3 heteroatoms selected from O, N, and S, wherein the alkyl is optionally substituted with one or more R₁₁, and the aryl, heteroaryl, and heterocycloalkyl are optionally substituted with one or more substituents each independently selected from halogen, (C₁-C₆)alkyl, (C₁-C₆)haloalkyl, and (C₁-C₆)alkoxy, or
two R₇ together with the carbon atom to which they are attached form a ═(O), or
two R₇, when on adjacent atoms, together with the atoms to which they are attached form a (C₆-C₁₀)aryl ring or a 5- or 6-membered heteroaryl ring comprising 1 to 3 heteroatoms selected from O, N, and S, optionally substituted with one or more R₁₀, or two R₇ together with the atoms to which they are attached form a (C₅-C₇) cycloalkyl ring or a 5- to 7-membered heterocycloalkyl ring comprising 1 to 3 heteroatoms selected from O, N, and S, optionally substituted with one or more R₁₀;
R₈ and R₉ are each independently H or (C₁-C₆)alkyl;
each R₁₀ is independently selected from (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —NH₂, and CN, or
two R₁₀ together with the carbon atom to which they are attached form a ═(O);
each R₁₁ is independently selected from CN, (C₁-C₆)alkoxy, (C₆-C₁₀)aryl, and 5- to 7-membered heterocycloalkyl comprising 1 to 3 heteroatoms selected from O, N, and S, wherein the aryl and heterocycloalkyl are optionally substituted with one or more substituents each independently selected from (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —NH₂, and CN;
R₁₂ is (C₁-C₆)alkyl, (C₁-C₆)haloalkyl, (C₆-C₁₀)aryl, or 5- to 7-membered heterocycloalkyl comprising 1 to 3 heteroatoms selected from O, N, and S;
Rₓ is H or D; and
n is 0, 1, 2, or 3;
   or pharmaceutically acceptable salts, hydrates, solvates, prodrugs, stereoisomers, and tautomers thereof.

In one aspect of the disclosure, the hydrogens in the compound of Formula (I) are present in their normal isotopic abundances. In a preferred aspect of the disclosure, the hydrogens are isotopically enriched in deuterium (D), and in a particularly preferred aspect of the invention the hydrogen at position Rₓ is enriched in D, as discussed in more detail concerning isotopes and isotopic enrichment below.

In another aspect, the present disclosure relates to a compound selected from:

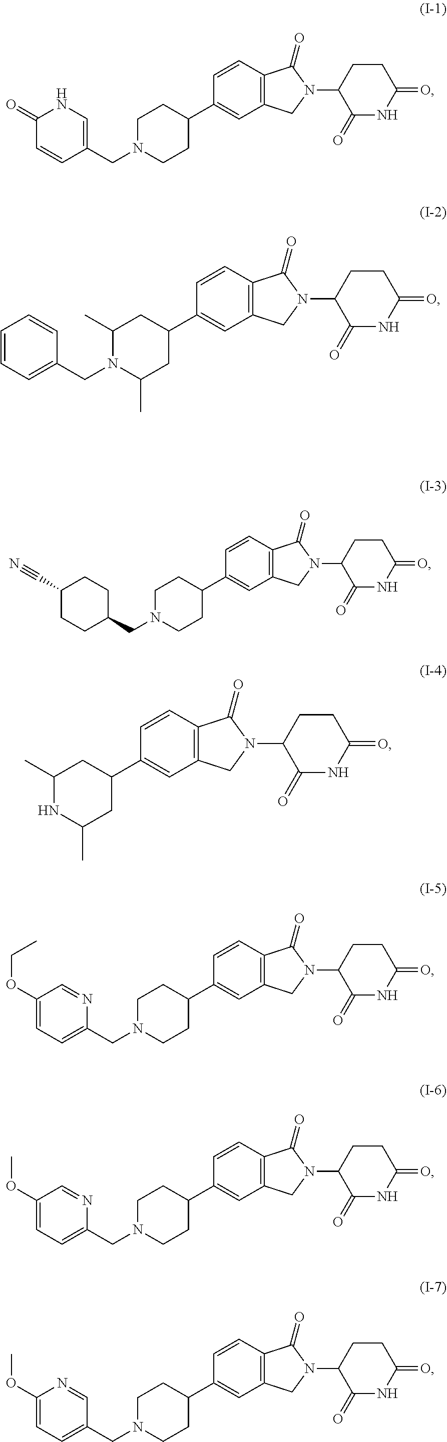

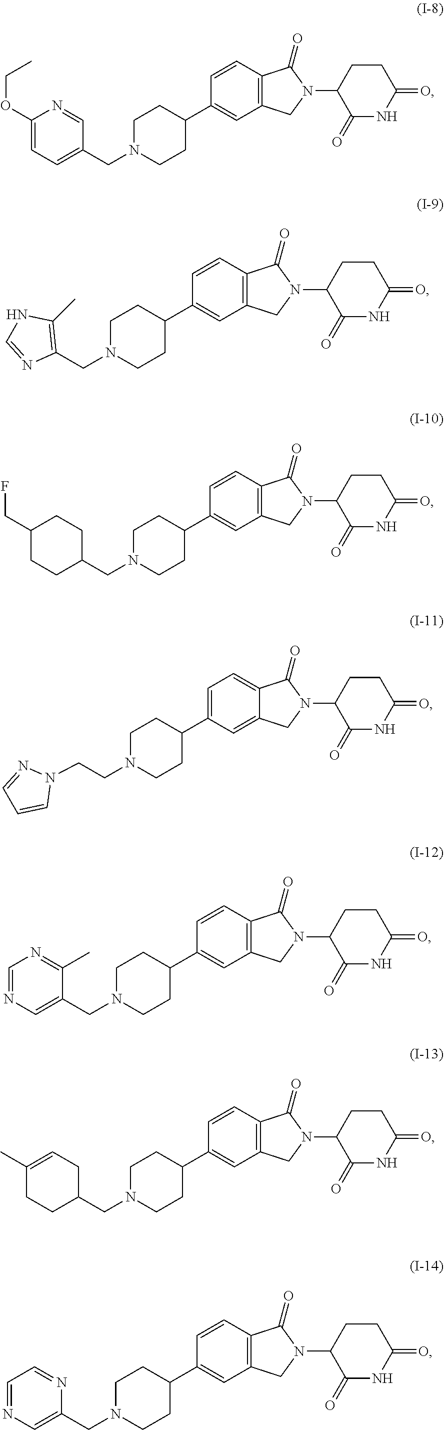

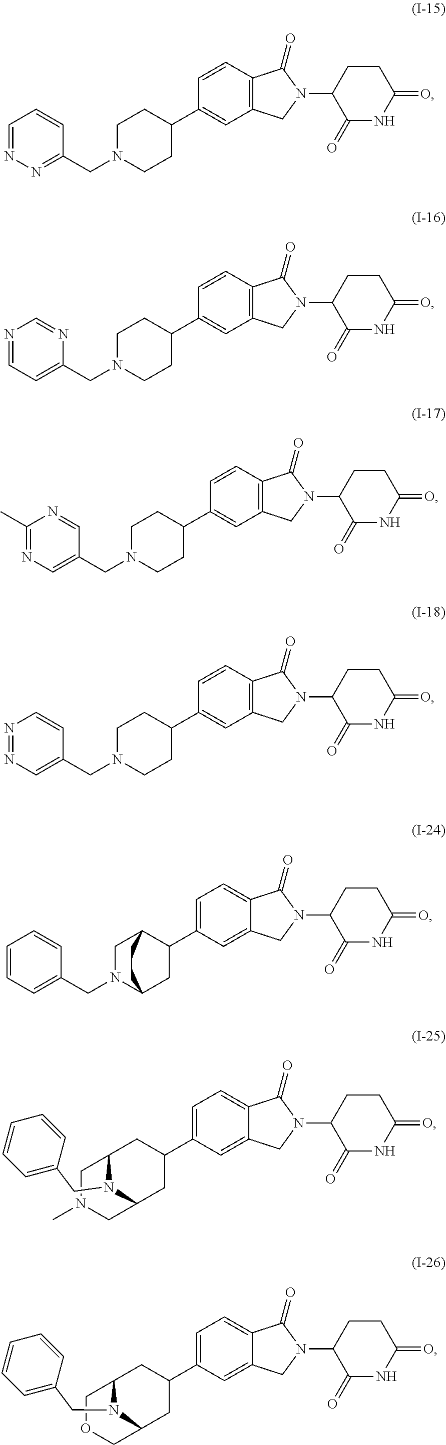

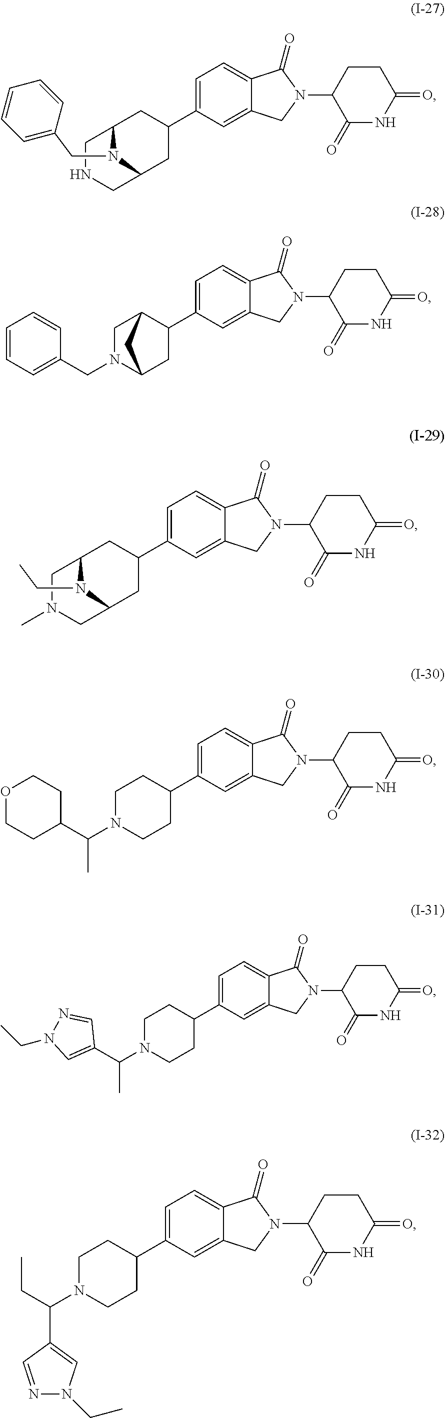 [SMILES: O=C1CCC(N2Cc3cc(C4C[C@H]5CNC[C@@H](C4)N5Cc4ccccc4)ccc3C2=O)C(=O)N1 ; O=C1CCC(N2Cc3cc(C4C[C@H]5C[C@@H]4CN5Cc4ccccc4)ccc3C2=O)C(=O)N1 ; CCN1[C@@H]2CC(c3ccc4c(c3)CN(C3CCC(=O)NC3=O)C4=O)C[C@H]1CN(C)C2 ; CC(C1CCOCC1)N1CCC(c2ccc3c(c2)CN(C2CCC(=O)NC2=O)C3=O)CC1 ; CCn1cc(C(C)N2CCC(c3ccc4c(c3)CN(C3CCC(=O)NC3=O)C4=O)CC2)cn1 ; CCC(c1cnn(CC)c1)N1CCC(c2ccc3c(c2)CN(C2CCC(=O)NC2=O)C3=O)CC1]

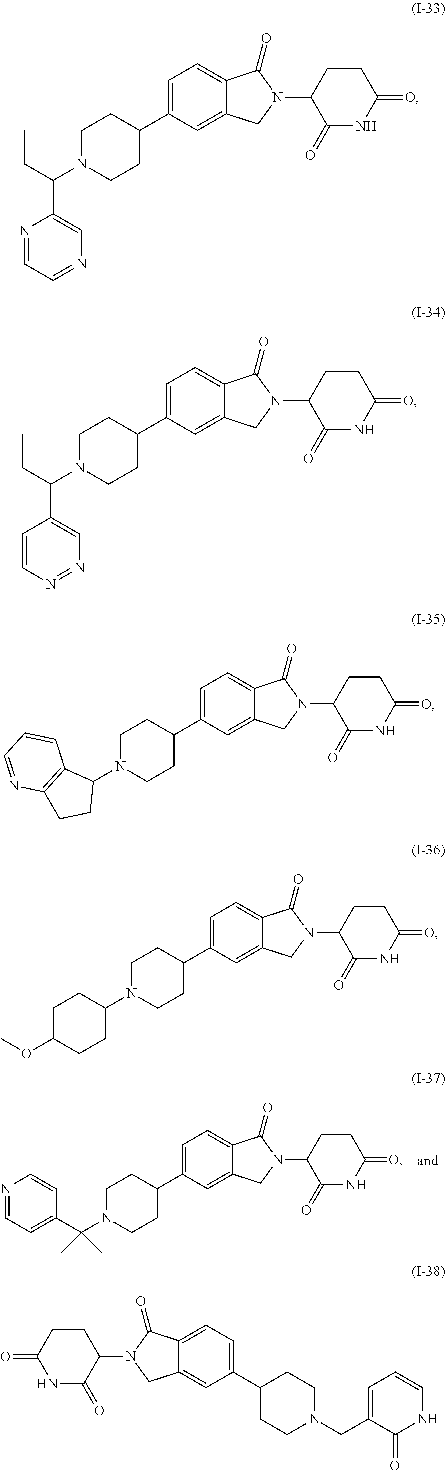 [SMILES: CCC(c1cnccn1)N1CCC(c2ccc3c(c2)CN(C2CCC(=O)NC2=O)C3=O)CC1 ; CCC(c1ccnnc1)N1CCC(c2ccc3c(c2)CN(C2CCC(=O)NC2=O)C3=O)CC1 ; O=C1CCC(N2Cc3cc(C4CCN(C5CCc6ncccc65)CC4)ccc3C2=O)C(=O)N1 ; COC1CCC(N2CCC(c3ccc4c(c3)CN(C3CCC(=O)NC3=O)C4=O)CC2)CC1 ; CC(C)(c1ccncc1)N1CCC(c2ccc3c(c2)CN(C2CCC(=O)NC2=O)C3=O)CC1 ; O=C1CCC(N2Cc3cc(C4CCN(Cc5ccc[nH]c5=O)CC4)ccc3C2=O)C(=O)N1]

or pharmaceutically acceptable salts, hydrates, solvates, prodrugs, stereoisomers, and tautomers thereof.

Another aspect of the present disclosure relates to a pharmaceutical composition comprising a therapeutically effective amount of a compound of Formula (I), or a pharmaceutically acceptable salt, hydrate, solvate, prodrug, stereoisomer, or tautomer thereof, and a pharmaceutically acceptable carrier or excipient. The pharmaceutical composition is useful in the treatment of IKZF2-dependent diseases or disorders. The pharmaceutical composition may further comprise at least one additional pharmaceutical agent.

In another aspect, the present disclosure relates to a pharmaceutical composition comprising a therapeutically effective amount of a compound of Formula (I), or a pharmaceutically acceptable salt, hydrate, solvate, prodrug, stereoisomer, or tautomer thereof, and a pharmaceutically acceptable carrier or excipient for use in the treatment of an IKZF2-dependent disease or disorder by reducing IKZF2 protein levels wherein reduction of IKZF2 protein levels treats the IKZF2-dependent disease or disorder. The pharmaceutical composition is useful in the treatment of IKZF2-dependent diseases or disorders. The pharmaceutical composition may further comprise at least one additional pharmaceutical agent.

Another aspect of the present disclosure relates to a pharmaceutical composition comprising a therapeutically effective amount of a compound of Formula (I), or a pharmaceutically acceptable salt, hydrate, solvate, prodrug, stereoisomer, or tautomer thereof, and a pharmaceutically acceptable carrier or excipient. The pharmaceutical composition is useful in the treatment of diseases or disorders affected by the reduction of IKZF2 protein levels. The pharmaceutical composition may further comprise at least one additional pharmaceutical agent.

In another aspect, the present disclosure relates to a pharmaceutical composition comprising a therapeutically effective amount of a compound of Formula (I), or a pharmaceutically acceptable salt, hydrate, solvate, prodrug, stereoisomer, or tautomer thereof, and a pharmaceutically acceptable carrier or excipient for use in the treatment of a disease or disorder affected by the reduction of IKZF2 protein levels wherein reduction of IKZF2 protein levels treats the disease or disorder. The pharmaceutical composition may further comprise at least one additional pharmaceutical agent.

Another aspect of the present disclosure relates to a pharmaceutical composition comprising a therapeutically effective amount of a compound selected from compound (I-1), (I-2), (I-3), (I-4), (I-5), (I-6), (I-7), (I-8), (I-9), (I-10), (I-11), (I-12), (I-13), (I-14), (I-15), (I-16), (I-17), (I-18), (I-24), (I-25), (I-26), (I-27), (I-28), (I-29), (I-30), (I-31), (I-32), (I-33), (I-34), (I-34), (I-36), (I-37), and (I-38), or a pharmaceutically acceptable salt, hydrate, solvate, prodrug, stereoisomer, or tautomer thereof, and a pharmaceutically acceptable carrier or excipient. The pharmaceutical composition is useful in the treatment of IKZF2-dependent diseases or disorders.

The pharmaceutical composition may further comprise at least one additional pharmaceutical agent.

In another aspect, the present disclosure relates to a pharmaceutical composition comprising a therapeutically effective amount of a compound selected from compound (I-1), (I-2), (I-3), (I-4), (I-5), (I-6), (I-7), (I-8), (I-9), (I-10), (I-11), (I-12), (I-13), (I-14), (I-15), (I-16), (I-17), (I-18), (I-24), (I-25), (I-26), (I-27), (I-28), (I-29), (I-30), (I-31), (I-32), (I-33), (I-34), (I-34), (I-36), (I-37), and (I-38), or a pharmaceutically acceptable salt, hydrate, solvate, prodrug, stereoisomer, or tautomer thereof, and a pharmaceutically acceptable carrier or excipient for use in the treatment of an IKZF2-dependent disease or disorder by reducing IKZF2 protein levels wherein reduction of IKZF2 protein levels treats the IKZF2-dependent disease or disorder. The pharmaceutical composition is useful in the treatment of IKZF2-dependent diseases or disorders. The pharmaceutical composition may further comprise at least one additional pharmaceutical agent.

Another aspect of the present disclosure relates to a pharmaceutical composition comprising a therapeutically effective amount of a compound selected from compound (I-1), (I-2), (I-3), (I-4), (I-5), (I-6), (I-7), (I-8), (I-9), (I-10), (I-11), (I-12), (I-13), (I-14), (I-15), (I-16), (I-17), (I-18), (I-24), (I-25), (I-26), (I-27), (I-28), (I-29), (I-30), (I-31), (I-32), (I-33), (I-34), (I-34), (I-36), (I-37), and (I-38), or a pharmaceutically acceptable salt, hydrate, solvate, prodrug, stereoisomer, or tautomer thereof, and a pharmaceutically acceptable carrier or excipient. The pharmaceutical composition is useful in the treatment of diseases or disorders affected by the reduction of IKZF2 protein levels. The pharmaceutical composition may further comprise at least one additional pharmaceutical agent.

In another aspect, the present disclosure relates to a pharmaceutical composition comprising a therapeutically effective amount of a compound selected from compound (I-1), (I-2), (I-3), (I-4), (I-5), (I-6), (I-7), (I-8), (I-9), (I-10), (I-11), (I-12), (I-13), (I-14), (I-15), (I-16), (I-17), (I-18), (I-24), (I-25), (I-26), (I-27), (I-28), (I-29), (I-30), (I-31), (I-32), (I-33), (I-34), (I-34), (I-36), (I-37), and (I-38), or a pharmaceutically acceptable salt, hydrate, solvate, prodrug, stereoisomer, or tautomer thereof, and a pharmaceutically acceptable carrier or excipient for use in the treatment of a disease or disorder affected by the reduction of IKZF2 protein levels wherein reduction of IKZF2 protein levels treats the disease or disorder. The pharmaceutical composition may further comprise at least one additional pharmaceutical agent.

Another aspect of the present disclosure relates to a method of degrading IKZF2 comprising administering to the patient in need thereof a compound of Formula (I), or a pharmaceutically acceptable salt, hydrate, solvate, prodrug, stereoisomer, or tautomer thereof.

In another aspect, the present disclosure relates to a method of treating a disease or disorder that is affected by the modulation of IKZF2 protein levels comprising administering to the patient in need thereof a compound of Formula (I), or a pharmaceutically acceptable salt, hydrate, solvate, prodrug, stereoisomer, or tautomer thereof.

Another aspect of the present disclosure relates to a method of modulating IKZF2 protein levels comprising administering to the patient in need thereof a compound of Formula (I), or a pharmaceutically acceptable salt, hydrate, solvate, prodrug, stereoisomer, or tautomer thereof.

In another aspect, the present disclosure relates to a method of reducing the proliferation of a cell the method comprising, contacting the cell with a compound of Formula (I), or a pharmaceutically acceptable salt, hydrate, solvate, prodrug, stereoisomer, or tautomer thereof, and reducing IKZF2 protein levels.

Another aspect of the present disclosure relates to a method of treating cancer comprising administering to the patient in need thereof a compound of Formula (I), or a pharmaceutically acceptable salt, hydrate, solvate, prodrug, stereoisomer, or tautomer thereof. In one embodiment, the cancer is selected from non-small cell lung cancer (NSCLC), melanoma, triple-negative breast cancer (TNBC), nasopharyngeal cancer (NPC), microsatellite stable colorectal cancer (mssCRC), thymoma, carcinoid, acute myelogenous leukemia, and gastrointestinal stromal tumor (GIST). In another embodiment, the cancer is a cancer for which the immune response is deficient or an immunogenic cancer.

In another aspect, the present disclosure relates to a method for reducing IKZF2 protein levels in a subject comprising the step of administering to a subject in need thereof a therapeutically effective amount of a compound of Formula (I), or a pharmaceutically acceptable salt.

Another aspect of the present disclosure relates to a compound of Formula (I), or a pharmaceutically acceptable salt, hydrate, solvate, prodrug, stereoisomer, or tautomer thereof, for use in the treatment of a disease or disorder that is affected by the reduction of IKZF2 protein levels.

In another aspect, the present disclosure relates to the use of a compound of Formula (I), or a pharmaceutically acceptable salt, hydrate, solvate, prodrug, stereoisomer, or tautomer thereof, in the manufacture of a medicament for treating a disease or disorder that is affected by the reduction of IKZF2 protein levels.

Another aspect of the present disclosure relates to a compound of Formula (I), or a pharmaceutically acceptable salt, hydrate, solvate, prodrug, stereoisomer, or tautomer thereof, for use in the manufacture of a medicament for treating a disease or disorder associated with the reduction of IKZF2 protein levels. In one embodiment, the disease or disorder is selected from non-small cell lung cancer (NSCLC), melanoma, triple-negative breast cancer (TNBC), nasopharyngeal cancer (NPC), microsatellite stable colorectal cancer (mssCRC), thymoma, carcinoid, acute myelogenous leukemia, and gastrointestinal stromal tumor (GIST).

In another aspect, the present disclosure relates to the use of a compound of Formula (I), or a pharmaceutically acceptable salt, hydrate, solvate, prodrug, stereoisomer, or tautomer thereof, in the treatment of a disease or disorder associated with the reduction of IKZF2 protein levels. In one embodiment, the disease or disorder is selected from non-small cell lung cancer (NSCLC), melanoma, triple-negative breast cancer (TNBC), nasopharyngeal cancer (NPC), microsatellite stable colorectal cancer (mssCRC), thymoma, carcinoid, acute myelogenous leukemia, and gastrointestinal stromal tumor (GIST).

Another aspect of the present disclosure relates to a method of degrading IKZF2 comprising administering to the patient in need thereof a compound selected from compound (I-1), (I-2), (I-3), (I-4), (I-5), (I-6), (I-7), (I-8), (I-9), (I-10), (I-11), (I-12), (I-13), (I-14), (I-15), (I-16), (I-17), (I-18), (I-24), (I-25), (I-26), (I-27), (I-28), (I-29), (I-30), (I-31), (I-32), (I-33), (I-34), (I-34), (I-36), (I-37), and (I-38), or a pharmaceutically acceptable salt, hydrate, solvate, prodrug, stereoisomer, or tautomer thereof.

In another aspect, the present disclosure relates to a method of treating a disease or disorder that is affected by the modulation of IKZF2 protein levels comprising administering to the patient in need thereof a compound selected from compound (I-1), (I-2), (I-3), (I-4), (I-5), (I-6), (I-7), (I-8), (I-9), (I-10), (I-11), (I-12), (I-13), (I-14), (I-15), (I-16), (I-17), (I-18), (I-24), (I-25), (I-26), (I-27), (I-28), (I-29), (I-30), (I-31), (I-32), (I-33), (I-34), (I-34), (I-36), (I-37), and (I-38), or a pharmaceutically acceptable salt, hydrate, solvate, prodrug, stereoisomer, or tautomer thereof.

Another aspect of the present disclosure relates to a method of modulating IKZF2 protein levels comprising administering to the patient in need thereof a compound selected from compound (I-1), (I-2), (I-3), (I-4), (I-5), (I-6), (I-7), (I-8), (I-9), (I-10), (I-11), (I-12), (I-13), (I-14), (I-15), (I-16), (I-17), (I-18), (I-24), (I-25), (I-26), (I-27), (I-28), (I-29), (I-30), (I-31), (I-32), (I-33), (I-34), (I-34), (I-36), (I-37), and (I-38), or a pharmaceutically acceptable salt, hydrate, solvate, prodrug, stereoisomer, or tautomer thereof.

In another aspect, the present disclosure relates to a method of reducing the proliferation of a cell the method comprising, contacting the cell with a compound selected from compound (I-1), (I-2), (I-3), (I-4), (I-5), (I-6), (I-7), (I-8), (I-9), (I-10), (I-11), (I-12), (I-13), (I-14), (I-15), (I-16), (I-17), (I-18), (I-24), (I-25), (I-26), (I-27), (I-28), (I-29), (I-30), (I-31), (I-32), (I-33), (I-34), (I-34), (I-36), (I-37), and (I-38), or a pharmaceutically acceptable salt, hydrate, solvate, prodrug, stereoisomer, or tautomer thereof, and reducing IKZF2 protein levels.

Another aspect of the present disclosure relates to a method of treating cancer comprising administering to the patient in need thereof a compound selected from compound (I-1), (I-2), (I-3), (I-4), (I-5), (I-6), (I-7), (I-8), (I-9), (I-10), (I-11), (I-12), (I-13), (I-14), (I-15), (I-16), (I-17), (I-18), (I-24), (I-25), (I-26), (I-27), (I-28), (I-29), (I-30), (I-31), (I-32), (I-33), (I-34), (I-34), (I-36), (I-37), and (I-38), or a pharmaceutically acceptable salt, hydrate, solvate, prodrug, stereoisomer, or tautomer thereof. In one embodiment, the cancer is selected from non-small cell lung cancer (NSCLC), melanoma, triple-negative breast cancer (TNBC), nasopharyngeal cancer (NPC), microsatellite stable colorectal cancer (mssCRC), thymoma, carcinoid, acute myelogenous leukemia, and gastrointestinal stromal tumor (GIST). In another embodiment, the cancer is a cancer for which the immune response is deficient or an immunogenic cancer.

In another aspect, the present disclosure relates to a method for reducing IKZF2 protein levels in a subject comprising the step of administering to a subject in need thereof a therapeutically effective amount of a compound selected from compound (I-1), (I-2), (I-3), (I-4), (I-5), (I-6), (I-7), (I-8), (I-9), (I-10), (I-11), (I-12), (I-13), (I-14), (I-15), (I-16), (I-17), (I-18), (I-24), (I-25), (I-26), (I-27), (I-28), (I-29), (I-30), (I-31), (I-32), (I-33), (I-34), (I-34), (I-36), (I-37), and (I-38), or a pharmaceutically acceptable salt.

Another aspect of the present disclosure relates to a compound selected from compound (I-1), (I-2), (I-3), (I-4), (I-5), (I-6), (I-7), (I-8), (I-9), (I-10), (I-11), (I-12), (I-13), (I-14), (I-15), (I-16), (I-17), (I-18), (I-24), (I-25), (I-26), (I-27), (I-28), (I-29), (I-30), (I-31), (I-32), (I-33), (I-34), (I-34), (I-36), (I-37), and (I-38), or a pharmaceutically acceptable salt, hydrate, solvate, prodrug, stereoisomer, or tautomer thereof, for use in the treatment of a disease or disorder that is affected by the reduction of IKZF2 protein levels.

In another aspect, the present disclosure relates to the use of a compound selected from compound (I-1), (I-2), (I-3), (I-4), (I-5), (I-6), (I-7), (I-8), (I-9), (I-10), (I-11), (I-12), (I-13), (I-14), (I-15), (I-16), (I-17), (I-18), (I-24), (I-25), (I-26), (I-27), (I-28), (I-29), (I-30), (I-31), (I-32), (I-33), (I-34), (I-34), (I-36), (I-37), and (I-38), or a pharmaceutically acceptable salt, hydrate, solvate, prodrug, stereoisomer, or tautomer thereof, in the manufacture of a medicament for treating a disease or disorder that is affected by the reduction of IKZF2 protein levels.

Another aspect of the present disclosure relates to a compound selected from compound (I-1), (I-2), (I-3), (I-4), (I-5), (I-6), (I-7), (I-8), (I-9), (I-10), (I-11), (I-12), (I-13), (I-14), (I-15), (I-16), (I-17), (I-18), (I-24), (I-25), (I-26), (I-27), (I-28), (I-29), (I-30), (I-31), (I-32), (I-33), (I-34), (I-34), (I-36), (I-37), and (I-38), or a pharmaceutically acceptable salt, hydrate, solvate, prodrug, stereoisomer, or tautomer thereof, for use in the manufacture of a medicament for treating a disease or disorder associated with the reduction of IKZF2 protein levels. In one embodiment, the disease or disorder is selected from non-small cell lung cancer (NSCLC), melanoma, triple-negative breast cancer (TNBC), nasopharyngeal cancer (NPC), microsatellite stable colorectal cancer (mssCRC), thymoma, carcinoid, acute myelogenous leukemia, and gastrointestinal stromal tumor (GIST).

In another aspect, the present disclosure relates to the use of a compound selected from compound (I-1), (I-2), (I-3), (I-4), (I-5), (I-6), (I-7), (I-8), (I-9), (I-10), (I-11), (I-12), (I-13), (I-14), (I-15), (I-16), (I-17), (I-18), (I-24), (I-25), (I-26), (I-27), (I-28), (I-29), (I-30), (I-31), (I-32), (I-33), (I-34), (I-34), (I-36), (I-37), and (I-38), or a pharmaceutically acceptable salt, hydrate, solvate, prodrug, stereoisomer, or tautomer thereof, in the treatment of a disease or disorder associated with the reduction of IKZF2 protein levels. In one embodiment, the disease or disorder is selected from non-small cell lung cancer (NSCLC), melanoma, triple-negative breast cancer (TNBC), nasopharyngeal cancer (NPC), microsatellite stable colorectal cancer (mssCRC), thymoma, carcinoid, acute myelogenous leukemia, and gastrointestinal stromal tumor (GIST).

In another aspect of the disclosure, the compounds according to the disclosure are formulated into pharmaceutical compositions comprising an effective amount, preferably a pharmaceutically effective amount, of a compound according to the disclosure or salt, hydrate, solvate, prodrug, stereoisomer, or tautomer thereof, and a pharmaceutically acceptable excipient or carrier.

In some embodiments of the methods disclosed herein, the administration of the compound of Formula (I), or a pharmaceutically acceptable salt, hydrate, solvate, prodrug, stereoisomer, or tautomer thereof, is performed orally, parentally, subcutaneously, by injection, or by infusion.

In some embodiments of the methods disclosed herein, the administration of the compound selected from compound (I-1), (I-2), (I-3), (I-4), (I-5), (I-6), (I-7), (I-8), (I-9), (I-10), (I-11), (I-12), (I-13), (I-14), (I-15), (I-16), (I-17), (I-18), (I-24), (I-25), (I-26), (I-27), (I-28), (I-29), (I-30), (I-31), (I-32), (I-33), (I-34), (I-34), (I-36), (I-37), and (I-38), or a pharmaceutically acceptable salt, hydrate, solvate, prodrug, stereoisomer, or tautomer thereof, is performed orally, parentally, subcutaneously, by injection, or by infusion.

The present disclosure provides degraders of IKZF2 that are therapeutic agents in the treatment of diseases such as cancer and metastasis, in the treatment of diseases affected by the modulation of IKZF2 protein levels, and in the treatment IKZF2-dependent diseases or disorders.

In one embodiment, the disease or disorder that can be treated by the compounds of the present disclosure is selected from non-small cell lung cancer (NSCLC), melanoma, triple-negative breast cancer (TNBC), nasopharyngeal cancer (NPC), microsatellite stable colorectal cancer (mssCRC), thymoma, carcinoid, gastrointestinal stromal tumor (GIST), prostate cancer, breast carcinoma, lymphomas, leukaemia, myeloma, bladder carcinoma, colon cancer, cutaneous melanoma, hepatocellular carcinoma, endometrial cancer, ovarian cancer, cervical cancer, lung cancer, renal cancer, glioblastoma multiform, glioma, thyroid cancer, parathyroid tumor, nasopharyngeal cancer, tongue cancer, pancreatic cancer, esophageal cancer, cholangiocarcinoma, gastric cancer, soft tissue sarcomas, rhabdomyosarcoma (RMS), synovial sarcoma, osteosarcoma, rhabdoid cancers, and Ewing's sarcoma. In another embodiment, the IKZF2-dependent disease or disorder is a cancer for which the immune response is deficient or an immunogenic cancer.

The present disclosure provides agents with novel mechanisms of action toward IKZF2 proteins in the treatment of various types of diseases including cancer and metastasis, in the treatment of diseases affected by the modulation of IKZF2 protein levels, and in the treatment IKZF2-dependent diseases or disorders. Ultimately the present disclosure provides the medical community with a novel pharmacological strategy for the treatment of diseases and disorders associated with IKZF2 proteins.

The present disclosure provides agents with novel mechanisms of action toward IKZF2 proteins in the treatment of various types of diseases including cancer and metastasis, in the treatment of diseases affected by the modulation of IKZF2 protein levels, and in the treatment IKZF2-dependent diseases or disorders. Ultimately, the present disclosure provides the medical community with a novel pharmacological strategy for the treatment of diseases and disorders associated with IKZF2 proteins.

### DETAILED DESCRIPTION OF THE DISCLOSURE

The present disclosure relates to compounds and compositions that are capable of modulating IKZF2 protein levels. The disclosure features methods of treating, preventing, or ameliorating a disease or disorder in which IKZF2 plays a role by administering to a patient in need thereof a therapeutically effective amount of a compound of Formula (I), or a pharmaceutically acceptable salt, hydrate, solvate, prodrug, stereoisomer, or tautomer thereof. The methods of the present disclosure can be used in the treatment of a variety of IKZF2-dependent diseases and disorders by modulating IKZF2 protein levels. Modulation of IKZF2 protein levels through degradation provides a novel approach to the treatment, prevention, or amelioration of diseases including, but not limited to, cancer and metathesis, and other IKZF2-dependent diseases or disorders.

In one aspect, the compounds of the disclosure have use as therapeutic agents, particularly for cancers and related diseases. In one aspect, the compounds of the disclosure have IKZF2 degradation activity, preferably having such activity at or below the 50 μM level, and more preferably having such activity at or below the 10 μM level. In another aspect, the compounds of the disclosure have degrader activity for IKZF2 that is selective over one or more of IKZF1, IKZF3, IKZF4, and/or IKZF5. In another aspect, the compounds of the disclosure have degrader activity for both IKZF2 and IKZF4. The compounds of the disclosure have usefulness in treating cancer and other diseases for which such degradation activity would be beneficial for the patient. For example, while not intending to be bound by any theory, the inventors believe that reducing levels of IKZF2 in Tregs in a tumor may allow the patient immune system to more effectively attack the disease. In summary, the present disclosure provides novel IKZF2 degraders useful for the treatment of cancer and other diseases.

In a first aspect of the disclosure, the compounds of Formula (I) are described:

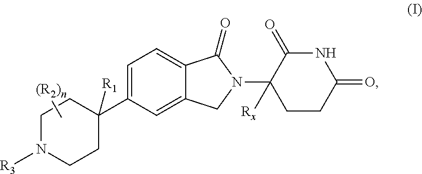

or pharmaceutically acceptable salts, hydrates, solvates, prodrugs, stereoisomers, and tautomers thereof, wherein R₁, R₂, R₃, Rₓ, and n are as described herein above.

The details of the disclosure are set forth in the accompanying description below. Although methods and materials similar or equivalent to those described herein can be used in the practice or testing of the present disclosure, illustrative methods and materials are now described. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs. All patents and publications cited in this specification are incorporated herein by reference in their entireties.

### Definition of Terms and Conventions Used

Terms not specifically defined herein should be given the meanings that would be given to them by one of skill in the art in light of the disclosure and the context. As used in the specification and appended claims, however, unless specified to the contrary, the following terms have the meaning indicated and the following conventions are adhered to.

### A. Chemical Nomenclature, Terms, and Conventions

In the groups, radicals, or moieties defined below, the number of carbon atoms is often specified preceding the group, for example, (C₁-C₁₀)alkyl means an alkyl group or radical having 1 to 10 carbon atoms. In general, for groups comprising two or more subgroups, the last named group is the radical attachment point, for example, “alkylaryl” means a monovalent radical of the formula alkyl-aryl-, while “arylalkyl” means a monovalent radical of the formula aryl-alkyl-. Furthermore, the use of a term designating a monovalent radical where a divalent radical is appropriate shall be construed to designate the respective divalent radical and vice versa. Unless otherwise specified, conventional definitions of terms control and conventional stable atom valences are presumed and achieved in all formulas and groups. The articles “a” and “an” refer to one or more than one (e.g., to at least one) of the grammatical object of the article. By way of example, “an element” means one element or more than one element.

The term “and/or” means either “and” or “or” unless indicated otherwise.

The term “optionally substituted” means that a given chemical moiety (e.g., an alkyl group) can (but is not required to) be bonded other substituents (e.g., heteroatoms). For instance, an alkyl group that is optionally substituted can be a fully saturated alkyl chain (e.g., a pure hydrocarbon).

Alternatively, the same optionally substituted alkyl group can have substituents different from hydrogen. For instance, it can, at any point along the chain be bounded to a halogen atom, a hydroxyl group, or any other substituent described herein. Thus, the term “optionally substituted” means that a given chemical moiety has the potential to contain other functional groups, but does not necessarily have any further functional groups. Suitable substituents used in the optional substitution of the described groups include, without limitation, halogen, oxo, —OH, —CN, —COOH, —CH₂CN, —O—(C₁-C₆)alkyl, (C₁-C₆)alkyl, (C₁-C₆)alkoxy, (C₁-C₆)haloalkyl, (C₁-C₆)haloalkoxy, —O—(C₂-C₆)alkenyl, —O—(C₂-C₆)alkynyl, (C₂-C₆)alkenyl, (C₂-C₆)alkynyl, —OH, —OP(O)(OH)₂, —OC(O)(C₁-C₆)alkyl, —C(O)(C₁-C₆)alkyl, —OC(O)O(C₁-C₆)alkyl, —NH₂, —NH((C₁-C₆)alkyl), —N((C₁-C₆)alkyl)₂, —NHC(O)(C₁-C₆)alkyl, —C(O)NH(C₁-C₆)alkyl, —S(O)₂(C₁-C₆)alkyl, —S(O)NH(C₁-C₆)alkyl, and S(O)N((C₁-C₆)alkyl)₂. The substituents can themselves be optionally substituted. “Optionally substituted” as used herein also refers to substituted or unsubstituted whose meaning is described below.

The term “substituted” means that the specified group or moiety bears one or more suitable substituents wherein the substituents may connect to the specified group or moiety at one or more positions. For example, an aryl substituted with a cycloalkyl may indicate that the cycloalkyl connects to one atom of the aryl with a bond or by fusing with the aryl and sharing two or more common atoms.

The term “unsubstituted” means that the specified group bears no substituents.

Unless otherwise specifically defined, “aryl” means a cyclic, aromatic hydrocarbon group having 1 to 3 aromatic rings, including monocyclic or bicyclic groups such as phenyl, biphenyl, or naphthyl. When containing two aromatic rings (bicyclic, etc.), the aromatic rings of the aryl group are optionally joined at a single point (e.g., biphenyl), or fused (e.g., naphthyl). The aryl group is optionally substituted by one or more substituents, e.g., 1 to 5 substituents, at any point of attachment. Exemplary substituents include, but are not limited to, —H, -halogen, —CN, —O—(C₁-C₆)alkyl, (C₁-C₆)alkyl, —O—(C₂-C₆)alkenyl, —O—(C₂-C₆)alkynyl, (C₂-C₆)alkenyl, (C₂-C₆)alkynyl, —OH, —OP(O)(OH)₂, —OC(O)(C₁-C₆)alkyl, —C(O)(C₁-C₆)alkyl, —OC(O)O(C₁-C₆)alkyl, NH₂, NH((C₁-C₆)alkyl), N((C₁-C₆)alkyl)₂, —S(O)₂—(C₁-C₆)alkyl, —S(O)NH(C₁-C₆)alkyl, and S(O)N((C₁-C₆)alkyl)₂. The substituents are themselves optionally substituted. Furthermore, when containing two fused rings, the aryl groups optionally have an unsaturated or partially saturated ring fused with a fully saturated ring. Exemplary ring systems of these aryl groups include, but are not limited to, phenyl, biphenyl, naphthyl, anthracenyl, phenalenyl, phenanthrenyl, indanyl, indenyl, tetrahydronaphthalenyl, tetrahydrobenzoannulenyl, and the like.

Unless otherwise specifically defined, “heteroaryl” means a monovalent monocyclic aromatic radical of 5 to 24 ring atoms or a polycyclic aromatic radical, containing one or more ring heteroatoms selected from N, O, or S, the remaining ring atoms being C. Heteroaryl as herein defined also means a bicyclic heteroaromatic group wherein the heteroatom is selected from N, O, or S. The aromatic radical is optionally substituted independently with one or more substituents described herein. Examples include, but are not limited to, furyl, thienyl, pyrrolyl, pyridyl, pyrazolyl, pyrimidinyl, imidazolyl, isoxazolyl, oxazolyl, oxadiazolyl, pyrazinyl, indolyl, thiophen-2-yl, quinolyl, benzopyranyl, isothiazolyl, thiazolyl, thiadiazole, indazole, benzimidazolyl, thieno[3,2-b]thiophene, triazolyl, triazinyl, imidazo[1,2-b]pyrazolyl, furo[2,3-c]pyridinyl, imidazo[1,2-a]pyridinyl, indazolyl, pyrrolo[2,3-c]pyridinyl, pyrrolo[3,2-c]pyridinyl, pyrazolo[3,4-c]pyridinyl, thieno[3,2-c]pyridinyl, thieno[2,3-c]pyridinyl, thieno[2,3-b]pyridinyl, benzothiazolyl, indolyl, indolinyl, indolinonyl, dihydrobenzothiophenyl, dihydrobenzofuranyl, benzofuran, chromanyl, thiochromanyl, tetrahydroquinolinyl, dihydrobenzothiazine, dihydrobenzoxanyl, quinolinyl, isoquinolinyl, 1,6-naphthyridinyl, benzo[de]isoquinolinyl, pyrido[4,3-b][1,6]naphthyridinyl, thieno[2,3-b]pyrazinyl, quinazolinyl, tetrazolo[1,5-a]pyridinyl, [1,2,4]triazolo[4,3-a]pyridinyl, isoindolyl, pyrrolo[2,3-b]pyridinyl, pyrrolo[3,4-b]pyridinyl, pyrrolo[3,2-b]pyridinyl, imidazo[5,4-b]pyridinyl, pyrrolo[1,2-a]pyrimidinyl, tetrahydropyrrolo[1,2-a]pyrimidinyl, 3,4-dihydro-2H-1Δ²-pyrrolo[2,1-b]pyrimidine, dibenzo[b,d]thiophene, pyridin-2-one, furo[3,2-c]pyridinyl, furo[2,3-c]pyridinyl, 1H-pyrido[3,4-b][1,4]thiazinyl, benzooxazolyl, benzoisoxazolyl, furo[2,3-b]pyridinyl, benzothiophenyl, 1,5-naphthyridinyl, furo[3,2-b]pyridine, [1,2,4]triazolo[1,5-a]pyridinyl, benzo[1,2,3]triazolyl, imidazo[1,2-a]pyrimidinyl, [1,2,4]triazolo[4,3-b]pyridazinyl, benzo[c][1,2,5]thiadiazolyl, benzo[c][1,2,5]oxadiazole, 1,3-dihydro-2H-benzo[d]imidazol-2-one, 3,4-dihydro-2H-pyrazolo[1,5-b][1,2]oxazinyl, 4,5,6,7-tetrahydropyrazolo[1,5-a]pyridinyl, thiazolo[5,4 d]thiazolyl, imidazo[2,1-b][1,3,4]thiadiazolyl, thieno[2,3-b]pyrrolyl, 3H-indolyl, and derivatives thereof.

Furthermore, when containing two fused rings the aryl groups herein defined may have an unsaturated or partially saturated ring fused with a fully saturated ring. Exemplary ring systems of these heteroaryl groups include indolinyl, indolinonyl, dihydrobenzothiophenyl, dihydrobenzofuran, chromanyl, thiochromanyl, tetrahydroquinolinyl, dihydrobenzothiazine,3,4-dihydro-1H-isoquinolinyl, 2,3-dihydrobenzofuran, indolinyl, indolyl, and dihydrobenzoxanyl.

Halogen or “halo” mean fluorine, chlorine, bromine, or iodine.

“Alkyl” means a straight or branched chain saturated hydrocarbon containing 1-12 carbon atoms. Examples of a (C₁-C₆)alkyl group include, but are not limited to, methyl, ethyl, propyl, butyl, pentyl, hexyl, isopropyl, isobutyl, sec-butyl, tert-butyl, isopentyl, neopentyl, and isohexyl.

“Alkoxy” means a straight or branched chain saturated hydrocarbon containing 1-12 carbon atoms containing a terminal “O” in the chain, e.g., —O(alkyl). Examples of alkoxy groups include, without limitation, methoxy, ethoxy, propoxy, butoxy, t-butoxy, or pentoxy groups.

“Alkenyl” means a straight or branched chain unsaturated hydrocarbon containing 2-12 carbon atoms. The “alkenyl” group contains at least one double bond in the chain. The double bond of an alkenyl group can be unconjugated or conjugated to another unsaturated group. Examples of alkenyl groups include ethenyl, propenyl, n-butenyl, iso-butenyl, pentenyl, or hexenyl. An alkenyl group can be unsubstituted or substituted and may be straight or branched.

“Alkynyl” means a straight or branched chain unsaturated hydrocarbon containing 2-12 carbon atoms. The “alkynyl” group contains at least one triple bond in the chain. Examples of alkenyl groups include ethynyl, propargyl, n-butynyl, iso-butynyl, pentynyl, or hexynyl. An alkynyl group can be unsubstituted or substituted.

“Alkylene” or “alkylenyl” means a divalent alkyl radical. Any of the above mentioned monovalent alkyl groups may be an alkylene by abstraction of a second hydrogen atom from the alkyl. As herein defined, alkylene may also be a (C₁-C₆)alkylene. An alkylene may further be a (C₁-C₄)alkylene. Typical alkylene groups include, but are not limited to, —CH₂—, —CH(CH₃)—, —C(CH₃)₂—, —CH₂CH₂—, —CH₂CH(CH₃)—, —CH₂C(CH₃)₂—, —CH₂CH₂CH₂—, —CH₂CH₂CH₂CH—, and the like.

“Cycloalkyl” or “carbocyclyl” means a monocyclic or polycyclic saturated or partially unsaturated non-aromatic carbon ring containing 3-18 carbon atoms. Examples of cycloalkyl groups include, without limitations, cyclopropyl, cyclobutyl, cyclopentyl, cyclohexyl, cycloheptanyl, cyclooctanyl, norboranyl, norborenyl, bicyclo[2.2.2]octanyl, or bicyclo[2.2.2]octenyl and derivatives thereof. A (C₃-C₈)cycloalkyl is a cycloalkyl group containing between 3 and 8 carbon atoms. A cycloalkyl group can be fused (e.g., decalin) or bridged (e.g., norbornane).

“Heterocyclyl” or “heterocycloalkyl” means a saturated or partially saturated monocyclic or polycyclic ring containing carbon and at least one heteroatom selected from oxygen, nitrogen, or sulfur (O, N, or S) and wherein there is not delocalized n electrons (aromaticity) shared among the ring carbon or heteroatoms. The heterocycloalkyl ring structure may be substituted by one or more substituents. The substituents can themselves be optionally substituted. Examples of heterocyclyl rings include, but are not limited to, oxetanyl, azetadinyl, tetrahydrofuranyl, tetrahydropyranyl, pyrrolidinyl, oxazolinyl, oxazolidinyl, thiazolinyl, thiazolidinyl, pyranyl, thiopyranyl, tetrahydropyranyl, dioxalinyl, piperidinyl, morpholinyl, thiomorpholinyl, thiomorpholinyl S-oxide, thiomorpholinyl S-dioxide, piperazinyl, azepinyl, oxepinyl, diazepinyl, tropanyl, oxazolidinonyl, 1,4-dioxanyl, dihydrofuranyl, 1,3-dioxolanyl, imidazolidinyl, imidazolinyl, dithiolanyl, and homotropanyl.

“Hydroxyalkyl” means an alkyl group substituted with one or more —OH groups. Examples of hydroxyalkyl groups include HO—CH₂—, HO—CH₂CH₂—, and CH₂—CH(OH)—.

“Haloalkyl” means an alkyl group substituted with one or more halogens. Examples of haloalkyl groups include, but are not limited to, trifluoromethyl, difluoromethyl, pentafluoroethyl, trichloromethyl, etc.

“Haloalkoxy” means an alkoxy group substituted with one or more halogens. Examples of haloalkyl groups include, but are not limited to, trifluoromethoxy, difluoromethoxy, pentafluoroethoxy, trichloromethoxy, etc.

“Cyano” means a substituent having a carbon atom joined to a nitrogen atom by a triple bond, e.g., C≡N.

“Amino” means a substituent containing at least one nitrogen atom (e.g., NH₂).

“Pomalidomide” or 4-amino-2-(2,6-dioxopiperidin-3-yl)isoindoline-1,3-dione has the following structure:

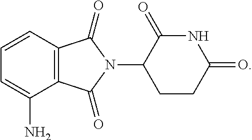 [SMILES: Nc1cccc2c1C(=O)N(C1CCC(=O)NC1=O)C2=O]

### B. Salt, Prodrug, Derivative, and Solvate Terms and Conventions

“Prodrug” or “prodrug derivative” mean a covalently-bonded derivative or carrier of the parent compound or active drug substance which undergoes at least some biotransformation prior to exhibiting its pharmacological effect(s). In general, such prodrugs have metabolically cleavable groups and are rapidly transformed in vivo to yield the parent compound, for example, by hydrolysis in blood, and generally include esters and amide analogs of the parent compounds. The prodrug is formulated with the objectives of improved chemical stability, improved patient acceptance and compliance, improved bioavailability, prolonged duration of action, improved organ selectivity, improved formulation (e.g., increased hydrosolubility), and/or decreased side effects (e.g., toxicity). In general, prodrugs themselves have weak or no biological activity and are stable under ordinary conditions. Prodrugs can be readily prepared from the parent compounds using methods known in the art, such as those described in A Textbook of Drug Design and Development, Krogsgaard-Larsen and H. Bundgaard (eds.), Gordon & Breach, 1991, particularly Chapter 5: “Design and Applications of Prodrugs”; Design of Prodrugs, H. Bundgaard (ed.), Elsevier, 1985; Prodrugs: Topical and Ocular Drug Delivery, K. B. Sloan (ed.), Marcel Dekker, 1998; Methods in Enzymology, K. Widder et al. (eds.), Vol. 42, Academic Press, 1985, particularly pp. 309-396; Burger's Medicinal Chemistry and Drug Discovery, 5th Ed., M. Wolff (ed.), John Wiley & Sons, 1995, particularly Vol. 1 and pp. 172-178 and pp. 949-982; Pro-Drugs as Novel Delivery Systems, T. Higuchi and V. Stella (eds.), Am. Chem. Soc., 1975; Bioreversible Carriers in Drug Design, E. B. Roche (ed.), Elsevier, 1987, each of which is incorporated herein by reference in their entireties.

“Pharmaceutically acceptable prodrug” as used herein means a prodrug of a compound of the disclosure which is, within the scope of sound medical judgment, suitable for use in contact with the tissues of humans and lower animals without undue toxicity, irritation, allergic response, and the like, commensurate with a reasonable benefit/risk ratio, and effective for their intended use, as well as the zwitterionic forms, where possible.

“Salt” means an ionic form of the parent compound or the product of the reaction between the parent compound with a suitable acid or base to make the acid salt or base salt of the parent compound. Salts of the compounds of the present disclosure can be synthesized from the parent compounds which contain a basic or acidic moiety by conventional chemical methods. Generally, the salts are prepared by reacting the free base or acid parent compound with stoichiometric amounts or with an excess of the desired salt-forming inorganic or organic acid or base in a suitable solvent or various combinations of solvents.

“Pharmaceutically acceptable salt” means a salt of a compound of the disclosure which is, within the scope of sound medical judgment, suitable for use in contact with the tissues of humans and lower animals without undue toxicity, irritation, allergic response, and the like, commensurate with a reasonable benefit/risk ratio, generally water or oil-soluble or dispersible, and effective for their intended use. The term includes pharmaceutically-acceptable acid addition salts and pharmaceutically-acceptable base addition salts. As the compounds of the present disclosure are useful in both free base and salt form, in practice, the use of the salt form amounts to use of the base form. Lists of suitable salts are found in, e.g., S. M. Birge et al., J. Pharm. Sci., 1977, 66, pp. 1-19, which is hereby incorporated by reference in its entirety.

“Pharmaceutically-acceptable acid addition salt” means those salts which retain the biological effectiveness and properties of the free bases and which are not biologically or otherwise undesirable, formed with inorganic acids such as hydrochloric acid, hydrobromic acid, hydroiodic acid, sulfuric acid, sulfamic acid, nitric acid, phosphoric acid, and the like, and organic acids such as acetic acid, trichloroacetic acid, trifluoroacetic acid, adipic acid, alginic acid, ascorbic acid, aspartic acid, benzenesulfonic acid, benzoic acid, 2-acetoxybenzoic acid, butyric acid, camphoric acid, camphorsulfonic acid, cinnamic acid, citric acid, digluconic acid, ethanesulfonic acid, glutamic acid, glycolic acid, glycerophosphoric acid, hemisulfic acid, heptanoic acid, hexanoic acid, formic acid, fumaric acid, 2-hydroxyethanesulfonic acid (isethionic acid), lactic acid, maleic acid, hydroxymaleic acid, malic acid, malonic acid, mandelic acid, mesitylenesulfonic acid, methanesulfonic acid, naphthalenesulfonic acid, nicotinic acid, 2-naphthalenesulfonic acid, oxalic acid, pamoic acid, pectinic acid, phenylacetic acid, 3-phenylpropionic acid, picric acid, pivalic acid, propionic acid, pyruvic acid, pyruvic acid, salicylic acid, stearic acid, succinic acid, sulfanilic acid, tartaric acid, p-toluenesulfonic acid, undecanoic acid, and the like.

“Pharmaceutically-acceptable base addition salt” means those salts which retain the biological effectiveness and properties of the free acids and which are not biologically or otherwise undesirable, formed with inorganic bases such as ammonia or hydroxide, carbonate, or bicarbonate of ammonium or a metal cation such as sodium, potassium, lithium, calcium, magnesium, iron, zinc, copper, manganese, aluminum, and the like. Particularly preferred are the ammonium, potassium, sodium, calcium, and magnesium salts. Salts derived from pharmaceutically-acceptable organic nontoxic bases include salts of primary, secondary, and tertiary amines, quaternary amine compounds, substituted amines including naturally occurring substituted amines, cyclic amines and basic ion-exchange resins, such as methylamine, dimethylamine, trimethylamine, ethylamine, diethylamine, triethylamine, isopropylamine, tripropylamine, tributylamine, ethanolamine, diethanolamine, 2-dimethylaminoethanol, 2-diethylaminoethanol, dicyclohexylamine, lysine, arginine, histidine, caffeine, hydrabamine, choline, betaine, ethylenediamine, glucosamine, methylglucamine, theobromine, purines, piperazine, piperidine, N-ethylpiperidine, tetramethylammonium compounds, tetraethylammonium compounds, pyridine, N,N-dimethylaniline, N-methylpiperidine, N-methylmorpholine, dicyclohexylamine, dibenzylamine, N,N-dibenzylphenethylamine, 1-ephenamine, N,N′-dibenzylethylenediamine, polyamine resins, and the like. Particularly preferred organic nontoxic bases are isopropylamine, diethylamine, ethanolamine, trimethylamine, dicyclohexylamine, choline, and caffeine.

“Solvate” means a complex of variable stoichiometry formed by a solute, for example, a compound of Formula (I)) and solvent, for example, water, ethanol, or acetic acid. This physical association may involve varying degrees of ionic and covalent bonding, including hydrogen bonding. In certain instances, the solvate will be capable of isolation, for example, when one or more solvent molecules are incorporated in the crystal lattice of the crystalline solid. In general, such solvents selected for the purpose of the disclosure do not interfere with the biological activity of the solute. Solvates encompasses both solution-phase and isolatable solvates. Representative solvates include hydrates, ethanolates, methanolates, and the like.

“Hydrate” means a solvate wherein the solvent molecule(s) is/are water.

The compounds of the present disclosure as discussed below include the free base or acid thereof, their salts, solvates, and prodrugs and may include oxidized sulfur atoms or quaternized nitrogen atoms in their structure, although not explicitly stated or shown, particularly the pharmaceutically acceptable forms thereof. Such forms, particularly the pharmaceutically acceptable forms, are intended to be embraced by the appended claims.

### C. Isomer Terms and Conventions

“Isomers” means compounds having the same number and kind of atoms, and hence the same molecular weight, but differing with respect to the arrangement or configuration of the atoms in space. The term includes stereoisomers and geometric isomers.

“Stereoisomer” or “optical isomer” mean a stable isomer that has at least one chiral atom or restricted rotation giving rise to perpendicular dissymmetric planes (e.g., certain biphenyls, allenes, and spiro compounds) and can rotate plane-polarized light. Because asymmetric centers and other chemical structure exist in the compounds of the disclosure, which may give rise to stereoisomerism, the disclosure contemplates stereoisomers and mixtures thereof. The compounds of the disclosure and their salts include asymmetric carbon atoms and may therefore exist as single stereoisomers, racemates, and as mixtures of enantiomers and diastereomers. Typically, such compounds will be prepared as a racemic mixture. If desired, however, such compounds can be prepared or isolated as pure stereoisomers, i.e., as individual enantiomers or diastereomers, or as stereoisomer-enriched mixtures. As discussed in more detail below, individual stereoisomers of compounds are prepared by synthesis from optically active starting materials containing the desired chiral centers or by preparation of mixtures of enantiomeric products followed by separation or resolution, such as conversion to a mixture of diastereomers followed by separation or recrystallization, chromatographic techniques, use of chiral resolving agents, or direct separation of the enantiomers on chiral chromatographic columns. Starting compounds of particular stereochemistry are either commercially available or are made by the methods described below and resolved by techniques well-known in the art.

“Enantiomers” means a pair of stereoisomers that are non-superimposable mirror images of each other.

“Diastereoisomers” or “diastereomers” mean optical isomers which are not mirror images of each other.

“Racemic mixture” or “racemate” mean a mixture containing equal parts of individual enantiomers.

“Non-racemic mixture” means a mixture containing unequal parts of individual enantiomers.

“Geometrical isomer” means a stable isomer, which results from restricted freedom of rotation about double bonds (e.g., cis-2-butene and trans-2-butene) or in a cyclic structure (e.g., cis-1,3-dichlorocyclobutane and trans-1,3-dichlorocyclobutane). Because carbon-carbon double (olefinic) bonds, C═N double bonds, cyclic structures, and the like may be present in the compounds of the disclosure, the disclosure contemplates each of the various stable geometric isomers and mixtures thereof resulting from the arrangement of substituents around these double bonds and in these cyclic structures. The substituents and the isomers are designated using the cis/trans convention or using the E or Z system, wherein the term “E” means higher order substituents on opposite sides of the double bond, and the term “Z” means higher order substituents on the same side of the double bond. A thorough discussion of E and Z isomerism is provided in J. March, Advanced Organic Chemistry: Reactions, Mechanisms, and Structure, 4th ed., John Wiley & Sons, 1992, which is hereby incorporated by reference in its entirety. Several of the following examples represent single E isomers, single Z isomers, and mixtures of E/Z isomers. Determination of the E and Z isomers can be done by analytical methods such as x-ray crystallography, ¹H NMR, and ¹³C NMR.

Some of the compounds of the disclosure can exist in more than one tautomeric form. As mentioned above, the compounds of the disclosure include all such tautomers.

It is well-known in the art that the biological and pharmacological activity of a compound is sensitive to the stereochemistry of the compound. Thus, for example, enantiomers often exhibit strikingly different biological activity including differences in pharmacokinetic properties, including metabolism, protein binding, and the like, and pharmacological properties, including the type of activity displayed, the degree of activity, toxicity, and the like. Thus, one skilled in the art will appreciate that one enantiomer may be more active or may exhibit beneficial effects when enriched relative to the other enantiomer or when separated from the other enantiomer. Additionally, one skilled in the art would know how to separate, enrich, or selectively prepare the enantiomers of the compounds of the disclosure from this disclosure and the knowledge of the prior art.

Thus, although the racemic form of drug may be used, it is often less effective than administering an equal amount of enantiomerically pure drug; indeed, in some cases, one enantiomer may be pharmacologically inactive and would merely serve as a simple diluent. For example, although ibuprofen had been previously administered as a racemate, it has been shown that only the S-isomer of ibuprofen is effective as an anti-inflammatory agent (in the case of ibuprofen, however, although the R-isomer is inactive, it is converted in vivo to the S-isomer, thus, the rapidity of action of the racemic form of the drug is less than that of the pure S-isomer). Furthermore, the pharmacological activities of enantiomers may have distinct biological activity. For example, S-penicillamine is a therapeutic agent for chronic arthritis, while R-penicillamine is toxic. Indeed, some purified enantiomers have advantages over the racemates, as it has been reported that purified individual isomers have faster transdermal penetration rates compared to the racemic mixture. See U.S. Pat. Nos. 5,114,946 and 4,818,541.

Thus, if one enantiomer is pharmacologically more active, less toxic, or has a preferred disposition in the body than the other enantiomer, it would be therapeutically more beneficial to administer that enantiomer preferentially. In this way, the patient undergoing treatment would be exposed to a lower total dose of the drug and to a lower dose of an enantiomer that is possibly toxic or an inhibitor of the other enantiomer.

Preparation of pure enantiomers or mixtures of desired enantiomeric excess (ee) or enantiomeric purity are accomplished by one or more of the many methods of (a) separation or resolution of enantiomers, or (b) enantioselective synthesis known to those of skill in the art, or a combination thereof. These resolution methods generally rely on chiral recognition and include, for example, chromatography using chiral stationary phases, enantioselective host-guest complexation, resolution or synthesis using chiral auxiliaries, enantioselective synthesis, enzymatic and nonenzymatic kinetic resolution, or spontaneous enantioselective crystallization.

Such methods are disclosed generally in Chiral Separation Techniques: A Practical Approach (2nd Ed.), G. Subramanian (ed.), Wiley-VCH, 2000; T. E. Beesley and R. P. W. Scott, Chiral Chromatography, John Wiley & Sons, 1999; and Satinder Ahuja, Chiral Separations by Chromatography, Am. Chem. Soc., 2000. Furthermore, there are equally well-known methods for the quantitation of enantiomeric excess or purity, for example, GC, HPLC, CE, or NMR, and assignment of absolute configuration and conformation, for example, CD ORD, X-ray crystallography, or NMR.

In general, all tautomeric forms and isomeric forms and mixtures, whether individual geometric isomers or stereoisomers or racemic or non-racemic mixtures, of a chemical structure or compound is intended, unless the specific stereochemistry or isomeric form is specifically indicated in the compound name or structure.

### D. Pharmaceutical Administration and Treatment Terms and Conventions

A “patient” or “subject” is a mammal, e.g., a human, mouse, rat, guinea pig, dog, cat, horse, cow, pig, or nonhuman primate, such as a monkey, chimpanzee, baboon or, rhesus. In certain embodiments, the subject is a primate. In yet other embodiments, the subject is a human.

An “effective amount” or “therapeutically effective amount” when used in connection with a compound means an amount of a compound of the present disclosure that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The terms “pharmaceutically effective amount” or “therapeutically effective amount” means an amount of a compound according to the disclosure which, when administered to a patient in need thereof, is sufficient to effect treatment for disease-states, conditions, or disorders for which the compounds have utility. Such an amount would be sufficient to elicit the biological or medical response of a tissue, system, or patient that is sought by a researcher or clinician. The amount of a compound of according to the disclosure which constitutes a therapeutically effective amount will vary depending on such factors as the compound and its biological activity, the composition used for administration, the time of administration, the route of administration, the rate of excretion of the compound, the duration of treatment, the type of disease-state or disorder being treated and its severity, drugs used in combination with or coincidentally with the compounds of the disclosure, and the age, body weight, general health, sex, and diet of the patient.

Such a therapeutically effective amount can be determined routinely by one of ordinary skill in the art having regard to their own knowledge, the prior art, and this disclosure.

As used herein, the term “pharmaceutical composition” refers to a compound of the disclosure, or a pharmaceutically acceptable salt, hydrate, solvate, prodrug, stereoisomer, or tautomer thereof, together with at least one pharmaceutically acceptable carrier, in a form suitable for oral or parenteral administration.

“Carrier” encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

A subject is “in need of” a treatment if such subject would benefit biologically, medically, or in quality of life from such treatment (preferably, a human).

As used herein, the term “inhibit”, “inhibition”, or “inhibiting” refers to the reduction or suppression of a given condition, symptom, or disorder, or disease, or a significant decrease in the baseline activity of a biological activity or process.

As used herein, the term “treat”, “treating”, or “treatment” of any disease or disorder refers to alleviating or ameliorating the disease or disorder (i.e., slowing or arresting the development of the disease or at least one of the clinical symptoms thereof); or alleviating or ameliorating at least one physical parameter or biomarker associated with the disease or disorder, including those which may not be discernible to the patient.

As used herein, the term “prevent”, “preventing”, or “prevention” of any disease or disorder refers to the prophylactic treatment of the disease or disorder; or delaying the onset or progression of the disease or disorder.

“Pharmaceutically acceptable” means that the substance or composition must be compatible chemically and/or toxicologically, with the other ingredients comprising a formulation, and/or the mammal being treated therewith.

“Disorder” means, and is used interchangeably with, the terms disease, condition, or illness, unless otherwise indicated.

“Administer”, “administering”, or “administration” means to either directly administering a disclosed compound or pharmaceutically acceptable salt of the disclosed compound or a composition to a subject, or administering a prodrug derivative or analog of the compound or pharmaceutically acceptable salt of the compound or composition to the subject, which can form an equivalent amount of active compound within the subject's body.

“Prodrug” means a compound which is convertible in vivo by metabolic means (e.g., by hydrolysis) to a disclosed compound.

“Compounds of the present disclosure”, “compounds of the disclosure”, and equivalent expressions (unless specifically identified otherwise) refer to compounds of Formulae (I), (Ia), and (Ib), and compounds (I-1), (I-2), (I-3), (I-4), (I-5), (I-6), (I-7), (I-8), (I-9), (I-10), (I-11), (I-12), (I-13), (I-14), (I-15), (I-16), (I-17), (I-18), (I-24), (I-25), (I-26), (I-27), (I-28), (I-29), (I-30), (I-31), (I-32), (I-33), (I-34), (I-34), (I-36), (I-37), and (I-38), as herein described including the tautomers, the prodrugs, salts particularly the pharmaceutically acceptable salts, and the solvates and hydrates thereof, where the context so permits thereof, as well as all stereoisomers (including diastereoisomers and enantiomers), rotamers, tautomers, and isotopically labelled compounds (including deuterium substitutions), as well as inherently formed moieties (e.g., polymorphs, solvates and/or hydrates). For purposes of this disclosure, solvates and hydrates are generally considered compositions. In general and preferably, the compounds of the disclosure and the formulas designating the compounds of the disclosure are understood to only include the stable compounds thereof and exclude unstable compounds, even if an unstable compound might be considered to be literally embraced by the compound formula. Similarly, reference to intermediates, whether or not they themselves are claimed, is meant to embrace their salts and solvates, where the context so permits. For the sake of clarity, particular instances when the context so permits are sometimes indicated in the text, but these instances are purely illustrative and it is not intended to exclude other instances when the context so permits.

“Stable compound” or “stable structure” means a compound that is sufficiently robust to survive isolation to a useful degree of purity from a reaction mixture, and formulation into an efficacious therapeutic or diagnostic agent. For example, a compound, which would have a “dangling valency” or is a carbanion is not a compound contemplated by the disclosure.

In a specific embodiment, the term “about” or “approximately” means within 20%, preferably within 10%, and more preferably within 5% of a given value or range.

The yield of each of the reactions described herein is expressed as a percentage of the theoretical yield. “Cancer” means any cancer caused by the proliferation of malignant neoplastic cells, such as tumors, neoplasms, carcinomas, sarcomas, leukemias, lymphomas, and the like. For example, cancers include, but are not limited to, mesothelioma, leukemias, and lymphomas such as cutaneous T-cell lymphomas (CTCL), noncutaneous peripheral T-cell lymphomas, lymphomas associated with human T-cell lymphotrophic virus (HTLV) such as adult T-cell leukemia/lymphoma (ATLL), B-cell lymphoma, acute nonlymphocytic leukemias, chronic lymphocytic leukemia, chronic myelogenous leukemia, acute myelogenous leukemia, lymphomas, and multiple myeloma, non-Hodgkin lymphoma, acute lymphatic leukemia (ALL), chronic lymphatic leukemia (CLL), Hodgkin's lymphoma, Burkitt lymphoma, adult T-cell leukemia lymphoma, acute-myeloid leukemia (AML), chronic myeloid leukemia (CML), or hepatocellular carcinoma. Further examples include myelodisplastic syndrome, childhood solid tumors such as brain tumors, neuroblastoma, retinoblastoma, Wilms' tumor, bone tumors, and soft-tissue sarcomas, common solid tumors of adults such as head and neck cancers (e.g., oral, laryngeal, and nasopharyngeal), esophageal cancer, genitourinary cancers (e.g., prostate, bladder, renal, uterine, ovarian, testicular), lung cancer (e.g., small-cell and non-small cell), breast cancer, pancreatic cancer, melanoma, and other skin cancers, stomach cancer, brain tumors, tumors related to Gorlin's syndrome (e.g., medulloblastoma, meningioma, etc.), and liver cancer. Additional exemplary forms of cancer which may be treated by the subject compounds include, but are not limited to, cancer of skeletal or smooth muscle, stomach cancer, cancer of the small intestine, rectum carcinoma, cancer of the salivary gland, endometrial cancer, adrenal cancer, anal cancer, rectal cancer, parathyroid cancer, and pituitary cancer.

Additional cancers that the compounds described herein may be useful in preventing, treating, and studying are, for example, colon carcinoma, familiary adenomatous polyposis carcinoma, and hereditary non-polyposis colorectal cancer, or melanoma. Further, cancers include, but are not limited to, labial carcinoma, larynx carcinoma, hypopharynx carcinoma, tongue carcinoma, salivary gland carcinoma, gastric carcinoma, adenocarcinoma, thyroid cancer (medullary and papillary thyroid carcinoma), renal carcinoma, kidney parenchyma carcinoma, cervix carcinoma, uterine corpus carcinoma, endometrium carcinoma, chorion carcinoma, testis carcinoma, urinary carcinoma, melanoma, brain tumors such as glioblastoma, astrocytoma, meningioma, medulloblastoma and peripheral neuroectodermal tumors, gall bladder carcinoma, bronchial carcinoma, multiple myeloma, basalioma, teratoma, retinoblastoma, choroidea melanoma, seminoma, rhabdomyosarcoma, craniopharyngeoma, osteosarcoma, chondrosarcoma, myosarcoma, liposarcoma, fibrosarcoma, Ewing's sarcoma, and plasmocytoma.

“Simultaneously” or “simultaneous” when referring to a method of treating or a therapeutic use means with a combination of a compound of Formula (I) or Embodiment 16, 17, or 35, or a pharmaceutically acceptable salt, hydrate, solvate, prodrug, stereoisomer, or tautomer thereof, and one or more second agent(s) means administration of the compound and the one or more second agent(s) by the same route and at the same time.

“Separately” or “separate” when referring to a method of treating or a therapeutic use means with a combination of a compound of Formula (I) or Embodiment 16, 17, or 35, or a pharmaceutically acceptable salt, hydrate, solvate, prodrug, stereoisomer, or tautomer thereof, and one or more second agent(s) means administration of the compound and the one or more second agent(s) by different routes and at approximately the same time.

By therapeutic administration “over a period of time” means, when referring to a method of treating or a therapeutic use with a combination of a compound of Formula (I) or Embodiment 16, 17, or 35, or a pharmaceutically acceptable salt, hydrate, solvate, prodrug, stereoisomer, or tautomer thereof, and one or more second agent(s), administration of the compound and the one or more second agent(s) by the same or different routes and at different times. In some embodiments, the administration of the compound or the one or more second agent(s) occurs before the administration of the other begins. In this way, it is possible to administer a one of the active ingredients (i.e., a compound of Formula (I) or Embodiment 16, 17, or 35, or a pharmaceutically acceptable salt, hydrate, solvate, prodrug, stereoisomer, or tautomer thereof, or one or more second agent(s)) for several months before administering the other active ingredient or ingredients. In this case, no simultaneous administration occurs. Another therapeutic administration over a period of time consists of the administration over time of the two or more active ingredients of the combination using different frequencies of administration for each of the active ingredients, whereby at certain time points in time simultaneous administration of all of the active ingredients takes place whereas at other time points in time only a part of the active ingredients of the combination may be administered (e.g., for example, a compound of Formula (I) or Embodiment 16, 17, or 35, or a pharmaceutically acceptable salt, hydrate, solvate, prodrug, stereoisomer, or tautomer thereof, and the one or more second agents the therapeutic administration over a period of time could be such that a compound of Formula (I) or Embodiment 16, 17, or 35, or a pharmaceutically acceptable salt, hydrate, solvate, prodrug, stereoisomer, or tautomer thereof, is administered once a day and the one or more second agent(s) is administered once every four weeks.)

“IKZF2-dependent disease or disorder” means any disease or disorder which is directly or indirectly affected by the modulation of IKZF2 protein levels.

“IKZF4-dependent disease or disorder” means any disease or disorder which is directly or indirectly affected by the modulation of IKZF4 protein levels.

### D. Specific Embodiments and Methods for Testing Compounds of Formula (I)

The present disclosure relates to compounds or pharmaceutically acceptable salts, hydrates, solvates, prodrugs, stereoisomers, or tautomers thereof, capable of modulating IKZF2 protein levels, which are useful for the treatment of diseases and disorders associated with modulation of IKZF2 protein levels. The disclosure further relates to compounds, or pharmaceutically acceptable salts, hydrates, solvates, prodrugs, stereoisomers, or tautomers thereof, which are useful for reducing or decreasing IKZF2 protein levels.

In one embodiment, the compounds of Formula (I) have the structure of Formula (Ia):

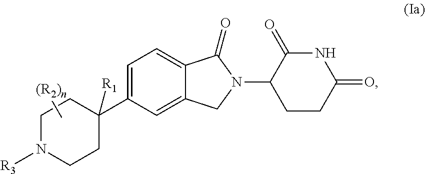

or pharmaceutically acceptable salts, hydrates, solvates, prodrugs, stereoisomers, and tautomers thereof.

In another embodiment, the compounds of Formula (I) have the structure of Formula (Ib):

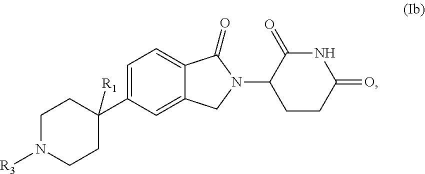

or pharmaceutically acceptable salts, hydrates, solvates, prodrugs, stereoisomers, and tautomers thereof.

In some embodiments of the formulae above (i.e., Formula (I), Formula (Ia), and/or Formula (Ib)),
R₃ is (C₁-C₆)alkyl, (C₆-C₁₀)aryl, 5- or 6-membered heteroaryl comprising 1 to 3 heteroatoms selected from O, N, and S, (C₃-C₈)cycloalkyl, or 4- to 7-membered heterocycloalkyl comprising 1 to 3 heteroatoms selected from O, N, and S, wherein the alkyl is optionally substituted with one to four R₄; and the aryl, heteroaryl, cycloalkyl, and heterocycloalkyl are optionally substituted with one to four R₅, or
each R₄ is independently selected from —C(O)OR₆, —C(O)NR₆R₆′, —NR₆C(O)R₆′, halogen, —OH, —NH₂, CN, (C₆-C₁₀)aryl, 5- or 6-membered heteroaryl comprising 1 to 4 heteroatoms selected from O, N, and S, (C₃-C₈)cycloalkyl, and 4- to 7-membered heterocycloalkyl ring comprising 1 to 3 heteroatoms selected from O, N, and S, wherein the aryl, heteroaryl, cycloalkyl, and heterocycloalkyl groups are optionally substituted with one to four R₇;
each R₅ is independently selected from (C₁-C₆)alkyl, (C₂-C₆)alkenyl, (C₂-C₆)alkynyl, (C₁-C₆)alkoxy, (C₁-C₆)haloalkyl, (C₁-C₆)haloalkoxy, (C₁-C₆)hydroxyalkyl, halogen, —OH, —NH₂, CN, (C₃-C₇)cycloalkyl, 5- to 7-membered heterocycloalkyl comprising 1 to 3 heteroatoms selected from O, N, and S, (C₆-C₁₀)aryl, and 5- or 6-membered heteroaryl comprising 1 to 3 heteroatoms selected from O, N, and S, or
two R₅, when on adjacent atoms, together with the atoms to which they are attached form a (C₆-C₁₀)aryl ring or a 5- or 6-membered heteroaryl ring comprising 1 to 3 heteroatoms selected from O, N, and S, optionally substituted with one to four R₁₀, or
two R₅ together with the atoms to which they are attached form a (C₃-C₇)cycloalkyl ring or a 4- to 7-membered heterocycloalkyl ring comprising 1 to 3 heteroatoms selected from O, N, and S optionally substituted with one to four R₁₀;
each R₇ is independently selected from (C₁-C₆)alkyl, (C₂-C₆)alkenyl, (C₂-C₆)alkynyl, (C₁-C₆)alkoxy, (C₁-C₆)haloalkyl, (C₁-C₆)haloalkoxy, —C(O)R₈, —(CH₂)₀₋₃C(O)OR₈, —C(O)NR₈R₉, —NR₈C(O)R₉, —NR₈C(O)OR₉, —S(O)ₚNR₈R₉, —S(O)ₚR₁₂, (C₁-C₆)hydroxyalkyl, halogen, —OH, —O(CH₂)₁₋₃CN, —NH₂, CN, —O(CH₂)₀₋₃(C₆-C₁₀)aryl, adamantyl, —O(CH₂)₀₋₃-5- or 6-membered heteroaryl comprising 1 to 3 heteroatoms selected from O, N, and S, (C₆-C₁₀)aryl, monocyclic or bicyclic 5- to 10-membered heteroaryl comprising 1 to 3 heteroatoms selected from O, N, and S, (C₃-C₇)cycloalkyl, and 5- to 7-membered heterocycloalkyl comprising 1 to 3 heteroatoms selected from O, N, and S, wherein the alkyl is optionally substituted with one to four R₁, and the aryl, heteroaryl, and heterocycloalkyl are optionally substituted with one to four substituents each independently selected from halogen, (C₁-C₆)alkyl, (C₁-C₆)haloalkyl, and (C₁-C₆)alkoxy, or
two R₇ together with the carbon atom to which they are attached form a ═(O), or
two R₇, when on adjacent atoms, together with the atoms to which they are attached form a (C₆-C₁₀)aryl ring or a 5- or 6-membered heteroaryl ring comprising 1 to 3 heteroatoms selected from O, N, and S, optionally substituted with one to four R₁₀, or
two R₇ together with the atoms to which they are attached form a (C₅-C₇) cycloalkyl ring or a 5- to 7-membered heterocycloalkyl ring comprising 1 to 3 heteroatoms selected from O, N, and S, optionally substituted with one to four R₁₀;
each R₁₁ is independently selected from CN, (C₁-C₆)alkoxy, (C₆-C₁₀)aryl, and 5- to 7-membered heterocycloalkyl comprising 1 to 3 heteroatoms selected from O, N, and S, wherein the aryl and heterocycloalkyl are optionally substituted with one to four substituents each independently selected from (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —NH₂, and CN;
or a pharmaceutically acceptable salt, hydrate, solvate, prodrug, stereoisomer, or tautomer thereof.

In some embodiments of the formulae above, Rₓ is D. In another embodiment, Rₓ is H.

In some embodiments of the formulae above, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN. In another embodiment, R₁ is —C(O)NH₂, —C(O)OH, or CN. In yet another embodiment, R₁ is (C₁-C₆)alkyl, (C₁-C₆)alkoxy, (C₁-C₆)haloalkyl, (C₁-C₆)haloalkoxy, (C₁-C₆)hydroxyalkyl, or halogen. In another embodiment, R₁ is —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH, or CN. In yet another embodiment, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, or CN. In another embodiment, R₁ is (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, or CN. In yet another embodiment, R₁ is (C₁-C₆)alkoxy, (C₁-C₆)hydroxyalkyl, halogen, —OH, —(CH₂)₀₋₂NH₂, or CN. In another embodiment, R₁ is (C₁-C₆)alkoxy, halogen, —OH, —(CH₂)₀₋₂NH₂, or CN. In yet another embodiment, R₁ is (C₁-C₆)alkoxy, halogen, —OH, —NH₂, or CN.

In some embodiments of the formulae above, each R₂ is independently (C₁-C₆)haloalkyl, (C₁-C₆)hydroxyalkyl, CN, or halogen. In another embodiment, each R₂ is independently (C₁-C₆)alkyl, (C₁-C₆)haloalkyl, CN, or halogen. In yet another embodiment, each R₂ is independently (C₁-C₆)alkyl, (C₁-C₆)hydroxyalkyl, CN, or halogen. In another embodiment, each R₂ is independently (C₁-C₆)alkyl, (C₁-C₆)haloalkyl, CN, or halogen. In yet another embodiment, each R₂ is independently (C₁-C₆)alkyl or (C₁-C₆)haloalkyl.

In another embodiment, each R₂ is independently (C₁-C₆)haloalkyl, (C₁-C₆)hydroxyalkyl, or halogen. In another embodiment, each R₂ is independently (C₁-C₆)alkyl, (C₁-C₆)haloalkyl, or halogen. In yet another embodiment, each R₂ is independently (C₁-C₆)alkyl, (C₁-C₆)hydroxyalkyl, or halogen. In another embodiment, each R₂ is independently (C₁-C₆)alkyl, (C₁-C₆)haloalkyl, or halogen. In yet another embodiment, each R₂ is independently (C₁-C₆)alkyl or (C₁-C₆)haloalkyl. In another embodiment, each R₂ is independently (C₁-C₆)alkyl or halogen. In yet another embodiment, each R₂ is independently (C₁-C₆)haloalkyl or halogen. In another embodiment, each R₂ is independently (C₁-C₆)alkyl.

In some embodiments of the formulae above, R₁ and R₂ together with the carbon atoms to which they are attached form a (C₃-C₇)cycloalkyl or a 4- to 6-membered heterocycloalkyl ring comprising 1 to 3 heteroatoms selected from O, N, and S. In another embodiment, R₁ and R₂ together with the carbon atoms to which they are attached form a (C₃-C₇)cycloalkyl or a 5- or 6-membered heterocycloalkyl ring comprising 1 to 3 heteroatoms selected from O, N, and S. In yet another embodiment, R₁ and R₂ together with the carbon atoms to which they are attached form a (C₃-C₇)cycloalkyl or a 4- or 5-membered heterocycloalkyl ring comprising 1 to 3 heteroatoms selected from O, N, and S. In another embodiment, R₁ and R₂ together with the carbon atoms to which they are attached form a (C₄-C₇)cycloalkyl or a 4- to 6-membered heterocycloalkyl ring comprising 1 to 3 heteroatoms selected from O, N, and S. In yet another embodiment, R₁ and R₂ together with the carbon atoms to which they are attached form a (C₄-C₆)cycloalkyl or a 4- to 6-membered heterocycloalkyl ring comprising 1 to 3 heteroatoms selected from O, N, and S.

In another embodiment, R₁ and R₂ together with the carbon atoms to which they are attached form a (C₃-C₇)cycloalkyl. In yet another embodiment, R₁ and R₂ together with the carbon atoms to which they are attached form a (C₃-C₆)cycloalkyl. In another embodiment, R₁ and R₂ together with the carbon atoms to which they are attached form a (C₄-C₇)cycloalkyl. In yet another embodiment, R₁ and R₂ together with the carbon atoms to which they are attached form a (C₅-C₇)cycloalkyl. In another embodiment, R₁ and R₂ together with the carbon atoms to which they are attached form a (C₆-C₇)cycloalkyl. In yet another embodiment, R₁ and R₂ together with the carbon atoms to which they are attached form a (C₅-C₆)cycloalkyl. In another embodiment, R₁ and R₂ together with the carbon atoms to which they are attached form a (C₄-C₆)cycloalkyl. In yet another embodiment, R₁ and R₂ together with the carbon atoms to which they are attached form a (C₃-C₆)cycloalkyl. In another embodiment, R₁ and R₂ together with the carbon atoms to which they are attached form a (C₃-C₅)cycloalkyl. In yet another embodiment, R₁ and R₂ together with the carbon atoms to which they are attached form a 4- to 6-membered heterocycloalkyl ring comprising 1 to 3 heteroatoms selected from O, N, and S. In another embodiment, R₁ and R₂ together with the carbon atoms to which they are attached form a 5- or 6-membered heterocycloalkyl ring comprising 1 to 3 heteroatoms selected from O, N, and S. In yet another embodiment, R₁ and R₂ together with the carbon atoms to which they are attached form a 4- or 5-membered heterocycloalkyl ring comprising 1 to 3 heteroatoms selected from O, N, and S.

In some embodiments of the formulae above, two R₂ together with the carbon atoms to which they are attached form a (C₃-C₇)cycloalkyl or a 4- to 6-membered heterocycloalkyl ring comprising 1 to 3 heteroatoms selected from O, N, and S. In another embodiment, two R₂ together with the carbon atoms to which they are attached form a (C₃-C₇)cycloalkyl or a 5- or 6-membered heterocycloalkyl ring comprising 1 to 3 heteroatoms selected from O, N, and S. In yet another embodiment, two R₂ together with the carbon atoms to which they are attached form a (C₃-C₇)cycloalkyl or a 4- or 5-membered heterocycloalkyl ring comprising 1 to 3 heteroatoms selected from O, N, and S. In another embodiment, two R₂ together with the carbon atoms to which they are attached form a (C₄-C₇)cycloalkyl or a 4- to 6-membered heterocycloalkyl ring comprising 1 to 3 heteroatoms selected from O, N, and S. In yet another embodiment, two R₂ together with the carbon atoms to which they are attached form a (C₄-C₆)cycloalkyl or a 4- to 6-membered heterocycloalkyl ring comprising 1 to 3 heteroatoms selected from O, N, and S.

In another embodiment, two R₂ together with the carbon atoms to which they are attached form a (C₃-C₇)cycloalkyl. In yet another embodiment, two R₂ together with the carbon atoms to which they are attached form a (C₃-C₆)cycloalkyl. In another embodiment, two R₂ together with the carbon atoms to which they are attached form a (C₄-C₇)cycloalkyl. In yet another embodiment, two R₂ together with the carbon atoms to which they are attached form a (C₅-C₇)cycloalkyl. In another embodiment, two R₂ together with the carbon atoms to which they are attached form a (C₆-C₇)cycloalkyl. In yet another embodiment, two R₂ together with the carbon atoms to which they are attached form a (C₅-C₆)cycloalkyl. In another embodiment, two R₂ together with the carbon atoms to which they are attached form a (C₄-C₆)cycloalkyl. In yet another embodiment, two R₂ together with the carbon atoms to which they are attached form a (C₃-C₆)cycloalkyl. In another embodiment, two R₂ together with the carbon atoms to which they are attached form a (C₃-C₅)cycloalkyl. In yet another embodiment, two R₂ together with the carbon atoms to which they are attached form a 4- to 6-membered heterocycloalkyl ring comprising 1 to 3 heteroatoms selected from O, N, and S. In another embodiment, two R₂ together with the carbon atoms to which they are attached form a 5- or 6-membered heterocycloalkyl ring comprising 1 to 3 heteroatoms selected from O, N, and S. In yet another embodiment, two R₂ together with the carbon atoms to which they are attached form a 4- or 5-membered heterocycloalkyl ring comprising 1 to 3 heteroatoms selected from O, N, and S.

In some embodiments of the formulae above, R₃ is (C₁-C₆)alkyl, (C₆-C₁₀)aryl, 5- or 6-membered heteroaryl comprising 1 to 3 heteroatoms selected from O, N, and S, (C₃-C₈)cycloalkyl, or 4- to 7-membered heterocycloalkyl comprising 1 to 3 heteroatoms selected from O, N, and S, wherein the alkyl is optionally substituted with one to four R₄; and the aryl, heteroaryl, cycloalkyl, and heterocycloalkyl are optionally substituted with one to four R₅. In another embodiment, R₃ is (C₁-C₆)alkyl, (C₆-C₁₀)aryl, 5- or 6-membered heteroaryl comprising 1 to 3 heteroatoms selected from O, N, and S, (C₃-C₈)cycloalkyl, or 5- to 7-membered heterocycloalkyl comprising 1 to 3 heteroatoms selected from O, N, and S, wherein the alkyl is optionally substituted with one to four R₄; and the aryl, heteroaryl, cycloalkyl, and heterocycloalkyl are optionally substituted with one to four R₅.

In another embodiment, R₃ is (C₁-C₄)alkyl, (C₆-C₁₀)aryl, (C₃-C₈)cycloalkyl, or 5- to 7-membered heterocycloalkyl comprising 1 to 3 heteroatoms selected from O, N, and S, wherein the alkyl is optionally substituted with one to three R₄; and wherein the aryl, cycloalkyl, and heterocycloalkyl are optionally substituted with one to three R₅. In yet another embodiment, R₃ is (C₁-C₄)alkyl, 5- or 6-membered heteroaryl comprising 1 to 3 heteroatoms selected from O, N, and S, (C₃-C₈)cycloalkyl, or 5- to 7-membered heterocycloalkyl comprising 1 to 3 heteroatoms selected from O, N, and S, wherein the alkyl is optionally substituted with one to three R₄; and wherein the heteroaryl, cycloalkyl, and heterocycloalkyl are optionally substituted with one to three R₅. In another embodiment, R₃ is (C₁-C₄)alkyl, (C₆-C₁₀)aryl, 5- or 6-membered heteroaryl comprising 1 to 3 heteroatoms selected from O, N, and S, or (C₃-C₈)cycloalkyl, wherein the alkyl is optionally substituted with one to three R₄; and wherein the aryl, heteroaryl, and cycloalkyl, are optionally substituted with one to three R₅. In another embodiment, R₃ is (C₁-C₄)alkyl, (C₆-C₁₀)aryl, 5- or 6-membered heteroaryl comprising 1 to 3 heteroatoms selected from O, N, and S, or 5- to 7-membered heterocycloalkyl comprising 1 to 3 heteroatoms selected from O, N, and S, wherein the alkyl is optionally substituted with one to three R₄; and wherein the aryl, heteroaryl, and heterocycloalkyl are optionally substituted with one to three R₅.

In another embodiment, R₃ is (C₆-C₁₀)aryl, 5- or 6-membered heteroaryl comprising 1 to 3 heteroatoms selected from O, N, and S, (C₃-C₈)cycloalkyl, or 5- to 7-membered heterocycloalkyl comprising 1 to 3 heteroatoms selected from O, N, and S, wherein the aryl, heteroaryl, cycloalkyl, and heterocycloalkyl are optionally substituted with one to three R₅. In another embodiment, R₃ is (C₆-C₁₀)aryl, (C₃-C₈)cycloalkyl, or 5- to 7-membered heterocycloalkyl comprising 1 to 3 heteroatoms selected from O, N, and S, wherein the aryl, cycloalkyl, and heterocycloalkyl are optionally substituted with one to three R₅. In yet another embodiment, R₃ is phenyl, (C₃-C₈)cycloalkyl, or 5- to 7-membered heterocycloalkyl comprising 1 to 3 heteroatoms selected from O, N, and S, wherein the phenyl, cycloalkyl, and heterocycloalkyl are optionally substituted with one to three R₅. In another embodiment, R₃ is (C₁-C₃)alkyl optionally substituted with one to three R₄. In yet another embodiment, R₃ is (C₁-C₃)alkyl substituted with one to three R₄.

In another embodiment, R₃ is (C₃-C₈)cycloalkyl or 5- to 7-membered heterocycloalkyl comprising 1 to 3 heteroatoms selected from O, N, and S, wherein the cycloalkyl and heterocycloalkyl are optionally substituted with one to three R₅. In yet another embodiment, R₃ is (C₆-C₁₀)aryl or 5- or 6-membered heteroaryl comprising 1 to 3 heteroatoms selected from O, N, and S, wherein the aryl and heteroaryl are optionally substituted with one to three R₅. In another embodiment, R₃ is (C₃-C₈)cycloalkyl or (C₆-C₁₀)aryl, wherein the cycloalkyl and aryl are optionally substituted with one to three R₅. In yet another embodiment, R₃ is 5- or 6-membered heteroaryl comprising 1 to 3 heteroatoms selected from O, N, and S, or 5- to 7-membered heterocycloalkyl comprising 1 to 3 heteroatoms selected from O, N, and S, wherein the heteroaryl and heterocycloalkyl are optionally substituted with one to three R₅. In another embodiment, R₃ is (C₆-C₁₀)aryl optionally substituted with one to three R₅. In yet another embodiment, R₃ is 5- or 6-membered heteroaryl comprising 1 to 3 heteroatoms selected from O, N, and S, optionally substituted with one to three R₅. In another embodiment, R₃ is (C₃-C₈)cycloalkyl optionally substituted with one to three R₅. In yet another embodiment, R₃ is 5- to 7-membered heterocycloalkyl comprising 1 to 3 heteroatoms selected from O, N, and S, optionally substituted with one to three R₅.

In some embodiments of the formulae above, each R₄ is independently selected from —C(O)OR₆, —C(O)NR₆R₆′, —NR₆C(O)R₆′, halogen, —OH, —NH₂, CN, (C₆-C₁₀)aryl, 5- or 6-membered heteroaryl comprising 1 to 4 heteroatoms selected from O, N, and S, (C₃-C₈)cycloalkyl, and 4- to 7-membered heterocycloalkyl ring comprising 1 to 3 heteroatoms selected from O, N, and S, wherein the aryl, heteroaryl, cycloalkyl, and heterocycloalkyl groups are optionally substituted with one to four R₇. In another embodiment, each R₄ is independently selected from —C(O)OR₆, —C(O)NR₆R₆′, —NR₆C(O)R₆′, halogen, —OH, —NH₂, CN, (C₆-C₁₀)aryl, 5- or 6-membered heteroaryl comprising 1 to 4 heteroatoms selected from O, N, and S, (C₃-C₈)cycloalkyl, and 5- to 7-membered heterocycloalkyl ring comprising 1 to 3 heteroatoms selected from O, N, and S, wherein the aryl, heteroaryl, cycloalkyl, and heterocycloalkyl groups are optionally substituted with one to four R₇.

In another embodiment, each R₄ is independently selected from —C(O)OR₆, —C(O)NR₆R₆′, —NR₆C(O)R₆′, halogen, —OH, —NH₂, or CN. In another embodiment, each R₄ is independently selected from —C(O)OR₆, —C(O)NR₆R₆′, —NR₆C(O)R₆′, halogen, or —OH. In another embodiment, each R₄ is independently selected from halogen, —OH, (C₆-C₁₀)aryl, 5- or 6-membered heteroaryl comprising 1 to 4 heteroatoms selected from O, N, and S, (C₃-C₈)cycloalkyl, and 5- to 7-membered heterocycloalkyl ring comprising 1 to 3 heteroatoms selected from O, N, and S, wherein the aryl, heteroaryl, cycloalkyl, and heterocycloalkyl groups are optionally substituted with one to four R₇.

In another embodiment, each R₄ is independently selected from halogen, —OH, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl ring comprising 1 to 3 heteroatoms selected from O, N, and S, wherein the aryl, heteroaryl, cycloalkyl, and heterocycloalkyl groups are optionally substituted with one to four R₇.

In another embodiment, each R₄ is independently selected from —C(O)OR₆, —C(O)NR₆R₆′, and —NR₆C(O)R₆′. In another embodiment, each R₄ is independently selected from —C(O)OR₆, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to four R₇. In yet another embodiment, each R₄ is independently selected from (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to four R₇. In another embodiment, each R₄ is independently selected from (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In another embodiment, each R₄ is independently selected from (C₆-C₁₀)aryl and 5- or 6-membered heteroaryl comprising 1 to 3 heteroatoms selected from O, N, and S, wherein the aryl and heteroaryl are optionally substituted with one to three R₇. In yet another embodiment, each R₄ is independently selected from (C₆-C₁₀)aryl and 5- or 6-membered heteroaryl comprising 1 to 3 heteroatoms selected from O, N, and S, wherein the aryl and heteroaryl are substituted with one to three R₇.

In another embodiment, each R₄ is independently selected from (C₃-C₈)cycloalkyl and 5- to 7-membered heterocycloalkyl comprising 1 to 3 heteroatoms selected from O, N, and S, wherein the cycloalkyl and heterocycloalkyl groups are optionally substituted with one to three R₇. In another embodiment, each R₄ is independently selected from (C₃-C₈)cycloalkyl and 5- to 7-membered heterocycloalkyl comprising 1 to 3 heteroatoms selected from O, N, and S, wherein the cycloalkyl and heterocycloalkyl groups are substituted with one to three R₇.

In another embodiment, each R₄ is independently (C₆-C₁₀)aryl optionally substituted with one to three R₇. In yet another embodiment, each R₄ is independently 5- or 6-membered heteroaryl comprising 1 to 3 heteroatoms selected from O, N, and S, optionally substituted with one to three R₇.

In another embodiment, each R₄ is (C₃-C₈)cycloalkyl optionally substituted with one to three R₇. In another embodiment, each R₄ is independently 5- to 7-membered heterocycloalkyl comprising 1 to 3 heteroatoms selected from O, N, and S, optionally substituted with one to three R₇.

In some embodiments of the formulae above, each R₅ is independently selected from (C₁-C₆)alkyl, (C₂-C₆)alkenyl, (C₂-C₆)alkynyl, (C₁-C₆)alkoxy, (C₁-C₆)haloalkyl, (C₁-C₆)haloalkoxy, (C₁-C₆)hydroxyalkyl, halogen, —OH, —NH₂, CN, (C₃-C₇)cycloalkyl, 5- to 7-membered heterocycloalkyl comprising 1 to 3 heteroatoms selected from O, N, and S, (C₆-C₁₀)aryl, and 5- or 6-membered heteroaryl comprising 1 to 3 heteroatoms selected from O, N, and S. In another embodiment, each R₅ is independently selected from (C₁-C₆)alkyl, (C₂-C₆)alkenyl, (C₂-C₆)alkynyl, (C₁-C₆)alkoxy, (C₁-C₆)haloalkyl, (C₁-C₆)haloalkoxy, (C₁-C₆)hydroxyalkyl, halogen, —OH, —NH₂, and CN. In yet another embodiment, each R₅ is independently selected from (C₃-C₇)cycloalkyl, 5- to 7-membered heterocycloalkyl comprising 1 to 3 heteroatoms selected from O, N, and S, (C₆-C₁₀)aryl, and 5- or 6-membered heteroaryl comprising 1 to 3 heteroatoms selected from O, N, and S.

In another embodiment, each R₅ is independently selected from (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —NH₂, CN, (C₃-C₇)cycloalkyl, 5- to 7-membered heterocycloalkyl comprising 1 to 3 heteroatoms selected from O, N, and S, (C₆-C₁₀)aryl, and 5- or 6-membered heteroaryl comprising 1 to 3 heteroatoms selected from O, N, and S.

In another embodiment, each R₅ is independently selected from (C₁-C₆)alkyl, (C₁-C₆)alkoxy, (C₁-C₆)haloalkyl, and (C₁-C₆)haloalkoxy. In yet another embodiment, each R₅ is independently selected from (C₁-C₆)hydroxyalkyl, halogen, —OH, —NH₂, and CN. In another embodiment, each R₅ is independently selected from (C₁-C₆)alkyl, (C₁-C₆)alkoxy, (C₁-C₆)haloalkyl, (C₁-C₆)haloalkoxy, (C₁-C₆)hydroxyalkyl, halogen, —OH, and CN.

In some embodiments of the formulae above, two R₅, when on adjacent atoms, together with the atoms to which they are attached form a (C₆-C₁₀)aryl ring or a 5- or 6-membered heteroaryl ring comprising 1 to 3 heteroatoms selected from O, N, and S, optionally substituted with one to four R₁₀. In another embodiment, two R₅, when on adjacent atoms, together with the atoms to which they are attached form a (C₆-C₁₀)aryl ring or a 5- or 6-membered heteroaryl ring comprising 1 to 3 heteroatoms selected from O, N, and S, optionally substituted with one to three R₁₀. In another embodiment, two R₅, when on adjacent atoms, together with the atoms to which they are attached form a (C₆-C₁₀)aryl ring or a 5-membered heteroaryl ring comprising 1 to 3 heteroatoms selected from O, N, and S, optionally substituted with one to three R₁₀. In another embodiment, two R₅, when on adjacent atoms, together with the atoms to which they are attached form a (C₆-C₁₀)aryl ring or a 6-membered heteroaryl ring comprising 1 to 3 heteroatoms selected from O, N, and S, optionally substituted with one to three R₁₀.

In another embodiment, two R₅, when on adjacent atoms, together with the atoms to which they are attached form a phenyl ring or a 5- or 6-membered heteroaryl ring comprising 1 to 3 heteroatoms selected from O, N, and S, optionally substituted with one to three R₁₀. In yet another embodiment, two R₅, when on adjacent atoms, together with the atoms to which they are attached form a phenyl ring or a 5-membered heteroaryl ring comprising 1 to 3 heteroatoms selected from O, N, and S, optionally substituted with one to three R₁₀. In another embodiment, two R₅, when on adjacent atoms, together with the atoms to which they are attached form a phenyl ring or a 6-membered heteroaryl ring comprising 1 to 3 heteroatoms selected from O, N, and S, optionally substituted with one to three R₁₀.

In another embodiment, two R₅, when on adjacent atoms, together with the atoms to which they are attached form a (C₆-C₁₀)aryl ring optionally substituted with one to three R₁₀. In yet another embodiment, two R₅, when on adjacent atoms, together with the atoms to which they are attached form a phenyl ring optionally substituted with one to three R₁₀. In another embodiment, two R₅, when on adjacent atoms, together with the atoms to which they are attached form a 5- or 6-membered heteroaryl ring comprising 1 to 3 heteroatoms selected from O, N, and S, optionally substituted with one to three R₁₀. In yet another embodiment, two R₅, when on adjacent atoms, together with the atoms to which they are attached form a 5-membered heteroaryl ring comprising 1 to 3 heteroatoms selected from O, N, and S, optionally substituted with one to three R₁₀. In another embodiment, two R₅, when on adjacent atoms, together with the atoms to which they are attached form a 6-membered heteroaryl ring comprising 1 to 3 heteroatoms selected from O, N, and S, optionally substituted with one to three R₁₀.

In some embodiments of the formulae above, two R₅ together with the atoms to which they are attached form a (C₃-C₇)cycloalkyl ring or a 4- to 7-membered heterocycloalkyl ring comprising 1 to 3 heteroatoms selected from O, N, and S optionally substituted with one to four R₁₀. In another embodiment, two R₅ together with the atoms to which they are attached form a (C₃-C₇)cycloalkyl ring or a 4- to 7-membered heterocycloalkyl ring comprising 1 to 3 heteroatoms selected from O, N, and S optionally substituted with one to three R₁₀. In yet another embodiment, two R₅ together with the atoms to which they are attached form a (C₃-C₇)cycloalkyl ring or a 5- to 7-membered heterocycloalkyl ring comprising 1 to 3 heteroatoms selected from O, N, and S optionally substituted with one to three R₁₀. In another embodiment, two R₅ together with the atoms to which they are attached form a (C₃-C₇)cycloalkyl ring or a 6- or 7-membered heterocycloalkyl ring comprising 1 to 3 heteroatoms selected from O, N, and S optionally substituted with one to three R₁₀.

In another embodiment, two R₅ together with the atoms to which they are attached form a (C₃-C₇)cycloalkyl ring or a 5- or 6-membered heterocycloalkyl ring comprising 1 to 3 heteroatoms selected from O, N, and S optionally substituted with one to three R₁₀. In yet another embodiment, two R₅ together with the atoms to which they are attached form a (C₃-C₇)cycloalkyl ring or a 4- to 6-membered heterocycloalkyl ring comprising 1 to 3 heteroatoms selected from O, N, and S optionally substituted with one to three R₁₀. In another embodiment, two R₅ together with the atoms to which they are attached form a (C₃-C₇)cycloalkyl ring or a 4- or 5-membered heterocycloalkyl ring comprising 1 to 3 heteroatoms selected from O, N, and S optionally substituted with one to three R₁₀. In yet another embodiment, two R₅ together with the atoms to which they are attached form a (C₃-C₇)cycloalkyl ring or a 5- to 7-membered heterocycloalkyl ring comprising 1 to 3 heteroatoms selected from O, N, and S optionally substituted with one three R₁₀.

In another embodiment, two R₅ together with the atoms to which they are attached form a (C₄-C₇)cycloalkyl ring or a 5- to 7-membered heterocycloalkyl ring comprising 1 to 3 heteroatoms selected from O, N, and S optionally substituted with one three R₁₀.

In another embodiment, two R₅ together with the atoms to which they are attached form a (C₅-C₇)cycloalkyl ring or a 5- to 7-membered heterocycloalkyl ring comprising 1 to 3 heteroatoms selected from O, N, and S optionally substituted with one three R₁₀. In another embodiment, two R₅ together with the atoms to which they are attached form a (C₆-C₇)cycloalkyl ring or a 5- to 7-membered heterocycloalkyl ring comprising 1 to 3 heteroatoms selected from O, N, and S optionally substituted with one three R₁₀. In another embodiment, two R₅ together with the atoms to which they are attached form a (C₅-C₆)cycloalkyl ring or a 5- to 7-membered heterocycloalkyl ring comprising 1 to 3 heteroatoms selected from O, N, and S optionally substituted with one three R₁₀. In another embodiment, two R₅ together with the atoms to which they are attached form a (C₄-C₆)cycloalkyl ring or a 5- to 7-membered heterocycloalkyl ring comprising 1 to 3 heteroatoms selected from O, N, and S optionally substituted with one three R₁₀. In another embodiment, two R₅ together with the atoms to which they are attached form a (C₃-C₆)cycloalkyl ring or a 5- to 7-membered heterocycloalkyl ring comprising 1 to 3 heteroatoms selected from O, N, and S optionally substituted with one three R₁₀. In another embodiment, two R₅ together with the atoms to which they are attached form a (C₃-C₅)cycloalkyl ring or a 5- to 7-membered heterocycloalkyl ring comprising 1 to 3 heteroatoms selected from O, N, and S optionally substituted with one three R₁₀. In another embodiment, two R₅ together with the atoms to which they are attached form a (C₃-C₄)cycloalkyl ring or a 5- to 7-membered heterocycloalkyl ring comprising 1 to 3 heteroatoms selected from O, N, and S optionally substituted with one three R₁₀.

In another embodiment, two R₅, together with the atoms to which they are attached form a (C₃-C₇)cycloalkyl ring optionally substituted with one three R₁₀. In another embodiment, two R₅, together with the atoms to which they are attached form a (C₄-C₇)cycloalkyl ring optionally substituted with one three R₁₀. In another embodiment, two R₅, together with the atoms to which they are attached form a (C₅-C₇)cycloalkyl ring optionally substituted with one three R₁₀. In yet another embodiment, two R₅, together with the atoms to which they are attached form a (C₆-C₇)cycloalkyl ring optionally substituted with one three R₁₀. In another embodiment, two R₅, together with the atoms to which they are attached form a (C₃-C₆)cycloalkyl ring optionally substituted with one three R₁₀. In yet another embodiment, two R₅, together with the atoms to which they are attached form a (C₃-C₅)cycloalkyl ring optionally substituted with one three R₁₀. In another embodiment, two R₅, together with the atoms to which they are attached form a (C₃-C₄)cycloalkyl ring optionally substituted with one three R₁₀.

In another embodiment, two R₅ together with the atoms to which they are attached form a 4- to 7-membered heterocycloalkyl ring comprising 1 to 3 heteroatoms selected from O, N, and S optionally substituted with one three R₁₀. In yet another embodiment, two R₅ together with the atoms to which they are attached form a 5- to 7-membered heterocycloalkyl ring comprising 1 to 3 heteroatoms selected from O, N, and S optionally substituted with one three R₁₀. In another embodiment, two R₅ together with the atoms to which they are attached form a 6- or 7-membered heterocycloalkyl ring comprising 1 to 3 heteroatoms selected from O, N, and S optionally substituted with one three R₁₀. In yet another embodiment, two R₅ together with the atoms to which they are attached form a 5- or 6-membered heterocycloalkyl ring comprising 1 to 3 heteroatoms selected from O, N, and S optionally substituted with one three R₁₀. In another embodiment, two R₅ together with the atoms to which they are attached form a 4- or 5-membered heterocycloalkyl ring comprising 1 to 3 heteroatoms selected from O, N, and S optionally substituted with one three R₁₀. In another embodiment, two R₅ together with the atoms to which they are attached form a 4- to 6-membered heterocycloalkyl ring comprising 1 to 3 heteroatoms selected from O, N, and S optionally substituted with one three R₁₀.

In some embodiments of the formulae above, R₆ is H or (C₁-C₃)alkyl. In another embodiment, R₆ is H or (C₆-C₁₀)aryl. In yet another embodiment, R₆ is (C₁-C₃)alkyl or (C₆-C₁₀)aryl. In another embodiment, R₆ is H, methyl, ethyl, n-propyl, or isopropyl. In another embodiment, R₆ is H, methyl or ethyl. In yet another embodiment, R₆ is H or methyl. In another embodiment, R₆ is H.

In some embodiments of the formulae above, R₆′ is H or (C₁-C₃)alkyl. In another embodiment, R₆′ is H or (C₆-C₁₀)aryl. In yet another embodiment, R₆′ is (C₁-C₃)alkyl or (C₆-C₁₀)aryl. In another embodiment, R₆′ is H, methyl, ethyl, n-propyl, or isopropyl. In another embodiment, R₆′ is H, methyl or ethyl. In yet another embodiment, R₆′ is H or methyl. In another embodiment, R₆′ is H.

In some embodiments of the formulae above, each R₇ is independently selected from (C₁-C₆)alkyl, (C₂-C₆)alkenyl, (C₂-C₆)alkynyl, (C₁-C₆)alkoxy, (C₁-C₆)haloalkyl, (C₁-C₆)haloalkoxy, —C(O)R₈, —(CH₂)₀₋₃C(O)OR₈, —C(O)NR₈R₉, —NR₈C(O)R₉, —NR₈C(O)OR₉, —S(O)ₚNR₈R₉, —S(O)ₚR₁₂, (C₁-C₆)hydroxyalkyl, halogen, —OH, —O(CH₂)₁₋₃CN, —NH₂, CN, —O(CH₂)₀₋₃(C₆-C₁₀)aryl, adamantyl, —O(CH₂)₀₋₃-5- or 6-membered heteroaryl comprising 1 to 3 heteroatoms selected from O, N, and S, (C₆-C₁₀)aryl, monocyclic or bicyclic 5- to 10-membered heteroaryl comprising 1 to 3 heteroatoms selected from O, N, and S, (C₃-C₇)cycloalkyl, and 5- to 7-membered heterocycloalkyl comprising 1 to 3 heteroatoms selected from O, N, and S, wherein the alkyl is optionally substituted with one to four Rn, and the aryl, heteroaryl, and heterocycloalkyl are optionally substituted with one to four substituent each independently selected from halogen, (C₁-C₆)alkyl, (C₁-C₆)haloalkyl, and (C₁-C₆)alkoxy. In another embodiment, each R₇ is independently selected from (C₁-C₆)alkyl, (C₂-C₆)alkenyl, (C₂-C₆)alkynyl, (C₁-C₆)alkoxy, (C₁-C₆)haloalkyl, (C₁-C₆)haloalkoxy, —C(O)R₈, —(CH₂)₀₋₃C(O)OR₈, —C(O)NR₈R₉, —NR₈C(O)R₉, —NR₈C(O)OR₉, —S(O)ₚNR₈R₉, —S(O)ₚR₁₂, (C₁-C₆)hydroxyalkyl, halogen, —OH, —O(CH₂)₁₋₃CN, —NH₂, CN, —O(CH₂)₀₋₃(C₆-C₁₀)aryl, —O(CH₂)₀₋₃-5- or 6-membered heteroaryl comprising 1 to 3 heteroatoms selected from O, N, and S, (C₆-C₁₀)aryl, monocyclic or bicyclic 5- to 10-membered heteroaryl comprising 1 to 3 heteroatoms selected from O, N, and S, (C₃-C₇)cycloalkyl, and 5- to 7-membered heterocycloalkyl comprising 1 to 3 heteroatoms selected from O, N, and S, wherein the alkyl is optionally substituted with one to four Ru, and the aryl, heteroaryl, and heterocycloalkyl are optionally substituted with one to four substituent each independently selected from halogen, (C₁-C₆)alkyl, (C₁-C₆)haloalkyl, and (C₁-C₆)alkoxy.

In another embodiment, each R₇ is independently selected from (C₁-C₆)alkyl, (C₁-C₆)alkoxy, (C₁-C₆)haloalkyl, (C₁-C₆)haloalkoxy, —C(O)R₈, —(CH₂)₀₋₃C(O)OR₈, —C(O)NR₈R₉, —NR₈C(O)R₉, —NR₈C(O)OR₉, —S(O)ₚNR₈R₉, —S(O)ₚR₁₂, (C₁-C₆)hydroxyalkyl, halogen, —OH, —O(CH₂)₁₋₃CN, —NH₂, CN, —O(CH₂)₀₋₃(C₆-C₁₀)aryl, —O(CH₂)₀₋₃-5- or 6-membered heteroaryl comprising 1 to 3 heteroatoms selected from O, N, and S, (C₆-C₁₀)aryl, monocyclic or bicyclic 5- to 10-membered heteroaryl comprising 1 to 3 heteroatoms selected from O, N, and S, (C₃-C₇)cycloalkyl, and 5- to 7-membered heterocycloalkyl comprising 1 to 3 heteroatoms selected from O, N, and S, wherein the alkyl is optionally substituted with one to four Rn, and the aryl, heteroaryl, and heterocycloalkyl are optionally substituted with one to four substituent each independently selected from halogen, (C₁-C₆)alkyl, (C₁-C₆)haloalkyl, and (C₁-C₆)alkoxy.

In another embodiment, each R₇ is independently selected from —(CH₂)₀₋₃C(O)OR₈, —NR₈C(O)OR₉, —S(O)ₚNR₈R₉, —S(O)ₚR₁₂, (C₁-C₆)hydroxyalkyl, halogen, —OH, —O(CH₂)₁₋₃CN, —NH₂, CN, —O(CH₂)₀₋₃(C₆-C₁₀)aryl, —O(CH₂)₀₋₃-5- or 6-membered heteroaryl comprising 1 to 3 heteroatoms selected from O, N, and S, bicyclic 9- or 10-membered heteroaryl comprising 1 to 3 heteroatoms selected from O, N, and S, wherein the aryl and heteroaryl and heterocycloalkyl are optionally substituted with one or more substituent each independently selected from halogen, (C₁-C₆)alkyl, (C₁-C₆)haloalkyl, and (C₁-C₆)alkoxy.

In another embodiment, each R₇ is independently selected from (C₁-C₆)alkyl, (C₂-C₆)alkenyl, (C₂-C₆)alkynyl, (C₁-C₆)alkoxy, (C₁-C₆)haloalkyl, (C₁-C₆)haloalkoxy, —C(O)R₈, —C(O)OR₈, —C(O)NR₈R₉, —NR₈C(O)R₉, (C₁-C₆)hydroxyalkyl, halogen, —OH, —NH₂, CN, (C₆-C₁₀)aryl, 5- or 6-membered heteroaryl comprising 1 to 3 heteroatoms selected from O, N, and S, (C₃-C₇)cycloalkyl, and 5- to 7-membered heterocycloalkyl comprising 1 to 3 heteroatoms selected from O, N, and S. In another embodiment, each R₇ is independently selected from (C₁-C₆)alkyl, (C₂-C₆)alkenyl, (C₂-C₆)alkynyl, (C₁-C₆)alkoxy, (C₁-C₆)haloalkyl, (C₁-C₆)haloalkoxy, —C(O)R₈, —C(O)OR₈, —C(O)NR₈R₉, —NR₈C(O)R₉, (C₁-C₆)hydroxyalkyl, halogen, —OH, —NH₂, and CN.

In another embodiment, each R₇ is independently selected from (C₁-C₆)alkyl, (C₁-C₆)alkoxy, (C₁-C₆)haloalkyl, (C₁-C₆)haloalkoxy, —C(O)R₈, —C(O)OR₈, —C(O)NR₈R₉, —NR₈C(O)R₉, (C₁-C₆)hydroxyalkyl, halogen, —OH, —NH₂, and CN. In yet another embodiment, each R₇ is independently selected from (C₁-C₆)alkyl, (C₁-C₆)alkoxy, (C₁-C₆)haloalkyl, (C₁-C₆)haloalkoxy. In another embodiment, each R₇ is independently selected from —C(O)R₈, —C(O)OR₈, —C(O)NR₈R₉, —NR₈C(O)R₉, (C₁-C₆)hydroxyalkyl, halogen, —OH, —NH₂, and CN. In another embodiment, each R₇ is independently selected from (C₆-C₁₀)aryl, 5- or 6-membered heteroaryl comprising 1 to 3 heteroatoms selected from O, N, and S, (C₃-C₇)cycloalkyl, and 5- to 7-membered heterocycloalkyl comprising 1 to 3 heteroatoms selected from O, N, and S.

In another embodiment, each R₇ is independently selected from (C₁-C₆)alkyl, (C₁-C₆)alkoxy, (C₁-C₆)haloalkyl, (C₁-C₆)haloalkoxy, —C(O)R₈, —C(O)OR₈, —C(O)NR₈R₉, —NR₈C(O)R₉, (C₁-C₆)hydroxyalkyl, halogen, —OH, —NH₂, CN, (C₆-C₁₀)aryl, 5- or 6-membered heteroaryl comprising 1 to 3 heteroatoms selected from O, N, and S, (C₃-C₇)cycloalkyl, and 5- to 7-membered heterocycloalkyl comprising 1 to 3 heteroatoms selected from O, N, and S. In yet another embodiment, each R₇ is independently selected from (C₁-C₆)alkyl, (C₁-C₆)alkoxy, halogen, —OH, CN, and (C₆-C₁₀)aryl.

In some embodiments of the formulae above, two R₇, when on adjacent atoms, together with the atoms to which they are attached form a (C₆-C₁₀)aryl ring or a 5- or 6-membered heteroaryl ring comprising 1 to 3 heteroatoms selected from O, N, and S, optionally substituted with one or more R₁₀. In another embodiment, two R₇, when on adjacent atoms, together with the atoms to which they are attached form a (C₆-C₁₀)aryl ring optionally substituted with one or more R₁₀. In another embodiment, two R₇, when on adjacent atoms, together with the atoms to which they are attached form a 5- or 6-membered heteroaryl ring comprising 1 to 3 heteroatoms selected from O, N, and S, optionally substituted with one or more R₁₀. In another embodiment, two R₇ together with the atoms to which they are attached form a (C₅-C₇) cycloalkyl ring optionally substituted with one or more R₁₀. In another embodiment, two R₇ together with the atoms to which they are attached form a 5- to 7-membered heterocycloalkyl ring comprising 1 to 3 heteroatoms selected from O, N, and S, optionally substituted with one or more R₁₀.

In another embodiment, two R₇, when on adjacent atoms, together with the atoms to which they are attached form a (C₆-C₁₀)aryl ring or a 5- or 6-membered heteroaryl ring comprising 1 to 3 heteroatoms selected from O, N, and S, optionally substituted with one to four R₁₀, or two R₇, when on adjacent atoms, together with the atoms to which they are attached form a (C₅-C₇)cycloalkyl ring or a 5- to 7-membered heterocycloalkyl ring comprising 1 to 3 heteroatoms selected from O, N, and S, optionally substituted with one to four R₁₀.

In another embodiment, two R₇, when on adjacent atoms, together with the atoms to which they are attached form a (C₅-C₇)cycloalkyl ring or a 5- to 7-membered heterocycloalkyl ring comprising 1 to 3 heteroatoms selected from O, N, and S, optionally substituted with one to four R₁₀. In another embodiment, two R₇, when on adjacent atoms, together with the atoms to which they are attached form a (C₅-C₇)cycloalkyl ring optionally substituted with one to four R₁₀. In another embodiment, two R₇, when on adjacent atoms, together with the atoms to which they are attached form a 5- to 7-membered heterocycloalkyl ring comprising 1 to 3 heteroatoms selected from O, N, and S, optionally substituted with one to four R₁₀.

In some embodiments of the formulae above, R₈ is H or (C₁-C₃)alkyl. In another embodiment, R₈ is H, methyl, ethyl, n-propyl, or isopropyl. In another embodiment, R₈ is H, methyl or ethyl. In yet another embodiment, R₈ is H or methyl. In another embodiment, R₈ is H In some embodiments of the formulae above, R₉ is H or (C₁-C₃)alkyl. In another embodiment, R₉ is H, methyl, ethyl, n-propyl, or isopropyl. In another embodiment, R₉ is H, methyl or ethyl. In yet another embodiment, R₉ is H or methyl. In another embodiment, R₉ is H.

In some embodiments of the formulae above, each R₁₀ is independently selected from (C₁-C₆)alkyl, (C₁-C₆)alkoxy, (C₁-C₆)haloalkyl, (C₁-C₆)haloalkoxy, (C₁-C₆)hydroxyalkyl, and halogen. In another embodiment, each R₁₀ is independently selected from —OH, —NH₂, and CN. In yet another embodiment, each R₁₀ is independently selected from (C₁-C₆)alkyl, (C₁-C₆)alkoxy, (C₁-C₆)haloalkyl, (C₁-C₆)haloalkoxy, and halogen. In another embodiment, each R₁₀ is independently selected from (C₁-C₆)alkyl, (C₁-C₆)haloalkyl, and halogen. In yet another embodiment, each R₁₀ is independently selected from (C₁-C₆)alkyl and halogen.

In some embodiments of the formulae above, two R₁₀ together with the carbon atom to which they are attached form a ═(O).

In some embodiments of the formulae above, each R₁₁ is independently selected from CN, (C₁-C₆)alkoxy, (C₆-C₁₀)aryl, and 5- to 7-membered heterocycloalkyl comprising 1 to 3 heteroatoms selected from O, N, and S, wherein the aryl and heterocycloalkyl are optionally substituted with one to four substituents each independently selected from (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —NH₂, and CN. In another embodiment, each R₁₁ is independently selected from CN, (C₁-C₆)alkoxy, (C₆-C₁₀)aryl, and 5- to 7-membered heterocycloalkyl comprising 1 to 3 heteroatoms selected from O, N, and S, wherein the aryl and heterocycloalkyl are optionally substituted with one to three substituents each independently selected from (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —NH₂, and CN. In yet another embodiment, each R₁₁ is independently selected from CN, (C₁-C₆)alkoxy, and (C₆-C₁₀)aryl, wherein the aryl is optionally substituted with one to three substituents each independently selected from (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —NH₂, and CN.

In another embodiment, each R₁₁ is independently selected from CN, (C₁-C₆)alkoxy, and 5- to 7-membered heterocycloalkyl comprising 1 to 3 heteroatoms selected from O, N, and S, wherein the heterocycloalkyl is optionally substituted with one to four substituents each independently selected from (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —NH₂, and CN. In another embodiment, each R₁₁ is independently selected from CN and (C₁-C₆)alkoxy. In yet another embodiment, each R₁₁ is independently selected from (C₆-C₁₀)aryl and 5- to 7-membered heterocycloalkyl comprising 1 to 3 heteroatoms selected from O, N, and S, wherein the aryl and heterocycloalkyl are optionally substituted with one to four substituents each independently selected from (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —NH₂, and CN.

In some embodiments of the formulae above, R₁₂ is (C₁-C₆)alkyl, (C₁-C₆)haloalkyl, (C₆-C₁₀)aryl, or 5- or 6-membered heterocycloalkyl comprising 1 to 3 heteroatoms selected from O, N, and S. In another embodiment, R₁₂ is (C₁-C₆)alkyl, (C₁-C₆)haloalkyl, phenyl, or 5- or 6-membered heterocycloalkyl comprising 1 to 3 heteroatoms selected from O, N, and S. In another embodiment, R₁₂ is (C₁-C₄)alkyl, (C₁-C₄)haloalkyl, phenyl, or 5- or 6-membered heterocycloalkyl comprising 1 to 3 heteroatoms selected from O, N, and S.

In some embodiments of the formulae above, n is 0, 1, or 2. In another embodiment, n is 1, 2, or 3. In another embodiment, n is 0 or 1. In yet another embodiment, n is 1 or 2. In another embodiment, n is 2 or 3. In yet another embodiment, n is 0. In yet another embodiment, n is 1. In another embodiment, n is 2. In yet another embodiment, n is 3.

In some embodiments of the formulae above, Rₓ is H and n is 0. In another embodiment, Rₓ is H and n is 1. In another embodiment, Rₓ is H and n is 2.

In some embodiments of the formulae above, Rₓ is H, n is 0, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN. In another embodiment, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, and R₃ is (C₁-C₆)alkyl optionally substituted with one to three R₄. In another embodiment, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, and R₃ is (C₁-C₆)alkyl substituted with one to three R₄.

In some embodiments of the formulae above, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, R₃ is (C₁-C₆)alkyl optionally substituted with one to three R₄, and each R₄ is independently selected from —C(O)OR₆, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇. In another embodiment, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, R₃ is (C₁-C₆)alkyl substituted with one to three R₄, and each R₄ is independently selected from —C(O)OR₆, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In some embodiments of the formulae above, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, R₃ is (C₁-C₆)alkyl optionally substituted with one to three R₄, and each R₄ is independently selected from (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇. In another embodiment, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, R₃ is (C₁-C₆)alkyl substituted with one to three R₄, and each R₄ is independently selected from (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In some embodiments of the formulae above, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, R₃ is (C₁-C₆)alkyl optionally substituted with one to three R₄, and each R₄ is independently selected from halogen, —OH, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇. In another embodiment, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, R₃ is (C₁-C₆)alkyl substituted with one to three R₄, and each R₄ is independently selected from halogen, —OH, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In some embodiments of the formulae above, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, R₃ is (C₁-C₆)alkyl optionally substituted with one to three R₄, and each R₄ is independently selected from halogen, —OH, phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇. In another embodiment, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, R₃ is (C₁-C₆)alkyl substituted with one to three R₄, and each R₄ is independently selected from halogen, —OH, phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇.

In some embodiments of the formulae above, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, R₃ is (C₁-C₆)alkyl optionally substituted with one to three R₄, and each R₄ is independently selected from phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇. In another embodiment, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, R₃ is (C₁-C₆)alkyl substituted with one to three R₄, and each R₄ is independently selected from phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇.

In some embodiments of the formulae above, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, R₃ is (C₁-C₆)alkyl optionally substituted with one to three R₄, and each R₄ is independently selected from phenyl and 5- or 6-membered heteroaryl comprising 1 to 3 heteroatoms selected from O, N, and S, wherein the phenyl and heteroaryl groups are optionally substituted with one to three R₇. In another embodiment, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, R₃ is (C₁-C₆)alkyl substituted with one to three R₄, and each R₄ is independently selected from phenyl and 5- or 6-membered heteroaryl comprising 1 to 3 heteroatoms selected from O, N, and S, wherein the phenyl and heteroaryl groups are optionally substituted with one to three R₇.

In some embodiments of the formulae above, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, R₃ is (C₁-C₆)alkyl optionally substituted with one to three R₄, and each R₄ is phenyl optionally substituted with one to three R₇. In another embodiment, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, R₃ is (C₁-C₆)alkyl substituted with one to three R₄, and each R₄ is phenyl optionally substituted with one to three R₇.

In some embodiments of the formulae above, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, R₃ is (C₁-C₆)alkyl optionally substituted with one to three R₄, and each R₄ is 5- or 6-membered heteroaryl comprising 1 to 3 heteroatoms selected from O, N, and S, optionally substituted with one to three R₇. In another embodiment, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, R₃ is (C₁-C₆)alkyl substituted with one to three R₄, and each R₄ is 5- or 6-membered heteroaryl comprising 1 to 3 heteroatoms selected from O, N, and S, optionally substituted with one to three R₇.

In some embodiments of the formulae above, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, R₃ is (C₁-C₆)alkyl optionally substituted with one to three R₄, and each R₄ is (C₃-C₈)cycloalkyl optionally substituted with one to three R₇. In another embodiment, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, R₃ is (C₁-C₆)alkyl substituted with one to three R₄, and each R₄ is (C₃-C₈)cycloalkyl optionally substituted with one to three R₇.

In some embodiments of the formulae above, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, R₃ is (C₁-C₆)alkyl optionally substituted with one to three R₄, and each R₄ is 5- to 7-membered heterocycloalkyl comprising 1 to 3 heteroatoms selected from O, N, and S, optionally substituted with one to three R₇.

In another embodiment, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, R₃ is (C₁-C₆)alkyl substituted with one to three R₄, and each R₄ is 5- to 7-membered heterocycloalkyl comprising 1 to 3 heteroatoms selected from O, N, and S, optionally substituted with one to three R₇.

In some embodiments of the formulae above, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, and R₃ is (C₆-C₁₀)aryl, (C₃-C₈)cycloalkyl, or 5- to 7-membered heterocycloalkyl comprising 1 to 3 heteroatoms selected from O, N, and S, wherein the aryl, cycloalkyl, and heterocycloalkyl are optionally substituted with one to three R₅.

In some embodiments of the formulae above, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, and R₃ is (C₆-C₁₀)aryl, (C₃-C₈)cycloalkyl, or 5- to 7-membered heterocycloalkyl comprising 1 to 3 heteroatoms selected from O, N, and S.

In some embodiments of the formulae above, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, and R₃ is (C₆-C₁₀)aryl, 5- or 6-membered heteroaryl comprising 1 to 3 heteroatoms selected from O, N, and S, or (C₃-C₈)cycloalkyl, wherein the aryl, heteroaryl and cycloalkyl are optionally substituted with one to three R₅.

In some embodiments of the formulae above, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, and R₃ is (C₆-C₁₀)aryl, 5- or 6-membered heteroaryl comprising 1 to 3 heteroatoms selected from O, N, and S, or (C₃-C₈)cycloalkyl.

In some embodiments of the formulae above, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, and R₃ is (C₆-C₁₀)aryl optionally substituted with one to three R₅.

In some embodiments of the formulae above, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, and R₃ is 5- or 6-membered heteroaryl comprising 1 to 3 heteroatoms selected from O, N, and S optionally substituted with one to three R₅.

In some embodiments of the formulae above, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂ NH₂, —(CH₂)₀₋₂NH(C₁-C₆)alkyl, —(CH₂)₀₋₂N((C₁-C₆)alkyl)₂, —C(O)NH₂, —C(O)OH or CN, and R₃ is (C₃-C₈)cycloalkyl optionally substituted with one to three R₅.

In some embodiments of the formulae above, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, and R₃ is 5- to 7-membered heterocycloalkyl comprising 1 to 3 heteroatoms selected from O, N, and S, optionally substituted with one to three R₅.

In some embodiments of the formulae above, Rₓ is H, n is 1, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN. In another embodiment, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, and R₃ is (C₁-C₆)alkyl optionally substituted with one to three R₄. In another embodiment, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, and R₃ is (C₁-C₆)alkyl substituted with one to three R₄.

In some embodiments of the formulae above, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, R₃ is (C₁-C₆)alkyl optionally substituted with one to three R₄, and each R₄ is independently selected from —C(O)OR₆, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇. In another embodiment, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, R₃ is (C₁-C₆)alkyl substituted with one to three R₄, and each R₄ is independently selected from —C(O)OR₆, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In some embodiments of the formulae above, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂ NH₂, —(CH₂)₀₋₂NH(C₁-C₆)alkyl, —(CH₂)₀₋₂N((C₁-C₆)alkyl)₂, —C(O)NH₂, —C(O)OH or CN, R₃ is (C₁-C₆)alkyl optionally substituted with one to three R₄, and each R₄ is independently selected from (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇. In another embodiment, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, R₃ is (C₁-C₆)alkyl substituted with one to three R₄, and each R₄ is independently selected from (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In some embodiments of the formulae above, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, R₃ is (C₁-C₆)alkyl optionally substituted with one to three R₄, and each R₄ is independently selected from halogen, —OH, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇. In another embodiment, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, R₃ is (C₁-C₆)alkyl substituted with one to three R₄, and each R₄ is independently selected from halogen, —OH, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In some embodiments of the formulae above, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, R₃ is (C₁-C₆)alkyl optionally substituted with one to three R₄, and each R₄ is independently selected from halogen, —OH, phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇. In another embodiment, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, R₃ is (C₁-C₆)alkyl substituted with one to three R₄, and each R₄ is independently selected from halogen, —OH, phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇.

In some embodiments of the formulae above, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, R₃ is (C₁-C₆)alkyl optionally substituted with one to three R₄, and each R₄ is independently selected from phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇. In another embodiment, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, R₃ is (C₁-C₆)alkyl substituted with one to three R₄, and each R₄ is independently selected from phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇.

In some embodiments of the formulae above, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, R₃ is (C₁-C₆)alkyl optionally substituted with one to three R₄, and each R₄ is independently selected from phenyl and 5- or 6-membered heteroaryl comprising 1 to 3 heteroatoms selected from O, N, and S, wherein the phenyl and heteroaryl groups are optionally substituted with one to three R₇. In another embodiment, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, R₃ is (C₁-C₆)alkyl substituted with one to three R₄, and each R₄ is independently selected from phenyl and 5- or 6-membered heteroaryl comprising 1 to 3 heteroatoms selected from O, N, and S, wherein the phenyl and heteroaryl groups are optionally substituted with one to three R₇.

In some embodiments of the formulae above, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, R₃ is (C₁-C₆)alkyl optionally substituted with one to three R₄, and each R₄ is phenyl optionally substituted with one to three R₇. In another embodiment, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, R₃ is (C₁-C₆)alkyl substituted with one to three R₄, and each R₄ is phenyl optionally substituted with one to three R₇.

In some embodiments of the formulae above, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, R₃ is (C₁-C₆)alkyl optionally substituted with one to three R₄, and each R₄ is 5- or 6-membered heteroaryl comprising 1 to 3 heteroatoms selected from O, N, and S, optionally substituted with one to three R₇. In another embodiment, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, R₃ is (C₁-C₆)alkyl substituted with one to three R₄, and each R₄ is 5- or 6-membered heteroaryl comprising 1 to 3 heteroatoms selected from O, N, and S, optionally substituted with one to three R₇.

In some embodiments of the formulae above, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, R₃ is (C₁-C₆)alkyl optionally substituted with one to three R₄, and each R₄ is (C₃-C₈)cycloalkyl optionally substituted with one to three R₇.

In another embodiment, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, R₃ is (C₁-C₆)alkyl substituted with one to three R₄, and each R₄ is (C₃-C₈)cycloalkyl optionally substituted with one to three R₇.

In some embodiments of the formulae above, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, R₃ is (C₁-C₆)alkyl optionally substituted with one to three R₄, and each R₄ is 5- to 7-membered heterocycloalkyl comprising 1 to 3 heteroatoms selected from O, N, and S, optionally substituted with one to three R₇.

In another embodiment, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, R₃ is (C₁-C₆)alkyl substituted with one to three R₄, and each R₄ is 5- to 7-membered heterocycloalkyl comprising 1 to 3 heteroatoms selected from O, N, and S, optionally substituted with one to three R₇.

In some embodiments of the formulae above, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, and R₃ is (C₆-C₁₀)aryl, (C₃-C₈)cycloalkyl, or 5- to 7-membered heterocycloalkyl comprising 1 to 3 heteroatoms selected from O, N, and S, wherein the aryl, cycloalkyl, and heterocycloalkyl are optionally substituted with one to three R₅.

In some embodiments of the formulae above, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, and R₃ is (C₆-C₁₀)aryl, (C₃-C₈)cycloalkyl, or 5- to 7-membered heterocycloalkyl comprising 1 to 3 heteroatoms selected from O, N, and S.

In some embodiments of the formulae above, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, and R₃ is (C₆-C₁₀)aryl, 5- or 6-membered heteroaryl comprising 1 to 3 heteroatoms selected from O, N, and S, or (C₃-C₈)cycloalkyl, wherein the aryl, heteroaryl and cycloalkyl are optionally substituted with one to three R₅.

In some embodiments of the formulae above, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, and R₃ is (C₆-C₁₀)aryl, 5- or 6-membered heteroaryl comprising 1 to 3 heteroatoms selected from O, N, and S, or (C₃-C₈)cycloalkyl.

In some embodiments of the formulae above, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, and R₃ is (C₆-C₁₀)aryl optionally substituted with one to three R₅.

In some embodiments of the formulae above, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, and R₃ is 5- or 6-membered heteroaryl comprising 1 to 3 heteroatoms selected from O, N, and S optionally substituted with one to three R₅.

In some embodiments of the formulae above, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, and R₃ is (C₃-C₈)cycloalkyl optionally substituted with one to three R₅.

In some embodiments of the formulae above, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN, and R₃ is 5- to 7-membered heterocycloalkyl comprising 1 to 3 heteroatoms selected from O, N, and S, optionally substituted with one to three R₅.

In some embodiments of the formulae above, Rₓ is H, n is 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN.

In some embodiments of the formulae above, Rₓ is H, n is 0, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN. In another embodiment, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, and R₃ is (C₁-C₆)alkyl optionally substituted with one to three R₄. In another embodiment, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, and R₃ is (C₁-C₆)alkyl substituted with one to three R₄.

In some embodiments of the formulae above, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, R₃ is (C₁-C₆)alkyl optionally substituted with one to three R₄, and each R₄ is independently selected from —C(O)OR₆, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇. In another embodiment, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, R₃ is (C₁-C₆)alkyl substituted with one to three R₄, and each R₄ is independently selected from —C(O)OR₆, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In some embodiments of the formulae above, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, R₃ is (C₁-C₆)alkyl optionally substituted with one to three R₄, and each R₄ is independently selected from (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇. In another embodiment, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, R₃ is (C₁-C₆)alkyl substituted with one to three R₄, and each R₄ is independently selected from (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In some embodiments of the formulae above, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, R₃ is (C₁-C₆)alkyl optionally substituted with one to three R₄, and each R₄ is independently selected from halogen, —OH, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇. In another embodiment, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, R₃ is (C₁-C₆)alkyl substituted with one to three R₄, and each R₄ is independently selected from halogen, —OH, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In some embodiments of the formulae above, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, R₃ is (C₁-C₆)alkyl optionally substituted with one to three R₄, and each R₄ is independently selected from halogen, —OH, phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇. In another embodiment, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, R₃ is (C₁-C₆)alkyl substituted with one to three R₄, and each R₄ is independently selected from halogen, —OH, phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇.

In some embodiments of the formulae above, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, R₃ is (C₁-C₆)alkyl optionally substituted with one to three R₄, and each R₄ is independently selected from phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇. In another embodiment, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, R₃ is (C₁-C₆)alkyl substituted with one to three R₄, and each R₄ is independently selected from phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇.

In some embodiments of the formulae above, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, R₃ is (C₁-C₆)alkyl optionally substituted with one to three R₄, and each R₄ is independently selected from phenyl and 5- or 6-membered heteroaryl comprising 1 to 3 heteroatoms selected from O, N, and S, wherein the phenyl and heteroaryl groups are optionally substituted with one to three R₇. In another embodiment, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, R₃ is (C₁-C₆)alkyl substituted with one to three R₄, and each R₄ is independently selected from phenyl and 5- or 6-membered heteroaryl comprising 1 to 3 heteroatoms selected from O, N, and S, wherein the phenyl and heteroaryl groups are optionally substituted with one to three R₇.

In some embodiments of the formulae above, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, R₃ is (C₁-C₆)alkyl optionally substituted with one to three R₄, and each R₄ is phenyl optionally substituted with one to three R₇.

In another embodiment, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, R₃ is (C₁-C₆)alkyl substituted with one to three R₄, and each R₄ is phenyl optionally substituted with one to three R₇.

In some embodiments of the formulae above, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, R₃ is (C₁-C₆)alkyl optionally substituted with one to three R₄, and each R₄ is 5- or 6-membered heteroaryl comprising 1 to 3 heteroatoms selected from O, N, and S, optionally substituted with one to three R₇. In another embodiment, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, R₃ is (C₁-C₆)alkyl substituted with one to three R₄, and each R₄ is 5- or 6-membered heteroaryl comprising 1 to 3 heteroatoms selected from O, N, and S, optionally substituted with one to three R₇.

In some embodiments of the formulae above, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, R₃ is (C₁-C₆)alkyl optionally substituted with one to three R₄, and each R₄ is (C₃-C₈)cycloalkyl optionally substituted with one to three R₇. In another embodiment, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, R₃ is (C₁-C₆)alkyl substituted with one to three R₄, and each R₄ is (C₃-C₈)cycloalkyl optionally substituted with one to three R₇.

In some embodiments of the formulae above, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, R₃ is (C₁-C₆)alkyl optionally substituted with one to three R₄, and each R₄ is 5- to 7-membered heterocycloalkyl comprising 1 to 3 heteroatoms selected from O, N, and S, optionally substituted with one to three R₇. In another embodiment, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, R₃ is (C₁-C₆)alkyl substituted with one to three R₄, and each R₄ is 5- to 7-membered heterocycloalkyl comprising 1 to 3 heteroatoms selected from O, N, and S, optionally substituted with one to three R₇.

In some embodiments of the formulae above, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, and R₃ is (C₆-C₁₀)aryl, (C₃-C₈)cycloalkyl, or 5- to 7-membered heterocycloalkyl comprising 1 to 3 heteroatoms selected from O, N, and S, wherein the aryl, cycloalkyl, and heterocycloalkyl are optionally substituted with one to three R₅. In another embodiment, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, and R₃ is (C₆-C₁₀)aryl, (C₃-C₈)cycloalkyl, or 5- to 7-membered heterocycloalkyl comprising 1 to 3 heteroatoms selected from O, N, and S, wherein the aryl, cycloalkyl, and heterocycloalkyl are optionally substituted with one to three R₅.

In some embodiments of the formulae above, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, and R₃ is (C₆-C₁₀)aryl, (C₃-C₈)cycloalkyl, or 5- to 7-membered heterocycloalkyl comprising 1 to 3 heteroatoms selected from O, N, and S. In another embodiment, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, and R₃ is (C₆-C₁₀)aryl, (C₃-C₈)cycloalkyl, or 5- to 7-membered heterocycloalkyl comprising 1 to 3 heteroatoms selected from O, N, and S.

In some embodiments of the formulae above, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, and R₃ is (C₆-C₁₀)aryl, 5- or 6-membered heteroaryl comprising 1 to 3 heteroatoms selected from O, N, and S, or (C₃-C₈)cycloalkyl, wherein the aryl, heteroaryl and cycloalkyl are optionally substituted with one to three R₅. In another embodiment, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, and R₃ is (C₆-C₁₀)aryl, 5- or 6-membered heteroaryl comprising 1 to 3 heteroatoms selected from O, N, and S, or (C₃-C₈)cycloalkyl, wherein the aryl, heteroaryl and cycloalkyl are optionally substituted with one to three R₅.

In some embodiments of the formulae above, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, and R₃ is (C₆-C₁₀)aryl, 5- or 6-membered heteroaryl comprising 1 to 3 heteroatoms selected from O, N, and S, or (C₃-C₈)cycloalkyl. In another embodiment, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, and R₃ is (C₆-C₁₀)aryl, 5- or 6-membered heteroaryl comprising 1 to 3 heteroatoms selected from O, N, and S, or (C₃-C₈)cycloalkyl.

In some embodiments of the formulae above, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, and R₃ is (C₆-C₁₀)aryl optionally substituted with one to three R₅. In another embodiment, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, and R₃ is (C₆-C₁₀)aryl optionally substituted with one to three R₅.

In some embodiments of the formulae above, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, and R₃ is 5- or 6-membered heteroaryl comprising 1 to 3 heteroatoms selected from O, N, and S optionally substituted with one to three R₅. In another embodiment, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, and R₃ is 5- or 6-membered heteroaryl comprising 1 to 3 heteroatoms selected from O, N, and S optionally substituted with one to three R₅.

In some embodiments of the formulae above, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, and R₃ is (C₃-C₈)cycloalkyl optionally substituted with one to three R₅. In another embodiment, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, and R₃ is (C₃-C₈)cycloalkyl optionally substituted with one to three R₅.

In some embodiments of the formulae above, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, and R₃ is 5- to 7-membered heterocycloalkyl comprising 1 to 3 heteroatoms selected from O, N, and S, optionally substituted with one to three R₅. In another embodiment, Rₓ is H, n is 0, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, and R₃ is 5- to 7-membered heterocycloalkyl comprising 1 to 3 heteroatoms selected from O, N, and S, optionally substituted with one to three R₅.

In some embodiments of the formulae above, Rₓ is H, n is 1, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN. In another embodiment, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, and each R₂ is independently (C₁-C₆)alkyl. In yet another embodiment, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, and R₃ is (C₁-C₆)alkyl optionally substituted with one to three R₄. In another embodiment, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, and R₃ is (C₁-C₆)alkyl substituted with one to three R₄.

In some embodiments of the formulae above, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl optionally substituted with one to three R₄, and each R₄ is independently selected from —C(O)OR₆, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇. In another embodiment, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl substituted with one to three R₄, and each R₄ is independently selected from —C(O)OR₆, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In some embodiments of the formulae above, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl optionally substituted with one to three R₄, and each R₄ is independently selected from (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇. In another embodiment, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl substituted with one to three R₄, and each R₄ is independently selected from (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In some embodiments of the formulae above, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl optionally substituted with one to three R₄, and each R₄ is independently selected from halogen, —OH, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In another embodiment, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl substituted with one to three R₄, and each R₄ is independently selected from halogen, —OH, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In some embodiments of the formulae above, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl optionally substituted with one to three R₄, and each R₄ is independently selected from halogen, —OH, phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇.

In another embodiment, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl substituted with one to three R₄, and each R₄ is independently selected from halogen, —OH, phenyl, 5- or 6-membered heteroaryl comprising 1 to 3 heteroatoms selected from O, N, and S, (C₃-C₅)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇.

In some embodiments of the formulae above, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl optionally substituted with one to three R₄, and each R₄ is independently selected from phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇. In another embodiment, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl substituted with one to three R₄, and each R₄ is independently selected from phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇.

In some embodiments of the formulae above, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl optionally substituted with one to three R₄, and each R₄ is independently selected from phenyl and 5- or 6-membered heteroaryl comprising 1 to 3 heteroatoms selected from O, N, and S, wherein the phenyl and heteroaryl groups are optionally substituted with one to three R₇. In another embodiment, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl substituted with one to three R₄, and each R₄ is independently selected from phenyl and 5- or 6-membered heteroaryl comprising 1 to 3 heteroatoms selected from O, N, and S, wherein the phenyl and heteroaryl groups are optionally substituted with one to three R₇.

In some embodiments of the formulae above, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl optionally substituted with one to three R₄, and each R₄ is phenyl optionally substituted with one to three R₇. In another embodiment, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl substituted with one to three R₄, and each R₄ is phenyl optionally substituted with one to three R₇.

In some embodiments of the formulae above, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl optionally substituted with one to three R₄, and each R₄ is 5- or 6-membered heteroaryl comprising 1 to 3 heteroatoms selected from O, N, and S, optionally substituted with one to three R₇. In another embodiment, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl substituted with one to three R₄, and each R₄ is 5- or 6-membered heteroaryl comprising 1 to 3 heteroatoms selected from O, N, and S, optionally substituted with one to three R₇.

In some embodiments of the formulae above, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl optionally substituted with one to three R₄, and each R₄ is (C₃-C₈)cycloalkyl optionally substituted with one to three R₇. In another embodiment, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl substituted with one to three R₄, and each R₄ is (C₃-C₈)cycloalkyl optionally substituted with one to three R₇.

In some embodiments of the formulae above, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl optionally substituted with one to three R₄, and each R₄ is 5- to 7-membered heterocycloalkyl comprising 1 to 3 heteroatoms selected from O, N, and S, optionally substituted with one to three R₇. In another embodiment, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl substituted with one to three R₄, and each R₄ is 5- to 7-membered heterocycloalkyl comprising 1 to 3 heteroatoms selected from O, N, and S, optionally substituted with one to three R₇.

In some embodiments of the formulae above, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, and R₃ is (C₆-C₁₀)aryl, (C₃-C₈)cycloalkyl, or 5- to 7-membered heterocycloalkyl comprising 1 to 3 heteroatoms selected from O, N, and S, wherein the aryl, cycloalkyl, and heterocycloalkyl are optionally substituted with one to three R₅.

In some embodiments of the formulae above, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, and R₃ is (C₆-C₁₀)aryl, (C₃-C₈)cycloalkyl, or 5- to 7-membered heterocycloalkyl comprising 1 to 3 heteroatoms selected from O, N, and S.

In some embodiments of the formulae above, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, and R₃ is (C₆-C₁₀)aryl, 5- or 6-membered heteroaryl comprising 1 to 3 heteroatoms selected from O, N, and S, or (C₃-C₈)cycloalkyl, wherein the aryl, heteroaryl and cycloalkyl are optionally substituted with one to three R₅.

In some embodiments of the formulae above, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, and R₃ is (C₆-C₁₀)aryl, 5- or 6-membered heteroaryl comprising 1 to 3 heteroatoms selected from O, N, and S, or (C₃-C₈)cycloalkyl.

In some embodiments of the formulae above, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, and R₃ is (C₆-C₁₀)aryl optionally substituted with one to three R₅.

In some embodiments of the formulae above, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, and R₃ is 5- or 6-membered heteroaryl comprising 1 to 3 heteroatoms selected from O, N, and S optionally substituted with one to three R₅.

In some embodiments of the formulae above, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, and R₃ is (C₃-C₈)cycloalkyl optionally substituted with one to three R₅.

In some embodiments of the formulae above, Rₓ is H, n is 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, and R₃ is 5- to 7-membered heterocycloalkyl comprising 1 to 3 heteroatoms selected from O, N, and S, optionally substituted with one to three R₅.

In some embodiments of the formulae above, Rₓ is H, n is 0 or 1, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN. In another embodiment, Rₓ is H, n is 0 or 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, and each R₂ is independently (C₁-C₆)alkyl. In another embodiment, Rₓ is H, n is 0 or 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, and R₃ is (C₁-C₆)alkyl optionally substituted with one to three R₄. In another embodiment, Rₓ is H, n is 0 or 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, and R₃ is (C₁-C₆)alkyl substituted with one to three R₄.

In some embodiments of the formulae above, Rₓ is H, n is 0 or 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl optionally substituted with one to three R₄, and each R₄ is independently selected from —C(O)OR₆, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In another embodiment, Rₓ is H, n is 0 or 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl substituted with one to three R₄, and each R₄ is independently selected from —C(O)OR₆, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In some embodiments of the formulae above, Rₓ is H, n is 0 or 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl optionally substituted with one to three R₄, and each R₄ is independently selected from (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇. In another embodiment, Rₓ is H, n is 0 or 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl substituted with one to three R₄, and each R₄ is independently selected from (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In some embodiments of the formulae above, Rₓ is H, n is 0 or 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl optionally substituted with one to three R₄, and each R₄ is independently selected from halogen, —OH, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In another embodiment, Rₓ is H, n is 0 or 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl substituted with one to three R₄, and each R₄ is independently selected from halogen, —OH, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In some embodiments of the formulae above, Rₓ is H, n is 0 or 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl optionally substituted with one to three R₄, and each R₄ is independently selected from halogen, —OH, phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇.

In another embodiment, Rₓ is H, n is 0 or 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl substituted with one to three R₄, and each R₄ is independently selected from halogen, —OH, phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇.

In some embodiments of the formulae above, Rₓ is H, n is 0 or 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl optionally substituted with one to three R₄, and each R₄ is independently selected from phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇. In another embodiment, Rₓ is H, n is 0 or 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl substituted with one to three R₄, and each R₄ is independently selected from phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇.

In some embodiments of the formulae above, Rₓ is H, n is 0 or 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl optionally substituted with one to three R₄, and each R₄ is independently selected from phenyl and 5- or 6-membered heteroaryl comprising 1 to 3 heteroatoms selected from O, N, and S, wherein the phenyl and heteroaryl groups are optionally substituted with one to three R₇. In another embodiment, Rₓ is H, n is 0 or 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl substituted with one to three R₄, and each R₄ is independently selected from phenyl and 5- or 6-membered heteroaryl comprising 1 to 3 heteroatoms selected from O, N, and S, wherein the phenyl and heteroaryl groups are optionally substituted with one to three R₇.

In some embodiments of the formulae above, Rₓ is H, n is 0 or 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl optionally substituted with one to three R₄, and each R₄ is phenyl optionally substituted with one to three R₇. In another embodiment, Rₓ is H, n is 0 or 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl substituted with one to three R₄, and each R₄ is phenyl optionally substituted with one to three R₇.

In some embodiments of the formulae above, Rₓ is H, n is 0 or 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl optionally substituted with one to three R₄, and each R₄ is 5- or 6-membered heteroaryl comprising 1 to 3 heteroatoms selected from O, N, and S, optionally substituted with one to three R₇. In another embodiment, Rₓ is H, n is 0 or 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl substituted with one to three R₄, and each R₄ is 5- or 6-membered heteroaryl comprising 1 to 3 heteroatoms selected from O, N, and S, optionally substituted with one to three R₇.

In some embodiments of the formulae above, Rₓ is H, n is 0 or 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl optionally substituted with one to three R₄, and each R₄ is (C₃-C₈)cycloalkyl optionally substituted with one to three R₇. In another embodiment, Rₓ is H, n is 0 or 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl substituted with one to three R₄, and each R₄ is (C₃-C₈)cycloalkyl optionally substituted with one to three R₇.

In some embodiments of the formulae above, Rₓ is H, n is 0 or 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl optionally substituted with one to three R₄, and each R₄ is 5- to 7-membered heterocycloalkyl comprising 1 to 3 heteroatoms selected from O, N, and S, optionally substituted with one to three R₇. In another embodiment, Rₓ is H, n is 0 or 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl substituted with one to three R₄, and each R₄ is 5- to 7-membered heterocycloalkyl comprising 1 to 3 heteroatoms selected from O, N, and S, optionally substituted with one to three R₇.

In some embodiments of the formulae above, Rₓ is H, n is 0 or 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, and R₃ is (C₆-C₁₀)aryl, (C₃-C₈)cycloalkyl, or 5- to 7-membered heterocycloalkyl comprising 1 to 3 heteroatoms selected from O, N, and S, wherein the aryl, cycloalkyl, and heterocycloalkyl are optionally substituted with one to three R₅. In another embodiment, Rₓ is H, n is 0 or 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, and R₃ is (C₆-C₁₀)aryl, (C₃-C₈)cycloalkyl, or 5- to 7-membered heterocycloalkyl comprising 1 to 3 heteroatoms selected from O, N, and S, wherein the aryl, cycloalkyl, and heterocycloalkyl are optionally substituted with one to three R₅.

In some embodiments of the formulae above, Rₓ is H, n is 0 or 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, and R₃ is (C₆-C₁₀)aryl, (C₃-C₈)cycloalkyl, or 5- to 7-membered heterocycloalkyl comprising 1 to 3 heteroatoms selected from O, N, and S. In another embodiment, Rₓ is H, n is 0 or 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, and R₃ is (C₆-C₁₀)aryl, (C₃-C₈)cycloalkyl, or 5- to 7-membered heterocycloalkyl comprising 1 to 3 heteroatoms selected from O, N, and S.

In some embodiments of the formulae above, Rₓ is H, n is 0 or 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, and R₃ is (C₆-C₁₀)aryl, 5- or 6-membered heteroaryl comprising 1 to 3 heteroatoms selected from O, N, and S, or (C₃-C₈)cycloalkyl, wherein the aryl, heteroaryl and cycloalkyl are optionally substituted with one to three R₅. In another embodiment, Rₓ is H, n is 0 or 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, and R₃ is (C₆-C₁₀)aryl, 5- or 6-membered heteroaryl comprising 1 to 3 heteroatoms selected from O, N, and S, or (C₃-C₈)cycloalkyl, wherein the aryl, heteroaryl and cycloalkyl are optionally substituted with one to three R₅.

In some embodiments of the formulae above, Rₓ is H, n is 0 or 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, and R₃ is (C₆-C₁₀)aryl, 5- or 6-membered heteroaryl comprising 1 to 3 heteroatoms selected from O, N, and S, or (C₃-C₈)cycloalkyl. In another embodiment, Rₓ is H, n is 0 or 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, and R₃ is (C₆-C₁₀)aryl, 5- or 6-membered heteroaryl comprising 1 to 3 heteroatoms selected from O, N, and S, or (C₃-C₈)cycloalkyl.

In some embodiments of the formulae above, Rₓ is H, n is 0 or 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, and R₃ is (C₆-C₁₀)aryl optionally substituted with one to three R₅. In another embodiment, Rₓ is H, n is 0 or 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, and R₃ is (C₆-C₁₀)aryl optionally substituted with one to three R₅.

In some embodiments of the formulae above, Rₓ is H, n is 0 or 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, and R₃ is 5- or 6-membered heteroaryl comprising 1 to 3 heteroatoms selected from O, N, and S optionally substituted with one to three R₅. In another embodiment, Rₓ is H, n is 0 or 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, and R₃ is 5- or 6-membered heteroaryl comprising 1 to 3 heteroatoms selected from O, N, and S optionally substituted with one to three R₅.

In some embodiments of the formulae above, Rₓ is H, n is 0 or 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, and R₃ is (C₃-C₈)cycloalkyl optionally substituted with one to three R₅. In another embodiment, Rₓ is H, n is 0 or 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, and R₃ is (C₃-C₈)cycloalkyl optionally substituted with one to three R₅.

In some embodiments of the formulae above, Rₓ is H, n is 0 or 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, and R₃ is 5- to 7-membered heterocycloalkyl comprising 1 to 3 heteroatoms selected from O, N, and S, optionally substituted with one to three R₅. In another embodiment, Rₓ is H, n is 0 or 1, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, and R₃ is 5- to 7-membered heterocycloalkyl comprising 1 to 3 heteroatoms selected from O, N, and S, optionally substituted with one to three R₅.

In some embodiments of the formulae above, Rₓ is H, n is 0, 1, or 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN. In another embodiment, Rₓ is H, n is 0, 1, or 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, and each R₂ is independently (C₁-C₆)alkyl. In another embodiment, Rₓ is H, n is 0, 1, or 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, and R₃ is (C₁-C₆)alkyl optionally substituted with one to three R₄. In another embodiment, Rₓ is H, n is 0, 1, or 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, and R₃ is (C₁-C₆)alkyl substituted with one to three R₄.

In some embodiments of the formulae above, Rₓ is H, n is 0, 1, or 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl optionally substituted with one to three R₄, and each R₄ is independently selected from —C(O)OR₆, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In another embodiment, Rₓ is H, n is 0, 1, or 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl substituted with one to three R₄, and each R₄ is independently selected from —C(O)OR₆, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In some embodiments of the formulae above, Rₓ is H, n is 0, 1, or 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl optionally substituted with one to three R₄, and each R₄ is independently selected from (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In another embodiment, Rₓ is H, n is 0, 1, or 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl substituted with one to three R₄, and each R₄ is independently selected from (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In some embodiments of the formulae above, Rₓ is H, n is 0, 1, or 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl optionally substituted with one to three R₄, and each R₄ is independently selected from halogen, —OH, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In another embodiment, Rₓ is H, n is 0, 1, or 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl substituted with one to three R₄, and each R₄ is independently selected from halogen, —OH, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In some embodiments of the formulae above, Rₓ is H, n is 0, 1, or 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl optionally substituted with one to three R₄, and each R₄ is independently selected from halogen, —OH, phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇.

In another embodiment, Rₓ is H, n is 0, 1, or 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl substituted with one to three R₄, and each R₄ is independently selected from halogen, —OH, phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇.

In some embodiments of the formulae above, Rₓ is H, n is 0, 1, or 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl optionally substituted with one to three R₄, and each R₄ is independently selected from phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇. In another embodiment, Rₓ is H, n is 0, 1, or 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl substituted with one to three R₄, and each R₄ is independently selected from phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇.

In some embodiments of the formulae above, Rₓ is H, n is 0, 1, or 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl optionally substituted with one to three R₄, and each R₄ is independently selected from phenyl and 5- or 6-membered heteroaryl comprising 1 to 3 heteroatoms selected from O, N, and S, wherein the phenyl and heteroaryl groups are optionally substituted with one to three R₇. In another embodiment, Rₓ is H, n is 0, 1, or 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl substituted with one to three R₄, and each R₄ is independently selected from phenyl and 5- or 6-membered heteroaryl comprising 1 to 3 heteroatoms selected from O, N, and S, wherein the phenyl and heteroaryl groups are optionally substituted with one to three R₇.

In some embodiments of the formulae above, Rₓ is H, n is 0, 1, or 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl optionally substituted with one to three R₄, and each R₄ is phenyl optionally substituted with one to three R₇. In another embodiment, Rₓ is H, n is 0, 1, or 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl substituted with one to three R₄, and each R₄ is phenyl optionally substituted with one to three R₇.

In some embodiments of the formulae above, Rₓ is H, n is 0, 1, or 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl optionally substituted with one to three R₄, and each R₄ is 5- or 6-membered heteroaryl comprising 1 to 3 heteroatoms selected from O, N, and S, optionally substituted with one to three R₇. In another embodiment, Rₓ is H, n is 0, 1, or 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl substituted with one to three R₄, and each R₄ is 5- or 6-membered heteroaryl comprising 1 to 3 heteroatoms selected from O, N, and S, optionally substituted with one to three R₇.

In some embodiments of the formulae above, Rₓ is H, n is 0, 1, or 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl optionally substituted with one to three R₄, and each R₄ is (C₃-C₈)cycloalkyl optionally substituted with one to three R₇. In another embodiment, Rₓ is H, n is 0, 1, or 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl substituted with one to three R₄, and each R₄ is (C₃-C₈)cycloalkyl optionally substituted with one to three R₇.

In some embodiments of the formulae above, Rₓ is H, n is 0, 1, or 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl optionally substituted with one to three R₄, and each R₄ is 5- to 7-membered heterocycloalkyl comprising 1 to 3 heteroatoms selected from O, N, and S, optionally substituted with one to three R₇. In another embodiment, Rₓ is H, n is 0, 1, or 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl substituted with one to three R₄, and each R₄ is 5- to 7-membered heterocycloalkyl comprising 1 to 3 heteroatoms selected from O, N, and S, optionally substituted with one to three R₇.

In some embodiments of the formulae above, Rₓ is H, n is 0, 1, or 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, and R₃ is (C₆-C₁₀)aryl, (C₃-C₈)cycloalkyl, or 5- to 7-membered heterocycloalkyl comprising 1 to 3 heteroatoms selected from O, N, and S, wherein the aryl, cycloalkyl, and heterocycloalkyl are optionally substituted with one to three R₅.

In some embodiments of the formulae above, Rₓ is H, n is 0, 1, or 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, and R₃ is (C₆-C₁₀)aryl, (C₃-C₈)cycloalkyl, or 5- to 7-membered heterocycloalkyl comprising 1 to 3 heteroatoms selected from O, N, and S.

In some embodiments of the formulae above, Rₓ is H, n is 0, 1, or 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, and R₃ is (C₆-C₁₀)aryl, 5- or 6-membered heteroaryl comprising 1 to 3 heteroatoms selected from O, N, and S, or (C₃-C₈)cycloalkyl, wherein the aryl, heteroaryl and cycloalkyl are optionally substituted with one to three R₅.

In some embodiments of the formulae above, Rₓ is H, n is 0, 1, or 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, and R₃ is (C₆-C₁₀)aryl, 5- or 6-membered heteroaryl comprising 1 to 3 heteroatoms selected from O, N, and S, or (C₃-C₈)cycloalkyl.

In some embodiments of the formulae above, Rₓ is H, n is 0, 1, or 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, and R₃ is (C₆-C₁₀)aryl optionally substituted with one to three R₅.

In some embodiments of the formulae above, Rₓ is H, n is 0, 1, or 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, and R₃ is 5- or 6-membered heteroaryl comprising 1 to 3 heteroatoms selected from O, N, and S optionally substituted with one to three R₅.

In some embodiments of the formulae above, Rₓ is H, n is 0, 1, or 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, and R₃ is (C₃-C₈)cycloalkyl optionally substituted with one to three R₅.

In some embodiments of the formulae above, Rₓ is H, n is 0, 1, or 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, and R₃ is 5- to 7-membered heterocycloalkyl comprising 1 to 3 heteroatoms selected from O, N, and S, optionally substituted with one to three R₅.

In some embodiments of the formulae above, Rₓ is H, n is 1 or 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN. In another embodiment, Rₓ is H, n is 1 or 2, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, and each R₂ is independently (C₁-C₆)alkyl. In another embodiment, Rₓ is H, n is 1 or 2, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, and R₃ is (C₁-C₆)alkyl optionally substituted with one to three R₄. In yet another embodiment, Rₓ is H, n is 1 or 2, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, and R₃ is (C₁-C₆)alkyl substituted with one to three R₄.

In some embodiments of the formulae above, Rₓ is H, n is 1 or 2, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl optionally substituted with one to three R₄, and each R₄ is independently selected from —C(O)OR₆, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In another embodiment, Rₓ is H, n is 1 or 2, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl substituted with one to three R₄, and each R₄ is independently selected from —C(O)OR₆, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In some embodiments of the formulae above, Rₓ is H, n is 1 or 2, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl optionally substituted with one to three R₄, and each R₄ is independently selected from (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇. In another embodiment, Rₓ is H, n is 1 or 2, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl substituted with one to three R₄, and each R₄ is independently selected from (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In some embodiments of the formulae above, Rₓ is H, n is 1 or 2, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl optionally substituted with one to three R₄, and each R₄ is independently selected from halogen, —OH, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In another embodiment, Rₓ is H, n is 1 or 2, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl substituted with one to three R₄, and each R₄ is independently selected from halogen, —OH, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In some embodiments of the formulae above, Rₓ is H, n is 1 or 2, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl optionally substituted with one to three R₄, and each R₄ is independently selected from halogen, —OH, phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇.

In another embodiment, Rₓ is H, n is 1 or 2, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl substituted with one to three R₄, and each R₄ is independently selected from halogen, —OH, phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇.

In some embodiments of the formulae above, Rₓ is H, n is 1 or 2, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂ NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl optionally substituted with one to three R₄, and each R₄ is independently selected from phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇. In another embodiment, Rₓ is H, n is 1 or 2, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl substituted with one to three R₄, and each R₄ is independently selected from phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇.

In some embodiments of the formulae above, Rₓ is H, n is 1 or 2, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl optionally substituted with one to three R₄, and each R₄ is independently selected from phenyl and 5- or 6-membered heteroaryl comprising 1 to 3 heteroatoms selected from O, N, and S, wherein the phenyl and heteroaryl groups are optionally substituted with one to three R₇. In another embodiment, Rₓ is H, n is 1 or 2, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl substituted with one to three R₄, and each R₄ is independently selected from phenyl and 5- or 6-membered heteroaryl comprising 1 to 3 heteroatoms selected from O, N, and S, wherein the phenyl and heteroaryl groups are optionally substituted with one to three R₇.

In some embodiments of the formulae above, Rₓ is H, n is 1 or 2, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl optionally substituted with one to three R₄, and each R₄ is phenyl optionally substituted with one to three R₇. In another embodiment, Rₓ is H, n is 1 or 2, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl substituted with one to three R₄, and each R₄ is phenyl optionally substituted with one to three R₇.

In some embodiments of the formulae above, Rₓ is H, n is 1 or 2, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl optionally substituted with one to three R₄, and each R₄ is 5- or 6-membered heteroaryl comprising 1 to 3 heteroatoms selected from O, N, and S, optionally substituted with one to three R₇. In another embodiment, Rₓ is H, n is 1 or 2, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl substituted with one to three R₄, and each R₄ is 5- or 6-membered heteroaryl comprising 1 to 3 heteroatoms selected from O, N, and S, optionally substituted with one to three R₇.

In some embodiments of the formulae above, Rₓ is H, n is 1 or 2, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl optionally substituted with one to three R₄, and each R₄ is (C₃-C₈)cycloalkyl optionally substituted with one to three R₇. In another embodiment, Rₓ is H, n is 1 or 2, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl substituted with one to three R₄, and each R₄ is (C₃-C₈)cycloalkyl optionally substituted with one to three R₇.

In some embodiments of the formulae above, Rₓ is H, n is 1 or 2, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl optionally substituted with one to three R₄, and each R₄ is 5- to 7-membered heterocycloalkyl comprising 1 to 3 heteroatoms selected from O, N, and S, optionally substituted with one to three R₇. In another embodiment, Rₓ is H, n is 1 or 2, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, R₃ is (C₁-C₆)alkyl substituted with one to three R₄, and each R₄ is 5- to 7-membered heterocycloalkyl comprising 1 to 3 heteroatoms selected from O, N, and S, optionally substituted with one to three R₇.

In some embodiments of the formulae above, Rₓ is H, n is 1 or 2, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, and R₃ is (C₆-C₁₀)aryl, (C₃-C₈)cycloalkyl, or 5- to 7-membered heterocycloalkyl comprising 1 to 3 heteroatoms selected from O, N, and S, wherein the aryl, cycloalkyl, and heterocycloalkyl are optionally substituted with one to three R₅.

In some embodiments of the formulae above, Rₓ is H, n is 1 or 2, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, and R₃ is (C₆-C₁₀)aryl, (C₃-C₈)cycloalkyl, or 5- to 7-membered heterocycloalkyl comprising 1 to 3 heteroatoms selected from O, N, and S.

In some embodiments of the formulae above, Rₓ is H, n is 1 or 2, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, and R₃ is (C₆-C₁₀)aryl, 5- or 6-membered heteroaryl comprising 1 to 3 heteroatoms selected from O, N, and S, or (C₃-C₈)cycloalkyl, wherein the aryl, heteroaryl and cycloalkyl are optionally substituted with one to three R₅.

In some embodiments of the formulae above, Rₓ is H, n is 1 or 2, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, and R₃ is (C₆-C₁₀)aryl, 5- or 6-membered heteroaryl comprising 1 to 3 heteroatoms selected from O, N, and S, or (C₃-C₈)cycloalkyl.

In some embodiments of the formulae above, Rₓ is H, n is 1 or 2, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, and R₃ is (C₆-C₁₀)aryl optionally substituted with one to three R₅.

In some embodiments of the formulae above, Rₓ is H, n is 1 or 2, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, and R₃ is 5- or 6-membered heteroaryl comprising 1 to 3 heteroatoms selected from O, N, and S optionally substituted with one to three R₅.

In some embodiments of the formulae above, Rₓ is H, n is 1 or 2, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂ NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, and R₃ is (C₃-C₈)cycloalkyl optionally substituted with one to three R₅.

In some embodiments of the formulae above, Rₓ is H, n is 1 or 2, R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, and R₃ is 5- to 7-membered heterocycloalkyl comprising 1 to 3 heteroatoms selected from O, N, and S, optionally substituted with one to three R₅.

In some embodiments of the formulae above, Rₓ is H, n is 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN. In another embodiment, Rₓ is H, n is 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, and each R₂ is independently (C₁-C₆)alkyl. In another embodiment, Rₓ is H, n is 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl. and R₃ is (C₁-C₆)alkyl optionally substituted with one to three R₄. In yet another embodiment, Rₓ is H, n is 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, and R₃ is (C₁-C₆)alkyl substituted with one to three R₄.

In some embodiments of the formulae above, Rₓ is H, n is 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl. R₃ is (C₁-C₆)alkyl optionally substituted with one to three R₄, and each R₄ is independently selected from —C(O)OR₆, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In another embodiment, Rₓ is H, n is 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl. R₃ is (C₁-C₆)alkyl substituted with one to three R₄, and each R₄ is independently selected from —C(O)OR₆, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In some embodiments of the formulae above, Rₓ is H, n is 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl. R₃ is (C₁-C₆)alkyl optionally substituted with one to three R₄, and each R₄ is independently selected from (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇. In another embodiment, Rₓ is H, n is 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl. R₃ is (C₁-C₆)alkyl substituted with one to three R₄, and each R₄ is independently selected from (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In some embodiments of the formulae above, Rₓ is H, n is 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl. R₃ is (C₁-C₆)alkyl optionally substituted with one to three R₄, and each R₄ is independently selected from halogen, —OH, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In another embodiment, Rₓ is H, n is 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl. R₃ is (C₁-C₆)alkyl substituted with one to three R₄, and each R₄ is independently selected from halogen, —OH, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In some embodiments of the formulae above, Rₓ is H, n is 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl. R₃ is (C₁-C₆)alkyl optionally substituted with one to three R₄, and each R₄ is independently selected from halogen, —OH, phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇.

In another embodiment, Rₓ is H, n is 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl. R₃ is (C₁-C₆)alkyl substituted with one to three R₄, and each R₄ is independently selected from halogen, —OH, phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇.

In some embodiments of the formulae above, Rₓ is H, n is 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl. R₃ is (C₁-C₆)alkyl optionally substituted with one to three R₄, and each R₄ is independently selected from phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇. In another embodiment, Rₓ is H, n is 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl. R₃ is (C₁-C₆)alkyl substituted with one to three R₄, and each R₄ is independently selected from phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇.

In some embodiments of the formulae above, Rₓ is H, n is 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl. R₃ is (C₁-C₆)alkyl optionally substituted with one to three R₄, and each R₄ is independently selected from phenyl and 5- or 6-membered heteroaryl comprising 1 to 3 heteroatoms selected from O, N, and S, wherein the phenyl and heteroaryl groups are optionally substituted with one to three R₇. In another embodiment, Rₓ is H, n is 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl. R₃ is (C₁-C₆)alkyl substituted with one to three R₄, and each R₄ is independently selected from phenyl and 5- or 6-membered heteroaryl comprising 1 to 3 heteroatoms selected from O, N, and S, wherein the phenyl and heteroaryl groups are optionally substituted with one to three R₇.

In some embodiments of the formulae above, Rₓ is H, n is 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl. R₃ is (C₁-C₆)alkyl optionally substituted with one to three R₄, and each R₄ is phenyl optionally substituted with one to three R₇. In another embodiment, Rₓ is H, n is 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl. R₃ is (C₁-C₆)alkyl substituted with one to three R₄, and each R₄ is phenyl optionally substituted with one to three R₇.

In some embodiments of the formulae above, Rₓ is H, n is 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl. R₃ is (C₁-C₆)alkyl optionally substituted with one to three R₄, and each R₄ is 5- or 6-membered heteroaryl comprising 1 to 3 heteroatoms selected from O, N, and S, optionally substituted with one to three R₇. In another embodiment, Rₓ is H, n is 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl. R₃ is (C₁-C₆)alkyl substituted with one to three R₄, and each R₄ is 5- or 6-membered heteroaryl comprising 1 to 3 heteroatoms selected from O, N, and S, optionally substituted with one to three R₇.

In some embodiments of the formulae above, Rₓ is H, n is 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl. R₃ is (C₁-C₆)alkyl optionally substituted with one to three R₄, and each R₄ is (C₃-C₈)cycloalkyl optionally substituted with one to three R₇. In another embodiment, Rₓ is H, n is 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl. R₃ is (C₁-C₆)alkyl substituted with one to three R₄, and each R₄ is (C₃-C₈)cycloalkyl optionally substituted with one to three R₇.

In some embodiments of the formulae above, Rₓ is H, n is 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl. R₃ is (C₁-C₆)alkyl optionally substituted with one to three R₄, and each R₄ is 5- to 7-membered heterocycloalkyl comprising 1 to 3 heteroatoms selected from O, N, and S, optionally substituted with one to three R₇. In another embodiment, Rₓ is H, n is 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl. R₃ is (C₁-C₆)alkyl substituted with one to three R₄, and each R₄ is 5- to 7-membered heterocycloalkyl comprising 1 to 3 heteroatoms selected from O, N, and S, optionally substituted with one to three R₇.

In some embodiments of the formulae above, Rₓ is H, n is 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl. and R₃ is (C₆-C₁₀)aryl, (C₃-C₈)cycloalkyl, or 5- to 7-membered heterocycloalkyl comprising 1 to 3 heteroatoms selected from O, N, and S, wherein the aryl, cycloalkyl, and heterocycloalkyl are optionally substituted with one to three R₅.

In some embodiments of the formulae above, Rₓ is H, n is 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl. and R₃ is (C₆-C₁₀)aryl, (C₃-C₈)cycloalkyl, or 5- to 7-membered heterocycloalkyl comprising 1 to 3 heteroatoms selected from O, N, and S.

In some embodiments of the formulae above, Rₓ is H, n is 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, and R₃ is (C₆-C₁₀)aryl, 5- or 6-membered heteroaryl comprising 1 to 3 heteroatoms selected from O, N, and S, or (C₃-C₈)cycloalkyl, wherein the aryl, heteroaryl and cycloalkyl are optionally substituted with one to three R₅.

In some embodiments of the formulae above, Rₓ is H, n is 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, and R₃ is (C₆-C₁₀)aryl, 5- or 6-membered heteroaryl comprising 1 to 3 heteroatoms selected from O, N, and S, or (C₃-C₈)cycloalkyl.

In some embodiments of the formulae above, Rₓ is H, n is 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, and R₃ is (C₆-C₁₀)aryl optionally substituted with one to three R₅.

In some embodiments of the formulae above, Rₓ is H, n is 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, and R₃ is 5- or 6-membered heteroaryl comprising 1 to 3 heteroatoms selected from O, N, and S optionally substituted with one to three R₅.

In some embodiments of the formulae above, Rₓ is H, n is 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, and R₃ is (C₃-C₈)cycloalkyl optionally substituted with one to three R₅.

In some embodiments of the formulae above, Rₓ is H, n is 2, and R₁ is (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, each R₂ is independently (C₁-C₆)alkyl, and R₃ is 5- to 7-membered heterocycloalkyl comprising 1 to 3 heteroatoms selected from O, N, and S, optionally substituted with one to three R₅.

In some embodiments of the formulae above, Rₓ is H, n is 0, and R₁ is (C₁-C₆)alkoxy, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN. In another embodiment, Rₓ is H, n is 0, R₁ is (C₁-C₆)alkoxy, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, and R₃ is (C₁-C₆)alkyl optionally substituted with one to three R₄. In another embodiment, Rₓ is H, n is 0, R₁ is (C₁-C₆)alkoxy, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, and R₃ is (C₁-C₆)alkyl substituted with one to three R₄.

In some embodiments of the formulae above, Rₓ is H, n is 0, R₁ is (C₁-C₆)alkoxy, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, R₃ is (C₁-C₆)alkyl optionally substituted with one to three R₄, and each R₄ is independently selected from —C(O)OR₆, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇. In another embodiment, Rₓ is H, n is 0, R₁ is (C₁-C₆)alkoxy, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, R₃ is (C₁-C₆)alkyl substituted with one to three R₄, and each R₄ is independently selected from —C(O)OR₆, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In some embodiments of the formulae above, Rₓ is H, n is 0, R₁ is (C₁-C₆)alkoxy, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, R₃ is (C₁-C₆)alkyl optionally substituted with one to three R₄, and each R₄ is independently selected from (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇. In another embodiment, Rₓ is H, n is 0, R₁ is (C₁-C₆)alkoxy, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, R₃ is (C₁-C₆)alkyl substituted with one to three R₄, and each R₄ is independently selected from (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In some embodiments of the formulae above, Rₓ is H, n is 0, R₁ is (C₁-C₆)alkoxy, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, R₃ is (C₁-C₆)alkyl optionally substituted with one to three R₄, and each R₄ is independently selected from halogen, —OH, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇. In another embodiment, Rₓ is H, n is 0, R₁ is (C₁-C₆)alkoxy, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, R₃ is (C₁-C₆)alkyl substituted with one to three R₄, and each R₄ is independently selected from halogen, —OH, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In some embodiments of the formulae above, Rₓ is H, n is 0, R₁ is (C₁-C₆)alkoxy, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, R₃ is (C₁-C₆)alkyl optionally substituted with one to three R₄, and each R₄ is independently selected from halogen, —OH, phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇. In another embodiment, Rₓ is H, n is 0, R₁ is (C₁-C₆)alkoxy, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, R₃ is (C₁-C₆)alkyl substituted with one to three R₄, and each R₄ is independently selected from halogen, —OH, phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇.

In some embodiments of the formulae above, Rₓ is H, n is 0, R₁ is (C₁-C₆)alkoxy, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, R₃ is (C₁-C₆)alkyl optionally substituted with one to three R₄, and each R₄ is independently selected from phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇. In another embodiment, Rₓ is H, n is 0, R₁ is (C₁-C₆)alkoxy, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, R₃ is (C₁-C₆)alkyl substituted with one to three R₄, and each R₄ is independently selected from phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇.

In some embodiments of the formulae above, Rₓ is H, n is 0, R₁ is (C₁-C₆)alkoxy, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, R₃ is (C₁-C₆)alkyl optionally substituted with one to three R₄, and each R₄ is independently selected from phenyl and 5- or 6-membered heteroaryl comprising 1 to 3 heteroatoms selected from O, N, and S, wherein the phenyl and heteroaryl groups are optionally substituted with one to three R₇. In another embodiment, Rₓ is H, n is 0, R₁ is (C₁-C₆)alkoxy, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, R₃ is (C₁-C₆)alkyl substituted with one to three R₄, and each R₄ is independently selected from phenyl and 5- or 6-membered heteroaryl comprising 1 to 3 heteroatoms selected from O, N, and S, wherein the phenyl and heteroaryl groups are optionally substituted with one to three R₇.

In some embodiments of the formulae above, Rₓ is H, n is 0, R₁ is (C₁-C₆)alkoxy, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, R₃ is (C₁-C₆)alkyl optionally substituted with one to three R₄, and each R₄ is phenyl optionally substituted with one to three R₇. In another embodiment, Rₓ is H, n is 0, R₁ is (C₁-C₆)alkoxy, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, R₃ is (C₁-C₆)alkyl substituted with one to three R₄, and each R₄ is phenyl optionally substituted with one to three R₇.

In some embodiments of the formulae above, Rₓ is H, n is 0, R₁ is (C₁-C₆)alkoxy, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, R₃ is (C₁-C₆)alkyl optionally substituted with one to three R₄, and each R₄ is 5- or 6-membered heteroaryl comprising 1 to 3 heteroatoms selected from O, N, and S, optionally substituted with one to three R₇. In another embodiment, Rₓ is H, n is 0, R₁ is (C₁-C₆)alkoxy, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, R₃ is (C₁-C₆)alkyl substituted with one to three R₄, and each R₄ is 5- or 6-membered heteroaryl comprising 1 to 3 heteroatoms selected from O, N, and S, optionally substituted with one to three R₇.

In some embodiments of the formulae above, Rₓ is H, n is 0, R₁ is (C₁-C₆)alkoxy, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, R₃ is (C₁-C₆)alkyl optionally substituted with one to three R₄, and each R₄ is (C₃-C₈)cycloalkyl optionally substituted with one to three R₇. In another embodiment, Rₓ is H, n is 0, R₁ is (C₁-C₆)alkoxy, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, R₃ is (C₁-C₆)alkyl substituted with one to three R₄, and each R₄ is (C₃-C₈)cycloalkyl optionally substituted with one to three R₇.

In some embodiments of the formulae above, Rₓ is H, n is 0, R₁ is (C₁-C₆)alkoxy, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, R₃ is (C₁-C₆)alkyl optionally substituted with one to three R₄, and each R₄ is 5- to 7-membered heterocycloalkyl comprising 1 to 3 heteroatoms selected from O, N, and S, optionally substituted with one to three R₇. In another embodiment, Rₓ is H, n is 0, R₁ is (C₁-C₆)alkoxy, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, R₃ is (C₁-C₆)alkyl substituted with one to three R₄, and each R₄ is 5- to 7-membered heterocycloalkyl comprising 1 to 3 heteroatoms selected from O, N, and S, optionally substituted with one to three R₇.

In some embodiments of the formulae above, Rₓ is H, n is 0, R₁ is (C₁-C₆)alkoxy, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, and R₃ is (C₆-C₁₀)aryl, (C₃-C₈)cycloalkyl, or 5- to 7-membered heterocycloalkyl comprising 1 to 3 heteroatoms selected from O, N, and S, wherein the aryl, cycloalkyl, and heterocycloalkyl are optionally substituted with one to three R₅.

In some embodiments of the formulae above, Rₓ is H, n is 0, R₁ is (C₁-C₆)alkoxy, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, and R₃ is (C₆-C₁₀)aryl, (C₃-C₈)cycloalkyl, or 5- to 7-membered heterocycloalkyl comprising 1 to 3 heteroatoms selected from O, N, and S.

In some embodiments of the formulae above, Rₓ is H, n is 0, R₁ is (C₁-C₆)alkoxy, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, and R₃ is (C₆-C₁₀)aryl, 5- or 6-membered heteroaryl comprising 1 to 3 heteroatoms selected from O, N, and S, or (C₃-C₈)cycloalkyl, wherein the aryl, heteroaryl and cycloalkyl are optionally substituted with one to three R₅.

In some embodiments of the formulae above, Rₓ is H, n is 0, R₁ is (C₁-C₆)alkoxy, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, and R₃ is (C₆-C₁₀)aryl, 5- or 6-membered heteroaryl comprising 1 to 3 heteroatoms selected from O, N, and S, or (C₃-C₈)cycloalkyl.

In some embodiments of the formulae above, Rₓ is H, n is 0, R₁ is (C₁-C₆)alkoxy, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, and R₃ is (C₆-C₁₀)aryl optionally substituted with one to three R₅.

In some embodiments of the formulae above, Rₓ is H, n is 0, R₁ is (C₁-C₆)alkoxy, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, and R₃ is 5- or 6-membered heteroaryl comprising 1 to 3 heteroatoms selected from O, N, and S optionally substituted with one to three R₅.

In some embodiments of the formulae above, Rₓ is H, n is 0, R₁ is (C₁-C₆)alkoxy, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, and R₃ is (C₃-C₅)cycloalkyl optionally substituted with one to three R₅.

In some embodiments of the formulae above, Rₓ is H, n is 0, R₁ is (C₁-C₆)alkoxy, halogen, —OH, —(CH₂)₀₋₂NH₂, —(CH₂)₀₋₂NH(C₁-C₆)alkyl, —(CH₂)₀₋₂N((C₁-C₆)alkyl)₂, or CN, and R₃ is 5- to 7-membered heterocycloalkyl comprising 1 to 3 heteroatoms selected from O, N, and S, optionally substituted with one to three R₅.

In some embodiments of the formulae above, Rₓ is H, n is 0, and R₁ is (C₁-C₆)alkoxy, halogen, —OH, —(CH₂)₀₋₂NH₂, or CN. In another embodiment, Rₓ is H, n is 0, R₁ is (C₁-C₆)alkoxy, halogen, —OH, —(CH₂)₀₋₂NH₂, or CN, and R₃ is (C₁-C₆)alkyl optionally substituted with one to three R₄. In another embodiment, Rₓ is H, n is 0, R₁ is (C₁-C₆)alkoxy, halogen, —OH, —(CH₂)₀₋₂NH₂, or CN, and R₃ is (C₁-C₆)alkyl substituted with one to three R₄.

In some embodiments of the formulae above, Rₓ is H, n is 0, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is independently selected from —C(O)OR₆, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In another embodiment, Rₓ is H, n is 0, R₁ is (C₁-C₆)alkoxy, halogen, —OH, —(CH₂)₀₋₂NH₂, or CN, and R₃ is (C₁-C₆)alkyl substituted with one to three R₄, and each R₄ is independently selected from —C(O)OR₆, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In some embodiments of the formulae above, Rₓ is H, n is 0, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is independently selected from (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇. In another embodiment, Rₓ is H, n is 0, R₁ is (C₁-C₆)alkoxy, halogen, —OH, —(CH₂)₀₋₂NH₂, or CN, and R₃ is (C₁-C₆)alkyl substituted with one to three R₄, and each R₄ is independently selected from (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In some embodiments of the formulae above, Rₓ is H, n is 0, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is independently selected from halogen, —OH, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In another embodiment, Rₓ is H, n is 0, R₁ is (C₁-C₆)alkoxy, halogen, —OH, —(CH₂)₀₋₂NH₂, or CN, and R₃ is (C₁-C₆)alkyl substituted with one to three R₄, and each R₄ is independently selected from halogen, —OH, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In some embodiments of the formulae above, Rₓ is H, n is 0, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is independently selected from halogen, —OH, phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇.

In another embodiment, Rₓ is H, n is 0, R₁ is (C₁-C₆)alkoxy, halogen, —OH, —(CH₂)₀₋₂NH₂, or CN, and R₃ is (C₁-C₆)alkyl substituted with one to three R₄, and each R₄ is independently selected from halogen, —OH, phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇.

In some embodiments of the formulae above, Rₓ is H, n is 0, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is independently selected from phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇. In another embodiment, Rₓ is H, n is 0, R₁ is (C₁-C₆)alkoxy, halogen, —OH, —(CH₂)₀₋₂NH₂, or CN, and R₃ is (C₁-C₆)alkyl substituted with one to three R₄, and each R₄ is independently selected from phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇.

In some embodiments of the formulae above, Rₓ is H, n is 0, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is independently selected from phenyl and 5- or 6-membered heteroaryl comprising 1 to 3 heteroatoms selected from O, N, and S, wherein the phenyl and heteroaryl groups are optionally substituted with one to three R₇. In another embodiment, Rₓ is H, n is 0, R₁ is (C₁-C₆)alkoxy, halogen, —OH, —(CH₂)₀₋₂NH₂, or CN, and R₃ is (C₁-C₆)alkyl substituted with one to three R₄, and each R₄ is independently selected from phenyl and 5- or 6-membered heteroaryl comprising 1 to 3 heteroatoms selected from O, N, and S, wherein the phenyl and heteroaryl groups are optionally substituted with one to three R₇.

In some embodiments of the formulae above, Rₓ is H, n is 0, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is phenyl optionally substituted with one to three R₇. In another embodiment, Rₓ is H, n is 0, R₁ is (C₁-C₆)alkoxy, halogen, —OH, —(CH₂)₀₋₂NH₂, or CN, and R₃ is (C₁-C₆)alkyl substituted with one to three R₄, and each R₄ is phenyl optionally substituted with one to three R₇.

In some embodiments of the formulae above, Rₓ is H, n is 0, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is 5- or 6-membered heteroaryl comprising 1 to 3 heteroatoms selected from O, N, and S, optionally substituted with one to three R₇. In another embodiment, Rₓ is H, n is 0, R₁ is (C₁-C₆)alkoxy, halogen, —OH, —(CH₂)₀₋₂NH₂, or CN, and R₃ is (C₁-C₆)alkyl substituted with one to three R₄, and each R₄ is 5- or 6-membered heteroaryl comprising 1 to 3 heteroatoms selected from O, N, and S, optionally substituted with one to three R₇.

In some embodiments of the formulae above, Rₓ is H, n is 0, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is (C₃-C₈)cycloalkyl optionally substituted with one to three R₇. In another embodiment, Rₓ is H, n is 0, R₁ is (C₁-C₆)alkoxy, halogen, —OH, —(CH₂)₀₋₂NH₂, or CN, and R₃ is (C₁-C₆)alkyl substituted with one to three R₄, and each R₄ is (C₃-C₈)cycloalkyl optionally substituted with one to three R₇.

In some embodiments of the formulae above, Rₓ is H, n is 0, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is 5- to 7-membered heterocycloalkyl comprising 1 to 3 heteroatoms selected from O, N, and S, optionally substituted with one to three R₇. In another embodiment, Rₓ is H, n is 0, R₁ is (C₁-C₆)alkoxy, halogen, —OH, —(CH₂)₀₋₂NH₂, or CN, and R₃ is (C₁-C₆)alkyl substituted with one to three R₄, and each R₄ is 5- to 7-membered heterocycloalkyl comprising 1 to 3 heteroatoms selected from O, N, and S, optionally substituted with one to three R₇.

In some embodiments of the formulae above, Rₓ is H, n is 0, R₁ is (C₁-C₆)alkoxy, halogen, —OH, —(CH₂)₀₋₂NH₂, or CN, and R₃ is (C₆-C₁₀)aryl, (C₃-C₈)cycloalkyl, or 5- to 7-membered heterocycloalkyl comprising 1 to 3 heteroatoms selected from O, N, and S, wherein the aryl, cycloalkyl, and heterocycloalkyl are optionally substituted with one to three R₅.

In some embodiments of the formulae above, Rₓ is H, n is 0, R₁ is (C₁-C₆)alkoxy, halogen, —OH, —(CH₂)₀₋₂NH₂, or CN, and R₃ is (C₆-C₁₀)aryl, (C₃-C₈)cycloalkyl, or 5- to 7-membered heterocycloalkyl comprising 1 to 3 heteroatoms selected from O, N, and S.

In some embodiments of the formulae above, Rₓ is H, n is 0, R₁ is (C₁-C₆)alkoxy, halogen, —OH, —(CH₂)₀₋₂NH₂, or CN, and R₃ is (C₆-C₁₀)aryl, 5- or 6-membered heteroaryl comprising 1 to 3 heteroatoms selected from O, N, and S, or (C₃-C₈)cycloalkyl, wherein the aryl, heteroaryl and cycloalkyl are optionally substituted with one to three R₅.

In some embodiments of the formulae above, Rₓ is H, n is 0, R₁ is (C₁-C₆)alkoxy, halogen, —OH, —(CH₂)₀₋₂NH₂, or CN, and R₃ is (C₆-C₁₀)aryl, 5- or 6-membered heteroaryl comprising 1 to 3 heteroatoms selected from O, N, and S, or (C₃-C₈)cycloalkyl.

In some embodiments of the formulae above, Rₓ is H, n is 0, R₁ is (C₁-C₆)alkoxy, halogen, —OH, —(CH₂)₀₋₂NH₂, or CN, and R₃ is (C₆-C₁₀)aryl optionally substituted with one to three R₅.

In some embodiments of the formulae above, Rₓ is H, n is 0, R₁ is (C₁-C₆)alkoxy, halogen, —OH, —(CH₂)₀₋₂NH₂, or CN, and R₃ is 5- or 6-membered heteroaryl comprising 1 to 3 heteroatoms selected from O, N, and S optionally substituted with one to three R₅.

In some embodiments of the formulae above, Rₓ is H, n is 0, R₁ is (C₁-C₆)alkoxy, halogen, —OH, —(CH₂)₀₋₂NH₂, or CN, and R₃ is (C₃-C₈)cycloalkyl optionally substituted with one to three R₅.

In some embodiments of the formulae above, Rₓ is H, n is 0, R₁ is (C₁-C₆)alkoxy, halogen, —OH, —(CH₂)₀₋₂NH₂, or CN, and R₃ is 5- to 7-membered heterocycloalkyl comprising 1 to 3 heteroatoms selected from O, N, and S, optionally substituted with one to three R₅.

In some embodiments of the formulae above, Rₓ is H, n is 1, and each R₂ is independently (C₁-C₆)alkyl. In another embodiment, Rₓ is H, n is 1, each R₂ is independently (C₁-C₆)alkyl, and R₁ is (C₁-C₆)alkoxy, halogen, —OH, —(CH₂)₀₋₂NH₂, or CN. In another embodiment, Rₓ is H, n is 1, each R₂ is independently (C₁-C₆)alkyl, R₁ is (C₁-C₆)alkoxy, halogen, —OH, —(CH₂)₀₋₂NH₂, or CN, and R₃ is (C₁-C₆)alkyl optionally substituted with one to three R₄. In another embodiment, Rₓ is H,
n is 1, each R₂ is independently (C₁-C₆)alkyl, R₁ is (C₁-C₆)alkoxy, halogen, —OH, —(CH₂)₀₋₂NH₂, or CN, and R₃ is (C₁-C₆)alkyl substituted with one to three R₄.

In some embodiments of the formulae above, Rₓ is H, n is 1, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is independently selected from —C(O)OR₆, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇. In another embodiment, Rₓ is H, n is 1, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl substituted with one to three R₄, and each R₄ is independently selected from —C(O)OR₆, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In some embodiments of the formulae above, Rₓ is H, n is 1, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is independently selected from (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇. In another embodiment, Rₓ is H, n is 1, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl substituted with one to three R₄, and each R₄ is independently selected from (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In some embodiments of the formulae above, Rₓ is H, n is 1, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is independently selected from halogen, —OH, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇. In another embodiment, Rₓ is H, n is 1, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl substituted with one to three R₄, and each R₄ is independently selected from halogen, —OH, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In some embodiments of the formulae above, Rₓ is H, n is 1, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is independently selected from halogen, —OH, phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇. In another embodiment, Rₓ is H, n is 1, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl substituted with one to three R₄, and each R₄ is independently selected from halogen, —OH, phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇.

In some embodiments of the formulae above, Rₓ is H, n is 1, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is independently selected from phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇. In another embodiment, Rₓ is H, n is 1, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl substituted with one to three R₄, and each R₄ is independently selected from phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇.

In some embodiments of the formulae above, Rₓ is H, n is 1, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is independently selected from phenyl and 5- or 6-membered heteroaryl comprising 1 to 3 heteroatoms selected from O, N, and S, wherein the phenyl and heteroaryl groups are optionally substituted with one to three R₇. In another embodiment, Rₓ is H, n is 1, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl substituted with one to three R₄, and each R₄ is independently selected from phenyl and 5- or 6-membered heteroaryl comprising 1 to 3 heteroatoms selected from O, N, and S, wherein the phenyl and heteroaryl groups are optionally substituted with one to three R₇.

In some embodiments of the formulae above, Rₓ is H, n is 1, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is phenyl optionally substituted with one to three R₇.

In another embodiment, Rₓ is H, n is 1, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl substituted with one to three R₄, and each R₄ is phenyl optionally substituted with one to three R₇.

In some embodiments of the formulae above, Rₓ is H, n is 1, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is 5- or 6-membered heteroaryl comprising 1 to 3 heteroatoms selected from O, N, and S, optionally substituted with one to three R₇. In another embodiment, Rₓ is H, n is 1, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl substituted with one to three R₄, and each R₄ is 5- or 6-membered heteroaryl comprising 1 to 3 heteroatoms selected from O, N, and S, optionally substituted with one to three R₇.

In some embodiments of the formulae above, Rₓ is H, n is 1, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is (C₃-C₈)cycloalkyl optionally substituted with one to three R₇. In another embodiment, Rₓ is H, n is 1, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl substituted with one to three R₄, and each R₄ is (C₃-C₈)cycloalkyl optionally substituted with one to three R₇.

In some embodiments of the formulae above, Rₓ is H, n is 1, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is 5- to 7-membered heterocycloalkyl comprising 1 to 3 heteroatoms selected from O, N, and S, optionally substituted with one to three R₇. In another embodiment, Rₓ is H, n is 1, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl substituted with one to three R₄, and each R₄ is 5- to 7-membered heterocycloalkyl comprising 1 to 3 heteroatoms selected from O, N, and S, optionally substituted with one to three R₇.

In some embodiments of the formulae above, Rₓ is H, n is 1, each R₂ is independently (C₁-C₆)alkyl, R₁ is (C₁-C₆)alkoxy, halogen, —OH, —(CH₂)₀₋₂NH₂, or CN, and R₃ is (C₆-C₁₀)aryl, (C₃-C₈)cycloalkyl, or 5- to 7-membered heterocycloalkyl comprising 1 to 3 heteroatoms selected from O, N, and S, wherein the aryl, cycloalkyl, and heterocycloalkyl are optionally substituted with one to three R₅.

In some embodiments of the formulae above, Rₓ is H, n is 1, each R₂ is independently (C₁-C₆)alkyl, R₁ is (C₁-C₆)alkoxy, halogen, —OH, —(CH₂)₀₋₂NH₂, or CN, and R₃ is (C₆-C₁₀)aryl, (C₃-C₈)cycloalkyl, or 5- to 7-membered heterocycloalkyl comprising 1 to 3 heteroatoms selected from O, N, and S.

In some embodiments of the formulae above, Rₓ is H, n is 1, each R₂ is independently (C₁-C₆)alkyl, R₁ is (C₁-C₆)alkoxy, halogen, —OH, —(CH₂)₀₋₂NH₂, or CN, and R₃ is (C₆-C₁₀)aryl, 5- or 6-membered heteroaryl comprising 1 to 3 heteroatoms selected from O, N, and S, or (C₃-C₈)cycloalkyl, wherein the aryl, heteroaryl and cycloalkyl are optionally substituted with one to three R₅.

In some embodiments of the formulae above, Rₓ is H, n is 1, each R₂ is independently (C₁-C₆)alkyl, R₁ is (C₁-C₆)alkoxy, halogen, —OH, —(CH₂)₀₋₂NH₂, or CN, and R₃ is (C₆-C₁₀)aryl, 5- or 6-membered heteroaryl comprising 1 to 3 heteroatoms selected from O, N, and S, or (C₃-C₈)cycloalkyl.

In some embodiments of the formulae above, Rₓ is H, n is 1, each R₂ is independently (C₁-C₆)alkyl, R₁ is (C₁-C₆)alkoxy, halogen, —OH, —(CH₂)₀₋₂NH₂, or CN, and R₃ is (C₆-C₁₀)aryl optionally substituted with one to three R₅.

In some embodiments of the formulae above, Rₓ is H, n is 1, each R₂ is independently (C₁-C₆)alkyl, R₁ is (C₁-C₆)alkoxy, halogen, —OH, —(CH₂)₀₋₂NH₂, or CN, and R₃ is 5- or 6-membered heteroaryl comprising 1 to 3 heteroatoms selected from O, N, and S optionally substituted with one to three R₅.

In some embodiments of the formulae above, Rₓ is H, n is 1, each R₂ is independently (C₁-C₆)alkyl, R₁ is (C₁-C₆)alkoxy, halogen, —OH, —(CH₂)₀₋₂NH₂, or CN, and R₃ is (C₃-C₈)cycloalkyl optionally substituted with one to three R₅.

In some embodiments of the formulae above, Rₓ is H, n is 1, each R₂ is independently (C₁-C₆)alkyl, R₁ is (C₁-C₆)alkoxy, halogen, —OH, —(CH₂)₀₋₂NH₂, or CN, and R₃ is 5- to 7-membered heterocycloalkyl comprising 1 to 3 heteroatoms selected from O, N, and S, optionally substituted with one to three R₅.

In some embodiments of the formulae above, Rₓ is H, n is 0 or 1, and each R₂ is independently (C₁-C₆)alkyl. In another embodiment, Rₓ is H, n is 0 or 1, each R₂ is independently (C₁-C₆)alkyl, and R₁ is (C₁-C₆)alkoxy, halogen, —OH, —(CH₂)₀₋₂NH₂, or CN. In another embodiment, Rₓ is H, n is 0 or 1, each R₂ is independently (C₁-C₆)alkyl, R₁ is (C₁-C₆)alkoxy, halogen, —OH, —(CH₂)₀₋₂NH₂, or CN, and R₃ is (C₁-C₆)alkyl optionally substituted with one to three R₄. In another embodiment, Rₓ is H, n is 0 or 1, each R₂ is independently (C₁-C₆)alkyl, R₁ is (C₁-C₆)alkoxy, halogen, —OH, —(CH₂)₀₋₂NH₂, or CN, and R₃ is (C₁-C₆)alkyl substituted with one to three R₄.

In some embodiments of the formulae above, Rₓ is H, n is 0 or 1, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is independently selected from —C(O)OR₆, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇. In another embodiment, Rₓ is H, n is 0 or 1, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl substituted with one to three R₄, and each R₄ is independently selected from —C(O)OR₆, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In some embodiments of the formulae above, Rₓ is H, n is 0 or 1, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is independently selected from (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇. In another embodiment, Rₓ is H, n is 0 or 1, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl substituted with one to three R₄, and each R₄ is independently selected from (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In some embodiments of the formulae above, Rₓ is H, n is 0 or 1, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is independently selected from halogen, —OH, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇. In another embodiment, Rₓ is H, n is 0 or 1, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl substituted with one to three R₄, and each R₄ is independently selected from halogen, —OH, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In some embodiments of the formulae above, Rₓ is H, n is 0 or 1, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is independently selected from halogen, —OH, phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇. In another embodiment, Rₓ is H, n is 0 or 1, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl substituted with one to three R₄, and each R₄ is independently selected from halogen, —OH, phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇.

In some embodiments of the formulae above, Rₓ is H, n is 0 or 1, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is independently selected from phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇. In another embodiment, Rₓ is H, n is 0 or 1, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl substituted with one to three R₄, and each R₄ is independently selected from phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇.

In some embodiments of the formulae above, Rₓ is H, n is 0 or 1, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is independently selected from phenyl and 5- or 6-membered heteroaryl comprising 1 to 3 heteroatoms selected from O, N, and S, wherein the phenyl and heteroaryl groups are optionally substituted with one to three R₇. In another embodiment, Rₓ is H, n is 0 or 1, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl substituted with one to three R₄, and each R₄ is independently selected from phenyl and 5- or 6-membered heteroaryl comprising 1 to 3 heteroatoms selected from O, N, and S, wherein the phenyl and heteroaryl groups are optionally substituted with one to three R₇.

In some embodiments of the formulae above, Rₓ is H, n is 0 or 1, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is phenyl optionally substituted with one to three R₇. In another embodiment, Rₓ is H, n is 0 or 1, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl substituted with one to three R₄, and each R₄ is phenyl optionally substituted with one to three R₇.

In some embodiments of the formulae above, Rₓ is H, n is 0 or 1, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is 5- or 6-membered heteroaryl comprising 1 to 3 heteroatoms selected from O, N, and S, optionally substituted with one to three R₇. In another embodiment, Rₓ is H, n is 0 or 1, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl substituted with one to three R₄, and each R₄ is 5- or 6-membered heteroaryl comprising 1 to 3 heteroatoms selected from O, N, and S, optionally substituted with one to three R₇.

In some embodiments of the formulae above, Rₓ is H, n is 0 or 1, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is (C₃-C₈)cycloalkyl optionally substituted with one to three R₇. In another embodiment, Rₓ is H, n is 0 or 1, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl substituted with one to three R₄, and each R₄ is (C₃-C₈)cycloalkyl optionally substituted with one to three R₇.

In some embodiments of the formulae above, Rₓ is H, n is 0 or 1, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is 5- to 7-membered heterocycloalkyl comprising 1 to 3 heteroatoms selected from O, N, and S, optionally substituted with one to three R₇. In another embodiment, Rₓ is H, n is 0 or 1, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl substituted with one to three R₄, and each R₄ is 5- to 7-membered heterocycloalkyl comprising 1 to 3 heteroatoms selected from O, N, and S, optionally substituted with one to three R₇.

In some embodiments of the formulae above, Rₓ is H, n is 0 or 1, each R₂ is independently (C₁-C₆)alkyl, R₁ is (C₁-C₆)alkoxy, halogen, —OH, —(CH₂)₀₋₂NH₂, or CN, and R₃ is (C₆-C₁₀)aryl, (C₃-C₈)cycloalkyl, or 5- to 7-membered heterocycloalkyl comprising 1 to 3 heteroatoms selected from O, N, and S, wherein the aryl, cycloalkyl, and heterocycloalkyl are optionally substituted with one to three R₅.

In some embodiments of the formulae above, Rₓ is H, n is 0 or 1, each R₂ is independently (C₁-C₆)alkyl, R₁ is (C₁-C₆)alkoxy, halogen, —OH, —(CH₂)₀₋₂NH₂, or CN, and R₃ is (C₆-C₁₀)aryl, (C₃-C₈)cycloalkyl, or 5- to 7-membered heterocycloalkyl comprising 1 to 3 heteroatoms selected from O, N, and S.

In some embodiments of the formulae above, Rₓ is H, n is 0 or 1, each R₂ is independently (C₁-C₆)alkyl, R₁ is (C₁-C₆)alkoxy, halogen, —OH, —(CH₂)₀₋₂NH₂, or CN, and R₃ is (C₆-C₁₀)aryl, 5- or 6-membered heteroaryl comprising 1 to 3 heteroatoms selected from O, N, and S, or (C₃-C₈)cycloalkyl, wherein the aryl, heteroaryl and cycloalkyl are optionally substituted with one to three R₅.

In some embodiments of the formulae above, Rₓ is H, n is 0 or 1, each R₂ is independently (C₁-C₆)alkyl, R₁ is (C₁-C₆)alkoxy, halogen, —OH, —(CH₂)₀₋₂NH₂, or CN, and R₃ is (C₆-C₁₀)aryl, 5- or 6-membered heteroaryl comprising 1 to 3 heteroatoms selected from O, N, and S, or (C₃-C₈)cycloalkyl.

In some embodiments of the formulae above, Rₓ is H, n is 0 or 1, each R₂ is independently (C₁-C₆)alkyl, R₁ is (C₁-C₆)alkoxy, halogen, —OH, —(CH₂)₀₋₂NH₂, or CN, and R₃ is (C₆-C₁₀)aryl optionally substituted with one to three R₅.

In some embodiments of the formulae above, Rₓ is H, n is 0 or 1, each R₂ is independently (C₁-C₆)alkyl, R₁ is (C₁-C₆)alkoxy, halogen, —OH, —(CH₂)₀₋₂NH₂, or CN, and R₃ is 5- or 6-membered heteroaryl comprising 1 to 3 heteroatoms selected from O, N, and S optionally substituted with one to three R₅.

In some embodiments of the formulae above, Rₓ is H, n is 0 or 1, each R₂ is independently (C₁-C₆)alkyl, R₁ is (C₁-C₆)alkoxy, halogen, —OH, —(CH₂)₀₋₂NH₂, or CN, and R₃ is (C₃-C₈)cycloalkyl optionally substituted with one to three R₅.

In some embodiments of the formulae above, Rₓ is H, n is 0 or 1, each R₂ is independently (C₁-C₆)alkyl, R₁ is (C₁-C₆)alkoxy, halogen, —OH, —(CH₂)₀₋₂NH₂, or CN, and R₃ is 5- to 7-membered heterocycloalkyl comprising 1 to 3 heteroatoms selected from O, N, and S, optionally substituted with one to three R₅.

In some embodiments of the formulae above, Rₓ is H, n is 1 or 2, and each R₂ is independently (C₁-C₆)alkyl. In another embodiment, Rₓ is H, n is 1, each R₂ is independently (C₁-C₆)alkyl, and R₁ is (C₁-C₆)alkoxy, halogen, —OH, —(CH₂)₀₋₂NH₂, or CN. In another embodiment, Rₓ is H, n is 1 or 2, each R₂ is independently (C₁-C₆)alkyl, R₁ is (C₁-C₆)alkoxy, halogen, —OH, —(CH₂)₀₋₂NH₂, or CN, and R₃ is (C₁-C₆)alkyl optionally substituted with one to three R₄. In another embodiment, Rₓ is H, n is 1 or 2, each R₂ is independently (C₁-C₆)alkyl, R₁ is (C₁-C₆)alkoxy, halogen, —OH, —(CH₂)₀₋₂NH₂, or CN, and R₃ is (C₁-C₆)alkyl substituted with one to three R₄.

In some embodiments of the formulae above, Rₓ is H, n is 1 or 2, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is independently selected from —C(O)OR₆, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇. In another embodiment, Rₓ is H, n is 1 or 2, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl substituted with one to three R₄, and each R₄ is independently selected from —C(O)OR₆, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In some embodiments of the formulae above, Rₓ is H, n is 1 or 2, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is independently selected from (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇. In another embodiment, Rₓ is H, n is 1 or 2, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl substituted with one to three R₄, and each R₄ is independently selected from (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In some embodiments of the formulae above, Rₓ is H, n is 1 or 2, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is independently selected from halogen, —OH, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇. In another embodiment, Rₓ is H, n is 1 or 2, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl substituted with one to three R₄, and each R₄ is independently selected from halogen, —OH, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In some embodiments of the formulae above, Rₓ is H, n is 1 or 2, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is independently selected from halogen, —OH, phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇. In another embodiment, Rₓ is H, n is 1 or 2, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl substituted with one to three R₄, and each R₄ is independently selected from halogen, —OH, phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇.

In some embodiments of the formulae above, Rₓ is H, n is 1 or 2, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is independently selected from phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇. In another embodiment, Rₓ is H, n is 1 or 2, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl substituted with one to three R₄, and each R₄ is independently selected from phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇.

In some embodiments of the formulae above, Rₓ is H, n is 1 or 2, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is independently selected from phenyl and 5- or 6-membered heteroaryl comprising 1 to 3 heteroatoms selected from O, N, and S, wherein the phenyl and heteroaryl groups are optionally substituted with one to three R₇. In another embodiment, Rₓ is H, n is 1 or 2, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl substituted with one to three R₄, and each R₄ is independently selected from phenyl and 5- or 6-membered heteroaryl comprising 1 to 3 heteroatoms selected from O, N, and S, wherein the phenyl and heteroaryl groups are optionally substituted with one to three R₇.

In some embodiments of the formulae above, Rₓ is H, n is 1 or 2, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is phenyl optionally substituted with one to three R₇. In another embodiment, Rₓ is H, n is 1 or 2, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl substituted with one to three R₄, and each R₄ is phenyl optionally substituted with one to three R₇.

In some embodiments of the formulae above, Rₓ is H, n is 1 or 2, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is 5- or 6-membered heteroaryl comprising 1 to 3 heteroatoms selected from O, N, and S, optionally substituted with one to three R₇. In another embodiment, Rₓ is H, n is 1 or 2, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl substituted with one to three R₄, and each R₄ is 5- or 6-membered heteroaryl comprising 1 to 3 heteroatoms selected from O, N, and S, optionally substituted with one to three R₇.

In some embodiments of the formulae above, Rₓ is H, n is 1 or 2, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is (C₃-C₈)cycloalkyl optionally substituted with one to three R₇. In another embodiment, Rₓ is H, n is 1 or 2, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl substituted with one to three R₄, and each R₄ is (C₃-C₈)cycloalkyl optionally substituted with one to three R₇.

In some embodiments of the formulae above, Rₓ is H, n is 1 or 2, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is 5- to 7-membered heterocycloalkyl comprising 1 to 3 heteroatoms selected from O, N, and S, optionally substituted with one to three R₇. In another embodiment, Rₓ is H, n is 1 or 2, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl substituted with one to three R₄, and each R₄ is 5- to 7-membered heterocycloalkyl comprising 1 to 3 heteroatoms selected from O, N, and S, optionally substituted with one to three R₇.

In some embodiments of the formulae above, Rₓ is H, n is 1 or 2, each R₂ is independently (C₁-C₆)alkyl, R₁ is (C₁-C₆)alkoxy, halogen, —OH, —(CH₂)₀₋₂NH₂, or CN, and R₃ is (C₆-C₁₀)aryl, (C₃-C₅)cycloalkyl, or 5- to 7-membered heterocycloalkyl comprising 1 to 3 heteroatoms selected from O, N, and S, wherein the aryl, cycloalkyl, and heterocycloalkyl are optionally substituted with one to three R₅.

In some embodiments of the formulae above, Rₓ is H, n is 1 or 2, each R₂ is independently (C₁-C₆)alkyl, R₁ is (C₁-C₆)alkoxy, halogen, —OH, —(CH₂)₀₋₂NH₂, or CN, and R₃ is (C₆-C₁₀)aryl, (C₃-C₅)cycloalkyl, or 5- to 7-membered heterocycloalkyl comprising 1 to 3 heteroatoms selected from O, N, and S.

In some embodiments of the formulae above, Rₓ is H, n is 1 or 2, each R₂ is independently (C₁-C₆)alkyl, R₁ is (C₁-C₆)alkoxy, halogen, —OH, —(CH₂)₀₋₂NH₂, or CN, and R₃ is (C₆-C₁₀)aryl, 5- or 6-membered heteroaryl comprising 1 to 3 heteroatoms selected from O, N, and S, or (C₃-C₅)cycloalkyl, wherein the aryl, heteroaryl and cycloalkyl are optionally substituted with one to three R₅.

In some embodiments of the formulae above, Rₓ is H, n is 1 or 2, each R₂ is independently (C₁-C₆)alkyl, R₁ is (C₁-C₆)alkoxy, halogen, —OH, —(CH₂)₀₋₂NH₂, or CN, and R₃ is (C₆-C₁₀)aryl, 5- or 6-membered heteroaryl comprising 1 to 3 heteroatoms selected from O, N, and S, or (C₃-C₅)cycloalkyl.

In some embodiments of the formulae above, Rₓ is H, n is 1 or 2, each R₂ is independently (C₁-C₆)alkyl, R₁ is (C₁-C₆)alkoxy, halogen, —OH, —(CH₂)₀₋₂NH₂, or CN, and R₃ is (C₆-C₁₀)aryl optionally substituted with one to three R₅.

In some embodiments of the formulae above, Rₓ is H, n is 1 or 2, each R₂ is independently (C₁-C₆)alkyl, R₁ is (C₁-C₆)alkoxy, halogen, —OH, —(CH₂)₀₋₂NH₂, or CN, and R₃ is 5- or 6-membered heteroaryl comprising 1 to 3 heteroatoms selected from O, N, and S optionally substituted with one to three R₅.

In some embodiments of the formulae above, Rₓ is H, n is 1 or 2, each R₂ is independently (C₁-C₆)alkyl, R₁ is (C₁-C₆)alkoxy, halogen, —OH, —(CH₂)₀₋₂NH₂, or CN, and R₃ is (C₃-C₈)cycloalkyl optionally substituted with one to three R₅.

In some embodiments of the formulae above, Rₓ is H, n is 1 or 2, each R₂ is independently (C₁-C₆)alkyl, R₁ is (C₁-C₆)alkoxy, halogen, —OH, —(CH₂)₀₋₂NH₂, or CN, and R₃ is 5- to 7-membered heterocycloalkyl comprising 1 to 3 heteroatoms selected from O, N, and S, optionally substituted with one to three R₅.

In some embodiments of the formulae above, Rₓ is H, n is 2, and each R₂ is independently (C₁-C₆)alkyl. In another embodiment, Rₓ is H, n is 2, each R₂ is independently (C₁-C₆)alkyl, and R₁ is (C₁-C₆)alkoxy, halogen, —OH, —(CH₂)₀₋₂NH₂, or CN. In another embodiment, Rₓ is H, n is 2, each R₂ is independently (C₁-C₆)alkyl, R₁ is (C₁-C₆)alkoxy, halogen, —OH, —(CH₂)₀₋₂NH₂, or CN, and R₃ is (C₁-C₆)alkyl optionally substituted with one to three R₄. In another embodiment, Rₓ is H, n is 2, each R₂ is independently (C₁-C₆)alkyl, R₁ is (C₁-C₆)alkoxy, halogen, —OH, —(CH₂)₀₋₂NH₂, or CN, and R₃ is (C₁-C₆)alkyl substituted with one to three R₄.

In some embodiments of the formulae above, Rₓ is H, n is 2, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is independently selected from —C(O)OR₆, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇. In another embodiment, Rₓ is H, n is 2, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl substituted with one to three R₄, and each R₄ is independently selected from —C(O)OR₆, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In some embodiments of the formulae above, Rₓ is H, n is 2, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is independently selected from (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇. In another embodiment, Rₓ is H, n is 2, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl substituted with one to three R₄, and each R₄ is independently selected from (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In some embodiments of the formulae above, Rₓ is H, n is 2, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is independently selected from halogen, —OH, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇. In another embodiment, Rₓ is H, n is 2, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl substituted with one to three R₄, and each R₄ is independently selected from halogen, —OH, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In some embodiments of the formulae above, Rₓ is H, n is 2, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is independently selected from halogen, —OH, phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇. In another embodiment, Rₓ is H, n is 2, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl substituted with one to three R₄, and each R₄ is independently selected from halogen, —OH, phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇.

In some embodiments of the formulae above, Rₓ is H, n is 2, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is independently selected from phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇. In another embodiment, Rₓ is H, n is 2, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl substituted with one to three R₄, and each R₄ is independently selected from phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇.

In some embodiments of the formulae above, Rₓ is H, n is 2, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is independently selected from phenyl and 5- or 6-membered heteroaryl comprising 1 to 3 heteroatoms selected from O, N, and S, wherein the aryl and heteroaryl groups are optionally substituted with one to three R₇. In another embodiment, Rₓ is H, n is 2, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl substituted with one to three R₄, and each R₄ is independently selected from phenyl and 5- or 6-membered heteroaryl comprising 1 to 3 heteroatoms selected from O, N, and S, wherein the aryl and heteroaryl groups are optionally substituted with one to three R₇.

In some embodiments of the formulae above, Rₓ is H, n is 2, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is phenyl optionally substituted with one to three R₇.

In another embodiment, Rₓ is H, n is 2, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl substituted with one to three R₄, and each R₄ is phenyl optionally substituted with one to three R₇.

In some embodiments of the formulae above, Rₓ is H, n is 2, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is 5- or 6-membered heteroaryl comprising 1 to 3 heteroatoms selected from O, N, and S, optionally substituted with one to three R₇. In another embodiment, Rₓ is H, n is 2, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl substituted with one to three R₄, and each R₄ is 5- or 6-membered heteroaryl comprising 1 to 3 heteroatoms selected from O, N, and S, optionally substituted with one to three R₇.

In some embodiments of the formulae above, Rₓ is H, n is 2, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is (C₃-C₈)cycloalkyl optionally substituted with one to three R₇. In another embodiment, Rₓ is H, n is 2, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl substituted with one to three R₄, and each R₄ is (C₃-C₈)cycloalkyl optionally substituted with one to three R₇.

In some embodiments of the formulae above, Rₓ is H, n is 2, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is 5- to 7-membered heterocycloalkyl comprising 1 to 3 heteroatoms selected from O, N, and S, optionally substituted with one to three R₇. In another embodiment, Rₓ is H, n is 2, each R₂ is independently (C₁-C₆)alkyl, R₁ is (C₁-C₆)alkoxy, halogen, —OH, —(CH₂)₀₋₂NH₂, or CN, R₃ is (C₁-C₆)alkyl substituted with one to three R₄, and each R₄ is 5- to 7-membered heterocycloalkyl comprising 1 to 3 heteroatoms selected from O, N, and S, optionally substituted with one to three R₇.

In some embodiments of the formulae above, Rₓ is H, n is 2, each R₂ is independently (C₁-C₆)alkyl, R₁ is (C₁-C₆)alkoxy, halogen, —OH, —(CH₂)₀₋₂NH₂, or CN, and R₃ is (C₆-C₁₀)aryl, (C₃-C₈)cycloalkyl, or 5- to 7-membered heterocycloalkyl comprising 1 to 3 heteroatoms selected from O, N, and S, wherein the aryl, cycloalkyl, and heterocycloalkyl are optionally substituted with one to three R₅.

In some embodiments of the formulae above, Rₓ is H, n is 2, each R₂ is independently (C₁-C₆)alkyl, R₁ is (C₁-C₆)alkoxy, halogen, —OH, —(CH₂)₀₋₂NH₂, or CN, and R₃ is (C₆-C₁₀)aryl, (C₃-C₈)cycloalkyl, or 5- to 7-membered heterocycloalkyl comprising 1 to 3 heteroatoms selected from O, N, and S.

In some embodiments of the formulae above, Rₓ is H, n is 2, each R₂ is independently (C₁-C₆)alkyl, R₁ is (C₁-C₆)alkoxy, halogen, —OH, —(CH₂)₀₋₂NH₂, or CN, and R₃ is (C₆-C₁₀)aryl, 5- or 6-membered heteroaryl comprising 1 to 3 heteroatoms selected from O, N, and S, or (C₃-C₈)cycloalkyl, wherein the aryl, heteroaryl and cycloalkyl are optionally substituted with one to three R₅.

In some embodiments of the formulae above, Rₓ is H, n is 2, each R₂ is independently (C₁-C₆)alkyl, R₁ is (C₁-C₆)alkoxy, halogen, —OH, —(CH₂)₀₋₂NH₂, or CN, and R₃ is (C₆-C₁₀)aryl, 5- or 6-membered heteroaryl comprising 1 to 3 heteroatoms selected from O, N, and S, or (C₃-C₈)cycloalkyl.

In some embodiments of the formulae above, Rₓ is H, n is 2, each R₂ is independently (C₁-C₆)alkyl, R₁ is (C₁-C₆)alkoxy, halogen, —OH, —(CH₂)₀₋₂NH₂, or CN, and R₃ is (C₆-C₁₀)aryl optionally substituted with one to three R₅.

In some embodiments of the formulae above, Rₓ is H, n is 2, each R₂ is independently (C₁-C₆)alkyl, R₁ is (C₁-C₆)alkoxy, halogen, —OH, —(CH₂)₀₋₂NH₂, or CN, and R₃ is 5- or 6-membered heteroaryl comprising 1 to 3 heteroatoms selected from O, N, and S optionally substituted with one to three R₅.

In some embodiments of the formulae above, Rₓ is H, n is 2, each R₂ is independently (C₁-C₆)alkyl, R₁ is (C₁-C₆)alkoxy, halogen, —OH, —(CH₂)₀₋₂NH₂, or CN, and R₃ is (C₃-C₈)cycloalkyl optionally substituted with one to three R₅.

In some embodiments of the formulae above, Rₓ is H, n is 2, each R₂ is independently (C₁-C₆)alkyl, R₁ is (C₁-C₆)alkoxy, halogen, —OH, —(CH₂)₀₋₂NH₂, or CN, and R₃ is 5- to 7-membered heterocycloalkyl comprising 1 to 3 heteroatoms selected from O, N, and S, optionally substituted with one to three R₅.

In some embodiments of the formulae above, Rₓ is H, n is 2, and each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S. In another embodiment, Rₓ is H, n is 2, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, and R₁ is (C₁-C₆)alkoxy, halogen, —OH, —(CH₂)₀₋₂NH₂, or CN. In another embodiment, Rₓ is H, n is 2, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, and R₃ is (C₁-C₆)alkyl optionally substituted with one to three R₄. In another embodiment, Rₓ is H, n is 2, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, and R₃ is (C₁-C₆)alkyl substituted with one to three R₄.

In some embodiments of the formulae above, Rₓ is H, n is 2, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is independently selected from —C(O)OR₆, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇. In another embodiment, Rₓ is H, n is 2, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, R₃ is (C₁-C₆)alkyl substituted with one to three R₄, and each R₄ is independently selected from —C(O)OR₆, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In some embodiments of the formulae above, Rₓ is H, n is 2, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is independently selected from (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇. In another embodiment, Rₓ is H, n is 2, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, R₃ is (C₁-C₆)alkyl substituted with one to three R₄, and each R₄ is independently selected from (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In some embodiments of the formulae above, Rₓ is H, n is 2, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is independently selected from halogen, —OH, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇. In another embodiment, Rₓ is H, n is 2, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, R₃ is (C₁-C₆)alkyl substituted with one to three R₄, and each R₄ is independently selected from halogen, —OH, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In some embodiments of the formulae above, Rₓ is H, n is 2, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is independently selected from halogen, —OH, phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇. In another embodiment, Rₓ is H, n is 2, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, R₃ is (C₁-C₆)alkyl substituted with one to three R₄, and each R₄ is independently selected from halogen, —OH, phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇.

In some embodiments of the formulae above, Rₓ is H, n is 2, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is independently selected from phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇. In another embodiment, Rₓ is H, n is 2, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, R₃ is (C₁-C₆)alkyl substituted with one to three R₄, and each R₄ is independently selected from phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇.

In some embodiments of the formulae above, Rₓ is H, n is 2, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is independently selected from phenyl and 5- or 6-membered heteroaryl comprising 1 to 3 heteroatoms selected from O, N, and S, wherein the phenyl and heteroaryl groups are optionally substituted with one to three R₇. In another embodiment, Rₓ is H, n is 2, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, R₃ is (C₁-C₆)alkyl substituted with one to three R₄, and each R₄ is independently selected from phenyl and 5- or 6-membered heteroaryl comprising 1 to 3 heteroatoms selected from O, N, and S, wherein the phenyl and heteroaryl groups are optionally substituted with one to three R₇.

In some embodiments of the formulae above, Rₓ is H, n is 2, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is phenyl optionally substituted with one to three R₇. In another embodiment, Rₓ is H, n is 2, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, R₃ is (C₁-C₆)alkyl substituted with one to three R₄, and each R₄ is phenyl optionally substituted with one to three R₇.

In some embodiments of the formulae above, Rₓ is H, n is 2, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is 5- or 6-membered heteroaryl comprising 1 to 3 heteroatoms selected from O, N, and S, optionally substituted with one to three R₇. In another embodiment, Rₓ is H, n is 2, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, R₃ is (C₁-C₆)alkyl substituted with one to three R₄, and each R₄ is 5- or 6-membered heteroaryl comprising 1 to 3 heteroatoms selected from O, N, and S, optionally substituted with one to three R₇.

In some embodiments of the formulae above, Rₓ is H, n is 2, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is (C₃-C₈)cycloalkyl optionally substituted with one to three R₇. In another embodiment, Rₓ is H, n is 2, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, R₃ is (C₁-C₆)alkyl substituted with one to three R₄, and each R₄ is (C₃-C₈)cycloalkyl optionally substituted with one to three R₇.

In some embodiments of the formulae above, Rₓ is H, n is 2, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is 5- to 7-membered heterocycloalkyl comprising 1 to 3 heteroatoms selected from O, N, and S, optionally substituted with one to three R₇. In another embodiment, Rₓ is H, n is 2, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, R₃ is (C₁-C₆)alkyl substituted with one to three R₄, and each R₄ is 5- to 7-membered heterocycloalkyl comprising 1 to 3 heteroatoms selected from O, N, and S, optionally substituted with one to three R₇.

In some embodiments of the formulae above, Rₓ is H, n is 2, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, and R₃ is (C₆-C₁₀)aryl, (C₃-C₈)cycloalkyl, or 5- to 7-membered heterocycloalkyl comprising 1 to 3 heteroatoms selected from O, N, and S, wherein the aryl, cycloalkyl, and heterocycloalkyl are optionally substituted with one to three R₅.

In some embodiments of the formulae above, Rₓ is H, n is 2, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, and R₃ is (C₆-C₁₀)aryl, (C₃-C₈)cycloalkyl, or 5- to 7-membered heterocycloalkyl comprising 1 to 3 heteroatoms selected from O, N, and S.

In some embodiments of the formulae above, Rₓ is H, n is 2, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, and R₃ is (C₆-C₁₀)aryl, 5- or 6-membered heteroaryl comprising 1 to 3 heteroatoms selected from O, N, and S, or (C₃-C₈)cycloalkyl, wherein the aryl, heteroaryl and cycloalkyl are optionally substituted with one to three R₅.

In some embodiments of the formulae above, Rₓ is H, n is 2, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, and R₃ is (C₆-C₁₀)aryl, 5- or 6-membered heteroaryl comprising 1 to 3 heteroatoms selected from O, N, and S, or (C₃-C₈)cycloalkyl.

In some embodiments of the formulae above, Rₓ is H, n is 2, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, and R₃ is (C₆-C₁₀)aryl optionally substituted with one to three R₅.

In some embodiments of the formulae above, Rₓ is H, n is 2, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, and R₃ is 5- or 6-membered heteroaryl comprising 1 to 3 heteroatoms selected from O, N, and S optionally substituted with one to three R₅.

In some embodiments of the formulae above, Rₓ is H, n is 2, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, and R₃ is (C₃-C₈)cycloalkyl optionally substituted with one to three R₅.

In some embodiments of the formulae above, Rₓ is H, n is 2, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, and R₃ is 5- to 7-membered heterocycloalkyl comprising 1 to 3 heteroatoms selected from O, N, and S, optionally substituted with one to three R₅.

In some embodiments of the formulae above, Rₓ is H, n is 3, and each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S. In another embodiment, Rₓ is H, n is 3, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, and R₁ is (C₁-C₆)alkoxy, halogen, —OH, —(CH₂)₀₋₂NH₂, or CN. In another embodiment, Rₓ is H, n is 3, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, and R₃ is (C₁-C₆)alkyl optionally substituted with one to three R₄. In another embodiment, Rₓ is H, n is 3, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, and R₃ is (C₁-C₆)alkyl substituted with one to three R₄.

In some embodiments of the formulae above, Rₓ is H, n is 3, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is independently selected from —C(O)OR₆, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇. In another embodiment, Rₓ is H, n is 3, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, R₃ is (C₁-C₆)alkyl substituted with one to three R₄, and each R₄ is independently selected from —C(O)OR₆, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In some embodiments of the formulae above, Rₓ is H, n is 3, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is independently selected from (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇. In another embodiment, Rₓ is H, n is 3, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, R₃ is (C₁-C₆)alkyl substituted with one to three R₄, and each R₄ is independently selected from (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In some embodiments of the formulae above, Rₓ is H, n is 3, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is independently selected from halogen, —OH, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇. In another embodiment, Rₓ is H, n is 3, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, R₃ is (C₁-C₆)alkyl substituted with one to three R₄, and each R₄ is independently selected from halogen, —OH, (C₆-C₁₀)aryl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the aryl, heteroaryl, cycloalkyl, and heterocycloalkyl groups are optionally substituted with one to three R₇.

In some embodiments of the formulae above, Rₓ is H, n is 3, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is independently selected from halogen, —OH, phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇. In another embodiment, Rₓ is H, n is 3, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, R₃ is (C₁-C₆)alkyl substituted with one to three R₄, and each R₄ is independently selected from halogen, —OH, phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇.

In some embodiments of the formulae above, Rₓ is H, n is 3, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is independently selected from phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇. In another embodiment, Rₓ is H, n is 3, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, R₃ is (C₁-C₆)alkyl substituted with one to three R₄, and each R₄ is independently selected from phenyl, 5- or 6-membered heteroaryl comprising 1 to 3 heteroatoms selected from O, N, and S, (C₃-C₈)cycloalkyl, and 5- to 7-membered heterocycloalkyl comprising 1 to 3 heteroatoms selected from O, N, and S, wherein the phenyl, heteroaryl, cycloalkyl, and heterocycloalkyl groups are optionally substituted with one to three R₇.

In some embodiments of the formulae above, Rₓ is H, n is 3, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is independently selected from phenyl and 5- or 6-membered heteroaryl comprising 1 to 3 heteroatoms selected from O, N, and S, wherein the phenyl and heteroaryl groups are optionally substituted with one to three R₇. In another embodiment, Rₓ is H, n is 3, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, R₃ is (C₁-C₆)alkyl substituted with one to three R₄, and each R₄ is independently selected from phenyl and 5- or 6-membered heteroaryl comprising 1 to 3 heteroatoms selected from O, N, and S, wherein the phenyl and heteroaryl groups are optionally substituted with one to three R₇.

In some embodiments of the formulae above, Rₓ is H, n is 3, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is phenyl optionally substituted with one to three R₇. In another embodiment, Rₓ is H, n is 3, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, R₃ is (C₁-C₆)alkyl substituted with one to three R₄, and each R₄ is phenyl optionally substituted with one to three R₇.

In some embodiments of the formulae above, Rₓ is H, n is 3, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is 5- or 6-membered heteroaryl comprising 1 to 3 heteroatoms selected from O, N, and S, optionally substituted with one to three R₇. In another embodiment, Rₓ is H, n is 3, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, R₃ is (C₁-C₆)alkyl substituted with one to three R₄, and each R₄ is 5- or 6-membered heteroaryl comprising 1 to 3 heteroatoms selected from O, N, and S, optionally substituted with one to three R₇.

In some embodiments of the formulae above, Rₓ is H, n is 3, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is (C₃-C₈)cycloalkyl optionally substituted with one to three R₇. In another embodiment, Rₓ is H, n is 3, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, R₃ is (C₁-C₆)alkyl substituted with one to three R₄, and each R₄ is (C₃-C₈)cycloalkyl optionally substituted with one to three R₇.

In some embodiments of the formulae above, Rₓ is H, n is 3, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, R₃ is (C₁-C₆)alkyl optionally substituted with one to three R₄, and each R₄ is 5- to 7-membered heterocycloalkyl comprising 1 to 3 heteroatoms selected from O, N, and S, optionally substituted with one to three R₇. In another embodiment, Rₓ is H, n is 3, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, R₃ is (C₁-C₆)alkyl substituted with one to three R₄, and each R₄ is 5- to 7-membered heterocycloalkyl comprising 1 to 3 heteroatoms selected from O, N, and S, optionally substituted with one to three R₇.

In some embodiments of the formulae above, Rₓ is H, n is 3, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, and R₃ is (C₆-C₁₀)aryl, (C₃-C₈)cycloalkyl, or 5- to 7-membered heterocycloalkyl comprising 1 to 3 heteroatoms selected from O, N, and S, wherein the aryl, cycloalkyl, and heterocycloalkyl are optionally substituted with one to three R₅.

In some embodiments of the formulae above, Rₓ is H, n is 3, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, and R₃ is (C₆-C₁₀)aryl, (C₃-C₈)cycloalkyl, or 5- to 7-membered heterocycloalkyl comprising 1 to 3 heteroatoms selected from O, N, and S.

In some embodiments of the formulae above, Rₓ is H, n is 3, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, and R₃ is (C₆-C₁₀)aryl, 5- or 6-membered heteroaryl comprising 1 to 3 heteroatoms selected from O, N, and S, or (C₃-C₈)cycloalkyl, wherein the aryl, heteroaryl and cycloalkyl are optionally substituted with one to three R₅.

In some embodiments of the formulae above, Rₓ is H, n is 3, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, and R₃ is (C₆-C₁₀)aryl, 5- or 6-membered heteroaryl comprising 1 to 3 heteroatoms selected from O, N, and S, or (C₃-C₈)cycloalkyl.

In some embodiments of the formulae above, Rₓ is H, n is 3, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, and R₃ is (C₆-C₁₀)aryl optionally substituted with one to three R₅.

In some embodiments of the formulae above, Rₓ is H, n is 3, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, and R₃ is 5- or 6-membered heteroaryl comprising 1 to 3 heteroatoms selected from O, N, and S optionally substituted with one to three R₅.

In some embodiments of the formulae above, Rₓ is H, n is 3, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, and R₃ is (C₃-C₈)cycloalkyl optionally substituted with one to three R₅.

In some embodiments of the formulae above, Rₓ is H, n is 3, each R₂ is independently (C₁-C₆)alkyl, or two R₂ together with the carbon atoms to which they are attached form (C₃-C₇)cycloalkyl or a 4- to 6-membered heterocycloalkyl ring comprising 1 to 3 heteroatoms selected from O, N, and S, R₁ is (C₁-C₆)alkoxy, halogen, —OH, —(CH₂)₀₋₂NH₂, or CN, and R₃ is 5- to 7-membered heterocycloalkyl comprising 1 to 3 heteroatoms selected from O, N, and S, optionally substituted with one to three R₅.

In some embodiments of the formulae above, R₃ is (C₁-C₆)alkyl optionally substituted with one to three R₄, and each R₄ is phenyl optionally substituted with one to three R₇.

In some embodiments of the formulae above, R₃ is (C₁-C₆)alkyl substituted with one to three R₄, and each R₄ is phenyl optionally substituted with one to three R₇.

In some embodiments of the formulae above, R₃ is (C₁-C₆)alkyl optionally substituted with one to three R₄, and each R₄ is 5- or 6-membered heteroaryl comprising 1 to 3 heteroatoms selected from O, N, and S, optionally substituted with one to three R₇.

In some embodiments of the formulae above, R₃ is (C₁-C₆)alkyl substituted with one to three R₄, and each R₄ is 5- or 6-membered heteroaryl comprising 1 to 3 heteroatoms selected from O, N, and S, optionally substituted with one to three R₇.

In some embodiments of the formulae above, R₃ is (C₁-C₆)alkyl optionally substituted with one to three R₄, and each R₄ is (C₃-C₈)cycloalkyl optionally substituted with one to three R₇.

In some embodiments of the formulae above, R₃ is (C₁-C₆)alkyl substituted with one to three R₄, and each R₄ is (C₃-C₈)cycloalkyl optionally substituted with one to three R₇.

In some embodiments of the formulae above, R₃ is (C₁-C₆)alkyl optionally substituted with one to three R₄, and each R₄ is 5- to 7-membered heterocycloalkyl comprising 1 to 3 heteroatoms selected from O, N, and S, optionally substituted with one to three R₇.

In some embodiments of the formulae above, R₃ is (C₁-C₆)alkyl substituted with one to three R₄, and each R₄ is 5- to 7-membered heterocycloalkyl comprising 1 to 3 heteroatoms selected from O, N, and S, optionally substituted with one to three R₇.

Embodiment 1: A compound of Formula (I), wherein:
R₁ is (C₁-C₆)alkyl, (C₁-C₆)alkoxy, (C₁-C₆)haloalkyl, (C₁-C₆)haloalkoxy, (C₁-C₆)hydroxyalkyl, halogen,
   —OH, —(CH₂)₀₋₂NH₂, —(CH₂)₀₋₂NH(C₁-C₆)alkyl, —(CH₂)₀₋₂N((C₁-C₆)alkyl)₂, —C(O)NH₂, —C(O)OH or CN;
each R₂ is independently (C₁-C₆)alkyl, (C₁-C₆)haloalkyl, (C₁-C₆)hydroxyalkyl, CN, or halogen, or R₁ and R₂ together with the carbon atoms to which they are attached form a (C₃-C₇)cycloalkyl or
   a 4- to 6-membered heterocycloalkyl ring comprising 1 to 3 heteroatoms selected from O, N, and S, or
two R₂ together with the carbon atoms to which they are attached form (C₃-C₇)cycloalkyl or a 4- to 6-membered heterocycloalkyl ring comprising 1 to 3 heteroatoms selected from O, N, and S;
R₃ is (C₁-C₆)alkyl, (C₆-C₁₀)aryl, 5- or 6-membered heteroaryl comprising 1 to 3 heteroatoms selected from O, N, and S, (C₃-C₈)cycloalkyl, or 4- to 7-membered heterocycloalkyl comprising 1 to 3 heteroatoms selected from O, N, and S, wherein the alkyl is optionally substituted with one or more R₄; and the aryl, heteroaryl, cycloalkyl, and heterocycloalkyl are optionally substituted with one or more R₅, or
R₂ and R₃, when on adjacent atoms, together with the atoms to which they are attached form a 5- or 6-membered heterocycloalkyl ring;
each R₄ is independently selected from —C(O)OR₆, —C(O)NR₆R₆′, —NR₆C(O)R₆′, halogen, —OH, —NH₂, CN,
   (C₆-C₁₀)aryl, 5- or 6-membered heteroaryl comprising 1 to 4 heteroatoms selected from O, N, and S,
   (C₃-C₈)cycloalkyl, and 4- to 7-membered heterocycloalkyl ring comprising 1 to 3 heteroatoms selected from O, N, and S, wherein the aryl, heteroaryl, cycloalkyl, and heterocycloalkyl groups are optionally substituted with one or more R₇;
each R₅ is independently selected from (C₁-C₆)alkyl, (C₂-C₆)alkenyl, (C₂-C₆)alkynyl, (C₁-C₆)alkoxy,
   (C₁-C₆)haloalkyl, (C₁-C₆)haloalkoxy, (C₁-C₆)hydroxyalkyl, halogen, —OH, —NH₂, CN, (C₃-C₇)cycloalkyl, 5- to 7-membered heterocycloalkyl comprising 1 to 3 heteroatoms selected from O, N, and S, (C₆-C₁₀)aryl, and 5- or 6-membered heteroaryl comprising 1 to 3 heteroatoms selected from O, N, and S, or
two R₅, when on adjacent atoms, together with the atoms to which they are attached form a (C₆-C₁₀)aryl ring or a 5- or 6-membered heteroaryl ring comprising 1 to 3 heteroatoms selected from O, N, and S, optionally substituted with one or more R₁₀, or
two R₅ together with the atoms to which they are attached form a (C₃-C₇)cycloalkyl ring or a 4- to 7-membered heterocycloalkyl ring comprising 1 to 3 heteroatoms selected from O, N, and S optionally substituted with one or more R₁₀;
R₆ and R₆′ are each independently H, (C₁-C₆)alkyl, or (C₆-C₁₀)aryl;
each R₇ is independently selected from (C₁-C₆)alkyl, (C₂-C₆)alkenyl, (C₂-C₆)alkynyl, (C₁-C₆)alkoxy,
   (C₁-C₆)haloalkyl, (C₁-C₆)haloalkoxy, —C(O)R₈, —(CH₂)₀₋₃C(O)OR₈, —C(O)NR₈R₉, —NR₈C(O)R₉, —NR₈C(O)OR₉, —S(O)ₚNR₈R₉, —S(O)ₚR₁₂, (C₁-C₆)hydroxyalkyl, halogen, —OH, —O(CH₂)₁₋₃CN, —NH₂, CN, —O(CH₂)₀₋₃(C₆-C₁₀)aryl, adamantyl, —O(CH₂)₀₋₃-5- or 6-membered heteroaryl comprising 1 to 3 heteroatoms selected from O, N, and S, (C₆-C₁₀)aryl, monocyclic or bicyclic 5- to 10-membered heteroaryl comprising 1 to 3 heteroatoms selected from O, N, and S, (C₃-C₇)cycloalkyl, and 5- to 7-membered heterocycloalkyl comprising 1 to 3 heteroatoms selected from O, N, and S, wherein the alkyl is optionally substituted with one or more R₁, and the aryl, heteroaryl, and heterocycloalkyl are optionally substituted with one or more substituents each independently selected from halogen,
   (C₁-C₆)alkyl, (C₁-C₆)haloalkyl, and (C₁-C₆)alkoxy, or
two R₇ together with the carbon atom to which they are attached form a ═(O), or
two R₇, when on adjacent atoms, together with the atoms to which they are attached form a (C₆-C₁₀)aryl ring or a 5- or 6-membered heteroaryl ring comprising 1 to 3 heteroatoms selected from O, N, and S, optionally substituted with one or more R₁₀, or
two R₇ together with the atoms to which they are attached form a (C₅-C₇) cycloalkyl ring or a 5- to 7-membered heterocycloalkyl ring comprising 1 to 3 heteroatoms selected from O, N, and S, optionally substituted with one or more R₁₀;
R₈ and R₉ are each independently H or (C₁-C₆)alkyl;
each R₁₀ is independently selected from (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —NH₂, and CN, or
two R₁₀ together with the carbon atom to which they are attached form a ═(O);
each R₁₁ is independently selected from CN, (C₁-C₆)alkoxy, (C₆-C₁₀)aryl, and 5- to 7-membered heterocycloalkyl comprising 1 to 3 heteroatoms selected from O, N, and S, wherein the aryl and heterocycloalkyl are optionally substituted with one or more substituents each independently selected from (C₁-C₆)alkyl, (C₁-C₆)alkoxy, (C₁-C₆)haloalkyl, (C₁-C₆)haloalkoxy, (C₁-C₆)hydroxyalkyl, halogen, —OH, —NH₂, and CN;
R₁₂ is (C₁-C₆)alkyl, (C₁-C₆)haloalkyl, (C₆-C₁₀)aryl, or 5- to 7-membered heterocycloalkyl comprising 1 to 3 heteroatoms selected from O, N, and S;
Rₓ is H or D; and
n is 0, 1, 2, or 3;
   or a pharmaceutically acceptable salt, hydrate, solvate, prodrug, stereoisomer, or tautomer thereof.

Embodiment 2: The compound according to Embodiment 1, wherein Rₓ is H.

Embodiment 3: The compound according to Embodiment 1 or 2, wherein R₁ is (C₁-C₆)alkoxy, halogen, —OH, —(CH₂)₀₋₂NH₂, or CN.

Embodiment 4: The compound according to any one of Embodiments 1-3, wherein R₃ is (C₁-C₆)alkyl optionally substituted with one to three R₄.

Embodiment 5: The compound according to any one of Embodiments 1-3, wherein R₃ is (C₁-C₆)alkyl substituted with one to three R₄.

Embodiment 6: The compound according to any one of Embodiments 1-5, wherein R₄ is selected from (C₆-C₁₀)aryl and 5- or 6-membered heteroaryl comprising 1 to 4 heteroatoms selected from O, N, and S, wherein the aryl and heteroaryl are optionally substituted with one to three R₆.

Embodiment 7: The compound according to any one of Embodiments 1-6, wherein R₄ is phenyl optionally substituted with one to three R₆.

Embodiment 8: The compound according to any one of Embodiments 1-6, wherein R₄ is 5- or 6-membered heteroaryl comprising 1 to 4 heteroatoms selected from O, N, and S, wherein the aryl and heteroaryl are optionally substituted with one to three R₆.

Embodiment 9: The compound according to any one of Embodiments 1-8, wherein n is 0.

Embodiment 10: The compound according to Embodiment 1, having a Formula (Ia) or Formula (Ib):

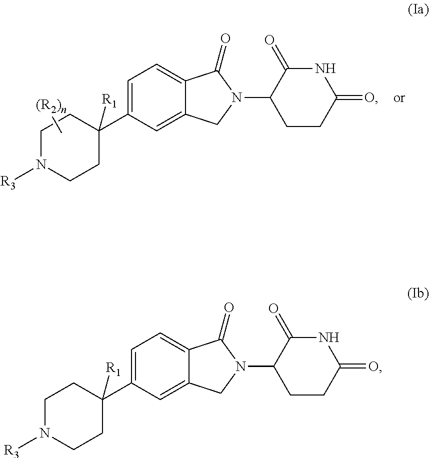

or a pharmaceutically acceptable salt, hydrate, solvate, prodrug, stereoisomer, or tautomer thereof.

Embodiment 11: The compound according to Embodiment 10, wherein R₃ is (C₁-C₆)alkyl optionally substituted with one to three R₄.

Embodiment 12: The compound according to Embodiment 10, wherein R₃ is (C₁-C₆)alkyl substituted with one to three R₄.

Embodiment 13: The compound according to any one of Embodiments 10-12, wherein R₄ is selected from (C₆-C₁₀)aryl and 5- or 6-membered heteroaryl comprising 1 to 4 heteroatoms selected from O, N, and S, wherein the aryl and heteroaryl are optionally substituted with one to three R₆.

Embodiment 14: The compound according to any one of Embodiments 10-13, wherein R₄ is phenyl optionally substituted with one to three R₆.

Embodiment 15: The compound according to any one of Embodiments 10-13, wherein R₄ is 5- or 6-membered heteroaryl comprising 1 to 4 heteroatoms selected from O, N, and S, wherein the aryl and heteroaryl are optionally substituted with one to three R₆.

Embodiment 16: The compound according to Embodiment 1 selected from:
3-(5-(1-benzyl-4-hydroxypiperidin-4-yl)-1-oxoisoindolin-2-yl)piperidine-2,6-dione;
3-(5-(1-benzyl-4-methoxypiperidin-4-yl)-1-oxoisoindolin-2-yl)piperidine-2,6-dione;
3-(5-(1-benzyl-4-fluoropiperidin-4-yl)-1-oxoisoindolin-2-yl)piperidine-2,6-dione;
1-benzyl-4-(2-(2,6-dioxopiperidin-3-yl)-1-oxoisoindolin-5-yl)piperidine-4-carbonitrile;
3-(5-(4-amino-1-benzylpiperidin-4-yl)-1-oxoisoindolin-2-yl)piperidine-2,6-dione;
3-(5-(3-benzyl-3-azabicyclo[4.1.0]heptan-6-yl)-1-oxoisoindolin-2-yl)piperidine-2,6-dione;
3-(5-(3-(((1r,4r)-4-methoxycyclohexyl)methyl)-3-azabicyclo[4.1.0]heptan-6-yl)-1-oxoisoindolin-2-yl)piperidine-2,6-dione;
3-(5-(4-fluoro-1-(((1r,4r)-4-methoxycyclohexyl)methyl)piperidin-4-yl)-1-oxoisoindolin-2-yl)piperidine-2,6-dione;
3-(5-(4-hydroxy-1-(((1r,4r)-4-methoxycyclohexyl)methyl)piperidin-4-yl)-1-oxoisoindolin-2-yl)piperidine-2,6-dione;
3-(5-(4-methoxy-1-(((1r,4r)-4-methoxycyclohexyl)methyl)piperidin-4-yl)-1-oxoisoindolin-2-yl)piperidine-2,6-dione;
3-(5-(4-amino-1-(((1r,4r)-4-methoxycyclohexyl)methyl)piperidin-4-yl)-1-oxoisoindolin-2-yl)piperidine-2,6-dione; and
4-(2-(2,6-dioxopiperidin-3-yl)-1-oxoisoindolin-5-yl)-1-(((1r,4r)-4-methoxycyclohexyl)methyl)piperidine-4-carbonitrile;
   or a pharmaceutically acceptable salt, hydrate, solvate, prodrug, stereoisomer, or tautomer thereof.

Embodiment 17: A compound selected from:
3-(1-oxo-5-(1-((6-oxo-1,6-dihydropyridin-3-yl)methyl)piperidin-4-yl)isoindolin-2-yl)piperidine-2,6-dione;
3-(5-(1-benzyl-2,6-dimethylpiperidin-4-yl)-1-oxoisoindolin-2-yl)piperidine-2,6-dione;
(1r,4r)-4-((4-(2-(2,6-dioxopiperidin-3-yl)-1-oxoisoindolin-5-yl)piperidin-1-yl)methyl)cyclohexane-1-carbonitrile;
3-(5-(2,6-dimethylpiperidin-4-yl)-1-oxoisoindolin-2-yl)piperidine-2,6-dione;
3-(5-(1-((5-ethoxypyridin-2-yl)methyl)piperidin-4-yl)-1-oxoisoindolin-2-yl)piperidine-2,6-dione;
3-(5-(1-((5-methoxypyridin-2-yl)methyl)piperidin-4-yl)-1-oxoisoindolin-2-yl)piperidine-2,6-dione;
3-(5-(1-((6-methoxypyridin-3-yl)methyl)piperidin-4-yl)-1-oxoisoindolin-2-yl)piperidine-2,6-dione;
3-(5-(1-((6-ethoxypyridin-3-yl)methyl)piperidin-4-yl)-1-oxoisoindolin-2-yl)piperidine-2,6-dione;
3-(5-(1-((5-methyl-TH-imidazol-4-yl)methyl)piperidin-4-yl)-1-oxoisoindolin-2-yl)piperidine-2,6-dione;
3-(5-(1-((4-(fluoromethyl)cyclohexyl)methyl)piperidin-4-yl)-1-oxoisoindolin-2-yl)piperidine-2,6-dione;
3-(5-(1-(2-(1H-pyrazol-1-yl)ethyl)piperidin-4-yl)-1-oxoisoindolin-2-yl)piperidine-2,6-dione
3-(5-(1-((4-methylpyrimidin-5-yl)methyl)piperidin-4-yl)-1-oxoisoindolin-2-yl)piperidine-2,6-dione;
3-(5-(1-((4-methylcyclohex-3-en-1-yl)methyl)piperidin-4-yl)-1-oxoisoindolin-2-yl)piperidine-2,6-dione;
3-(1-oxo-5-(1-(pyrazin-2-ylmethyl)piperidin-4-yl)isoindolin-2-yl)piperidine-2,6-dione;
3-(1-oxo-5-(1-(pyridazin-3-ylmethyl)piperidin-4-yl)isoindolin-2-yl)piperidine-2,6-dione;
3-(1-oxo-5-(1-(pyrimidin-4-ylmethyl)piperidin-4-yl)isoindolin-2-yl)piperidine-2,6-dione;
3-(5-(1-((2-methylpyrimidin-5-yl)methyl)piperidin-4-yl)-1-oxoisoindolin-2-yl)piperidine-2,6-dione;
3-(1-oxo-5-(1-(pyridazin-4-ylmethyl)piperidin-4-yl)isoindolin-2-yl)piperidine-2,6-dione;
3-(5-((1R,4S)-2-benzyl-2-azabicyclo[2.2.2]octan-5-yl)-1-oxoisoindolin-2-yl)piperidine-2,6-dione;
3-(5-((1R,5S)-9-benzyl-3-methyl-3,9-diazabicyclo[3.3.1]nonan-7-yl)-1-oxoisoindolin-2-yl)piperidine-2,6-dione;
3-(5-((1R,5S)-9-benzyl-3-oxa-9-azabicyclo[3.3.1]nonan-7-yl)-1-oxoisoindolin-2-yl)piperidine-2,6-dione;
3-(5-((1R,5S)-9-benzyl-3,9-diazabicyclo[3.3.1]nonan-7-yl)-1-oxoisoindolin-2-yl)piperidine-2,6-dione;
3-(5-((1S,4S)-2-benzyl-2-azabicyclo[2.2.1]heptan-5-yl)-1-oxoisoindolin-2-yl)piperidine-2,6-dione;
3-(5-((1R,5S)-9-ethyl-3-methyl-3,9-diazabicyclo[3.3.1]nonan-7-yl)-1-oxoisoindolin-2-yl)piperidine-2,6-dione;
3-(1-oxo-5-(1-(1-(tetrahydro-2H-pyran-4-yl)ethyl)piperidin-4-yl)isoindolin-2-yl)piperidine-2,6-dione;
3-(5-(1-(1-(1-ethyl-1H-pyrazol-4-yl)ethyl)piperidin-4-yl)-1-oxoisoindolin-2-yl)piperidine-2,6-dione;
3-(5-(1-(1-(1-ethyl-1H-pyrazol-4-yl)propyl)piperidin-4-yl)-1-oxoisoindolin-2-yl)piperidine-2,6-dione;
3-(1-oxo-5-(1-(1-(pyrazin-2-yl)propyl)piperidin-4-yl)isoindolin-2-yl)piperidine-2,6-dione;
3-(1-oxo-5-(1-(1-(pyridazin-4-yl)propyl)piperidin-4-yl)isoindolin-2-yl)piperidine-2,6-dione;
3-(5-(1-(6,7-dihydro-5H-cyclopenta[b]pyridin-5-yl)piperidin-4-yl)-1-oxoisoindolin-2-yl)piperidine-2,6-dione;
3-(5-(1-(4-methoxycyclohexyl)piperidin-4-yl)-1-oxoisoindolin-2-yl)piperidine-2,6-dione;
3-(1-oxo-5-(1-(2-(pyridin-4-yl)propan-2-yl)piperidin-4-yl)isoindolin-2-yl)piperidine-2,6-dione; and
3-(1-oxo-5-(1-((2-oxo-1,2-dihydropyridin-3-yl)methyl)piperidin-4-yl)isoindolin-2-yl)piperidine-2,6-dione;
   or a pharmaceutically acceptable salt, hydrate, solvate, prodrug, stereoisomer, or tautomer thereof.

Embodiment 18: A pharmaceutical composition comprising a therapeutically effective amount of a compound according to any one of Embodiments 1-17, or a pharmaceutically acceptable salt, hydrate, solvate, prodrug, stereoisomer, or tautomer thereof, and a pharmaceutically acceptable carrier or excipient.

Embodiment 19: The pharmaceutical composition according to Embodiment 18 further comprising at least one additional pharmaceutical agent.

Embodiment 20: The pharmaceutical composition according to Embodiment 18 or Embodiment 19 for use in the treatment of a disease or disorder that is affected by the reduction of IKZF2 protein levels.

Embodiment 21: A method of degrading IKZF2 comprising administering to the patient in need thereof a compound according to any one of Embodiments 1-17, or a pharmaceutically acceptable salt, hydrate, solvate, prodrug, stereoisomer, or tautomer thereof.

Embodiment 22: A method of treating a disease or disorder that is affected by the modulation of IKZF2 protein levels comprising administering to the patient in need thereof a compound according to any one of Embodiments 1-17, or a pharmaceutically acceptable salt, hydrate, solvate, prodrug, stereoisomer, or tautomer thereof.

Embodiment 23: A method of modulating IKZF2 protein levels comprising administering to the patient in need thereof a compound according to any one of Embodiments 1-17, or a pharmaceutically acceptable salt, hydrate, solvate, prodrug, stereoisomer, or tautomer thereof.

Embodiment 24: A method of reducing the proliferation of a cell the method comprising, contacting the cell with a compound according to any one of Embodiments 1-17, or a pharmaceutically acceptable salt, hydrate, solvate, prodrug, stereoisomer, or tautomer thereof, and reducing IKZF2 protein levels.

Embodiment 25: A method of treating cancer comprising administering to the patient in need thereof a compound according to any one of Embodiments 1-17, or a pharmaceutically acceptable salt, hydrate, solvate, prodrug, stereoisomer, or tautomer thereof.

Embodiment 26: The method according to Embodiment 25, wherein the cancer is selected from non-small cell lung cancer (NSCLC), melanoma, triple-negative breast cancer (TNBC), nasopharyngeal cancer (NPC), microsatellite stable colorectal cancer (mssCRC), thymoma, carcinoid, acute myelogenous leukemia, and gastrointestinal stromal tumor (GIST).

Embodiment 27: The method according to Embodiment 25, wherein the cancer is a cancer for which the immune response is deficient or an immunogenic cancer.

Embodiment 28: A method for reducing IKZF2 protein levels in a subject comprising the step of administering to a subject in need thereof a therapeutically effective amount of a compound according to any one of Embodiments 1-17, or a pharmaceutically acceptable salt.

Embodiment 29: The method according to any one of Embodiments 21-28, wherein administering is performed orally, parentally, subcutaneously, by injection, or by infusion.

Embodiment 30: A compound according to any one of Embodiments 1-17, or a pharmaceutically acceptable salt, hydrate, solvate, prodrug, stereoisomer, or tautomer thereof, for use in the treatment of a disease or disorder that is affected by the reduction of IKZF2 protein levels.

Embodiment 31: Use of a compound according to any one of claims 1-17, or a pharmaceutically acceptable salt, hydrate, solvate, prodrug, stereoisomer, or tautomer thereof, in the manufacture of a medicament for treating a disease or disorder that is affected by the reduction of IKZF2 protein levels.

Embodiment 32: A compound according to any one of Embodiments 1-17, or a pharmaceutically acceptable salt, hydrate, solvate, prodrug, stereoisomer, or tautomer thereof, for use in the manufacture of a medicament for treating a disease or disorder associated with the reduction of IKZF2 protein levels.

Embodiment 33: Use of a compound according to any one of Embodiments 1-17, or a pharmaceutically acceptable salt, hydrate, solvate, prodrug, stereoisomer, or tautomer thereof, in the treatment of a disease or disorder associated with the reduction of IKZF2 protein levels.

Embodiment 34: The compound according to Embodiment 30 or 32 or the use according to Embodiment 31 or 33, wherein the disease or disorder is selected from non-small cell lung cancer (NSCLC), melanoma, triple-negative breast cancer (TNBC), nasopharyngeal cancer (NPC), microsatellite stable colorectal cancer (mssCRC), thymoma, carcinoid, acute myelogenous leukemia, and gastrointestinal stromal tumor (GIST).

Embodiment 35: A compound selected from:

| Cmpd | | |
|---|---|---|
| No. | Compound Structure | Compound Name |
| I-1 | 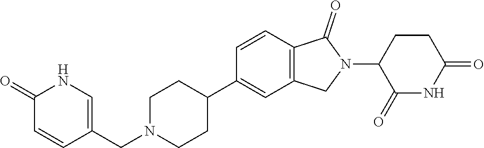 [SMILES: O=C1CCC(N2Cc3cc(C4CCN(Cc5ccc(=O)[nH]c5)CC4)ccc3C2=O)C(=O)N1] | 3-(1-oxo-5-(1-((6-oxo-1,6- dihydropyridin-3- yl)methyl)piperidin-4- yl)isoindolin-2-yl)piperidine- 2,6-dione; |
| I-2 | 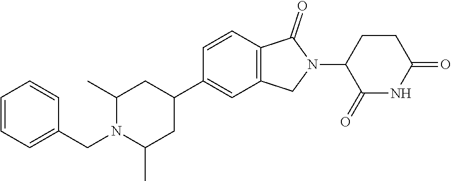 [SMILES: CC1CC(c2ccc3c(c2)CN(C2CCC(=O)NC2=O)C3=O)CC(C)N1Cc1ccccc1] | 3-(5-(1-benzyl-2,6- dimethylpiperidin-4-yl)-1- oxoisoindolin-2-yl)piperidine- 2,6-dione; |
| I-3 | 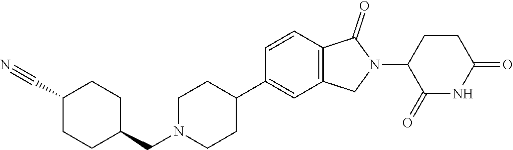 [SMILES: N#C[C@H]1CC[C@H](CN2CCC(c3ccc4c(c3)CN(C3CCC(=O)NC3=O)C4=O)CC2)CC1] | (1r,4r)-4-((4-(2-(2,6- dioxopiperidin-3-yl)-1- oxoisoindolin-5-yl)piperidin-1- yl)methyl)cyclohexane-1- carbonitrile; |
| I-4 | 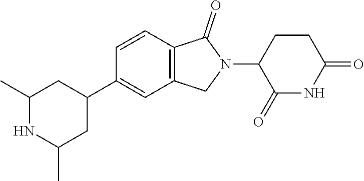 [SMILES: CC1CC(c2ccc3c(c2)CN(C2CCC(=O)NC2=O)C3=O)CC(C)N1] | 3-(5-(2,6-dimethylpiperidin-4- yl)-1-oxoisoindolin-2- yl)piperidine-2,6-dione; |
| I-5 | 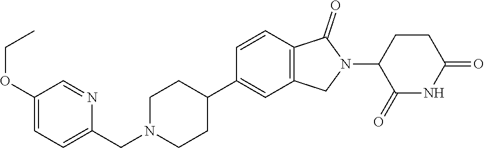 [SMILES: CCOc1ccc(CN2CCC(c3ccc4c(c3)CN(C3CCC(=O)NC3=O)C4=O)CC2)nc1] | 3-(5-(1-((5-ethoxypyridin-2- yl)methyl)piperidin-4-yl)-1- oxoisoindolin-2-yl)piperidine- 2,6-dione; |
| I-6 | 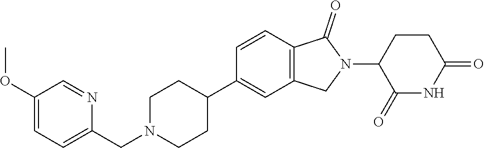 [SMILES: COc1ccc(CN2CCC(c3ccc4c(c3)CN(C3CCC(=O)NC3=O)C4=O)CC2)nc1] | 3-(5-(1-((5-methoxypyridin-2- yl)methyl)piperidin-4-yl)-1- oxoisoindolin-2-yl)piperidine- 2,6-dione; |
| I-7 | 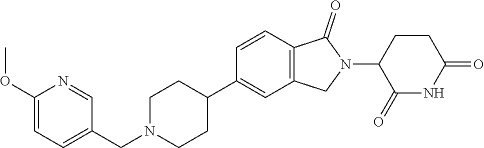 [SMILES: COc1ccc(CN2CCC(c3ccc4c(c3)CN(C3CCC(=O)NC3=O)C4=O)CC2)cn1] | 3-(5-(1-((6-methoxypyridin-3- yl)methyl)piperidin-4-yl)-1- oxoisoindolin-2-yl)piperidine- 2,6-dione; |
| I-8 | 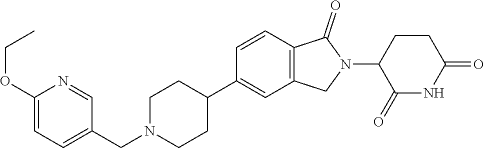 [SMILES: CCOc1ccc(CN2CCC(c3ccc4c(c3)CN(C3CCC(=O)NC3=O)C4=O)CC2)cn1] | 3-(5-(1-((6-ethoxypyridin-3- yl)methyl)piperidin-4-yl)-1- oxoisoindolin-2-yl)piperidine- 2,6-dione; |
| I-9 | 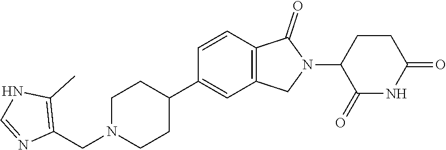 [SMILES: Cc1[nH]cnc1CN1CCC(c2ccc3c(c2)CN(C2CCC(=O)NC2=O)C3=O)CC1] | 3-(5-(1-((5-methyl-1H-imidazol- 4-yl)methyl)piperidin-4-yl)-1- oxoisoindolin-2-yl)piperidine- 2,6-dione; |
| I-10 | 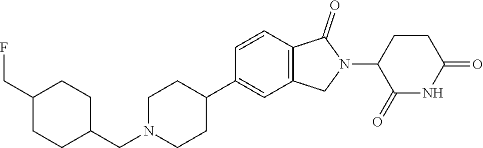 [SMILES: O=C1CCC(N2Cc3cc(C4CCN(CC5CCC(CF)CC5)CC4)ccc3C2=O)C(=O)N1] | 3-(5-(1-((4- (fluoromethyl)cyclohexyl) methyl)piperidin-4-yl)-1- oxoisoindolin-2-yl)piperidine- 2,6-dione; |
| I-11 | 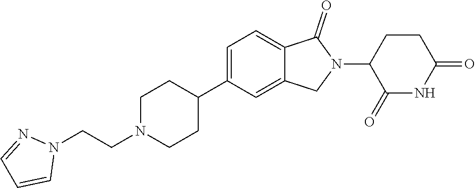 [SMILES: O=C1CCC(N2Cc3cc(C4CCN(CCn5cccn5)CC4)ccc3C2=O)C(=O)N1] | 3-(5-(1-(2-(1H-pyrazol-1- yl)ethyl)piperidin-4-yl)-1- oxoisoindolin-2-yl)piperidine- 2,6-dione; |
| I-12 | 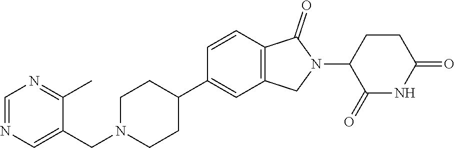 [SMILES: Cc1ncncc1CN1CCC(c2ccc3c(c2)CN(C2CCC(=O)NC2=O)C3=O)CC1] | 3-(5-(1-((4-methylpyrimidin-5- yl)methyl)piperidin-4-yl)-1- oxoisoindolin-2-yl)piperidine- 2,6-dione; |
| I-13 | 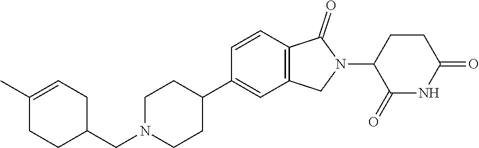 [SMILES: CC1=CCC(CN2CCC(c3ccc4c(c3)CN(C3CCC(=O)NC3=O)C4=O)CC2)CC1] | 3-(5-(1-((4-methylcyclohex-3- en-1-yl)methyl)piperidin-4-yl)- 1-oxoisoindolin-2-yl)piperidine- 2,6-dione; |
| I-14 | 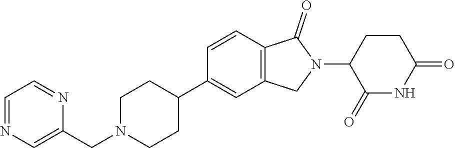 [SMILES: O=C1CCC(N2Cc3cc(C4CCN(Cc5cnccn5)CC4)ccc3C2=O)C(=O)N1] | 3-(1-oxo-5-(1-(pyrazin-2- ylmethyl)piperidin-4- yl)isoindolin-2-yl)piperidine- 2,6-dione; |
| I-15 | 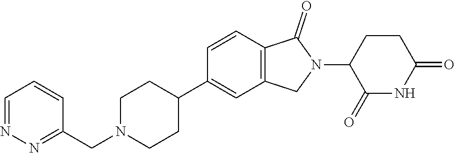 [SMILES: O=C1CCC(N2Cc3cc(C4CCN(Cc5cccnn5)CC4)ccc3C2=O)C(=O)N1] | 3-(1-oxo-5-(1-(pyridazin-3- ylmethyl)piperidin-4- yl)isoindolin-2-yl)piperidine- 2,6-dione; |
| I-16 | 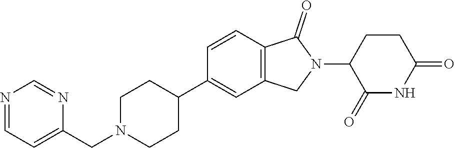 [SMILES: O=C1CCC(N2Cc3cc(C4CCN(Cc5ccncn5)CC4)ccc3C2=O)C(=O)N1] | 3-(1-oxo-5-(1-(pyrimidin-4- ylmethyl)piperidin-4- yl)isoindolin-2-yl)piperidine- 2,6-dione; |
| I-17 | 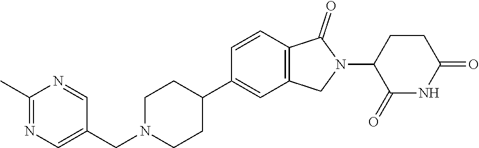 [SMILES: Cc1ncc(CN2CCC(c3ccc4c(c3)CN(C3CCC(=O)NC3=O)C4=O)CC2)cn1] | 3-(5-(1-((2-methylpyrimidin-5- yl)methyl)piperidin-4-yl)-1- oxoisoindolin-2-yl)piperidine- 2,6-dione; |
| I-18 | 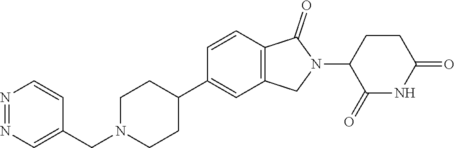 [SMILES: O=C1CCC(N2Cc3cc(C4CCN(Cc5ccnnc5)CC4)ccc3C2=O)C(=O)N1] | 3-(1-oxo-5-(1-(pyridazin-4- ylmethyl)piperidin-4- yl)isoindolin-2-yl)piperidine- 2,6-dione; |
| I-19 | 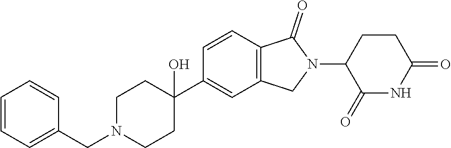 [SMILES: O=C1CCC(N2Cc3cc(C4(O)CCN(Cc5ccccc5)CC4)ccc3C2=O)C(=O)N1] | 3-(5-(1-benzyl-4- hydroxypiperidin-4-yl)-1- oxoisoindolin-2-yl)piperidine- 2,6-dione; |
| I-20 | 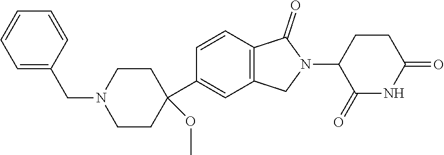 [SMILES: COC1(c2ccc3c(c2)CN(C2CCC(=O)NC2=O)C3=O)CCN(Cc2ccccc2)CC1] | 3-(5-(1-benzyl-4- methoxypiperidin-4-yl)-1- oxoisoindolin-2-yl)piperidine- 2,6-dione; |
| I-21 | 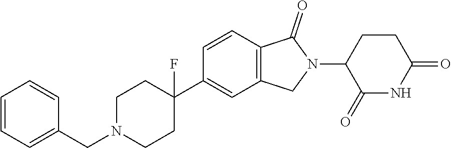 [SMILES: O=C1CCC(N2Cc3cc(C4(F)CCN(Cc5ccccc5)CC4)ccc3C2=O)C(=O)N1] | 3-(5-(1-benzyl-4- fluoropiperidin-4-yl)-1- oxoisoindolin-2-yl)piperidine- 2,6-dione; |
| I-22 | 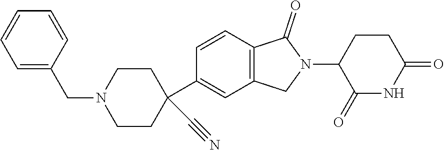 [SMILES: N#CC1(c2ccc3c(c2)CN(C2CCC(=O)NC2=O)C3=O)CCN(Cc2ccccc2)CC1] | 1-benzyl-4-(2-(2,6- dioxopiperidin-3-yl)-1- oxoisoindolin-5-yl)piperidine-4- carbonitrile; |
| I-23 | 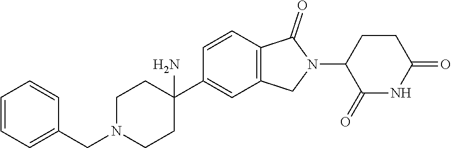 [SMILES: NC1(c2ccc3c(c2)CN(C2CCC(=O)NC2=O)C3=O)CCN(Cc2ccccc2)CC1] | 3-(5-(4-amino-1- benzylpiperidin-4-yl)-1- oxoisoindolin-2-yl)piperidine- 2,6-dione; |
| I-24 | 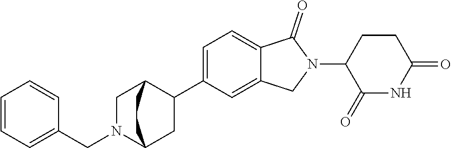 [SMILES: O=C1CCC(N2Cc3cc(C4C[C@H]5CC[C@@H]4CN5Cc4ccccc4)ccc3C2=O)C(=O)N1] | 3-(5-((1R,4S)-2-benzyl-2- azabicyclo[2.2.2]octan-5-yl)-1- oxoisoindolin-2-yl)piperidine- 2,6-dione; |
| I-25 | 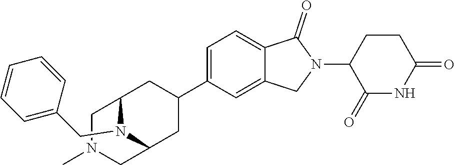 [SMILES: CN1C[C@H]2CC(c3ccc4c(c3)CN(C3CCC(=O)NC3=O)C4=O)C[C@@H](C1)N2Cc1ccccc1] | 3-(5-((1R,5S)-9-benzyl-3- methyl-3,9- diazabicyclo[3.3.1]nonan-7-yl)- 1-oxoisoindolin-2-yl)piperidine- 2,6-dione; |
| I-26 | 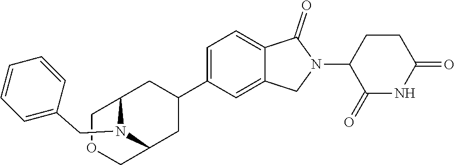 [SMILES: O=C1CCC(N2Cc3cc(C4C[C@H]5COC[C@@H](C4)N5Cc4ccccc4)ccc3C2=O)C(=O)N1] | 3-(5-((1R,5S)-9-benzyl-3-oxa-9- azabicyclo[3.3.1]nonan-7-yl)-1- oxoisoindolin-2-yl)piperidine- 2,6-dione |
| I-27 | 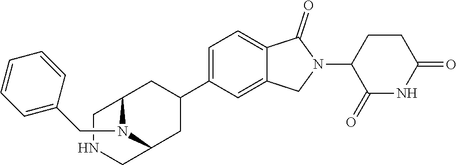 [SMILES: O=C1CCC(N2Cc3cc(C4C[C@H]5CNC[C@@H](C4)N5Cc4ccccc4)ccc3C2=O)C(=O)N1] | 3-(5-((1R,5S)-9-benzyl-3,9- diazabicyclo[3.3.1]nonan-7-yl)- 1-oxoisoindolin-2-yl)piperidine- 2,6-dione; |
| I-28 | 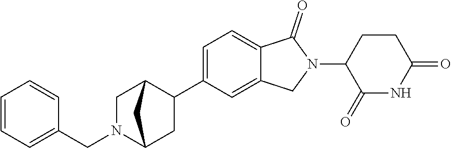 [SMILES: O=C1CCC(N2Cc3cc(C4C[C@H]5C[C@@H]4CN5Cc4ccccc4)ccc3C2=O)C(=O)N1] | 3-(5-((1S,4S)-2-benzyl-2- azabicyclo[2.2.1]heptan-5-yl)-1- oxoisoindolin-2-yl)piperidine- 2,6-dione; |
| I-29 | 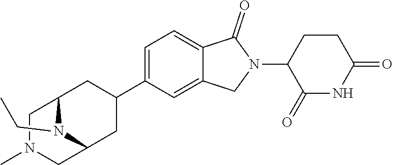 [SMILES: CCN1[C@@H]2CC(c3ccc4c(c3)CN(C3CCC(=O)NC3=O)C4=O)C[C@H]1CN(C)C2] | 3-(5-((1R,5S)-9-ethyl-3-methyl- 3,9-diazabicyclo[3.3.1]nonan-7- yl)-1-oxoisoindolin-2- yl)piperidine-2,6-dione; |
| I-30 | 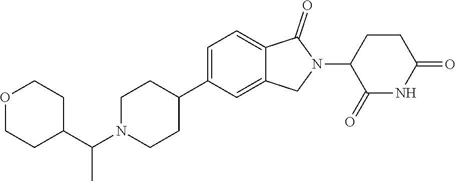 [SMILES: CC(C1CCOCC1)N1CCC(c2ccc3c(c2)CN(C2CCC(=O)NC2=O)C3=O)CC1] | 3-(1-oxo-5-(1-(1-(tetrahydro-2H- pyran-4-yl)ethyl)piperidin-4- yl)isoindolin-2-yl)piperidine- 2,6-dione; |
| I-31 | 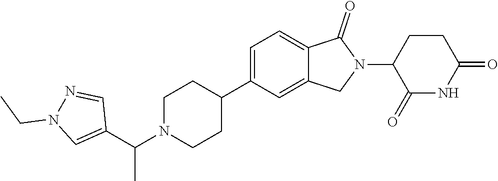 [SMILES: CCn1cc(C(C)N2CCC(c3ccc4c(c3)CN(C3CCC(=O)NC3=O)C4=O)CC2)cn1] | 3-(5-(1-(1-(1-ethyl-1H-pyrazol- 4-yl)ethyl)piperidin-4-yl)-1- oxoisoindolin-2-yl)piperidine- 2,6-dione; |
| I-32 | 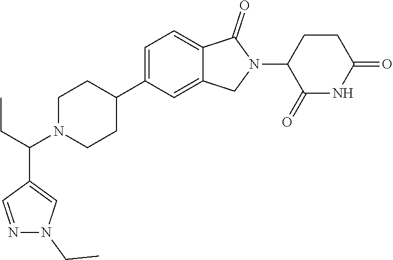 [SMILES: CCC(c1cnn(CC)c1)N1CCC(c2ccc3c(c2)CN(C2CCC(=O)NC2=O)C3=O)CC1] | 3-(5-(1-(1-(1-ethyl-1H-pyrazol- 4-yl)propyl)piperidin-4-yl)-1- oxoisoindolin-2-yl)piperidine- 2,6-dione; |
| I-33 | 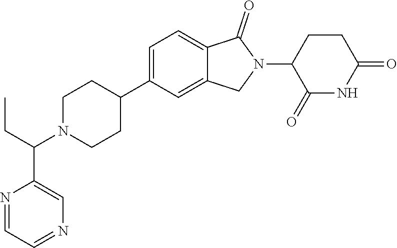 [SMILES: CCC(c1cnccn1)N1CCC(c2ccc3c(c2)CN(C2CCC(=O)NC2=O)C3=O)CC1] | 3-(1-oxo-5-(1-(1-(pyrazin-2- yl)propyl)piperidin-4- yl)isoindolin-2-yl)piperidine- 2,6-dione; |
| I-34 | 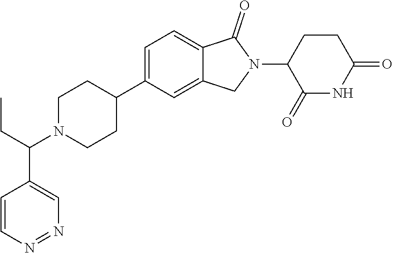 [SMILES: CCC(c1ccnnc1)N1CCC(c2ccc3c(c2)CN(C2CCC(=O)NC2=O)C3=O)CC1] | 3-(1-oxo-5-(1-(1-(pyridazin-4- yl)propyl)piperidin-4- yl)isoindolin-2-yl)piperidine- 2,6-dione; |
| I-35 | 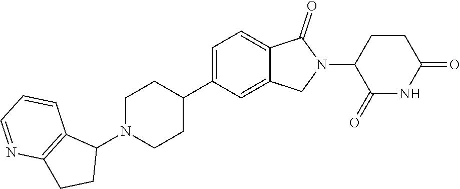 [SMILES: O=C1CCC(N2Cc3cc(C4CCN(C5CCc6ncccc65)CC4)ccc3C2=O)C(=O)N1] | 3-(5-(1-(6,7-dihydro-5H- cyclopenta[b]pyridin-5- yl)piperidin-4-yl)-1- oxoisoindolin-2-yl)piperidine- 2,6-dione; |
| I-36 | 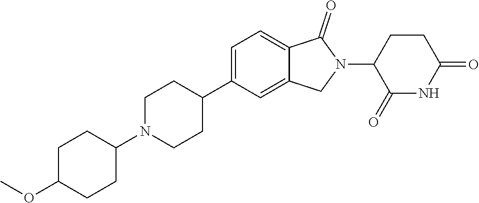 [SMILES: COC1CCC(N2CCC(c3ccc4c(c3)CN(C3CCC(=O)NC3=O)C4=O)CC2)CC1] | 3-(5-(1-(4- methoxycyclohexyl)piperidin-4- yl)-1-oxoisoindolin-2- yl)piperidine-2,6-dione; |
| I-37 | 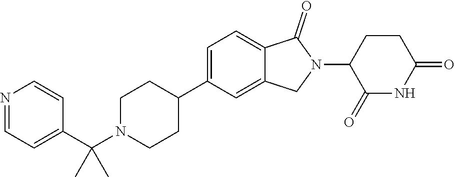 [SMILES: CC(C)(c1ccncc1)N1CCC(c2ccc3c(c2)CN(C2CCC(=O)NC2=O)C3=O)CC1] | 3-(1-oxo-5-(1-(2-(pyridin-4- yl)propan-2-yl)piperidin-4- yl)isoindolin-2-yl)piperidine- 2,6-dione; |
| I-38 | 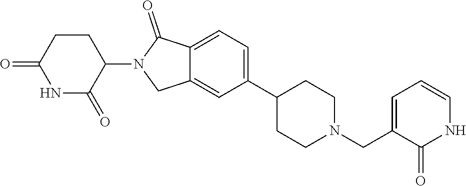 [SMILES: O=C1CCC(N2Cc3cc(C4CCN(Cc5ccc[nH]c5=O)CC4)ccc3C2=O)C(=O)N1] | 3-(1-oxo-5-(1-((2-oxo-1,2- dihydropyridin-3- yl)methyl)piperidin-4- yl)isoindolin-2-yl)piperidine- 2,6-dione; |
| I-39 | 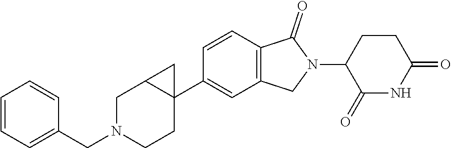 [SMILES: O=C1CCC(N2Cc3cc(C45CCN(Cc6ccccc6)CC4C5)ccc3C2=O)C(=O)N1] | 3-(5-(3-benzyl-3- azabicyclo[4.1.0]heptan-6-yl)-1- oxoisoindolin-2-yl)piperidine- 2,6-dione; |
| I-40 | 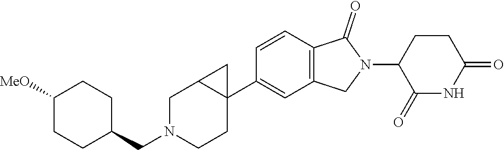 [SMILES: CO[C@H]1CC[C@H](CN2CCC3(c4ccc5c(c4)CN(C4CCC(=O)NC4=O)C5=O)CC3C2)CC1] | 3-(5-(3-(((1r,4r)-4- methoxycyclohexyl)methyl)-3- azabicyclo[4.1.0]heptan-6-yl)-1- oxoisoindolin-2-yl)piperidine- 2,6-dione; |
| I-42 | 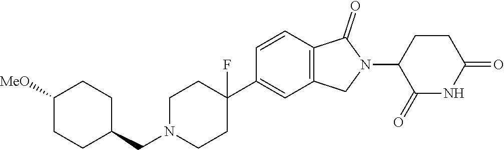 [SMILES: CO[C@H]1CC[C@H](CN2CCC(F)(c3ccc4c(c3)CN(C3CCC(=O)NC3=O)C4=O)CC2)CC1] | 3-(5-(4-fluoro-1-(((1r,4r)-4- methoxycyclohexyl)methyl) piperidin-4-yl)-1- oxoisoindolin-2- yl)piperidine-2,6-dione; |
| I-41 | 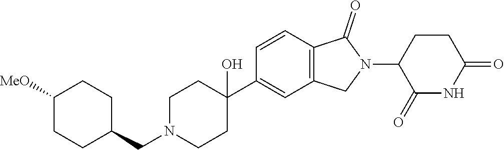 [SMILES: CO[C@H]1CC[C@H](CN2CCC(O)(c3ccc4c(c3)CN(C3CCC(=O)NC3=O)C4=O)CC2)CC1] | 3-(5-(4-hydroxy-1-(((1r,4r)-4- methoxycyclohexyl)methyl) piperidin-4-yl)-1- oxoisoindolin-2- yl)piperidine-2,6-dione; |
| I-43 | 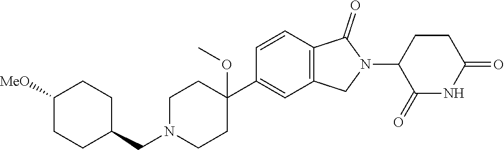 [SMILES: COC1(c2ccc3c(c2)CN(C2CCC(=O)NC2=O)C3=O)CCN(C[C@H]2CC[C@H](OC)CC2)CC1] | 3-(5-(4-methoxy-1-(((1r,4r)-4- methoxycyclohexyl)methyl) piperidin-4-yl)-1- oxoisoindolin-2- yl)piperidine-2,6-dione; |
| I-44 | 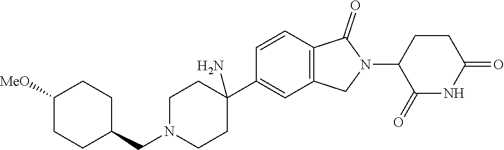 [SMILES: CO[C@H]1CC[C@H](CN2CCC(N)(c3ccc4c(c3)CN(C3CCC(=O)NC3=O)C4=O)CC2)CC1] | 3-(5-(4-amino-1-(((1r,4r)-4- methoxycyclohexyl)methyl) piperidin-4-yl)-1- oxoisoindolin-2- yl)piperidine-2,6-dione; and |
| I-45 | 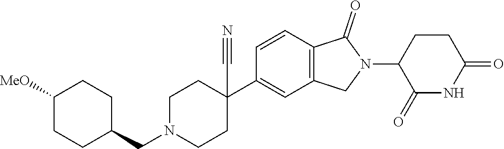 [SMILES: CO[C@H]1CC[C@H](CN2CCC(C#N)(c3ccc4c(c3)CN(C3CCC(=O)NC3=O)C4=O)CC2)CC1] | 4-(2-(2,6-dioxopiperidin-3-yl)-1- oxoisoindolin-5-yl)-1-(((1r,4r)- 4- methoxycyclohexyl)methyl) piperidine-4-carbonitrile. |

In another embodiment of the disclosure, the compounds of the present disclosure are enantiomers. In some embodiments the compounds are the (S)-enantiomer. In other embodiments the compounds are the (R)-enantiomer. In yet other embodiments, the compounds of the present disclosure may be (+) or (−) enantiomers.

It should be understood that all isomeric forms are included within the present disclosure, including mixtures thereof. If the compound contains a double bond, the substituent may be in the E or Z configuration. If the compound contains a disubstituted cycloalkyl, the cycloalkyl substituent may have a cis- or trans configuration. All tautomeric forms are also intended to be included.

Compounds of the disclosure, and pharmaceutically acceptable salts, hydrates, solvates, stereoisomers, and prodrugs thereof may exist in their tautomeric form (for example, as an amide or imino ether). All such tautomeric forms are contemplated herein as part of the present disclosure.

The compounds of the disclosure may contain asymmetric or chiral centers and, therefore, exist in different stereoisomeric forms. It is intended that all stereoisomeric forms of the compounds of the disclosure as well as mixtures thereof, including racemic mixtures, form part of the present disclosure. In addition, the present disclosure embraces all geometric and positional isomers. For example, if a compound of the disclosure incorporates a double bond or a fused ring, both the cis- and trans-forms, as well as mixtures, are embraced within the scope of the disclosure. Each compound herein disclosed includes all the enantiomers that conform to the general structure of the compound. The compounds may be in a racemic or enantiomerically pure form, or any other form in terms of stereochemistry. The assay results may reflect the data collected for the racemic form, the enantiomerically pure form, or any other form in terms of stereochemistry.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of the disclosure may be atropisomers (e.g., substituted biaryls) and are considered as part of this disclosure. Enantiomers can also be separated by use of a chiral HPLC column.

It is also possible that the compounds of the disclosure may exist in different tautomeric forms, and all such forms are embraced within the scope of the disclosure and chemical structures and names. Also, for example, all keto-enol and imine-enamine forms of the compounds are included in the disclosure.

All stereoisomers (for example, geometric isomers, optical isomers, and the like) of the present compounds (including those of the salts, solvates, esters, and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this disclosure, as are positional isomers (such as, for example, 4-pyridyl and 3-pyridyl). (For example, if a compound of Formula (I) or a compound of Embodiment 17 incorporates a double bond or a fused ring, both the cis- and trans-forms, as well as mixtures, are embraced within the scope of the disclosure. Also, for example, all keto-enol and imine-enamine forms of the compounds are included in the disclosure.) Individual stereoisomers of the compounds of the disclosure may, for example, be substantially free of other isomers, or is admixed, for example, as racemates or with all other, or other selected, stereoisomers.

The chiral centers of the compounds of the disclosure can have the S or R configuration as defined by the IUPAC 1974 Recommendations. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated double bonds may, if possible, be present in cis-(Z)- or trans-(E)-form.

The use of the terms “salt”, “solvate”, “ester,” “prodrug”, and the like, is intended to equally apply to the salt, solvate, ester, and prodrug of enantiomers, stereoisomers, rotamers, tautomers, positional isomers, racemates, or prodrugs of the inventive compounds.

The compounds of the disclosure may form salts which are also within the scope of this disclosure. Reference to a compound of the Formula herein is generally understood to include reference to salts thereof, unless otherwise indicated.

The compounds and intermediates may be isolated and used as the compound per se. Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine, and, such as ²H, ³H, ¹¹C, ¹³C ¹⁴C, ¹⁵N, ¹⁸F, ³¹P ³²P, respectively. The disclosure includes various isotopically labeled compounds as defined herein, for example those into which radioactive isotopes, such as ³H, ¹³C, and ¹⁴C, are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F, ¹¹C or labeled compound may be particularly desirable for PET or SPECT studies.

Further, substitution with heavier isotopes, particularly deuterium (i.e., ²H or D) may afford certain therapeutic advantages resulting from greater metabolic stability, for example, increased in vivo half-life, reduced dosage requirements, reduced CYP450 inhibition (competitive or time dependent) or an improvement in therapeutic index. For example, substitution with deuterium may modulate undesirable side effects of the undeuterated compound, such as competitive CYP450 inhibition, time dependent CYP450 inactivation, etc. It is understood that deuterium in this context is regarded as a substituent in compounds of the present disclosure. The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound of this disclosure is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Isotopically-labeled compounds of the present disclosure can generally be prepared by conventional techniques known to those skilled in the art or by carrying out the procedures disclosed in the schemes or in the examples and preparations described below using an appropriate isotopically-labeled reagent in place of the non-isotopically labeled reagent.

Pharmaceutically acceptable solvates in accordance with the disclosure include those wherein the solvent of crystallization may be isotopically substituted, e.g., D₂O, d₆-acetone, d₆-DMSO.

The present disclosure relates to compounds which are modulators of IKZF2 protein levels. In one embodiment, the compounds of the present disclosure decrease IKZF2 protein levels. In yet one embodiment, the compounds of the present disclosure reduce IKZF2 protein levels. In another embodiment, the compounds of the present disclosure are degraders of IKZF2.

The present disclosure relates to compounds, which are modulators of IKZF2 and IKZF4 protein levels. In one embodiment, the compounds of the present disclosure decrease IKZF2 and IKZF4 protein levels. In yet one embodiment, the compounds of the present disclosure reduce IKZF2 and IKZF4 protein levels. In another embodiment, the compounds of the present disclosure are degraders of IKZF2.

In some embodiments, the compounds of the disclosure are selective over other proteins. As used herein “selective modulator”, “selective degrader”, or “selective compound” means, for example, a compound of the disclosure, that effectively modulates, decreases, or reduces the levels of a specific protein or degrades a specific protein to a greater extent than any other protein. A “selective modulator”, “selective degrader”, or “selective compound” can be identified, for example, by comparing the ability of a compound to modulate, decrease, or reduce the levels of or to degrade a specific protein to its ability to modulate, decrease, or reduce the levels of or to degrade other proteins. In some embodiments, the selectivity can be identified by measuring the AC₅₀, EC₅₀, or IC₅₀ of the compounds.

In some embodiments, the compounds of the present application are selective IKZF2 modulators. As used herein “selective IKZF2 modulator”, “selective IKZF2 degrader”, or “selective IKZF2 compound” refers to a compound of the application, for example, that effectively modulates, decrease, or reduces the levels of IKZF2 protein or degrades IKZF2 protein to a greater extent than any other protein, particularly any protein (transcription factor) from the Ikaros protein family (e.g., IKZF1, IKZF3, IKZF4, and IKZF5).

A “selective IKZF2 modulator”, “selective IKZF2 degrader”, or “selective IKZF2 compound” can be identified, for example, by comparing the ability of a compound to modulate IKZF2 protein levels to its ability to modulate levels of other members of the Ikaros protein family or other proteins. For example, a substance may be assayed for its ability to modulate IKZF2 protein levels, as well as IKZF1, IKZF3, IKZF4, IKZF5, and other proteins. In some embodiments, the selectivity can be identified by measuring the EC₅₀ of the compounds. In some embodiments, the selectivity can be identified by measuring the AC₅₀ of the compounds. In some embodiments, a selective IKZF2 degrader is identified by comparing the ability of a compound to degrade IKZF2 to its ability to degrade other members of the Ikaros protein family or other proteins.

In certain embodiments, the compounds of the application are IKZF2 degraders that exhibit at least 2-fold, 3-fold, 5-fold, 10-fold, 25-fold, 50-fold or 100-fold selectivity for the degradation of IKZF2 over other proteins (e.g., IKZF1, IKZF3, IKZF4, and IKZF5). In various embodiments, the compounds of the application exhibit up to 1000-fold selectivity for the degradation of IKZF2 over other proteins.

In certain embodiments, the compounds of the application exhibit at least 2-fold, 3-fold, 5-fold, 10-fold, 25-fold, 50-fold or 100-fold selectivity for the degradation of IKZF2 over the other members of the Ikaros protein family (e.g., IKZF1, IKZF3, IKZF4, and IKZF5). In various embodiments, the compounds of the application exhibit up to 1000-fold selectivity for the degradation of IKZF2 over the other members of the Ikaros protein family (e.g., IKZF1, IKZF3, IKZF4, and IKZF5).

In certain embodiments, the compounds of the application exhibit at least 2-fold, 3-fold, 5-fold, 10-fold, 25-fold, 50-fold or 100-fold selectivity for the degradation of IKZF2 over IKZF1. In various embodiments, the compounds of the application exhibit up to 1000-fold selectivity for the degradation of IKZF2 over IKZF1.

In certain embodiments, the compounds of the application exhibit at least 2-fold, 3-fold, 5-fold, 10-fold, 25-fold, 50-fold or 100-fold selectivity for the degradation of IKZF2 over IKZF3. In various embodiments, the compounds of the application exhibit up to 1000-fold selectivity for the degradation of IKZF2 over IKZF3.

In certain embodiments, the compounds of the application exhibit at least 2-fold, 3-fold, 5-fold, 10-fold, 25-fold, 50-fold or 100-fold selectivity for the degradation of IKZF2 over IKZF4. In various embodiments, the compounds of the application exhibit up to 1000-fold selectivity for the degradation of IKZF2 over IKZF4.

In certain embodiments, the compounds of the application exhibit at least 2-fold, 3-fold, 5-fold, 10-fold, 25-fold, 50-fold or 100-fold selectivity for the degradation of IKZF2 over IKZF5. In various embodiments, the compounds of the application exhibit up to 1000-fold selectivity for the degradation of IKZF2 over IKZF5.

In certain embodiments, the compounds of the application exhibit at least 2-fold, 3-fold, 5-fold, 10-fold, 25-fold, 50-fold or 100-fold selectivity for the degradation of IKZF2 and IKZF4 over the other members of the Ikaros protein family (e.g., IKZF1, IKZF3, and IKZF5). In various embodiments, the compounds of the application exhibit up to 1000-fold selectivity for the degradation of IKZF2 and IKZF4 over the other members of the Ikaros protein family (e.g., IKZF1, IKZF3, and IKZF5).

In certain embodiments, the compounds of the application exhibit at least 2-fold, 3-fold, 5-fold, 10-fold, 25-fold, 50-fold or 100-fold selectivity for the degradation of IKZF2 and IKZF4 over IKZF1. In various embodiments, the compounds of the application exhibit up to 1000-fold selectivity for the degradation of IKZF2 and IKZF4 over IKZF1.

In certain embodiments, the compounds of the application exhibit at least 2-fold, 3-fold, 5-fold, 10-fold, 25-fold, 50-fold or 100-fold selectivity for the degradation of IKZF2 and IKZF4 over IKZF3. In various embodiments, the compounds of the application exhibit up to 1000-fold selectivity for the degradation of IKZF2 and IKZF4 over IKZF3.

In certain embodiments, the compounds of the application exhibit at least 2-fold, 3-fold, 5-fold, 10-fold, 25-fold, 50-fold or 100-fold selectivity for the degradation of IKZF2 and IKZF4 over IKZF5. In various embodiments, the compounds of the application exhibit up to 1000-fold selectivity for the degradation of IKZF2 and IKZF4 over IKZF5.

In some embodiments, the degradation of IKZF2 is measured by AC₅₀.

Potency of can be determined by AC₅₀ value. A compound with a lower AC₅₀ value, as determined under substantially similar degradation conditions, is a more potent degrader relative to a compound with a higher AC₅₀ value. In some embodiments, the substantially similar conditions comprise determining degradation of protein levels in cells expressing the specific protein, or a fragment of any thereof.

The disclosure is directed to compounds as described herein and pharmaceutically acceptable salts, hydrates, solvates, prodrugs, stereoisomers, or tautomers thereof, and pharmaceutical compositions comprising one or more compounds as described herein, or pharmaceutically acceptable salts, hydrates, solvates, prodrugs, stereoisomers, or tautomers thereof.

### E. Methods of Synthesizing Compounds of Formula (I) or a Compound of Embodiment 17

The compounds of the present disclosure may be made by a variety of methods, including standard chemistry. Suitable synthetic routes are depicted in the Schemes given below.

The compounds of the present disclosure may be prepared by methods known in the art of organic synthesis as set forth in part by the following synthetic schemes. In the schemes described below, it is well understood that protecting groups for sensitive or reactive groups are employed where necessary in accordance with general principles or chemistry. Protecting groups are manipulated according to standard methods of organic synthesis (T. W. Greene and P. G. M. Wuts, “Protective Groups in Organic Synthesis”, Third edition, Wiley, New York 1999). These groups are removed at a convenient stage of the compound synthesis using methods that are readily apparent to those skilled in the art. The selection processes, as well as the reaction conditions and order of their execution, shall be consistent with the preparation of Compounds of Formula (I) or a compound of Embodiment 17.

Those skilled in the art will recognize if a stereocenter exists in the compounds of the present disclosure. Accordingly, the present disclosure includes both possible stereoisomers (unless specified in the synthesis) and includes not only racemic compounds but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-Interscience, 1994).

The compounds described herein may be made from commercially available starting materials or synthesized using known organic, inorganic, and/or enzymatic processes.

### Preparation of Compounds

The compounds of the present disclosure can be prepared in a number of ways well known to those skilled in the art of organic synthesis. By way of example, compounds of the present disclosure can be synthesized using the methods described below, together with synthetic methods known in the art of synthetic organic chemistry, or variations thereon as appreciated by those skilled in the art. Preferred methods include but are not limited to those methods described below.

Compounds of the present disclosure can be synthesized by following the steps outlined in General Schemes I to IV which comprise different sequences of assembling intermediates 1-a to 1-g, 2-a, 2-b, 2-c, 3-a, 4-a, and 4-b. Starting materials are either commercially available or made by known procedures in the reported literature or as illustrated.

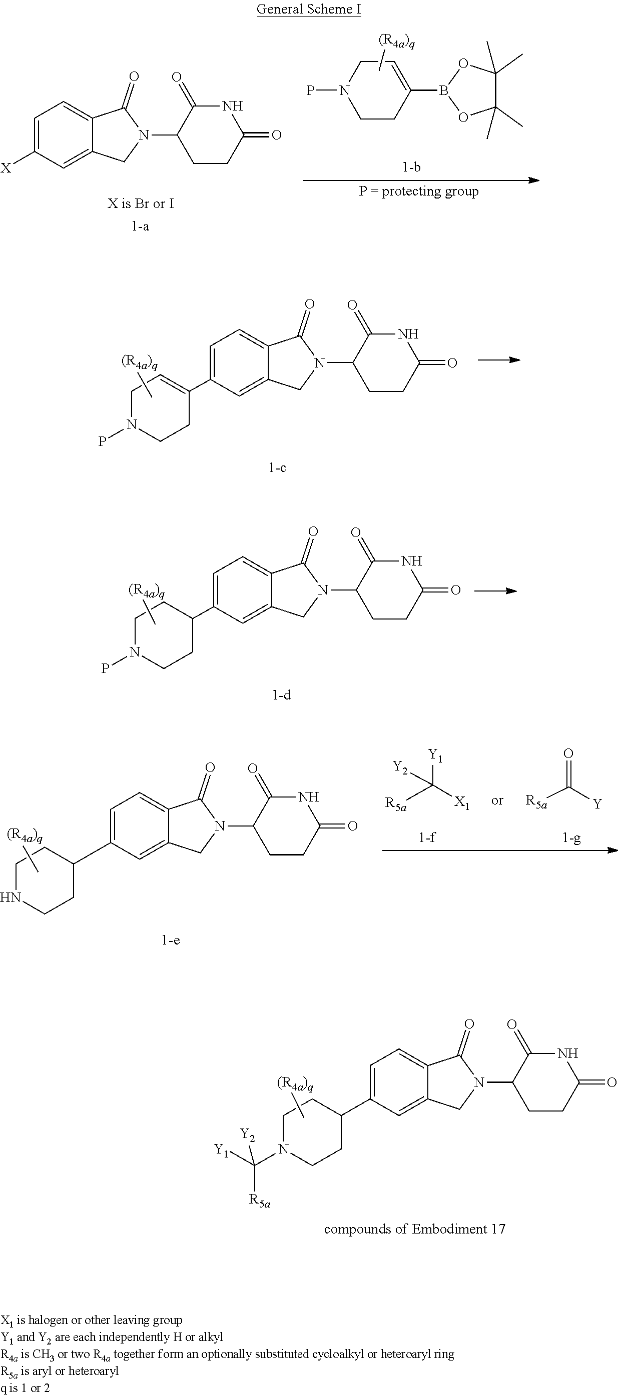

The general way of preparing compounds of Embodiment 17 (e.g., (I-1)-(I-18) and((I-24)—(I-38)), by using intermediates 1-a, 1-b, 1-c, 1-d, 1-e, 1-f, and 1-g is outlined in General Scheme I. Coupling of 1-a with boronic ester 1-b using a catalyst (e.g., Pd(dppf)Cl₂·DCM), and a base (e.g., cesium carbonate (Cs₂CO₃)), in a solvent (e.g., N,N-dimethylformamide (DMF)) at elevated temperature yields 1-c. Hydrogenation of 1-c in the presence of a suitable catalyst (e.g., Pd/C or PtO₂) in a solvent (e.g., DMF) and under an atmosphere of hydrogen gas provides 1-d.

Removal of the amine protecting group (e.g., tert-butyloxycarbonyl (Boc)) on intermediate 1-d can be accomplished using a strong acid such as trifluoroacetic acid (TFA) or hydrochloric acid (HCl) in a solvent (e.g., tetrahydrofuran (THF), 1,2-dichloroethane, dioxane or dichloromethane (DCM)) optionally at elevated temperature to provide I-e. Reductive amination of 1-e with aldehyde or ketone 1-g provides the desired product. Alternatively, compounds of Embodiment 17 where X₁ is CH and R₂ is a substituted alkyl can be obtained by alkylation of I-e with an alkyl halide 1-f in the presence of a base (e.g., triethyl amine (TEA), cesium carbonate (Cs₂CO₃), etc.), in a solvent (e.g., DCM, DMF, etc.), and optionally at elevated temperature.

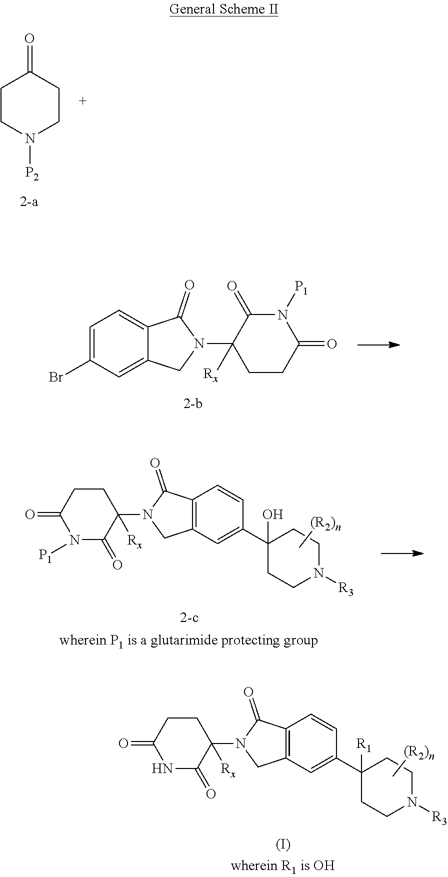

wherein Rₓ, R₂, R₃ and n are as defined herein above.

The general way of preparing compounds of Formula (I) wherein R₁ is OH by using intermediates 2-a, 2-b, and 2-c is outlined in General Scheme II. Alkylation of ketone 2-a with 2-b in the presence of a strong base (e.g., n-butyl lithium (n-BuLi), tert-butyl lithium (t-BuLi), sec-butyl lithium (s-BuLi)) in a solvent (e.g., tetrahydrofuran (THF), diethyl ether (Et₂O)), optionally at cold temperatures provides 2-c. Removal of the glutarimide protecting group (e.g., para-methoxybenzyl (PMB) or [2-(Trimethylsilyl)ethoxy]methyl acetal (SEM)) can be accomplished in the presence of strong acid (e.g., HCl or TFA) optionally in a solvent (e.g., THF, 1,2-dichloroethane, dioxane, or dichloromethane (DCM)) and optionally followed by treatment with a base (e.g., TEA) in a solvent and in the presence of N1,N2-dimethylethane-1,2-diamine (when P₁ is SEM) provides the desired compound of Formula (I) wherein R₁ is OH.

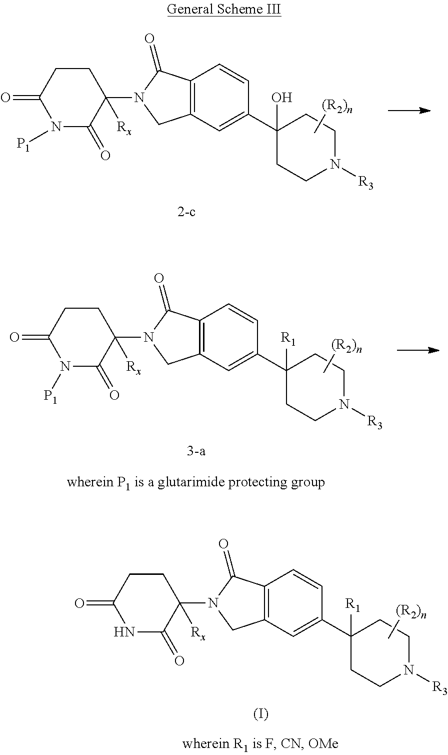

wherein Rₓ, R₂, R₃ and n are as defined herein above.

The general way of preparing compounds of Formula (I) wherein R₁ is F, CN or OMe by using intermediates 2-c and 3-a is outlined in General Scheme III. Treatment of 2-c with a fluorinating agent (e.g., diethylaminosulfur trifluoride (DAST)) in a solvent (e.g., DCM) optionally at cold temperatures followed by removal of the glutarimide protecting group as described above for General Scheme II provides the desired compound of Formula (I) wherein R₁ is F. Alternatively, treatment of 2-c with trimethylsilyl cyanide (TMSCN) and a Lewis Acid (e.g., zinc(II) iodide (ZnI₂), scandium(III) triflate (Sc(OTf)₃), tin(IV) chloride (SnCl₄), indium(III) chloride (InCl₃), or titanium montmorillonite) in a solvent (e.g., DCM) optionally at cold temperatures followed by removal of the glutarimide protecting group as described above for General Scheme II provides the desired compound of Formula (I) wherein R₁ is CN. Furthermore, compounds of Formula (I) wherein R₁ is OMe can be obtained by the treatment of 2-c with a methylating agent (e.g., diazomethane (CH₂N₂), trimethyloxonium tetrafluoroborate ([Me₃O]⁺[BF₄]⁻, methyl triflate (MeOTf)) in a solvent (e.g., DCM, MeCN, THF) optionally in the presence of base (N,N-diisopropylethylamine (i-Pr₂NEt), sodium hydride (NaH)) and optionally at cold temperatures followed by removal of the glutarimide protecting group.

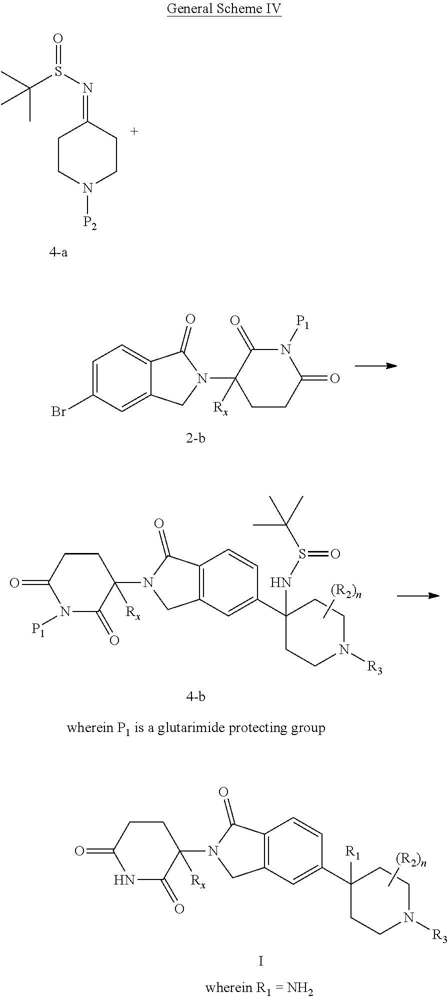

wherein Rₓ, R₂, R₃ and n are as defined herein above.

The general way of preparing compounds of Formula (I) wherein R₁ is NH₂ by using intermediates 4-a, 2-b, and 4-b is outlined in General Scheme IV. Alkylation of ketone 4-a with 2-b in the presence of a strong base (e.g., n-butyl lithium (n-BuLi), tert-butyl lithium (t-BuLi), sec-butyl lithium (s-BuLi)) in a solvent (e.g., tetrahydrofuran (THF), diethyl ether (Et₂O)), optionally at cold temperatures provides 4-b. Removal of the glutarimide protecting group (e.g., para-methoxybenzyl (PMB) or [2-(Trimethylsilyl)ethoxy]methyl acetal (SEM)) can be accomplished in the presence of strong acid (e.g., HCl or TFA) optionally in a solvent (e.g., THF, 1,2-dichloroethane, dioxane or dichloromethane (DCM)) and optionally followed by treatment with a base (e.g., TEA) in a solvent and in the presence of N1,N2-dimethylethane-1,2-diamine (when P₁ is SEM) provides the desired compound of Formula (I) wherein R₁ is NH₂.

A mixture of enantiomers, diastereomers, and cis/trans isomers resulting from the process described above can be separated into their single components by chiral salt technique, chromatography using normal phase, reverse phase or chiral column, depending on the nature of the separation.

Any resulting racemates of compounds of the present disclosure or of intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present disclosure into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid, or camphor-10-sulfonic acid. Racemic compounds of the present disclosure or racemic intermediates can also be resolved by chiral chromatography, e.g., high pressure liquid chromatography (HPLC) using a chiral adsorbent.

Any resulting mixtures of stereoisomers can be separated on the basis of the physicochemical differences of the constituents, into the pure or substantially pure geometric or optical isomers, diastereomers, racemates, for example, by chromatography and/or fractional crystallization.

It should be understood that in the description and formula shown above, the various groups Rₓ, R₂, R₃ and n and other variables are as defined above, except where otherwise indicated. Furthermore, for synthetic purposes, the compounds of General Schemes I to IV are merely representative with elected radicals to illustrate the general synthetic methodology of the Compounds of Formula (I) as defined herein.

### F. Methods of Using Compounds of Formula (I) or a Compound of Embodiment 16, 17, or 35

Another aspect of the disclosure relates to a method of treating, preventing, inhibiting, or eliminating a disease or disorder in a patient associated with or affected by modulation of IKZF2 protein levels. The method comprises administering to a patient in need of a treatment for diseases or disorders associated with modulation of IKZF2 protein levels an effective amount of a compound of Formula (I), or Embodiment 16, 17, or 35, or a pharmaceutically acceptable salt, hydrate, solvate, prodrug, stereoisomer, or tautomer thereof or a composition comprising a compound of Formula (I) or Embodiment 16, 17, or 35, or a pharmaceutically acceptable salt, hydrate, solvate, prodrug, stereoisomer, or tautomer thereof.

In another aspect, the disclosure relates to a method of treating, preventing, inhibiting, or eliminating a disease or disorder that is affected by the reduction of or decrease in IKZF2 protein levels. The method comprises administering to a patient in need of a treatment for diseases or disorders affected by the reduction of IKZF2 protein levels an effective amount of a compound of Formula (I) or Embodiment 16, 17, or 35, or a pharmaceutically acceptable salt, hydrate, solvate, prodrug, stereoisomer, or tautomer thereof or a composition comprising a compound of Formula (I) or Embodiment 16, 17, or 35, or a pharmaceutically acceptable salt, hydrate, solvate, prodrug, stereoisomer, or tautomer thereof.

Another aspect of the disclosure relates to the use of a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, in the manufacture of a medicament for the treatment, prevention, inhibition or elimination of a disease or disorder that is associated with or affected by the modulation of IKZF2 protein levels.

In another aspect, the disclosure relates to the use of a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, in the manufacture of a medicament for the treatment, prevention, inhibition or elimination of a disease or disorder that is affected by the reduction of or a decrease in IKZF2 protein levels.

Another aspect of the disclosure relates to a compound of Formula (I) or Embodiment 16, 17, or 35, or a pharmaceutically acceptable salt, hydrate, solvate, prodrug, stereoisomer, or tautomer thereof or a composition comprising a compound of Formula (I) or Embodiment 16, 17, or 35, or a pharmaceutically acceptable salt, hydrate, solvate, prodrug, stereoisomer, or tautomer thereof, for use in the manufacture of a medicament for treating, preventing, inhibiting, or eliminating a disease or disorder that is associated with or affected by the modulation of, the reduction of, or a decrease in IKZF2 protein levels.

In another aspect, the present disclosure is directed to a method of modulating, reducing, or decreasing IKZF2 protein levels. The method involves administering to a patient in need thereof an effective amount of a compound of Formula (I) or Embodiment 16, 17, or 35, or a pharmaceutically acceptable salt, hydrate, solvate, prodrug, stereoisomer, or tautomer thereof or a composition comprising a compound of Formula (I) or Embodiment 16, 17, or 35, or a pharmaceutically acceptable salt, hydrate, solvate, prodrug, stereoisomer, or tautomer thereof. In some embodiments, IKZF2 protein levels are modulated, reduced, or decreased through degradation of the IKZF2 protein. In other embodiments, IKZF2 protein levels are modulated, reduced, or decreased through degradation of the IKZF2 protein mediated by an E3 ligase.

Another aspect of the present disclosure relates to a method of treating, preventing, inhibiting, or eliminating a disease or disorder in a patient associated with the reduction of or decrease in IKZF2 protein levels, the method comprising administering to a patient in need thereof an effective amount of a compound of Formula (I) or Embodiment 16, 17, or 35, or a pharmaceutically acceptable salt, hydrate, solvate, prodrug, stereoisomer, or tautomer thereof or a composition comprising a compound of Formula (I) or Embodiment 16, 17, or 35, or a pharmaceutically acceptable salt, hydrate, solvate, prodrug, stereoisomer, or tautomer thereof.

The present disclosure also relates to the use of a degrader of IKZF2 for the preparation of a medicament used in the treatment, prevention, inhibition or elimination of a IKZF2-dependent disease or disorder, wherein the medicament comprises a compound of Formula (I) or Embodiment 16, 17, or 35, or a pharmaceutically acceptable salt, hydrate, solvate, prodrug, stereoisomer, or tautomer thereof or a composition comprising a Compound of Formula (I) or Embodiment 16, 17, or 35, or a pharmaceutically acceptable salt, hydrate, solvate, prodrug, stereoisomer, or tautomer thereof.

In another aspect, the present disclosure relates to a method for treating, preventing, inhibiting, or eliminating a IKZF2-dependent disease or disorder, wherein the medicament comprises a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof.

In another aspect, the present disclosure relates to a method for the manufacture of a medicament for treating, preventing, inhibiting, or eliminating a IKZF2-dependent disease or disorder mediated, wherein the medicament comprises a compound of Formula (I) or Embodiment 16, 17, or 35, or a pharmaceutically acceptable salt, hydrate, solvate, prodrug, stereoisomer, or tautomer thereof or a composition comprising a compound of Formula (I) or Embodiment 16, 17, or 35, or a pharmaceutically acceptable salt, hydrate, solvate, prodrug, stereoisomer, or tautomer thereof.

Another aspect of the present disclosure relates to a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, for use in the manufacture of a medicament for treating a disease or disorder associated with the modulation of, the reduction of, or a decrease in IKZF2 protein levels. In some embodiments, IKZF2 levels are modulated through degradation of the IKZF2 protein. In some embodiments, IKZF2 protein levels are modulated through degradation of the IKZF2 protein mediated by an E3 ligase.

Another aspect of the present disclosure relates to a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, for use in treating a disease associated with the modulation of, the reduction of, or a decrease in IKZF2 protein levels. In some embodiments, IKZF2 levels are modulated, reduced, or decreased through degradation of the IKZF2 protein. In some embodiments, IKZF2 protein levels are modulated, reduced, or decreased through degradation of the IKZF2 protein mediated by an E3 ligase.

In another aspect, the present disclosure relates to the use of a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, in the treatment of a disease associated with the modulation of, the reduction of, or a decrease in IKZF2 protein levels. In some embodiments, IKZF2 protein levels are modulated, reduced, or decreased through degradation of the IKZF2 protein. In some embodiments, IKZF2 protein levels are modulated, reduced, or decreased through degradation of the IKZF2 protein mediated by an E3 ligase.

In another aspect, the present disclosure relates to a method of inhibiting IKZF2 activity through degradation of IKZF2. In some embodiments, IKZF2 protein degradation is mediated by an E3 ligase.

Another aspect of the disclosure relates to the use of a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, for inhibiting IKZF2 activity through degradation of IKZF2. In some embodiments, IKZF2 protein degradation is mediated by an E3 ligase.

In another aspect, the present disclosure relates to a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, for use in the inhibition of IKZF2 activity through degradation of IKZF2. In some embodiments, IKZF2 protein degradation is mediated by an E3 ligase.

Another aspect of the disclosure relates to a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, for use in the manufacture of a medicament for inhibiting IKZF2 activity through degradation of IKZF2. In some embodiments, IKZF2 protein degradation is mediated by an E3 ligase.

In another aspect, the present disclosure relates to a method of inhibiting IKZF2 and IKZF4 activity through degradation of IKZF2 and IKZF4. In some embodiments, IKZF2 and IKZF4 protein degradation is mediated by an E3 ligase.

Another aspect of the disclosure relates to the use of a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, for inhibiting IKZF2 and IKZF4 activity through degradation of IKZF2 and IKZF4. In some embodiments, IKZF2 and IKZF4 protein degradation is mediated by an E3 ligase.

In another aspect, the present disclosure relates to a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, for use in the inhibition of IKZF2 and IKZF4 activity through degradation of IKZF2 and IKZF4. In some embodiments, IKZF2 and IKZF4 protein degradation is mediated by an E3 ligase.

Another aspect of the disclosure relates to a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, for use in the manufacture of a medicament for inhibiting IKZF2 and IKZF4 activity through degradation of IKZF2 and IKZF4. In some embodiments, IKZF2 and IKZF4 protein degradation is mediated by an E3 ligase.

Another aspect of the disclosure relates to a method of treating, preventing, inhibiting, or eliminating a disease or disorder associated with the modulation of, the reduction of, or a decrease in IKZF2 and IKZF4 protein levels. The method comprises administering to a patient in need thereof an effective amount of a compound of Formula (I) or Embodiment 16, 17, or 35, or a pharmaceutically acceptable salt, hydrate, solvate, prodrug, stereoisomer, or tautomer thereof or a composition comprising a compound of Formula (I) or Embodiment 16, 17, or 35, or a pharmaceutically acceptable salt, hydrate, solvate, prodrug, stereoisomer, or tautomer thereof.

In another aspect, the present disclosure is directed to a method of modulating, reducing, or decreasing IKZF2 and IKZF4 protein levels. The method involves administering to a patient in need thereof an effective amount of a compound of Formula (I) or Embodiment 16, 17, or 35, or a pharmaceutically acceptable salt, hydrate, solvate, prodrug, stereoisomer, or tautomer thereof or a composition comprising a compound of Formula (I) or Embodiment 16, 17, or 35, or a pharmaceutically acceptable salt, hydrate, solvate, prodrug, stereoisomer, or tautomer thereof. In some embodiments, IKZF2 and IKZF4 protein levels are modulated, reduced, or decreased through degradation of the IKZF2 and IKZF4 proteins. In other embodiments, IKZF2 and IKZF4 protein levels are modulated through degradation of the IKZF2 and IKZF4 proteins mediated by an E3 ligase.

Another aspect of the disclosure relates to a method of treating, preventing, inhibiting, or eliminating a disease or disorder associated with modulation of, reduction of, or a decrease in IKZF4 protein levels. The method comprises administering to a patient in need thereof an effective amount of a compound of Formula (I) or Embodiment 16, 17, or 35, or a pharmaceutically acceptable salt, hydrate, solvate, prodrug, stereoisomer, or tautomer thereof or a composition comprising a compound of Formula (I) or Embodiment 16, 17, or 35, or a pharmaceutically acceptable salt, hydrate, solvate, prodrug, stereoisomer, or tautomer thereof. In some embodiments, IKZF4 protein levels are modulated, reduced, or decreased through degradation of the IKZF4 proteins. In some embodiments, IKZF4 protein levels are modulated, reduced, or decreased through degradation of the IKZF4 protein mediated by an E3 ligase.

In another aspect, the present disclosure is directed to a method of modulating, reducing, or decreasing IKZF4 protein levels. The method involves administering to a patient in need thereof an effective amount of a compound of Formula (I) or Embodiment 16, 17, or 35, or a pharmaceutically acceptable salt, hydrate, solvate, prodrug, stereoisomer, or tautomer thereof or a composition comprising a compound of Formula (I) or Embodiment 16, 17, or 35, or a pharmaceutically acceptable salt, hydrate, solvate, prodrug, stereoisomer, or tautomer thereof. In some embodiments, IKZF4 protein levels are modulated, reduced, or decreased through degradation of the IKZF4 proteins. In other embodiments, IKZF4 protein levels are modulated, reduced, or decreased through degradation of the IKZF4 protein mediated by an E3 ligase.

Another aspect of the disclosure relates to the use of a compound of Formula (I) or Embodiment 16, 17, or 35, or a pharmaceutically acceptable salt, hydrate, solvate, prodrug, stereoisomer, or tautomer thereof or a composition comprising a compound of Formula (I) or Embodiment 16, 17, or 35, or a pharmaceutically acceptable salt, hydrate, solvate, prodrug, stereoisomer, or tautomer thereof, for treating, preventing, inhibiting, or eliminating a disease or disorder associated with modulation of, reduction of, or a decrease in IKZF4 protein levels. In some embodiments, IKZF4 protein levels are modulated, reduced, or decreased through degradation of the IKZF4 proteins. In some embodiments, IKZF4 protein levels are modulated, reduced, or decreased through degradation of the IKZF4 protein mediated by an E3 ligase.

Another aspect of the disclosure relates to a compound of Formula (I) or Embodiment 16, 17, or 35, or a pharmaceutically acceptable salt, hydrate, solvate, prodrug, stereoisomer, or tautomer thereof or a composition comprising a compound of Formula (I) or Embodiment 16, 17, or 35, or a pharmaceutically acceptable salt, hydrate, solvate, prodrug, stereoisomer, or tautomer thereof, for use in treating, preventing, inhibiting, or eliminating a disease or disorder associated with modulation of, reduction of, or a decrease in IKZF4 protein levels. In some embodiments, IKZF4 protein levels are modulated, reduced, or decreased through degradation of the IKZF4 proteins. In some embodiments, IKZF4 protein levels are modulated, reduced, or decreased through degradation of the IKZF4 protein mediated by an E3 ligase.

In another aspect, the present disclosure is directed to a compound of Formula (I) or Embodiment 16, 17, or 35, or a pharmaceutically acceptable salt, hydrate, solvate, prodrug, stereoisomer, or tautomer thereof or a composition comprising a compound of Formula (I) or Embodiment 16, 17, or 35, or a pharmaceutically acceptable salt, hydrate, solvate, prodrug, stereoisomer, or tautomer thereof, for use in the manufacture of a medicament for treating, preventing, inhibiting, or eliminating a disease or disorder associated with modulation of, reduction of, or a decrease in IKZF4 protein levels. In some embodiments, IKZF4 protein levels are modulated, reduced, or decreased through degradation of the IKZF4 proteins. In some embodiments, IKZF4 protein levels are modulated, reduced, or decreased through degradation of the IKZF4 protein mediated by an E3 ligase.

Another aspect of the disclosure relates to a method of treating, preventing, inhibiting, or eliminating a disease or disorder associated with a decrease in IKZF2 and IKZF4 protein levels. The method comprises administering to a patient in need of a treatment for diseases or disorders associated with a decrease of IKZF2 and IKZF4 protein levels an effective amount of a compound of Formula (I) or Embodiment 16, 17, or 35, or a pharmaceutically acceptable salt, hydrate, solvate, prodrug, stereoisomer, or tautomer thereof or a composition comprising a compound of Formula (I) or Embodiment 16, 17, or 35, or a pharmaceutically acceptable salt, hydrate, solvate, prodrug, stereoisomer, or tautomer thereof.

The present disclosure also relates to the use of a modulator of IKZF2 and IKZF4 protein levels for the preparation of a medicament used in the treatment, prevention, inhibition or elimination of a IKZF2 and IKZF4-dependent disease or disorder, wherein the medicament comprises a compound of Formula (I) or Embodiment 16, 17, or 35, or a pharmaceutically acceptable salt, hydrate, solvate, prodrug, stereoisomer, or tautomer thereof or a composition comprising a compound of Formula (I) or Embodiment 16, 17, or 35, or a pharmaceutically acceptable salt, hydrate, solvate, prodrug, stereoisomer, or tautomer thereof. In another aspect, the present disclosure relates to a method for the manufacture of a medicament for treating, preventing, inhibiting, or eliminating a IKZF2 and IKZF4-dependent disease or disorder, wherein the medicament comprises a compound of Formula (I) or Embodiment 16, 17, or 35, or a pharmaceutically acceptable salt, hydrate, solvate, prodrug, stereoisomer, or tautomer thereof or a composition comprising a compound of Formula (I) or Embodiment 16, 17, or 35, or a pharmaceutically acceptable salt, hydrate, solvate, prodrug, stereoisomer, or tautomer thereof.

Another aspect of the present disclosure relates to a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, for use in the manufacture of a medicament for treating a disease associated with the modulation of, the reduction of, or a decrease in IKZF2 and IKZF4 protein levels. In some embodiments, IKZF2 and IKZF4 protein levels are modulated, reduced, or decreased through degradation of the IKZF2 and IKZF4 proteins. In other embodiments, IKZF2 and IKZF4 protein levels are modulated, reduced, or decreased through degradation of the IKZF2 and IKZF4 proteins mediated by an E3 ligase.

In another aspect, the present disclosure relates to a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, for use in treating a disease associated with the modulation of, the reduction of, or a decrease in IKZF2 and IKZF4 protein levels. In some embodiments, IKZF2 and IKZF4 protein levels are modulated, reduced, or decreased through degradation of the IKZF2 and IKZF4 proteins. In other embodiments, IKZF2 and IKZF4 protein levels are modulated, reduced, or decreased through degradation of the IKZF2 and IKZF4 proteins mediated by an E3 ligase.

In another aspect, the present disclosure relates to the use of a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, in the treatment of a disease associated with the modulation of, the reduction of, or a decrease in IKZF2 and IKZF4 protein levels. In some embodiments, IKZF2 and IKZF4 protein levels are modulated, reduced, or decreased through degradation of the IKZF2 and IKZF4 proteins. In other embodiments, IKZF2 and IKZF4 protein levels are modulated, reduced, or decreased through degradation of the IKZF2 and IKZF4 proteins mediated by an E3 ligase.

Another aspect of the disclosure relates to a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, for use in the treatment of an IKZF2-dependent disease or disorder by reducing or decreasing IKZF2 protein levels, wherein reduction or decrease of IKZF2 protein levels treats the IKZF2-dependent disease or disorder.

In another aspect, the present disclosure the use of a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, in the treatment of an IKZF2-dependent disease or disorder by reducing or decreasing IKZF2 protein levels wherein reduction of or decrease in IKZF2 protein levels treats the IKZF2-dependent disease or disorder.

In another aspect, the present disclosure the use of a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, in the manufacture of a medicament for treating an IKZF2-dependent disease or disorder by reducing or decreasing IKZF2 protein levels wherein reduction of or decrease in IKZF2 protein levels treats the IKZF2-dependent disease or disorder.

Another aspect of the disclosure relates to a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, for use in the treatment of an IKZF2 and IKZF4-dependent disease or disorder by reducing or decreasing IKZF2 and IKZF4 protein levels wherein the reduction of or decrease in IKZF2 and IKZF4 protein levels treats the IKZF2 and IKZF4-dependent disease or disorder.

In another aspect, the present disclosure the use of a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, in the treatment of an IKZF2 and IKZF4-dependent disease or disorder by reducing or decreasing IKZF2 and IKZF4 protein levels wherein the reduction of or decrease in IKZF2 and IKZF4 protein levels treats the IKZF2 and IKZF4-dependent disease or disorder.

In another aspect, the present disclosure the use of a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, in the manufacture of a medicament for treating an IKZF2 and IKZF4-dependent disease or disorder by reducing or decreasing IKZF2 and IKZF4 protein levels wherein the reduction of or decrease in IKZF2 and IKZF4 protein levels treats the IKZF2 and IKZF4-dependent disease or disorder.

Another aspect of the disclosure relates to a method of treating cancer. The method comprises administering to a patient in need thereof an effective amount of a compound of Formula (I) or Embodiment 16, 17, or 35, or a pharmaceutically acceptable salt, hydrate, solvate, prodrug, stereoisomer, or tautomer thereof or a composition comprising a compound of Formula (I) or Embodiment 16, 17, or 35, or a pharmaceutically acceptable salt, hydrate, solvate, prodrug, stereoisomer, or tautomer thereof.

In another aspect, the present disclosure relates to the use of a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, in the treatment of treating cancer.

Another aspect of the disclosure relates to a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, for use in the manufacture of a medicament for treating cancer.

In another aspect, the present disclosure relates to a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, for use in the treatment of cancer.

Another aspect of the disclosure relates to a method of treating an IKZF2-dependent cancer. The method comprises administering to a patient in need thereof an effective amount of a compound of Formula (I) or Embodiment 16, 17, or 35, or a pharmaceutically acceptable salt, hydrate, solvate, prodrug, stereoisomer, or tautomer thereof or a composition comprising a compound of Formula (I) or Embodiment 16, 17, or 35, or a pharmaceutically acceptable salt, hydrate, solvate, prodrug, stereoisomer, or tautomer thereof.

In another aspect, the present disclosure relates to the use of a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, in the treatment of treating an IKZF2-dependent cancer.

Another aspect of the disclosure relates to a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, for use in the manufacture of a medicament for treating an IKZF2-dependent cancer.

In another aspect, the present disclosure relates to a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, for use in the treatment of an IKZF2-dependent cancer.

Another aspect of the disclosure relates to a method of treating an IKZF2-dependent and IKZF4-dependent cancer. The method comprises administering to a patient in need thereof an effective amount of a compound of Formula (I) or Embodiment 16, 17, or 35, or a pharmaceutically acceptable salt, hydrate, solvate, prodrug, stereoisomer, or tautomer thereof or a composition comprising a compound of Formula (I) or Embodiment 16, 17, or 35, or a pharmaceutically acceptable salt, hydrate, solvate, prodrug, stereoisomer, or tautomer thereof.

In another aspect, the present disclosure relates to the use of a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, in the treatment of treating an IKZF2-dependent and IKZF4-dependent cancer.

Another aspect of the disclosure relates to a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, for use in the manufacture of a medicament for treating an IKZF2-dependent and IKZF4-dependent cancer.

In another aspect, the present disclosure relates to a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, for use in the treatment of an IKZF2-dependent and IKZF4-dependent cancer.

Another aspect of the disclosure relates to a method of treating a cancer affected by the modulation of, the reduction of, or a decrease in IKZF2 protein levels. The method comprises administering to a patient in need thereof an effective amount of a compound of Formula (I) or Embodiment 16, 17, or 35, or a pharmaceutically acceptable salt, hydrate, solvate, prodrug, stereoisomer, or tautomer thereof or a composition comprising a compound of Formula (I) or Embodiment 16, 17, or 35, or a pharmaceutically acceptable salt, hydrate, solvate, prodrug, stereoisomer, or tautomer thereof.

In another aspect, the present disclosure relates to the use of a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, in the treatment of treating a cancer affected by the modulation of, the reduction of, or a decrease in IKZF2 protein levels

Another aspect of the disclosure relates to a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, for use in the manufacture of a medicament for treating a cancer affected by the modulation of, the reduction of, or a decrease in IKZF2 protein levels.

In another aspect, the present disclosure relates to a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, for use in the treatment of a cancer affected by the modulation of, the reduction of, or a decrease in IKZF2 protein levels.

Another aspect of the disclosure relates to a method of treating a cancer affected by the modulation of, the reduction of, or a decrease in IKZF2 and IKZF4 protein levels. The method comprises administering to a patient in need thereof an effective amount of a compound of Formula (I) or Embodiment 16, 17, or 35, or a pharmaceutically acceptable salt, hydrate, solvate, prodrug, stereoisomer, or tautomer thereof or a composition comprising a compound of Formula (I) or Embodiment 16, 17, or 35, or a pharmaceutically acceptable salt, hydrate, solvate, prodrug, stereoisomer, or tautomer thereof.

In another aspect, the present disclosure relates to the use of a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, in the treatment of treating a cancer affected by the modulation of, the reduction of, or a decrease in IKZF2 and IKZF4 protein levels.

Another aspect of the disclosure relates to a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, for use in the manufacture of a medicament for treating a cancer affected by the modulation of, the reduction of, or a decrease in IKZF2 and IKZF4 protein levels.

In another aspect, the present disclosure relates to a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, for use in the treatment of a cancer affected by the modulation of, the reduction of, or a decrease in IKZF2 and IKZF4 protein levels.

Another aspect of the disclosure relates to a method of degrading IKZF2. The method comprises administering to a patient in need thereof an effective amount of a compound of Formula (I) or Embodiment 16, 17, or 35, or a pharmaceutically acceptable salt, hydrate, solvate, prodrug, stereoisomer, or tautomer thereof or a composition comprising a compound of Formula (I) or Embodiment 16, 17, or 35, or a pharmaceutically acceptable salt, hydrate, solvate, prodrug, stereoisomer, or tautomer thereof. In some embodiments, IKZF2 protein degradation is mediated by an E3 ligase.

In another aspect, the present disclosure relates to the use of a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, for degrading IKZF2. In some embodiments, IKZF2 protein degradation is mediated by an E3 ligase.

Another aspect of the disclosure relates to a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, for use in the degradation IKZF2. In some embodiments, IKZF2 protein degradation is mediated by an E3 ligase.

In another aspect, the present disclosure relates to a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, for use in the manufacture of a medicament for degrading IKZF2. In some embodiments, IKZF2 protein degradation is mediated by an E3 ligase.

In another aspect, the present disclosure relates to a method of modulating IKZF2 protein levels through degradation of IKZF2. The method comprises administering to a patient in need thereof an effective amount of a compound of Formula (I) or Embodiment 16, 17, or 35, or a pharmaceutically acceptable salt, hydrate, solvate, prodrug, stereoisomer, or tautomer thereof or a composition comprising a compound of Formula (I) or Embodiment 16, 17, or 35, or a pharmaceutically acceptable salt, hydrate, solvate, prodrug, stereoisomer, or tautomer thereof. In some embodiments, IKZF2 protein degradation is mediated by an E3 ligase.

Another aspect of the disclosure relates to the use of a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, for modulating IKZF2 protein levels through degradation of IKZF2. In some embodiments, IKZF2 protein degradation is mediated by an E3 ligase.

In another aspect, the present disclosure relates to a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, for use in the modulation IKZF2 protein levels through degradation of IKZF2. In some embodiments, IKZF2 protein degradation is mediated by an E3 ligase.

Another aspect of the disclosure relates to a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, for use in the manufacture of a medicament for modulating IKZF2 protein levels through degradation of IKZF2. In some embodiments, IKZF2 protein degradation is mediated by an E3 ligase.

Another aspect of the disclosure relates to a method of treating an IKZF2-dependent disease or disorder in a patient in need thereof by modulating IKZF2 protein levels through the degradation of IKZF2. In some embodiments, IKZF2 protein degradation is mediated by an E3 ligase.

In another aspect, the present disclosure relates to the use of a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, for treating an IKZF2-dependent disease or disorder in a patient in need thereof by modulating IKZF2 protein levels through the degradation of IKZF2. In some embodiments, IKZF2 protein degradation is mediated by an E3 ligase.

Another aspect of the disclosure relates to a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, for use in treating an IKZF2-dependent disease or disorder in a patient in need thereof, by modulating IKZF2 protein levels through the degradation of IKZF2. In some embodiments, IKZF2 protein degradation is mediated by an E3 ligase.

In another aspect, the present disclosure relates to a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, for use in the manufacture of a medicament for treating an IKZF2-dependent disease or disorder in a patient in need thereof by modulating IKZF2 protein levels through the degradation of IKZF2. In some embodiments, IKZF2 protein degradation is mediated by an E3 ligase.

Another aspect of the disclosure relates to a method of reducing the proliferation of a cell, the method comprising contacting the cell with a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, that reduces IKZF2 protein levels. In some embodiments, IKZF2 protein levels are reduced through degradation of the IKZF2 protein. In some embodiments, IKZF2 protein levels are reduced through degradation of the IKZF2 protein mediated by an E3 ligase.

In another aspect, the present disclosure relates to the use a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, for reducing the proliferation of a cell by reducing IKZF2 protein levels. In some embodiments, IKZF2 protein levels are reduced through degradation of the IKZF2 protein. In some embodiments, IKZF2 protein levels are reduced through degradation of the IKZF2 protein mediated by an E3 ligase.

Another aspect of the disclosure relates to a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, for use in reducing the proliferation of a cell by IKZF 2 protein levels. In some embodiments, IKZF2 protein levels are reduced through degradation of the IKZF2 protein. In some embodiments, IKZF2 protein levels are reduced through degradation of the IKZF2 protein mediated by an E3 ligase.

In another aspect, the present disclosure relates to a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, for use in the manufacture of a medicament for reducing the proliferation of a cell by reducing IKZF2 protein levels. In some embodiments, IKZF2 protein levels are reduced through degradation of the IKZF2 protein. In some embodiments, IKZF2 protein levels are reduced through degradation of the IKZF2 protein mediated by an E3 ligase.

In another aspect, the disclosure relates to a method of treating, preventing, inhibiting, or eliminating a disease or disorder that is affected by the modulation of, the reduction of, or a decrease in IKZF2 and IKZF4 protein levels. The method comprises administering to a patient in need thereof an effective amount of a compound of Formula (I) or Embodiment 16, 17, or 35, or a pharmaceutically acceptable salt, hydrate, solvate, prodrug, stereoisomer, or tautomer thereof or a composition comprising a compound of Formula (I) or Embodiment 16, 17, or 35, or a pharmaceutically acceptable salt, hydrate, solvate, prodrug, stereoisomer, or tautomer thereof.

In another aspect, the disclosure relates to the use of a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, in the manufacture of a medicament for the treatment, prevention, inhibition or elimination of a disease or disorder that is affected by the modulation of IKZF2 and IKZF4 protein levels.

Another aspect of the disclosure relates to a compound of Formula (I) or Embodiment 16, 17, or 35, or a pharmaceutically acceptable salt, hydrate, solvate, prodrug, stereoisomer, or tautomer thereof or a composition comprising a compound of Formula (I) or Embodiment 16, 17, or 35, or a pharmaceutically acceptable salt, hydrate, solvate, prodrug, stereoisomer, or tautomer thereof, for use in the manufacture of a medicament for treating, preventing, inhibiting, or eliminating a disease or disorder that is affected by the modulation of, the reduction of, or a decrease in IKZF2 and IKZF4 protein levels.

In another aspect, the disclosure relates to the use a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, in the manufacture of a medicament for the treatment, prevention, inhibition or elimination of a disease or disorder that is affected by the reduction of or a decrease in IKZF2 and IKZF4 protein levels.

Another aspect of the disclosure relates to a compound of Formula (I) or Embodiment 16, 17, or 35, or a pharmaceutically acceptable salt, hydrate, solvate, prodrug, stereoisomer, or tautomer thereof or a composition comprising a compound of Formula (I) or Embodiment 16, 17, or 35, or a pharmaceutically acceptable salt, hydrate, solvate, prodrug, stereoisomer, or tautomer thereof, for use in the manufacture of a medicament for treating, preventing, inhibiting, or eliminating a disease or disorder that is affected by the reduction of or a decrease in IKZF2 and IKZF4 protein levels.

Another aspect of the disclosure relates to a method of degrading IKZF2 and IKZF4. The method comprises administering to a patient in need thereof an effective amount of a compound of Formula (I) or Embodiment 16, 17, or 35, or a pharmaceutically acceptable salt, hydrate, solvate, prodrug, stereoisomer, or tautomer thereof or a composition comprising a compound of Formula (I) or Embodiment 16, 17, or 35, or a pharmaceutically acceptable salt, hydrate, solvate, prodrug, stereoisomer, or tautomer thereof. In some embodiments, IKZF2 and IKZF4 protein degradation is mediated by an E3 ligase.

In another aspect, the present disclosure relates to the use of a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, for degrading IKZF2 and IKZF4. In some embodiments, IKZF2 and IKZF4 protein degradation is mediated by an E3 ligase.

Another aspect of the disclosure relates to a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, for use in the degradation IKZF2 and IKZF4. In some embodiments, IKZF2 and IKZF4 protein degradation is mediated by an E3 ligase.

In another aspect, the present disclosure relates to a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, for use in the manufacture of a medicament for degrading IKZF2 and IKZF4. In some embodiments, IKZF2 and IKZF4 protein degradation is mediated by an E3 ligase.

In another aspect, the present disclosure relates to a method of modulating IKZF2 and IKZF4 protein levels through degradation of IKZF2 and IKZF4. In some embodiments, IKZF2 and IKZF4 protein degradation is mediated by an E3 ligase.

Another aspect of the disclosure relates to the use of a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, for modulating IKZF2 and IKZF4 protein levels through degradation of IKZF2 and IKZF4. In some embodiments, IKZF2 and IKZF4 protein degradation is mediated by an E3 ligase.

In another aspect, the present disclosure relates to a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, for use in the modulation of IKZF2 and IKZF4 protein levels through degradation of IKZF2 and IKZF4. In some embodiments, IKZF2 and IKZF4 protein degradation is mediated by an E3 ligase.

Another aspect of the disclosure relates to a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, for use in the manufacture of a medicament for modulating IKZF2 and IKZF4 protein levels through degradation of IKZF2 and IKZF4. In some embodiments, IKZF2 and IKZF4 protein degradation is mediated by an E3 ligase.

Another aspect of the disclosure relates to a method of treating an IKZF2-dependent and IKZF4-dependent disease or disorder in a patient in need thereof by modulating IKZF2 and IKZF4 protein levels through the degradation of IKZF2 and IKZF4. In some embodiments, IKZF2 and IKZF4 protein degradation is mediated by an E3 ligase.

In another aspect, the present disclosure relates to the use of a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, for treating an IKZF2-dependent and IKZF4-dependent disease or disorder in a patient in need thereof by modulating IKZF2 and IKZF4 protein levels through the degradation of IKZF2 and IKZF4. In some embodiments, IKZF2 and IKZF4 protein degradation is mediated by an E3 ligase.

Another aspect of the disclosure relates to a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, for use in treating an IKZF2-dependent and IKZF4-dependent disease or disorder in a patient in need thereof by modulating IKZF2 and IKZF4 protein levels through the degradation of IKZF2 and IKZF4. In some embodiments, IKZF2 and IKZF4 protein degradation is mediated by an E3 ligase.

In another aspect, the present disclosure relates to a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, for use in the manufacture of a medicament for treating an IKZF2-dependent or IKZF4-dependent disease or disorder in a patient in need thereof by modulating IKZF2 and IKZF4 protein levels through the degradation of IKZF2 and IKZF4. In some embodiments, IKZF2 and IKZF4 protein degradation is mediated by an E3 ligase.

Another aspect of the disclosure relates to a method of reducing the proliferation of a cell, the method comprising contacting the cell with a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, and reducing IKZF2 and IKZF4 protein levels. In some embodiments, IKZF2 and IKZF4 protein levels are reduced through degradation of the IKZF2 and IKZF4 proteins. In other embodiments, IKZF2 and IKZF4 protein levels are reduced through degradation of the IKZF2 and IKZF4 proteins mediated by an E3 ligase.

In another aspect, the present disclosure relates to the use a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, for reducing the proliferation of a cell by reducing IKZF2 and IKZF4 protein levels. In some embodiments, IKZF2 and IKZF4 protein levels are reduced through degradation of the IKZF2 and IKZF4 proteins. In other embodiments, IKZF2 and IKZF4 protein levels are reduced through degradation of the IKZF2 and IKZF4 proteins mediated by an E3 ligase.

Another aspect of the disclosure relates to a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, for use in reducing the proliferation of a cell by reducing IKZF2 and IKZF4 protein levels. In some embodiments, IKZF2 and IKZF4 protein levels are reduced through degradation of the IKZF2 and IKZF4 proteins. In other embodiments, IKZF2 and IKZF4 protein levels are reduced through degradation of the IKZF2 and IKZF4 proteins mediated by an E3 ligase.

In another aspect, the present disclosure relates to a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, for use in the manufacture of a medicament for reducing the proliferation of a cell by reducing IKZF2 and IKZF4 protein levels. In some embodiments, IKZF2 and IKZF4 protein levels are reduced through degradation of the IKZF2 and IKZF4 proteins. In other embodiments, IKZF2 and IKZF4 protein levels are reduced through degradation of the IKZF2 and IKZF4 proteins mediated by an E3 ligase.

In another aspect, the present disclosure relates to a method for treating an IKZF2-dependent disease or disorder. The method comprises the step of administering to a subject in need thereof a therapeutically effective amount of a compound of Formula (I) or Embodiment 16, 17, or 35, or a pharmaceutically acceptable salt, hydrate, solvate, prodrug, stereoisomer, or tautomer thereof or a composition comprising a compound of Formula (I) or Embodiment 16, 17, or 35, or a pharmaceutically acceptable salt, hydrate, solvate, prodrug, stereoisomer, or tautomer thereof.

Another aspect of the disclosure relates to a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, for use in the treatment of an IKZF2-dependent disease or disorder.

In another aspect, the present disclosure relates to the use of a compound of Formula (I) or Embodiment 16, 17, or 35, or a pharmaceutically acceptable salt, hydrate, solvate, prodrug, stereoisomer, or tautomer thereof, in the manufacture of a medicament for treating an IKZF2-dependent disease or disorder.

Another aspect of the disclosure relates to a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, for use in the manufacture of a medicament for treating an IKZF2-dependent disease or disorder.

In another aspect, the present disclosure relates to a method for treating an IKZF2-dependent and IKZF4-dependent disease or disorder. The method comprises the step of administering to a subject in need thereof a therapeutically effective amount of a compound of Formula (I) or Embodiment 16, 17, or 35, or a pharmaceutically acceptable salt, hydrate, solvate, prodrug, stereoisomer, or tautomer thereof or a composition comprising a compound of Formula (I) or Embodiment 16, 17, or 35, or a pharmaceutically acceptable salt, hydrate, solvate, prodrug, stereoisomer, or tautomer thereof.

Another aspect of the disclosure relates to a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, for use in the treatment of an IKZF2-dependent and IKZF4-dependent disease or disorder.

In another aspect, the present disclosure relates to the use of a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, in the manufacture of a medicament for treating an IKZF2-dependent and IKZF4-dependent disease or disorder.

Another aspect of the disclosure relates to a compound of Formula (I) or Embodiment 16, 17, or 35, or a pharmaceutically acceptable salt, hydrate, solvate, prodrug, stereoisomer, or tautomer thereof, or a composition comprising a compound of Formula (I) or Embodiment 16, 17, or 35, or a pharmaceutically acceptable salt, hydrate, solvate, prodrug, stereoisomer, or tautomer thereof, for use in the manufacture of a medicament for treating an IKZF2-dependent and IKZF4-dependent disease or disorder.

In another aspect, the present disclosure relates to a method of reducing IKZF2 protein levels. The method comprises administering to the patient in need thereof a compound of Formula (I) or Embodiment 16, 17, or 35, or a pharmaceutically acceptable salt, hydrate, solvate, prodrug, stereoisomer, or tautomer thereof or a composition comprising a compound of Formula (I) or Embodiment 16, 17, or 35, or a pharmaceutically acceptable salt, hydrate, solvate, prodrug, stereoisomer, or tautomer thereof.

Another aspect of the present disclosure relates to a method of reducing IKZF2 and IKZF4 protein levels. The method comprises administering to the patient in need thereof a compound of Formula (I) or Embodiment 16, 17, or 35, or a pharmaceutically acceptable salt, hydrate, solvate, prodrug, stereoisomer, or tautomer thereof or a composition comprising a compound of Formula (I) or Embodiment 16, 17, or 35, or a pharmaceutically acceptable salt, hydrate, solvate, prodrug, stereoisomer, or tautomer thereof.

In another aspect, the present disclosure relates to a compound of Formula (I) or Embodiment 16, 17, or 35, or a pharmaceutically acceptable salt, hydrate, solvate, prodrug, stereoisomer, or tautomer thereof or a composition comprising a compound of Formula (I) or Embodiment 16, 17, or 35, or a pharmaceutically acceptable salt, hydrate, solvate, prodrug, stereoisomer, or tautomer thereof for use in the reduction of IKZF2 protein levels.

Another aspect of the present disclosure relates to a compound of Formula (I) or Embodiment 16, 17, or 35, or a pharmaceutically acceptable salt, hydrate, solvate, prodrug, stereoisomer, or tautomer thereof or a composition comprising a compound of Formula (I) or Embodiment 16, 17, or 35, or a pharmaceutically acceptable salt, hydrate, solvate, prodrug, stereoisomer, or tautomer thereof for use in the reduction of IKZF2 and IKZF4 protein levels.

In another aspect, the present disclosure relates to the use of a compound of Formula (I) or Embodiment 16, 17, or 35, or a pharmaceutically acceptable salt, hydrate, solvate, prodrug, stereoisomer, or tautomer thereof or a composition, in the manufacture of a medicament for reducing IKZF2 protein levels.

Another aspect of the present disclosure relates to the use of a compound of Formula (I) or Embodiment 16, 17, or 35, or a pharmaceutically acceptable salt, hydrate, solvate, prodrug, stereoisomer, or tautomer thereof or a composition comprising a compound of Formula (I) or Embodiment 16, 17, or 35, or a pharmaceutically acceptable salt, hydrate, solvate, prodrug, stereoisomer, or tautomer thereof, in the manufacture of a medicament for reducing IKZF2 and IKZF4 protein levels.

In another aspect, the present disclosure relates to a method of reducing IKZF2 protein levels, wherein reduction of IKZF2 protein levels treats or ameliorates the disease or disorder. The method comprises administering to the patient in need thereof a compound of Formula (I) or Embodiment 16, 17, or 35, or a pharmaceutically acceptable salt, hydrate, solvate, prodrug, stereoisomer, or tautomer thereof or a composition comprising a compound of Formula (I) or Embodiment 16, 17, or 35, or a pharmaceutically acceptable salt, hydrate, solvate, prodrug, stereoisomer, or tautomer thereof.

Another aspect of the present disclosure relates to a method of reducing IKZF2 and IKZF4 protein levels, wherein reduction of IKZF2 and IKZF4 protein levels treats or ameliorates the disease or disorder. The method comprises administering to the patient in need thereof a compound of Formula (I) or Embodiment 16, 17, or 35, or a pharmaceutically acceptable salt, hydrate, solvate, prodrug, stereoisomer, or tautomer thereof or a composition comprising a compound of Formula (I) or Embodiment 16, 17, or 35, or a pharmaceutically acceptable salt, hydrate, solvate, prodrug, stereoisomer, or tautomer thereof.

In another aspect, the present disclosure relates to a compound of Formula (I) or Embodiment 16, 17, or 35, or a pharmaceutically acceptable salt, hydrate, solvate, prodrug, stereoisomer, or tautomer thereof or a composition comprising a compound of Formula (I) or Embodiment 16, 17, or 35, or a pharmaceutically acceptable salt, hydrate, solvate, prodrug, stereoisomer, or tautomer thereof for use in the reduction of IKZF2 protein levels, wherein reduction of IKZF2 protein levels treats or ameliorates the disease or disorder.

Another aspect of the present disclosure relates to a compound of Formula (I) or Embodiment 16, 17, or 35, or a pharmaceutically acceptable salt, hydrate, solvate, prodrug, stereoisomer, or tautomer thereof or a composition comprising a compound of Formula (I) or Embodiment 16, 17, or 35, or a pharmaceutically acceptable salt, hydrate, solvate, prodrug, stereoisomer, or tautomer thereof for use in the reduction of IKZF2 and IKZF4 protein levels, wherein reduction of IKZF2 and IKZF4 protein levels treats or ameliorates the disease or disorder.

In another aspect, the present disclosure relates to the use of a compound of Formula (I) or Embodiment 16, 17, or 35, or a pharmaceutically acceptable salt, hydrate, solvate, prodrug, stereoisomer, or tautomer thereof or a composition, in the manufacture of a medicament for reducing IKZF2 protein levels, wherein reduction of IKZF2 protein levels treats or ameliorates the disease or disorder.

Another aspect of the present disclosure relates to the use of a compound of Formula (I) or Embodiment 16, 17, or 35, or a pharmaceutically acceptable salt, hydrate, solvate, prodrug, stereoisomer, or tautomer thereof or a composition comprising a compound of Formula (I) or Embodiment 16, 17, or 35, or a pharmaceutically acceptable salt, hydrate, solvate, prodrug, stereoisomer, or tautomer thereof, in the manufacture of a medicament for reducing IKZF2 and IKZF4 protein levels, wherein reduction of IKZF2 and IKZF4 protein levels treats or ameliorates the disease or disorder.

In another aspect, the present disclosure relates to a method of treating a disease or disorder by reducing IKZF2 protein levels, wherein reduction of IKZF2 protein levels treats or ameliorates the disease or disorder. The method comprises administering to the patient in need thereof a compound of Formula (I) or Embodiment 16, 17, or 35, or a pharmaceutically acceptable salt, hydrate, solvate, prodrug, stereoisomer, or tautomer thereof or a composition comprising a compound of Formula (I) or Embodiment 16, 17, or 35, or a pharmaceutically acceptable salt, hydrate, solvate, prodrug, stereoisomer, or tautomer thereof.

Another aspect of the present disclosure relates to a method of treating a disease or disorder by reducing IKZF2 and IKZF4 protein levels, wherein reduction of IKZF2 and IKZF4 protein levels treats or ameliorates the disease or disorder. The method comprises administering to the patient in need thereof a compound of Formula (I) or Embodiment 16, 17, or 35, or a pharmaceutically acceptable salt, hydrate, solvate, prodrug, stereoisomer, or tautomer thereof or a composition comprising a compound of Formula (I) or Embodiment 16, 17, or 35, or a pharmaceutically acceptable salt, hydrate, solvate, prodrug, stereoisomer, or tautomer thereof.

In another aspect, the present disclosure relates to a compound of Formula (I) or Embodiment 16, 17, or 35, or a pharmaceutically acceptable salt, hydrate, solvate, prodrug, stereoisomer, or tautomer thereof or a composition comprising a compound of Formula (I) or Embodiment 16, 17, or 35, or a pharmaceutically acceptable salt, hydrate, solvate, prodrug, stereoisomer, or tautomer thereof for use in the treatment of a disease or disorder by reducing IKZF2 protein levels, wherein reduction of IKZF2 protein levels treats or ameliorates the disease or disorder.

Another aspect of the present disclosure relates to a compound of Formula (I) or Embodiment 16, 17, or 35, or a pharmaceutically acceptable salt, hydrate, solvate, prodrug, stereoisomer, or tautomer thereof or a composition comprising a compound of Formula (I) or Embodiment 16, 17, or 35, or a pharmaceutically acceptable salt, hydrate, solvate, prodrug, stereoisomer, or tautomer thereof for use in the treatment of a disease or disorder by reducing IKZF2 and IKZF4 protein levels, wherein reduction of IKZF2 and IKZF4 protein levels treats or ameliorates the disease or disorder.

In another aspect, the present disclosure relates to the use of a compound of Formula (I) or Embodiment 16, 17, or 35, or a pharmaceutically acceptable salt, hydrate, solvate, prodrug, stereoisomer, or tautomer thereof or a composition, in the manufacture of a medicament for treating a disease or disorder by reducing IKZF2 protein levels, wherein reduction of IKZF2 protein levels treats or ameliorates the disease or disorder.

Another aspect of the present disclosure relates to the use of a compound of Formula (I) or Embodiment 16, 17, or 35, or a pharmaceutically acceptable salt, hydrate, solvate, prodrug, stereoisomer, or tautomer thereof or a composition comprising a compound of Formula (I) or Embodiment 16, 17, or 35, or a pharmaceutically acceptable salt, hydrate, solvate, prodrug, stereoisomer, or tautomer thereof, in the manufacture of a medicament for treating a disease or disorder by reducing IKZF2 and IKZF4 protein levels, wherein reduction of IKZF2 and IKZF4 protein levels treats or ameliorates the disease or disorder.

The compounds of the present disclosure can be used for the treatment, of a disease or disorder selected from liposarcoma, neuroblastoma, glioblastoma, bladder cancer, adrenocortical cancer, multiple myeloma, colorectal cancer, non-small cell lung cancer, Human Papilloma Virus-associated cervical, oropharyngeal, penis, anal, thyroid, or vaginal cancer or Epstein-Barr Virus-associated nasopharyngeal carcinoma, gastric cancer, rectal cancer, thyroid cancer, Hodgkin lymphoma or diffuse large B-cell lymphoma. the cancer is selected from prostate cancer, breast carcinoma, lymphomas, leukaemia, myeloma, bladder carcinoma, colon cancer, cutaneous melanoma, hepatocellular carcinoma, endometrial cancer, ovarian cancer, cervical cancer, lung cancer, renal cancer, glioblastoma multiform, glioma, thyroid cancer, parathyroid tumor, nasopharyngeal cancer, tongue cancer, pancreatic cancer, esophageal cancer, cholangiocarcinoma, gastric cancer, soft tissue sarcomas, rhabdomyosarcoma (RMS), synovial sarcoma, osteosarcoma, rhabdoid cancers, cancer for which the immune response is deficient, an immunogenic cancer, and Ewing's sarcoma. In one embodiment, the IKZF2-dependent disease or disorder is a disease or disorder is selected from non-small cell lung cancer (NSCLC), melanoma, triple-negative breast cancer (TNBC), nasopharyngeal cancer (NPC), microsatellite stable colorectal cancer (mssCRC), thymoma, carcinoid, and gastrointestinal stromal tumor (GIST). In another embodiment, the cancer is selected from non-small cell lung cancer (NSCLC), melanoma, triple-negative breast cancer (TNBC), nasopharyngeal cancer (NPC), microsatellite stable colorectal cancer (mssCRC), thymoma, carcinoid, acute myelogenous leukemia, and gastrointestinal stromal tumor (GIST). In another embodiment, the IKZF2-dependent disease or disorder is a disease or disorder is selected from non-small cell lung cancer (NSCLC), melanoma, triple-negative breast cancer (TNBC), nasopharyngeal cancer (NPC), and microsatellite stable colorectal cancer (mssCRC).

The disclosed compounds of the disclosure can be administered in effective amounts to treat or prevent a disorder and/or prevent the development thereof in subjects.

### G. Administration, Pharmaceutical Compositions, and Dosing of Compounds of the Disclosure

Administration of the disclosed compounds can be accomplished via any mode of administration for therapeutic agents. These modes include systemic or local administration such as oral, nasal, parenteral, transdermal, subcutaneous, vaginal, buccal, rectal or topical administration modes.

Depending on the intended mode of administration, the disclosed compositions can be in solid, semi-solid or liquid dosage form, such as, for example, injectables, tablets, suppositories, pills, time-release capsules, elixirs, tinctures, emulsions, syrups, powders, liquids, suspensions, or the like, sometimes in unit dosages and consistent with conventional pharmaceutical practices. Likewise, they can also be administered in intravenous (both bolus and infusion), intraperitoneal, subcutaneous or intramuscular form, and all using forms well known to those skilled in the pharmaceutical arts.

Illustrative pharmaceutical compositions are tablets and gelatin capsules comprising a compound of the disclosure and a pharmaceutically acceptable carrier, such as a) a diluent, e.g., purified water, triglyceride oils, such as hydrogenated or partially hydrogenated vegetable oil, or mixtures thereof, corn oil, olive oil, sunflower oil, safflower oil, fish oils, such as EPA or DHA, or their esters or triglycerides or mixtures thereof, omega-3 fatty acids or derivatives thereof, lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride, and/or polyethylene glycol; for tablets also; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes, and/or polyvinylpyrrolidone, if desired; d) a disintegrant, e.g., starches, agar, methyl cellulose, bentonite, xanthan gum, algic acid or its sodium salt, or effervescent mixtures; e) absorbent, colorant, flavorant and sweetener; f) an emulsifier or dispersing agent, such as Tween 80, Labrasol, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption of the compound such as cyclodextrin, hydroxypropyl-cyclodextrin, PEG400, PEG200.

Liquid, particularly injectable, compositions can, for example, be prepared by dissolution, dispersion, etc. For example, the disclosed compound is dissolved in or mixed with a pharmaceutically acceptable solvent such as, for example, water, saline, aqueous dextrose, glycerol, ethanol, and the like, to thereby form an injectable isotonic solution or suspension. Proteins such as albumin, chylomicron particles, or serum proteins can be used to solubilize the disclosed compounds.

The disclosed compounds can be also formulated as a suppository that can be prepared from fatty emulsions or suspensions; using polyalkylene glycols such as propylene glycol, as the carrier.

The disclosed compounds can also be administered in the form of liposome delivery systems, such as small unilamellar vesicles, large unilamellar vesicles, and multilamellar vesicles. Liposomes can be formed from a variety of phospholipids, containing cholesterol, stearylamine or phosphatidylcholines.

In some embodiments, a film of lipid components is hydrated with an aqueous solution of drug to a form lipid layer encapsulating the drug, as described in U.S. Pat. No. 5,262,564, which is hereby incorporated by reference in its entirety.

Disclosed compounds can also be delivered by the use of monoclonal antibodies as individual carriers to which the disclosed compounds are coupled. The disclosed compounds can also be coupled with soluble polymers as targetable drug carriers. Such polymers can include polyvinylpyrrolidone, pyran copolymer, polyhydroxypropylmethacrylamide-phenol, polyhydroxyethylaspanamidephenol, or polyethyleneoxidepolylysine substituted with palmitoyl residues. Furthermore, the disclosed compounds can be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates, and cross-linked or amphipathic block copolymers of hydrogels. In one embodiment, disclosed compounds are not covalently bound to a polymer, e.g., a polycarboxylic acid polymer, or a polyacrylate.

Parental injectable administration is generally used for subcutaneous, intramuscular or intravenous injections and infusions. Injectables can be prepared in conventional forms, either as liquid solutions or suspensions or solid forms suitable for dissolving in liquid prior to injection.

Another aspect of the disclosure is directed to pharmaceutical compositions comprising a compound of Formula (I), and a pharmaceutically acceptable carrier. The pharmaceutical acceptable carrier may further include an excipient, diluent, or surfactant.

Another aspect of the disclosure is directed to pharmaceutical compositions comprising a compound of any one of Embodiment 16, 17, or 35, and a pharmaceutically acceptable carrier. The pharmaceutical acceptable carrier may further include an excipient, diluent, or surfactant.

Compositions can be prepared according to conventional mixing, granulating or coating methods, respectively, and the present pharmaceutical compositions can contain from about 0.1% to about 99%, from about 5% to about 90%, or from about 1% to about 20% of the disclosed compound by weight or volume.

In one embodiment, the disclosure provides a kit comprising two or more separate pharmaceutical compositions, at least one of which contains a compound of the present disclosure. In one embodiment, the kit comprises means for separately retaining said compositions, such as a container, divided bottle, or divided foil packet. An example of such a kit is a blister pack, as typically used for the packaging of tablets, capsules and the like.

The kit of the disclosure may be used for administering different dosage forms, for example, oral and parenteral, for administering the separate compositions at different dosage intervals, or for titrating the separate compositions against one another. To assist compliance, the kit of the disclosure typically comprises directions for administration.

The dosage regimen utilizing the disclosed compound is selected in accordance with a variety of factors including type, species, age, weight, sex, and medical condition of the patient; the severity of the condition to be treated; the route of administration; the renal or hepatic function of the patient; and the particular disclosed compound employed. A physician or veterinarian of ordinary skill in the art can readily determine and prescribe the effective amount of the drug required to prevent, counter or arrest the progress of the condition.

Effective dosage amounts of the disclosed compounds, when used for the indicated effects, range from about 0.5 mg to about 5000 mg of the disclosed compound as needed to treat the condition. Compositions for in vivo or in vitro use can contain about 0.5, 5, 20, 50, 75, 100, 150, 250, 500, 750, 1000, 1250, 2500, 3500, or 5000 mg of the disclosed compound, or, in a range of from one amount to another amount in the list of doses. In one embodiment, the compositions are in the form of a tablet that can be scored.

### H. Combination Therapy

The compounds of the disclosure can be administered in therapeutically effective amounts in a combinational therapy with one or more therapeutic agents (pharmaceutical combinations) or modalities, e.g., non-drug therapies. For example, synergistic effects can occur with other cancer agents. Where the compounds of the application are administered in conjunction with other therapies, dosages of the co-administered compounds will of course vary depending on the type of co-drug employed, on the specific drug employed, on the condition being treated and so forth.

The compounds can be administered simultaneously (as a single preparation or separate preparation), sequentially, separately, or over a period of time to the other drug therapy or treatment modality. In general, a combination therapy envisions administration of two or more drugs during a single cycle or course of therapy. A therapeutic agent is, for example, a chemical compound, peptide, antibody, antibody fragment or nucleic acid, which is therapeutically active or enhances the therapeutic activity when administered to a patient in combination with a compound of the present disclosure.

In one aspect, a compound of Formula (I) or Embodiment 16, 17, or 35, or a pharmaceutically acceptable salt, hydrate, solvate, prodrug, stereoisomer, or tautomer thereof, of the present disclosure can be combined with other therapeutic agents, such as other anti-cancer agents, anti-allergic agents, anti-nausea agents (or anti-emetics), pain relievers, cytoprotective agents, and combinations thereof.

In some embodiments, the compounds of Formula (I), or Embodiment 16, 17, or 35, or a pharmaceutically acceptable salt, hydrate, solvate, prodrug, stereoisomer, or tautomer thereof of the present disclosure are administered in combination with one or more second agent(s) selected from a PD-1 inhibitor, a PD-L1 inhibitor, a LAG-3 inhibitor, a cytokine, an A2A antagonist, a GITR agonist, a TIM-3 inhibitor, a STING agonist, and a TLR7 agonist, to treat a disease, e.g., cancer.

In another embodiment, one or more chemotherapeutic agents are used in combination with the compounds of Formula (I), or Embodiment 16, 17, or 35, or a pharmaceutically acceptable salt, hydrate, solvate, prodrug, stereoisomer, or tautomer thereof, for treating a disease, e.g., cancer, wherein said chemotherapeutic agents include, but are not limited to, anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), vinorelbine (Navelbine®), epirubicin (Ellence®), oxaliplatin (Eloxatin®), exemestane (Aromasin®), letrozole (Femara®), and fulvestrant (Faslodex®).

In other embodiments, the compounds of Formula (I), or Embodiment 16, 17, or 35, or a pharmaceutically acceptable salt, hydrate, solvate, prodrug, stereoisomer, or tautomer thereof, of the present disclosure are used in combination with one or more other anti-HER2 antibodies, e.g., trastuzumab, pertuzumab, margetuximab, or HT-19 described above, or with other anti-HER2 conjugates, e.g., ado-trastuzumab emtansine (also known as Kadcyla®, or T-DM1).

In other embodiments, the compounds of Formula (I), or Embodiment 16, 17, or 35, or a pharmaceutically acceptable salt, hydrate, solvate, prodrug, stereoisomer, or tautomer thereof, of the present disclosure are used in combination with one or more tyrosine kinase inhibitors, including but not limited to, EGFR inhibitors, Her3 inhibitors, IGFR inhibitors, and Met inhibitors, for treating a disease, e.g., cancer.

For example, tyrosine kinase inhibitors include but are not limited to, Erlotinib hydrochloride (Tarceva®); Linifanib (N-[4-(3-amino-TH-indazol-4-yl)phenyl]-N′-(2-fluoro-5-methylphenyl)urea, also known as ABT 869, available from Genentech); Sunitinib malate (Sutent®); Bosutinib (4-[(2,4-dichloro-5-methoxyphenyl)amino]-6-methoxy-7-[3-(4-methylpiperazin-1-yl)propoxy]quinoline-3-carbonitrile, also known as SKI-606, and described in U.S. Pat. No. 6,780,996); Dasatinib (Sprycel®); Pazopanib (Votrient®); Sorafenib (Nexavar®); Zactima (ZD6474); and Imatinib or Imatinib mesylate (Gilvec® and Gleevec®).

Epidermal growth factor receptor (EGFR) inhibitors include but are not limited to, Erlotinib hydrochloride (Tarceva®), Gefitinib (Iressa®); N-[4-[(3-Chloro-4-fluorophenyl)amino]-7-[[(3″S″)-tetrahydro-3-furanyl]oxy]-6-quinazolinyl]-4(dimethylamino)-2-butenamide, Tovok®); Vandetanib (Caprelsa®); Lapatinib (Tykerb®); (3R,4R)-4-Amino-1-((4-((3-methoxyphenyl)amino)pyrrolo[2,1-f][1,2,4]triazin-5-yl)methyl)piperidin-3-ol (BMS690514); Canertinib dihydrochloride (CI-1033); 6-[4-[(4-Ethyl-1-piperazinyl)methyl]phenyl]-N-[(1R)-1-phenylethyl]-7H-Pyrrolo[2,3-d]pyrimidin-4-amine (AEE788, CAS 497839-62-0); Mubritinib (TAK165); Pelitinib (EKB569); Afatinib (Gilotrif@); Neratinib (HKI-272); N-[4-[[1-[(3-Fluorophenyl)methyl]-1H-indazol-5-yl]amino]-5-methylpyrrolo[2,1-f][1,2,4]triazin-6-yl]-carbamic acid, (3S)-3-morpholinylmethyl ester (BMS599626); N-(3,4-Dichloro-2-fluorophenyl)-6-methoxy-7-[[(3a,5,6a)-octahydro-2-methylcyclopenta[c]pyrrol-5-yl]methoxy]-4-quinazolinamine (XL647, CAS 781613-23-8); and 4-[4-[[(1R)-1-Phenylethyl]amino]-7H-pyrrolo[2,3-d]pyrimidin-6-yl]-phenol (PK166, CAS187724-61-4).

EGFR antibodies include but are not limited to, Cetuximab (Erbitux®); Panitumumab (Vectibix®); Matuzumab (EMD-72000); Nimotuzumab (hR3); Zalutumumab; TheraCIM h-R3; MDX0447 (CAS 339151-96-1); and ch806 (mAb-806, CAS 946414-09-1).

Other HER2 inhibitors include but are not limited to, Neratinib (HKI-272, (2E)-N-[4-[[3-chloro-4-[(pyridin-2-yl)methoxy]phenyl]amino]-3-cyano-7-ethoxyquinolin-6-yl]-4-(dimethylamino)but-2-enamide, and described PCT Publication No. WO 05/028443); Lapatinib or Lapatinib ditosylate (Tykerb®); (3R,4R)-4-amino-1-((4-((3-methoxyphenyl)amino)pyrrolo[2,1-f][1,2,4]triazin-5-yl)methyl)piperidin-3-ol (BMS690514); (2E)-N-[4-[(3-Chloro-4-fluorophenyl)amino]-7-[[(3S)-tetrahydro-3-furanyl]oxy]-6-quinazolinyl]-4-(dimethylamino)-2-butenamide (BIBW-2992, CAS 850140-72-6); N-[4-[[1-[(3-Fluorophenyl)methyl]-1H-indazol-5-yl]amino]-5-methylpyrrolo[2,1-f][1,2,4]triazin-6-yl]-carbamic acid, (3S)-3-morpholinylmethyl ester (BMS 599626, CAS 714971-09-2); Canertinib dihydrochloride (PD183805 or CI-1033); and N-(3,4-Dichloro-2-fluorophenyl)-6-methoxy-7-[[(3a,5,6a)-octahydro-2-methylcyclopenta[c]pyrrol-5-yl]methoxy]-4-quinazolinamine (XL647, CAS 781613-23-8).

HER3 inhibitors include but are not limited to, LJM716, MM-121, AMG-888, RG7116, REGN-1400, AV-203, MP-RM-1, MM-111, and MEHD-7945A.

MET inhibitors include but are not limited to, Cabozantinib (XL184, CAS 849217-68-1); Foretinib (GSK1363089, formerly XL880, CAS 849217-64-7); Tivantinib (ARQ197, CAS 1000873-98-2); 1-(2-Hydroxy-2-methylpropyl)-N-(5-(7-methoxyquinolin-4-yloxy)pyridin-2-yl)-5-methyl-3-oxo-2-phenyl-2,3-dihydro-TH-pyrazole-4-carboxamide (AMG 458); Cryzotinib (Xalkori®, PF-02341066); (3Z)-5-(2,3-Dihydro-1H-indol-1-ylsulfonyl)-3-({3,5-dimethyl-4-[(4-methylpiperazin-1-yl)carbonyl]-1H-pyrrol-2-yl}methylene)-1,3-dihydro-2H-indol-2-one (SU11271); (3Z)—N-(3-Chlorophenyl)-3-({3,5-dimethyl-4-[(4-methylpiperazin-1-yl)carbonyl]-1H-pyrrol-2-yl}methylene)-N-methyl-2-oxoindoline-5-sulfonamide (SU11274); (3Z)—N-(3-Chlorophenyl)-3-{[3,5-dimethyl-4-(3-morpholin-4-ylpropyl)-1H-pyrrol-2-yl]methylene}-N-methyl-2-oxoindoline-5-sulfonamide (SU11606); 6-[Difluoro[6-(1-methyl-1Hpyrazol-4-yl)-1,2,4-triazolo[4,3-b]pyridazin-3-yl]methyl]-quinoline (JNJ38877605, CAS 943540-75-8); 2-[4-[1-(Quinolin-6-ylmethyl)-1H-[1,2,3]triazolo[4,5-b]pyrazin-6-yl]-1H-pyrazol-1-yl]ethanol (PF04217903, CAS 956905-27-4); N-((2R)-1,4-Dioxan-2-ylmethyl)-N-methyl-N′-[3-(1-methyl-1H-pyrazol-4-yl)-5-oxo-5H-benzo[4,5]cyclohepta[1,2-b]pyridin-7-yl]sulfamide (MK2461, CAS 917879-39-1); 6-[[6-(1-Methyl-1H-pyrazol-4-yl)-1,2,4-triazolo[4,3-b]pyridazin 3-yl]thio]-quinoline (SGX523, CAS 1022150-57-7); and (3Z)-5-[[(2,6-Dichlorophenyl)methyl]sulfonyl]-3-[[3,5-dimethyl-4-[[(2R)-2-(1-pyrrolidinylmethyl)-1-pyrrolidinyl]carbonyl]-1H-pyrrol-2-yl]methylene]-1,3-dihydro-2H-indol-2-one (PHA665752, CAS 477575-56-7).

IGFR inhibitors include but are not limited to, BMS-754807, XL-228, OSI-906, GSK0904529A, A-928605, AXL1717, KW-2450, MK0646, AMG479, IMCA12, MEDI-573, and B1836845. See e.g., Yee, JNCI, 104; 975 (2012) for review.

In another embodiment, the compounds of Formula (I), or Embodiment 16, 17, or 35 of the present disclosure are used in combination with one or more proliferation signaling pathway inhibitors, including but not limited to, MEK inhibitors, BRAF inhibitors, PI3K/Akt inhibitors, SHP2 inhibitors, and also mTOR inhibitors, and CDK inhibitors, for treating a disease, e.g., cancer.

For example, mitogen-activated protein kinase (MEK) inhibitors include but are not limited to, XL-518 (also known as GDC-0973, Cas No. 1029872-29-4, available from ACC Corp.); 2-[(2-Chloro-4-iodophenyl)amino]-N-(cyclopropylmethoxy)-3,4-difluoro-benzamide (also known as CI-1040 or PD184352 and described in PCT Publication No. WO2000035436); N-[(2R)-2,3-Dihydroxypropoxy]-3,4-difluoro-2-[(2-fluoro-4-iodophenyl)amino]-benzamide (also known as PD0325901 and described in PCT Publication No. WO2002006213); 2,3-Bis[aminol[(2-aminophenyl)thio]methylene]-butanedinitrile (also known as U0126 and described in U.S. Pat. No. 2,779,780); N-[3,4-Difluoro-2-[(2-fluoro-4-iodophenyl)amino]-6-methoxyphenyl]-1-[(2R)-2,3-dihydroxypropyl]-cyclopropanesulfonamide (also known as RDEA119 or BAY869766 and described in PCT Publication No. WO2007014011); (3S,4R,5Z,8S,9S,11E)-14-(Ethylamino)-8,9,16-trihydroxy-3,4-dimethyl-3,4,9, 19-tetrahydro-1H-2-benzoxacyclotetradecine-1,7(8H)-dione] (also known as E6201 and described in PCT Publication No. WO2003076424); 2′-Amino-3′-methoxyflavone (also known as PD98059 available from Biaffin GmbH & Co., KG, Germany); Vemurafenib (PLX-4032, CAS 918504-65-1); (R)-3-(2,3-Dihydroxypropyl)-6-fluoro-5-(2-fluoro-4-iodophenylamino)-8-methylpyrido[2,3-d]pyrimidine-4,7(3H,8H)-dione (TAK-733, CAS 1035555-63-5); Pimasertib (AS-703026, CAS 1204531-26-9); and Trametinib dimethyl sulfoxide (GSK-1120212, CAS 1204531-25-80).

BRAF inhibitors include, but are not limited to, Vemurafenib (or Zelboraf®), GDC-0879, PLX-4720 (available from Symansis), Dabrafenib (or GSK2118436), LGX 818, CEP-32496, UI-152, RAF 265, Regorafenib (BAY 73-4506), CCT239065, or Sorafenib (or Sorafenib Tosylate, or Nexavar®), or Ipilimumab (or MDX-010, MDX-101, or Yervoy).

Phosphoinositide 3-kinase (PI3K) inhibitors include, but are not limited to, 4-[2-(1H-Indazol-4-yl)-6-[[4-(methylsulfonyl)piperazin-1-yl]methyl]thieno[3,2-d]pyrimidin-4-yl]morpholine (also known as GDC0941, RG7321, GNE0941, Pictrelisib, or Pictilisib; and described in PCT Publication Nos. WO 09/036082 and WO 09/055730); Tozasertib (VX680 or MK-0457, CAS 639089-54-6); (5Z)-5-[[4-(4-Pyridinyl)-6-quinolinyl]methylene]-2,4-thiazolidinedione (GSK1059615, CAS 958852-01-2); (1E,4S,4aR,5R,6aS,9aR)-5-(Acetyloxy)-1-[(di-2-propenylamino)methylene]-4,4a,5,6,6a,8,9,9a-octahydro-11-hydroxy-4-(methoxymethyl)-4a,6a-dimethylcyclopenta[5,6]naphtho[1,2-c]pyran-2,7,10(1H)-trione (PX866, CAS 502632-66-8); 8-Phenyl-2-(morpholin-4-yl)-chromen-4-one (LY294002, CAS 154447-36-6); (S)-N1-(4-methyl-5-(2-(1,1,1-trifluoro-2-methylpropan-2-yl)pyridin-4-yl)thiazol-2-yl)pyrrolidine-1,2-dicarboxamide (also known as BYL719 or Alpelisib); 2-(4-(2-(1-isopropyl-3-methyl-1H-1,2,4-triazol-5-yl)-5,6-dihydrobenzo[f]imidazo[1,2-d][1,4]oxazepin-9-yl)-1H-pyrazol-1-yl)-2-methylpropanamide (also known as GDC0032, RG7604, or Taselisib). mTOR inhibitors include but are not limited to, Temsirolimus (Torisel®); Ridaforolimus (formally known as deferolimus, (1R,2R,4S)-4-[(2R)-2 [(1R,9S,12S,15R,16E,18R,19R,21R,23S,24E,26E,28Z,30S,32S,35R)-1,18-dihydroxy-19,30-dimethoxy-15,17,21,23, 29,35-hexamethyl-2,3,10,14,20-pentaoxo-11,36-dioxa-4-azatricyclo[30.3.1.04,9]hexatriaconta-16,24,26,28-tetraen-12-yl]propyl]-2-methoxycyclohexyl dimethylphosphinate, also known as AP23573 and MK8669, and described in PCT Publication No. WO 03/064383); Everolimus (Afinitor® or RAD001); Rapamycin (AY22989, Sirolimus®); Simapimod (CAS 164301-51-3); (5-{2,4-Bis[(3S)-3-methylmorpholin-4-yl]pyrido[2,3-d]pyrimidin-7-yl}-2-methoxyphenyl)methanol (AZD8055); 2-Amino-8-[trans-4-(2-hydroxyethoxy)cyclohexyl]-6-(6-methoxy-3-pyridinyl)-4-methyl-pyrido[2,3-d]pyrimidin-7(8H)-one (PF04691502, CAS 1013101-36-4); and N²-[1,4-dioxo-4-[[4-(4-oxo-8-phenyl-4H-1-benzopyran-2-yl)morpholinium-4-yl]methoxy]butyl]-L-arginylglycyl-L-aspartylL-serine-, inner salt (SF1126, CAS 936487-67-1).

CDK inhibitors include but are not limited to, Palbociclib (also known as PD-0332991, Ibrance®, 6-Acetyl-8-cyclopentyl-5-methyl-2-{[5-(1-piperazinyl)-2-pyridinyl]amino}pyrido[2,3-d]pyrimidin-7(8H)-one).

In yet another embodiment, the compounds of Formula (I), or Embodiment 16, 17, or 35, or a pharmaceutically acceptable salt, hydrate, solvate, prodrug, stereoisomer, or tautomer thereof, of the present disclosure are used in combination with one or more pro-apoptotics, including but not limited to, IAP inhibitors, BCL2 inhibitors, MCL1 inhibitors, TRAIL agents, CHK inhibitors, for treating a disease, e.g., cancer.

For examples, IAP inhibitors include but are not limited to, LCL161, GDC-0917, AEG-35156, AT406, and TL32711. Other examples of IAP inhibitors include but are not limited to those disclosed in WO04/005284, WO 04/007529, WO05/097791, WO 05/069894, WO 05/069888, WO 05/094818, US2006/0014700, US2006/0025347, WO 06/069063, WO 06/010118, WO 06/017295, and WO08/134679, all of which are incorporated herein by reference.

BCL-2 inhibitors include but are not limited to, 4-[4-[[2-(4-Chlorophenyl)-5,5-dimethyl-1-cyclohexen-1-yl]methyl]-1-piperazinyl]-N-[[4-[[(1R)-3-(4-morpholinyl)-1-[(phenylthio)methyl]propyl]amino]-3-[(trifluoromethyl)sulfonyl]phenyl]sulfonyl]benzamide (also known as ABT-263 and described in PCT Publication No. WO 09/155386); Tetrocarcin A; Antimycin; Gossypol ((−)BL-193); Obatoclax; Ethyl-2-amino-6-cyclopentyl-4-(1-cyano-2-ethoxy-2-oxoethyl)-4Hchromone-3-carboxylate (HA14-1); Oblimersen (G3139, Genasense®); Bak BH3 peptide; (−)-Gossypol acetic acid (AT-101); 4-[4-[(4′-Chloro[1,1′-biphenyl]-2-yl)methyl]-1-piperazinyl]-N-[[4-[[(1R)-3-(dimethylamino)-1-[(phenylthio)methyl]propyl]amino]-3-nitrophenyl]sulfonyl]-benzamide (ABT-737, CAS 852808-04-9); and Navitoclax (ABT-263, CAS 923564-51-6).

Proapoptotic receptor agonists (PARAs) including DR4 (TRAILR1) and DR5 (TRAILR2), including but are not limited to, Dulanermin (AMG-951, RhApo2L/TRAIL); Mapatumumab (HRS-ETR1, CAS 658052-09-6); Lexatumumab (HGS-ETR2, CAS 845816-02-6); Apomab (Apomab®); Conatumumab (AMG655, CAS 896731-82-1); and Tigatuzumab(CS1008, CAS 946415-34-5, available from Daiichi Sankyo).

Checkpoint Kinase (CHK) inhibitors include but are not limited to, 7-Hydroxystaurosporine (UCN-01); 6-Bromo-3-(1-methyl-1H-pyrazol-4-yl)-5-(3R)-3-piperidinylpyrazolo[1,5-a]pyrimidin-7-amine (SCH900776, CAS 891494-63-6); 5-(3-Fluorophenyl)-3-ureidothiophene-2-carboxylic acid N—[(S)-piperidin-3-yl]amide (AZD7762, CAS 860352-01-8); 4-[((3S)-1-Azabicyclo[2.2.2]oct-3-yl)amino]-3-(1H-benzimidazol-2-yl)-6-chloroquinolin-2(1H)-one (CHIR 124, CAS 405168-58-3); 7-Aminodactinomycin (7-AAD), Isogranulatimide, debromohymenialdisine; N-[5-Bromo-4-methyl-2-[(2S)-2-morpholinylmethoxy]-phenyl]-N′-(5-methyl-2-pyrazinyl)urea (LY2603618, CAS 911222-45-2); Sulforaphane (CAS 4478-93-7, 4-Methylsulfinylbutyl isothiocyanate); 9,10,11,12-Tetrahydro-9,12-epoxy-1H-diindolo[1,2,3-fg:3′,2′,1′-kl]pyrrolo[3,4-i][1,6]benzodiazocine-1,3(2H)-dione (SB-218078, CAS 135897-06-2); and TAT-S216A (YGRKKRRQRRRLYRSPAMPENL (SEQ ID NO: 33)), and CBP501 ((d-Bpa)sws(d-Phe-F5)(d-Cha)rrrqrr).

In a further embodiment, the compounds of Formula (I), or Embodiment 16, 17, or 35, or a pharmaceutically acceptable salt, hydrate, solvate, prodrug, stereoisomer, or tautomer thereof, of the present disclosure are used in combination with one or more immunomodulators (e.g., one or more of an activator of a costimulatory molecule or an inhibitor of an immune checkpoint molecule), for treating a disease, e.g., cancer.

In certain embodiments, the immunomodulator is an activator of a costimulatory molecule. In one embodiment, the agonist of the costimulatory molecule is selected from an agonist (e.g., an agonistic antibody or antigen-binding fragment thereof, or a soluble fusion) of OX40, CD2, CD27, CDS, ICAM-1, LFA-1 (CD11a/CD18), ICOS (CD278), 4-1BB (CD137), GITR, CD30, CD40, BAFFR, HVEM, CD7, LIGHT, NKG2C, SLAMF7, NKp80, CD160, B7-H3 or CD83 ligand.

### GITR Agonists

In some embodiments, a GITR agonist is used in combination with a compound of Formula (I) or Embodiment 16, 17, or 35, or a pharmaceutically acceptable salt, hydrate, solvate, prodrug, stereoisomer, or tautomer thereof, for treating a disease, e.g., cancer. In some embodiments, the GITR agonist is GWN323 (Novartis), BMS-986156, MK-4166 or MK-1248 (Merck), TRX518 (Leap Therapeutics), INCAGN1876 (Incyte/Agenus), AMG 228 (Amgen) or INBRX-110 (Inhibrx).

### Exemplary GITR Agonists

In one embodiment, the GITR agonist is an anti-GITR antibody molecule. In one embodiment, the GITR agonist is an anti-GITR antibody molecule as described in WO 2016/057846, published on Apr. 14, 2016, entitled “Compositions and Methods of Use for Augmented Immune Response and Cancer Therapy,” incorporated by reference in its entirety.

In one embodiment, the anti-GITR antibody molecule comprises at least one, two, three, four, five or six complementarity determining regions (CDRs) (or collectively all of the CDRs) from a heavy and light chain variable region comprising an amino acid sequence shown in Table 1 (e.g., from the heavy and light chain variable region sequences of MAB7 disclosed in Table 1), or encoded by a nucleotide sequence shown in Table 1. In some embodiments, the CDRs are according to the Kabat definition (e.g., as set out in Table 1). In some embodiments, the CDRs are according to the Chothia definition (e.g., as set out in Table 1). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 1, or encoded by a nucleotide sequence shown in Table 1.

In one embodiment, the anti-GITR antibody molecule comprises a heavy chain variable region (VH) comprising a VHCDR1 amino acid sequence of SEQ ID NO: 9, a VHCDR2 amino acid sequence of SEQ ID NO: 11, and a VHCDR3 amino acid sequence of SEQ ID NO: 13; and a light chain variable region (VL) comprising a VLCDR1 amino acid sequence of SEQ ID NO: 14, a VLCDR2 amino acid sequence of SEQ ID NO: 16, and a VLCDR3 amino acid sequence of SEQ ID NO: 18, each disclosed in Table 1.

In one embodiment, the anti-GITR antibody molecule comprises a VH comprising the amino acid sequence of SEQ ID NO: 1, or an amino acid sequence at least 85%, 90%, 95%, or 99% identical or higher to SEQ ID NO: 1. In one embodiment, the anti-GITR antibody molecule comprises a VL comprising the amino acid sequence of SEQ ID NO: 2, or an amino acid sequence at least 85%, 90%, 95%, or 99% identical or higher to SEQ ID NO: 2. In one embodiment, the anti-GITR antibody molecule comprises a VH comprising the amino acid sequence of SEQ ID NO: 1 and a VL comprising the amino acid sequence of SEQ ID NO: 2.

In one embodiment, the antibody molecule comprises a VH encoded by the nucleotide sequence of SEQ ID NO: 5, or a nucleotide sequence at least 85%, 90%, 95%, or 99% identical or higher to SEQ ID NO: 5. In one embodiment, the antibody molecule comprises a VL encoded by the nucleotide sequence of SEQ ID NO: 6, or a nucleotide sequence at least 85%, 90%, 95%, or 99% identical or higher to SEQ ID NO: 6. In one embodiment, the antibody molecule comprises a VH encoded by the nucleotide sequence of SEQ ID NO: 5 and a VL encoded by the nucleotide sequence of SEQ ID NO: 6.

In one embodiment, the anti-GITR antibody molecule comprises a heavy chain comprising the amino acid sequence of SEQ ID NO: 3, or an amino acid sequence at least 85%, 90%, 95%, or 99% identical or higher to SEQ ID NO: 3. In one embodiment, the anti-GITR antibody molecule comprises a light chain comprising the amino acid sequence of SEQ ID NO: 4, or an amino acid sequence at least 85%, 90%, 95%, or 99% identical or higher to SEQ ID NO: 4. In one embodiment, the anti-GITR antibody molecule comprises a heavy chain comprising the amino acid sequence of SEQ ID NO: 3 and a light chain comprising the amino acid sequence of SEQ ID NO: 4.

In one embodiment, the antibody molecule comprises a heavy chain encoded by the nucleotide sequence of SEQ ID NO: 7, or a nucleotide sequence at least 85%, 90%, 95%, or 99% identical or higher to SEQ ID NO: 7. In one embodiment, the antibody molecule comprises a light chain encoded by the nucleotide sequence of SEQ ID NO: 8, or a nucleotide sequence at least 85%, 90%, 95%, or 99% identical or higher to SEQ ID NO: 8. In one embodiment, the antibody molecule comprises a heavy chain encoded by the nucleotide sequence of SEQ ID NO: 7 and a light chain encoded by the nucleotide sequence of SEQ ID NO: 8.

The antibody molecules described herein can be made by vectors, host cells, and methods described in WO 2016/057846, incorporated by reference in its entirety.

TABLE 1
Amino acid and nucleotide sequences of exemplary anti-GITR
antibody molecule

| MAB7 | | |
|---|---|---|
| SEQ ID NO: | VH | EVQLVESGGGLVQSGGSLRLSCAASGFSLSSYGVDWVRQAPGKGLEW |
| 1 | | VGVIWGGGGTYYASSLMGRFTISRDNSKNTLYLQMNSLRAEDTAVYY |
| | | CARHAYGHDGGFAMDYWGQGTLVTVSS |
| SEQ ID NO: | VL | EIVMTQSPATLSVSPGERATLSCRASESVSSNVAWYQQRPGQAPRLLIY |
| 2 | | GASNRATGIPARFSGSGSGTDFTLTISRLEPEDFAVYYCGQSYSYPFTFG |
| | | QGTKLEIK |
| SEQ ID NO: | Heavy | EVQLVESGGGLVQSGGSLRLSCAASGFSLSSYGVDWVRQAPGKGLEW |
| 3 | Chain | VGVIWGGGGTYYASSLMGRFTISRDNSKNTLYLQMNSLRAEDTAVYY |
| | | CARHAYGHDGGFAMDYWGQGTLVTVSSASTKGPSVFPLAPSSKSTSG |
| | | GTAALGCLVKDYFPEPVTVSWNSGALTSGVHTFPAVLQSSGLYSLSSV |
| | | VTVPSSSLGTQTYICNVNHKPSNTKVDKRVEPKSCDKTHTCPPCPAPEL |
| | | LGGPSVFLFPPKPKDTLMISRTPEVTCVVVDVSHEDPEVKFNWYVDGV |
| | | EVHNAKTKPREEQYNSTYRVVSVLTVLHQDWLNGKEYKCKVSNKAL |
| | | PAPIEKTISKAKGQPREPQVYTLPPSREEMTKNQVSLTCLVKGFYPSDI |
| | | AVEWESNGQPENNYKTTPPVLDSDGSFFLYSKLTVDKSRWQQGNVFS |
| | | CSVMHEALHNHYTQKSLSLSPGK |
| SEQ ID NO: | Light | EIVMTQSPATLSVSPGERATLSCRASESVSSNVAWYQQRPGQAPRLLIY |
| 4 | Chain | GASNRATGIPARFSGSGSGTDFTLTISRLEPEDFAVYYCGQSYSYPFTFG |
| | | QGTKLEIKRTVAAPSVFIFPPSDEQLKSGTASVVCLLNNFYPREAKVQ |
| | | WKVDNALQSGNSQESVTEQDSKDSTYSLSSTLTLSKADYEKHKVYAC |
| | | EVTHQGLSSPVTKSFNRGEC |
| SEQ ID NO: | DNA | GAGGTGCAGCTGGTGGAATCTGGCGGCGGACTGGTGCAGTCCGGC |
| 5 | VH | GGCTCTCTGAGACTGTCTTGCGCTGCCTCCGGCTTCTCCCTGTCCTC |
| | | TTACGGCGTGGACTGGGTGCGACAGGCCCCTGGCAAGGGCCTGGA |
| | | ATGGGTGGGAGTGATCTGGGGCGGAGGCGGCACCTACTACGCCTCT |
| | | TCCCTGATGGGCCGGTTCACCATCTCCCGGGACAACTCCAAGAACA |
| | | CCCTGTACCTGCAGATGAACTCCCTGCGGGCCGAGGACACCGCCGT |
| | | GTACTACTGCGCCAGACACGCCTACGGCCACGACGGCGGCTTCGCC |
| | | ATGGATTATTGGGGCCAGGGCACCCTGGTGACAGTGTCCTCC |
| SEQ ID NO: | DNA | GAGATCGTGATGACCCAGTCCCCCGCCACCCTGTCTGTGTCTCCCG |
| 6 | VL | GCGAGAGAGCCACCCTGAGCTGCAGAGCCTCCGAGTCCGTGTCCTC |
| | | CAACGTGGCCTGGTATCAGCAGAGACCTGGTCAGGCCCCTCGGCTG |
| | | CTGATCTACGGCGCCTCTAACCGGGCCACCGGCATCCCTGCCAGAT |
| | | TCTCCGGCTCCGGCAGCGGCACCGACTTCACCCTGACCATCTCCCG |
| | | GCTGGAACCCGAGGACTTCGCCGTGTACTACTGCGGCCAGTCCTAC |
| | | TCATACCCCTTCACCTTCGGCCAGGGCACCAAGCTGGAAATCAAG |
| SEQ ID NO: | DNA | GAGGTGCAGCTGGTGGAATCTGGCGGCGGACTGGTGCAGTCCGGC |
| 7 | Heavy | GGCTCTCTGAGACTGTCTTGCGCTGCCTCCGGCTTCTCCCTGTCCTC |
| | Chain | TTACGGCGTGGACTGGGTGCGACAGGCCCCTGGCAAGGGCCTGGA |
| | | ATGGGTGGGAGTGATCTGGGGCGGAGGCGGCACCTACTACGCCTCT |
| | | TCCCTGATGGGCCGGTTCACCATCTCCCGGGACAACTCCAAGAACA |
| | | CCCTGTACCTGCAGATGAACTCCCTGCGGGCCGAGGACACCGCCGT |
| | | GTACTACTGCGCCAGACACGCCTACGGCCACGACGGCGGCTTCGCC |
| | | ATGGATTATTGGGGCCAGGGCACCCTGGTGACAGTGTCCTCCGCTA |
| | | GCACCAAGGGCCCAAGTGTGTTTCCCCTGGCCCCCAGCAGCAAGTC |
| | | TACTTCCGGCGGAACTGCTGCCCTGGGTTGCCTGGTGAAGGACTAC |
| | | TTCCCCGAGCCCGTGACAGTGTCCTGGAACTCTGGGGCTCTGACTT |
| | | CCGGCGTGCACACCTTCCCCGCCGTGCTGCAGAGCAGCGGCCTGTA |
| | | CAGCCTGAGCAGCGTGGTGACAGTGCCCTCCAGCTCTCTGGGAACC |
| | | CAGACCTATATCTGCAACGTGAACCACAAGCCCAGCAACACCAAG |
| | | GTGGACAAGAGAGTGGAGCCCAAGAGCTGCGACAAGACCCACACC |
| | | TGCCCCCCCTGCCCAGCTCCAGAACTGCTGGGAGGGCCTTCCGTGT |
| | | TCCTGTTCCCCCCCAAGCCCAAGGACACCCTGATGATCAGCAGGAC |
| | | CCCCGAGGTGACCTGCGTGGTGGTGGACGTGTCCCACGAGGACCCA |
| | | GAGGTGAAGTTCAACTGGTACGTGGACGGCGTGGAGGTGCACAAC |
| | | GCCAAGACCAAGCCCAGAGAGGAGCAGTACAACAGCACCTACAGG |
| | | GTGGTGTCCGTGCTGACCGTGCTGCACCAGGACTGGCTGAACGGCA |
| | | AAGAATACAAGTGCAAAGTCTCCAACAAGGCCCTGCCAGCCCCAA |
| | | TCGAAAAGACAATCAGCAAGGCCAAGGGCCAGCCACGGGAGCCCC |
| | | AGGTGTACACCCTGCCCCCCAGCCGGGAGGAGATGACCAAGAACC |
| | | AGGTGTCCCTGACCTGTCTGGTGAAGGGCTTCTACCCCAGCGATAT |
| | | CGCCGTGGAGTGGGAGAGCAACGGCCAGCCCGAGAACAACTACAA |
| | | GACCACCCCCCCAGTGCTGGACAGCGACGGCAGCTTCTTCCTGTAC |
| | | AGCAAGCTGACCGTGGACAAGTCCAGGTGGCAGCAGGGCAACGTG |
| | | TTCAGCTGCAGCGTGATGCACGAGGCCCTGCACAACCACTACACCC |
| | | AGAAGTCCCTGAGCCTGAGCCCCGGCAAG |
| SEQ ID NO: | DNA | GAGATCGTGATGACCCAGTCCCCCGCCACCCTGTCTGTGTCTCCCG |
| 8 | Light | GCGAGAGAGCCACCCTGAGCTGCAGAGCCTCCGAGTCCGTGTCCTC |
| | Chain | CAACGTGGCCTGGTATCAGCAGAGACCTGGTCAGGCCCCTCGGCTG |
| | | CTGATCTACGGCGCCTCTAACCGGGCCACCGGCATCCCTGCCAGAT |
| | | TCTCCGGCTCCGGCAGCGGCACCGACTTCACCCTGACCATCTCCCG |
| | | GCTGGAACCCGAGGACTTCGCCGTGTACTACTGCGGCCAGTCCTAC |
| | | TCATACCCCTTCACCTTCGGCCAGGGCACCAAGCTGGAAATCAAGC |
| | | GTACGGTGGCCGCTCCCAGCGTGTTCATCTTCCCCCCCAGCGACGA |
| | | GCAGCTGAAGAGCGGCACCGCCAGCGTGGTGTGCCTGCTGAACAA |
| | | CTTCTACCCCCGGGAGGCCAAGGTGCAGTGGAAGGTGGACAACGC |
| | | CCTGCAGAGCGGCAACAGCCAGGAGAGCGTCACCGAGCAGGACAG |
| | | CAAGGACTCCACCTACAGCCTGAGCAGCACCCTGACCCTGAGCAAG |
| | | GCCGACTACGAGAAGCATAAGGTGTACGCCTGCGAGGTGACCCAC |
| | | CAGGGCCTGTCCAGCCCCGTGACCAAGAGCTTCAACAGGGGCGAG |
| | | TGC |
| SEQ ID NO: | HCDR1 | SYGVD |
| 9 (KABAT) | | |
| SEQ ID NO: | HCDR1 | GFSLSSY |
| 10 | | |
| (CHOTHIA) | | |
| SEQ ID NO: | HCDR2 | VIWGGGGTYYASSLMG |
| 11 (KABAT) | | |
| SEQ ID NO: | HCDR2 | WGGGG |
| 12 | | |
| (CHOTHIA) | | |
| SEQ ID NO: | HCDR3 | HAYGHDGGFAMDY |
| 13 (KABAT) | | |
| SEQ ID NO: | HCDR3 | HAYGHDGGFAMDY |
| 13 | | |
| (CHOTHIA) | | |
| SEQ ID NO: | LCDR1 | RASESVSSNVA |
| 14 (KABAT) | | |
| SEQ ID NO: | LCDR1 | SESVSSN |
| 15 | | |
| (CHOTHIA) | | |
| SEQ ID NO: | LCDR2 | GASNRAT |
| 16 (KABAT) | | |
| SEQ ID NO: | LCDR2 | GAS |
| 17 | | |
| (CHOTHIA) | | |
| SEQ ID NO: | LCDR3 | GQSYSYPFT |
| 18 (KABAT) | | |
| SEQ ID NO: | LCDR3 | SYSYPF |
| 19 | | |
| (CHOTHIA) | | |

### Other Exemplary GITR Agonists

In one embodiment, the anti-GITR antibody molecule is BMS-986156 (Bristol-Myers Squibb), also known as BMS 986156 or BMS986156. BMS-986156 and other anti-GITR antibodies are disclosed, e.g., in U.S. Pat. No. 9,228,016 and WO 2016/196792, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of BMS-986156, e.g., as disclosed in Table 2.

In one embodiment, the anti-GITR antibody molecule is MK-4166 or MK-1248 (Merck). MK-4166, MK-1248, and other anti-GITR antibodies are disclosed, e.g., in U.S. Pat. No. 8,709,424, WO 2011/028683, WO 2015/026684, and Mahne et al. Cancer Res. 2017; 77(5):1108-1118, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of MK-4166 or MK-1248.

In one embodiment, the anti-GITR antibody molecule is TRX518 (Leap Therapeutics). TRX518 and other anti-GITR antibodies are disclosed, e.g., in U.S. Pat. Nos. 7,812,135, 8,388,967, 9,028,823, WO 2006/105021, and Ponte J et al. (2010) Clinical Immunology; 135:S96, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of TRX518.

In one embodiment, the anti-GITR antibody molecule is INCAGN1876 (Incyte/Agenus). INCAGN1876 and other anti-GITR antibodies are disclosed, e.g., in US 2015/0368349 and WO 2015/184099, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of INCAGN1876.

In one embodiment, the anti-GITR antibody molecule is AMG 228 (Amgen). AMG 228 and other anti-GITR antibodies are disclosed, e.g., in U.S. Pat. No. 9,464,139 and WO 2015/031667, incorporated by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of AMG 228.

In one embodiment, the anti-GITR antibody molecule is INBRX-110 (Inhibrx). INBRX-110 and other anti-GITR antibodies are disclosed, e.g., in US 2017/0022284 and WO 2017/015623, incorporated by reference in their entirety. In one embodiment, the GITR agonist comprises one or more of the CDR sequences (or collectively all of the CDR sequences), the heavy chain or light chain variable region sequence, or the heavy chain or light chain sequence of INBRX-110.

In one embodiment, the GITR agonist (e.g., a fusion protein) is MEDI 1873 (MedImmune), also known as MED11873. MEDI 1873 and other GITR agonists are disclosed, e.g., in US 2017/0073386, WO 2017/025610, and Ross et al. Cancer Res 2016; 76(14 Suppl): Abstract nr 561, incorporated by reference in their entirety. In one embodiment, the GITR agonist comprises one or more of an IgG Fc domain, a functional multimerization domain, and a receptor binding domain of a glucocorticoid-induced TNF receptor ligand (GITRL) of MEDI 1873.

Further known GITR agonists (e.g., anti-GITR antibodies) include those described, e.g., in WO 2016/054638, incorporated by reference in its entirety.

In one embodiment, the anti-GITR antibody is an antibody that competes for binding with, and/or binds to the same epitope on GITR as, one of the anti-GITR antibodies described herein.

In one embodiment, the GITR agonist is a peptide that activates the GITR signaling pathway. In one embodiment, the GITR agonist is an immunoadhesin binding fragment (e.g., an immunoadhesin binding fragment comprising an extracellular or GITR binding portion of GITRL) fused to a constant region (e.g., an Fc region of an immunoglobulin sequence).

TABLE 2
Amino acid sequence of other exemplary
anti-GITR antibody molecules

| | | |
|---|---|---|
| BMS-986156 | | |
| SEQ ID NO: | VH | QVQLVESGGGVVQPGRSLRLSCAASGFTFSSYGMHWV |
| 20 | | RQAPGKGLEWVAVIWYEGSNKYYADSVKGRFTISRDN |
| | | SKNTLYLQMNSLRAEDTAVYYCARGGSMVRGDYYYGM |
| | | DVWGQGTTVTVSS |
| SEQ ID NO: | VL | AIQLTQSPSSLSASVGDRVTITCRASQGISSALAWYQ |
| 21 | | QKPGKAPKLLIYDASSLESGVPSRFSGSGSGTDFTLT |
| | | ISSLQPEDFATYYCQQFNSYPYTFGQGTKLEIK |

In certain embodiments, the immunomodulator is an inhibitor of an immune checkpoint molecule. In one embodiment, the immunomodulator is an inhibitor of PD-1, PD-L1, PD-L2, CTLA4, TIM3, LAG3, VISTA, BTLA, TIGIT, LAIR1, CD160, 2B4 and/or TGFRbeta. In one embodiment, the inhibitor of an immune checkpoint molecule inhibits PD-1, PD-L1, LAG-3, TIM-3 or CTLA4, or any combination thereof. The term “inhibition” or “inhibitor” includes a reduction in a certain parameter, e.g., an activity, of a given molecule, e.g., an immune checkpoint inhibitor. For example, inhibition of an activity, e.g., a PD-1 or PD-L1 activity, of at least 5%, 10%, 20%, 30%, 40%, 50% or more is included by this term. Thus, inhibition need not be 100%.

Inhibition of an inhibitory molecule can be performed at the DNA, RNA or protein level. In some embodiments, an inhibitory nucleic acid (e.g., a dsRNA, siRNA or shRNA), can be used to inhibit expression of an inhibitory molecule. In other embodiments, the inhibitor of an inhibitory signal is a polypeptide e.g., a soluble ligand (e.g., PD-1-Ig or CTLA-4 Ig), or an antibody or antigen-binding fragment thereof, that binds to the inhibitory molecule; e.g., an antibody or fragment thereof (also referred to herein as “an antibody molecule”) that binds to PD-1, PD-L1, PD-L2, CTLA4, TIM3, LAG3, VISTA, BTLA, TIGIT, LAIR1, CD160, 2B4 and/or TGFR beta, or a combination thereof.

In one embodiment, the antibody molecule is a full antibody or fragment thereof (e.g., a Fab, F(ab′)₂, Fv, or a single chain Fv fragment (scFv)). In yet other embodiments, the antibody molecule has a heavy chain constant region (Fc) selected from, e.g., the heavy chain constant regions of IgG1, IgG2, IgG3, IgG4, IgM, IgA1, IgA2, IgD, and IgE; particularly, selected from, e.g., the heavy chain constant regions of IgG1, IgG2, IgG3, and IgG4, more particularly, the heavy chain constant region of IgG1 or IgG4 (e.g., human IgG1 or IgG4). In one embodiment, the heavy chain constant region is human IgG1 or human IgG4. In one embodiment, the constant region is altered, e.g., mutated, to modify the properties of the antibody molecule (e.g., to increase or decrease one or more of Fc receptor binding, antibody glycosylation, the number of cysteine residues, effector cell function, or complement function).

In certain embodiments, the antibody molecule is in the form of a bispecific or multispecific antibody molecule. In one embodiment, the bispecific antibody molecule has a first binding specificity to PD-1 or PD-L1 and a second binding specificity, e.g., a second binding specificity to TIM-3, LAG-3, or PD-L2. In one embodiment, the bispecific antibody molecule binds to PD-1 or PD-L1 and TIM-3. In another embodiment, the bispecific antibody molecule binds to PD-1 or PD-L1 and LAG-3. In another embodiment, the bispecific antibody molecule binds to PD-1 and PD-L1. In yet another embodiment, the bispecific antibody molecule binds to PD-1 and PD-L2. In another embodiment, the bispecific antibody molecule binds to TIM-3 and LAG-3. Any combination of the aforesaid molecules can be made in a multispecific antibody molecule, e.g., a trispecific antibody that includes a first binding specificity to PD-1 or PD-1, and a second and third binding specificities to two or more of TIM-3, LAG-3, or PD-L2.

In certain embodiments, the immunomodulator is an inhibitor of PD-1, e.g., human PD-1. In another embodiment, the immunomodulator is an inhibitor of PD-L1, e.g., human PD-L1. In one embodiment, the inhibitor of PD-1 or PD-L1 is an antibody molecule to PD-1 or PD-L1. The PD-1 or PD-L1 inhibitor can be administered alone, or in combination with other immunomodulators, e.g., in combination with an inhibitor of LAG-3, TIM-3 or CTLA4. In an exemplary embodiment, the inhibitor of PD-1 or PD-L1, e.g., the anti-PD-1 or PD-L1 antibody molecule, is administered in combination with a LAG-3 inhibitor, e.g., an anti-LAG-3 antibody molecule. In another embodiment, the inhibitor of PD-1 or PD-L1, e.g., the anti-PD-1 or PD-L1 antibody molecule, is administered in combination with a TIM-3 inhibitor, e.g., an anti-TIM-3 antibody molecule. In yet other embodiments, the inhibitor of PD-1 or PD-L1, e.g., the anti-PD-1 antibody molecule, is administered in combination with a LAG-3 inhibitor, e.g., an anti-LAG-3 antibody molecule, and a TIM-3 inhibitor, e.g., an anti-TIM-3 antibody molecule.

Other combinations of immunomodulators with a PD-1 inhibitor (e.g., one or more of PD-L2, CTLA4, TIM3, LAG3, VISTA, BTLA, TIGIT, LAIR1, CD160, 2B4 and/or TGFR) are also within the present disclosure. Any of the antibody molecules known in the art or disclosed herein can be used in the aforesaid combinations of inhibitors of checkpoint molecule.

### PD-1 Inhibitors

In some embodiments, the compounds of Formula (I), or Embodiment 16, 17, or 35, or a pharmaceutically acceptable salt, hydrate, solvate, prodrug, stereoisomer, or tautomer thereof, of the present disclosure are used in combination with a PD-1 inhibitor to treat a disease, e.g., cancer. In some embodiments, the PD-1 inhibitor is selected from PDR001 (Novartis), Nivolumab (Bristol-Myers Squibb), Pembrolizumab (Merck & Co), Pidilizumab (CureTech), MEDI0680 (Medimmune), REGN2810 (Regeneron), TSR-042 (Tesaro), PF-06801591 (Pfizer), BGB-A317 (Beigene), BGB-108 (Beigene), INCSHR1210 (Incyte), or AMP-224 (Amplimmune).

### Exemplary PD-1 Inhibitors

In one embodiment, the PD-1 inhibitor is an anti-PD-1 antibody molecule. In one embodiment, the PD-1 inhibitor is an anti-PD-1 antibody molecule as described in US 2015/0210769, published on Jul. 30, 2015, entitled “Antibody Molecules to PD-1 and Uses Thereof,” incorporated by reference in its entirety.

In one embodiment, the anti-PD-1 antibody molecule comprises at least one, two, three, four, five or six complementarity determining regions (CDRs) (or collectively all of the CDRs) from a heavy and light chain variable region comprising an amino acid sequence shown in Table 3 (e.g., from the heavy and light chain variable region sequences of BAP049-Clone-E or BAP049-Clone-B disclosed in Table 3), or encoded by a nucleotide sequence shown in Table 3. In some embodiments, the CDRs are according to the Kabat definition (e.g., as set out in Table 3). In some embodiments, the CDRs are according to the Chothia definition (e.g., as set out in Table 3). In some embodiments, the CDRs are according to the combined CDR definitions of both Kabat and Chothia (e.g., as set out in Table 3). In one embodiment, the combination of Kabat and Chothia CDR of VH CDR1 comprises the amino acid sequence GYTFTTYWMH (SEQ ID NO: 213). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 3, or encoded by a nucleotide sequence shown in Table 3.

In one embodiment, the anti-PD-1 antibody molecule comprises a heavy chain variable region (VH) comprising a VHCDR1 amino acid sequence of SEQ ID NO: 22, a VHCDR2 amino acid sequence of SEQ ID NO: 23, and a VHCDR3 amino acid sequence of SEQ ID NO: 24; and a light chain variable region (VL) comprising a VLCDR1 amino acid sequence of SEQ ID NO: 31, a VLCDR2 amino acid sequence of SEQ ID NO: 32, and a VLCDR3 amino acid sequence of SEQ ID NO: 286, each disclosed in Table 3.

In one embodiment, the antibody molecule comprises a VH comprising a VHCDR1 encoded by the nucleotide sequence of SEQ ID NO: 45, a VHCDR2 encoded by the nucleotide sequence of SEQ ID NO: 46, and a VHCDR3 encoded by the nucleotide sequence of SEQ ID NO: 47; and a VL comprising a VLCDR1 encoded by the nucleotide sequence of SEQ ID NO: 50, a VLCDR2 encoded by the nucleotide sequence of SEQ ID NO: 51, and a VLCDR3 encoded by the nucleotide sequence of SEQ ID NO: 52, each disclosed in Table 3.

In one embodiment, the anti-PD-1 antibody molecule comprises a VH comprising the amino acid sequence of SEQ ID NO: 27, or an amino acid sequence at least 85%, 90%, 95%, or 99% identical or higher to SEQ ID NO: 27. In one embodiment, the anti-PD-1 antibody molecule comprises a VL comprising the amino acid sequence of SEQ ID NO: 41, or an amino acid sequence at least 85%, 90%, 95%, or 99% identical or higher to SEQ ID NO: 41. In one embodiment, the anti-PD-1 antibody molecule comprises a VL comprising the amino acid sequence of SEQ ID NO: 37, or an amino acid sequence at least 85%, 90%, 95%, or 99% identical or higher to SEQ ID NO: 37. In one embodiment, the anti-PD-1 antibody molecule comprises a VH comprising the amino acid sequence of SEQ ID NO: 27 and a VL comprising the amino acid sequence of SEQ ID NO: 41. In one embodiment, the anti-PD-1 antibody molecule comprises a VH comprising the amino acid sequence of SEQ ID NO: 27 and a VL comprising the amino acid sequence of SEQ ID NO: 37.

In one embodiment, the antibody molecule comprises a VH encoded by the nucleotide sequence of SEQ ID NO: 28, or a nucleotide sequence at least 85%, 90%, 95%, or 99% identical or higher to SEQ ID NO: 28. In one embodiment, the antibody molecule comprises a VL encoded by the nucleotide sequence of SEQ ID NO: 42 or 38, or a nucleotide sequence at least 85%, 90%, 95%, or 99% identical or higher to SEQ ID NO: 42 or 38. In one embodiment, the antibody molecule comprises a VH encoded by the nucleotide sequence of SEQ ID NO: 28 and a VL encoded by the nucleotide sequence of SEQ ID NO: 42 or 38.

In one embodiment, the anti-PD-1 antibody molecule comprises a heavy chain comprising the amino acid sequence of SEQ ID NO: 29, or an amino acid sequence at least 85%, 90%, 95%, or 99% identical or higher to SEQ ID NO: 29. In one embodiment, the anti-PD-1 antibody molecule comprises a light chain comprising the amino acid sequence of SEQ ID NO: 43, or an amino acid sequence at least 85%, 90%, 95%, or 99% identical or higher to SEQ ID NO: 43. In one embodiment, the anti-PD-1 antibody molecule comprises a light chain comprising the amino acid sequence of SEQ ID NO: 39, or an amino acid sequence at least 85%, 90%, 95%, or 99% identical or higher to SEQ ID NO: 39. In one embodiment, the anti-PD-1 antibody molecule comprises a heavy chain comprising the amino acid sequence of SEQ ID NO: 29 and a light chain comprising the amino acid sequence of SEQ ID NO: 43. In one embodiment, the anti-PD-1 antibody molecule comprises a heavy chain comprising the amino acid sequence of SEQ ID NO: 29 and a light chain comprising the amino acid sequence of SEQ ID NO: 39.

In one embodiment, the antibody molecule comprises a heavy chain encoded by the nucleotide sequence of SEQ ID NO: 30, or a nucleotide sequence at least 85%, 90%, 95%, or 99% identical or higher to SEQ ID NO: 30. In one embodiment, the antibody molecule comprises a light chain encoded by the nucleotide sequence of SEQ ID NO: 44 or 40, or a nucleotide sequence at least 85%, 90%, 95%, or 99% identical or higher to SEQ ID NO: 44 or 40. In one embodiment, the antibody molecule comprises a heavy chain encoded by the nucleotide sequence of SEQ ID NO: 30 and a light chain encoded by the nucleotide sequence of SEQ ID NO: 44 or 40.

The antibody molecules described herein can be made by vectors, host cells, and methods described in US 2015/0210769, incorporated by reference in its entirety.

TABLE 3
Amino acid and nucleotide sequences of exemplary
anti-PD-1 antibody molecules

| | | |
|---|---|---|
| BAP049-Clone-B HC | | |
| SEQ ID NO: 22 (Kabat) | HCDR1 | TYWMH |
| SEQ ID NO: 23 (Kabat) | HCDR2 | NIYPGTGGSNFDEKFKN |
| SEQ ID NO: 24 (Kabat) | HCDR3 | WTTGTGAY |
| SEQ ID NO: 25 | HCDR1 | GYTFTTY |
| (Chothia) | | |
| SEQ ID NO: 26 | HCDR2 | YPGTGG |
| (Chothia) | | |
| SEQ ID NO: 24 | HCDR3 | WTTGTGAY |
| (Chothia) | | |
| SEQ ID NO: 27 | VH | EVQLVQSGAEVKKPGESLRISCKGSGYTFTTYWMHWVRQA |
| | | TGQGLEWMGNIYPGTGGSNFDEKFKNRVTITADKSTSTAY |
| | | MELSSLRSEDTAVYYCTRWTTGTGAYWGQGTTVTVSS |
| SEQ ID NO: 28 | DNA | GAGGTGCAGCTGGTGCAGTCAGGCGCCGAAGTGAAGAAG |
| | VH | CCCGGCGAGTCACTGAGAATTAGCTGTAAAGGTTCAGGC |
| | | TACACCTTCACTACCTACTGGATGCACTGGGTCCGCCAGG |
| | | CTACCGGTCAAGGCCTCGAGTGGATGGGTAATATCTACC |
| | | CCGGCACCGGCGGCTCTAACTTCGACGAGAAGTTTAAGA |
| | | ATAGAGTGACTATCACCGCCGATAAGTCTACTAGCACCG |
| | | CCTATATGGAACTGTCTAGCCTGAGATCAGAGGACACCG |
| | | CCGTCTACTACTGCACTAGGTGGACTACCGGCACAGGCG |
| | | CCTACTGGGGTCAAGGCACTACCGTGACCGTGTCTAGC |
| SEQ ID NO: 29 | Heavy | EVQLVQSGAEVKKPGESLRISCKGSGYTFTTYWMHWVRQA |
| | chain | TGQGLEWMGNIYPGTGGSNFDEKFKNRVTITADKSTSTAY |
| | | MELSSLRSEDTAVYYCTRWTTGTGAYWGQGTTVTVSSAST |
| | | KGPSVFPLAPCSRSTSESTAALGCLVKDYFPEPVTVSWNSGA |
| | | LTSGVHTFPAVLQSSGLYSLSSVVTVPSSSLGTKTYTCNVDH |
| | | KPSNTKVDKRVESKYGPPCPPCPAPEFLGGPSVFLFPPKPKD |
| | | TLMISRTPEVTCVVVDVSQEDPEVQFNWYVDGVEVHNAKT |
| | | KPREEQFNSTYRVVSVLTVLHQDWLNGKEYKCKVSNKGLP |
| | | SSIEKTISKAKGQPREPQVYTLPPSQEEMTKNQVSLTCLVKG |
| | | FYPSDIAVEWESNGQPENNYKTTPPVLDSDGSFFLYSRLTVD |
| | | KSRWQEGNVFSCSVMHEALHNHYTQKSLSLSLG |
| SEQ ID NO: 30 | DNA | GAGGTGCAGCTGGTGCAGTCAGGCGCCGAAGTGAAGAAG |
| | heavy | CCCGGCGAGTCACTGAGAATTAGCTGTAAAGGTTCAGGC |
| | chain | TACACCTTCACTACCTACTGGATGCACTGGGTCCGCCAGG |
| | | CTACCGGTCAAGGCCTCGAGTGGATGGGTAATATCTACC |
| | | CCGGCACCGGCGGCTCTAACTTCGACGAGAAGTTTAAGA |
| | | ATAGAGTGACTATCACCGCCGATAAGTCTACTAGCACCG |
| | | CCTATATGGAACTGTCTAGCCTGAGATCAGAGGACACCG |
| | | CCGTCTACTACTGCACTAGGTGGACTACCGGCACAGGCG |
| | | CCTACTGGGGTCAAGGCACTACCGTGACCGTGTCTAGCG |
| | | CTAGCACTAAGGGCCCGTCCGTGTTCCCCCTGGCACCTTG |
| | | TAGCCGGAGCACTAGCGAATCCACCGCTGCCCTCGGCTG |
| | | CCTGGTCAAGGATTACTTCCCGGAGCCCGTGACCGTGTCC |
| | | TGGAACAGCGGAGCCCTGACCTCCGGAGTGCACACCTTC |
| | | CCCGCTGTGCTGCAGAGCTCCGGGCTGTACTCGCTGTCGT |
| | | CGGTGGTCACGGTGCCTTCATCTAGCCTGGGTACCAAGAC |
| | | CTACACTTGCAACGTGGACCACAAGCCTTCCAACACTAA |
| | | GGTGGACAAGCGCGTCGAATCGAAGTACGGCCCACCGTG |
| | | CCCGCCTTGTCCCGCGCCGGAGTTCCTCGGCGGTCCCTCG |
| | | GTCTTTCTGTTCCCACCGAAGCCCAAGGACACTTTGATGA |
| | | TTTCCCGCACCCCTGAAGTGACATGCGTGGTCGTGGACGT |
| | | GTCACAGGAAGATCCGGAGGTGCAGTTCAATTGGTACGT |
| | | GGATGGCGTCGAGGTGCACAACGCCAAAACCAAGCCGAG |
| | | GGAGGAGCAGTTCAACTCCACTTACCGCGTCGTGTCCGTG |
| | | CTGACGGTGCTGCATCAGGACTGGCTGAACGGGAAGGAG |
| | | TACAAGTGCAAAGTGTCCAACAAGGGACTTCCTAGCTCA |
| | | ATCGAAAAGACCATCTCGAAAGCCAAGGGACAGCCCCGG |
| | | GAACCCCAAGTGTATACCCTGCCACCGAGCCAGGAAGAA |
| | | ATGACTAAGAACCAAGTCTCATTGACTTGCCTTGTGAAGG |
| | | GCTTCTACCCATCGGATATCGCCGTGGAATGGGAGTCCA |
| | | ACGGCCAGCCGGAAAACAACTACAAGACCACCCCTCCGG |
| | | TGCTGGACTCAGACGGATCCTTCTTCCTCTACTCGCGGCT |
| | | GACCGTGGATAAGAGCAGATGGCAGGAGGGAAATGTGTT |
| | | CAGCTGTTCTGTGATGCATGAAGCCCTGCACAACCACTAC |
| | | ACTCAGAAGTCCCTGTCCCTCTCCCTGGGA |
| BAP049-Clone-B LC | | |
| SEQ ID NO: 31 (Kabat) | LCDR1 | KSSQSLLDSGNQKNFLT |
| SEQ ID NO: 32 (Kabat) | LCDR2 | WASTRES |
| SEQ ID NO: 286 | LCDR3 | QNDYSYPYT |
| (Kabat) | | |
| SEQ ID NO: 34 | LCDR1 | SQSLLDSGNQKNF |
| (Chothia) | | |
| SEQ ID NO: 35 | LCDR2 | WAS |
| (Chothia) | | |
| SEQ ID NO: 36 | LCDR3 | DYSYPY |
| (Chothia) | | |
| SEQ ID NO: 37 | VL | EIVLTQSPATLSLSPGERATLSCKSSQSLLDSGNQKNFLTWY |
| | | QQKPGKAPKLLIYWASTRESGVPSRFSGSGSGTDFTFTISSLQ |
| | | PEDIATYYCQNDYSYPYTFGQGTKVEIK |
| SEQ ID NO: 38 | DNA | GAGATCGTCCTGACTCAGTCACCCGCTACCCTGAGCCTGA |
| | VL | GCCCTGGCGAGCGGGCTACACTGAGCTGTAAATCTAGTC |
| | | AGTCACTGCTGGATAGCGGTAATCAGAAGAACTTCCTGA |
| | | CCTGGTATCAGCAGAAGCCCGGTAAAGCCCCTAAGCTGC |
| | | TGATCTACTGGGCCTCTACTAGAGAATCAGGCGTGCCCTC |
| | | TAGGTTTAGCGGTAGCGGTAGTGGCACCGACTTCACCTTC |
| | | ACTATCTCTAGCCTGCAGCCCGAGGATATCGCTACCTACT |
| | | ACTGTCAGAACGACTATAGCTACCCCTACACCTTCGGTCA |
| | | AGGCACTAAGGTCGAGATTAAG |
| SEQ ID NO: 39 | Light | EIVLTQSPATLSLSPGERATLSCKSSQSLLDSGNQKNFLTWY |
| | chain | QQKPGKAPKLLIYWASTRESGVPSRFSGSGSGTDFTFTISSLQ |
| | | PEDIATYYCQNDYSYPYTFGQGTKVEIKRTVAAPSVFIFPPS |
| | | DEQLKSGTASVVCLLNNFYPREAKVQWKVDNALQSGNSQE |
| | | SVTEQDSKDSTYSLSSTLTLSKADYEKHKVYACEVTHQGLS |
| | | SPVTKSFNRGEC |
| SEQ ID NO: 40 | DNA | GAGATCGTCCTGACTCAGTCACCCGCTACCCTGAGCCTGA |
| | light | GCCCTGGCGAGCGGGCTACACTGAGCTGTAAATCTAGTC |
| | chain | AGTCACTGCTGGATAGCGGTAATCAGAAGAACTTCCTGA |
| | | CCTGGTATCAGCAGAAGCCCGGTAAAGCCCCTAAGCTGC |
| | | TGATCTACTGGGCCTCTACTAGAGAATCAGGCGTGCCCTC |
| | | TAGGTTTAGCGGTAGCGGTAGTGGCACCGACTTCACCTTC |
| | | ACTATCTCTAGCCTGCAGCCCGAGGATATCGCTACCTACT |
| | | ACTGTCAGAACGACTATAGCTACCCCTACACCTTCGGTCA |
| | | AGGCACTAAGGTCGAGATTAAGCGTACGGTGGCCGCTCC |
| | | CAGCGTGTTCATCTTCCCCCCCAGCGACGAGCAGCTGAA |
| | | GAGCGGCACCGCCAGCGTGGTGTGCCTGCTGAACAACTT |
| | | CTACCCCCGGGAGGCCAAGGTGCAGTGGAAGGTGGACAA |
| | | CGCCCTGCAGAGCGGCAACAGCCAGGAGAGCGTCACCGA |
| | | GCAGGACAGCAAGGACTCCACCTACAGCCTGAGCAGCAC |
| | | CCTGACCCTGAGCAAGGCCGACTACGAGAAGCATAAGGT |
| | | GTACGCCTGCGAGGTGACCCACCAGGGCCTGTCCAGCCC |
| | | CGTGACCAAGAGCTTCAACAGGGGCGAGTGC |
| BAP049-Clone-E HC | | |
| SEQ ID NO: 22 (Kabat) | HCDR1 | TYWMH |
| SEQ ID NO: 23 (Kabat) | HCDR2 | NIYPGTGGSNFDEKFKN |
| SEQ ID NO: 24 (Kabat) | HCDR3 | WTTGTGAY |
| SEQ ID NO: 25 | HCDR1 | GYTFTTY |
| (Chothia) | | |
| SEQ ID NO: 26 | HCDR2 | YPGTGG |
| (Chothia) | | |
| SEQ ID NO: 24 | HCDR3 | WTTGTGAY |
| (Chothia) | | |
| SEQ ID NO: 27 | VH | EVQLVQSGAEVKKPGESLRISCKGSGYTFTTYWMHWVRQA |
| | | TGQGLEWMGNIYPGTGGSNFDEKFKNRVTITADKSTSTAY |
| | | MELSSLRSEDTAVYYCTRWTTGTGAYWGQGTTVTVSS |
| SEQ ID NO: 28 | DNA | GAGGTGCAGCTGGTGCAGTCAGGCGCCGAAGTGAAGAAG |
| | VH | CCCGGCGAGTCACTGAGAATTAGCTGTAAAGGTTCAGGC |
| | | TACACCTTCACTACCTACTGGATGCACTGGGTCCGCCAGG |
| | | CTACCGGTCAAGGCCTCGAGTGGATGGGTAATATCTACC |
| | | CCGGCACCGGCGGCTCTAACTTCGACGAGAAGTTTAAGA |
| | | ATAGAGTGACTATCACCGCCGATAAGTCTACTAGCACCG |
| | | CCTATATGGAACTGTCTAGCCTGAGATCAGAGGACACCG |
| | | CCGTCTACTACTGCACTAGGTGGACTACCGGCACAGGCG |
| | | CCTACTGGGGTCAAGGCACTACCGTGACCGTGTCTAGC |
| SEQ ID NO: 29 | Heavy | EVQLVQSGAEVKKPGESLRISCKGSGYTFTTYWMHWVRQA |
| | chain | TGQGLEWMGNIYPGTGGSNFDEKFKNRVTITADKSTSTAY |
| | | MELSSLRSEDTAVYYCTRWTTGTGAYWGQGTTVTVSSAST |
| | | KGPSVFPLAPCSRSTSESTAALGCLVKDYFPEPVTVSWNSGA |
| | | LTSGVHTFPAVLQSSGLYSLSSVVTVPSSSLGTKTYTCNVDH |
| | | KPSNTKVDKRVESKYGPPCPPCPAPEFLGGPSVFLFPPKPKD |
| | | TLMISRTPEVTCVVVDVSQEDPEVQFNWYVDGVEVHNAKT |
| | | KPREEQFNSTYRVVSVLTVLHQDWLNGKEYKCKVSNKGLP |
| | | SSIEKTISKAKGQPREPQVYTLPPSQEEMTKNQVSLTCLVKG |
| | | FYPSDIAVEWESNGQPENNYKTTPPVLDSDGSFFLYSRLTVD |
| | | KSRWQEGNVFSCSVMHEALHNHYTQKSLSLSLG |
| SEQ ID NO: 30 | DNA | GAGGTGCAGCTGGTGCAGTCAGGCGCCGAAGTGAAGAAG |
| | heavy | CCCGGCGAGTCACTGAGAATTAGCTGTAAAGGTTCAGGC |
| | chain | TACACCTTCACTACCTACTGGATGCACTGGGTCCGCCAGG |
| | | CTACCGGTCAAGGCCTCGAGTGGATGGGTAATATCTACC |
| | | CCGGCACCGGCGGCTCTAACTTCGACGAGAAGTTTAAGA |
| | | ATAGAGTGACTATCACCGCCGATAAGTCTACTAGCACCG |
| | | CCTATATGGAACTGTCTAGCCTGAGATCAGAGGACACCG |
| | | CCGTCTACTACTGCACTAGGTGGACTACCGGCACAGGCG |
| | | CCTACTGGGGTCAAGGCACTACCGTGACCGTGTCTAGCG |
| | | CTAGCACTAAGGGCCCGTCCGTGTTCCCCCTGGCACCTTG |
| | | TAGCCGGAGCACTAGCGAATCCACCGCTGCCCTCGGCTG |
| | | CCTGGTCAAGGATTACTTCCCGGAGCCCGTGACCGTGTCC |
| | | TGGAACAGCGGAGCCCTGACCTCCGGAGTGCACACCTTC |
| | | CCCGCTGTGCTGCAGAGCTCCGGGCTGTACTCGCTGTCGT |
| | | CGGTGGTCACGGTGCCTTCATCTAGCCTGGGTACCAAGAC |
| | | CTACACTTGCAACGTGGACCACAAGCCTTCCAACACTAA |
| | | GGTGGACAAGCGCGTCGAATCGAAGTACGGCCCACCGTG |
| | | CCCGCCTTGTCCCGCGCCGGAGTTCCTCGGCGGTCCCTCG |
| | | GTCTTTCTGTTCCCACCGAAGCCCAAGGACACTTTGATGA |
| | | TTTCCCGCACCCCTGAAGTGACATGCGTGGTCGTGGACGT |
| | | GTCACAGGAAGATCCGGAGGTGCAGTTCAATTGGTACGT |
| | | GGATGGCGTCGAGGTGCACAACGCCAAAACCAAGCCGAG |
| | | GGAGGAGCAGTTCAACTCCACTTACCGCGTCGTGTCCGTG |
| | | CTGACGGTGCTGCATCAGGACTGGCTGAACGGGAAGGAG |
| | | TACAAGTGCAAAGTGTCCAACAAGGGACTTCCTAGCTCA |
| | | ATCGAAAAGACCATCTCGAAAGCCAAGGGACAGCCCCGG |
| | | GAACCCCAAGTGTATACCCTGCCACCGAGCCAGGAAGAA |
| | | ATGACTAAGAACCAAGTCTCATTGACTTGCCTTGTGAAGG |
| | | GCTTCTACCCATCGGATATCGCCGTGGAATGGGAGTCCA |
| | | ACGGCCAGCCGGAAAACAACTACAAGACCACCCCTCCGG |
| | | TGCTGGACTCAGACGGATCCTTCTTCCTCTACTCGCGGCT |
| | | GACCGTGGATAAGAGCAGATGGCAGGAGGGAAATGTGTT |
| | | CAGCTGTTCTGTGATGCATGAAGCCCTGCACAACCACTAC |
| | | ACTCAGAAGTCCCTGTCCCTCTCCCTGGGA |
| BAP049-Clone-E LC | | |
| SEQ ID NO: 31 (Kabat) | LCDR1 | KSSQSLLDSGNQKNFLT |
| SEQ ID NO: 32 (Kabat) | LCDR2 | WASTRES |
| SEQ ID NO: 286 | LCDR3 | QNDYSYPYT |
| (Kabat) | | |
| SEQ ID NO: 34 | LCDR1 | SQSLLDSGNQKNF |
| (Chothia) | | |
| SEQ ID NO: 35 | LCDR2 | WAS |
| (Chothia) | | |
| SEQ ID NO: 36 | LCDR3 | DYSYPY |
| (Chothia) | | |
| SEQ ID NO: 41 | VL | EIVLTQSPATLSLSPGERATLSCKSSQSLLDSGNQKNFLTWY |
| | | QQKPGQAPRLLIYWASTRESGVPSRFSGSGSGTDFTFTISSLE |
| | | AEDAATYYCQNDYSYPYTFGQGTKVEIK |
| SEQ ID NO: 42 | DNA | GAGATCGTCCTGACTCAGTCACCCGCTACCCTGAGCCTGA |
| | VL | GCCCTGGCGAGCGGGCTACACTGAGCTGTAAATCTAGTC |
| | | AGTCACTGCTGGATAGCGGTAATCAGAAGAACTTCCTGA |
| | | CCTGGTATCAGCAGAAGCCCGGTCAAGCCCCTAGACTGC |
| | | TGATCTACTGGGCCTCTACTAGAGAATCAGGCGTGCCCTC |
| | | TAGGTTTAGCGGTAGCGGTAGTGGCACCGACTTCACCTTC |
| | | ACTATCTCTAGCCTGGAAGCCGAGGACGCCGCTACCTACT |
| | | ACTGTCAGAACGACTATAGCTACCCCTACACCTTCGGTCA |
| | | AGGCACTAAGGTCGAGATTAAG |
| SEQ ID NO: 43 | Light | EIVLTQSPATLSLSPGERATLSCKSSQSLLDSGNQKNFLTWY |
| | chain | QQKPGQAPRLLIYWASTRESGVPSRFSGSGSGTDFTFTISSLE |
| | | AEDAATYYCQNDYSYPYTFGQGTKVEIKRTVAAPSVFIFPPS |
| | | DEQLKSGTASVVCLLNNFYPREAKVQWKVDNALQSGNSQE |
| | | SVTEQDSKDSTYSLSSTLTLSKADYEKHKVYACEVTHQGLS |
| | | SPVTKSFNRGEC |
| SEQ ID NO: 44 | DNA | GAGATCGTCCTGACTCAGTCACCCGCTACCCTGAGCCTGA |
| | light | GCCCTGGCGAGCGGGCTACACTGAGCTGTAAATCTAGTC |
| | chain | AGTCACTGCTGGATAGCGGTAATCAGAAGAACTTCCTGA |
| | | CCTGGTATCAGCAGAAGCCCGGTCAAGCCCCTAGACTGC |
| | | TGATCTACTGGGCCTCTACTAGAGAATCAGGCGTGCCCTC |
| | | TAGGTTTAGCGGTAGCGGTAGTGGCACCGACTTCACCTTC |
| | | ACTATCTCTAGCCTGGAAGCCGAGGACGCCGCTACCTACT |
| | | ACTGTCAGAACGACTATAGCTACCCCTACACCTTCGGTCA |
| | | AGGCACTAAGGTCGAGATTAAGCGTACGGTGGCCGCTCC |
| | | CAGCGTGTTCATCTTCCCCCCCAGCGACGAGCAGCTGAA |
| | | GAGCGGCACCGCCAGCGTGGTGTGCCTGCTGAACAACTT |
| | | CTACCCCCGGGAGGCCAAGGTGCAGTGGAAGGTGGACAA |
| | | CGCCCTGCAGAGCGGCAACAGCCAGGAGAGCGTCACCGA |
| | | GCAGGACAGCAAGGACTCCACCTACAGCCTGAGCAGCAC |
| | | CCTGACCCTGAGCAAGGCCGACTACGAGAAGCATAAGGT |
| | | GTACGCCTGCGAGGTGACCCACCAGGGCCTGTCCAGCCC |
| | | CGTGACCAAGAGCTTCAACAGGGGCGAGTGC |
| BAP049-Clone-B HC | | |
| SEQ ID NO: 45 (Kabat) | HCDR1 | ACCTACTGGATGCAC |
| SEQ ID NO: 46 (Kabat) | HCDR2 | AATATCTACCCCGGCACCGGCGGCTCTAACTTCGACGAG |
| | | AAGTTTAAGAAT |
| SEQ ID NO: 47 (Kabat) | HCDR3 | TGGACTACCGGCACAGGCGCCTAC |
| SEQ ID NO: 48 | HCDR1 | GGCTACACCTTCACTACCTAC |
| (Chothia) | | |
| SEQ ID NO: 49 | HCDR2 | TACCCCGGCACCGGCGGC |
| (Chothia) | | |
| SEQ ID NO: 47 | HCDR3 | TGGACTACCGGCACAGGCGCCTAC |
| (Chothia) | | |
| BAP049-Clone-B LC | | |
| SEQ ID NO: 50 (Kabat) | LCDR1 | AAATCTAGTCAGTCACTGCTGGATAGCGGTAATCAGAAG |
| | | AACTTCCTGACC |
| SEQ ID NO: 51 (Kabat) | LCDR2 | TGGGCCTCTACTAGAGAATCA |
| SEQ ID NO: 52 (Kabat) | LCDR3 | CAGAACGACTATAGCTACCCCTACACC |
| SEQ ID NO: 53 | LCDR1 | AGTCAGTCACTGCTGGATAGCGGTAATCAGAAGAACTTC |
| (Chothia) | | |
| SEQ ID NO: 54 | LCDR2 | TGGGCCTCT |
| (Chothia) | | |
| SEQ ID NO: 55 | LCDR3 | GACTATAGCTACCCCTAC |
| (Chothia) | | |
| BAP049-Clone-E HC | | |
| SEQ ID NO: 45 (Kabat) | HCDR1 | ACCTACTGGATGCAC |
| SEQ ID NO: 46 (Kabat) | HCDR2 | AATATCTACCCCGGCACCGGCGGCTCTAACTTCGACGAG |
| | | AAGTTTAAGAAT |
| SEQ ID NO: 47 (Kabat) | HCDR3 | TGGACTACCGGCACAGGCGCCTAC |
| SEQ ID NO: 48 | HCDR1 | GGCTACACCTTCACTACCTAC |
| (Chothia) | | |
| SEQ ID NO: 49 | HCDR2 | TACCCCGGCACCGGCGGC |
| (Chothia) | | |
| SEQ ID NO: 47 | HCDR3 | TGGACTACCGGCACAGGCGCCTAC |
| (Chothia) | | |
| BAP049-Clone-E LC | | |
| SEQ ID NO: 50 (Kabat) | LCDR1 | AAATCTAGTCAGTCACTGCTGGATAGCGGTAATCAGAAG |
| | | AACTTCCTGACC |
| SEQ ID NO: 51 (Kabat) | LCDR2 | TGGGCCTCTACTAGAGAATCA |
| SEQ ID NO: 52 (Kabat) | LCDR3 | CAGAACGACTATAGCTACCCCTACACC |
| SEQ ID NO: 53 | LCDR1 | AGTCAGTCACTGCTGGATAGCGGTAATCAGAAGAACTTC |
| (Chothia) | | |
| SEQ ID NO: 54 | LCDR2 | TGGGCCTCT |
| (Chothia) | | |
| SEQ ID NO: 55 | LCDR3 | GACTATAGCTACCCCTAC |
| (Chothia) | | |

### Other Exemplary PD-1 Inhibitors

In some embodiments, the anti-PD-1 antibody is Nivolumab (CAS Registry Number: 946414-94-4). Alternative names for Nivolumab include MDX-1106, MDX-1106-04, ONO-4538, BMS-936558 or OPDIVO®. Nivolumab is a fully human IgG4 monoclonal antibody, which specifically blocks PD1. Nivolumab (clone 5C4) and other human monoclonal antibodies that specifically bind to PD1 are disclosed in U.S. Pat. No. 8,008,449 and PCT Publication No. WO2006/121168, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of Nivolumab, e.g., as disclosed in Table 4.

In other embodiments, the anti-PD-1 antibody is Pembrolizumab. Pembrolizumab (Trade name KEYTRUDA formerly Lambrolizumab, also known as Merck 3745, MK-3475 or SCH-900475) is a humanized IgG4 monoclonal antibody that binds to PD1. Pembrolizumab is disclosed, e.g., in Hamid, O. et al. (2013) New England Journal of Medicine 369 (2): 134-44, PCT Publication No. WO2009/114335, and U.S. Pat. No. 8,354,509, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of Pembrolizumab, e.g., as disclosed in Table 4.

In some embodiments, the anti-PD-1 antibody is Pidilizumab. Pidilizumab (CT-011; Cure Tech) is a humanized IgG1k monoclonal antibody that binds to PD1. Pidilizumab and other humanized anti-PD-1 monoclonal antibodies are disclosed in PCT Publication No. WO2009/101611, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of Pidilizumab, e.g., as disclosed in Table 4.

Other anti-PD1 antibodies are disclosed in U.S. Pat. No. 8,609,089, US Publication No. 2010028330, and/or US Publication No. 20120114649, incorporated by reference in their entirety. Other anti-PD1 antibodies include AMP 514 (Amplimmune).

In one embodiment, the anti-PD-1 antibody molecule is MEDI0680 (Medimmune), also known as AMP-514. MED10680 and other anti-PD-1 antibodies are disclosed in U.S. Pat. No. 9,205,148 and WO 2012/145493, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of MEDI0680.

In one embodiment, the anti-PD-1 antibody molecule is REGN2810 (Regeneron). In one embodiment, the anti-PD-1 antibody molecule comprises one or more of the CDR sequences (or collectively all of the CDR sequences), the heavy chain or light chain variable region sequence, or the heavy chain or light chain sequence of REGN2810.

In one embodiment, the anti-PD-1 antibody molecule is PF-06801591 (Pfizer). In one embodiment, the anti-PD-1 antibody molecule comprises one or more of the CDR sequences (or collectively all of the CDR sequences), the heavy chain or light chain variable region sequence, or the heavy chain or light chain sequence of PF-06801591.

In one embodiment, the anti-PD-1 antibody molecule is BGB-A317 or BGB-108 (Beigene). In one embodiment, the anti-PD-1 antibody molecule comprises one or more of the CDR sequences (or collectively all of the CDR sequences), the heavy chain or light chain variable region sequence, or the heavy chain or light chain sequence of BGB-A317 or BGB-108.

In one embodiment, the anti-PD-1 antibody molecule is INCSHR1210 (Incyte), also known as INCSHR01210 or SHR-1210. In one embodiment, the anti-PD-1 antibody molecule comprises one or more of the CDR sequences (or collectively all of the CDR sequences), the heavy chain or light chain variable region sequence, or the heavy chain or light chain sequence of INCSHR1210.

In one embodiment, the anti-PD-1 antibody molecule is TSR-042 (Tesaro), also known as ANB011. In one embodiment, the anti-PD-1 antibody molecule comprises one or more of the CDR sequences (or collectively all of the CDR sequences), the heavy chain or light chain variable region sequence, or the heavy chain or light chain sequence of TSR-042.

Further known anti-PD-1 antibodies include those described, e.g., in WO 2015/112800, WO 2016/092419, WO 2015/085847, WO 2014/179664, WO 2014/194302, WO 2014/209804, WO 2015/200119, U.S. Pat. Nos. 8,735,553, 7,488,802, 8,927,697, 8,993,731, and 9,102,727, incorporated by reference in their entirety.

In one embodiment, the anti-PD-1 antibody is an antibody that competes for binding with, and/or binds to the same epitope on PD-1 as, one of the anti-PD-1 antibodies described herein.

In one embodiment, the PD-1 inhibitor is a peptide that inhibits the PD-1 signaling pathway, e.g., as described in U.S. Pat. No. 8,907,053, incorporated by reference in its entirety. In some embodiments, the PD-1 inhibitor is an immunoadhesin (e.g., an immunoadhesin comprising an extracellular or PD-1 binding portion of PD-L1 or PD-L2 fused to a constant region (e.g., an Fc region of an immunoglobulin sequence). In some embodiments, the PD-1 inhibitor is AMP-224 (B7-DCIg (Amplimmune), e.g., disclosed in WO 2010/027827 and WO 2011/066342, incorporated by reference in their entirety).

TABLE 4
Amino acid sequences of other exemplary
anti-PD-1 antibody molecules

| | | |
|---|---|---|
| Nivolumab | | |
| SEQ ID NO: 56 | Heavy | QVQLVESGGGVVQPGRSLRLDCKASGITFSNSGMHWVRQAPGKG |
| | chain | LEWVAVIWYDGSKRYYADSVKGRFTISRDNSKNTLFLQMNSLRA |
| | | EDTAVYYCATNDDYWGQGTLVTVSSASTKGPSVFPLAPCSRSTSE |
| | | STAALGCLVKDYFPEPVTVSWNSGALTSGVHTFPAVLQSSGLYSL |
| | | SSVVTVPSSSLGTKTYTCNVDHKPSNTKVDKRVESKYGPPCPPCP |
| | | APEFLGGPSVFLFPPKPKDTLMISRTPEVTCVVVDVSQEDPEVQFN |
| | | WYVDGVEVHNAKTKPREEQFNSTYRVVSVLTVLHQDWLNGKEY |
| | | KCKVSNKGLPSSIEKTISKAKGQPREPQVYTLPPSQEEMTKNQVSL |
| | | TCLVKGFYPSDIAVEWESNGQPENNYKTTPPVLDSDGSFFLYSRL |
| | | TVDKSRWQEGNVFSCSVMHEALHNHYTQKSLSLSLGK |
| SEQ ID NO: 57 | Light | EIVLTQSPATLSLSPGERATLSCRASQSVSSYLAWYQQKPGQAPRL |
| | chain | LIYDASNRATGIPARFSGSGSGTDFTLTISSLEPEDFAVYYCQQSSN |
| | | WPRTFGQGTKVEIKRTVAAPSVFIFPPSDEQLKSGTASVVCLLNNF |
| | | YPREAKVQWKVDNALQSGNSQESVTEQDSKDSTYSLSSTLTLSK |
| | | ADYEKHKVYACEVTHQGLSSPVTKSFNRGEC |
| Pembrolizumab | | |
| SEQ ID NO: 58 | Heavy | QVQLVQSGVEVKKPGASVKVSCKASGYTFTNYYMYWVRQAPG |
| | chain | QGLEWMGGINPSNGGTNFNEKFKNRVTLTTDSSTTTAYMELKSL |
| | | QFDDTAVYYCARRDYRFDMGFDYWGQGTTVTVSSASTKGPSVF |
| | | PLAPCSRSTSESTAALGCLVKDYFPEPVTVSWNSGALTSGVHTFP |
| | | AVLQSSGLYSLSSVVTVPSSSLGTKTYTCNVDHKPSNTKVDKRVE |
| | | SKYGPPCPPCPAPEFLGGPSVFLFPPKPKDTLMISRTPEVTCVVVD |
| | | VSQEDPEVQFNWYVDGVEVHNAKTKPREEQFNSTYRVVSVLTVL |
| | | HQDWLNGKEYKCKVSNKGLPSSIEKTISKAKGQPREPQVYTLPPS |
| | | QEEMTKNQVSLTCLVKGFYPSDIAVEWESNGQPENNYKTTPPVL |
| | | DSDGSFFLYSRLTVDKSRWQEGNVFSCSVMHEALHNHYTQKSLS |
| | | LSLGK |
| SEQ ID NO: 59 | Light | EIVLTQSPATLSLSPGERATLSCRASKGVSTSGYSYLHWYQQKPG |
| | chain | QAPRLLIYLASYLESGVPARFSGSGSGTDFTLTISSLEPEDFAVYYC |
| | | QHSRDLPLTFGGGTKVEIKRTVAAPSVFIFPPSDEQLKSGTASVVC |
| | | LLNNFYPREAKVQWKVDNALQSGNSQESVTEQDSKDSTYSLSST |
| | | LTLSKADYEKHKVYACEVTHQGLSSPVTKSFNRGEC |
| Pidilizumab | | |
| SEQ ID NO: 60 | Heavy | QVQLVQSGSELKKPGASVKISCKASGYTFTNYGMNWVRQAPGQ |
| | chain | GLQWMGWINTDSGESTYAEEFKGRFVFSLDTSVNTAYLQITSLTA |
| | | EDTGMYFCVRVGYDALDYWGQGTLVTVSSASTKGPSVFPLAPSS |
| | | KSTSGGTAALGCLVKDYFPEPVTVSWNSGALTSGVHTFPAVLQSS |
| | | GLYSLSSVVTVPSSSLGTQTYICNVNHKPSNTKVDKRVEPKSCDK |
| | | THTCPPCPAPELLGGPSVFLFPPKPKDTLMISRTPEVTCVVVDVSH |
| | | EDPEVKFNWYVDGVEVHNAKTKPREEQYNSTYRVVSVLTVLHQ |
| | | DWLNGKEYKCKVSNKALPAPIEKTISKAKGQPREPQVYTLPPSRE |
| | | EMTKNQVSLTCLVKGFYPSDIAVEWESNGQPENNYKTTPPVLDS |
| | | DGSFFLYSKLTVDKSRWQQGNVFSCSVMHEALHNHYTQKSLSLS |
| | | PGK |
| SEQ ID NO: 61 | Light | EIVLTQSPSSLSASVGDRVTITCSARSSVSYMHWFQQKPGKAPKL |
| | chain | WIYRTSNLASGVPSRFSGSGSGTSYCLTINSLQPEDFATYYCQQRS |
| | | SFPLTFGGGTKLEIKRTVAAPSVFIFPPSDEQLKSGTASVVCLLNNF |
| | | YPREAKVQWKVDNALQSGNSQESVTEQDSKDSTYSLSSTLTLSK |
| | | ADYEKHKVYACEVTHQGLSSPVTKSFNRGEC |

### PD-L1 Inhibitors

In some embodiments, the compounds of Formula (I) or compounds of Embodiment 16, 17, or 35, or a pharmaceutically acceptable salt, hydrate, solvate, prodrug, stereoisomer, or tautomer thereof, of the present disclosure are used in combination with a PD-L1 inhibitor for treating a disease, e.g., cancer. In some embodiments, the PD-L1 inhibitor is selected from FAZ053 (Novartis), Atezolizumab (Genentech/Roche), Avelumab (Merck Serono and Pfizer), Durvalumab (MedImmune/AstraZeneca), or BMS-936559 (Bristol-Myers Squibb).

### Exemplary PD-L1 Inhibitors

In one embodiment, the PD-L1 inhibitor is an anti-PD-L1 antibody molecule. In one embodiment, the PD-L1 inhibitor is an anti-PD-L1 antibody molecule as disclosed in US 2016/0108123, published on Apr. 21, 2016, entitled “Antibody Molecules to PD-L1 and Uses Thereof,” incorporated by reference in its entirety.

In one embodiment, the anti-PD-L1 antibody molecule comprises at least one, two, three, four, five or six complementarity determining regions (CDRs) (or collectively all of the CDRs) from a heavy and light chain variable region comprising an amino acid sequence shown in Table 5 (e.g., from the heavy and light chain variable region sequences of BAP058-Clone O or BAP058-Clone N disclosed in Table 5), or encoded by a nucleotide sequence shown in Table 5. In some embodiments, the CDRs are according to the Kabat definition (e.g., as set out in Table 5). In some embodiments, the CDRs are according to the Chothia definition (e.g., as set out in Table 5). In some embodiments, the CDRs are according to the combined CDR definitions of both Kabat and Chothia (e.g., as set out in Table 5). In one embodiment, the combination of Kabat and Chothia CDR of VH CDR1 comprises the amino acid sequence GYTFTSYWMY (SEQ ID NO: 214). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 5, or encoded by a nucleotide sequence shown in Table 5.

In one embodiment, the anti-PD-L1 antibody molecule comprises a heavy chain variable region (VH) comprising a VHCDR1 amino acid sequence of SEQ ID NO: 62, a VHCDR2 amino acid sequence of SEQ ID NO: 63, and a VHCDR3 amino acid sequence of SEQ ID NO: 64; and a light chain variable region (VL) comprising a VLCDR1 amino acid sequence of SEQ ID NO: 70, a VLCDR2 amino acid sequence of SEQ ID NO: 71, and a VLCDR3 amino acid sequence of SEQ ID NO: 72, each disclosed in Table 5.

In one embodiment, the anti-PD-L1 antibody molecule comprises a VH comprising a VHCDR1 encoded by the nucleotide sequence of SEQ ID NO: 89, a VHCDR2 encoded by the nucleotide sequence of SEQ ID NO: 90, and a VHCDR3 encoded by the nucleotide sequence of SEQ ID NO: 91; and a VL comprising a VLCDR1 encoded by the nucleotide sequence of SEQ ID NO: 94, a VLCDR2 encoded by the nucleotide sequence of SEQ ID NO: 95, and a VLCDR3 encoded by the nucleotide sequence of SEQ ID NO: 96, each disclosed in Table 5.

In one embodiment, the anti-PD-L1 antibody molecule comprises a VH comprising the amino acid sequence of SEQ ID NO: 67, or an amino acid sequence at least 85%, 90%, 95%, or 99% identical or higher to SEQ ID NO: 67. In one embodiment, the anti-PD-L1 antibody molecule comprises a VL comprising the amino acid sequence of SEQ ID NO: 77, or an amino acid sequence at least 85%, 90%, 95%, or 99% identical or higher to SEQ ID NO: 77. In one embodiment, the anti-PD-L1 antibody molecule comprises a VH comprising the amino acid sequence of SEQ ID NO: 81, or an amino acid sequence at least 85%, 90%, 95%, or 99% identical or higher to SEQ ID NO: 81. In one embodiment, the anti-PD-L1 antibody molecule comprises a VL comprising the amino acid sequence of SEQ ID NO: 85, or an amino acid sequence at least 85%, 90%, 95%, or 99% identical or higher to SEQ ID NO: 85. In one embodiment, the anti-PD-L1 antibody molecule comprises a VH comprising the amino acid sequence of SEQ ID NO: 67 and a VL comprising the amino acid sequence of SEQ ID NO: 77. In one embodiment, the anti-PD-L1 antibody molecule comprises a VH comprising the amino acid sequence of SEQ ID NO: 81 and a VL comprising the amino acid sequence of SEQ ID NO: 85.

In one embodiment, the antibody molecule comprises a VH encoded by the nucleotide sequence of SEQ ID NO: 68, or a nucleotide sequence at least 85%, 90%, 95%, or 99% identical or higher to SEQ ID NO: 68. In one embodiment, the antibody molecule comprises a VL encoded by the nucleotide sequence of SEQ ID NO: 78, or a nucleotide sequence at least 85%, 90%, 95%, or 99% identical or higher to SEQ ID NO: 78. In one embodiment, the antibody molecule comprises a VH encoded by the nucleotide sequence of SEQ ID NO: 82, or a nucleotide sequence at least 85%, 90%, 95%, or 99% identical or higher to SEQ ID NO: 82. In one embodiment, the antibody molecule comprises a VL encoded by the nucleotide sequence of SEQ ID NO: 86, or a nucleotide sequence at least 85%, 90%, 95%, or 99% identical or higher to SEQ ID NO: 86. In one embodiment, the antibody molecule comprises a VH encoded by the nucleotide sequence of SEQ ID NO: 68 and a VL encoded by the nucleotide sequence of SEQ ID NO: 78. In one embodiment, the antibody molecule comprises a VH encoded by the nucleotide sequence of SEQ ID NO: 82 and a VL encoded by the nucleotide sequence of SEQ ID NO: 86.

In one embodiment, the anti-PD-L1 antibody molecule comprises a heavy chain comprising the amino acid sequence of SEQ ID NO: 69, or an amino acid sequence at least 85%, 90%, 95%, or 99% identical or higher to SEQ ID NO: 69. In one embodiment, the anti-PD-L1 antibody molecule comprises a light chain comprising the amino acid sequence of SEQ ID NO: 79, or an amino acid sequence at least 85%, 90%, 95%, or 99% identical or higher to SEQ ID NO: 79. In one embodiment, the anti-PD-L1 antibody molecule comprises a heavy chain comprising the amino acid sequence of SEQ ID NO: 83, or an amino acid sequence at least 85%, 90%, 95%, or 99% identical or higher to SEQ ID NO: 83. In one embodiment, the anti-PD-L1 antibody molecule comprises a light chain comprising the amino acid sequence of SEQ ID NO: 87, or an amino acid sequence at least 85%, 90%, 95%, or 99% identical or higher to SEQ ID NO: 87. In one embodiment, the anti-PD-L1 antibody molecule comprises a heavy chain comprising the amino acid sequence of SEQ ID NO: 69 and a light chain comprising the amino acid sequence of SEQ ID NO: 79. In one embodiment, the anti-PD-L1 antibody molecule comprises a heavy chain comprising the amino acid sequence of SEQ ID NO: 83 and a light chain comprising the amino acid sequence of SEQ ID NO: 87.

In one embodiment, the antibody molecule comprises a heavy chain encoded by the nucleotide sequence of SEQ ID NO: 76, or a nucleotide sequence at least 85%, 90%, 95%, or 99% identical or higher to SEQ ID NO: 76. In one embodiment, the antibody molecule comprises a light chain encoded by the nucleotide sequence of SEQ ID NO: 80, or a nucleotide sequence at least 85%, 90%, 95%, or 99% identical or higher to SEQ ID NO: 80. In one embodiment, the antibody molecule comprises a heavy chain encoded by the nucleotide sequence of SEQ ID NO: 84, or a nucleotide sequence at least 85%, 90%, 95%, or 99% identical or higher to SEQ ID NO: 84. In one embodiment, the antibody molecule comprises a light chain encoded by the nucleotide sequence of SEQ ID NO: 88, or a nucleotide sequence at least 85%, 90%, 95%, or 99% identical or higher to SEQ ID NO: 88. In one embodiment, the antibody molecule comprises a heavy chain encoded by the nucleotide sequence of SEQ ID NO: 76 and a light chain encoded by the nucleotide sequence of SEQ ID NO: 80. In one embodiment, the antibody molecule comprises a heavy chain encoded by the nucleotide sequence of SEQ ID NO: 84 and a light chain encoded by the nucleotide sequence of SEQ ID NO: 88.

The antibody molecules described herein can be made by vectors, host cells, and methods described in US 2016/0108123, incorporated by reference in its entirety.

TABLE 5
Amino acid and nucleotide sequences of exemplary
anti-PD-Ll antibody molecules

| | | |
|---|---|---|
| BAP058-Clone O HC | | |
| SEQ ID NO: 62 | HCDR1 | SYWMY |
| (Kabat) | | |
| SEQ ID NO: 63 | HCDR2 | RIDPNSGSTKYNEKFKN |
| (Kabat) | | |
| SEQ ID NO: 64 | HCDR3 | DYRKGLYAMDY |
| (Kabat) | | |
| SEQ ID NO: 65 | HCDR1 | GYTFTSY |
| (Chothia) | | |
| SEQ ID NO: 66 | HCDR2 | DPNSGS |
| (Chothia) | | |
| SEQ ID NO: 64 | HCDR3 | DYRKGLYAMDY |
| (Chothia) | | |
| SEQ ID NO: 67 | VH | EVQLVQSGAEVKKPGATVKISCKVSGYTFTSYWMYWVR |
| | | QARGQRLEWIGRIDPNSGSTKYNEKFKNRFTISRDNSKNTL |
| | | YLQMNSLRAEDTAVYYCARDYRKGLYAMDYWGQGTTV |
| | | TVSS |
| SEQ ID NO: 68 | DNA VH | GAAGTGCAGCTGGTGCAGTCAGGCGCCGAAGTGAAGAA |
| | | ACCCGGCGCTACCGTGAAGATTAGCTGTAAAGTCTCAG |
| | | GCTACACCTTCACTAGCTACTGGATGTACTGGGTCCGAC |
| | | AGGCTAGAGGGCAAAGACTGGAGTGGATCGGTAGAATC |
| | | GACCCTAATAGCGGCTCTACTAAGTATAACGAGAAGTT |
| | | TAAGAATAGGTTCACTATTAGTAGGGATAACTCTAAGA |
| | | ACACCCTGTACCTGCAGATGAATAGCCTGAGAGCCGAG |
| | | GACACCGCCGTCTACTACTGCGCTAGAGACTATAGAAA |
| | | GGGCCTGTACGCTATGGACTACTGGGGTCAAGGCACTA |
| | | CCGTGACCGTGTCTTCA |
| SEQ ID NO: 69 | Heavy | EVQLVQSGAEVKKPGATVKISCKVSGYTFTSYWMYWVR |
| | chain | QARGQRLEWIGRIDPNSGSTKYNEKFKNRFTISRDNSKNTL |
| | | YLQMNSLRAEDTAVYYCARDYRKGLYAMDYWGQGTTV |
| | | TVSSASTKGPSVFPLAPCSRSTSESTAALGCLVKDYFPEPV |
| | | TVSWNSGALTSGVHTFPAVLQSSGLYSLSSVVTVPSSSLGT |
| | | KTYTCNVDHKPSNTKVDKRVESKYGPPCPPCPAPEFLGGP |
| | | SVFLFPPKPKDTLMISRTPEVTCVVVDVSQEDPEVQFNWY |
| | | VDGVEVHNAKTKPREEQFNSTYRVVSVLTVLHQDWLNG |
| | | KEYKCKVSNKGLPSSIEKTISKAKGQPREPQVYTLPPSQEE |
| | | MTKNQVSLTCLVKGFYPSDIAVEWESNGQPENNYKTTPPV |
| | | LDSDGSFFLYSRLTVDKSRWQEGNVFSCSVMHEALHNHY |
| | | TQKSLSLSLG |
| SEQ ID NO: 76 | DNA | GAAGTGCAGCTGGTGCAGTCAGGCGCCGAAGTGAAGAA |
| | heavy | ACCCGGCGCTACCGTGAAGATTAGCTGTAAAGTCTCAG |
| | chain | GCTACACCTTCACTAGCTACTGGATGTACTGGGTCCGAC |
| | | AGGCTAGAGGGCAAAGACTGGAGTGGATCGGTAGAATC |
| | | GACCCTAATAGCGGCTCTACTAAGTATAACGAGAAGTT |
| | | TAAGAATAGGTTCACTATTAGTAGGGATAACTCTAAGA |
| | | ACACCCTGTACCTGCAGATGAATAGCCTGAGAGCCGAG |
| | | GACACCGCCGTCTACTACTGCGCTAGAGACTATAGAAA |
| | | GGGCCTGTACGCTATGGACTACTGGGGTCAAGGCACTA |
| | | CCGTGACCGTGTCTTCAGCTAGCACTAAGGGCCCGTCCG |
| | | TGTTCCCCCTGGCACCTTGTAGCCGGAGCACTAGCGAAT |
| | | CCACCGCTGCCCTCGGCTGCCTGGTCAAGGATTACTTCC |
| | | CGGAGCCCGTGACCGTGTCCTGGAACAGCGGAGCCCTG |
| | | ACCTCCGGAGTGCACACCTTCCCCGCTGTGCTGCAGAGC |
| | | TCCGGGCTGTACTCGCTGTCGTCGGTGGTCACGGTGCCT |
| | | TCATCTAGCCTGGGTACCAAGACCTACACTTGCAACGTG |
| | | GACCACAAGCCTTCCAACACTAAGGTGGACAAGCGCGT |
| | | CGAATCGAAGTACGGCCCACCGTGCCCGCCTTGTCCCG |
| | | CGCCGGAGTTCCTCGGCGGTCCCTCGGTCTTTCTGTTCC |
| | | CACCGAAGCCCAAGGACACTTTGATGATTTCCCGCACC |
| | | CCTGAAGTGACATGCGTGGTCGTGGACGTGTCACAGGA |
| | | AGATCCGGAGGTGCAGTTCAATTGGTACGTGGATGGCG |
| | | TCGAGGTGCACAACGCCAAAACCAAGCCGAGGGAGGA |
| | | GCAGTTCAACTCCACTTACCGCGTCGTGTCCGTGCTGAC |
| | | GGTGCTGCATCAGGACTGGCTGAACGGGAAGGAGTACA |
| | | AGTGCAAAGTGTCCAACAAGGGACTTCCTAGCTCAATC |
| | | GAAAAGACCATCTCGAAAGCCAAGGGACAGCCCCGGG |
| | | AACCCCAAGTGTATACCCTGCCACCGAGCCAGGAAGAA |
| | | ATGACTAAGAACCAAGTCTCATTGACTTGCCTTGTGAAG |
| | | GGCTTCTACCCATCGGATATCGCCGTGGAATGGGAGTC |
| | | CAACGGCCAGCCGGAAAACAACTACAAGACCACCCCTC |
| | | CGGTGCTGGACTCAGACGGATCCTTCTTCCTCTACTCGC |
| | | GGCTGACCGTGGATAAGAGCAGATGGCAGGAGGGAAA |
| | | TGTGTTCAGCTGTTCTGTGATGCATGAAGCCCTGCACAA |
| | | CCACTACACTCAGAAGTCCCTGTCCCTCTCCCTGGGA |
| BAP058-Clone O LC | | |
| SEQ ID NO: 70 | LCDR1 | KASQDVGTAVA |
| (Kabat) | | |
| SEQ ID NO: 71 | LCDR2 | WASTRHT |
| (Kabat) | | |
| SEQ ID NO: 72 | LCDR3 | QQYNSYPLT |
| (Kabat) | | |
| SEQ ID NO: 73 | LCDR1 | SQDVGTA |
| (Chothia) | | |
| SEQ ID NO: 74 | LCDR2 | WAS |
| (Chothia) | | |
| SEQ ID NO: 75 | LCDR3 | YNSYPL |
| (Chothia) | | |
| SEQ ID NO: 77 | VL | AIQLTQSPSSLSASVGDRVTITCKASQDVGTAVAWYLQKP |
| | | GQSPQLLIYWASTRHTGVPSRFSGSGSGTDFTFTISSLEAED |
| | | AATYYCQQYNSYPLTFGQGTKVEIK |
| SEQ ID NO: 78 | DNA VL | GCTATTCAGCTGACTCAGTCACCTAGTAGCCTGAGCGCT |
| | | AGTGTGGGCGATAGAGTGACTATCACCTGTAAAGCCTC |
| | | TCAGGACGTGGGCACCGCCGTGGCCTGGTATCTGCAGA |
| | | AGCCTGGTCAATCACCTCAGCTGCTGATCTACTGGGCCT |
| | | CTACTAGACACACCGGCGTGCCCTCTAGGTTTAGCGGTA |
| | | GCGGTAGTGGCACCGACTTCACCTTCACTATCTCTTCAC |
| | | TGGAAGCCGAGGACGCCGCTACCTACTACTGTCAGCAG |
| | | TATAATAGCTACCCCCTGACCTTCGGTCAAGGCACTAAG |
| | | GTCGAGATTAAG |
| SEQ ID NO: 79 | Light | AIQLTQSPSSLSASVGDRVTITCKASQDVGTAVAWYLQKP |
| | chain | GQSPQLLIYWASTRHTGVPSRFSGSGSGTDFTFTISSLEAED |
| | | AATYYCQQYNSYPLTFGQGTKVEIKRTVAAPSVFIFPPSDE |
| | | QLKSGTASVVCLLNNFYPREAKVQWKVDNALQSGNSQES |
| | | VTEQDSKDSTYSLSSTLTLSKADYEKHKVYACEVTHQGLS |
| | | SPVTKSFNRGEC |
| SEQ ID NO: 80 | DNA | GCTATTCAGCTGACTCAGTCACCTAGTAGCCTGAGCGCT |
| | light | AGTGTGGGCGATAGAGTGACTATCACCTGTAAAGCCTC |
| | chain | TCAGGACGTGGGCACCGCCGTGGCCTGGTATCTGCAGA |
| | | AGCCTGGTCAATCACCTCAGCTGCTGATCTACTGGGCCT |
| | | CTACTAGACACACCGGCGTGCCCTCTAGGTTTAGCGGTA |
| | | GCGGTAGTGGCACCGACTTCACCTTCACTATCTCTTCAC |
| | | TGGAAGCCGAGGACGCCGCTACCTACTACTGTCAGCAG |
| | | TATAATAGCTACCCCCTGACCTTCGGTCAAGGCACTAAG |
| | | GTCGAGATTAAGCGTACGGTGGCCGCTCCCAGCGTGTT |
| | | CATCTTCCCCCCCAGCGACGAGCAGCTGAAGAGCGGCA |
| | | CCGCCAGCGTGGTGTGCCTGCTGAACAACTTCTACCCCC |
| | | GGGAGGCCAAGGTGCAGTGGAAGGTGGACAACGCCCT |
| | | GCAGAGCGGCAACAGCCAGGAGAGCGTCACCGAGCAG |
| | | GACAGCAAGGACTCCACCTACAGCCTGAGCAGCACCCT |
| | | GACCCTGAGCAAGGCCGACTACGAGAAGCATAAGGTGT |
| | | ACGCCTGCGAGGTGACCCACCAGGGCCTGTCCAGCCCC |
| | | GTGACCAAGAGCTTCAACAGGGGCGAGTGC |
| BAP058-Clone N HC | | |
| SEQ ID NO: 62 | HCDR1 | SYWMY |
| (Kabat) | | |
| SEQ ID NO: 63 | HCDR2 | RIDPNSGSTKYNEKFKN |
| (Kabat) | | |
| SEQ ID NO: 64 | HCDR3 | DYRKGLYAMDY |
| (Kabat) | | |
| SEQ ID NO: 65 | HCDR1 | GYTFTSY |
| (Chothia) | | |
| SEQ ID NO: 66 | HCDR2 | DPNSGS |
| (Chothia) | | |
| SEQ ID NO: 64 | HCDR3 | DYRKGLYAMDY |
| (Chothia) | | |
| SEQ ID NO: 81 | VH | EVQLVQSGAEVKKPGATVKISCKVSGYTFTSYWMYWVR |
| | | QATGQGLEWMGRIDPNSGSTKYNEKFKNRVTITADKSTST |
| | | AYMELSSLRSEDTAVYYCARDYRKGLYAMDYWGQGTTV |
| | | TVSS |
| SEQ ID NO: 82 | DNA VH | GAAGTGCAGCTGGTGCAGTCAGGCGCCGAAGTGAAGAA |
| | | ACCCGGCGCTACCGTGAAGATTAGCTGTAAAGTCTCAG |
| | | GCTACACCTTCACTAGCTACTGGATGTACTGGGTCCGAC |
| | | AGGCTACCGGTCAAGGCCTGGAGTGGATGGGTAGAATC |
| | | GACCCTAATAGCGGCTCTACTAAGTATAACGAGAAGTT |
| | | TAAGAATAGAGTGACTATCACCGCCGATAAGTCTACTA |
| | | GCACCGCCTATATGGAACTGTCTAGCCTGAGATCAGAG |
| | | GACACCGCCGTCTACTACTGCGCTAGAGACTATAGAAA |
| | | GGGCCTGTACGCTATGGACTACTGGGGTCAAGGCACTA |
| | | CCGTGACCGTGTCTTCA |
| SEQ ID NO: 83 | Heavy | EVQLVQSGAEVKKPGATVKISCKVSGYTFTSYWMYWVR |
| | chain | QATGQGLEWMGRIDPNSGSTKYNEKFKNRVTITADKSTST |
| | | AYMELSSLRSEDTAVYYCARDYRKGLYAMDYWGQGTTV |
| | | TVSSASTKGPSVFPLAPCSRSTSESTAALGCLVKDYFPEPV |
| | | TVSWNSGALTSGVHTFPAVLQSSGLYSLSSVVTVPSSSLGT |
| | | KTYTCNVDHKPSNTKVDKRVESKYGPPCPPCPAPEFLGGP |
| | | SVFLFPPKPKDTLMISRTPEVTCVVVDVSQEDPEVQFNWY |
| | | VDGVEVHNAKTKPREEQFNSTYRVVSVLTVLHQDWLNG |
| | | KEYKCKVSNKGLPSSIEKTISKAKGQPREPQVYTLPPSQEE |
| | | MTKNQVSLTCLVKGFYPSDIAVEWESNGQPENNYKTTPPV |
| | | LDSDGSFFLYSRLTVDKSRWQEGNVFSCSVMHEALHNHY |
| | | TQKSLSLSLG |
| SEQ ID NO: 84 | DNA | GAAGTGCAGCTGGTGCAGTCAGGCGCCGAAGTGAAGAA |
| | heavy | ACCCGGCGCTACCGTGAAGATTAGCTGTAAAGTCTCAG |
| | chain | GCTACACCTTCACTAGCTACTGGATGTACTGGGTCCGAC |
| | | AGGCTACCGGTCAAGGCCTGGAGTGGATGGGTAGAATC |
| | | GACCCTAATAGCGGCTCTACTAAGTATAACGAGAAGTT |
| | | TAAGAATAGAGTGACTATCACCGCCGATAAGTCTACTA |
| | | GCACCGCCTATATGGAACTGTCTAGCCTGAGATCAGAG |
| | | GACACCGCCGTCTACTACTGCGCTAGAGACTATAGAAA |
| | | GGGCCTGTACGCTATGGACTACTGGGGTCAAGGCACTA |
| | | CCGTGACCGTGTCTTCAGCTAGCACTAAGGGCCCGTCCG |
| | | TGTTCCCCCTGGCACCTTGTAGCCGGAGCACTAGCGAAT |
| | | CCACCGCTGCCCTCGGCTGCCTGGTCAAGGATTACTTCC |
| | | CGGAGCCCGTGACCGTGTCCTGGAACAGCGGAGCCCTG |
| | | ACCTCCGGAGTGCACACCTTCCCCGCTGTGCTGCAGAGC |
| | | TCCGGGCTGTACTCGCTGTCGTCGGTGGTCACGGTGCCT |
| | | TCATCTAGCCTGGGTACCAAGACCTACACTTGCAACGTG |
| | | GACCACAAGCCTTCCAACACTAAGGTGGACAAGCGCGT |
| | | CGAATCGAAGTACGGCCCACCGTGCCCGCCTTGTCCCG |
| | | CGCCGGAGTTCCTCGGCGGTCCCTCGGTCTTTCTGTTCC |
| | | CACCGAAGCCCAAGGACACTTTGATGATTTCCCGCACC |
| | | CCTGAAGTGACATGCGTGGTCGTGGACGTGTCACAGGA |
| | | AGATCCGGAGGTGCAGTTCAATTGGTACGTGGATGGCG |
| | | TCGAGGTGCACAACGCCAAAACCAAGCCGAGGGAGGA |
| | | GCAGTTCAACTCCACTTACCGCGTCGTGTCCGTGCTGAC |
| | | GGTGCTGCATCAGGACTGGCTGAACGGGAAGGAGTACA |
| | | AGTGCAAAGTGTCCAACAAGGGACTTCCTAGCTCAATC |
| | | GAAAAGACCATCTCGAAAGCCAAGGGACAGCCCCGGG |
| | | AACCCCAAGTGTATACCCTGCCACCGAGCCAGGAAGAA |
| | | ATGACTAAGAACCAAGTCTCATTGACTTGCCTTGTGAAG |
| | | GGCTTCTACCCATCGGATATCGCCGTGGAATGGGAGTC |
| | | CAACGGCCAGCCGGAAAACAACTACAAGACCACCCCTC |
| | | CGGTGCTGGACTCAGACGGATCCTTCTTCCTCTACTCGC |
| | | GGCTGACCGTGGATAAGAGCAGATGGCAGGAGGGAAA |
| | | TGTGTTCAGCTGTTCTGTGATGCATGAAGCCCTGCACAA |
| | | CCACTACACTCAGAAGTCCCTGTCCCTCTCCCTGGGA |
| BAP058-Clone N LC | | |
| SEQ ID NO: 70 | LCDR1 | KASQDVGTAVA |
| (Kabat) | | |
| SEQ ID NO: 71 | LCDR2 | WASTRHT |
| (Kabat) | | |
| SEQ ID NO: 72 | LCDR3 | QQYNSYPLT |
| (Kabat) | | |
| SEQ ID NO: 73 | LCDR1 | SQDVGTA |
| (Chothia) | | |
| SEQ ID NO: 74 | LCDR2 | WAS |
| (Chothia) | | |
| SEQ ID NO: 75 | LCDR3 | YNSYPL |
| (Chothia) | | |
| SEQ ID NO: 85 | VL | DVVMTQSPLSLPVTLGQPASISCKASQDVGTAVAWYQQK |
| | | PGQAPRLLIYWASTRHTGVPSRFSGSGSGTEFTLTISSLQPD |
| | | DFATYYCQQYNSYPLTFGQGTKVEIK |
| SEQ ID NO: 86 | DNA VL | GACGTCGTGATGACTCAGTCACCCCTGAGCCTGCCCGTG |
| | | ACCCTGGGGCAGCCCGCCTCTATTAGCTGTAAAGCCTCT |
| | | CAGGACGTGGGCACCGCCGTGGCCTGGTATCAGCAGAA |
| | | GCCAGGGCAAGCCCCTAGACTGCTGATCTACTGGGCCT |
| | | CTACTAGACACACCGGCGTGCCCTCTAGGTTTAGCGGTA |
| | | GCGGTAGTGGCACCGAGTTCACCCTGACTATCTCTTCAC |
| | | TGCAGCCCGACGACTTCGCTACCTACTACTGTCAGCAGT |
| | | ATAATAGCTACCCCCTGACCTTCGGTCAAGGCACTAAG |
| | | GTCGAGATTAAG |
| SEQ ID NO: 87 | Light | DVVMTQSPLSLPVTLGQPASISCKASQDVGTAVAWYQQK |
| | chain | PGQAPRLLIYWASTRHTGVPSRFSGSGSGTEFTLTISSLQPD |
| | | DFATYYCQQYNSYPLTFGQGTKVEIKRTVAAPSVFIFPPSD |
| | | EQLKSGTASVVCLLNNFYPREAKVQWKVDNALQSGNSQE |
| | | SVTEQDSKDSTYSLSSTLTLSKADYEKHKVYACEVTHQGL |
| | | SSPVTKSFNRGEC |
| SEQ ID NO: 88 | DNA | GACGTCGTGATGACTCAGTCACCCCTGAGCCTGCCCGTG |
| | light | ACCCTGGGGCAGCCCGCCTCTATTAGCTGTAAAGCCTCT |
| | chain | CAGGACGTGGGCACCGCCGTGGCCTGGTATCAGCAGAA |
| | | GCCAGGGCAAGCCCCTAGACTGCTGATCTACTGGGCCT |
| | | CTACTAGACACACCGGCGTGCCCTCTAGGTTTAGCGGTA |
| | | GCGGTAGTGGCACCGAGTTCACCCTGACTATCTCTTCAC |
| | | TGCAGCCCGACGACTTCGCTACCTACTACTGTCAGCAGT |
| | | ATAATAGCTACCCCCTGACCTTCGGTCAAGGCACTAAG |
| | | GTCGAGATTAAGCGTACGGTGGCCGCTCCCAGCGTGTT |
| | | CATCTTCCCCCCCAGCGACGAGCAGCTGAAGAGCGGCA |
| | | CCGCCAGCGTGGTGTGCCTGCTGAACAACTTCTACCCCC |
| | | GGGAGGCCAAGGTGCAGTGGAAGGTGGACAACGCCCT |
| | | GCAGAGCGGCAACAGCCAGGAGAGCGTCACCGAGCAG |
| | | GACAGCAAGGACTCCACCTACAGCCTGAGCAGCACCCT |
| | | GACCCTGAGCAAGGCCGACTACGAGAAGCATAAGGTGT |
| | | ACGCCTGCGAGGTGACCCACCAGGGCCTGTCCAGCCCC |
| | | GTGACCAAGAGCTTCAACAGGGGCGAGTGC |
| BAP058-Clone O HC | | |
| SEQ ID NO: 89 | HCDR1 | agctactggatgtac |
| (Kabat) | | |
| SEQ ID NO: 90 | HCDR2 | agaatcgaccctaatagcggctctactaagtataacgagaagtttaagaat |
| (Kabat) | | |
| SEQ ID NO: 91 | HCDR3 | gactatagaaagggcctgtacgctatggactac |
| (Kabat) | | |
| SEQ ID NO: 92 | HCDR1 | ggctacaccttcactagctac |
| (Chothia) | | |
| SEQ ID NO: 93 | HCDR2 | gaccctaatagcggctct |
| (Chothia) | | |
| SEQ ID NO: 91 | HCDR3 | gactatagaaagggcctgtacgctatggactac |
| (Chothia) | | |
| BAP058-Clone O LC | | |
| SEQ ID NO: 94 | LCDR1 | aaagcctctcaggacgtgggcaccgccgtggcc |
| (Kabat) | | |
| SEQ ID NO: 95 | LCDR2 | tgggcctctactagacacacc |
| (Kabat) | | |
| SEQ ID NO: 96 | LCDR3 | cagcagtataatagctaccccctgacc |
| (Kabat) | | |
| SEQ ID NO: 97 | LCDR1 | tctcaggacgtgggcaccgcc |
| (Chothia) | | |
| SEQ ID NO: 98 | LCDR2 | tgggcctct |
| (Chothia) | | |
| SEQ ID NO: 99 | LCDR3 | tataatagctaccccctg |
| (Chothia) | | |
| BAP058-Clone N HC | | |
| SEQ ID NO: 89 | HCDR1 | agctactggatgtac |
| (Kabat) | | |
| SEQ ID NO: 90 | HCDR2 | agaatcgaccctaatagcggctctactaagtataacgagaagtttaagaat |
| (Kabat) | | |
| SEQ ID NO: 91 | HCDR3 | gactatagaaagggcctgtacgctatggactac |
| (Kabat) | | |
| SEQ ID NO: 92 | HCDR1 | ggctacaccttcactagctac |
| (Chothia) | | |
| SEQ ID NO: 93 | HCDR2 | gaccctaatagcggctct |
| (Chothia) | | |
| SEQ ID NO: 91 | HCDR3 | gactatagaaagggcctgtacgctatggactac |
| (Chothia) | | |
| BAP058-Clone N LC | | |
| SEQ ID NO: 94 | LCDR1 | aaagcctctcaggacgtgggcaccgccgtggcc |
| (Kabat) | | |
| SEQ ID NO: 95 | LCDR2 | tgggcctctactagacacacc |
| (Kabat) | | |
| SEQ ID NO: 96 | LCDR3 | cagcagtataatagctaccccctgacc |
| (Kabat) | | |
| SEQ ID NO: 97 | LCDR1 | tctcaggacgtgggcaccgcc |
| (Chothia) | | |
| SEQ ID NO: 98 | LCDR2 | tgggcctct |
| (Chothia) | | |
| SEQ ID NO: 99 | LCDR3 | tataatagctaccccctg |
| (Chothia) | | |

### Other Exemplary PD-L1 Inhibitors

In some embodiments, the PD-L1 inhibitor is anti-PD-L1 antibody. In some embodiments, the anti-PD-L1 inhibitor is selected from YW243.55.570, MPDL3280A, MEDI-4736, or MDX-1105MSB-0010718C (also referred to as A09-246-2) disclosed in, e.g., WO 2013/0179174, and having a sequence disclosed herein (or a sequence substantially identical or similar thereto, e.g., a sequence at least 85%, 90%, 95% identical or higher to the sequence specified).

In one embodiment, the PD-L1 inhibitor is MDX-1105. MDX-1105, also known as BMS-936559, is an anti-PD-L1 antibody described in PCT Publication No. WO 2007/005874.

In one embodiment, the PD-L1 inhibitor is YW243.55.570. The YW243.55.570 antibody is an anti-PD-L1 described in PCT Publication No. WO 2010/077634.

In one embodiment, the PD-L1 inhibitor is MDPL3280A (Genentech/Roche) also known as Atezolizumabm, RG7446, RO5541267, YW243.55. 570, or TECENTRIQ™. MDPL3280A is a human Fc optimized IgG1 monoclonal antibody that binds to PD-L1. MDPL3280A and other human monoclonal antibodies to PD-L1 are disclosed in U.S. Pat. No. 7,943,743 and U.S Publication No.: 20120039906 incorporated by reference in its entirety. In one embodiment, the anti-PD-L1 antibody molecule comprises one or more of the CDR sequences (or collectively all of the CDR sequences), the heavy chain or light chain variable region sequence, or the heavy chain or light chain sequence of Atezolizumab, e.g., as disclosed in Table 6.

In other embodiments, the PD-L2 inhibitor is AMP-224. AMP-224 is a PD-L2 Fc fusion soluble receptor that blocks the interaction between PD1 and B7-H1 (B7-DCIg; Amplimmune; e.g., disclosed in PCT Publication Nos. WO2010/027827 and WO2011/066342).

In one embodiment, the PD-L1 inhibitor is an anti-PD-L1 antibody molecule. In one embodiment, the anti-PD-L1 antibody molecule is Avelumab (Merck Serono and Pfizer), also known as MSB0010718C. Avelumab and other anti-PD-L1 antibodies are disclosed in WO 2013/079174, incorporated by reference in its entirety. In one embodiment, the anti-PD-L1 antibody molecule comprises one or more of the CDR sequences (or collectively all of the CDR sequences), the heavy chain or light chain variable region sequence, or the heavy chain or light chain sequence of Avelumab, e.g., as disclosed in Table 6.

In one embodiment, the anti-PD-L1 antibody molecule is Durvalumab (MedImmune/AstraZeneca), also known as MEDI4736. Durvalumab and other anti-PD-L1 antibodies are disclosed in U.S. Pat. No. 8,779,108, incorporated by reference in its entirety. In one embodiment, the anti-PD-L1 antibody molecule comprises one or more of the CDR sequences (or collectively all of the CDR sequences), the heavy chain or light chain variable region sequence, or the heavy chain or light chain sequence of Durvalumab, e.g., as disclosed in Table 6.

In one embodiment, the anti-PD-L1 antibody molecule is BMS-936559 (Bristol-Myers Squibb), also known as MDX-1105 or 12A4. BMS-936559 and other anti-PD-L1 antibodies are disclosed in U.S. Pat. No. 7,943,743 and WO 2015/081158, incorporated by reference in their entirety. In one embodiment, the anti-PD-L1 antibody molecule comprises one or more of the CDR sequences (or collectively all of the CDR sequences), the heavy chain or light chain variable region sequence, or the heavy chain or light chain sequence of BMS-936559, e.g., as disclosed in Table 6.

Further known anti-PD-L1 antibodies include those described, e.g., in WO 2015/181342, WO 2014/100079, WO 2016/000619, WO 2014/022758, WO 2014/055897, WO 2015/061668, WO 2013/079174, WO 2012/145493, WO 2015/112805, WO 2015/109124, WO 2015/195163, U.S. Pat. Nos. 8,168,179, 8,552,154, 8,460,927, and 9,175,082, incorporated by reference in their entirety.

In one embodiment, the anti-PD-L1 antibody is an antibody that competes for binding with, and/or binds to the same epitope on PD-L1 as, one of the anti-PD-L1 antibodies described herein.

TABLE 6
Amino acid sequences of other
exemplary anti-PD-L1 antibody
molecules

| | | |
|---|---|---|
| Atezolizumab | | |
| SEQ ID NO: | Heavy | EVQLVESGGGLVQPGGSLRLSCAASGFTFSD |
| 100 | chain | SWIHWVRQAPGKGLEWVAWISPYGGSTYYAD |
| | | SVKGRFTISADTSKNTAYLQMNSLRAEDTAV |
| | | YYCARRHWPGGFDYWGQGTLVTVSSASTKGP |
| | | SVFPLAPSSKSTSGGTAALGCLVKDYFPEPV |
| | | TVSWNSGALTSGVHTFPAVLQSSGLYSLSSV |
| | | VTVPSSSLGTQTYICNVNHKPSNTKVDKKVE |
| | | PKSCDKTHTCPPCPAPELLGGPSVFLFPPKP |
| | | KDTLMISRTPEVTCVVVDVSHEDPEVKFNWY |
| | | VDGVEVHNAKTKPREEQYASTYRVVSVLTVL |
| | | HQDWLNGKEYKCKVSNKALPAPIEKTISKAK |
| | | GQPREPQVYTLPPSREEMTKNQVSLTCLVKG |
| | | FYPSDIAVEWESNGQPENNYKTTPPVLDSDG |
| | | SFFLYSKLTVDKSRWQQGNVFSCSVMHEALH |
| | | NHYTQKSLSLSPGK |
| SEQ ID NO: | Light | DIQMTQSPSSLSASVGDRVTITCRASQDVST |
| 101 | chain | AVAWYQQKPGKAPKLLIYSASFLYSGVPSRF |
| | | SGSGSGTDFTLTISSLQPEDFATYYCQQYLY |
| | | HPATFGQGTKVEIKRTVAAPSVFIFPPSDEQ |
| | | LKSGTASVVCLLNNFYPREAKVQWKVDNALQ |
| | | SGNSQESVTEQDSKDSTYSLSSTLTLSKADY |
| | | EKHKVYACEVTHQGLSSPVTKSFNRGEC |
| Avelumab | | |
| SEQ ID NO: | Heavy | EVQLLESGGGLVQPGGSLRLSCAASGFTFSS |
| 102 | chain | YIMMWVRQAPGKGLEWVSSIYPSGGITFYAD |
| | | TVKGRFTISRDNSKNTLYLQMNSLRAEDTAV |
| | | YYCARIKLGTVTTVDYWGQGTLVTVSSASTK |
| | | GPSVFPLAPSSKSTSGGTAALGCLVKDYFPE |
| | | PVTVSWNSGALTSGVHTFPAVLQSSGLYSLS |
| | | SVVTVPSSSLGTQTYICNVNHKPSNTKVDKK |
| | | VEPKSCDKTHTCPPCPAPELLGGPSVFLFPP |
| | | KPKDTLMISRTPEVTCVVVDVSHEDPEVKFN |
| | | WYVDGVEVHNAKTKPREEQYNSTYRVVSVLT |
| | | VLHQDWLNGKEYKCKVSNKALPAPIEKTISK |
| | | AKGQPREPQVYTLPPSRDELTKNQVSLTCLV |
| | | KGFYPSDIAVEWESNGQPENNYKTTPPVLDS |
| | | DGSFFLYSKLTVDKSRWQQGNVFSCSVMHEA |
| | | LHNHYTQKSLSLSPGK |
| SEQ ID NO: | Light | QSALTQPASVSGSPGQSITISCTGTSSDVGG |
| 103 | chain | YNYVSWYQQHPGKAPKLMIYDVSNRPSGVSN |
| | | RFSGSKSGNTASLTISGLQAEDEADYYCSSY |
| | | TSSSTRVFGTGTKVTVLGQPKANPTVTLFPP |
| | | SSEELQANKATLVCLISDFYPGAVTVAWKAD |
| | | GSPVKAGVETTKPSKQSNNKYAASSYLSLTP |
| | | EQWKSHRSYSCQVTHEGSTVEKTVAPTECS |
| Durvalumab | | |
| SEQ ID NO: | Heavy | EVQLVESGGGLVQPGGSLRLSCAASGFTFSR |
| 104 | chain | YWMSWVRQAPGKGLEWVANIKQDGSEKYYVD |
| | | SVKGRFTISRDNAKNSLYLQMNSLRAEDTAV |
| | | YYCAREGGWFGELAFDYWGQGTLVTVSSAST |
| | | KGPSVFPLAPSSKSTSGGTAALGCLVKDYFP |
| | | EPVTVSWNSGALTSGVHTFPAVLQSSGLYSL |
| | | SSVVTVPSSSLGTQTYICNVNHKPSNTKVDK |
| | | RVEPKSCDKTHTCPPCPAPEFEGGPSVFLFP |
| | | PKPKDTLMISRTPEVTCVVVDVSHEDPEVKF |
| | | NWYVDGVEVHNAKTKPREEQYNSTYRVVSVL |
| | | TVLHQDWLNGKEYKCKVSNKALPASIEKTIS |
| | | KAKGQPREPQVYTLPPSREEMTKNQVSLTCL |
| | | VKGFYPSDIAVEWESNGQPENNYKTTPPVLD |
| | | SDGSFFLYSKLTVDKSRWQQGNVFSCSVMHE |
| | | ALHNHYTQKSLSLSPGK |
| SEQ ID NO: | Light | EIVLTQSPGTLSLSPGERATLSCRASQRVSS |
| 105 | chain | SYLAWYQQKPGQAPRLLIYDASSRATGIPDR |
| | | FSGSGSGTDFTLTISRLEPEDFAVYYCQQYG |
| | | SLPWTFGQGTKVEIKRTVAAPSVFIFPPSDE |
| | | QLKSGTASVVCLLNNFYPREAKVQWKVDNAL |
| | | QSGNSQESVTEQDSKDSTYSLSSTLTLSKAD |
| | | YEKHKVYACEVTHQGLSSPVTKSFNRGEC |
| BMS-936559 | | |
| SEQ ID NO: | VH | QVQLVQSGAEVKKPGSSVKVSCKTSGDTFST |
| 106 | | YAISWVRQAPGQGLEWMGGIIPIFGKAHYAQ |
| | | KFQGRVTITADESTSTAYMELSSLRSEDTAV |
| | | YFCARKFHFVSGSPFGMDVWGQGTTVTVSS |
| SEQ ID NO: | VL | EIVLTQSPATLSLSPGERATLSCRASQSVSS |
| 107 | | YLAWYQQKPGQAPRLLIYDASNRATGIPARF |
| | | SGSGSGTDFTLTISSLEPEDFAVYYCQQRSN |
| | | WPTFGQGTKVEIK |

### LAG-3 Inhibitors

In some embodiments, the compounds of Formula (I) or compounds of Embodiment 16, 17, or 35, or a pharmaceutically acceptable salt, hydrate, solvate, prodrug, stereoisomer, or tautomer thereof, of the present disclosure are used in combination with a LAG-3 inhibitor to treat a disease, e.g., cancer. In some embodiments, the LAG-3 inhibitor is selected from LAG525 (Novartis), BMS-986016 (Bristol-Myers Squibb), or TSR-033 (Tesaro).

### Exemplary LAG-3 Inhibitors

In one embodiment, the LAG-3 inhibitor is an anti-LAG-3 antibody molecule. In one embodiment, the LAG-3 inhibitor is an anti-LAG-3 antibody molecule as disclosed in US 2015/0259420, published on Sep. 17, 2015, entitled “Antibody Molecules to LAG-3 and Uses Thereof,” incorporated by reference in its entirety.

In one embodiment, the anti-LAG-3 antibody molecule comprises at least one, two, three, four, five or six complementarity determining regions (CDRs) (or collectively all of the CDRs) from a heavy and light chain variable region comprising an amino acid sequence shown in Table 7 (e.g., from the heavy and light chain variable region sequences of BAP050-Clone I or BAP050-Clone J disclosed in Table 7), or encoded by a nucleotide sequence shown in Table 7. In some embodiments, the CDRs are according to the Kabat definition (e.g., as set out in Table 7). In some embodiments, the CDRs are according to the Chothia definition (e.g., as set out in Table 7). In some embodiments, the CDRs are according to the combined CDR definitions of both Kabat and Chothia (e.g., as set out in Table 7). In one embodiment, the combination of Kabat and Chothia CDR of VH CDR1 comprises the amino acid sequence GFTLTNYGMN (SEQ ID NO: 173). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 7, or encoded by a nucleotide sequence shown in Table 7.

In one embodiment, the anti-LAG-3 antibody molecule comprises a heavy chain variable region (VH) comprising a VHCDR1 amino acid sequence of SEQ ID NO: 108, a VHCDR2 amino acid sequence of SEQ ID NO: 109, and a VHCDR3 amino acid sequence of SEQ ID NO: 110; and a light chain variable region (VL) comprising a VLCDR1 amino acid sequence of SEQ ID NO: 117, a VLCDR2 amino acid sequence of SEQ ID NO: 118, and a VLCDR3 amino acid sequence of SEQ ID NO: 119, each disclosed in Table 7.

In one embodiment, the anti-LAG-3 antibody molecule comprises a VH comprising a VHCDR1 encoded by the nucleotide sequence of SEQ ID NO: 143 or 144, a VHCDR2 encoded by the nucleotide sequence of SEQ ID NO: 145 or 146, and a VHCDR3 encoded by the nucleotide sequence of SEQ ID NO: 147 or 148; and a VL comprising a VLCDR1 encoded by the nucleotide sequence of SEQ ID NO: 153 or 154, a VLCDR2 encoded by the nucleotide sequence of SEQ ID NO: 155 or 156, and a VLCDR3 encoded by the nucleotide sequence of SEQ ID NO: 157 or 158, each disclosed in Table 7. In one embodiment, the anti-LAG-3 antibody molecule comprises a VH comprising a VHCDR1 encoded by the nucleotide sequence of SEQ ID NO: 165 or 144, a VHCDR2 encoded by the nucleotide sequence of SEQ ID NO: 166 or 146, and a VHCDR3 encoded by the nucleotide sequence of SEQ ID NO: 167 or 148; and a VL comprising a VLCDR1 encoded by the nucleotide sequence of SEQ ID NO: 153 or 154, a VLCDR2 encoded by the nucleotide sequence of SEQ ID NO: 155 or 156, and a VLCDR3 encoded by the nucleotide sequence of SEQ ID NO: 157 or 158, each disclosed in Table 7.

In one embodiment, the anti-LAG-3 antibody molecule comprises a VH comprising the amino acid sequence of SEQ ID NO: 113, or an amino acid sequence at least 85%, 90%, 95%, or 99% identical or higher to SEQ ID NO: 113. In one embodiment, the anti-LAG-3 antibody molecule comprises a VL comprising the amino acid sequence of SEQ ID NO: 125, or an amino acid sequence at least 85%, 90%, 95%, or 99% identical or higher to SEQ ID NO: 125. In one embodiment, the anti-LAG-3 antibody molecule comprises a VH comprising the amino acid sequence of SEQ ID NO: 131, or an amino acid sequence at least 85%, 90%, 95%, or 99% identical or higher to SEQ ID NO: 131. In one embodiment, the anti-LAG-3 antibody molecule comprises a VL comprising the amino acid sequence of SEQ ID NO: 137, or an amino acid sequence at least 85%, 90%, 95%, or 99% identical or higher to SEQ ID NO: 137. In one embodiment, the anti-LAG-3 antibody molecule comprises a VH comprising the amino acid sequence of SEQ ID NO: 113 and a VL comprising the amino acid sequence of SEQ ID NO: 125. In one embodiment, the anti-LAG-3 antibody molecule comprises a VH comprising the amino acid sequence of SEQ ID NO: 131 and a VL comprising the amino acid sequence of SEQ ID NO: 137.

In one embodiment, the antibody molecule comprises a VH encoded by the nucleotide sequence of SEQ ID NO: 114 or 115, or a nucleotide sequence at least 85%, 90%, 95%, or 99% identical or higher to SEQ ID NO: 114 or 115. In one embodiment, the antibody molecule comprises a VL encoded by the nucleotide sequence of SEQ ID NO: 126 or 127, or a nucleotide sequence at least 85%, 90%, 95%, or 99% identical or higher to SEQ ID NO: 126 or 127. In one embodiment, the antibody molecule comprises a VH encoded by the nucleotide sequence of SEQ ID NO: 132 or 133, or a nucleotide sequence at least 85%, 90%, 95%, or 99% identical or higher to SEQ ID NO: 132 or 133. In one embodiment, the antibody molecule comprises a VL encoded by the nucleotide sequence of SEQ ID NO: 138 or 139, or a nucleotide sequence at least 85%, 90%, 95%, or 99% identical or higher to SEQ ID NO: 138 or 139. In one embodiment, the antibody molecule comprises a VH encoded by the nucleotide sequence of SEQ ID NO: 114 or 115 and a VL encoded by the nucleotide sequence of SEQ ID NO: 126 or 127. In one embodiment, the antibody molecule comprises a VH encoded by the nucleotide sequence of SEQ ID NO: 132 or 133 and a VL encoded by the nucleotide sequence of SEQ ID NO: 138 or 139.

In one embodiment, the anti-LAG-3 antibody molecule comprises a heavy chain comprising the amino acid sequence of SEQ ID NO: 116, or an amino acid sequence at least 85%, 90%, 95%, or 99% identical or higher to SEQ ID NO: 116. In one embodiment, the anti-LAG-3 antibody molecule comprises a light chain comprising the amino acid sequence of SEQ ID NO: 128, or an amino acid sequence at least 85%, 90%, 95%, or 99% identical or higher to SEQ ID NO: 128. In one embodiment, the anti-LAG-3 antibody molecule comprises a heavy chain comprising the amino acid sequence of SEQ ID NO: 134, or an amino acid sequence at least 85%, 90%, 95%, or 99% identical or higher to SEQ ID NO: 134. In one embodiment, the anti-LAG-3 antibody molecule comprises a light chain comprising the amino acid sequence of SEQ ID NO: 140, or an amino acid sequence at least 85%, 90%, 95%, or 99% identical or higher to SEQ ID NO: 140. In one embodiment, the anti-LAG-3 antibody molecule comprises a heavy chain comprising the amino acid sequence of SEQ ID NO: 116 and a light chain comprising the amino acid sequence of SEQ ID NO: 128. In one embodiment, the anti-LAG-3 antibody molecule comprises a heavy chain comprising the amino acid sequence of SEQ ID NO: 134 and a light chain comprising the amino acid sequence of SEQ ID NO: 140.

In one embodiment, the antibody molecule comprises a heavy chain encoded by the nucleotide sequence of SEQ ID NO: 123 or 124, or a nucleotide sequence at least 85%, 90%, 95%, or 99% identical or higher to SEQ ID NO: 123 or 124. In one embodiment, the antibody molecule comprises a light chain encoded by the nucleotide sequence of SEQ ID NO: 129 or 130, or a nucleotide sequence at least 85%, 90%, 95%, or 99% identical or higher to SEQ ID NO: 129 or 130. In one embodiment, the antibody molecule comprises a heavy chain encoded by the nucleotide sequence of SEQ ID NO: 135 or 136, or a nucleotide sequence at least 85%, 90%, 95%, or 99% identical or higher to SEQ ID NO: 135 or 136. In one embodiment, the antibody molecule comprises a light chain encoded by the nucleotide sequence of SEQ ID NO: 141 or 142, or a nucleotide sequence at least 85%, 90%, 95%, or 99% identical or higher to SEQ ID NO: 141 or 142. In one embodiment, the antibody molecule comprises a heavy chain encoded by the nucleotide sequence of SEQ ID NO: 123 or 124 and a light chain encoded by the nucleotide sequence of SEQ ID NO: 129 or 130. In one embodiment, the antibody molecule comprises a heavy chain encoded by the nucleotide sequence of SEQ ID NO: 135 or 136 and a light chain encoded by the nucleotide sequence of SEQ ID NO: 141 or 142.

The antibody molecules described herein can be made by vectors, host cells, and methods described in US 2015/0259420, incorporated by reference in its entirety.

TABLE 7
Amino acid and nucleotide sequences of exemplary
anti-LAG-3 antibody molecules

| | | |
|---|---|---|
| BAP050-Clone I HC | | |
| SEQ ID NO: 108 | HCDR1 | NYGMN |
| (Kabat) | | |
| SEQ ID NO: 109 | HCDR2 | WINTDTGEPTYADDFKG |
| (Kabat) | | |
| SEQ ID NO: 110 | HCDR3 | NPPYYYGTNNAEAMDY |
| (Kabat) | | |
| SEQ ID NO: 111 | HCDR1 | GFTLTNY |
| (Chothia) | | |
| SEQ ID NO: 112 | HCDR2 | NTDTGE |
| (Chothia) | | |
| SEQ ID NO: 110 | HCDR3 | NPPYYYGTNNAEAMDY |
| (Chothia) | | |
| SEQ ID NO: 113 | VH | QVQLVQSGAEVKKPGASVKVSCKASGFTLTNYGMNWVRQAR |
| | | GQRLEWIGWINTDTGEPTYADDFKGRFVFSLDTSVSTAYLQISS |
| | | LKAEDTAVYYCARNPPYYYGTNNAEAMDYWGQGTTVTVSS |
| SEQ ID NO: 114 | DNA | CAAGTGCAGCTGGTGCAGTCGGGAGCCGAAGTGAAGAAGCC |
| | VH | TGGAGCCTCGGTGAAGGTGTCGTGCAAGGCATCCGGATTCA |
| | | CCCTCACCAATTACGGGATGAACTGGGTCAGACAGGCCCGG |
| | | GGTCAACGGCTGGAGTGGATCGGATGGATTAACACCGACAC |
| | | CGGGGAGCCTACCTACGCGGACGATTTCAAGGGACGGTTCG |
| | | TGTTCTCCCTCGACACCTCCGTGTCCACCGCCTACCTCCAAA |
| | | TCTCCTCACTGAAAGCGGAGGACACCGCCGTGTACTATTGC |
| | | GCGAGGAACCCGCCCTACTACTACGGAACCAACAACGCCGA |
| | | AGCCATGGACTACTGGGGCCAGGGCACCACTGTGACTGTGT |
| | | CCAGC |
| SEQ ID NO: 115 | DNA | CAGGTGCAGCTGGTGCAGTCTGGCGCCGAAGTGAAGAAACC |
| | VH | TGGCGCCTCCGTGAAGGTGTCCTGCAAGGCCTCTGGCTTCAC |
| | | CCTGACCAACTACGGCATGAACTGGGTGCGACAGGCCAGGG |
| | | GCCAGCGGCTGGAATGGATCGGCTGGATCAACACCGACACC |
| | | GGCGAGCCTACCTACGCCGACGACTTCAAGGGCAGATTCGT |
| | | GTTCTCCCTGGACACCTCCGTGTCCACCGCCTACCTGCAGAT |
| | | CTCCAGCCTGAAGGCCGAGGATACCGCCGTGTACTACTGCG |
| | | CCCGGAACCCCCCTTACTACTACGGCACCAACAACGCCGAG |
| | | GCCATGGACTATTGGGGCCAGGGCACCACCGTGACCGTGTC |
| | | CTCT |
| SEQ ID NO: 116 | Heavy | QVQLVQSGAEVKKPGASVKVSCKASGFTLTNYGMNWVRQAR |
| | chain | GQRLEWIGWINTDTGEPTYADDFKGRFVFSLDTSVSTAYLQISS |
| | | LKAEDTAVYYCARNPPYYYGTNNAEAMDYWGQGTTVTVSSA |
| | | STKGPSVFPLAPCSRSTSESTAALGCLVKDYFPEPVTVSWNSGA |
| | | LTSGVHTFPAVLQSSGLYSLSSVVTVPSSSLGTKTYTCNVDHKP |
| | | SNTKVDKRVESKYGPPCPPCPAPEFLGGPSVFLFPPKPKDTLMIS |
| | | RTPEVTCVVVDVSQEDPEVQFNWYVDGVEVHNAKTKPREEQF |
| | | NSTYRVVSVLTVLHQDWLNGKEYKCKVSNKGLPSSIEKTISKA |
| | | KGQPREPQVYTLPPSQEEMTKNQVSLTCLVKGFYPSDIAVEWE |
| | | SNGQPENNYKTTPPVLDSDGSFFLYSRLTVDKSRWQEGNVFSC |
| | | SVMHEALHNHYTQKSLSLSLG |
| SEQ ID NO: 123 | DNA | CAAGTGCAGCTGGTGCAGTCGGGAGCCGAAGTGAAGAAGCC |
| | heavy | TGGAGCCTCGGTGAAGGTGTCGTGCAAGGCATCCGGATTCA |
| | chain | CCCTCACCAATTACGGGATGAACTGGGTCAGACAGGCCCGG |
| | | GGTCAACGGCTGGAGTGGATCGGATGGATTAACACCGACAC |
| | | CGGGGAGCCTACCTACGCGGACGATTTCAAGGGACGGTTCG |
| | | TGTTCTCCCTCGACACCTCCGTGTCCACCGCCTACCTCCAAA |
| | | TCTCCTCACTGAAAGCGGAGGACACCGCCGTGTACTATTGC |
| | | GCGAGGAACCCGCCCTACTACTACGGAACCAACAACGCCGA |
| | | AGCCATGGACTACTGGGGCCAGGGCACCACTGTGACTGTGT |
| | | CCAGCGCGTCCACTAAGGGCCCGTCCGTGTTCCCCCTGGCAC |
| | | CTTGTAGCCGGAGCACTAGCGAATCCACCGCTGCCCTCGGCT |
| | | GCCTGGTCAAGGATTACTTCCCGGAGCCCGTGACCGTGTCCT |
| | | GGAACAGCGGAGCCCTGACCTCCGGAGTGCACACCTTCCCC |
| | | GCTGTGCTGCAGAGCTCCGGGCTGTACTCGCTGTCGTCGGTG |
| | | GTCACGGTGCCTTCATCTAGCCTGGGTACCAAGACCTACACT |
| | | TGCAACGTGGACCACAAGCCTTCCAACACTAAGGTGGACAA |
| | | GCGCGTCGAATCGAAGTACGGCCCACCGTGCCCGCCTTGTC |
| | | CCGCGCCGGAGTTCCTCGGCGGTCCCTCGGTCTTTCTGTTCC |
| | | CACCGAAGCCCAAGGACACTTTGATGATTTCCCGCACCCCTG |
| | | AAGTGACATGCGTGGTCGTGGACGTGTCACAGGAAGATCCG |
| | | GAGGTGCAGTTCAATTGGTACGTGGATGGCGTCGAGGTGCA |
| | | CAACGCCAAAACCAAGCCGAGGGAGGAGCAGTTCAACTCCA |
| | | CTTACCGCGTCGTGTCCGTGCTGACGGTGCTGCATCAGGACT |
| | | GGCTGAACGGGAAGGAGTACAAGTGCAAAGTGTCCAACAA |
| | | GGGACTTCCTAGCTCAATCGAAAAGACCATCTCGAAAGCCA |
| | | AGGGACAGCCCCGGGAACCCCAAGTGTATACCCTGCCACCG |
| | | AGCCAGGAAGAAATGACTAAGAACCAAGTCTCATTGACTTG |
| | | CCTTGTGAAGGGCTTCTACCCATCGGATATCGCCGTGGAATG |
| | | GGAGTCCAACGGCCAGCCGGAAAACAACTACAAGACCACCC |
| | | CTCCGGTGCTGGACTCAGACGGATCCTTCTTCCTCTACTCGC |
| | | GGCTGACCGTGGATAAGAGCAGATGGCAGGAGGGAAATGT |
| | | GTTCAGCTGTTCTGTGATGCATGAAGCCCTGCACAACCACTA |
| | | CACTCAGAAGTCCCTGTCCCTCTCCCTGGGA |
| SEQ ID NO: 124 | DNA | CAGGTGCAGCTGGTGCAGTCTGGCGCCGAAGTGAAGAAACC |
| | heavy | TGGCGCCTCCGTGAAGGTGTCCTGCAAGGCCTCTGGCTTCAC |
| | chain | CCTGACCAACTACGGCATGAACTGGGTGCGACAGGCCAGGG |
| | | GCCAGCGGCTGGAATGGATCGGCTGGATCAACACCGACACC |
| | | GGCGAGCCTACCTACGCCGACGACTTCAAGGGCAGATTCGT |
| | | GTTCTCCCTGGACACCTCCGTGTCCACCGCCTACCTGCAGAT |
| | | CTCCAGCCTGAAGGCCGAGGATACCGCCGTGTACTACTGCG |
| | | CCCGGAACCCCCCTTACTACTACGGCACCAACAACGCCGAG |
| | | GCCATGGACTATTGGGGCCAGGGCACCACCGTGACCGTGTC |
| | | CTCTGCTTCTACCAAGGGGCCCAGCGTGTTCCCCCTGGCCCC |
| | | CTGCTCCAGAAGCACCAGCGAGAGCACAGCCGCCCTGGGCT |
| | | GCCTGGTGAAGGACTACTTCCCCGAGCCCGTGACCGTGTCCT |
| | | GGAACAGCGGAGCCCTGACCAGCGGCGTGCACACCTTCCCC |
| | | GCCGTGCTGCAGAGCAGCGGCCTGTACAGCCTGAGCAGCGT |
| | | GGTGACCGTGCCCAGCAGCAGCCTGGGCACCAAGACCTACA |
| | | CCTGTAACGTGGACCACAAGCCCAGCAACACCAAGGTGGAC |
| | | AAGAGGGTGGAGAGCAAGTACGGCCCACCCTGCCCCCCCTG |
| | | CCCAGCCCCCGAGTTCCTGGGCGGACCCAGCGTGTTCCTGTT |
| | | CCCCCCCAAGCCCAAGGACACCCTGATGATCAGCAGAACCC |
| | | CCGAGGTGACCTGTGTGGTGGTGGACGTGTCCCAGGAGGAC |
| | | CCCGAGGTCCAGTTCAACTGGTACGTGGACGGCGTGGAGGT |
| | | GCACAACGCCAAGACCAAGCCCAGAGAGGAGCAGTTTAAC |
| | | AGCACCTACCGGGTGGTGTCCGTGCTGACCGTGCTGCACCA |
| | | GGACTGGCTGAACGGCAAAGAGTACAAGTGTAAGGTCTCCA |
| | | ACAAGGGCCTGCCAAGCAGCATCGAAAAGACCATCAGCAA |
| | | GGCCAAGGGCCAGCCTAGAGAGCCCCAGGTCTACACCCTGC |
| | | CACCCAGCCAAGAGGAGATGACCAAGAACCAGGTGTCCCTG |
| | | ACCTGTCTGGTGAAGGGCTTCTACCCAAGCGACATCGCCGT |
| | | GGAGTGGGAGAGCAACGGCCAGCCCGAGAACAACTACAAG |
| | | ACCACCCCCCCAGTGCTGGACAGCGACGGCAGCTTCTTCCTG |
| | | TACAGCAGGCTGACCGTGGACAAGTCCAGATGGCAGGAGGG |
| | | CAACGTCTTTAGCTGCTCCGTGATGCACGAGGCCCTGCACAA |
| | | CCACTACACCCAGAAGAGCCTGAGCCTGTCCCTGGGC |
| BAP050-Clone I LC | | |
| SEQ ID NO: 117 | LCDR1 | SSSQDISNYLN |
| (Kabat) | | |
| SEQ ID NO: 118 | LCDR2 | YTSTLHL |
| (Kabat) | | |
| SEQ ID NO: 119 | LCDR3 | QQYYNLPWT |
| (Kabat) | | |
| SEQ ID NO: 120 | LCDR1 | SQDISNY |
| (Chothia) | | |
| SEQ ID NO: 121 | LCDR2 | YTS |
| (Chothia) | | |
| SEQ ID NO: 122 | LCDR3 | YYNLPW |
| (Chothia) | | |
| SEQ ID NO: 125 | VL | DIQMTQSPSSLSASVGDRVTITCSSSQDISNYLNWYLQKPGQSP |
| | | QLLIYYTSTLHLGVPSRFSGSGSGTEFTLTISSLQPDDFATYYCQ |
| | | QYYNLPWTFGQGTKVEIK |
| SEQ ID NO: 126 | DNA | GATATTCAGATGACTCAGTCACCTAGTAGCCTGAGCGCTAGT |
| | VL | GTGGGCGATAGAGTGACTATCACCTGTAGCTCTAGTCAGGA |
| | | TATCTCTAACTACCTGAACTGGTATCTGCAGAAGCCCGGTCA |
| | | ATCACCTCAGCTGCTGATCTACTACACTAGCACCCTGCACCT |
| | | GGGCGTGCCCTCTAGGTTTAGCGGTAGCGGTAGTGGCACCG |
| | | AGTTCACCCTGACTATCTCTAGCCTGCAGCCCGACGACTTCG |
| | | CTACCTACTACTGTCAGCAGTACTATAACCTGCCCTGGACCT |
| | | TCGGTCAAGGCACTAAGGTCGAGATTAAG |
| SEQ ID NO: 127 | DNA | GACATCCAGATGACCCAGTCCCCCTCCAGCCTGTCTGCTTCC |
| | VL | GTGGGCGACAGAGTGACCATCACCTGTTCCTCCAGCCAGGA |
| | | CATCTCCAACTACCTGAACTGGTATCTGCAGAAGCCCGGCC |
| | | AGTCCCCTCAGCTGCTGATCTACTACACCTCCACCCTGCACC |
| | | TGGGCGTGCCCTCCAGATTTTCCGGCTCTGGCTCTGGCACCG |
| | | AGTTTACCCTGACCATCAGCTCCCTGCAGCCCGACGACTTCG |
| | | CCACCTACTACTGCCAGCAGTACTACAACCTGCCCTGGACCT |
| | | TCGGCCAGGGCACCAAGGTGGAAATCAAG |
| SEQ ID NO: 128 | Light | DIQMTQSPSSLSASVGDRVTITCSSSQDISNYLNWYLQKPGQSP |
| | chain | QLLIYYTSTLHLGVPSRFSGSGSGTEFTLTISSLQPDDFATYYCQ |
| | | QYYNLPWTFGQGTKVEIKRTVAAPSVFIFPPSDEQLKSGTASVV |
| | | CLLNNFYPREAKVQWKVDNALQSGNSQESVTEQDSKDSTYSL |
| | | SSTLTLSKADYEKHKVYACEVTHQGLSSPVTKSFNRGEC |
| SEQ ID NO: 129 | DNA | GATATTCAGATGACTCAGTCACCTAGTAGCCTGAGCGCTAGT |
| | light | GTGGGCGATAGAGTGACTATCACCTGTAGCTCTAGTCAGGA |
| | chain | TATCTCTAACTACCTGAACTGGTATCTGCAGAAGCCCGGTCA |
| | | ATCACCTCAGCTGCTGATCTACTACACTAGCACCCTGCACCT |
| | | GGGCGTGCCCTCTAGGTTTAGCGGTAGCGGTAGTGGCACCG |
| | | AGTTCACCCTGACTATCTCTAGCCTGCAGCCCGACGACTTCG |
| | | CTACCTACTACTGTCAGCAGTACTATAACCTGCCCTGGACCT |
| | | TCGGTCAAGGCACTAAGGTCGAGATTAAGCGTACGGTGGCC |
| | | GCTCCCAGCGTGTTCATCTTCCCCCCCAGCGACGAGCAGCTG |
| | | AAGAGCGGCACCGCCAGCGTGGTGTGCCTGCTGAACAACTT |
| | | CTACCCCCGGGAGGCCAAGGTGCAGTGGAAGGTGGACAACG |
| | | CCCTGCAGAGCGGCAACAGCCAGGAGAGCGTCACCGAGCA |
| | | GGACAGCAAGGACTCCACCTACAGCCTGAGCAGCACCCTGA |
| | | CCCTGAGCAAGGCCGACTACGAGAAGCATAAGGTGTACGCC |
| | | TGCGAGGTGACCCACCAGGGCCTGTCCAGCCCCGTGACCAA |
| | | GAGCTTCAACAGGGGCGAGTGC |
| SEQ ID NO: 130 | DNA | GACATCCAGATGACCCAGTCCCCCTCCAGCCTGTCTGCTTCC |
| | light | GTGGGCGACAGAGTGACCATCACCTGTTCCTCCAGCCAGGA |
| | chain | CATCTCCAACTACCTGAACTGGTATCTGCAGAAGCCCGGCC |
| | | AGTCCCCTCAGCTGCTGATCTACTACACCTCCACCCTGCACC |
| | | TGGGCGTGCCCTCCAGATTTTCCGGCTCTGGCTCTGGCACCG |
| | | AGTTTACCCTGACCATCAGCTCCCTGCAGCCCGACGACTTCG |
| | | CCACCTACTACTGCCAGCAGTACTACAACCTGCCCTGGACCT |
| | | TCGGCCAGGGCACCAAGGTGGAAATCAAGCGTACGGTGGCC |
| | | GCTCCCAGCGTGTTCATCTTCCCCCCAAGCGACGAGCAGCTG |
| | | AAGAGCGGCACCGCCAGCGTGGTGTGTCTGCTGAACAACTT |
| | | CTACCCCAGGGAGGCCAAGGTGCAGTGGAAGGTGGACAAC |
| | | GCCCTGCAGAGCGGCAACAGCCAGGAGAGCGTCACCGAGC |
| | | AGGACAGCAAGGACTCCACCTACAGCCTGAGCAGCACCCTG |
| | | ACCCTGAGCAAGGCCGACTACGAGAAGCACAAGGTGTACGC |
| | | CTGTGAGGTGACCCACCAGGGCCTGTCCAGCCCCGTGACCA |
| | | AGAGCTTCAACAGGGGCGAGTGC |
| BAP050-Clone J HC | | |
| SEQ ID NO: 108 | HCDR1 | NYGMN |
| (Kabat) | | |
| SEQ ID NO: 109 | HCDR2 | WINTDTGEPTYADDFKG |
| (Kabat) | | |
| SEQ ID NO: 110 | HCDR3 | NPPYYYGTNNAEAMDY |
| (Kabat) | | |
| SEQ ID NO: 111 | HCDR1 | GFTLTNY |
| (Chothia) | | |
| SEQ ID NO: 112 | HCDR2 | NTDTGE |
| (Chothia) | | |
| SEQ ID NO: 110 | HCDR3 | NPPYYYGTNNAEAMDY |
| (Chothia) | | |
| SEQ ID NO: 131 | VH | QVQLVQSGAEVKKPGASVKVSCKASGFTLTNYGMNWVRQAP |
| | | GQGLEWMGWINTDTGEPTYADDFKGRFVFSLDTSVSTAYLQIS |
| | | SLKAEDTAVYYCARNPPYYYGTNNAEAMDYWGQGTTVTVSS |
| SEQ ID NO: 132 | DNA | CAGGTGCAGCTGGTGCAGTCAGGCGCCGAAGTGAAGAAACC |
| | VH | CGGCGCTAGTGTGAAAGTCAGCTGTAAAGCTAGTGGCTTCA |
| | | CCCTGACTAACTACGGGATGAACTGGGTCCGCCAGGCCCCA |
| | | GGTCAAGGCCTCGAGTGGATGGGCTGGATTAACACCGACAC |
| | | CGGCGAGCCTACCTACGCCGACGACTTTAAGGGCAGATTCG |
| | | TGTTTAGCCTGGACACTAGTGTGTCTACCGCCTACCTGCAGA |
| | | TCTCTAGCCTGAAGGCCGAGGACACCGCCGTCTACTACTGC |
| | | GCTAGAAACCCCCCCTACTACTACGGCACTAACAACGCCGA |
| | | GGCTATGGACTACTGGGGTCAAGGCACTACCGTGACCGTGT |
| | | CTAGC |
| SEQ ID NO: 133 | DNA | CAGGTGCAGCTGGTGCAGTCTGGCGCCGAAGTGAAGAAACC |
| | VH | TGGCGCCTCCGTGAAGGTGTCCTGCAAGGCCTCTGGCTTCAC |
| | | CCTGACCAACTACGGCATGAACTGGGTGCGACAGGCCCCTG |
| | | GACAGGGCCTGGAATGGATGGGCTGGATCAACACCGACACC |
| | | GGCGAGCCTACCTACGCCGACGACTTCAAGGGCAGATTCGT |
| | | GTTCTCCCTGGACACCTCCGTGTCCACCGCCTACCTGCAGAT |
| | | CTCCAGCCTGAAGGCCGAGGATACCGCCGTGTACTACTGCG |
| | | CCCGGAACCCCCCTTACTACTACGGCACCAACAACGCCGAG |
| | | GCCATGGACTATTGGGGCCAGGGCACCACCGTGACCGTGTC |
| | | CTCT |
| SEQ ID NO: 134 | Heavy | QVQLVQSGAEVKKPGASVKVSCKASGFTLTNYGMNWVRQAP |
| | chain | GQGLEWMGWINTDTGEPTYADDFKGRFVFSLDTSVSTAYLQIS |
| | | SLKAEDTAVYYCARNPPYYYGTNNAEAMDYWGQGTTVTVSS |
| | | ASTKGPSVFPLAPCSRSTSESTAALGCLVKDYFPEPVTVSWNSG |
| | | ALTSGVHTFPAVLQSSGLYSLSSVVTVPSSSLGTKTYTCNVDHK |
| | | PSNTKVDKRVESKYGPPCPPCPAPEFLGGPSVFLFPPKPKDTLMI |
| | | SRTPEVTCVVVDVSQEDPEVQFNWYVDGVEVHNAKTKPREEQ |
| | | FNSTYRVVSVLTVLHQDWLNGKEYKCKVSNKGLPSSIEKTISK |
| | | AKGQPREPQVYTLPPSQEEMTKNQVSLTCLVKGFYPSDIAVEW |
| | | ESNGQPENNYKTTPPVLDSDGSFFLYSRLTVDKSRWQEGNVFS |
| | | CSVMHEALHNHYTQKSLSLSLG |
| SEQ ID NO: 135 | DNA | CAGGTGCAGCTGGTGCAGTCAGGCGCCGAAGTGAAGAAACC |
| | heavy | CGGCGCTAGTGTGAAAGTCAGCTGTAAAGCTAGTGGCTTCA |
| | chain | CCCTGACTAACTACGGGATGAACTGGGTCCGCCAGGCCCCA |
| | | GGTCAAGGCCTCGAGTGGATGGGCTGGATTAACACCGACAC |
| | | CGGCGAGCCTACCTACGCCGACGACTTTAAGGGCAGATTCG |
| | | TGTTTAGCCTGGACACTAGTGTGTCTACCGCCTACCTGCAGA |
| | | TCTCTAGCCTGAAGGCCGAGGACACCGCCGTCTACTACTGC |
| | | GCTAGAAACCCCCCCTACTACTACGGCACTAACAACGCCGA |
| | | GGCTATGGACTACTGGGGTCAAGGCACTACCGTGACCGTGT |
| | | CTAGCGCTAGCACTAAGGGCCCGTCCGTGTTCCCCCTGGCAC |
| | | CTTGTAGCCGGAGCACTAGCGAATCCACCGCTGCCCTCGGCT |
| | | GCCTGGTCAAGGATTACTTCCCGGAGCCCGTGACCGTGTCCT |
| | | GGAACAGCGGAGCCCTGACCTCCGGAGTGCACACCTTCCCC |
| | | GCTGTGCTGCAGAGCTCCGGGCTGTACTCGCTGTCGTCGGTG |
| | | GTCACGGTGCCTTCATCTAGCCTGGGTACCAAGACCTACACT |
| | | TGCAACGTGGACCACAAGCCTTCCAACACTAAGGTGGACAA |
| | | GCGCGTCGAATCGAAGTACGGCCCACCGTGCCCGCCTTGTC |
| | | CCGCGCCGGAGTTCCTCGGCGGTCCCTCGGTCTTTCTGTTCC |
| | | CACCGAAGCCCAAGGACACTTTGATGATTTCCCGCACCCCTG |
| | | AAGTGACATGCGTGGTCGTGGACGTGTCACAGGAAGATCCG |
| | | GAGGTGCAGTTCAATTGGTACGTGGATGGCGTCGAGGTGCA |
| | | CAACGCCAAAACCAAGCCGAGGGAGGAGCAGTTCAACTCCA |
| | | CTTACCGCGTCGTGTCCGTGCTGACGGTGCTGCATCAGGACT |
| | | GGCTGAACGGGAAGGAGTACAAGTGCAAAGTGTCCAACAA |
| | | GGGACTTCCTAGCTCAATCGAAAAGACCATCTCGAAAGCCA |
| | | AGGGACAGCCCCGGGAACCCCAAGTGTATACCCTGCCACCG |
| | | AGCCAGGAAGAAATGACTAAGAACCAAGTCTCATTGACTTG |
| | | CCTTGTGAAGGGCTTCTACCCATCGGATATCGCCGTGGAATG |
| | | GGAGTCCAACGGCCAGCCGGAAAACAACTACAAGACCACCC |
| | | CTCCGGTGCTGGACTCAGACGGATCCTTCTTCCTCTACTCGC |
| | | GGCTGACCGTGGATAAGAGCAGATGGCAGGAGGGAAATGT |
| | | GTTCAGCTGTTCTGTGATGCATGAAGCCCTGCACAACCACTA |
| | | CACTCAGAAGTCCCTGTCCCTCTCCCTGGGA |
| SEQ ID NO: 136 | DNA | CAGGTGCAGCTGGTGCAGTCTGGCGCCGAAGTGAAGAAACC |
| | heavy | TGGCGCCTCCGTGAAGGTGTCCTGCAAGGCCTCTGGCTTCAC |
| | chain | CCTGACCAACTACGGCATGAACTGGGTGCGACAGGCCCCTG |
| | | GACAGGGCCTGGAATGGATGGGCTGGATCAACACCGACACC |
| | | GGCGAGCCTACCTACGCCGACGACTTCAAGGGCAGATTCGT |
| | | GTTCTCCCTGGACACCTCCGTGTCCACCGCCTACCTGCAGAT |
| | | CTCCAGCCTGAAGGCCGAGGATACCGCCGTGTACTACTGCG |
| | | CCCGGAACCCCCCTTACTACTACGGCACCAACAACGCCGAG |
| | | GCCATGGACTATTGGGGCCAGGGCACCACCGTGACCGTGTC |
| | | CTCTGCTTCTACCAAGGGGCCCAGCGTGTTCCCCCTGGCCCC |
| | | CTGCTCCAGAAGCACCAGCGAGAGCACAGCCGCCCTGGGCT |
| | | GCCTGGTGAAGGACTACTTCCCCGAGCCCGTGACCGTGTCCT |
| | | GGAACAGCGGAGCCCTGACCAGCGGCGTGCACACCTTCCCC |
| | | GCCGTGCTGCAGAGCAGCGGCCTGTACAGCCTGAGCAGCGT |
| | | GGTGACCGTGCCCAGCAGCAGCCTGGGCACCAAGACCTACA |
| | | CCTGTAACGTGGACCACAAGCCCAGCAACACCAAGGTGGAC |
| | | AAGAGGGTGGAGAGCAAGTACGGCCCACCCTGCCCCCCCTG |
| | | CCCAGCCCCCGAGTTCCTGGGCGGACCCAGCGTGTTCCTGTT |
| | | CCCCCCCAAGCCCAAGGACACCCTGATGATCAGCAGAACCC |
| | | CCGAGGTGACCTGTGTGGTGGTGGACGTGTCCCAGGAGGAC |
| | | CCCGAGGTCCAGTTCAACTGGTACGTGGACGGCGTGGAGGT |
| | | GCACAACGCCAAGACCAAGCCCAGAGAGGAGCAGTTTAAC |
| | | AGCACCTACCGGGTGGTGTCCGTGCTGACCGTGCTGCACCA |
| | | GGACTGGCTGAACGGCAAAGAGTACAAGTGTAAGGTCTCCA |
| | | ACAAGGGCCTGCCAAGCAGCATCGAAAAGACCATCAGCAA |
| | | GGCCAAGGGCCAGCCTAGAGAGCCCCAGGTCTACACCCTGC |
| | | CACCCAGCCAAGAGGAGATGACCAAGAACCAGGTGTCCCTG |
| | | ACCTGTCTGGTGAAGGGCTTCTACCCAAGCGACATCGCCGT |
| | | GGAGTGGGAGAGCAACGGCCAGCCCGAGAACAACTACAAG |
| | | ACCACCCCCCCAGTGCTGGACAGCGACGGCAGCTTCTTCCTG |
| | | TACAGCAGGCTGACCGTGGACAAGTCCAGATGGCAGGAGGG |
| | | CAACGTCTTTAGCTGCTCCGTGATGCACGAGGCCCTGCACAA |
| | | CCACTACACCCAGAAGAGCCTGAGCCTGTCCCTGGGC |
| BAP050-Clone J LC | | |
| SEQ ID NO: 117 | LCDR1 | SSSQDISNYLN |
| (Kabat) | | |
| SEQ ID NO: 118 | LCDR2 | YTSTLHL |
| (Kabat) | | |
| SEQ ID NO: 119 | LCDR3 | QQYYNLPWT |
| (Kabat) | | |
| SEQ ID NO: 120 | LCDR1 | SQDISNY |
| (Chothia) | | |
| SEQ ID NO: 121 | LCDR2 | YTS |
| (Chothia) | | |
| SEQ ID NO: 122 | LCDR3 | YYNLPW |
| (Chothia) | | |
| SEQ ID NO: 137 | VL | DIQMTQSPSSLSASVGDRVTITCSSSQDISNYLNWYQQKPGKAP |
| | | KLLIYYTSTLHLGIPPRFSGSGYGTDFTLTINNIESEDAAYYFCQ |
| | | QYYNLPWTFGQGTKVEIK |
| SEQ ID NO: 138 | DNA | GATATTCAGATGACTCAGTCACCTAGTAGCCTGAGCGCTAGT |
| | VL | GTGGGCGATAGAGTGACTATCACCTGTAGCTCTAGTCAGGA |
| | | TATCTCTAACTACCTGAACTGGTATCAGCAGAAGCCCGGTA |
| | | AAGCCCCTAAGCTGCTGATCTACTACACTAGCACCCTGCACC |
| | | TGGGAATCCCCCCTAGGTTTAGCGGTAGCGGCTACGGCACC |
| | | GACTTCACCCTGACTATTAACAATATCGAGTCAGAGGACGC |
| | | CGCCTACTACTTCTGTCAGCAGTACTATAACCTGCCCTGGAC |
| | | CTTCGGTCAAGGCACTAAGGTCGAGATTAAG |
| SEQ ID NO: 139 | DNA | GACATCCAGATGACCCAGTCCCCCTCCAGCCTGTCTGCTTCC |
| | VL | GTGGGCGACAGAGTGACCATCACCTGTTCCTCCAGCCAGGA |
| | | CATCTCCAACTACCTGAACTGGTATCAGCAGAAGCCCGGCA |
| | | AGGCCCCCAAGCTGCTGATCTACTACACCTCCACCCTGCACC |
| | | TGGGCATCCCCCCTAGATTCTCCGGCTCTGGCTACGGCACCG |
| | | ACTTCACCCTGACCATCAACAACATCGAGTCCGAGGACGCC |
| | | GCCTACTACTTCTGCCAGCAGTACTACAACCTGCCCTGGACC |
| | | TTCGGCCAGGGCACCAAGGTGGAAATCAAG |
| SEQ ID NO: 140 | Light | DIQMTQSPSSLSASVGDRVTITCSSSQDISNYLNWYQQKPGKAP |
| | chain | KLLIYYTSTLHLGIPPRFSGSGYGTDFTLTINNIESEDAAYYFCQ |
| | | QYYNLPWTFGQGTKVEIKRTVAAPSVFIFPPSDEQLKSGTASVV |
| | | CLLNNFYPREAKVQWKVDNALQSGNSQESVTEQDSKDSTYSL |
| | | SSTLTLSKADYEKHKVYACEVTHQGLSSPVTKSFNRGEC |
| SEQ ID NO: 141 | DNA | GATATTCAGATGACTCAGTCACCTAGTAGCCTGAGCGCTAGT |
| | light | GTGGGCGATAGAGTGACTATCACCTGTAGCTCTAGTCAGGA |
| | chain | TATCTCTAACTACCTGAACTGGTATCAGCAGAAGCCCGGTA |
| | | AAGCCCCTAAGCTGCTGATCTACTACACTAGCACCCTGCACC |
| | | TGGGAATCCCCCCTAGGTTTAGCGGTAGCGGCTACGGCACC |
| | | GACTTCACCCTGACTATTAACAATATCGAGTCAGAGGACGC |
| | | CGCCTACTACTTCTGTCAGCAGTACTATAACCTGCCCTGGAC |
| | | CTTCGGTCAAGGCACTAAGGTCGAGATTAAGCGTACGGTGG |
| | | CCGCTCCCAGCGTGTTCATCTTCCCCCCCAGCGACGAGCAGC |
| | | TGAAGAGCGGCACCGCCAGCGTGGTGTGCCTGCTGAACAAC |
| | | TTCTACCCCCGGGAGGCCAAGGTGCAGTGGAAGGTGGACAA |
| | | CGCCCTGCAGAGCGGCAACAGCCAGGAGAGCGTCACCGAGC |
| | | AGGACAGCAAGGACTCCACCTACAGCCTGAGCAGCACCCTG |
| | | ACCCTGAGCAAGGCCGACTACGAGAAGCATAAGGTGTACGC |
| | | CTGCGAGGTGACCCACCAGGGCCTGTCCAGCCCCGTGACCA |
| | | AGAGCTTCAACAGGGGCGAGTGC |
| SEQ ID NO: 142 | DNA | GACATCCAGATGACCCAGTCCCCCTCCAGCCTGTCTGCTTCC |
| | light | GTGGGCGACAGAGTGACCATCACCTGTTCCTCCAGCCAGGA |
| | chain | CATCTCCAACTACCTGAACTGGTATCAGCAGAAGCCCGGCA |
| | | AGGCCCCCAAGCTGCTGATCTACTACACCTCCACCCTGCACC |
| | | TGGGCATCCCCCCTAGATTCTCCGGCTCTGGCTACGGCACCG |
| | | ACTTCACCCTGACCATCAACAACATCGAGTCCGAGGACGCC |
| | | GCCTACTACTTCTGCCAGCAGTACTACAACCTGCCCTGGACC |
| | | TTCGGCCAGGGCACCAAGGTGGAAATCAAGCGTACGGTGGC |
| | | CGCTCCCAGCGTGTTCATCTTCCCCCCAAGCGACGAGCAGCT |
| | | GAAGAGCGGCACCGCCAGCGTGGTGTGTCTGCTGAACAACT |
| | | TCTACCCCAGGGAGGCCAAGGTGCAGTGGAAGGTGGACAAC |
| | | GCCCTGCAGAGCGGCAACAGCCAGGAGAGCGTCACCGAGC |
| | | AGGACAGCAAGGACTCCACCTACAGCCTGAGCAGCACCCTG |
| | | ACCCTGAGCAAGGCCGACTACGAGAAGCACAAGGTGTACGC |
| | | CTGTGAGGTGACCCACCAGGGCCTGTCCAGCCCCGTGACCA |
| | | AGAGCTTCAACAGGGGCGAGTGC |
| BAP050-Clone I HC | | |
| SEQ ID NO: 143 | HCDR1 | AATTACGGGATGAAC |
| (Kabat) | | |
| SEQ ID NO: 144 | HCDR1 | AACTACGGCATGAAC |
| (Kabat) | | |
| SEQ ID NO: 145 | HCDR2 | TGGATTAACACCGACACCGGGGAGCCTACCTACGCGGACGA |
| (Kabat) | | TTTCAAGGGA |
| SEQ ID NO: 146 | HCDR2 | TGGATCAACACCGACACCGGCGAGCCTACCTACGCCGACGA |
| (Kabat) | | CTTCAAGGGC |
| SEQ ID NO: 147 | HCDR3 | AACCCGCCCTACTACTACGGAACCAACAACGCCGAAGCCAT |
| (Kabat) | | GGACTAC |
| SEQ ID NO: 148 | HCDR3 | AACCCCCCTTACTACTACGGCACCAACAACGCCGAGGCCAT |
| (Kabat) | | GGACTAT |
| SEQ ID NO: 149 | HCDR1 | GGATTCACCCTCACCAATTAC |
| (Chothia) | | |
| SEQ ID NO: 150 | HCDR1 | GGCTTCACCCTGACCAACTAC |
| (Chothia) | | |
| SEQ ID NO: 151 | HCDR2 | AACACCGACACCGGGGAG |
| (Chothia) | | |
| SEQ ID NO: 152 | HCDR2 | AACACCGACACCGGCGAG |
| (Chothia) | | |
| SEQ ID NO: 147 | HCDR3 | AACCCGCCCTACTACTACGGAACCAACAACGCCGAAGCCAT |
| (Chothia) | | GGACTAC |
| SEQ ID NO: 148 | HCDR3 | AACCCCCCTTACTACTACGGCACCAACAACGCCGAGGCCAT |
| (Chothia) | | GGACTAT |
| BAP050-Clone I LC | | |
| SEQ ID NO: 153 | LCDR1 | AGCTCTAGTCAGGATATCTCTAACTACCTGAAC |
| (Kabat) | | |
| SEQ ID NO: 154 | LCDR1 | TCCTCCAGCCAGGACATCTCCAACTACCTGAAC |
| (Kabat) | | |
| SEQ ID NO: 155 | LCDR2 | TACACTAGCACCCTGCACCTG |
| (Kabat) | | |
| SEQ ID NO: 156 | LCDR2 | TACACCTCCACCCTGCACCTG |
| (Kabat) | | |
| SEQ ID NO: 157 | LCDR3 | CAGCAGTACTATAACCTGCCCTGGACC |
| (Kabat) | | |
| SEQ ID NO: 158 | LCDR3 | CAGCAGTACTACAACCTGCCCTGGACC |
| (Kabat) | | |
| SEQ ID NO: 159 | LCDR1 | AGTCAGGATATCTCTAACTAC |
| (Chothia) | | |
| SEQ ID NO: 160 | LCDR1 | AGCCAGGACATCTCCAACTAC |
| (Chothia) | | |
| SEQ ID NO: 161 | LCDR2 | TACACTAGC |
| (Chothia) | | |
| SEQ ID NO: 162 | LCDR2 | TACACCTCC |
| (Chothia) | | |
| SEQ ID NO: 163 | LCDR3 | TACTATAACCTGCCCTGG |
| (Chothia) | | |
| SEQ ID NO: 164 | LCDR3 | TACTACAACCTGCCCTGG |
| (Chothia) | | |
| BAP050-Clone J HC | | |
| SEQ ID NO: 165 | HCDR1 | AACTACGGGATGAAC |
| (Kabat) | | |
| SEQ ID NO: 144 | HCDR1 | AACTACGGCATGAAC |
| (Kabat) | | |
| SEQ ID NO: 166 | HCDR2 | TGGATTAACACCGACACCGGCGAGCCTACCTACGCCGACGA |
| (Kabat) | | CTTTAAGGGC |
| SEQ ID NO: 146 | HCDR2 | TGGATCAACACCGACACCGGCGAGCCTACCTACGCCGACGA |
| (Kabat) | | CTTCAAGGGC |
| SEQ ID NO: 167 | HCDR3 | AACCCCCCCTACTACTACGGCACTAACAACGCCGAGGCTAT |
| (Kabat) | | GGACTAC |
| SEQ ID NO: 148 | HCDR3 | AACCCCCCTTACTACTACGGCACCAACAACGCCGAGGCCAT |
| (Kabat) | | GGACTAT |
| SEQ ID NO: 168 | HCDR1 | GGCTTCACCCTGACTAACTAC |
| (Chothia) | | |
| SEQ ID NO: 150 | HCDR1 | GGCTTCACCCTGACCAACTAC |
| (Chothia) | | |
| SEQ ID NO: 151 | HCDR2 | AACACCGACACCGGGGAG |
| (Chothia) | | |
| SEQ ID NO: 152 | HCDR2 | AACACCGACACCGGCGAG |
| (Chothia) | | |
| SEQ ID NO: 167 | HCDR3 | AACCCCCCCTACTACTACGGCACTAACAACGCCGAGGCTAT |
| (Chothia) | | GGACTAC |
| SEQ ID NO: 148 | HCDR3 | AACCCCCCTTACTACTACGGCACCAACAACGCCGAGGCCAT |
| (Chothia) | | GGACTAT |
| BAP050-Clone J LC | | |
| SEQ ID NO: 153 | LCDR1 | AGCTCTAGTCAGGATATCTCTAACTACCTGAAC |
| (Kabat) | | |
| SEQ ID NO: 154 | LCDR1 | TCCTCCAGCCAGGACATCTCCAACTACCTGAAC |
| (Kabat) | | |
| SEQ ID NO: 155 | LCDR2 | TACACTAGCACCCTGCACCTG |
| (Kabat) | | |
| SEQ ID NO: 156 | LCDR2 | TACACCTCCACCCTGCACCTG |
| (Kabat) | | |
| SEQ ID NO: 157 | LCDR3 | CAGCAGTACTATAACCTGCCCTGGACC |
| (Kabat) | | |
| SEQ ID NO: 158 | LCDR3 | CAGCAGTACTACAACCTGCCCTGGACC |
| (Kabat) | | |
| SEQ ID NO: 159 | LCDR1 | AGTCAGGATATCTCTAACTAC |
| (Chothia) | | |
| SEQ ID NO: 160 | LCDR1 | AGCCAGGACATCTCCAACTAC |
| (Chothia) | | |
| SEQ ID NO: 161 | LCDR2 | TACACTAGC |
| (Chothia) | | |
| SEQ ID NO: 162 | LCDR2 | TACACCTCC |
| (Chothia) | | |
| SEQ ID NO: 163 | LCDR3 | TACTATAACCTGCCCTGG |
| (Chothia) | | |
| SEQ ID NO: 164 | LCDR3 | TACTACAACCTGCCCTGG |
| (Chothia) | | |

### Other Exemplary LAG-3 Inhibitors

In one embodiment, the LAG-3 inhibitor is an anti-LAG-3 antibody molecule. In one embodiment, the LAG-3 inhibitor is BMS-986016 (Bristol-Myers Squibb), also known as BMS986016. BMS-986016 and other anti-LAG-3 antibodies are disclosed in WO 2015/116539 and U.S. Pat. No. 9,505,839, incorporated by reference in their entirety. In one embodiment, the anti-LAG-3 antibody molecule comprises one or more of the CDR sequences (or collectively all of the CDR sequences), the heavy chain or light chain variable region sequence, or the heavy chain or light chain sequence of BMS-986016, e.g., as disclosed in Table 8.

In one embodiment, the anti-LAG-3 antibody molecule is TSR-033 (Tesaro). In one embodiment, the anti-LAG-3 antibody molecule comprises one or more of the CDR sequences (or collectively all of the CDR sequences), the heavy chain or light chain variable region sequence, or the heavy chain or light chain sequence of TSR-033.

In one embodiment, the anti-LAG-3 antibody molecule is IMP731 or GSK2831781 (GSK and Prima BioMed). IMP731 and other anti-LAG-3 antibodies are disclosed in WO 2008/132601 and U.S. Pat. No. 9,244,059, incorporated by reference in their entirety. In one embodiment, the anti-LAG-3 antibody molecule comprises one or more of the CDR sequences (or collectively all of the CDR sequences), the heavy chain or light chain variable region sequence, or the heavy chain or light chain sequence of IMP731, e.g., as disclosed in Table 8. In one embodiment, the anti-LAG-3 antibody molecule comprises one or more of the CDR sequences (or collectively all of the CDR sequences), the heavy chain or light chain variable region sequence, or the heavy chain or light chain sequence of GSK2831781.

In one embodiment, the anti-LAG-3 antibody molecule is IMP761 (Prima BioMed). In one embodiment, the anti-LAG-3 antibody molecule comprises one or more of the CDR sequences (or collectively all of the CDR sequences), the heavy chain or light chain variable region sequence, or the heavy chain or light chain sequence of IMP761.

Further known anti-LAG-3 antibodies include those described, e.g., in WO 2008/132601, WO 2010/019570, WO 2014/140180, WO 2015/116539, WO 2015/200119, WO 2016/028672, U.S. Pat. Nos. 9,244,059, 9,505,839, incorporated by reference in their entirety.

In one embodiment, the anti-LAG-3 antibody is an antibody that competes for binding with, and/or binds to the same epitope on LAG-3 as, one of the anti-LAG-3 antibodies described herein.

In one embodiment, the anti-LAG-3 inhibitor is a soluble LAG-3 protein, e.g., IMP321 (Prima BioMed), e.g., as disclosed in WO 2009/044273, incorporated by reference in its entirety.

TABLE 8
Amino acid sequences
of other exemplary anti-LAG-3 antibody molecules

| | | |
|---|---|---|
| BMS-986016 | | |
| SEQ ID NO: | Heavy | QVQLQQWGAGLLKPSETLSLTCAVYGGSFSDYYWNWIRQPPGKGLE |
| 169 | chain | WIGEINHRGSTNSNPSLKSRVTLSLDTSKNQFSLKLRSVTAADTAVYYC |
| | | AFGYSDYEYNWFDPWGQGTLVTVSSASTKGPSVFPLAPCSRSTSESTA |
| | | ALGCLVKDYFPEPVTVSWNSGALTSGVHTFPAVLQSSGLYSLSSVVTV |
| | | PSSSLGTKTYTCNVDHKPSNTKVDKRVESKYGPPCPPCPAPEFLGGPSV |
| | | FLFPPKPKDTLMISRTPEVTCVVVDVSQEDPEVQFNWYVDGVEVHNA |
| | | KTKPREEQFNSTYRVVSVLTVLHQDWLNGKEYKCKVSNKGLPSSIEKT |
| | | ISKAKGQPREPQVYTLPPSQEEMTKNQVSLTCLVKGFYPSDIAVEWESN |
| | | GQPENNYKTTPPVLDSDGSFFLYSRLTVDKSRWQEGNVFSCSVMHEAL |
| | | HNHYTQKSLSLSLGK |
| SEQ ID NO: | Light | EIVLTQSPATLSLSPGERATLSCRASQSISSYLAWYQQKPGQAPRLLIYD |
| 170 | chain | ASNRATGIPARFSGSGSGTDFTLTISSLEPEDFAVYYCQQRSNWPLTFG |
| | | QGTNLEIKRTVAAPSVFIFPPSDEQLKSGTASVVCLLNNFYPREAKVQW |
| | | KVDNALQSGNSQESVTEQDSKDSTYSLSSTLTLSKADYEKHKVYACEV |
| | | THQGLSSPVTKSFNRGEC |
| IMP731 | | |
| SEQ ID NO: | Heavy | QVQLKESGPGLVAPSQSLSITCTVSGFSLTAYGVNWVRQPPGKGLEWL |
| 171 | chain | GMIWDDGSTDYNSALKSRLSISKDNSKSQVFLKMNSLQTDDTARYYC |
| | | AREGDVAFDYWGQGTTLTVSSASTKGPSVFPLAPSSKSTSGGTAALGC |
| | | LVKDYFPEPVTVSWNSGALTSGVHTFPAVLQSSGLYSLSSVVTVPSSSL |
| | | GTQTYICNVNHKPSNTKVDKKVEPKSCDKTHTCPPCPAPELLGGPSVFL |
| | | FPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTIS |
| | | KAKGQPREPQVYTLPPSRDELTKNQVSLTCLVKGFYPSDIAVEWESNG |
| | | QPENNYKTTPPVLDSDGSFFLYSKLTVDKSRWQQGNVFSCSVMHEAL |
| | | HNHYTQKSLSLSPGK |
| SEQ ID NO: | Light | DIVMTQSPSSLAVSVGQKVTMSCKSSQSLLNGSNQKNYLAWYQQKPG |
| 172 | chain | QSPKLLVYFASTRDSGVPDRFIGSGSGTDFTLTISSVQAEDLADYFCLQ |
| | | HFGTPPTFGGGTKLEIKRTVAAPSVFIFPPSDEQLKSGTASVVCLLNNFY |

### TIM-3 Inhibitors

In certain embodiments, the inhibitor of an immune checkpoint molecule is an inhibitor of TIM-3. In some embodiments, the compounds of Formula (I) or compounds of Embodiment 16, 17, or 35, or a pharmaceutically acceptable salt, hydrate, solvate, prodrug, stereoisomer, or tautomer thereof, of the present disclosure are used in combination with a TIM-3 inhibitor to treat a disease, e.g., cancer. In some embodiments, the TIM-3 inhibitor is MGB453 (Novartis) or TSR-022 (Tesaro).

### Exemplary TIM-3 Inhibitors

In one embodiment, the TIM-3 inhibitor is an anti-TIM-3 antibody molecule. In one embodiment, the TIM-3 inhibitor is an anti-TIM-3 antibody molecule as disclosed in US 2015/0218274, published on Aug. 6, 2015, entitled “Antibody Molecules to TIM-3 and Uses Thereof,” incorporated by reference in its entirety.

In one embodiment, the anti-TIM-3 antibody molecule comprises at least one, two, three, four, five or six complementarity determining regions (CDRs) (or collectively all of the CDRs) from a heavy and light chain variable region comprising an amino acid sequence shown in Table 9 (e.g., from the heavy and light chain variable region sequences of ABTIM3-hum11 or ABTIM3-hum03 disclosed in Table 9), or encoded by a nucleotide sequence shown in Table 9. In some embodiments, the CDRs are according to the Kabat definition (e.g., as set out in Table 9). In some embodiments, the CDRs are according to the Chothia definition (e.g., as set out in Table 9). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 9, or encoded by a nucleotide sequence shown in Table 9.

In one embodiment, the anti-TIM-3 antibody molecule comprises a heavy chain variable region (VH) comprising a VHCDR1 amino acid sequence of SEQ ID NO: 174, a VHCDR2 amino acid sequence of SEQ ID NO: 175, and a VHCDR3 amino acid sequence of SEQ ID NO: 176; and a light chain variable region (VL) comprising a VLCDR1 amino acid sequence of SEQ ID NO: 183, a VLCDR2 amino acid sequence of SEQ ID NO: 184, and a VLCDR3 amino acid sequence of SEQ ID NO: 185, each disclosed in Table 9. In one embodiment, the anti-TIM-3 antibody molecule comprises a heavy chain variable region (VH) comprising a VHCDR1 amino acid sequence of SEQ ID NO: 174, a VHCDR2 amino acid sequence of SEQ ID NO: 193, and a VHCDR3 amino acid sequence of SEQ ID NO: 176; and a light chain variable region (VL) comprising a VLCDR1 amino acid sequence of SEQ ID NO: 183, a VLCDR2 amino acid sequence of SEQ ID NO: 184, and a VLCDR3 amino acid sequence of SEQ ID NO: 185, each disclosed in Table 9.

In one embodiment, the anti-TIM-3 antibody molecule comprises a VH comprising the amino acid sequence of SEQ ID NO: 179, or an amino acid sequence at least 85%, 90%, 95%, or 99% identical or higher to SEQ ID NO: 179. In one embodiment, the anti-TIM-3 antibody molecule comprises a VL comprising the amino acid sequence of SEQ ID NO: 189, or an amino acid sequence at least 85%, 90%, 95%, or 99% identical or higher to SEQ ID NO: 189. In one embodiment, the anti-TIM-3 antibody molecule comprises a VH comprising the amino acid sequence of SEQ ID NO: 195, or an amino acid sequence at least 85%, 90%, 95%, or 99% identical or higher to SEQ ID NO: 195. In one embodiment, the anti-TIM-3 antibody molecule comprises a VL comprising the amino acid sequence of SEQ ID NO: 199, or an amino acid sequence at least 85%, 90%, 95%, or 99% identical or higher to SEQ ID NO: 199. In one embodiment, the anti-TIM-3 antibody molecule comprises a VH comprising the amino acid sequence of SEQ ID NO: 179 and a VL comprising the amino acid sequence of SEQ ID NO: 189. In one embodiment, the anti-TIM-3 antibody molecule comprises a VH comprising the amino acid sequence of SEQ ID NO: 195 and a VL comprising the amino acid sequence of SEQ ID NO: 199.

In one embodiment, the antibody molecule comprises a VH encoded by the nucleotide sequence of SEQ ID NO: 180, or a nucleotide sequence at least 85%, 90%, 95%, or 99% identical or higher to SEQ ID NO: 180. In one embodiment, the antibody molecule comprises a VL encoded by the nucleotide sequence of SEQ ID NO: 190, or a nucleotide sequence at least 85%, 90%, 95%, or 99% identical or higher to SEQ ID NO: 190. In one embodiment, the antibody molecule comprises a VH encoded by the nucleotide sequence of SEQ ID NO: 196, or a nucleotide sequence at least 85%, 90%, 95%, or 99% identical or higher to SEQ ID NO: 196. In one embodiment, the antibody molecule comprises a VL encoded by the nucleotide sequence of SEQ ID NO: 200, or a nucleotide sequence at least 85%, 90%, 95%, or 99% identical or higher to SEQ ID NO: 200. In one embodiment, the antibody molecule comprises a VH encoded by the nucleotide sequence of SEQ ID NO: 180 and a VL encoded by the nucleotide sequence of SEQ ID NO: 190. In one embodiment, the antibody molecule comprises a VH encoded by the nucleotide sequence of SEQ ID NO: 196 and a VL encoded by the nucleotide sequence of SEQ ID NO: 200.

In one embodiment, the anti-TIM-3 antibody molecule comprises a heavy chain comprising the amino acid sequence of SEQ ID NO: 181, or an amino acid sequence at least 85%, 90%, 95%, or 99% identical or higher to SEQ ID NO: 181. In one embodiment, the anti-TIM-3 antibody molecule comprises a light chain comprising the amino acid sequence of SEQ ID NO: 191, or an amino acid sequence at least 85%, 90%, 95%, or 99% identical or higher to SEQ ID NO: 191. In one embodiment, the anti-TIM-3 antibody molecule comprises a heavy chain comprising the amino acid sequence of SEQ ID NO: 197, or an amino acid sequence at least 85%, 90%, 95%, or 99% identical or higher to SEQ ID NO: 197. In one embodiment, the anti-TIM-3 antibody molecule comprises a light chain comprising the amino acid sequence of SEQ ID NO: 201, or an amino acid sequence at least 85%, 90%, 95%, or 99% identical or higher to SEQ ID NO: 201. In one embodiment, the anti-TIM-3 antibody molecule comprises a heavy chain comprising the amino acid sequence of SEQ ID NO: 181 and a light chain comprising the amino acid sequence of SEQ ID NO: 191. In one embodiment, the anti-TIM-3 antibody molecule comprises a heavy chain comprising the amino acid sequence of SEQ ID NO: 197 and a light chain comprising the amino acid sequence of SEQ ID NO: 201.

In one embodiment, the antibody molecule comprises a heavy chain encoded by the nucleotide sequence of SEQ ID NO: 182, or a nucleotide sequence at least 85%, 90%, 95%, or 99% identical or higher to SEQ ID NO: 182. In one embodiment, the antibody molecule comprises a light chain encoded by the nucleotide sequence of SEQ ID NO: 192, or a nucleotide sequence at least 85%, 90%, 95%, or 99% identical or higher to SEQ ID NO: 192. In one embodiment, the antibody molecule comprises a heavy chain encoded by the nucleotide sequence of SEQ ID NO: 198, or a nucleotide sequence at least 85%, 90%, 95%, or 99% identical or higher to SEQ ID NO: 198. In one embodiment, the antibody molecule comprises a light chain encoded by the nucleotide sequence of SEQ ID NO: 202, or a nucleotide sequence at least 85%, 90%, 95%, or 99% identical or higher to SEQ ID NO: 202. In one embodiment, the antibody molecule comprises a heavy chain encoded by the nucleotide sequence of SEQ ID NO: 182 and a light chain encoded by the nucleotide sequence of SEQ ID NO: 192. In one embodiment, the antibody molecule comprises a heavy chain encoded by the nucleotide sequence of SEQ ID NO: 198 and a light chain encoded by the nucleotide sequence of SEQ ID NO: 202.

The antibody molecules described herein can be made by vectors, host cells, and methods described in US 2015/0218274, incorporated by reference in its entirety.

TABLE 9
Amino acid and nucleotide sequences of
exemplary anti-TIM-3 antibody molecules

| | | |
|---|---|---|
| ABTIM3-hum11 | | |
| SEQ ID NO: 174 | HCDR1 | SYNMH |
| (Kabat) | | |
| SEQ ID NO: 175 | HCDR2 | DIYPGNGDTSYNQKFKG |
| (Kabat) | | |
| SEQ ID NO: 176 | HCDR3 | VGGAFPMDY |
| (Kabat) | | |
| SEQ ID NO: 177 | HCDR1 | GYTFTSY |
| (Chothia) | | |
| SEQ ID NO: 178 | HCDR2 | YPGNGD |
| (Chothia) | | |
| SEQ ID NO: 176 | HCDR3 | VGGAFPMDY |
| (Chothia) | | |
| SEQ ID NO: 179 | VH | QVQLVQSGAEVKKPGSSVKVSCKASGYTFTSYNMHWVRQAPG |
| | | QGLEWMGDIYPGNGDTSYNQKFKGRVTITADKSTSTVYMELSS |
| | | LRSEDTAVYYCARVGGAFPMDYWGQGTTVTVSS |
| SEQ ID NO: 180 | DNAVH | CAGGTGCAGCTGGTGCAGTCAGGCGCCGAAGTGAAGAAACC |
| | | CGGCTCTAGCGTGAAAGTTTCTTGTAAAGCTAGTGGCTACAC |
| | | CTTCACTAGCTATAATATGCACTGGGTTCGCCAGGCCCCAGG |
| | | GCAAGGCCTCGAGTGGATGGGCGATATCTACCCCGGGAACG |
| | | GCGACACTAGTTATAATCAGAAGTTTAAGGGTAGAGTCACTA |
| | | TCACCGCCGATAAGTCTACTAGCACCGTCTATATGGAACTGA |
| | | GTTCCCTGAGGTCTGAGGACACCGCCGTCTACTACTGCGCTA |
| | | GAGTGGGCGGAGCCTTCCCTATGGACTACTGGGGTCAAGGCA |
| | | CTACCGTGACCGTGTCTAGC |
| SEQ ID NO: 181 | Heavy | QVQLVQSGAEVKKPGSSVKVSCKASGYTFTSYNMHWVRQAPG |
| | chain | QGLEWMGDIYPGNGDTSYNQKFKGRVTITADKSTSTVYMELSS |
| | | LRSEDTAVYYCARVGGAFPMDYWGQGTTVTVSSASTKGPSVFP |
| | | LAPCSRSTSESTAALGCLVKDYFPEPVTVSWNSGALTSGVHTFP |
| | | AVLQSSGLYSLSSVVTVPSSSLGTKTYTCNVDHKPSNTKVDKRV |
| | | ESKYGPPCPPCPAPEFLGGPSVFLFPPKPKDTLMISRTPEVTCV |
| | | VVDVSQEDPEVQFNWYVDGVEVHNAKTKPREEQFNSTYRVVSVL |
| | | TVLHQDWLNGKEYKCKVSNKGLPSSIEKTISKAKGQPREPQVYT |
| | | LPPSQEEMTKNQVSLTCLVKGFYPSDIAVEWESNGQPENNYKTT |
| | | PPVLDSDGSFFLYSRLTVDKSRWQEGNVFSCSVMHEALHNHYT |
| | | QKSLSLSLG |
| SEQ ID NO: 182 | DNA | CAGGTGCAGCTGGTGCAGTCAGGCGCCGAAGTGAAGAAACC |
| | heavy | CGGCTCTAGCGTGAAAGTTTCTTGTAAAGCTAGTGGCTACAC |
| | chain | CTTCACTAGCTATAATATGCACTGGGTTCGCCAGGCCCCAGG |
| | | GCAAGGCCTCGAGTGGATGGGCGATATCTACCCCGGGAACG |
| | | GCGACACTAGTTATAATCAGAAGTTTAAGGGTAGAGTCACTA |
| | | TCACCGCCGATAAGTCTACTAGCACCGTCTATATGGAACTGA |
| | | GTTCCCTGAGGTCTGAGGACACCGCCGTCTACTACTGCGCTA |
| | | GAGTGGGCGGAGCCTTCCCTATGGACTACTGGGGTCAAGGCA |
| | | CTACCGTGACCGTGTCTAGCGCTAGCACTAAGGGCCCGTCCG |
| | | TGTTCCCCCTGGCACCTTGTAGCCGGAGCACTAGCGAATCCA |
| | | CCGCTGCCCTCGGCTGCCTGGTCAAGGATTACTTCCCGGAGC |
| | | CCGTGACCGTGTCCTGGAACAGCGGAGCCCTGACCTCCGGAG |
| | | TGCACACCTTCCCCGCTGTGCTGCAGAGCTCCGGGCTGTACT |
| | | CGCTGTCGTCGGTGGTCACGGTGCCTTCATCTAGCCTGGGTA |
| | | CCAAGACCTACACTTGCAACGTGGACCACAAGCCTTCCAACA |
| | | CTAAGGTGGACAAGCGCGTCGAATCGAAGTACGGCCCACCG |
| | | TGCCCGCCTTGTCCCGCGCCGGAGTTCCTCGGCGGTCCCTCG |
| | | GTCTTTCTGTTCCCACCGAAGCCCAAGGACACTTTGATGATTT |
| | | CCCGCACCCCTGAAGTGACATGCGTGGTCGTGGACGTGTCAC |
| | | AGGAAGATCCGGAGGTGCAGTTCAATTGGTACGTGGATGGC |
| | | GTCGAGGTGCACAACGCCAAAACCAAGCCGAGGGAGGAGCA |
| | | GTTCAACTCCACTTACCGCGTCGTGTCCGTGCTGACGGTGCTG |
| | | CATCAGGACTGGCTGAACGGGAAGGAGTACAAGTGCAAAGT |
| | | GTCCAACAAGGGACTTCCTAGCTCAATCGAAAAGACCATCTC |
| | | GAAAGCCAAGGGACAGCCCCGGGAACCCCAAGTGTATACCC |
| | | TGCCACCGAGCCAGGAAGAAATGACTAAGAACCAAGTCTCA |
| | | TTGACTTGCCTTGTGAAGGGCTTCTACCCATCGGATATCGCCG |
| | | TGGAATGGGAGTCCAACGGCCAGCCGGAAAACAACTACAAG |
| | | ACCACCCCTCCGGTGCTGGACTCAGACGGATCCTTCTTCCTCT |
| | | ACTCGCGGCTGACCGTGGATAAGAGCAGATGGCAGGAGGGA |
| | | AATGTGTTCAGCTGTTCTGTGATGCATGAAGCCCTGCACAAC |
| | | CACTACACTCAGAAGTCCCTGTCCCTCTCCCTGGGA |
| SEQ ID NO: 183 | LCDR1 | RASESVEYYGTSLMQ |
| (Kabat) | | |
| SEQ ID NO: 184 | LCDR2 | AASNVES |
| (Kabat) | | |
| SEQ ID NO: 185 | LCDR3 | QQSRKDPST |
| (Kabat) | | |
| SEQ ID NO: 186 | LCDR1 | SESVEYYGTSL |
| (Chothia) | | |
| SEQ ID NO: 187 | LCDR2 | AAS |
| (Chothia) | | |
| SEQ ID NO: 188 | LCDR3 | SRKDPS |
| (Chothia) | | |
| SEQ ID NO: 189 | VL | AIQLTQSPSSLSASVGDRVTITCRASESVEYYGTSLMQWYQQKP |
| | | GKAPKLLIYAASNVESGVPSRFSGSGSGTDFTLTISSLQPEDFA |
| | | TYFCQQSRKDPSTFGGGTKVEIK |
| SEQ ID NO: 190 | DNA VL | GCTATTCAGCTGACTCAGTCACCTAGTAGCCTGAGCGCTAGT |
| | | GTGGGCGATAGAGTGACTATCACCTGTAGAGCTAGTGAATCA |
| | | GTCGAGTACTACGGCACTAGCCTGATGCAGTGGTATCAGCAG |
| | | AAGCCCGGGAAAGCCCCTAAGCTGCTGATCTACGCCGCCTCT |
| | | AACGTGGAATCAGGCGTGCCCTCTAGGTTTAGCGGTAGCGGT |
| | | AGTGGCACCGACTTCACCCTGACTATCTCTAGCCTGCAGCCC |
| | | GAGGACTTCGCTACCTACTTCTGTCAGCAGTCTAGGAAGGAC |
| | | CCTAGCACCTTCGGCGGAGGCACTAAGGTCGAGATTAAG |
| SEQ ID NO: 191 | Light | AIQLTQSPSSLSASVGDRVTITCRASESVEYYGTSLMQWYQQKPG |
| | chain | KAPKLLIYAASNVESGVPSRFSGSGSGTDFTLTISSLQPEDFATY |
| | | FCQQSRKDPSTFGGGTKVEIKRTVAAPSVFIFPPSDEQLKSGTAS |
| | | VVCLLNNFYPREAKVQWKVDNALQSGNSQESVTEQDSKDSTYS |
| | | LSSTLTLSKADYEKHKVYACEVTHQGLSSPVTKSFNRGEC |
| SEQ ID NO: 192 | DNA | GCTATTCAGCTGACTCAGTCACCTAGTAGCCTGAGCGCTAGT |
| | light | GTGGGCGATAGAGTGACTATCACCTGTAGAGCTAGTGAATCA |
| | chain | GTCGAGTACTACGGCACTAGCCTGATGCAGTGGTATCAGCAG |
| | | AAGCCCGGGAAAGCCCCTAAGCTGCTGATCTACGCCGCCTCT |
| | | AACGTGGAATCAGGCGTGCCCTCTAGGTTTAGCGGTAGCGGT |
| | | AGTGGCACCGACTTCACCCTGACTATCTCTAGCCTGCAGCCC |
| | | GAGGACTTCGCTACCTACTTCTGTCAGCAGTCTAGGAAGGAC |
| | | CCTAGCACCTTCGGCGGAGGCACTAAGGTCGAGATTAAGCGT |
| | | ACGGTGGCCGCTCCCAGCGTGTTCATCTTCCCCCCCAGCGAC |
| | | GAGCAGCTGAAGAGCGGCACCGCCAGCGTGGTGTGCCTGCT |
| | | GAACAACTTCTACCCCCGGGAGGCCAAGGTGCAGTGGAAGG |
| | | TGGACAACGCCCTGCAGAGCGGCAACAGCCAGGAGAGCGTC |
| | | ACCGAGCAGGACAGCAAGGACTCCACCTACAGCCTGAGCAG |
| | | CACCCTGACCCTGAGCAAGGCCGACTACGAGAAGCATAAGG |
| | | TGTACGCCTGCGAGGTGACCCACCAGGGCCTGTCCAGCCCCG |
| | | TGACCAAGAGCTTCAACAGGGGCGAGTGC |
| ABTIM3-hum03 | | |
| SEQ ID NO: 174 | HCDR1 | SYNMH |
| (Kabat) | | |
| SEQ ID NO: 193 | HCDR2 | DIYPGQGDTSYNQKFKG |
| (Kabat) | | |
| SEQ ID NO: 176 | HCDR3 | VGGAFPMDY |
| (Kabat) | | |
| SEQ ID NO: 177 | HCDR1 | GYTFTSY |
| (Chothia) | | |
| SEQ ID NO: 194 | HCDR2 | YPGQGD |
| (Chothia) | | |
| SEQ ID NO: 176 | HCDR3 | VGGAFPMDY |
| (Chothia) | | |
| SEQ ID NO: 195 | VH | QVQLVQSGAEVKKPGASVKVSCKASGYTFTSYNMHWVRQAPG |
| | | QGLEWIGDIYPGQGDTSYNQKFKGRATMTADKSTSTVYMELSS |
| | | LRSEDTAVYYCARVGGAFPMDYWGQGTLVTVSS |
| SEQ ID NO: 196 | DNA VH | CAGGTGCAGCTGGTGCAGTCAGGCGCCGAAGTGAAGAAACC |
| | | CGGCGCTAGTGTGAAAGTTAGCTGTAAAGCTAGTGGCTATAC |
| | | TTTCACTTCTTATAATATGCACTGGGTCCGCCAGGCCCCAGGT |
| | | CAAGGCCTCGAGTGGATCGGCGATATCTACCCCGGTCAAGGC |
| | | GACACTTCCTATAATCAGAAGTTTAAGGGTAGAGCTACTATG |
| | | ACCGCCGATAAGTCTACTTCTACCGTCTATATGGAACTGAGT |
| | | TCCCTGAGGTCTGAGGACACCGCCGTCTACTACTGCGCTAGA |
| | | GTGGGCGGAGCCTTCCCAATGGACTACTGGGGTCAAGGCACC |
| | | CTGGTCACCGTGTCTAGC |
| SEQ ID NO: 197 | Heavy | QVQLVQSGAEVKKPGASVKVSCKASGYTFTSYNMHWVRQAPG |
| | chain | QGLEWIGDIYPGQGDTSYNQKFKGRATMTADKSTSTVYMELSS |
| | | LRSEDTAVYYCARVGGAFPMDYWGQGTLVTVSSASTKGPSVFP |
| | | LAPCSRSTSESTAALGCLVKDYFPEPVTVSWNSGALTSGVHTFP |
| | | AVLQSSGLYSLSSVVTVPSSSLGTKTYTCNVDHKPSNTKVDKRV |
| | | ESKYGPPCPPCPAPEFLGGPSVFLFPPKPKDTLMISRTPEVTCV |
| | | VVDVSQEDPEVQFNWYVDGVEVHNAKTKPREEQFNSTYRVVSVL |
| | | TVLHQDWLNGKEYKCKVSNKGLPSSIEKTISKAKGQPREPQVYT |
| | | LPPSQEEMTKNQVSLTCLVKGFYPSDIAVEWESNGQPENNYKTT |
| | | PPVLDSDGSFFLYSRLTVDKSRWQEGNVFSCSVMHEALHNHYT |
| | | QKSLSLSLG |
| SEQ ID NO: 198 | DNA | CAGGTGCAGCTGGTGCAGTCAGGCGCCGAAGTGAAGAAACC |
| | heavy | CGGCGCTAGTGTGAAAGTTAGCTGTAAAGCTAGTGGCTATAC |
| | chain | TTTCACTTCTTATAATATGCACTGGGTCCGCCAGGCCCCAGGT |
| | | CAAGGCCTCGAGTGGATCGGCGATATCTACCCCGGTCAAGGC |
| | | GACACTTCCTATAATCAGAAGTTTAAGGGTAGAGCTACTATG |
| | | ACCGCCGATAAGTCTACTTCTACCGTCTATATGGAACTGAGT |
| | | TCCCTGAGGTCTGAGGACACCGCCGTCTACTACTGCGCTAGA |
| | | GTGGGCGGAGCCTTCCCAATGGACTACTGGGGTCAAGGCACC |
| | | CTGGTCACCGTGTCTAGCGCTAGCACTAAGGGCCCGTCCGTG |
| | | TTCCCCCTGGCACCTTGTAGCCGGAGCACTAGCGAATCCACC |
| | | GCTGCCCTCGGCTGCCTGGTCAAGGATTACTTCCCGGAGCCC |
| | | GTGACCGTGTCCTGGAACAGCGGAGCCCTGACCTCCGGAGTG |
| | | CACACCTTCCCCGCTGTGCTGCAGAGCTCCGGGCTGTACTCG |
| | | CTGTCGTCGGTGGTCACGGTGCCTTCATCTAGCCTGGGTACC |
| | | AAGACCTACACTTGCAACGTGGACCACAAGCCTTCCAACACT |
| | | AAGGTGGACAAGCGCGTCGAATCGAAGTACGGCCCACCGTG |
| | | CCCGCCTTGTCCCGCGCCGGAGTTCCTCGGCGGTCCCTCGGTC |
| | | TTTCTGTTCCCACCGAAGCCCAAGGACACTTTGATGATTTCCC |
| | | GCACCCCTGAAGTGACATGCGTGGTCGTGGACGTGTCACAGG |
| | | AAGATCCGGAGGTGCAGTTCAATTGGTACGTGGATGGCGTCG |
| | | AGGTGCACAACGCCAAAACCAAGCCGAGGGAGGAGCAGTTC |
| | | AACTCCACTTACCGCGTCGTGTCCGTGCTGACGGTGCTGCAT |
| | | CAGGACTGGCTGAACGGGAAGGAGTACAAGTGCAAAGTGTC |
| | | CAACAAGGGACTTCCTAGCTCAATCGAAAAGACCATCTCGAA |
| | | AGCCAAGGGACAGCCCCGGGAACCCCAAGTGTATACCCTGC |
| | | CACCGAGCCAGGAAGAAATGACTAAGAACCAAGTCTCATTG |
| | | ACTTGCCTTGTGAAGGGCTTCTACCCATCGGATATCGCCGTG |
| | | GAATGGGAGTCCAACGGCCAGCCGGAAAACAACTACAAGAC |
| | | CACCCCTCCGGTGCTGGACTCAGACGGATCCTTCTTCCTCTAC |
| | | TCGCGGCTGACCGTGGATAAGAGCAGATGGCAGGAGGGAAA |
| | | TGTGTTCAGCTGTTCTGTGATGCATGAAGCCCTGCACAACCA |
| | | CTACACTCAGAAGTCCCTGTCCCTCTCCCTGGGA |
| SEQ ID NO: 183 | LCDR1 | RASESVEYYGTSLMQ |
| (Kabat) | | |
| SEQ ID NO: 184 | LCDR2 | AASNVES |
| (Kabat) | | |
| SEQ ID NO: 185 | LCDR3 | QQSRKDPST |
| (Kabat) | | |
| SEQ ID NO: 186 | LCDR1 | SESVEYYGTSL |
| (Chothia) | | |
| SEQ ID NO: 187 | LCDR2 | AAS |
| (Chothia) | | |
| SEQ ID NO: 188 | LCDR3 | SRKDPS |
| (Chothia) | | |
| SEQ ID NO: 199 | VL | DIVLTQSPDSLAVSLGERATINCRASESVEYYGTSLMQWYQQKP |
| | | GQPPKLLIYAASNVESGVPDRFSGSGSGTDFTLTISSLQAEDVAV |
| | | YYCQQSRKDPSTFGGGTKVEIK |
| SEQ ID NO: 200 | DNA VL | GATATCGTCCTGACTCAGTCACCCGATAGCCTGGCCGTCAGC |
| | | CTGGGCGAGCGGGCTACTATTAACTGTAGAGCTAGTGAATCA |
| | | GTCGAGTACTACGGCACTAGCCTGATGCAGTGGTATCAGCAG |
| | | AAGCCCGGTCAACCCCCTAAGCTGCTGATCTACGCCGCCTCT |
| | | AACGTGGAATCAGGCGTGCCCGATAGGTTTAGCGGTAGCGGT |
| | | AGTGGCACCGACTTCACCCTGACTATTAGTAGCCTGCAGGCC |
| | | GAGGACGTGGCCGTCTACTACTGTCAGCAGTCTAGGAAGGAC |
| | | CCTAGCACCTTCGGCGGAGGCACTAAGGTCGAGATTAAG |
| SEQ ID NO: 201 | Light | DIVLTQSPDSLAVSLGERATINCRASESVEYYGTSLMQWYQQKP |
| | chain | GQPPKLLIYAASNVESGVPDRFSGSGSGTDFTLTISSLQAEDVAV |
| | | YYCQQSRKDPSTFGGGTKVEIKRTVAAPSVFIFPPSDEQLKSGTA |
| | | SVVCLLNNFYPREAKVQWKVDNALQSGNSQESVTEQDSKDSTY |
| | | SLSSTLTLSKADYEKHKVYACEVTHQGLSSPVTKSFNRGEC |
| SEQ ID NO: 202 | DNA | GATATCGTCCTGACTCAGTCACCCGATAGCCTGGCCGTCAGC |
| | light | CTGGGCGAGCGGGCTACTATTAACTGTAGAGCTAGTGAATCA |
| | chain | GTCGAGTACTACGGCACTAGCCTGATGCAGTGGTATCAGCAG |
| | | AAGCCCGGTCAACCCCCTAAGCTGCTGATCTACGCCGCCTCT |
| | | AACGTGGAATCAGGCGTGCCCGATAGGTTTAGCGGTAGCGGT |
| | | AGTGGCACCGACTTCACCCTGACTATTAGTAGCCTGCAGGCC |
| | | GAGGACGTGGCCGTCTACTACTGTCAGCAGTCTAGGAAGGAC |
| | | CCTAGCACCTTCGGCGGAGGCACTAAGGTCGAGATTAAGCGT |
| | | ACGGTGGCCGCTCCCAGCGTGTTCATCTTCCCCCCCAGCGAC |
| | | GAGCAGCTGAAGAGCGGCACCGCCAGCGTGGTGTGCCTGCT |
| | | GAACAACTTCTACCCCCGGGAGGCCAAGGTGCAGTGGAAGG |
| | | TGGACAACGCCCTGCAGAGCGGCAACAGCCAGGAGAGCGTC |
| | | ACCGAGCAGGACAGCAAGGACTCCACCTACAGCCTGAGCAG |
| | | CACCCTGACCCTGAGCAAGGCCGACTACGAGAAGCATAAGG |
| | | TGTACGCCTGCGAGGTGACCCACCAGGGCCTGTCCAGCCCCG |
| | | TGACCAAGAGCTTCAACAGGGGCGAGTGC |

### Other Exemplary TIM-3 Inhibitors

In one embodiment, the anti-TIM-3 antibody molecule is TSR-022 (AnaptysBio/Tesaro). In one embodiment, the anti-TIM-3 antibody molecule comprises one or more of the CDR sequences (or collectively all of the CDR sequences), the heavy chain or light chain variable region sequence, or the heavy chain or light chain sequence of TSR-022. In one embodiment, the anti-TIM-3 antibody molecule comprises one or more of the CDR sequences (or collectively all of the CDR sequences), the heavy chain or light chain variable region sequence, or the heavy chain or light chain sequence of APE5137 or APE5121, e.g., as disclosed in Table 10. APE5137, APE5121, and other anti-TIM-3 antibodies are disclosed in WO 2016/161270, incorporated by reference in its entirety.

In one embodiment, the anti-TIM-3 antibody molecule is the antibody clone F38-2E2. In one embodiment, the anti-TIM-3 antibody molecule comprises one or more of the CDR sequences (or collectively all of the CDR sequences), the heavy chain or light chain variable region sequence, or the heavy chain or light chain sequence of F38-2E2.

Further known anti-TIM-3 antibodies include those described, e.g., in WO 2016/111947, WO 2016/071448, WO 2016/144803, U.S. Pat. Nos. 8,552,156, 8,841,418, and 9,163,087, incorporated by reference in their entirety.

In one embodiment, the anti-TIM-3 antibody is an antibody that competes for binding with, and/or binds to the same epitope on TIM-3 as, one of the anti-TIM-3 antibodies described herein.

TABLE 10
Amino acid sequences of other exemplary
anti-TIM-3 antibody molecules

| | | |
|---|---|---|
| APE5137 | | |
| SEQ ID | VH | EVQLLESGGGLVQPGGSLRLSCAAASGFTFSSYDMSWVRQ |
| NO: 203 | | APGKGLDWVSTISGGGTYTYYQDSVKGRFTISRDNSKNTL |
| | | YLQMNSLRAEDTAVYYCASMDYWGQGTTVTVSSA |
| SEQ ID | VL | DIQMTQSPSSLSASVGDRVTITCRASQSIRRYLNWYHQKP |
| NO: 204 | | GKAPKLLIYGASTLQSGVPSRFSGSGSGTDFTLTISSLQP |
| | | EDFAVYYCQQSHSAPLTFGGGTKVEIKR |
| APE5121 | | |
| SEQ ID | VH | EVQVLESGGGLVQPGGSLRLYCVASGFTFSGSYAMSWVRQ |
| NO: 205 | | APGKGLEWVSAISGSGGSTYYADSVKGRFTISRDNSKNTL |
| | | YLQMNSLRAEDTAVYYCAKKYYVGPADYWGQGTLVTVSSG |
| SEQ ID | VL | DIVMTQSPDSLAVSLGERATINCKSSQSVLYSSNNKNYLA |
| NO: 206 | | WYQHKPGQPPKLLIYWASTRESGVPDRFSGSGSGTDFTLT |
| | | ISSLQAEDVAVYYCQQYYSSPLTFGGGTKIEVK |

### Cytokines

In yet another embodiment, the compounds of Formula (I) or compounds of Embodiment 16, 17, or 35, or a pharmaceutically acceptable salt, hydrate, solvate, prodrug, stereoisomer, or tautomer thereof, of the present disclosure are used in combination with one or more cytokines, including but not limited to, interferon, IL-2, IL-15, IL-7, or IL21. In certain embodiments, the compounds of Formula (I) or compounds of Embodiment 16, 17, or 35, or a pharmaceutically acceptable salt, hydrate, solvate, prodrug, stereoisomer, or tautomer thereof, are administered in combination with an IL-15/IL-15Ra complex. In some embodiments, the IL-15/IL-15Ra complex is selected from NIZ985 (Novartis), ATL-803 (Altor) or CYP0150 (Cytune).

### Exemplary IL-15/IL-15Ra Complexes

In one embodiment, the cytokine is IL-15 complexed with a soluble form of IL-15 receptor alpha (IL-15Ra). The IL-15/IL-15Ra complex may comprise IL-15 covalently or noncovalently bound to a soluble form of IL-15Ra. In a particular embodiment, the human IL-15 is noncovalently bonded to a soluble form of IL-15Ra. In a particular embodiment, the human IL-15 of the formulation comprises an amino acid sequence of SEQ ID NO: 207 in Table 11 or an amino acid sequence at least 85%, 90%, 95%, or 99% identical or higher to SEQ ID NO: 207, and the soluble form of human IL-15Ra comprises an amino acid sequence of SEQ ID NO: 208 in Table 11, or an amino acid sequence at least 85%, 90%, 95%, or 99% identical or higher to SEQ ID NO: 208, as described in WO 2014/066527, incorporated by reference in its entirety. The molecules described herein can be made by vectors, host cells, and methods described in WO 2007084342, incorporated by reference in its entirety.

TABLE 11
Amino acid and nucleotide sequences of
exemplary IL-15/IL-15Ra complexes
NIZ985

| | | |
|---|---|---|
| SEQ ID | Human | NWVNVISDLKKIEDLIQSMHIDATLYTESDVHPS |
| NO: 207 | IL-15 | CKVTAMKCFLLELQVISLESGDASIHDTVENLII |
| | | LANNSLSSNGNVTESGCKECEELEEKNIKEFLQS |
| | | FVHIVQMFINTS |
| SEQ ID | Human | ITCPPPMSVEHADIWVKSYSLYSRERYICNSGFK |
| NO: 208 | Soluble | RKAGTSSLTECVLNKATNVAHWTTPSLKCIRDPA |
| | IL-15Ra | LVHQRPAPPSTVTTAGVTPQPESLSPSGKEPAAS |
| | | SPSSNNTAATTAAIVPGSQLMPSKSPSTGTTEIS |
| | | SHESSHGTPSQTTAKNWELTASASHQPPGVYPQG |

### Other Exemplary IL-15 IL-15Ra Complexes

In one embodiment, the IL-15/IL-15Ra complex is ALT-803, an IL-15/IL-15Ra Fc fusion protein (IL-15N72D:IL-15RaSu/Fc soluble complex). ALT-803 is described in WO 2008/143794, incorporated by reference in its entirety. In one embodiment, the IL-15/IL-15Ra Fc fusion protein comprises the sequences as disclosed in Table 12.

In one embodiment, the IL-15/IL-15Ra complex comprises IL-15 fused to the sushi domain of IL-15Ra (CYP0150, Cytune). The sushi domain of IL-15Ra refers to a domain beginning at the first cysteine residue after the signal peptide of IL-15Ra, and ending at the fourth cysteine residue after said signal peptide. The complex of IL-15 fused to the sushi domain of IL-15Ra is described in WO 2007/04606 and WO 2012/175222, incorporated by reference in their entirety. In one embodiment, the IL-15/IL-15Ra sushi domain fusion comprises the sequences as disclosed in Table 12.

TABLE 12
Amino acid sequences of other exemplary IL-15/IL-15Ra complexes
ALT-803

| | | |
|---|---|---|
| SEQ ID NO: | IL-15N72D | NWVNVISDLKKIEDLIQSMHIDATLYTESDVHPSCKVTAMKCFL |
| 209 | | LELQVISLESGDASIHDTVENLIILANDSLSSNGNVTESGCKECEE |
| | | LEEKNIKEFLQSFVHIVQMFINTS |
| SEQ ID NO: | IL-15RaSu/ | ITCPPPMSVEHADIWVKSYSLYSRERYICNSGFKRKAGTSSLTEC |
| 210 | Fc | VLNKATNVAHWTTPSLKCIREPKSCDKTHTCPPCPAPELLGGPS |
| | | VFLFPPKPKDTLMISRTPEVTCVVVDVSHEDPEVKFNWYVDGV |
| | | EVHNAKTKPREEQYNSTYRVVSVLTVLHQDWLNGKEYKCKVS |
| | | NKALPAPIEKTISKAKGQPREPQVYTLPPSRDELTKNQVSLTCLV |
| | | KGFYPSD IAVEWESNGQPENNYKTTPPVLDSDGSFFLYSKLTVD |
| | | KSRWQQGNVFSCSVMHEALHNHYTQKSLSLSPGK |

IL-15/IL-15Ra sushi domain fusion (CYP0150)

| | | |
|---|---|---|
| SEQ ID NO: | Human IL- | NWVNVISDLKKIEDLIQSMHIDATLYTESDVHPSCKVTAMKCFL |
| 211 | 15 | LELQVISLESGDASIHDTVENLIILANNSLSSNGNVTESGCKECEE |
| | | LEXKNIKEFLQSFVHIVQMFINTS |
| | | Where X is E or K |
| SEQ ID NO: | Human IL- | ITCPPPMSVEHADIWVKSYSLYSRERYICNSGFKRKAGTSSLTEC |
| 212 | 15Ra sushi | VLNKATNVAHWTTPSLKCIRDPALVHQRPAPP |
| | and hinge | |
| | domains | |

In yet another embodiment, the compounds of Formula (I) or compounds of Embodiment 16, 17, or 35, or a pharmaceutically acceptable salt, hydrate, solvate, prodrug, stereoisomer, or tautomer thereof, of the present disclosure are used in combination with one or more agonists of toll like receptors (TLRs, e.g., TLR7, TLR8, TLR9) to treat a disease, e.g., cancer. In some embodiments, a compound of the present disclosure can be used in combination with a TLR7 agonist or a TLR7 agonist conjugate.

In some embodiments, the TLR7 agonist comprises a compound disclosed in International Application Publication No. WO2011/049677, which is hereby incorporated by reference in its entirety. In some embodiments, the TLR7 agonist comprises 3-(5-amino-2-(4-(2-(3,3-difluoro-3-phosphonopropoxy)ethoxy)-2-methylphenethyl)benzo[f][1,7]naphthyridin-8-yl)propanoic acid. In some embodiments, the TLR7 agonist comprises a compound of formula:

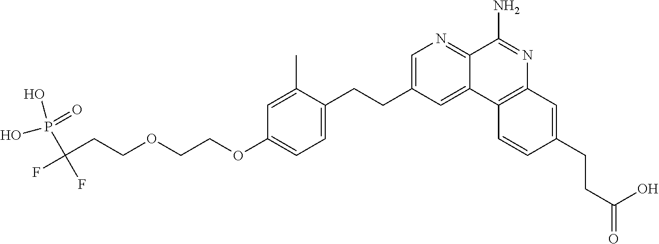 [SMILES: Cc1cc(OCCOCCC(F)(F)P(=O)(O)O)ccc1CCc1cnc2c(N)nc3cc(CCC(=O)O)ccc3c2c1]

In another embodiment, the compounds of Formula (I) or compounds of Embodiment 16, 17, or 35, or a pharmaceutically acceptable salt, hydrate, solvate, prodrug, stereoisomer, or tautomer thereof, of the present disclosure are used in combination with one or more angiogenesis inhibitors to treat cancer, e.g., Bevacizumab (Avastin®), axitinib (Inlyta®); Brivanib alaninate (BMS-582664, (S)—((R)-1-(4-(4-Fluoro-2-methyl-1H-indol-5-yloxy)-5-methylpyrrolo[2,1-f][1,2,4]triazin-6-yloxy)propan-2-yl)₂-aminopropanoate); Sorafenib (Nexavar®); Pazopanib (Votrient®); Sunitinib malate (Sutent®); Cediranib (AZD2171, CAS 288383-20-1); Vargatef (BIBF1120, CAS 928326-83-4); Foretinib (GSK1363089); Telatinib (BAY57-9352, CAS 332012-40-5); Apatinib (YN968D1, CAS 811803-05-1); Imatinib (Gleevec®); Ponatinib (AP24534, CAS 943319-70-8); Tivozanib (AV951, CAS 475108-18-0); Regorafenib (BAY73-4506, CAS 755037-03-7); Vatalanib dihydrochloride (PTK787, CAS 212141-51-0); Brivanib (BMS-540215, CAS 649735-46-6); Vandetanib (Caprelsa® or AZD6474); Motesanib diphosphate (AMG706, CAS 857876-30-3, N-(2,3-dihydro-3,3-dimethyl-1H-indol-6-yl)-2-[(4-pyridinylmethyl)amino]-3-pyridinecarboxamide, described in PCT Publication No. WO 02/066470); Dovitinib dilactic acid (TK1258, CAS 852433-84-2); Linfanib (ABT869, CAS 796967-16-3); Cabozantinib (XL184, CAS 849217-68-1); Lestaurtinib (CAS 111358-88-4); N-[5-[[[5-(1,1-Dimethylethyl)-2-oxazolyl]methyl]thio]-2-thiazolyl]-4-piperidinecarboxamide (BMS38703, CAS 345627-80-7); (3R,4R)-4-Amino-1-((4-((3-methoxyphenyl)amino)pyrrolo[2,1-f][1,2,4]triazin-5-yl)methyl)piperidin-3-ol (BMS690514); N-(3,4-Dichloro-2-fluorophenyl)-6-methoxy-7-[[(3au,50,6au)-octahydro-2-methylcyclopenta[c]pyrrol-5-yl]methoxy]-4-quinazolinamine (XL647, CAS 781613-23-8); 4-Methyl-3-[[1-methyl-6-(3-pyridinyl)-1H-pyrazolo[3,4-d]pyrimidin-4-yl]amino]-N-[3-(trifluoromethyl)phenyl]-benzamide (BHG712, CAS 940310-85-0); or Aflibercept (Eylea®).

In another embodiment, the compounds of Formula (I) or compounds of Embodiment 16, 17, or 35, or a pharmaceutically acceptable salt, hydrate, solvate, prodrug, stereoisomer, or tautomer thereof, of the present disclosure are used in combination with one or more heat shock protein inhibitors to treat cancer, e.g., Tanespimycin (17-allylamino-17-demethoxygeldanamycin, also known as KOS-953 and 17-AAG, available from SIGMA, and described in U.S. Pat. No. 4,261,989); Retaspimycin (IPI504), Ganetespib (STA-9090); [6-Chloro-9-(4-methoxy-3,5-dimethylpyridin-2-ylmethyl)-9H-purin-2-yl]amine (BIIB021 or CNF2024, CAS 848695-25-0); trans-4-[[2-(Aminocarbonyl)-5-[4,5,6,7-tetrahydro-6,6-dimethyl-4-oxo-3-(trifluoromethyl)-1H-indazol-1-yl]phenyl]amino]cyclohexyl glycine ester (SNX5422 or PF04929113, CAS 908115-27-5); 5-[2,4-Dihydroxy-5-(1-methylethyl)phenyl]-N-ethyl-4-[4-(4-morpholinylmethyl)phenyl]-3-Isoxazolecarboxamide (AUY922, CAS 747412-49-3); or 17-Dimethylaminoethylamino-17-demethoxygeldanamycin (17-DMAG).

In yet another embodiment, the compounds of Formula (I) or compounds of Embodiment 16, 17, or 35, or a pharmaceutically acceptable salt, hydrate, solvate, prodrug, stereoisomer, or tautomer thereof, of the present disclosure are used in combination with one or more HDAC inhibitors or other epigenetic modifiers. Exemplary HDAC inhibitors include, but not limited to, Voninostat (Zolinza®); Romidepsin (Istodax®); Treichostatin A (TSA); Oxamflatin; Vorinostat (Zolinza®, Suberoylanilide hydroxamic acid); Pyroxamide (syberoyl-3-aminopyridineamide hydroxamic acid); Trapoxin A (RF-1023A); Trapoxin B (RF-10238); Cyclo[(αS,2S)-α-amino-α-oxo-2-oxiraneoctanoyl-O-methyl-D-tyrosyl-L-isoleucyl-L-prolyl](Cyl-1); Cyclo[(αS,2S)-α-amino-η-oxo-2-oxiraneoctanoyl-O-methyl-D-tyrosyl-L-isoleucyl-(2S)-2-piperidinecarbonyl](Cyl-2); Cyclic[L-alanyl-D-alanyl-(2S)-η-oxo-L-α-aminooxiraneoctanoyl-D-prolyl](HC-toxin); Cyclo[(αS,2S)-α-amino-η-oxo-2-oxiraneoctanoyl-D-phenylalanyl-L-leucyl-(2S)-2-piperidinecarbonyl](WF-3161); Chlamydocin ((S)-Cyclic(2-methylalanyl-L-phenylalanyl-D-prolyl-q-oxo-L-α-aminooxiraneoctanoyl); Apicidin (Cyclo(8-oxo-L-2-aminodecanoyl-1-methoxy-L-tryptophyl-L-isoleucyl-D-2-piperidinecarbonyl); Romidepsin (Istodax®, FR-901228); 4-Phenylbutyrate; Spiruchostatin A; Mylproin (Valproic acid); Entinostat (MS-275, N-(2-Aminophenyl)-4-[N-(pyridine-3-yl-methoxycarbonyl)-amino-methyl]-benzamide); Depudecin (4,5:8,9-dianhydro-1,2,6,7,11-pentadeoxy-D-threo-D-ido-Undeca-1,6-dienitol); 4-(Acetylamino)-N-(2-aminophenyl)-benzamide (also known as CI-994); N1-(2-Aminophenyl)-N8-phenyl-octanediamide (also known as BML-210); 4-(Dimethylamino)-N-(7-(hydroxyamino)-7-oxoheptyl)benzamide (also known as M344); (E)-3-(4-(((2-(1H-indol-3-yl)ethyl)(2-hydroxyethyl)amino)-methyl)phenyl)-N-hydroxyacrylamide; Panobinostat(Farydak®); Mocetinostat, and Belinostat (also known as PXD101, Beleodaq®, or (2E)-N-Hydroxy-3-[3-(phenylsulfamoyl)phenyl]prop-2-enamide), or chidamide (also known as CS055 or HBI-8000, (E)-N-(2-amino-5-fluorophenyl)-4-((3-(pyridin-3-yl)acrylamido)methyl)benzamide). Other epigenetic modifiers include but not limited to inhibitors of EZH2 (enhancer of zeste homolog 2), EED (embryonic ectoderm development), or LSD1 (lysine-specific histone demethylase TA or KDM1A).

In yet another embodiment, the compounds of Formula (I) or compounds of Embodiment 16, 17, or 35, or a pharmaceutically acceptable salt, hydrate, solvate, prodrug, stereoisomer, or tautomer thereof, of the present disclosure are used in combination with one or more inhibitors of indoleamine-pyrrole 2,3-dioxygenase (IDO), for example, Indoximod (also known as NLG-8189), α-Cyclohexyl-5H-imidazo[5,1-a]isoindole-5-ethanol (also known as NLG919), or (4E)-4-[(3-Chloro-4-fluoroanilino)-nitrosomethylidene]-1,2,5-oxadiazol-3-amine (also known as INCB024360), to treat cancer.

### Chimeric Antigen Receptors

The present disclosure provides for the compounds of Formula (I) or compounds of Embodiment 16, 17, or 35, or a pharmaceutically acceptable salt, hydrate, solvate, prodrug, stereoisomer, or tautomer thereof for use in combination with adoptive immunotherapy methods and reagents such as chimeric antigen receptor (CAR) immune effector cells, e.g., T cells, or chimeric TCR-transduced immune effector cells, e.g., T cells. This section describes CAR technology generally that is useful in combination with the compounds of Formula (I) or compounds of Embodiment 16, 17, or 35, or a pharmaceutically acceptable salt, hydrate, solvate, prodrug, stereoisomer, or tautomer thereof, and describes CAR reagents, e.g., cells and compositions, and methods.

In general, aspects of the present disclosure pertain to or include an isolated nucleic acid molecule encoding a chimeric antigen receptor (CAR), wherein the CAR comprises an antigen binding domain (e.g., antibody or antibody fragment, TCR or TCR fragment) that binds to a tumor antigen as described herein, a transmembrane domain (e.g., a transmembrane domain described herein), and an intracellular signaling domain (e.g., an intracellular signaling domain described herein) (e.g., an intracellular signaling domain comprising a costimulatory domain (e.g., a costimulatory domain described herein) and/or a primary signaling domain (e.g., a primary signaling domain described herein). In other aspects, the present disclosure includes: host cells containing the above nucleic acids and isolated proteins encoded by such nucleic acid molecules. CAR nucleic acid constructs, encoded proteins, containing vectors, host cells, pharmaceutical compositions, and methods of administration and treatment related to the present disclosure are disclosed in detail in International Patent Application Publication No. WO2015142675, which is incorporated by reference in its entirety.

In one aspect, the disclosure pertains to an isolated nucleic acid molecule encoding a chimeric antigen receptor (CAR), wherein the CAR comprises an antigen binding domain (e.g., antibody or antibody fragment, TCR or TCR fragment) that binds to a tumor-supporting antigen (e.g., a tumor-supporting antigen as described herein), a transmembrane domain (e.g., a transmembrane domain described herein), and an intracellular signaling domain (e.g., an intracellular signaling domain described herein) (e.g., an intracellular signaling domain comprising a costimulatory domain (e.g., a costimulatory domain described herein) and/or a primary signaling domain (e.g., a primary signaling domain described herein). In some embodiments, the tumor-supporting antigen is an antigen present on a stromal cell or a myeloid-derived suppressor cell (MDSC). In other aspects, the disclosure features polypeptides encoded by such nucleic acids and host cells containing such nucleic acids and/or polypeptides.

Alternatively, aspects of the disclosure pertain to isolated nucleic acid encoding a chimeric T cell receptor (TCR) comprising a TCR alpha and/or TCR beta variable domain with specificity for a cancer antigen described herein. See for example, Dembic et al., Nature, 320, 232-238 (1986), Schumacher, Nat. Rev. Immunol., 2, 512-519 (2002), Kershaw et al., Nat. Rev. Immunol., 5, 928-940 (2005), Xue et al., Clin. Exp. Immunol., 139, 167-172 (2005), Rossig et al., Mol. Ther., 10, 5-18 (2004), and Murphy et al., Immunity, 22, 403-414 (2005); (Morgan et al. J. Immunol., 171, 3287-3295 (2003), Hughes et al., Hum. Gene Ther., 16, 1-16 (2005), Zhao et al., J. Immunol., 174, 4415-4423 (2005), Roszkowski et al., Cancer Res., 65, 1570-1576 (2005), and Engels et al., Hum. Gene Ther., 16, 799-810 (2005); US2009/03046557, the contents of which are hereby incorporated by reference in their entirety. Such chimeric TCRs may recognize, for example, cancer antigens such as MART-1, gp-100, p53, and NY-ESO-1, MAGE A3/A6, MAGEA3, SSX2, HPV-16 E6 or HPV-16 E7. In other aspects, the disclosure features polypeptides encoded by such nucleic acids and host cells containing such nucleic acids and/or polypeptides.

Sequences of non-limiting examples of various components that can be part of a CAR are listed in Table 11a, where “aa” stands for amino acids, and “na” stands for nucleic acids that encode the corresponding peptide.

TABLE 11a
Sequences of various components of CAR
(aa-amino acid sequence, na-nucleic acid sequence).

| SEQ ID | | |
|---|---|---|
| NO: | description | Sequence |
| SEQ ID | EF-1 | CGTGAGGCTCCGGTGCCCGTCAGTGGGCAGAGCGCACATCGC |
| NO: 270 | promoter | CCACAGTCCCCGAGAAGTTGGGGGGAGGGGTCGGCAATTGAA |
| | (na) | CCGGTGCCTAGAGAAGGTGGCGCGGGGTAAACTGGGAAAGTG |
| | | ATGTCGTGTACTGGCTCCGCCTTTTTCCCGAGGGTGGGGGAGA |
| | | ACCGTATATAAGTGCAGTAGTCGCCGTGAACGTTCTTTTTCGC |
| | | AACGGGTTTGCCGCCAGAACACAGGTAAGTGCCGTGTGTGGT |
| | | TCCCGCGGGCCTGGCCTCTTTACGGGTTATGGCCCTTGCGTGC |
| | | CTTGAATTACTTCCACCTGGCTGCAGTACGTGATTCTTGATCCC |
| | | GAGCTTCGGGTTGGAAGTGGGTGGGAGAGTTCGAGGCCTTGC |
| | | GCTTAAGGAGCCCCTTCGCCTCGTGCTTGAGTTGAGGCCTGGC |
| | | CTGGGCGCTGGGGCCGCCGCGTGCGAATCTGGTGGCACCTTCG |
| | | CGCCTGTCTCGCTGCTTTCGATAAGTCTCTAGCCATTTAAAATT |
| | | TTTGATGACCTGCTGCGACGCTTTTTTTCTGGCAAGATAGTCTT |
| | | GTAAATGCGGGCCAAGATCTGCACACTGGTATTTCGGTTTTTG |
| | | GGGCCGCGGGCGGCGACGGGGCCCGTGCGTCCCAGCGCACAT |
| | | GTTCGGCGAGGCGGGGCCTGCGAGCGCGGCCACCGAGAATCG |
| | | GACGGGGGTAGTCTCAAGCTGGCCGGCCTGCTCTGGTGCCTGG |
| | | CCTCGCGCCGCCGTGTATCGCCCCGCCCTGGGCGGCAAGGCTG |
| | | GCCCGGTCGGCACCAGTTGCGTGAGCGGAAAGATGGCCGCTT |
| | | CCCGGCCCTGCTGCAGGGAGCTCAAAATGGAGGACGCGGCGC |
| | | TCGGGAGAGCGGGCGGGTGAGTCACCCACACAAAGGAAAAG |
| | | GGCCTTTCCGTCCTCAGCCGTCGCTTCATGTGACTCCACGGAG |
| | | TACCGGGCGCCGTCCAGGCACCTCGATTAGTTCTCGAGCTTTT |
| | | GGAGTACGTCGTCTTTAGGTTGGGGGGAGGGGTTTTATGCGAT |
| | | GGAGTTTCCCCACACTGAGTGGGTGGAGACTGAAGTTAGGCC |
| | | AGCTTGGCACTTGATGTAATTCTCCTTGGAATTTGCCCTTTTTG |
| | | AGTTTGGATCTTGGTTCATTCTCAAGCCTCAGACAGTGGTTCA |
| | | AAGTTTTTTTCTTCCATTTCAGGTGTCGTGA |
| SEQ ID | Leader (aa) | MALPVTALLLPLALLLHAARP |
| NO: 268 | | |
| SEQ ID | Leader (na) | ATGGCCCTGCCTGTGACAGCCCTGCTGCTGCCTCTGGCTCTGC |
| NO: | | TGCTGCATGCCGCTAGACCC |
| 287 | | |
| SEQ ID | Leader (na) | ATGGCCCTCCCTGTCACCGCCCTGCTGCTTCCGCTGGCTCTTCT |
| NO: | | GCTCCACGCCGCTCGGCCC |
| 288 | | |
| SEQ ID | CD 8 hinge | TTTPAPRPPTPAPTIASQPLSLRPEACRPAAGGAVHTRGLDFACD |
| NO: 250 | (aa) | |
| SEQ ID | CD8 hinge | ACCACGACGCCAGCGCCGCGACCACCAACACCGGCGCCCACC |
| NO: 254 | (na) | ATCGCGTCGCAGCCCCTGTCCCTGCGCCCAGAGGCGTGCCGGC |
| | | CAGCGGCGGGGGGCGCAGTGCACACGAGGGGGCTGGACTTCG |
| | | CCTGTGAT |
| SEQ ID | IgG4 hinge | ESKYGPPCPPCPAPEFLGGPSVFLFPPKPKDTLMISRTPEVTCVVV |
| NO: 253 | (aa) | DVSQEDPEVQFNWYVDGVEVHNAKTKPREEQFNSTYRVVSVLT |
| | | VLHQDWLNGKEYKCKVSNKGLPSSIEKTISKAKGQPREPQVYTL |
| | | PPSQEEMTKNQVSLTCLVKGFYPSDIAVEWESNGQPENNYKTTPP |
| | | VLDSDGSFFLYSRLTVDKSRWQEGNVFSCSVMHEALHNHYTQK |
| | | SLSLSLGKM |
| SEQ ID | IgG4 hinge | GAGAGCAAGTACGGCCCTCCCTGCCCCCCTTGCCCTGCCCCCG |
| NO: 255 | (na) | AGTTCCTGGGCGGACCCAGCGTGTTCCTGTTCCCCCCCAAGCC |
| | | CAAGGACACCCTGATGATCAGCCGGACCCCCGAGGTGACCTG |
| | | TGTGGTGGTGGACGTGTCCCAGGAGGACCCCGAGGTCCAGTT |
| | | CAACTGGTACGTGGACGGCGTGGAGGTGCACAACGCCAAGAC |
| | | CAAGCCCCGGGAGGAGCAGTTCAATAGCACCTACCGGGTGGT |
| | | GTCCGTGCTGACCGTGCTGCACCAGGACTGGCTGAACGGCAA |
| | | GGAATACAAGTGTAAGGTGTCCAACAAGGGCCTGCCCAGCAG |
| | | CATCGAGAAAACCATCAGCAAGGCCAAGGGCCAGCCTCGGGA |
| | | GCCCCAGGTGTACACCCTGCCCCCTAGCCAAGAGGAGATGAC |
| | | CAAGAACCAGGTGTCCCTGACCTGCCTGGTGAAGGGCTTCTAC |
| | | CCCAGCGACATCGCCGTGGAGTGGGAGAGCAACGGCCAGCCC |
| | | GAGAACAACTACAAGACCACCCCCCCTGTGCTGGACAGCGAC |
| | | GGCAGCTTCTTCCTGTACAGCCGGCTGACCGTGGACAAGAGCC |
| | | GGTGGCAGGAGGGCAACGTCTTTAGCTGCTCCGTGATGCACG |
| | | AGGCCCTGCACAACCACTACACCCAGAAGAGCCTGAGCCTGT |
| | | CCCTGGGCAAGATG |
| SEQ ID | IgD hinge | RWPESPKAQASSVPTAQPQAEGSLAKATTAPATTRNTGRGGEEK |
| NO: 256 | (aa) | KKEKEKEEQEERETKTPECPSHTQPLGVYLLTPAVQDLWLRDKA |
| | | TFTCFVVGSDLKDAHLTWEVAGKVPTGGVEEGLLERHSNGSQSQ |
| | | HSRLTLPRSLWNAGTSVTCTLNHPSLPPQRLMALREPAAQAPVK |
| | | LSLNLLASSDPPEAASWLLCEVSGFSPPNILLMWLEDQREVNTSG |
| | | FAPARPPPQPGSTTFWAWSVLRVPAPPSPQPATYTCVVSHEDSRT |
| | | LLNASRSLEVSYVTDH |
| SEQ ID | IgD hinge | AGGTGGCCCGAAAGTCCCAAGGCCCAGGCATCTAGTGTTCCT |
| NO: 257 | (na) | ACTGCACAGCCCCAGGCAGAAGGCAGCCTAGCCAAAGCTACT |
| | | ACTGCACCTGCCACTACGCGCAATACTGGCCGTGGCGGGGAG |
| | | GAGAAGAAAAAGGAGAAAGAGAAAGAAGAACAGGAAGAGA |
| | | GGGAGACCAAGACCCCTGAATGTCCATCCCATACCCAGCCGC |
| | | TGGGCGTCTATCTCTTGACTCCCGCAGTACAGGACTTGTGGCT |
| | | TAGAGATAAGGCCACCTTTACATGTTTCGTCGTGGGCTCTGAC |
| | | CTGAAGGATGCCCATTTGACTTGGGAGGTTGCCGGAAAGGTA |
| | | CCCACAGGGGGGGTTGAGGAAGGGTTGCTGGAGCGCCATTCC |
| | | AATGGCTCTCAGAGCCAGCACTCAAGACTCACCCTTCCGAGAT |
| | | CCCTGTGGAACGCCGGGACCTCTGTCACATGTACTCTAAATCA |
| | | TCCTAGCCTGCCCCCACAGCGTCTGATGGCCCTTAGAGAGCCA |
| | | GCCGCCCAGGCACCAGTTAAGCTTAGCCTGAATCTGCTCGCCA |
| | | GTAGTGATCCCCCAGAGGCCGCCAGCTGGCTCTTATGCGAAGT |
| | | GTCCGGCTTTAGCCCGCCCAACATCTTGCTCATGTGGCTGGAG |
| | | GACCAGCGAGAAGTGAACACCAGCGGCTTCGCTCCAGCCCGG |
| | | CCCCCACCCCAGCCGGGTTCTACCACATTCTGGGCCTGGAGTG |
| | | TCTTAAGGGTCCCAGCACCACCTAGCCCCCAGCCAGCCACATA |
| | | CACCTGTGTTGTGTCCCATGAAGATAGCAGGACCCTGCTAAAT |
| | | GCTTCTAGGAGTCTGGAGGTTTCCTACGTGACTGACCATT |
| SEQ ID | GS | GGGGSGGGGS |
| NO: 258 | hinge/linker | |
| | (aa) | |
| SEQ ID | GS | GGTGGCGGAGGTTCTGGAGGTGGAGGTTCC |
| NO: 259 | hinge/linker | |
| | (aa) | |
| SEQ ID | CD8 | IYIWAPLAGTCGVLLLSLVITLYC |
| NO: 251 | transmembrane | |
| | (aa) | |
| SEQ ID | CD8 | ATCTACATCTGGGCGCCCTTGGCCGGGACTTGTGGGGTCCTTC |
| NO: 252 | transmembrane | TCCTGTCACTGGTTATCACCCTTTACTGC |
| | (na) | |
| SEQ ID | CD8 | ATCTACATTTGGGCCCCTCTGGCTGGTACTTGCGGGGTCCTGC |
| NO: 289 | transmembrane | TGCTTTCACTCGTGATCACTCTTTACTGT |
| | (na) | |
| SEQ ID | 4-1BB | KRGRKKLLYIFKQPFMRPVQTTQEEDGCSCRFPEEEEGGCEL |
| NO: 264 | intracellular | |
| | domain (aa) | |
| SEQ ID | 4-1BB | AAACGGGGCAGAAAGAAACTCCTGTATATATTCAAACAACCA |
| NO: 266 | intracellular | TTTATGAGACCAGTACAAACTACTCAAGAGGAAGATGGCTGT |
| | domain (na) | AGCTGCCGATTTCCAGAAGAAGAAGAAGGAGGATGTGAACTG |
| SEQ ID | 4-1BB | AAGCGCGGTCGGAAGAAGCTGCTGTACATCTTTAAGCAACCC |
| NO: 290 | intracellular | TTCATGAGGCCTGTGCAGACTACTCAAGAGGAGGACGGCTGT |
| | domain (na) | TCATGCCGGTTCCCAGAGGAGGAGGAAGGCGGCTGCGAACTG |
| SEQ ID | CD27 (aa) | QRRKYRSNKGESPVEPAEPCRYSCPREEEGSTIPIQEDYRKPEPAC |
| NO: 265 | | SP |
| SEQ ID | CD27 (na) | Caacgaaggaaatatagatcaaacaaaggagaaagtcctgtggagcctgcagagccttgtcgttaca |
| NO: 267 | | gctgccccagggaggaggagggcagcaccatccccatccaggaggattaccgaaaaccggagcct |
| | | gcctgctccccc |
| SEQ ID | CD3-zeta | RVKFSRSADAPAYKQGQNQLYNELNLGRREEYDVLDKRRGRDP |
| NO: 260 | (aa) | EMGGKPRRKNPQEGLYNELQKDKMAEAYSEIGMKGERRRGKG |
| | | HDGLYQGLSTATKDTYDALHMQALPPR |
| SEQ ID | CD3-zeta | AGAGTGAAGTTCAGCAGGAGCGCAGACGCCCCCGCGTACAAG |
| NO: 262 | (na) | CAGGGCCAGAACCAGCTCTATAACGAGCTCAATCTAGGACGA |
| | | AGAGAGGAGTACGATGTTTTGGACAAGAGACGTGGCCGGGAC |
| | | CCTGAGATGGGGGGAAAGCCGAGAAGGAAGAACCCTCAGGA |
| | | AGGCCTGTACAATGAACTGCAGAAAGATAAGATGGCGGAGGC |
| | | CTACAGTGAGATTGGGATGAAAGGCGAGCGCCGGAGGGGCAA |
| | | GGGGCACGATGGCCTTTACCAGGGTCTCAGTACAGCCACCAA |
| | | GGACACCTACGACGCCCTTCACATGCAGGCCCTGCCCCCTCGC |
| SEQ ID | CD3-zeta | CGCGTGAAATTCAGCCGCAGCGCAGATGCTCCAGCCTACAAG |
| NO: 291 | (na) | CAGGGGCAGAACCAGCTCTACAACGAACTCAATCTTGGTCGG |
| | | AGAGAGGAGTACGACGTGCTGGACAAGCGGAGAGGACGGGA |
| | | CCCAGAAATGGGCGGGAAGCCGCGCAGAAAGAATCCCCAAG |
| | | AGGGCCTGTACAACGAGCTCCAAAAGGATAAGATGGCAGAAG |
| | | CCTATAGCGAGATTGGTATGAAAGGGGAACGCAGAAGAGGCA |
| | | AAGGCCACGACGGACTGTACCAGGGACTCAGCACCGCCACCA |
| | | AGGACACCTATGACGCTCTTCACATGCAGGCCCTGCCGCCTCG |
| | | G |
| SEQ ID | CD3-zeta | RVKFSRSADAPAYQQGQNQLYNELNLGRREEYDVLDKRRGRDP |
| NO: 261 | (aa) | EMGGKPRRKNPQEGLYNELQKDKMAEAYSEIGMKGERRRGKG |
| | | HDGLYQGLSTATKDTYDALHMQALPPR |
| SEQ ID | CD3-zeta | AGAGTGAAGTTCAGCAGGAGCGCAGACGCCCCCGCGTACCAG |
| NO: 263 | (na) | CAGGGCCAGAACCAGCTCTATAACGAGCTCAATCTAGGACGA |
| | | AGAGAGGAGTACGATGTTTTGGACAAGAGACGTGGCCGGGAC |
| | | CCTGAGATGGGGGGAAAGCCGAGAAGGAAGAACCCTCAGGA |
| | | AGGCCTGTACAATGAACTGCAGAAAGATAAGATGGCGGAGGC |
| | | CTACAGTGAGATTGGGATGAAAGGCGAGCGCCGGAGGGGCAA |
| | | GGGGCACGATGGCCTTTACCAGGGTCTCAGTACAGCCACCAA |
| | | GGACACCTACGACGCCCTTCACATGCAGGCCCTGCCCCCTCGC |
| SEQ ID | Linker (aa) | GGGGS |
| NO: 292 | | |
| SEQ ID | PD-1 | Pgwfldspdrpwnpptfspallvvtegdnatftcsfsntsesfylnwyrmspsnqtdklaafpedrs |
| NO: 293 | extracellular | qpgqdcrfrvtqlpngrdfhmsvvrarrndsgtylcgaislapkaqikeslraelrvterraevptahp |
| | domain (aa) | spsprpagqfqtiv |
| SEQ ID | PD-1 | Cccggatggtttctggactctccggatcgcccgtggaatcccccaaccttctcaccggcactcttggttg |
| NO: 294 | extracellular | tgactgagggcgataatgcgaccttcacgtgctcgttctccaacacctccgaatcattcgtgctgaactg |
| | domain (na) | gtaccgcatgagcccgtcaaaccagaccgacaagctcgccgcgtttccggaagatcggtcgcaaccg |
| | | ggacaggattgtcggttccgcgtgactcaactgccgaatggcagagacttccacatgagcgtggtccg |
| | | cgctaggcgaaacgactccgggacctacctgtgcggagccatctcgctggcgcctaaggcccaaatc |
| | | aaagagagcttgagggccgaactgagagtgaccgagcgcagagctgaggtgccaactgcacatcca |
| | | tccccatcgcctcggcctgcggggcagtttcagaccctggtc |
| SEQ ID | PD-1 CAR | Malpvtalllplalllhaarppgwfldspdrpwnpptfspallvvtegdnatftcsfsntsesfylnwy |
| NO: 295 | (aa) with | rmspsnqtdklaafpedrsqpgqdcrfrvtqlpngrdfhmsvvrarrndsgtylcgaislapkaqik |
| | signal | eslraelmterraevptahpspsprpagqfqtivtapaprpptpaptiasqp1s1rpeacrpaaggavh |
| | | trglodfacdiyiwaplagtcgv111slvitlyckrgrkkllyifkqpfmrpvqttqeedgcscrfpeeee |
| | | ggcelrvkfsrsadapaykqgqnqlynelnlgrreeydvldkrrgrdpemggkprrknpqeglyn |
| | | elqkdkmaeayseigmkgeragkghdglyqglstatkdtydalhmqalppr |
| SEQ ID | PD-1 CAR | Atggccctccctgtcactgccctgcttctccccctcgcactcctgctccacgccgctagaccacccgga |
| NO: 296 | (na) | tggtactggactctccggatcgcccgtggaatcccccaaccttctcaccggcactcttggttgtgactga |
| | | gggcgataatgcgaccttcacgtgctcgttctccaacacctccgaatcattcgtgctgaactggtaccgc |
| | | atgagcccgtcaaaccagaccgacaagctcgccgcgtttccggaagatcggtcgcaaccgggacag |
| | | gattgtcggttccgcgtgactcaactgccgaatggcagagacttccacatgagcgtggtccgcgctagg |
| | | cgaaacgactccgggacctacctgtgcggagccatctcgctggcgcctaaggcccaaatcaaagaga |
| | | gcttgagggccgaactgagagtgaccgagcgcagagctgaggtgccaactgcacatccatccccatc |
| | | gcctcggcctgcggggcagtttcagaccctggtcacgaccactccggcgccgcgcccaccgactccg |
| | | gccccaactatcgcgagccagcccctgtcgctgaggccggaagcatgccgccctgccgccggaggt |
| | | gctgtgcatacccggggattggacttcgcatgcgacatctacatttgggctcctctcgccggaacttgtg |
| | | gcgtgctccttctgtccctggtcatcaccctgtactgcaagcggggtcggaaaaagcttctgtacattttc |
| | | aagcagcccttcatgaggcccgtgcaaaccacccaggaggaggacggttgctcctgccggttccccg |
| | | aagaggaagaaggaggttgcgagctgcgcgtgaagttctcccggagcgccgacgcccccgcctata |
| | | agcagggccagaaccagctgtacaacgaactgaacctgggacggcgggaagagtacgatgtgctgg |
| | | acaagcggcgcggccgggaccccgaaatgggcgggaagcctagaagaaagaaccctcaggaagg |
| | | cctgtataacgagctgcagaaggacaagatggccgaggcctactccgaaattgggatgaagggagag |
| | | cggcggaggggaaaggggcacgacggcctgtaccaaggactgtccaccgccaccaaggacacata |
| | | cgatgccctgcacatgcaggcccttccccctcgc |
| SEQ ID | Linker (aa) | (Gly-Gly-Gly-Ser)_{n,} where n = 1-10 |
| NO: 297 | | |
| SEQ ID | Linker (aa) | (Gly₄Ser)₄ |
| NO: 215 | | |
| SEQ ID | Linker (aa) | (Gly₄Ser)₃ |
| NO: 216 | | |
| SEQ ID | Linker (aa) | (Gly3Ser) |
| NO: 297 | | |
| SEQ ID | poly A (na) | [a]₅₀₋₅₀₀₀ |
| NO: 298 | | |
| SEQ ID | PD1 CAR | Pgwfldspdrpwnpptfspallvvtegdnatftcsfsntsesfylnwyrmspsnqtdklaafpedrs |
| NO: 299 | (aa) | qpgqdcrfmtqlpngrdfhmsvvrarmdsgtylcgaislapkaqikeslraelmterraevptahp |
| | | spsprpagqfqtlytttpaprpptpaptiasqp1slipeacrpaaggavhtrglcIfacdiyiwaplagtc |
| | | gvlllslvitlyckrgrkkllyifkqpfmrpvqttqeedgcscrfpeeeeggcelrafsrsadapayk |
| | | qgqnqlynelnlgrreeydvldkrrgrdpemggkprrknpqeglynelqkdkmaeayseigmk |
| | | geragkghdglyqglstatkdtydalhmqalppr |
| SEQ ID | ICOS | TKKKYSSSVHDPNGEYMFMRAVNTAKKSRLTDVTL |
| NO: 300 | intracellular | |
| | domain (aa) | |
| SEQ ID | ICOS | ACAAAAAAGAAGTATTCATCCAGTGTGCACGACCCTAACGGT |
| NO: 301 | intracellular | GAATACATGTTCATGAGAGCAGTGAACACAGCCAAAAAATCC |
| | domain (na) | AGACTCACAGATGTGACCCTA |
| SEQ ID | ICOSTM | TTTPAPRPPTPAPTIASQPLSLRPEACRPAAGGAVHTRGLDFACDF |
| NO: 302 | domain (aa) | WLPIGCAAFVVVCILGCILICWL |
| SEQ ID | ICOSTM | ACCACGACGCCAGCGCCGCGACCACCAACACCGGCGCCCACC |
| NO: 303 | domain (na) | ATCGCGTCGCAGCCCCTGTCCCTGCGCCCAGAGGCGTGCCGGC |
| | | CAGCGGCGGGGGGCGCAGTGCACACGAGGGGGCTGGACTTCG |
| | | CCTGTGATTTCTGGTTACCCATAGGATGTGCAGCCTTTGTTGTA |
| | | GTCTGCATTTTGGGATGCATACTTATTTGTTGGCTT |
| SEQ ID | CD28 | RSKRSRLLHSDYMNMTPRRPGPTRKHYQPYAPPRDFAAYRS |
| NO: 304 | intracellular | |
| | domain (aa) | |
| SEQ ID | CD28 | AGGAGTAAGAGGAGCAGGCTCCTGCACAGTGACTACATGAAC |
| NO: 305 | intracellular | ATGACTCCCCGCCGCCCCGGGCCCACCCGCAAGCATTACCAGC |
| | domain (na) | CCTATGCCCCACCACGCGACTTCGCAGCCTATCGCTCC |

### Targets

The present disclosure provides cells, e.g., immune effector cells (e.g., T cells, NK cells), that comprise or at any time comprised a gRNA molecule or CRISPR system as described herein, that are further engineered to contain one or more CARs that direct the immune effector cells to undesired cells (e.g., cancer cells). This is achieved through an antigen binding domain on the CAR that is specific for a cancer associated antigen. There are two classes of cancer associated antigens (tumor antigens) that can be targeted by the CARs of the instant disclosure: (1) cancer associated antigens that are expressed on the surface of cancer cells; and (2) cancer associated antigens that itself is intracellular, however, a fragment of such antigen (peptide) is presented on the surface of the cancer cells by MHC (major histocompatibility complex).

In some embodiments, the tumor antigen is chosen from one or more of: CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDGlcp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WTi); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; protein; surviving; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1).

A CAR described herein can comprise an antigen binding domain (e.g., antibody or antibody fragment, TCR or TCR fragment) that binds to a tumor-supporting antigen (e.g., a tumor-supporting antigen as described herein). In some embodiments, the tumor-supporting antigen is an antigen present on a stromal cell or a myeloid-derived suppressor cell (MDSC). Stromal cells can secrete growth factors to promote cell division in the microenvironment. MDSC cells can inhibit T cell proliferation and activation. Without wishing to be bound by theory, in some embodiments, the CAR-expressing cells destroy the tumor-supporting cells, thereby indirectly inhibiting tumor growth or survival.

In embodiments, the stromal cell antigen is chosen from one or more of: bone marrow stromal cell antigen 2 (BST2), fibroblast activation protein (FAP) and tenascin. In an embodiment, the FAP-specific antibody is, competes for binding with, or has the same CDRs as, sibrotuzumab. In embodiments, the MDSC antigen is chosen from one or more of: CD33, CD11b, C₁₄, CD15, and CD66b. Accordingly, in some embodiments, the tumor-supporting antigen is chosen from one or more of: bone marrow stromal cell antigen 2 (BST2), fibroblast activation protein (FAP) or tenascin, CD33, CD11b, C₁₄, CD15, and CD66b.

### Antigen Binding Domain Structures

In some embodiments, the antigen binding domain of the encoded CAR molecule comprises an antibody, an antibody fragment, an scFv, a Fv, a Fab, a (Fab′)₂, a single domain antibody (SDAB), a VH or VL domain, a camelid VHH domain or a bi-functional (e.g. bi-specific) hybrid antibody (e.g., Lanzavecchia et al., Eur. J. Immunol. 17, 105 (1987)).

In some instances, scFvs can be prepared according to method known in the art (see, for example, Bird et al., (1988) Science 242:423-426 and Huston et al., (1988) Proc. Natl. Acad. Sci. USA 85:5879-5883). ScFv molecules can be produced by linking VH and VL regions together using flexible polypeptide linkers. The scFv molecules comprise a linker (e.g., a Ser-Gly linker) with an optimized length and/or amino acid composition. The linker length can greatly affect how the variable regions of a scFv fold and interact. In fact, if a short polypeptide linker is employed (e.g., between 5-10 amino acids) intrachain folding is prevented. Interchain folding is also required to bring the two variable regions together to form a functional epitope binding site. For examples of linker orientation and size see, e.g., Hollinger et al. 1993 Proc Natl Acad. Sci. U.S.A. 90:6444-6448, U.S. Patent Application Publication Nos. 2005/0100543, 2005/0175606, 2007/0014794, and PCT publication Nos. WO2006/020258 and WO2007/024715, is incorporated herein by reference.

An scFv can comprise a linker of at least 1, 2, 3, 4, 5, 6, 7, 8, 9, 10, 11, 12, 13, 14, 15, 16, 17, 18, 19, 20, 25, 30, 35, 40, 45, 50, or more amino acid residues between its VL and VH regions. The linker sequence may comprise any naturally occurring amino acid. In some embodiments, the linker sequence comprises amino acids glycine and serine. In another embodiment, the linker sequence comprises sets of glycine and serine repeats such as (Gly₄Ser)n, where n is a positive integer equal to or greater than 1 (SEQ ID NO: 217). In one embodiment, the linker can be (Gly₄Ser)₄ (SEQ ID NO: 215) or (Gly₄Ser)₃(SEQ ID NO: 216). Variation in the linker length may retain or enhance activity, giving rise to superior efficacy in activity studies.

In another aspect, the antigen binding domain is a T cell receptor (“TCR”), or a fragment thereof, for example, a single chain TCR (scTCR). Methods to make such TCRs are known in the art. See, e.g., Willemsen R A et al, Gene Therapy 7: 1369-1377 (2000); Zhang T et al, Cancer Gene Ther 11: 487-496 (2004); Aggen et al, Gene Ther. 19(4):365-74 (2012) (references are incorporated herein by its entirety). For example, scTCR can be engineered that contains the Vα and Vβ genes from a T cell clone linked by a linker (e.g., a flexible peptide). This approach is very useful to cancer associated target that itself is intracellular, however, a fragment of such antigen (peptide) is presented on the surface of the cancer cells by MHC.

In certain embodiments, the encoded antigen binding domain has a binding affinity KD of 10⁻⁴ M to 10⁻⁸ M.

In one embodiment, the encoded CAR molecule comprises an antigen binding domain that has a binding affinity KD of 10⁻⁴ M to 10⁻⁸ M, e.g., 10⁻⁵ M to 10⁻⁷ M, e.g., 10⁻⁶ M or 10⁻⁷ M, for the target antigen. In one embodiment, the antigen binding domain has a binding affinity that is at least five-fold, 10-fold, 20-fold, 30-fold, 50-fold, 100-fold or 1,000-fold less than a reference antibody, e.g., an antibody described herein. In one embodiment, the encoded antigen binding domain has a binding affinity at least 5-fold less than a reference antibody (e.g., an antibody from which the antigen binding domain is derived). In one aspect such antibody fragments are functional in that they provide a biological response that can include, but is not limited to, activation of an immune response, inhibition of signal-transduction origination from its target antigen, inhibition of kinase activity, and the like, as will be understood by a skilled artisan. In one aspect, the antigen binding domain of the CAR is a scFv antibody fragment that is humanized compared to the murine sequence of the scFv from which it is derived.

In one aspect, the antigen binding domain of a CAR of the disclosure (e.g., a scFv) is encoded by a nucleic acid molecule whose sequence has been codon optimized for expression in a mammalian cell. In one aspect, entire CAR construct of the disclosure is encoded by a nucleic acid molecule whose entire sequence has been codon optimized for expression in a mammalian cell. Codon optimization refers to the discovery that the frequency of occurrence of synonymous codons (i.e., codons that code for the same amino acid) in coding DNA is biased in different species. Such codon degeneracy allows an identical polypeptide to be encoded by a variety of nucleotide sequences. A variety of codon optimization methods is known in the art, and include, e.g., methods disclosed in at least U.S. Pat. Nos. 5,786,464 and 6,114,148.

### Antigen Binding Domains (and the Targeted Antigens)

In one embodiment, an antigen binding domain against CD19 is an antigen binding portion, e.g., CDRs, of a CAR, antibody or antigen-binding fragment thereof described in, e.g., PCT publication WO2012/079000; PCT publication WO2014/153270; Kochenderfer, J. N. et al., J. Immunother. 32 (7), 689-702 (2009); Kochenderfer, J. N., et al., Blood, 116 (20), 4099-4102 (2010); PCT publication WO2014/031687; Bejcek, Cancer Research, 55, 2346-2351, 1995; or U.S. Pat. No. 7,446,190.

In one embodiment, an antigen binding domain against mesothelin is an antigen binding portion, e.g., CDRs, of an antibody, antigen-binding fragment or CAR described in, e.g., PCT publication WO2015/090230. In one embodiment, an antigen binding domain against mesothelin is an antigen binding portion, e.g., CDRs, of an antibody, antigen-binding fragment, or CAR described in, e.g., PCT publication WO1997/025068, WO1999/028471, WO2005/014652, WO2006/099141, WO2009/045957, WO2009/068204, WO2013/142034, WO2013/040557, or WO2013/063419. In one embodiment, an antigen binding domain against mesothelin is an antigen binding portion, e.g., CDRs, of an antibody, antigen-binding fragment, or CAR described in WO/2015/090230.

In one embodiment, an antigen binding domain against CD123 is an antigen binding portion, e.g., CDRs, of an antibody, antigen-binding fragment or CAR described in, e.g., PCT publication WO2014/130635. In one embodiment, an antigen binding domain against CD123 is an antigen binding portion, e.g., CDRs, of an antibody, antigen-binding fragment, or CAR described in, e.g., PCT publication WO2014/138805, WO2014/138819, WO2013/173820, WO2014/144622, WO2001/66139, WO2010/126066, WO2014/144622, or US2009/0252742. In one embodiment, an antigen binding domain against CD123 is an antigen binding portion, e.g., CDRs, of an antibody, antigen-binding fragment, or CAR described in WO/2016/028896.

In one embodiment, an antigen binding domain against EGFRvIII is an antigen binding portion, e.g., CDRs, of an antibody, antigen-binding fragment or CAR described in, e.g., WO/2014/130657.

In one embodiment, an antigen binding domain against CD22 is an antigen binding portion, e.g., CDRs, of an antibody described in, e.g., Haso et al., Blood, 121(7): 1165-1174 (2013); Wayne et al., Clin Cancer Res 16(6): 1894-1903 (2010); Kato et al., Leuk Res 37(1):83-88 (2013); Creative BioMart (creativebiomart.net): MOM-18047-S(P).

In one embodiment, an antigen binding domain against CS-1 is an antigen binding portion, e.g., CDRs, of Elotuzumab (BMS), see e.g., Tai et al., 2008, Blood 112(4):1329-37; Tai et al., 2007, Blood. 110(5):1656-63.

In one embodiment, an antigen binding domain against CLL-1 is an antigen binding portion, e.g., CDRs, of an antibody available from R&D, ebiosciences, Abcam, for example, PE-CLL1-hu Cat #353604 (BioLegend); and PE-CLL1 (CLEC12A) Cat #562566 (BD). In one embodiment, an antigen binding domain against CLL-1 is an antigen binding portion, e.g., CDRs, of an antibody, antigen-binding fragment, or CAR described in WO/2016/014535.

In one embodiment, an antigen binding domain against CD33 is an antigen binding portion, e.g., CDRs, of an antibody described in, e.g., Bross et al., Clin Cancer Res 7(6):1490-1496 (2001) (Gemtuzumab Ozogamicin, hP67.6),Caron et al., Cancer Res 52(24):6761-6767 (1992) (Lintuzumab, HuM195), Lapusan et al., Invest New Drugs 30(3):1121-1131 (2012) (AVE9633), Aigner et al., Leukemia 27(5): 1107-1115 (2013) (AMG330, CD33 BiTE), Dutour et al., Adv hematol 2012:683065 (2012), and Pizzitola et al., Leukemia doi:10.1038/Lue.2014.62 (2014). In one embodiment, an antigen binding domain against CD33 is an antigen binding portion, e.g., CDRs, of an antibody, antigen-binding fragment, or CAR described in WO/2016/014576.

In one embodiment, an antigen binding domain against GD2 is an antigen binding portion, e.g., CDRs, of an antibody described in, e.g., Mujoo et al., Cancer Res. 47(4):1098-1104 (1987); Cheung et al., Cancer Res 45(6):2642-2649 (1985), Cheung et al., J Clin Oncol 5(9):1430-1440 (1987), Cheung et al., J Clin Oncol 16(9):3053-3060 (1998), Handgretinger et al., Cancer Immunol Immunother 35(3):199-204 (1992). In some embodiments, an antigen binding domain against GD2 is an antigen binding portion of an antibody selected from mAb 14.18, 14G2a, ch14.18, hu14.18, 3F8, hu3F8, 3G6, 8B6, 60C3, 10B8, ME36.1, and 8H9, see e.g., WO2012033885, WO2013040371, WO2013192294, WO2013061273, WO2013123061, WO2013074916, and WO201385552. In some embodiments, an antigen binding domain against GD2 is an antigen binding portion of an antibody described in US Publication No.: 20100150910 or PCT Publication No.: WO 2011160119.

In one embodiment, an antigen binding domain against BCMA is an antigen binding portion, e.g., CDRs, of an antibody described in, e.g., WO2012163805, WO200112812, and WO2003062401. In one embodiment, an antigen binding domain against BCMA is an antigen binding portion, e.g., CDRs, of an antibody, antigen-binding fragment, or CAR described in WO/2016/014565.

In one embodiment, an antigen binding domain against Tn antigen is an antigen binding portion, e.g., CDRs, of an antibody described in, e.g., U.S. Pat. No. 8,440,798, Brooks et al., PNAS 107(22):10056-10061 (2010), and Stone et al., OncoImmunology 1(6):863-873(2012).

In one embodiment, an antigen binding domain against PSMA is an antigen binding portion, e.g., CDRs, of an antibody described in, e.g., Parker et al., Protein Expr Purif 89(2):136-145 (2013), US 20110268656 (J591 ScFv); Frigerio et al, European J Cancer 49(9):2223-2232 (2013) (scFvD2B); WO 2006125481 (mAbs 3/A12, 3/E7 and 3/F11) and single chain antibody fragments (scFv A5 and D7).

In one embodiment, an antigen binding domain against ROR1 is an antigen binding portion, e.g., CDRs, of an antibody described in, e.g., Hudecek et al., Clin Cancer Res 19(12):3153-3164 (2013); WO 2011159847; and US20130101607.

In one embodiment, an antigen binding domain against FLT3 is an antigen binding portion, e.g., CDRs, of an antibody described in, e.g., WO2011076922, U.S. Pat. No. 5,777,084, EP0754230, US20090297529, and several commercial catalog antibodies (R&D, ebiosciences, Abcam).

In one embodiment, an antigen binding domain against TAG72 is an antigen binding portion, e.g., CDRs, of an antibody described in, e.g., Hombach et al., Gastroenterology 113(4):1163-1170 (1997); and Abcam ab691.

In one embodiment, an antigen binding domain against FAP is an antigen binding portion, e.g., CDRs, of an antibody described in, e.g., Ostermann et al., Clinical Cancer Research 14:4584-4592 (2008) (FAP5), US Pat. Publication No. 2009/0304718; sibrotuzumab (see e.g., Hofheinz et al., Oncology Research and Treatment 26(1), 2003); and Tran et al., J Exp Med 210(6):1125-1135 (2013).

In one embodiment, an antigen binding domain against CD38 is an antigen binding portion, e.g., CDRs, of daratumumab (see, e.g., Groen et al., Blood 116(21):1261-1262 (2010); MOR202 (see, e.g., U.S. Pat. No. 8,263,746); or antibodies described in U.S. Pat. No. 8,362,211.

In one embodiment, an antigen binding domain against CD44v6 is an antigen binding portion, e.g., CDRs, of an antibody described in, e.g., Casucci et al., Blood 122(20):3461-3472 (2013).

In one embodiment, an antigen binding domain against CEA is an antigen binding portion, e.g., CDRs, of an antibody described in, e.g., Chmielewski et al., Gastoenterology 143(4):1095-1107 (2012).

In one embodiment, an antigen binding domain against EPCAM is an antigen binding portion, e.g., CDRS, of an antibody selected from MT110, EpCAM-CD3 bispecific Ab (see, e.g., clinicaltrials.gov/ct2/show/NCT00635596); Edrecolomab; 3622W94; ING-1; and adecatumumab (MT201).

In one embodiment, an antigen binding domain against PRSS21 is an antigen binding portion, e.g., CDRs, of an antibody described in U.S. Pat. No. 8,080,650.

In one embodiment, an antigen binding domain against B7H3 is an antigen binding portion, e.g., CDRs, of an antibody MGA271 (Macrogenics).

In one embodiment, an antigen binding domain against KIT is an antigen binding portion, e.g., CDRs, of an antibody described in, e.g., U.S. Pat. No. 7,915,391, US20120288506, and several commercial catalog antibodies.

In one embodiment, an antigen binding domain against IL-13Ra2 is an antigen binding portion, e.g., CDRs, of an antibody described in, e.g., WO2008/146911, WO2004087758, several commercial catalog antibodies, and WO2004087758.

In one embodiment, an antigen binding domain against CD30 is an antigen binding portion, e.g., CDRs, of an antibody described in, e.g., U.S. Pat. No. 7,090,843 B1, and EP0805871.

In one embodiment, an antigen binding domain against GD3 is an antigen binding portion, e.g., CDRs, of an antibody described in, e.g., U.S. Pat. Nos. 7,253,263; 8,207,308; US 20120276046; EP1013761; WO2005035577; and U.S. Pat. No. 6,437,098.

In one embodiment, an antigen binding domain against CD171 is an antigen binding portion, e.g., CDRs, of an antibody described in, e.g., Hong et al., J Immunother 37(2):93-104 (2014).

In one embodiment, an antigen binding domain against IL-11Ra is an antigen binding portion, e.g., CDRs, of an antibody available from Abcam (cat #ab55262) or Novus Biologicals (cat #EPR5446). In another embodiment, an antigen binding domain again IL-11Ra is a peptide, see, e.g., Huang et al., Cancer Res 72(1):271-281 (2012).

In one embodiment, an antigen binding domain against PSCA is an antigen binding portion, e.g., CDRs, of an antibody described in, e.g., Morgenroth et al., Prostate 67(10):1121-1131 (2007) (scFv 7F5); Nejatollahi et al., J of Oncology 2013(2013), article ID 839831 (scFv C₅-II); and US Pat Publication No. 20090311181.

In one embodiment, an antigen binding domain against VEGFR2 is an antigen binding portion, e.g., CDRs, of an antibody described in, e.g., Chinnasamy et al., J Clin Invest 120(11):3953-3968 (2010).

In one embodiment, an antigen binding domain against LewisY is an antigen binding portion, e.g., CDRs, of an antibody described in, e.g., Kelly et al., Cancer Biother Radiopharm 23(4):411-423 (2008) (hu3S193 Ab (scFvs)); Dolezal et al., Protein Engineering 16(1):47-56 (2003) (NC10 scFv).

In one embodiment, an antigen binding domain against CD24 is an antigen binding portion, e.g., CDRs, of an antibody described in, e.g., Maliar et al., Gastroenterology 143(5):1375-1384 (2012).

In one embodiment, an antigen binding domain against PDGFR-beta is an antigen binding portion, e.g., CDRs, of an antibody Abcam ab32570.

In one embodiment, an antigen binding domain against SSEA-4 is an antigen binding portion, e.g., CDRs, of antibody MC813 (Cell Signaling), or other commercially available antibodies.

In one embodiment, an antigen binding domain against CD20 is an antigen binding portion, e.g., CDRs, of the antibody Rituximab, Ofatumumab, Ocrelizumab, Veltuzumab, or GA101.

In one embodiment, an antigen binding domain against Folate receptor alpha is an antigen binding portion, e.g., CDRs, of the antibody IMGN853, or an antibody described in US20120009181; U.S. Pat. No. 4,851,332, LK26: U.S. Pat. No. 5,952,484.

In one embodiment, an antigen binding domain against ERBB2 (Her2/neu) is an antigen binding portion, e.g., CDRs, of the antibody trastuzumab, or pertuzumab.

In one embodiment, an antigen binding domain against MUC1 is an antigen binding portion, e.g., CDRs, of the antibody SAR566658.

In one embodiment, the antigen binding domain against EGFR is antigen binding portion, e.g., CDRs, of the antibody cetuximab, panitumumab, zalutumumab, nimotuzumab, or matuzumab.

In one embodiment, an antigen binding domain against NCAM is an antigen binding portion, e.g., CDRs, of the antibody clone 2-2B: MAB5324 (EMD Millipore).

In one embodiment, an antigen binding domain against Ephrin B2 is an antigen binding portion, e.g., CDRs, of an antibody described in, e.g., Abengozar et al., Blood 119(19):4565-4576 (2012).

In one embodiment, an antigen binding domain against IGF-I receptor is an antigen binding portion, e.g., CDRs, of an antibody described in, e.g., U.S. Pat. No. 8,344,112 B2; EP2322550 A1; WO 2006/138315, or PCT/US2006/022995.

In one embodiment, an antigen binding domain against CAIX is an antigen binding portion, e.g., CDRs, of the antibody clone 303123 (R&D Systems).

In one embodiment, an antigen binding domain against LMP2 is an antigen binding portion, e.g., CDRs, of an antibody described in, e.g., U.S. Pat. No. 7,410,640, or US20050129701.

In one embodiment, an antigen binding domain against gp100 is an antigen binding portion, e.g., CDRs, of the antibody HMB45, NKIbetaB, or an antibody described in WO2013165940, or US20130295007 In one embodiment, an antigen binding domain against tyrosinase is an antigen binding portion, e.g., CDRs, of an antibody described in, e.g., U.S. Pat. No. 5,843,674; or U.S. Ser. No. 19/950,504048.

In one embodiment, an antigen binding domain against EphA2 is an antigen binding portion, e.g., CDRs, of an antibody described in, e.g., Yu et al., Mol Ther 22(1):102-111 (2014).

In one embodiment, an antigen binding domain against GD3 is an antigen binding portion, e.g., CDRs, of an antibody described in, e.g., U.S. Pat. Nos. 7,253,263; 8,207,308; US 20120276046; EP1013761 A3; 20120276046; WO2005035577; or U.S. Pat. No. 6,437,098.

In one embodiment, an antigen binding domain against fucosyl GM1 is an antigen binding portion, e.g., CDRs, of an antibody described in, e.g., US20100297138; or WO2007/067992.

In one embodiment, an antigen binding domain against sLe is an antigen binding portion, e.g., CDRs, of the antibody G193 (for lewis Y), see Scott A M et al, Cancer Res 60: 3254-61 (2000), also as described in Neeson et al, J Immunol May 2013 190 (Meeting Abstract Supplement) 177.10.

In one embodiment, an antigen binding domain against GM3 is an antigen binding portion, e.g., CDRs, of the antibody CA 2523449 (mAb 14F7).

In one embodiment, an antigen binding domain against HMWMAA is an antigen binding portion, e.g., CDRs, of an antibody described in, e.g., Kmiecik et al., Oncoimmunology 3(1):e27185 (2014) (PMID: 24575382) (mAb9.2.27); U.S. Pat. No. 6,528,481; WO2010033866; or US 20140004124.

In one embodiment, an antigen binding domain against o-acetyl-GD2 is an antigen binding portion, e.g., CDRs, of the antibody 8B6.

In one embodiment, an antigen binding domain against TEM1/CD248 is an antigen binding portion, e.g., CDRs, of an antibody described in, e.g., Marty et al., Cancer Lett 235(2):298-308 (2006); Zhao et al., J Immunol Methods 363(2):221-232 (2011).

In one embodiment, an antigen binding domain against CLDN6 is an antigen binding portion, e.g., CDRs, of the antibody IMAB027 (Ganymed Pharmaceuticals), see e.g., clinicaltrial.gov/show/NCT02054351.

In one embodiment, an antigen binding domain against TSHR is an antigen binding portion, e.g., CDRs, of an antibody described in, e.g., U.S. Pat. Nos. 8,603,466; 8,501,415; or U.S. Pat. No. 8,309,693.

In one embodiment, an antigen binding domain against GPRC5D is an antigen binding portion, e.g., CDRs, of the antibody FAB6300A (R&D Systems); or LS-A4180 (Lifespan Biosciences).

In one embodiment, an antigen binding domain against CD97 is an antigen binding portion, e.g., CDRs, of an antibody described in, e.g., U.S. Pat. No. 6,846,911; de Groot et al., J Immunol 183(6):4127-4134 (2009); or an antibody from R&D:MAB3734.

In one embodiment, an antigen binding domain against ALK is an antigen binding portion, e.g., CDRs, of an antibody described in, e.g., Mino-Kenudson et al., Clin Cancer Res 16(5):1561-1571 (2010).

In one embodiment, an antigen binding domain against polysialic acid is an antigen binding portion, e.g., CDRs, of an antibody described in, e.g., Nagae et al., J Biol Chem 288(47):33784-33796 (2013).

In one embodiment, an antigen binding domain against PLAC1 is an antigen binding portion, e.g., CDRs, of an antibody described in, e.g., Ghods et al., Biotechnol Appl Biochem 2013 doi:10.1002/bab.1177.

In one embodiment, an antigen binding domain against GloboH is an antigen binding portion of the antibody VK9; or an antibody described in, e.g., Kudryashov V et al, Glycoconj J.15(3):243-9 (1998), Lou et al., Proc Natl Acad Sci USA 111(7):2482-2487 (2014); MBrl: Bremer E-G et al. J Biol Chem 259:14773-14777 (1984).

In one embodiment, an antigen binding domain against NY—BR-1 is an antigen binding portion, e.g., CDRs of an antibody described in, e.g., Jager et al., Appl Immunohistochem Mol Morphol 15(1):77-83 (2007).

In one embodiment, an antigen binding domain against WT-1 is an antigen binding portion, e.g., CDRs, of an antibody described in, e.g., Dao et al., Sci Transl Med 5(176):176ra33 (2013); or WO2012/135854.

In one embodiment, an antigen binding domain against MAGE-A1 is an antigen binding portion, e.g., CDRs, of an antibody described in, e.g., Willemsen et al., J Immunol 174(12):7853-7858 (2005) (TCR-like scFv).

In one embodiment, an antigen binding domain against sperm protein 17 is an antigen binding portion, e.g., CDRs, of an antibody described in, e.g., Song et al., Target Oncol 2013 Aug. 14 (PMID: 23943313); Song et al., Med Oncol 29(4):2923-2931 (2012).

In one embodiment, an antigen binding domain against Tie 2 is an antigen binding portion, e.g., CDRs, of the antibody AB33 (Cell Signaling Technology).

In one embodiment, an antigen binding domain against MAD-CT-2 is an antigen binding portion, e.g., CDRs, of an antibody described in, e.g., PMID: 2450952; U.S. Pat. No. 7,635,753.

In one embodiment, an antigen binding domain against Fos-related antigen 1 is an antigen binding portion, e.g., CDRs, of the antibody 12F9 (Novus Biologicals).

In one embodiment, an antigen binding domain against MelanA/MART1 is an antigen binding portion, e.g., CDRs, of an antibody described in, EP2514766 A2; or U.S. Pat. No. 7,749,719.

In one embodiment, an antigen binding domain against sarcoma translocation breakpoints is an antigen binding portion, e.g., CDRs, of an antibody described in, e.g., Luo et al, EMBO Mol. Med. 4(6):453-461 (2012).

In one embodiment, an antigen binding domain against TRP-2 is an antigen binding portion, e.g., CDRs, of an antibody described in, e.g., Wang et al, J Exp Med. 184(6):2207-16 (1996).

In one embodiment, an antigen binding domain against CYP1B1 is an antigen binding portion, e.g., CDRs, of an antibody described in, e.g., Maecker et al, Blood 102 (9): 3287-3294 (2003).

In one embodiment, an antigen binding domain against RAGE-1 is an antigen binding portion, e.g., CDRs, of the antibody MAB5328 (EMD Millipore).

In one embodiment, an antigen binding domain against human telomerase reverse transcriptase is an antigen binding portion, e.g., CDRs, of the antibody cat no: LS-B95-100 (Lifespan Biosciences)

In one embodiment, an antigen binding domain against intestinal carboxyl esterase is an antigen binding portion, e.g., CDRs, of the antibody 4F12: cat no: LS-B6190-50 (Lifespan Biosciences).

In one embodiment, an antigen binding domain against mut hsp70-2 is an antigen binding portion, e.g., CDRs, of the antibody Lifespan Biosciences: monoclonal: cat no: LS-C133261-100 (Lifespan Biosciences).

In one embodiment, an antigen binding domain against CD79a is an antigen binding portion, e.g., CDRs, of the antibody Anti-CD79a antibody [HM47/A9](ab3121), available from Abcam; antibody CD79A Antibody #3351 available from Cell Signaling Technology; or antibody HPA017748-Anti-CD79A antibody produced in rabbit, available from Sigma Aldrich.

In one embodiment, an antigen binding domain against CD79b is an antigen binding portion, e.g., CDRs, of the antibody polatuzumab vedotin, anti-CD79b described in Doman et al., “Therapeutic potential of an anti-CD79b antibody-drug conjugate, anti-CD79b-vc-MMAE, for the treatment of non-Hodgkin lymphoma” Blood. 2009 Sep. 24; 114(13):2721-9. doi: 10.1182/blood-2009-02-205500. Epub 2009 Jul. 24, or the bispecific antibody Anti-CD79b/CD3 described in “4507 Pre-Clinical Characterization of T Cell-Dependent Bispecific Antibody Anti-CD79b/CD3 As a Potential Therapy for B Cell Malignancies” Abstracts of 56^{th} ASH Annual Meeting and Exposition, San Francisco, CA December 6-9 2014.

In one embodiment, an antigen binding domain against CD72 is an antigen binding portion, e.g., CDRs, of the antibody J3-109 described in Myers, and Uckun, “An anti-CD72 immunotoxin against therapy-refractory B-lineage acute lymphoblastic leukemia.” Leuk Lymphoma. 1995 June; 18(1-2):119-22, or anti-CD72 (10D6.8.1, mIgG1) described in Polson et al., “Antibody-Drug Conjugates for the Treatment of Non-Hodgkin's Lymphoma: Target and Linker-Drug Selection” Cancer Res Mar. 15, 2009 69; 2358.

In one embodiment, an antigen binding domain against LAIR1 is an antigen binding portion, e.g., CDRs, of the antibody ANT-301 LAIR1 antibody, available from ProSpec; or anti-human CD305 (LAIR1) Antibody, available from BioLegend.

In one embodiment, an antigen binding domain against FCAR is an antigen binding portion, e.g., CDRs, of the antibody CD89/FCARAntibody (Catalog #10414-HO8H), available from Sino Biological Inc.

In one embodiment, an antigen binding domain against LILRA2 is an antigen binding portion, e.g., CDRs, of the antibody LILRA2 monoclonal antibody (M17), clone 3C₇, available from Abnova, or Mouse Anti-LILRA2 antibody, Monoclonal (2D7), available from Lifespan Biosciences.

In one embodiment, an antigen binding domain against CD300LF is an antigen binding portion, e.g., CDRs, of the antibody Mouse Anti-CMRF35-like molecule 1 antibody, Monoclonal[UP-D2], available from BioLegend, or Rat Anti-CMRF35-like molecule 1 antibody, Monoclonal[234903], available from R&D Systems.

In one embodiment, an antigen binding domain against CLEC12A is an antigen binding portion, e.g., CDRs, of the antibody Bispecific T cell Engager (BiTE) scFv-antibody and ADC described in Noordhuis et al., “Targeting of CLEC12A In Acute Myeloid Leukemia by Antibody-Drug-Conjugates and Bispecific CLL-1×CD3 BiTE Antibody” 53^{rd} ASH Annual Meeting and Exposition, December 10-13, 2011, and MCLA-117 (Merus).

In one embodiment, an antigen binding domain against BST2 (also called CD317) is an antigen binding portion, e.g., CDRs, of the antibody Mouse Anti-CD317 antibody, Monoclonal[3H4], available from Antibodies-Online or Mouse Anti-CD317 antibody, Monoclonal[696739], available from R&D Systems.

In one embodiment, an antigen binding domain against EMR2 (also called CD312) is an antigen binding portion, e.g., CDRs, of the antibody Mouse Anti-CD312 antibody, Monoclonal[LS-B8033] available from Lifespan Biosciences, or Mouse Anti-CD312 antibody, Monoclonal[494025] available from R&D Systems.

In one embodiment, an antigen binding domain against LY75 is an antigen binding portion, e.g., CDRs, of the antibody Mouse Anti-Lymphocyte antigen 75 antibody, Monoclonal[HD30] available from EMD Millipore or Mouse Anti-Lymphocyte antigen 75 antibody, Monoclonal[A15797] available from Life Technologies.

In one embodiment, an antigen binding domain against GPC3 is an antigen binding portion, e.g., CDRs, of the antibody hGC33 described in Nakano K, Ishiguro T, Konishi H, et al. Generation of a humanized anti-glypican 3 antibody by CDR grafting and stability optimization. Anticancer Drugs. 2010 November; 21(10):907-916, or MDX-1414, HN3, or YP7, all three of which are described in Feng et al., “Glypican-3 antibodies: a new therapeutic target for liver cancer.” FEBS Lett. 2014 Jan. 21; 588(2):377-82.

In one embodiment, an antigen binding domain against FCRL5 is an antigen binding portion, e.g., CDRs, of the anti-FcRL5 antibody described in Elkins et al., “FcRL5 as a target of antibody-drug conjugates for the treatment of multiple myeloma” Mol Cancer Ther. 2012 October; 11(10):2222-32. In one embodiment, an antigen binding domain against FCRL5 is an antigen binding portion, e.g., CDRs, of the anti-FcRL5 antibody described in, for example, WO2001/038490, WO/2005/117986, WO2006/039238, WO2006/076691, WO2010/114940, WO2010/120561, or WO2014/210064.

In one embodiment, an antigen binding domain against IGLL1 is an antigen binding portion, e.g., CDRs, of the antibody Mouse Anti-Immunoglobulin lambda-like polypeptide 1 antibody, Monoclonal[AT1G4] available from Lifespan Biosciences, Mouse Anti-Immunoglobulin lambda-like polypeptide 1 antibody, Monoclonal[HSL11] available from BioLegend.

In one embodiment, the antigen binding domain comprises one, two three (e.g., all three) heavy chain CDRs, HC CDR1, HC CDR2 and HC CDR3, from an antibody listed above, and/or one, two, three (e.g., all three) light chain CDRs, LC CDR1, LC CDR2 and LC CDR3, from an antibody listed above. In one embodiment, the antigen binding domain comprises a heavy chain variable region and/or a variable light chain region of an antibody listed above.

In another aspect, the antigen binding domain comprises a humanized antibody or an antibody fragment. In some aspects, a non-human antibody is humanized, where specific sequences or regions of the antibody are modified to increase similarity to an antibody naturally produced in a human or fragment thereof. In one aspect, the antigen binding domain is humanized.

In an embodiment, the antigen-binding domain of a CAR, e.g., a CAR expressed by a cell of the disclosure, binds to CD19. CD19 is found on B cells throughout differentiation of the lineage from the pro/pre-B cell stage through the terminally differentiated plasma cell stage. In an embodiment, the antigen binding domain is a murine scFv domain that binds to human CD19, e.g., the antigen binding domain of CTL019 (e.g., SEQ ID NO: 218). In an embodiment, the antigen binding domain is a humanized antibody or antibody fragment, e.g., scFv domain, derived from the murine CTL019 scFv. In an embodiment, the antigen binding domain is a human antibody or antibody fragment that binds to human CD19. Exemplary scFv domains (and their sequences, e.g., CDRs, VL and VH sequences) that bind to CD19 are provided in Table 12a. The scFv domain sequences provided in Table 12a include a light chain variable region (VL) and a heavy chain variable region (VH). The VL and VH are attached by a linker comprising the sequence GGGGSGGGGSGGGGS (SEQ ID NO: 216), e.g., in the following orientation: VL-linker-VH.

TABLE 12a
Antigen Binding domains that bind CD 19

| | | | SEQ |
|---|---|---|---|
| | | Amino Acid | ID |
| Antigen | Name | Sequence | NO: |
| CD19 | muCTL019 | DIQMTQTTSS | 218 |
| | | LSASLGDRVT | |
| | | ISCRASQDIS | |
| | | KYLNWYQQKP | |
| | | DGTVKLLIYH | |
| | | TSRLHSGVPS | |
| | | RFSGSGSGTD | |
| | | YSLTISNLEQ | |
| | | EDIATYFCQQ | |
| | | GNTLPYTFGG | |
| | | GTKLEITGGG | |
| | | GSGGGGSGGG | |
| | | GSEVKLQESG | |
| | | PGLVAPSQSL | |
| | | SVTCTVSGVS | |
| | | LPDYGVSWIR | |
| | | QPPRKGLEWL | |
| | | GVIWGSETTY | |
| | | YNSALKSRLT | |
| | | IIKDNSKSQV | |
| | | FLKMNSLQTD | |
| | | DTAIYYCAKH | |
| | | YYYGGSYAMD | |
| | | YWGQGTSVTV | |
| | | SS | |
| CD19 | huscFv1 | EIVMTQSPAT | 219 |
| | | LSLSPGERAT | |
| | | LSCRASQDIS | |
| | | KYLNWYQQKP | |
| | | GQAPRLLIYH | |
| | | TSRLHSGIPA | |
| | | RFSGSGSGTD | |
| | | YTLTISSLQP | |
| | | EDFAVYFCQQ | |
| | | GNTLPYTFGQ | |
| | | GTKLEIKGGG | |
| | | GSGGGGSGGG | |
| | | GSQVQLQESG | |
| | | PGLVKPSETL | |
| | | SLTCTVSGVS | |
| | | LPDYGVSWIR | |
| | | QPPGKGLEWI | |
| | | GVIWGSETTY | |
| | | YSSSLKSRVT | |
| | | ISKDNSKNQV | |
| | | SLKLSSVTAA | |
| | | DTAVYYCAKH | |
| | | YYYGGSYAMD | |
| | | YWGQGTLVTV | |
| | | SS | |
| CD19 | huscFv2 | EIVMTQSPAT | 220 |
| | | LSLSPGERAT | |
| | | LSCRASQDIS | |
| | | KYLNWYQQKP | |
| | | GQAPRLLIYH | |
| | | TSRLHSGIPA | |
| | | RFSGSGSGTD | |
| | | YTLTISSLQP | |
| | | EDFAVYFCQQ | |
| | | GNTLPYTFGQ | |
| | | GTKLEIKGGG | |
| | | GSGGGGSGGG | |
| | | GSQVQLQESG | |
| | | PGLVKPSETL | |
| | | SLTCTVSGVS | |
| | | LPDYGVSWIR | |
| | | QPPGKGLEWI | |
| | | GVIWGSETTY | |
| | | YQSSLKSRVT | |
| | | ISKDNSKNQV | |
| | | SLKLSSVTAA | |
| | | DTAVYYCAKH | |
| | | YYYGGSYAMD | |
| | | YWGQGTLVTV | |
| | | SS | |
| CD19 | huscFv3 | QVQLQESGPG | 221 |
| | | LVKPSETLSL | |
| | | TCTVSGVSLP | |
| | | DYGVSWIRQP | |
| | | PGKGLEWIGV | |
| | | IWGSETTYYS | |
| | | SSLKSRVTIS | |
| | | KDNSKNQVSL | |
| | | KLSSVTAADT | |
| | | AVYYCAKHYY | |
| | | YGGSYAMDYW | |
| | | GQGTLVTVSS | |
| | | GGGGSGGGGS | |
| | | GGGGSEIVMT | |
| | | QSPATLSLSP | |
| | | GERATLSCRA | |
| | | SQDISKYLNW | |
| | | YQQKPGQAPR | |
| | | LLIYHTSRLH | |
| | | SGIPARFSGS | |
| | | GSGTDYTLTI | |
| | | SSLQPEDFAV | |
| | | YFCQQGNTLP | |
| | | YTFGQGTKLE | |
| | | IK | |
| CD19 | huscFv4 | QVQLQESGPG | 222 |
| | | LVKPSETLSL | |
| | | TCTVSGVSLP | |
| | | DYGVSWIRQP | |
| | | PGKGLEWIGV | |
| | | IWGSETTYYQ | |
| | | SSLKSRVTIS | |
| | | KDNSKNQVSL | |
| | | KLSSVTAADT | |
| | | AVYYCAKHYY | |
| | | YGGSYAMDYW | |
| | | GQGTLVTVSS | |
| | | GGGGSGGGGS | |
| | | GGGGSEIVMT | |
| | | QSPATLSLSP | |
| | | GERATLSCRA | |
| | | SQDISKYLNW | |
| | | YQQKPGQAPR | |
| | | LLIYHTSRLH | |
| | | SGIPARFSGS | |
| | | GSGTDYTLTI | |
| | | SSLQPEDFAV | |
| | | YFCQQGNTLP | |
| | | YTFGQGTKLE | |
| | | IK | |
| CD19 | huscFv5 | EIVMTQSPAT | 223 |
| | | LSLSPGERAT | |
| | | LSCRASQDIS | |
| | | KYLNWYQQKP | |
| | | GQAPRLLIYH | |
| | | TSRLHSGIPA | |
| | | RFSGSGSGTD | |
| | | YTLTISSLQP | |
| | | EDFAVYFCQQ | |
| | | GNTLPYTFGQ | |
| | | GTKLEIKGGG | |
| | | GSGGGGSGGG | |
| | | GSGGGGSQVQ | |
| | | LQESGPGLVK | |
| | | PSETLSLTCT | |
| | | VSGVSLPDYG | |
| | | VSWIRQPPGK | |
| | | GLEWIGVIWG | |
| | | SETTYYSSSL | |
| | | KSRVTISKDN | |
| | | SKNQVSLKLS | |
| | | SVTAADTAVY | |
| | | YCAKHYYYGG | |
| | | SYAMDYWGQG | |
| | | TLVTVSS | |
| CD19 | huscFv6 | EIVMTQSPAT | 224 |
| | | LSLSPGERAT | |
| | | LSCRASQDIS | |
| | | KYLNWYQQKP | |
| | | GQAPRLLIYH | |
| | | TSRLHSGIPA | |
| | | RFSGSGSGTD | |
| | | YTLTISSLQP | |
| | | EDFAVYFCQQ | |
| | | GNTLPYTFGQ | |
| | | GTKLEIKGGG | |
| | | GSGGGGSGGG | |
| | | GSGGGGSQVQ | |
| | | LQESGPGLVK | |
| | | PSETLSLTCT | |
| | | VSGVSLPDYG | |
| | | VSWIRQPPGK | |
| | | GLEWIGVIWG | |
| | | SETTYYQSSL | |
| | | KSRVTISKDN | |
| | | SKNQVSLKLS | |
| | | SVTAADTAVY | |
| | | YCAKHYYYGG | |
| | | SYAMDYWGQG | |
| | | TLVTVSS | |
| CD19 | huscFv7 | QVQLQESGPG | 225 |
| | | LVKPSETLSL | |
| | | TCTVSGVSLP | |
| | | DYGVSWIRQP | |
| | | PGKGLEWIGV | |
| | | IWGSETTYYS | |
| | | SSLKSRVTIS | |
| | | KDNSKNQVSL | |
| | | KLSSVTAADT | |
| | | AVYYCAKHYY | |
| | | YGGSYAMDYW | |
| | | GQGTLVTVSS | |
| | | GGGGSGGGGS | |
| | | GGGGSGGGGS | |
| | | EIVMTQSPAT | |
| | | LSLSPGERAT | |
| | | LSCRASQDIS | |
| | | KYLNWYQQKP | |
| | | GQAPRLLIYH | |
| | | TSRLHSGIPA | |
| | | RFSGSGSGTD | |
| | | YTLTISSLQP | |
| | | EDFAVYFCQQ | |
| | | GNTLPYTFGQ | |
| | | GTKLEIK | |
| CD19 | huscFv8 | QVQLQESGPG | 226 |
| | | LVKPSETLSL | |
| | | TCTVSGVSLP | |
| | | DYGVSWIRQP | |
| | | PGKGLEWIGV | |
| | | IWGSETTYYQ | |
| | | SSLKSRVTIS | |
| | | KDNSKNQVSL | |
| | | KLSSVTAADT | |
| | | AVYYCAKHYY | |
| | | YGGSYAMDYW | |
| | | GQGTLVTVSS | |
| | | GGGGSGGGGS | |
| | | GGGGSGGGGS | |
| | | EIVMTQSPAT | |
| | | LSLSPGERAT | |
| | | LSCRASQDIS | |
| | | KYLNWYQQKP | |
| | | GQAPRLLIYH | |
| | | TSRLHSGIPA | |
| | | RFSGSGSGTD | |
| | | YTLTISSLQP | |
| | | EDFAVYFCQQ | |
| | | GNTLPYTFGQ | |
| | | GTKLEIK | |
| CD19 | huscFv9 | EIVMTQSPAT | 227 |
| | | LSLSPGERAT | |
| | | LSCRASQDIS | |
| | | KYLNWYQQKP | |
| | | GQAPRLLIYH | |
| | | TSRLHSGIPA | |
| | | RFSGSGSGTD | |
| | | YTLTISSLQP | |
| | | EDFAVYFCQQ | |
| | | GNTLPYTFGQ | |
| | | GTKLEIKGGG | |
| | | GSGGGGSGGG | |
| | | GSGGGGSQVQ | |
| | | LQESGPGLVK | |
| | | PSETLSLTCT | |
| | | VSGVSLPDYG | |
| | | VSWIRQPPGK | |
| | | GLEWIGVIWG | |
| | | SETTYYNSSL | |
| | | KSRVTISKDN | |
| | | SKNQVSLKLS | |
| | | SVTAADTAVY | |
| | | YCAKHYYYGG | |
| | | SYAMDYWGQG | |
| | | TLVTVSS | |
| CD19 | Hu | QVQLQESGPG | 228 |
| | ScFv10 | LVKPSETLSL | |
| | | TCTVSGVSLP | |
| | | DYGVSWIRQP | |
| | | PGKGLEWIGV | |
| | | IWGSETTYYN | |
| | | SSLKSRVTIS | |
| | | KDNSKNQVSL | |
| | | KLSSVTAADT | |
| | | AVYYCAKHYY | |
| | | YGGSYAMDYW | |
| | | GQGTLVTVSS | |
| | | GGGGSGGGGS | |
| | | GGGGSGGGGS | |
| | | EIVMTQSPAT | |
| | | LSLSPGERAT | |
| | | LSCRASQDIS | |
| | | KYLNWYQQKP | |
| | | GQAPRLLIYH | |
| | | TSRLHSGIPA | |
| | | RFSGSGSGTD | |
| | | YTLTISSLQP | |
| | | EDFAVYFCQQ | |
| | | GNTLPYTFGQ | |
| | | GTKLEIK | |
| CD19 | Hu | EIVMTQSPAT | 229 |
| | ScFv11 | LSLSPGERAT | |
| | | LSCRASQDIS | |
| | | KYLNWYQQKP | |
| | | GQAPRLLIYH | |
| | | TSRLHSGIPA | |
| | | RFSGSGSGTD | |
| | | YTLTISSLQP | |
| | | EDFAVYFCQQ | |
| | | GNTLPYTFGQ | |
| | | GTKLEIKGGG | |
| | | GSGGGGSGGG | |
| | | GSQVQLQESG | |
| | | PGLVKPSETL | |
| | | SLTCTVSGVS | |
| | | LPDYGVSWIR | |
| | | QPPGKGLEWI | |
| | | GVIWGSETTY | |
| | | YNSSLKSRVT | |
| | | ISKDNSKNQV | |
| | | SLKLSSVTAA | |
| | | DTAVYYCAKH | |
| | | YYYGGSYAMD | |
| | | YWGQGTLVTV | |
| | | SS | |
| CD19 | Hu | QVQLQESGPG | 230 |
| | scFv12 | LVKPSETLSL | |
| | | TCTVSGVSLP | |
| | | DYGVSWIRQP | |
| | | PGKGLEWIGV | |
| | | IWGSETTYYN | |
| | | SSLKSRVTIS | |
| | | KDNSKNQVSL | |
| | | KLSSVTAADT | |
| | | AVYYCAKHYY | |
| | | YGGSYAMDYW | |
| | | GQGTLVTVSS | |
| | | GGGGSGGGGS | |
| | | GGGGSEIVMT | |
| | | QSPATLSLSP | |
| | | GERATLSCRA | |
| | | SQDISKYLNW | |
| | | YQQKPGQAPR | |
| | | LLIYHTSRLH | |
| | | SGIPARFSGS | |
| | | GSGTDYTLTI | |
| | | SSLQPEDFAV | |
| | | YFCQQGNTLP | |
| | | YTFGQGTKLE | |
| | | IK | |

The sequences of the CDR sequences of the scFv domains of the CD 19 antigen binding domains provided in Table 12a are shown in Table 12b for the heavy chain variable domains and in Table 12c for the light chain variable domains. “ID” stands for the respective SEQ ID NO for each CDR.

TABLE 12b
Heavy Chain Variable Domain CDRs

| Description | FW | HCDR1 | ID | HCDR2 | ID | HCDR3 | ID |
|---|---|---|---|---|---|---|---|
| murine_ | | GVSLP | 306 | VIWGSETTY | 307 | HYYYGG | 231 |
| CART19 | | DYGVS | | YNSALKS | | SYAMDY | |
| humanized_ | VH4 | GVSLP | 306 | VIWGSETTY | 308 | HYYYGG | 231 |
| CART19 a | | DYGVS | | YSSSLKS | | SYAMDY | |
| humanized_ | | GVSLP | 306 | VIWGSETTY | 309 | HYYYGG | 231 |
| CART19 b | VH4 | DYGVS | | YQSSLKS | | SYAMDY | |
| humanized_ | VH4 | GVSLP | 306 | VIWGSETTY | 310 | HYYYGG | 231 |
| CART19 c | | DYGVS | | YNSSLKS | | SYAMDY | |

TABLE 12c
Light Chain Variable Domain CDRs

| Description | FW | LCDR1 | ID | LCDR2 | ID | LCDR3 | ID |
|---|---|---|---|---|---|---|---|
| murine_ | | RASQDI | 311 | HTSR | 312 | QQGNT | 232 |
| CART19 | | SKYLN | | LHS | | LPYT | |
| humanized_ | VK3 | RASQDI | 311 | HTSR | 312 | QQGNT | 232 |
| CART19 a | | SKYLN | | LHS | | LPYT | |
| humanized_ | VK3 | RASQDI | 311 | HTSR | 312 | QQGNT | 232 |
| CART19 b | | SKYLN | | LHS | | LPYT | |
| humanized_ | VK3 | RASQDI | 311 | HTSR | 312 | QQGNT | 232 |
| CART19 c | | SKYLN | | LHS | | LPYT | |

In an embodiment, the antigen binding domain comprises an anti-CD19 antibody, or fragment thereof, e.g., a scFv. For example, the antigen binding domain comprises a variable heavy chain and a variable light chain listed in Table 12d. The linker sequence joining the variable heavy and variable light chains can be any of the linker sequences described herein, or alternatively, can be GSTSGSGKPGSGEGSTKG (SEQ ID NO: 233). The light chain variable region and heavy chain variable region of a scFv can be, e.g., in any of the following orientations: light chain variable region-linker-heavy chain variable region or heavy chain variable region-linker-light chain variable region.

TABLE 12d.
Additional Anti-CD19 antibody
binding domains

| Ab | | |
|---|---|---|
| Name | VH Sequence | VL Sequence |
| SJ25-C1 | QVQLLESGAELV | ELVLTQSPKFM |
| | RPGSSVKISCKA | STSVGDRVSVT |
| | SGYAFSSYWMNW | CKASQNVGTNV |
| | VKQRPGQGLEWI | AWYQQKPGQSP |
| | GQIYPGDGDTNY | KPLIYSATYRN |
| | NGKFKGQATLTA | SGVPDRFTGSG |
| | DKSSSTAYMQLS | SGTDFTLTITN |
| | GLTSEDSAVYSC | VQSKDLADYFY |
| | ARKTISSVVDFY | FCQYNRYPYTS |
| | FDYWGQGTTVT | GGGTKLEIKRR |
| | (SEQ ID | S (SEQ ID |
| | NO: 234) | NO: 235) |

| | |
|---|---|
| | ScFv Sequence |
| SJ25-C1 | QVQLLESGAELVRPGSSVKISCKA |
| scFv | SGYAFSSYWMNWVKQRPGQGLEWI |
| | GQIYPGDGDTNYNGKFKGQATLTA |
| | DKSSSTAYMQLSGLTSEDSAVYSC |
| | ARKTISSVVDFYFDYWGQGTTVTG |
| | STSGSGKPGSGEGSTKGELVLTQS |
| | PKFMSTSVGDRVSVTCKASQNVGT |
| | NVAWYQQKPGQSPKPLIYSATYRN |
| | SGVPDRFTGSGSGTDFTLTITNVQ |
| | SKDLADYFYFCQYNRYPYTSGGGT |
| | KLEIKRRS |
| | (SEQ ID NO: 236) |

In one embodiment, the CD19 binding domain comprises one or more (e.g., all three) light chain complementary determining region 1 (LC CDR1), light chain complementary determining region 2 (LC CDR2), and light chain complementary determining region 3 (LC CDR3) of a CD19 binding domain described herein, e.g., provided in Table 12a or 15, and/or one or more (e.g., all three) heavy chain complementary determining region 1 (HC CDR1), heavy chain complementary determining region 2 (HC CDR2), and heavy chain complementary determining region 3 (HC CDR3) of a CD19 binding domain described herein, e.g., provided in Table 12a or 16. In one embodiment, the CD19 binding domain comprises one, two, or all of LC CDR1, LC CDR2, and LC CDR3 of any amino acid sequences as provided in Table 12c, incorporated herein by reference; and one, two or all of HC CDR1, HC CDR2, and HC CDR3 of any amino acid sequences as provided in Table 12b.

Any known CD19 CAR, e.g., the CD19 antigen binding domain of any known CD19 CAR, in the art can be used in accordance with the instant disclosure to construct a CAR. For example, LG-740; CD19 CAR described in the U.S. Pat. Nos. 8,399,645; 7,446,190; Xu et al., Leuk Lymphoma. 2013 54(2):255-260(2012); Cruz et al., Blood 122(17):2965-2973 (2013); Brentjens et al., Blood, 118(18):4817-4828 (2011); Kochenderfer et al., Blood 116(20):4099-102 (2010); Kochenderfer et al., Blood 122 (25):4129-39(2013); and 16th Annu Meet Am Soc Gen Cell Ther (ASGCT) (May 15-18, Salt Lake City) 2013, Abst 10. In one embodiment, an antigen binding domain against CD19 is an antigen binding portion, e.g., CDRs, of a CAR, antibody or antigen-binding fragment thereof described in, e.g., PCT publication WO2012/079000; PCT publication WO2014/153270; Kochenderfer, J. N. et al., J. Immunother. 32 (7), 689-702 (2009); Kochenderfer, J. N., et al., Blood, 116 (20), 4099-4102 (2010); PCT publication WO2014/031687; Bejcek, Cancer Research, 55, 2346-2351, 1995; or U.S. Pat. No. 7,446,190.

In an embodiment, the antigen-binding domain of CAR, e.g., a CAR expressed by a cell of the disclosure, binds to BCMA. BCMA is found preferentially expressed in mature B lymphocytes. In an embodiment, the antigen binding domain is a murine scFv domain that binds to human BCMA. In an embodiment, the antigen binding domain is a humanized antibody or antibody fragment, e.g., scFv domain that binds human BCMA. In an embodiment, the antigen binding domain is a human antibody or antibody fragment that binds to human BCMA. In embodiments, exemplary BCMA CAR constructs are generated using the VH and VL sequences from PCT Publication W02012/0163805 (the contents of which are hereby incorporated by reference in its entirety). In embodiments, additional exemplary BCMA CAR constructs are generated using the VH and VL sequences from PCT Publication WO2016/014565 (the contents of which are hereby incorporated by reference in its entirety). In embodiments, additional exemplary BCMA CAR constructs are generated using the VH and VL sequences from PCT Publication WO2014/122144 (the contents of which are hereby incorporated by reference in its entirety). In embodiments, additional exemplary BCMA CAR constructs are generated using the CAR molecules, and/or the VH and VL sequences from PCT Publication WO2016/014789 (the contents of which are hereby incorporated by reference in its entirety). In embodiments, additional exemplary BCMA CAR constructs are generated using the CAR molecules, and/or the VH and VL sequences from PCT Publication WO2014/089335 (the contents of which are hereby incorporated by reference in its entirety). In embodiments, additional exemplary BCMA CAR constructs are generated using the CAR molecules, and/or the VH and VL sequences from PCT Publication WO2014/140248 (the contents of which are hereby incorporated by reference in its entirety).

Any known BCMA CAR, e.g., the BMCA antigen binding domain of any known BCMA CAR, in the art can be used in accordance with the instant disclosure. For example, those described herein.

### Exemplary CAR Molecules

In one aspect, a CAR, e.g., a CAR expressed by the cell of the disclosure, comprises a CAR molecule comprising an antigen binding domain that binds to a B cell antigen, e.g., as described herein, such as CD19 or BCMA.

In one embodiment, the CAR comprises a CAR molecule comprising a CD19 antigen binding domain (e.g., a murine, human or humanized antibody or antibody fragment that specifically binds to CD19), a transmembrane domain, and an intracellular signaling domain (e.g., an intracellular signaling domain comprising a costimulatory domain and/or a primary signaling domain).

Exemplary CAR molecules described herein are provided in Table 12e. The CAR molecules in Table 12e comprise a CD19 antigen binding domain, e.g., an amino acid sequence of any CD19 antigen binding domain provided in Table 12a.

TABLE 12e
Exemplary CD19 CAR molecules

| | | | SEQ |
|---|---|---|---|
| | | | ID |
| Antigen | Name | Amino Acid Sequence | NO: |
| CD19 | CTL019 | MALPVTALLLPLALLLHAAR | 237 |
| | | PDIQMTQTTSSLSASLGDRV | |
| | | TISCRASQDISKYLNWYQQK | |
| | | PDGTVKLLIYHTSRLHSGVP | |
| | | SRFSGSGSGTDYSLTISNLE | |
| | | QEDIATYFCQQGNTLPYTFG | |
| | | GGTKLEITGGGGSGGGGSGG | |
| | | GGSEVKLQESGPGLVAPSQS | |
| | | LSVTCTVSGVSLPDYGVSWI | |
| | | RQPPRKGLEWLGVIWGSETT | |
| | | YYNSALKSRLTIIKDNSKSQ | |
| | | VFLKMNSLQTDDTAIYYCAK | |
| | | HYYYGGSYAMDYWGQGTSVT | |
| | | VSSTTTPAPRPPTPAPTIAS | |
| | | QPLSLRPEACRPAAGGAVHT | |
| | | RGLDFACDIYIWAPLAGTCG | |
| | | VLLLSLVTTLYCKRGRKKLL | |
| | | YIFKQPFMRPVQTTQEEDGC | |
| | | SCRFPEEEEGGCELRVKFSR | |
| | | SADAPAYKQGQNQLYNELNL | |
| | | GRREEYDVLDKRRGRDPEMG | |
| | | GKPRRKNPQEGLYNELQKDK | |
| | | MAEAYSEIGMKGERRRGKGH | |
| | | DGLYQGLSTATKDTYDALHM | |
| | | QALPPR | |
| CD19 | CAR 1 | MALPVTALLLPLALLLHAAR | 238 |
| | | PEIVMTQSPATLSLSPGERA | |
| | | TLSCRASQDISKYLNWYQQK | |
| | | PGQAPRLLIYHTSRLHSGIP | |
| | | ARFSGSGSGTDYTLTISSLQ | |
| | | PEDFAVYFCQQGNTLPYTFG | |
| | | QGTKLEIKGGGGSGGGGSGG | |
| | | GGSQVQLQESGPGLVKPSET | |
| | | LSLTCTVSGVSLPDYGVSWI | |
| | | RQPPGKGLEWIGVIWGSETT | |
| | | YYSSSLKSRVTISKDNSKNQ | |
| | | VSLKLSSVTAADTAVYYCAK | |
| | | HYYYGGSYAMDYWGQGTLVT | |
| | | VSSTTTPAPRPPTPAPTIAS | |
| | | QPLSLRPEACRPAAGGAVHT | |
| | | RGLDFACDIYIWAPLAGTCG | |
| | | VLLLSLVITLYCKRGRKKLL | |
| | | YIFKQPFMRPVQTTQEEDGC | |
| | | SCRFPEEEEGGCELRVKFSR | |
| | | SADAPAYKQGQNQLYNELNL | |
| | | GRREEYDVLDKRRGRDPEMG | |
| | | GKPRRKNPQEGLYNELQKDK | |
| | | MAEAYSEIGMKGERRRGKGH | |
| | | DGLYQGLSTATKDTYDALHM | |
| | | QALPPR | |
| CD19 | CAR 2 | MALPVTALLLPLALLLHAAR | 239 |
| | | PEIVMTQSPATLSLSPGERA | |
| | | TLSCRASQDISKYLNWYQQK | |
| | | PGQAPRLLIYHTSRLHSGIP | |
| | | ARFSGSGSGTDYTLTISSLQ | |
| | | PEDFAVYFCQQGNTLPYTFG | |
| | | QGTKLEIKGGGGSGGGGSGG | |
| | | GGSQVQLQESGPGLVKPSET | |
| | | LSLTCTVSGVSLPDYGVSWI | |
| | | RQPPGKGLEWIGVIWGSETT | |
| | | YYQSSLKSRVTISKDNSKNQ | |
| | | VSLKLSSVTAADTAVYYCAK | |
| | | HYYYGGSYAMDYWGQGTLVT | |
| | | VSSTTTPAPRPPTPAPTIAS | |
| | | QPLSLRPEACRPAAGGAVHT | |
| | | RGLDFACDIYIWAPLAGTCG | |
| | | VLLLSLVITLYCKRGRKKLL | |
| | | YIFKQPFMRPVQTTQEEDGC | |
| | | SCRFPEEEEGGCELRVKFSR | |
| | | SADAPAYKQGQNQLYNELNL | |
| | | GRREEYDVLDKRRGRDPEMG | |
| | | GKPRRKNPQEGLYNELQKDK | |
| | | MAEAYSEIGMKGERRRGKGH | |
| | | DGLYQGLSTATKDTYDALHM | |
| | | QALPPR | |
| CD19 | CAR 3 | MALPVTALLLPLALLLHAAR | 240 |
| | | PQVQLQESGPGLVKPSETLS | |
| | | LTCTVSGVSLPDYGVSWIRQ | |
| | | PPGKGLEWIGVIWGSETTYY | |
| | | SSSLKSRVTISKDNSKNQVS | |
| | | LKLSSVTAADTAVYYCAKHY | |
| | | YYGGSYAMDYWGQGTLVTVS | |
| | | SGGGGSGGGGSGGGGSEIVM | |
| | | TQSPATLSLSPGERATLSCR | |
| | | ASQDISKYLNWYQQKPGQAP | |
| | | RLLIYHTSRLHSGIPARFSG | |
| | | SGSGTDYTLTISSLQPEDFA | |
| | | VYFCQQGNTLPYTFGQGTKL | |
| | | EIKTTTPAPRPPTPAPTIAS | |
| | | QPLSLRPEACRPAAGGAVHT | |
| | | RGLDFACDIYIWAPLAGTCG | |
| | | VLLLSLVITLYCKRGRKKLL | |
| | | YIFKQPFMRPVQTTQEEDGC | |
| | | SCRFPEEEEGGCELRVKFSR | |
| | | SADAPAYKQGQNQLYNELNL | |
| | | GRREEYDVLDKRRGRDPEMG | |
| | | GKPRRKNPQEGLYNELQKDK | |
| | | MAEAYSEIGMKGERRRGKGH | |
| | | DGLYQGLSTATKDTYDALHM | |
| | | QALPPR | |
| CD19 | CAR 4 | MALPVTALLLPLALLLHAAR | 241 |
| | | PQVQLQESGPGLVKPSETLS | |
| | | LTCTVSGVSLPDYGVSWIRQ | |
| | | PPGKGLEWIGVIWGSETTYY | |
| | | QSSLKSRVTISKDNSKNQVS | |
| | | LKLSSVTAADTAVYYCAKHY | |
| | | YYGGSYAMDYWGQGTLVTVS | |
| | | SGGGGSGGGGSGGGGSEIVM | |
| | | TQSPATLSLSPGERATLSCR | |
| | | ASQDISKYLNWYQQKPGQAP | |
| | | RLLIYHTSRLHSGIPARFSG | |
| | | SGSGTDYTLTISSLQPEDFA | |
| | | VYFCOOGNTLPYTFGQGTKL | |
| | | EIKTTTPAPRPPTPAPTIAS | |
| | | QPLSLRPEACRPAAGGAVHT | |
| | | RGLDFACDIYIWAPLAGTCG | |
| | | VLLLSLVITLYCKRGRKKLL | |
| | | YIFKQPFMRPVQTTQEEDGC | |
| | | SCRFPEEEEGGCELRVKFSR | |
| | | SADAPAYKQGQNQLYNELNL | |
| | | GRREEYDVLDKRRGRDPEMG | |
| | | GKPRRKNPQEGLYNELQKDK | |
| | | MAEAYSEIGMKGERRRGKGH | |
| | | DGLYOGLSTATKDTYDALHM | |
| | | QALPPR | |
| CD19 | CAR 5 | MALPVTALLLPLALLLHAAR | 242 |
| | | PEIVMTQSPATLSLSPGERA | |
| | | TLSCRASQDISKYLNWYQQK | |
| | | PGQAPRLLIYHTSRLHSGIP | |
| | | ARFSGSGSGTDYTLTISSLQ | |
| | | PEDFAVYFCQQGNTLPYTFG | |
| | | QGTKLEIKGGGGSGGGGSGG | |
| | | GGSGGGGSQVQLQESGPGLV | |
| | | KPSETLSLTCTVSGVSLPDY | |
| | | GVSWIRQPPGKGLEWIGVIW | |
| | | GSETTYYSSSLKSRVTISKD | |
| | | NSKNQVSLKLSSVTAADTAV | |
| | | YYCAKHYYYGGSYAMDYWGQ | |
| | | GTLVTVSSTTTPAPRPPTPA | |
| | | PTIASQPLSLRPEACRPAAG | |
| | | GAVHTRGLDFACDIYIWAPL | |
| | | AGTCGVLLLSLVITLYCKRG | |
| | | RKKLLYIFKQPFMRPVQTTQ | |
| | | EEDGCSCRFPEEEEGGCELR | |
| | | VKFSRSADAPAYKQGQNQLY | |
| | | NELNLGRREEYDVLDKRRGR | |
| | | DPEMGGKPRRKNPQEGLYNE | |
| | | LQKDKMAEAYSEIGMKGERR | |
| | | RGKGHDGLYQGLSTATKDTY | |
| | | DALHMQALPPR | |
| CD19 | CAR 6 | MALPVTALLLPLALLLHAAR | 243 |
| | | PEIVMTQSPATLSLSPGERA | |
| | | TLSCRASQDISKYLNWYQQK | |
| | | PGQAPRLLIYHTSRLHSGIP | |
| | | ARFSGSGSGTDYTLTISSLQ | |
| | | PEDFAVYFCQQGNTLPYTFG | |
| | | QGTKLEIKGGGGSGGGGSGG | |
| | | GGSGGGGSQVQLQESGPGLV | |
| | | KPSETLSLTCTVSGVSLPDY | |
| | | GVSWIRQPPGKGLEWIGVIW | |
| | | GSETTYYQSSLKSRVTISKD | |
| | | NSKNQVSLKLSSVTAADTAV | |
| | | YYCAKHYYYGGSYAMDYWGQ | |
| | | GTLVTVSSTTTPAPRPPTPA | |
| | | PTIASQPLSLRPEACRPAAG | |
| | | GAVHTRGLDFACDIYIWAPL | |
| | | AGTCGVLLLSLVITLYCKRG | |
| | | RKKLLYIFKQPFMRPVQTTQ | |
| | | EEDGCSCRFPEEEEGGCELR | |
| | | VKFSRSADAPAYKQGQNQLY | |
| | | NELNLGRREEYDVLDKRRGR | |
| | | DPEMGGKPRRKNPQEGLYNE | |
| | | LQKDKMAEAYSEIGMKGERR | |
| | | RGKGHDGLYQGLSTATKDTY | |
| | | DALHMQALPPR | |
| CD19 | CAR 7 | MALPVTALLLPLALLLHAAR | 244 |
| | | PQVQLQESGPGLVKPSETLS | |
| | | LTCTVSGVSLPDYGVSWIRQ | |
| | | PPGKGLEWTGVIWGSETTYY | |
| | | SSSLKSRVTISKDNSKNQVS | |
| | | LKLSSVTAADTAVYYCAKHY | |
| | | YYGGSYAMDYWGQGTLVTVS | |
| | | SGGGGSGGGGSGGGGSGGGG | |
| | | SEIVMTQSPATLSLSPGERA | |
| | | TLSCRASQDISKYLNWYQQK | |
| | | PGQAPRLLIYHTSRLHSGIP | |
| | | ARFSGSGSGTDYTLTISSLQ | |
| | | PEDFAVYFCQQGNTLPYTFG | |
| | | QGTKLEIKTTTPAPRPPTPA | |
| | | PTIASQPLSLRPEACRPAAG | |
| | | GAVHTRGLDFACDIYIWAPL | |
| | | AGTCGVLLLSLVITLYCKRG | |
| | | RKKLLYIFKQPFMRPVQTTQ | |
| | | EEDGCSCRFPEEEEGGCELR | |
| | | VKFSRSADAPAYKQGQNQLY | |
| | | NELNLGRREEYDVLDKRRGR | |
| | | DPEMGGKPRRKNPQEGLYNE | |
| | | LQKDKMAEAYSEIGMKGERR | |
| | | RGKGHDGLYQGLSTATKDTY | |
| | | DALHMQALPPR | |
| CD19 | CAR 8 | MALPVTALLLPLALLLHAAR | 245 |
| | | PQVQLQESGPGLVKPSETLS | |
| | | LTCTVSGVSLPDYGVSWIRQ | |
| | | PPGKGLEWIGVIWGSETTYY | |
| | | QSSLKSRVTISKDNSKNQVS | |
| | | LKLSSVTAADTAVYYCAKHY | |
| | | YYGGSYAMDYWGQGTLVTVS | |
| | | SGGGGSGGGGSGGGGSGGGG | |
| | | SEIVMTQSPATLSLSPGERA | |
| | | TLSCRASQDISKYLNWYQQK | |
| | | PGQAPRLLIYHTSRLHSGIP | |
| | | ARFSGSGSGTDYTLTISSLQ | |
| | | PEDFAVYFCQQGNTLPYTFG | |
| | | QGTKLEIKTTTPAPRPPTPA | |
| | | PTIASQPLSLRPEACRPAAG | |
| | | GAVHTRGLDFACDIYIWAPL | |
| | | AGTCGVLLLSLVITLYCKRG | |
| | | RKKLLYIFKQPFMRPVQTTQ | |
| | | EEDGCSCRFPEEEEGGCELR | |
| | | VKFSRSADAPAYKQGQNQLY | |
| | | NELNLGRREEYDVLDKRRGR | |
| | | DPEMGGKPRRKNPQEGLYNE | |
| | | LQKDKMAEAYSEIGMKGERR | |
| | | RGKGHDGLYQGLSTATKDTY | |
| | | DALHMQALPPR | |
| CD19 | CAR 9 | MALPVTALLLPLALLLHAAR | 246 |
| | | PEIVMTQSPATLSLSPGERA | |
| | | TLSCRASQDISKYLNWYQQK | |
| | | PGQAPRLLIYHTSRLHSGIP | |
| | | ARFSGSGSGTDYTLTISSLQ | |
| | | PEDFAVYFCQQGNTLPYTFG | |
| | | QGTKLEIKGGGGSGGGGSGG | |
| | | GGSGGGGSQVQLQESGPGLV | |
| | | KPSETLSLTCTVSGVSLPDY | |
| | | GVSWIRQPPGKGLEWIGVIW | |
| | | GSETTYYNSSLKSRVTISKD | |
| | | NSKNQVSLKLSSVTAADTAV | |
| | | YYCAKHYYYGGSYAMDYWGQ | |
| | | GTLVTVSSTTTPAPRPPTPA | |
| | | PTIASQPLSLRPEACRPAAG | |
| | | GAVHTRGLDFACDIYIWAPL | |
| | | AGTCGVLLLSLVITLYCKRG | |
| | | RKKLLYIFKQPFMRPVQTTQ | |
| | | EEDGCSCRFPEEEEGGCELR | |
| | | VKFSRSADAPAYKQGQNQLY | |
| | | NELNLGRREEYDVLDKRRGR | |
| | | DPEMGGKPRRKNPQEGLYNE | |
| | | LQKDKMAEAYSEIGMKGERR | |
| | | RGKGHDGLYQGLSTATKDTY | |
| | | DALHMQALPPR | |
| CD19 | CAR 10 | MALPVTALLLPLALLLHAAR | 247 |
| | | PEIVMTQSPATLSLSPGERA | |
| | | TLSCRASQDISKYLNWYQQK | |
| | | PGQAPRLLIYHTSRLHSGIP | |
| | | ARFSGSGSGTDYTLTISSLQ | |
| | | PEDFAVYFCQQGNTLPYTFG | |
| | | QGTKLEIKGGGGSGGGGSGG | |
| | | GGSGGGGSQVQLQESGPGLV | |
| | | KPSETLSLTCTVSGVSLPDY | |
| | | GVSWIRQPPGKGLEWIGVIW | |
| | | GSETTYYNSSLKSRVTISKD | |
| | | NSKNOVSLKLSSVTAADTAV | |
| | | YYCAKHYYYGGSYAMDYVVG | |
| | | QGTLVTVSSTTTPAPRPPTP | |
| | | APTIASQPLSLRPEACRPAA | |
| | | GGAVHTRGLDFACDIYIWAP | |
| | | LAGTCGVLLLSLVITLYCKR | |
| | | GRKKLLYIFKQPFMRPVQTT | |
| | | QEEDGCSCRFPEEEEGGCEL | |
| | | RVKFSRSADAPAYKQGQNQL | |
| | | YNELNLGRREEYDVLDKRRG | |
| | | RDPEMGGKPRRKNPQEGLYN | |
| | | ELQKDKMAEAYSEIGMKGER | |
| | | RRGKGHDGLYQGLSTATKDT | |
| | | YDALHMQALPPR | |
| CD19 | CAR 11 | MALPVTALLLPLALLLHAAR | 248 |
| | | PQVQLQESGPGLVKPSETLS | |
| | | LTCTVSGVSLPDYGVSWIRQ | |
| | | PPGKGLEWIGVIWGSETTYY | |
| | | NSSLKSRVTISKDNSKNQVS | |
| | | LKLSSVTAADTAVYYCAKHY | |
| | | YYGGSYAMDYWGQGTLVTVS | |
| | | SGGGGSGGGGSGGGGSGGGG | |
| | | SEIVMTQSPATLSLSPGERA | |
| | | TLSCRASQDISKYLNWYQQK | |
| | | PGQAPRLLIYHTSRLHSGIP | |
| | | ARFSGSGSGTDYTLTISSLQ | |
| | | PEDFAVYFCQQGNTLPYTFG | |
| | | QGTKLEIKTTTPAPRPPTPA | |
| | | PTIASQPLSLRPEACRPAAG | |
| | | GAVHTRGLDFACDIYIWAPL | |
| | | AGTCGVLLLSLVITLYCKRG | |
| | | RKKLLYIFKQPFMRPVQ | |
| | | TTQEEDGCSCRFPEEEEGGC | |
| | | ELRVKFSRSADAPAYKQGQN | |
| | | QLYNELNLGRREEYDVLDKR | |
| | | RGRDPEMGGKPRRKNPQEGL | |
| | | YNELQKDKMAEAYSEIGMKG | |
| | | ERRRGKGHDGLYQGLSTATK | |
| | | DTYDALHMQALPPR | |
| CD19 | CAR 12 | MALPVTALLLPLALLLHAAR | 249 |
| | | PEIVMTQSPATLSLSPGERA | |
| | | TLSCRASQDISKYLNWYQQK | |
| | | PGQAPRLLIYHTSRLHSGIP | |
| | | ARFSGSGSGTDYTLTISSLQ | |
| | | PEDFAVYFCQQGNTLPYTFG | |
| | | QGTKLEIKGGGGSGGGGSGG | |
| | | GGSQVQLQESGPGLVKPSET | |
| | | LSLTCTVSGVSLPDYGVSWI | |
| | | RQPPGKGLEWIGVIWGSETT | |
| | | YYNSSLKSRVTISKDNSKNQ | |
| | | VSLKLSSVTAADTAVYYCAK | |
| | | HYYYGGSYAMDYWGQGTLVT | |
| | | VSSTTTPAPRPPTPAPTIAS | |
| | | QPLSLRPEACRPAAGGAVHT | |
| | | RGLDFACDIYIWAPLAGTCG | |
| | | VLLLSLVITLYCKRGRKKLL | |
| | | YIFKQPFMRPVQTTQEEDGC | |
| | | SCRFPEEEEGGCELRVKFSR | |
| | | SADAPAYKQGQNQLYNELNL | |
| | | GRREEYDVLDKRRGRDPEMG | |
| | | GKPRRKNPQEGLYNELQKDK | |
| | | MAEAYSEIGMKGERRRGKGH | |
| | | DGLYQGLSTATKDTYDALHM | |
| | | QALPPR | |

In one aspect, a CAR, e.g., a CAR expressed by the cell of the disclosure, comprises a CAR molecule comprising an antigen binding domain that binds to BCMA, e.g., comprises a BCMA antigen binding domain (e.g., a murine, human or humanized antibody or antibody fragment that specifically binds to BCMA, e.g., human BCMA), a transmembrane domain, and an intracellular signaling domain (e.g., an intracellular signaling domain comprising a costimulatory domain and/or a primary signaling domain).

Exemplary CAR molecules of a CAR described herein are provided in Table 1 of WO2016/014565, which is incorporated by reference herein.

### Transmembrane Domains

With respect to the transmembrane domain, in various embodiments, a CAR can be designed to comprise a transmembrane domain that is attached to the extracellular domain of the CAR. A transmembrane domain can include one or more additional amino acids adjacent to the transmembrane region, e.g., one or more amino acid associated with the extracellular region of the protein from which the transmembrane was derived (e.g., 1, 2, 3, 4, 5, 6, 7, 8, 9, 10 up to 15 amino acids of the extracellular region) and/or one or more additional amino acids associated with the intracellular region of the protein from which the transmembrane protein is derived (e.g., 1, 2, 3, 4, 5, 6, 7, 8, 9, 10 up to 15 amino acids of the intracellular region). In one aspect, the transmembrane domain is one that is associated with one of the other domains of the CAR e.g., in one embodiment, the transmembrane domain may be from the same protein that the signaling domain, costimulatory domain or the hinge domain is derived from. In another aspect, the transmembrane domain is not derived from the same protein that any other domain of the CAR is derived from. In some instances, the transmembrane domain can be selected or modified by amino acid substitution to avoid binding of such domains to the transmembrane domains of the same or different surface membrane proteins, e.g., to minimize interactions with other members of the receptor complex. In one aspect, the transmembrane domain is capable of homodimerization with another CAR on the cell surface of a CAR-expressing cell. In a different aspect, the amino acid sequence of the transmembrane domain may be modified or substituted so as to minimize interactions with the binding domains of the native binding partner present in the same CAR-expressing cell.

The transmembrane domain may be derived either from a natural or from a recombinant source. Where the source is natural, the domain may be derived from any membrane-bound or transmembrane protein. In one aspect, the transmembrane domain is capable of signaling to the intracellular domain(s) whenever the CAR has bound to a target. A transmembrane domain of particular use in this disclosure may include at least the transmembrane region(s) of e.g., the alpha, beta or zeta chain of the T-cell receptor, CD28, CD27, CD3 epsilon, CD45, CD4, CD5, CD8, CD9, CD16, CD22, CD33, CD37, CD64, CD80, CD86, CD134, CD137, CD154. In some embodiments, a transmembrane domain may include at least the transmembrane region(s) of, e.g., KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2R beta, IL2R gamma, IL7R α, ITGAT, VLA1, CD49a, ITGA4, IA4, CD49D, ITGA6, VLA-6, CD49f, ITGAD, CD 11d, ITGAE, CD103, ITGAL, CD 11a, LFA-1, ITGAM, CD 11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKG2D, NKG2C.

In some instances, the transmembrane domain can be attached to the extracellular region of the CAR, e.g., the antigen binding domain of the CAR, via a hinge, e.g., a hinge from a human protein. For example, in one embodiment, the hinge can be a human Ig (immunoglobulin) hinge (e.g., an IgG4 hinge, an IgD hinge), a GS linker (e.g., a GS linker described herein), a KIR2DS2 hinge or a CD8a hinge. In one embodiment, the hinge or spacer comprises (e.g., consists of) the amino acid sequence of SEQ ID NO: 250. In one aspect, the transmembrane domain comprises (e.g., consists of) a transmembrane domain of SEQ ID NO: 251.

In certain embodiments, the encoded transmembrane domain comprises an amino acid sequence of a CD8 transmembrane domain having at least one, two or three modifications but not more than 20, 10 or 5 modifications of the amino acid sequence of SEQ ID NO: 251, or a sequence with at least 95% identity to the amino acid sequence of SEQ ID NO: 251. In one embodiment, the encoded transmembrane domain comprises the sequence of SEQ ID NO: 251.

In other embodiments, the nucleic acid molecule encoding the CAR comprises a nucleotide sequence of a CD8 transmembrane domain, e.g., comprising the sequence of SEQ ID NO: 252 or SEQ ID NO: 289, or a sequence with at least 95% identity thereof.

In certain embodiments, the encoded antigen binding domain is connected to the transmembrane domain by a hinge region. In one embodiment, the encoded hinge region comprises the amino acid sequence of a CD8 hinge, e.g., SEQ ID NO: 250; or the amino acid sequence of an IgG4 hinge, e.g., SEQ ID NO: 253 or a sequence with at least 95% identity to SEQ ID NO: 250 or SEQ ID NO: 253. In other embodiments, the nucleic acid sequence encoding the hinge region comprises the sequence of SEQ ID NO: 254 or SEQ ID NO: 255, corresponding to a CD8 hinge or an IgG4 hinge, respectively, or a sequence with at least 95% identity to SEQ ID NO: 254 or 255.

In one aspect, the hinge or spacer comprises an IgG4 hinge. For example, in one embodiment, the hinge or spacer comprises a hinge of the amino acid sequence ESKYGPPCPPCPAPEFLGGPSVFLFPPKPKDTLMISRTPEVTCVVVDVSQEDPEVQFNWY VDGVEVHNAKTKPREEQFNSTYRVVSVLTVLHQDWLNGKEYKCKVSNKGLPSSIEKTIS KAKGQPREPQVYTLPPSQEEMTKNQVSLTCLVKGFYPSDIAVEWESNGQPENNYKTTPP VLDSDGSFFLYSRLTVDKSRWQEGNVFSCSVMHEALHNHYTQKSLSLSLGKM (SEQ ID NO: 253). In some embodiments, the hinge or spacer comprises a hinge encoded by the nucleotide sequence of

(SEQ ID NO: 255)
GAGAGCAAGTACGGCCCTCCCTGCCCCCCTTGCCC
TGCCCCCGAGTTCCTGGGCGGACCCAGCGTGTTCC
TGTTCCCCCCCAAGCCCAAGGACACCCTGATGATC
AGCCGGACCCCCGAGGTGACCTGTGTGGTGGTGGA
CGTGTCCCAGGAGGACCCCGAGGTCCAGTTCAACT
GGTACGTGGACGGCGTGGAGGTGCACAACGCCAAG
ACCAAGCCCCGGGAGGAGCAGTTCAATAGCACCTA
CCGGGTGGTGTCCGTGCTGACCGTGCTGCACCAGG
ACTGGCTGAACGGCAAGGAATACAAGTGTAAGGTG
TCCAACAAGGGCCTGCCCAGCAGCATCGAGAAAAC
CATCAGCAAGGCCAAGGGCCAGCCTCGGGAGCCCC
AGGTGTACACCCTGCCCCCTAGCCAAGAGGAGATG
ACCAAGAACCAGGTGTCCCTGACCTGCCTGGTGAA
GGGCTTCTACCCCAGCGACATCGCCGTGGAGTGGG
AGAGCAACGGCCAGCCCGAGAACAACTACAAGACC
ACCCCCCCTGTGCTGGACAGCGACGGCAGCTTCTT
CCTGTACAGCCGGCTGACCGTGGACAAGAGCCGGT
GGCAGGAGGGCAACGTCTTTAGCTGCTCCGTGATG
CACGAGGCCCTGCACAACCACTACACCCAGAAGAG
CCTGAGCCTGTCCCTGGGCAAGATG.

In one aspect, the hinge or spacer comprises an IgD hinge. For example, in one embodiment, the hinge or spacer comprises a hinge of the amino acid sequence of RWPESPKAQASSVPTAQPQAEGSLAKATTAPATTRNTGRGGEEKKKEKEKEEQEERET KTPECPSHTQPLGVYLLTPAVQDLWLRDKATFTCFVVGSDLKDAHLTWEVAGKVPTGG VEEGLLERHSNGSQSQHSRLTLPRSLWNAGTSVTCTLNHPSLPPQRLMALREPAAQAPV KLSLNLLASSDPPEAASWLLCEVSGFSPPNILLMWLEDQREVNTSGFAPARPPPQPGSTT FWAWSVLRVPAPPSPQPATYTCVVSHEDSRTLLNASRSLEVSYVTDH (SEQ ID NO: 256). In some embodiments, the hinge or spacer comprises a hinge encoded by the nucleotide sequence of

(SEQ ID NO: 257)
AGGTGGCCCGAAAGTCCCAAGGCCCAGGCATCTAG
TGTTCCTACTGCACAGCCCCAGGCAGAAGGCAGCC
TAGCCAAAGCTACTACTGCACCTGCCACTACGCGC
AATACTGGCCGTGGCGGGGAGGAGAAGAAAAAGGA
GAAAGAGAAAGAAGAACAGGAAGAGAGGGAGACCA
AGACCCCTGAATGTCCATCCCATACCCAGCCGCTG
GGCGTCTATCTCTTGACTCCCGCAGTACAGGACTT
GTGGCTTAGAGATAAGGCCACCTTTACATGTTTCG
TCGTGGGCTCTGACCTGAAGGATGCCCATTTGACT
TGGGAGGTTGCCGGAAAGGTACCCACAGGGGGGGT
TGAGGAAGGGTTGCTGGAGCGCCATTCCAATGGCT
CTCAGAGCCAGCACTCAAGACTCACCCTTCCGAGA
TCCCTGTGGAACGCCGGGACCTCTGTCACATGTAC
TCTAAATCATCCTAGCCTGCCCCCACAGCGTCTGA
TGGCCCTTAGAGAGCCAGCCGCCCAGGCACCAGTT
AAGCTTAGCCTGAATCTGCTCGCCAGTAGTGATCC
CCCAGAGGCCGCCAGCTGGCTCTTATGCGAAGTGT
CCGGCTTTAGCCCGCCCAACATCTTGCTCATGTGG
CTGGAGGACCAGCGAGAAGTGAACACCAGCGGCTT
CGCTCCAGCCCGGCCCCCACCCCAGCCGGGTTCTA
CCACATTCTGGGCCTGGAGTGTCTTAAGGGTCCCA
GCACCACCTAGCCCCCAGCCAGCCACATACACCTG
TGTTGTGTCCCATGAAGATAGCAGGACCCTGCTAA
ATGCTTCTAGGAGTCTGGAGGTTTCCTACGTGACT
GACCATT.

In one aspect, the transmembrane domain may be recombinant, in which case it will comprise predominantly hydrophobic residues such as leucine and valine. In one aspect a triplet of phenylalanine, tryptophan and valine can be found at each end of a recombinant transmembrane domain.

Optionally, a short oligo- or polypeptide linker, between 2 and 10 amino acids in length may form the linkage between the transmembrane domain and the cytoplasmic region of the CAR. A glycine-serine doublet provides a particularly suitable linker. For example, in one aspect, the linker comprises the amino acid sequence of GGGGSGGGGS (SEQ ID NO: 258). In some embodiments, the linker is encoded by the nucleotide sequence of

(SEQ ID NO: 259)
GGTGGCGGAGGTTCTGGAGGTGGAGGTTCC.

In one aspect, the hinge or spacer comprises a KIR2DS2 hinge.

### Signaling Domains

In embodiments of the disclosure having an intracellular signaling domain, such a domain can contain, e.g., one or more of a primary signaling domain and/or a costimulatory signaling domain. In some embodiments, the intracellular signaling domain comprises a sequence encoding a primary signaling domain. In some embodiments, the intracellular signaling domain comprises a costimulatory signaling domain. In some embodiments, the intracellular signaling domain comprises a primary signaling domain and a costimulatory signaling domain.

The intracellular signaling sequences within the cytoplasmic portion of the CAR of the disclosure may be linked to each other in a random or specified order. Optionally, a short oligo- or polypeptide linker, for example, between 2 and 10 amino acids (e.g., 2, 3, 4, 5, 6, 7, 8, 9, or 10 amino acids) in length may form the linkage between intracellular signaling sequences. In one embodiment, a glycine-serine doublet can be used as a suitable linker. In one embodiment, a single amino acid, e.g., an alanine, a glycine, can be used as a suitable linker.

In one aspect, the intracellular signaling domain is designed to comprise two or more, e.g., 2, 3, 4, 5, or more, costimulatory signaling domains. In an embodiment, the two or more, e.g., 2, 3, 4, 5, or more, costimulatory signaling domains, are separated by a linker molecule, e.g., a linker molecule described herein. In one embodiment, the intracellular signaling domain comprises two costimulatory signaling domains. In some embodiments, the linker molecule is a glycine residue. In some embodiments, the linker is an alanine residue.

### Primary Signaling Domains

A primary signaling domain regulates primary activation of the TCR complex either in a stimulatory way, or in an inhibitory way. Primary intracellular signaling domains that act in a stimulatory manner may contain signaling motifs, which are known as immunoreceptor tyrosine-based activation motifs or ITAMs.

Examples of ITAM containing primary intracellular signaling domains that are of particular use in the disclosure include those of CD3 zeta, common FcR gamma (FCER1G), Fc gamma RIIa, FcR beta (Fc Epsilon R1b), CD3 gamma, CD3 delta, CD3 epsilon, CD79a, CD79b, DAP10, and DAP12. In one embodiment, a CAR of the disclosure comprises an intracellular signaling domain, e.g., a primary signaling domain of CD3-zeta.

In one embodiment, the encoded primary signaling domain comprises a functional signaling domain of CD3 zeta. The encoded CD3 zeta primary signaling domain can comprise an amino acid sequence having at least one, two or three modifications but not more than 20, 10 or 5 modifications of the amino acid sequence of SEQ ID NO: 260 or SEQ ID NO: 261, or a sequence with at least 95% identity to the amino acid sequence of SEQ ID NO: 260 or SEQ ID NO: 261. In some embodiments, the encoded primary signaling domain comprises the sequence of SEQ ID NO: 260 or SEQ ID NO: 261. In other embodiments, the nucleic acid sequence encoding the primary signaling domain comprises the sequence of SEQ ID NO: 262, SEQ ID NO: 291, or SEQ ID NO: 263, or a sequence with at least 95% identity thereof.

### Costimulatory Signaling Domains

In some embodiments, the encoded intracellular signaling domain comprises a costimulatory signaling domain. For example, the intracellular signaling domain can comprise a primary signaling domain and a costimulatory signaling domain. In some embodiments, the encoded costimulatory signaling domain comprises a functional signaling domain of a protein chosen from one or more of CD27, CD28, 4-1BB (CD137), OX40, CD30, CD40, PD-1, ICOS, lymphocyte function-associated antigen-I (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD1Id, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, or NKG2D.

In certain embodiments, the encoded costimulatory signaling domain comprises an amino acid sequence having at least one, two or three modifications but not more than 20, 10 or 5 modifications of the amino acid sequence of SEQ ID NO: 264 or SEQ ID NO: 265, or a sequence with at least 95% identity to the amino acid sequence of SEQ ID NO: 264 or SEQ ID NO: 265. In one embodiment, the encoded costimulatory signaling domain comprises the sequence of SEQ ID NO: 264 or SEQ ID NO: 265. In other embodiments, the nucleic acid sequence encoding the costimulatory signaling domain comprises the sequence of SEQ ID NO: 266, SEQ ID NO: 290, or SEQ ID NO: 267, or a sequence with at least 95% identity thereof.

In other embodiments, the encoded intracellular domain comprises the sequence of SEQ ID NO: 264 or SEQ ID NO: 265 and the sequence of SEQ ID NO: 260 or SEQ ID NO: 261, wherein the sequences comprising the intracellular signaling domain are expressed in the same frame and as a single polypeptide chain.

In certain embodiments, the nucleic acid sequence encoding the intracellular signaling domain comprises the sequence of SEQ ID NO: 266, SEQ ID NO: 290, or SEQ ID NO: 267, or a sequence with at least 95% identity thereof, and the sequence of SEQ ID NO: 262, SEQ ID NO: 291, or SEQ ID NO: 263, or a sequence with at least 95% identity thereof.

In some embodiments, the nucleic acid molecule further encodes a leader sequence. In one embodiment, the leader sequence comprises the sequence of SEQ ID NO: 268.

In one aspect, the intracellular signaling domain is designed to comprise the signaling domain of CD3-zeta and the signaling domain of CD28. In one aspect, the intracellular signaling domain is designed to comprise the signaling domain of CD3-zeta and the signaling domain of 4-1BB. In one aspect, the signaling domain of 4-1BB is a signaling domain of SEQ ID NO: 264. In one aspect, the signaling domain of CD3-zeta is a signaling domain of SEQ ID NO: 260.

In one aspect, the intracellular signalling domain is designed to comprise the signalling domain of CD3-zeta and the signalling domain of CD27. In one aspect, the signalling domain of CD27 comprises the amino acid sequence of

(SEQ ID NO: 265)
QRRKYRSNKGESPVEPAEPCRYSCPREEEGSTIPIQEDYRKPEPACSP.

In one aspect, the signalling domain of CD27 is encoded by the nucleic acid sequence of

(SEQ ID NO: 267)
Caacgaaggaaatatagatcaaacaaaggagaaag
tcctgtggagcctgcagagccttgtcgttacagct
gccccagggaggaggagggcagcaccatccccatc
caggaggattaccgaaaaccggagcctgcctgctc
cccc.

### Vectors

In another aspect, the disclosure pertains to a vector comprising a nucleic acid sequence encoding a CAR described herein. In one embodiment, the vector is chosen from a DNA vector, an RNA vector, a plasmid, a lentivirus vector, adenoviral vector, or a retrovirus vector. In one embodiment, the vector is a lentivirus vector. These vectors or portions thereof may, among other things, be used to create template nucleic acids, as described herein for use with the CRISPR systems as described herein. Alternatively, the vectors may be used to deliver nucleic acid directly to the cell, e.g., the immune effector cell, e.g., the T cell, e.g., the allogeneic T cell, independent of the CRISPR system.

The present disclosure also provides vectors in which a DNA of the present disclosure is inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. A retroviral vector may also be, e.g., a gammaretroviral vector. A gammaretroviral vector may include, e.g., a promoter, a packaging signal (W), a primer binding site (PBS), one or more (e.g., two) long terminal repeats (LTR), and a transgene of interest, e.g., a gene encoding a CAR. A gammaretroviral vector may lack viral structural gens such as gag, pol, and env. Exemplary gammaretroviral vectors include Murine Leukemia Virus (MLV), Spleen-Focus Forming Virus (SFFV), and Myeloproliferative Sarcoma Virus (MPSV), and vectors derived therefrom. Other gammaretroviral vectors are described, e.g., in Tobias Maetzig et al., “Gammaretroviral Vectors: Biology, Technology and Application” Viruses. 2011 June; 3(6): 677-713.

In another embodiment, the vector comprising the nucleic acid encoding the desired CAR of the disclosure is an adenoviral vector (A5/35). In another embodiment, the expression of nucleic acids encoding CARs can be accomplished using of transposons such as sleeping beauty, crisper, CAS9, and zinc finger nucleases. See below June et al. 2009 Nature Reviews Immunology 9.10: 704-716, is incorporated herein by reference.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Disclosed herein are methods for producing an in vitro transcribed RNA CAR. The present disclosure also includes a CAR encoding RNA construct that can be directly transfected into a cell. A method for generating mRNA for use in transfection can involve in vitro transcription (IVT) of a template with specially designed primers, followed by polyA addition, to produce a construct containing 3′ and 5′ untranslated sequence (“UTR”), a 5′ cap and/or Internal Ribosome Entry Site (IRES), the nucleic acid to be expressed, and a polyA tail, typically 50-2000 bases in length (SEQ ID NO: 269). RNA so produced can efficiently transfect different kinds of cells. In one aspect, the template includes sequences for the CAR.

### Non-Viral Delivery Methods

In some aspects, non-viral methods can be used to deliver a nucleic acid encoding a CAR described herein into a cell or tissue or a subject.

In some embodiments, the non-viral method includes the use of a transposon (also called a transposable element). In some embodiments, a transposon is a piece of DNA that can insert itself at a location in a genome, for example, a piece of DNA that is capable of self-replicating and inserting its copy into a genome, or a piece of DNA that can be spliced out of a longer nucleic acid and inserted into another place in a genome. For example, a transposon comprises a DNA sequence made up of inverted repeats flanking genes for transposition.

In some embodiments, cells, e.g., T or NK cells, are generated that express a CAR described herein by using a combination of gene insertion using the SBTS and genetic editing using a nuclease (e.g., Zinc finger nucleases (ZFNs), Transcription Activator-Like Effector Nucleases (TALENs), the CRISPR/Cas system, or engineered meganuclease re-engineered homing endonucleases).

In some embodiments, cells of the disclosure, e.g., T or NK cells, e.g., allogeneic T cells, e.g., described herein, (e.g., that express a CAR described herein) are generated by contacting the cells with (a) a composition comprising one or more gRNA molecules, e.g., as described herein, and one or more Cas molecules, e.g., a Cas9 molecule, e.g., as described herein, and (b) nucleic acid comprising sequence encoding a CAR, e.g., described herein (such as a template nucleic acid molecule as described herein). Without being bound by theory, said composition of (a), above, will induce a break at or near the genomic DNA targeted by the targeting domain of the gRNA molecule(s), and the nucleic acid of (b) will incorporate, e.g., partially or wholly, into the genome at or near said break, such that upon integration, the encoded CAR molecule is expressed. In embodiments, expression of the CAR will be controlled by promoters or other regulatory elements endogenous to the genome (e.g., the promoter controlling expression from the gene in which the nucleic acid of (b) was inserted). In other embodiments, the nucleic acid of (b) further comprises a promoter and/or other regulatory elements, e.g., as described herein, e.g., an EF1-alpha promoter, operably linked to the sequence encoding the CAR, such that upon integration, expression of the CAR is controlled by that promoter and/or other regulatory elements. Additional features of the disclosure relating to use of CRISPR/Cas9 systems, e.g., as described herein, to direct incorporation of nucleic acid sequence encoding a CAR, e.g., as described herein, are described elsewhere in this application, e.g., in the section relating to gene insertion and homologous recombination. In embodiments, the composition of a) above is a composition comprising RNPs comprising the one or more gRNA molecules. In embodiments, RNPs comprising gRNAs targeting unique target sequences are introduced into the cell simultaneously, e.g., as a mixture of RNPs comprising the one or more gRNAs. In embodiments, RNPs comprising gRNAs targeting unique target sequences are introduced into the cell sequentially.

In some embodiments, use of a non-viral method of delivery permits reprogramming of cells, e.g., T or NK cells, and direct infusion of the cells into a subject. Advantages of non-viral vectors include but are not limited to the ease and relatively low cost of producing sufficient amounts required to meet a patient population, stability during storage, and lack of immunogenicity.

### Promoters

In one embodiment, the vector further comprises a promoter. In some embodiments, the promoter is chosen from an EF-1 promoter, a CMV IE gene promoter, an EF-1α promoter, an ubiquitin C promoter, or a phosphoglycerate kinase (PGK) promoter. In one embodiment, the promoter is an EF-1 promoter. In one embodiment, the EF-1 promoter comprises the sequence of SEQ ID NO: 270.

### Host Cells for CAR Expression

As noted above, in some aspects the disclosure pertains to a cell, e.g., an immune effector cell, (e.g., a population of cells, e.g., a population of immune effector cells) comprising a nucleic acid molecule, a CAR polypeptide molecule, or a vector as described herein.

In certain aspects of the present disclosure, immune effector cells, e.g., T cells, can be obtained from a unit of blood collected from a subject using any number of techniques known to the skilled artisan, such as Ficoll™ separation. In one preferred aspect, cells from the circulating blood of an individual are obtained by apheresis. The apheresis product typically contains lymphocytes, including T cells, monocytes, granulocytes, B cells, other nucleated white blood cells, red blood cells, and platelets. In one aspect, the cells collected by apheresis may be washed to remove the plasma fraction and, optionally, to place the cells in an appropriate buffer or media for subsequent processing steps. In one embodiment, the cells are washed with phosphate buffered saline (PBS). In an alternative embodiment, the wash solution lacks calcium and may lack magnesium or may lack many if not all divalent cations.

Initial activation steps in the absence of calcium can lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca-free, Mg-free PBS, PlasmaLyte A, or other saline solution with or without buffer. Alternatively, the undesirable components of the apheresis sample may be removed and the cells directly resuspended in culture media.

It is recognized that the methods of the application can utilize culture media conditions comprising 5% or less, for example 2%, human AB serum, and employ known culture media conditions and compositions, for example those described in Smith et al., “Ex vivo expansion of human T cells for adoptive immunotherapy using the novel Xeno-free CTS Immune Cell Serum Replacement” Clinical & Translational Immunology (2015) 4, e31; doi:10.1038/cti.2014.31.

In one aspect, T cells are isolated from peripheral blood lymphocytes by lysing the red blood cells and depleting the monocytes, for example, by centrifugation through a PERCOLL™ gradient or by counterflow centrifugal elutriation.

The methods described herein can include, e.g., selection of a specific subpopulation of immune effector cells, e.g., T cells, that are a T regulatory cell-depleted population, CD25+ depleted cells, using, e.g., a negative selection technique, e.g., described herein. Preferably, the population of T regulatory depleted cells contains less than 30%, 25%, 20%, 15%, 10%, 5%, 4%, 3%, 2%, 1% of CD25+ cells.

In one embodiment, T regulatory cells, e.g., CD25+ T cells, are removed from the population using an anti-CD25 antibody, or fragment thereof, or a CD25-binding ligand, IL-2. In one embodiment, the anti-CD25 antibody, or fragment thereof, or CD25-binding ligand is conjugated to a substrate, e.g., a bead, or is otherwise coated on a substrate, e.g., a bead. In one embodiment, the anti-CD25 antibody, or fragment thereof, is conjugated to a substrate as described herein.

In one embodiment, the T regulatory cells, e.g., CD25+ T cells, are removed from the population using CD25 depletion reagent from Miltenyi™. In one embodiment, the ratio of cells to CD25 depletion reagent is 1e7 cells to 20 uL, or 1e7 cells to 15 uL, or 1e7 cells to 10 uL, or 1e7 cells to 5 uL, or 1e7 cells to 2.5 uL, or 1e7 cells to 1.25 uL. In one embodiment, e.g., for T regulatory cells, e.g., CD25+ depletion, greater than 500 million cells/ml is used. In a further aspect, a concentration of cells of 600, 700, 800, or 900 million cells/ml is used.

In one embodiment, the population of immune effector cells to be depleted includes about 6×10⁹ CD25+ T cells. In other aspects, the population of immune effector cells to be depleted include about 1×10⁹ to 1×10¹⁰ CD25+ T cell, and any integer value in between. In one embodiment, the resulting population T regulatory depleted cells has 2×10⁹ T regulatory cells, e.g., CD25+ cells, or less (e.g., 1×10⁹, 5×10⁸, 1×10⁸, 5×10⁷, 1×10⁷, or less CD25+ cells).

In one embodiment, the T regulatory cells, e.g., CD25+ cells, are removed from the population using the CliniMAC system with a depletion tubing set, such as, e.g., tubing 162-01. In one embodiment, the CliniMAC system is run on a depletion setting such as, e.g., DEPLETION2.1.

Without wishing to be bound by a particular theory, decreasing the level of negative regulators of immune cells (e.g., decreasing the number of unwanted immune cells, e.g., T_{REG} cells), in a subject prior to apheresis or during manufacturing of a CAR-expressing cell product can reduce the risk of subject relapse. For example, methods of depleting T_{REG} cells are known in the art. Methods of decreasing T_{REG} cells include, but are not limited to, cyclophosphamide, anti-GITR antibody (an anti-GITR antibody described herein), CD25-depletion, and combinations thereof.

In some embodiments, the manufacturing methods comprise reducing the number of (e.g., depleting) T_{REG} cells prior to manufacturing of the CAR-expressing cell. For example, manufacturing methods comprise contacting the sample, e.g., the apheresis sample, with an anti-GITR antibody and/or an anti-CD25 antibody (or fragment thereof, or a CD25-binding ligand), e.g., to deplete T_{REG} cells prior to manufacturing of the CAR-expressing cell (e.g., T cell, NK cell) product.

In an embodiment, a subject is pre-treated with one or more therapies that reduce T_{REG} cells prior to collection of cells for CAR-expressing cell product manufacturing, thereby reducing the risk of subject relapse to CAR-expressing cell treatment. In an embodiment, methods of decreasing T_{REG} cells include, but are not limited to, administration to the subject of one or more of cyclophosphamide, anti-GITR antibody, CD25-depletion, or a combination thereof. Administration of one or more of cyclophosphamide, anti-GITR antibody, CD25-depletion, or a combination thereof, can occur before, during or after an infusion of the CAR-expressing cell product.

In an embodiment, a subject is pre-treated with cyclophosphamide prior to collection of cells for CAR-expressing cell product manufacturing, thereby reducing the risk of subject relapse to CAR-expressing cell treatment. In an embodiment, a subject is pre-treated with an anti-GITR antibody prior to collection of cells for CAR-expressing cell product manufacturing, thereby reducing the risk of subject relapse to CAR-expressing cell treatment.

In one embodiment, the population of cells to be removed are neither the regulatory T cells or tumor cells, but cells that otherwise negatively affect the expansion and/or function of CART cells, e.g. cells expressing CD14, CD11b, CD33, CD15, or other markers expressed by potentially immune suppressive cells. In one embodiment, such cells are envisioned to be removed concurrently with regulatory T cells and/or tumor cells, or following said depletion, or in another order.

The methods described herein can include more than one selection step, e.g., more than one depletion step. Enrichment of a T cell population by negative selection can be accomplished, e.g., with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail can include antibodies to CD14, CD20, CD11b, CD16, HLA-DR, and CD8.

The methods described herein can further include removing cells from the population which express a tumor antigen, e.g., a tumor antigen that does not comprise CD25, e.g., CD19, CD30, CD38, CD123, CD20, CD14 or CD11b, to thereby provide a population of T regulatory depleted, e.g., CD25+ depleted, and tumor antigen depleted cells that are suitable for expression of a CAR, e.g., a CAR described herein. In one embodiment, tumor antigen expressing cells are removed simultaneously with the T regulatory, e.g., CD25+ cells. For example, an anti-CD25 antibody, or fragment thereof, and an anti-tumor antigen antibody, or fragment thereof, can be attached to the same substrate, e.g., bead, which can be used to remove the cells or an anti-CD25 antibody, or fragment thereof, or the anti-tumor antigen antibody, or fragment thereof, can be attached to separate beads, a mixture of which can be used to remove the cells. In other embodiments, the removal of T regulatory cells, e.g., CD25+ cells, and the removal of the tumor antigen expressing cells is sequential, and can occur, e.g., in either order.

Also provided are methods that include removing cells from the population which express a check point inhibitor, e.g., a check point inhibitor described herein, e.g., one or more of PD1+ cells, LAG3+ cells, and TIM3+ cells, to thereby provide a population of T regulatory depleted, e.g., CD25+ depleted cells, and check point inhibitor depleted cells, e.g., PD1+, LAG3+ and/or TIM3+ depleted cells. Exemplary check point inhibitors include B7-H1, B7-1, CD160, PTH, 2B4, PD1, TIM3, CEACAM (e.g., CEACAM-1, CEACAM-3 and/or CEACAM-5), LAG3, TIGIT, CTLA-4, BTLA and LAIR1. In one embodiment, check point inhibitor expressing cells are removed simultaneously with the T regulatory, e.g., CD25+ cells. For example, an anti-CD25 antibody, or fragment thereof, and an anti-check point inhibitor antibody, or fragment thereof, can be attached to the same bead which can be used to remove the cells, or an anti-CD25 antibody, or fragment thereof, and the anti-check point inhibitor antibody, or fragment there, can be attached to separate beads, a mixture of which can be used to remove the cells. In other embodiments, the removal of T regulatory cells, e.g., CD25+ cells, and the removal of the check point inhibitor expressing cells is sequential, and can occur, e.g., in either order.

Methods described herein can include a positive selection step. For example, T cells can isolated by incubation with anti-CD3/anti-CD28 (e.g., 3×28)-conjugated beads, such as DYNABEADS® M-450 CD3/CD28 T, for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another embodiment, the time period is 10 to 24 hours, e.g., 24 hours. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points.

In one embodiment, a T cell population can be selected that expresses one or more of IFN-γ, TNFα, IL-17A, IL-2, IL-3, IL-4, GM-CSF, IL-10, IL-13, granzyme B, and perforin, or other appropriate molecules, e.g., other cytokines. Methods for screening for cell expression can be determined, e.g., by the methods described in PCT Publication No.: WO 2013/126712.

For isolation of a desired population of cells by positive or negative selection, the concentration of cells and surface (e.g., particles such as beads) can be varied. In certain aspects, it may be desirable to significantly decrease the volume in which beads and cells are mixed together (e.g., increase the concentration of cells), to ensure maximum contact of cells and beads. For example, in one aspect, a concentration of 10 billion cells/ml, 9 billion/ml, 8 billion/ml, 7 billion/ml, 6 billion/ml, or 5 billion/ml is used. In one aspect, a concentration of 1 billion cells/ml is used. In yet one aspect, a concentration of cells from 75, 80, 85, 90, 95, or 100 million cells/ml is used. In further aspects, concentrations of 125 or 150 million cells/ml can be used.

Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (e.g., leukemic blood, tumor tissue, etc.). Such populations of cells may have therapeutic value and would be desirable to obtain. For example, using high concentration of cells allows more efficient selection of CD8+ T cells that normally have weaker CD28 expression.

In a related aspect, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In one aspect, the concentration of cells used is 5×10⁶/ml. In other aspects, the concentration used can be from about 1×10⁵/ml to 1×10⁶/ml, and any integer value in between.

In other aspects, the cells may be incubated on a rotator for varying lengths of time at varying speeds at either 2-10° C. or at room temperature.

T cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In certain aspects, cryopreserved cells are thawed and washed as described herein and allowed to rest for one hour at room temperature prior to activation using the methods of the present disclosure.

Also contemplated in the context of the disclosure is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in immune effector cell therapy for any number of diseases or conditions that would benefit from immune effector cell therapy, such as those described herein. In one aspect, a blood sample or an apheresis is taken from a generally healthy subject. In certain aspects, a blood sample or an apheresis is taken from a generally healthy subject who is at risk of developing a disease, but who has not yet developed a disease, and the cells of interest are isolated and frozen for later use. In certain aspects, the T cells may be expanded, frozen, and used at a later time. In certain aspects, samples are collected from a patient shortly after diagnosis of a particular disease as described herein but prior to any treatments. In a further aspect, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation.

In a further aspect of the present disclosure, T cells are obtained from a patient directly following treatment that leaves the subject with functional T cells. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present disclosure to collect blood cells, including T cells, dendritic cells, or other cells of the hematopoietic lineage, during this recovery phase. Further, in certain aspects, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

In one embodiment, the immune effector cells expressing a CAR molecule, e.g., a CAR molecule described herein, are obtained from a subject that has received a low, immune enhancing dose of an mTOR inhibitor. In an embodiment, the population of immune effector cells, e.g., T cells, to be engineered to express a CAR, are harvested after a sufficient time, or after sufficient dosing of the low, immune enhancing, dose of an mTOR inhibitor, such that the level of PD1 negative immune effector cells, e.g., T cells, or the ratio of PD1 negative immune effector cells, e.g., T cells/PD1 positive immune effector cells, e.g., T cells, in the subject or harvested from the subject has been, at least transiently, increased.

In other embodiments, population of immune effector cells, e.g., T cells, which have, or will be engineered to express a CAR, can be treated ex vivo by contact with an amount of an mTOR inhibitor that increases the number of PD1 negative immune effector cells, e.g., T cells or increases the ratio of PD1 negative immune effector cells, e.g., T cells/PD1 positive immune effector cells, e.g., T cells.

In one embodiment, a T cell population is diaglycerol kinase (DGK)-deficient. DGK-deficient cells include cells that do not express DGK RNA or protein, or have reduced or inhibited DGK activity. DGK-deficient cells can be generated by genetic approaches, e.g., administering RNA-interfering agents, e.g., siRNA, shRNA, miRNA, to reduce or prevent DGK expression. Alternatively, DGK-deficient cells can be generated by treatment with DGK inhibitors described herein.

In one embodiment, a T cell population is Ikaros-deficient. Ikaros-deficient cells include cells that do not express Ikaros RNA or protein, or have reduced or inhibited Ikaros activity, Ikaros-deficient cells can be generated by genetic approaches, e.g., administering RNA-interfering agents, e.g., siRNA, shRNA, miRNA, to reduce or prevent Ikaros expression. Alternatively, Ikaros-deficient cells can be generated by treatment with Ikaros inhibitors, e.g., lenalidomide.

In embodiments, a T cell population is DGK-deficient and Ikaros-deficient, e.g., does not express DGK and Ikaros, or has reduced or inhibited DGK and Ikaros activity. Such DGK and Ikaros-deficient cells can be generated by any of the methods described herein.

In an embodiment, the NK cells are obtained from the subject. In another embodiment, the NK cells are an NK cell line, e.g., NK-92 cell line (Conkwest).

In some aspects, the cells of the disclosure (e.g., the immune effector cells of the disclosure, e.g., the CAR-expressing cells of the disclosure) are induced pluripotent stem cells (“iPSCs”) or embryonic stem cells (ESCs), or are T cells generated from (e.g., differentiated from) said iPSC and/or ESC. iPSCs can be generated, for example, by methods known in the art, from peripheral blood T lymphocytes, e.g., peripheral blood T lymphocytes isolated from a healthy volunteer. As well, such cells may be differentiated into T cells by methods known in the art. See e.g., Themeli M. et al., Nat. Biotechnol., 31, pp. 928-933 (2013); doi:10.1038/nbt.2678; WO2014/165707, the contents of each of which are incorporated herein by reference in their entirety.

In another embodiment, the compounds of Formula (I) or compounds of Embodiment 16, 17, or 35, or a pharmaceutically acceptable salt, hydrate, solvate, prodrug, stereoisomer, or tautomer thereof, of the present disclosure are used in combination with one or more of the therapeutic agents listed in Table 13 or listed in the patent and patent applications cited in Table 13, to treat cancer. Each publication listed in Table 13 is herein incorporated by reference in its entirety, including all structural formulae therein.

TABLE 13

| Second | Genetic | | Patents/Patent |
|---|---|---|---|
| agent | Name | | Application |
| No. | Tradename | Compound Structure | Publications |
| A1 | Sotrastaurin | 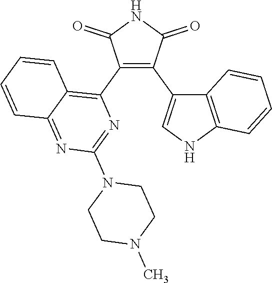 [SMILES: CN1CCN(c2nc(C3=C(c4c[nH]c5ccccc45)C(=O)NC3=O)c3ccccc3n2)CC1] | EP 1682103 US 2007/142401 WO 2005/039549 |
| A2 | Nilotinib HCl monohydrate TASIGNA ® | 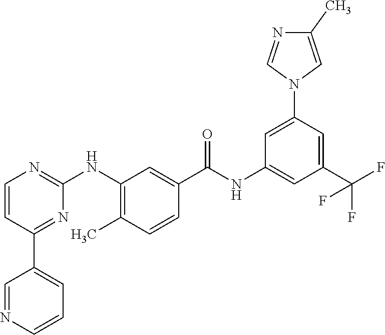 [SMILES: Cc1cn(-c2cc(NC(=O)c3ccc(C)c(Nc4nccc(-c5cccnc5)n4)c3)cc(C(F)(F)F)c2)cn1] | WO 2004/005281 U.S. Pat. No. 7,169,791 |
| | | HCl•H₂O | |
| A3 | | 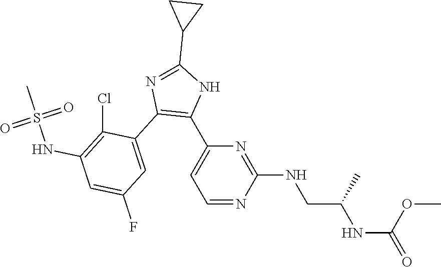 [SMILES: COC(=O)N[C@@H](C)CNc1nccc(-c2[nH]c(C3CC3)nc2-c2cc(F)cc(NS(C)(=O)=O)c2Cl)n1] | WO2011/023773 |
| A4 | | 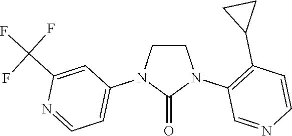 [SMILES: O=C1N(c2ccnc(C(F)(F)F)c2)CCN1c1cnccc1C1CC1] | WO2012/149413 |
| A6 | | 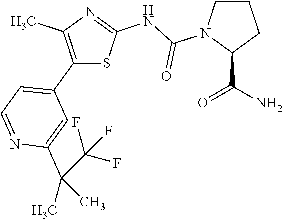 [SMILES: Cc1nc(NC(=O)N2CCC[C@H]2C(N)=O)sc1-c1ccnc(C(C)(C)C(F)(F)F)c1] | WO 2010/029082 |
| A7 | | 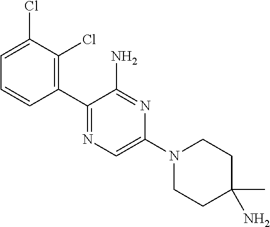 [SMILES: CC1(N)CCN(c2cnc(-c3cccc(Cl)c3Cl)c(N)n2)CC1] | WO2015/107493 |
| A8 | | | WO2015/107495 |
| A9 | | 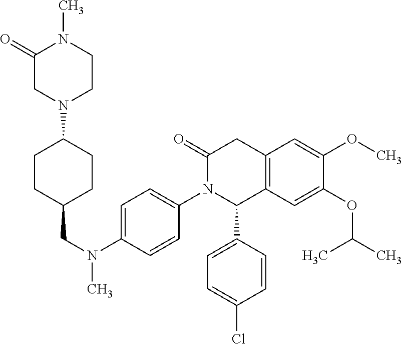 [SMILES: COc1cc2c(cc1OC(C)C)[C@H](c1ccc(Cl)cc1)N(c1ccc(N(C)C[C@H]3CC[C@H](N4CCN(C)C(=O)C4)CC3)cc1)C(=O)C2] | WO 2011/076786 |
| A10 | Deferasirox EXJADE ® | 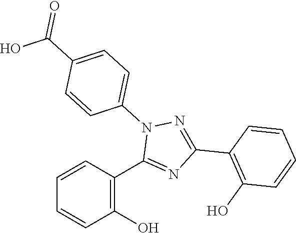 [SMILES: O=C(O)c1ccc(-n2nc(-c3ccccc3O)nc2-c2ccccc2O)cc1] | WO 1997/049395 |
| A11 | Letrozole FEMARA ® | 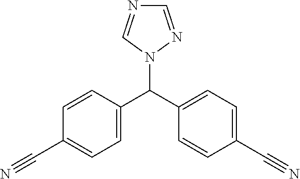 [SMILES: N#Cc1ccc(C(c2ccc(C#N)cc2)n2cncn2)cc1] | U.S. Pat. No. 4,978,672 |
| A12 | | 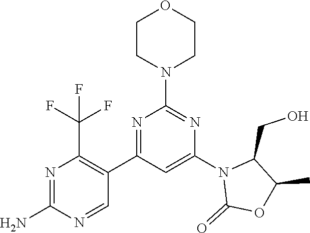 [SMILES: C[C@H]1OC(=O)N(c2cc(-c3cnc(N)nc3C(F)(F)F)nc(N3CCOCC3)n2)[C@H]1CO] | WO 2013/124826 US 2013/0225574 |
| A13 | | 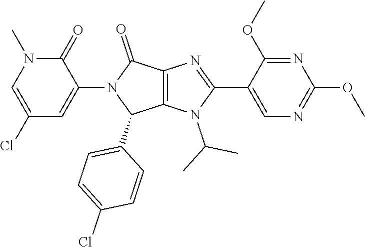 [SMILES: COc1ncc(-c2nc3c(n2C(C)C)[C@H](c2ccc(Cl)cc2)N(c2cc(Cl)cn(C)c2=O)C3=O)c(OC)n1] | WO 2013/111105 |
| A14 | | 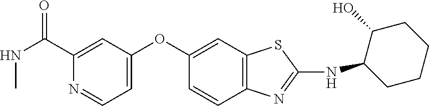 [SMILES: CNC(=O)c1cc(Oc2ccc3nc(N[C@@H]4CCCC[C@H]4O)sc3c2)ccn1] | WO2007/121484 |
| A15 | Imatinib mesylate GLEEVEC ® | 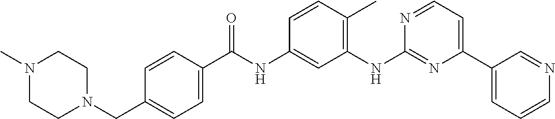 [SMILES: Cc1ccc(NC(=O)c2ccc(CN3CCN(C)CC3)cc2)cc1Nc1nccc(-c2cccnc2)n1] | WO 1999/003854 |
| | | Mesylate | |
| A16 | Capmatinib | 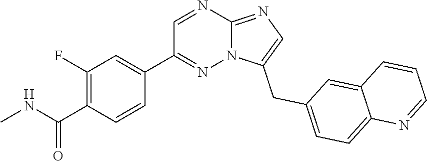 [SMILES: CNC(=O)c1ccc(-c2cnc3ncc(Cc4ccc5ncccc5c4)n3n2)cc1F] | EP 2099447 U.S. Pat. No. 7,767,675 U.S. Pat. No. 8,420,645 |
| | | Dihydrochloric salt | |
| A17 | Ruxolitinib Phosphate JAKAFI ® | 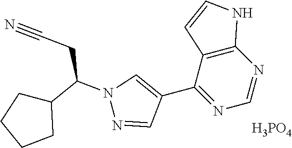 | WO 2007/070514 EP 2474545 U.S. Pat. No. 7,598,257 WO 2014/018632 |
| A18 | Panobinostat | 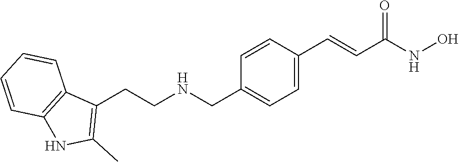 [SMILES: Cc1[nH]c2ccccc2c1CCNCc1ccc(/C=C/C(=O)NO)cc1] | WO 2014/072493 WO 2002/022577 EP 1870399 |
| A20 | | 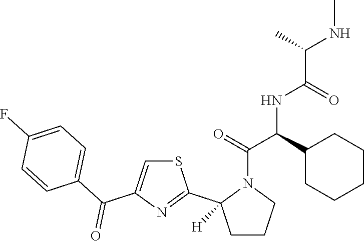 [SMILES: CN[C@@H](C)C(=O)N[C@H](C(=O)N1CCC[C@H]1c1nc(C(=O)c2ccc(F)cc2)cs1)C1CCCCC1] | WO 2008/016893 EP 2051990 U.S. Pat. No. 8,552,003 |
| A21 | | 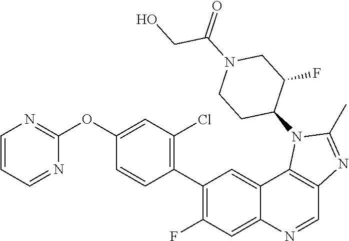 [SMILES: Cc1nc2cnc3cc(F)c(-c4ccc(Oc5ncccn5)cc4Cl)cc3c2n1[C@H]1CCN(C(=O)CO)C[C@@H]1F] | WO2015/022662 |
| A22 | ceritinib ZYKADIA ™ | 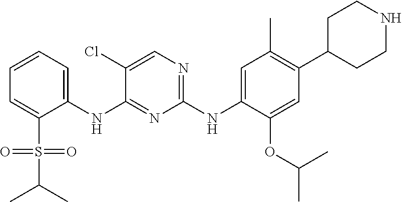 [SMILES: Cc1cc(Nc2ncc(Cl)c(Nc3ccccc3S(=O)(=O)C(C)C)n2)c(OC(C)C)cc1C1CCNCC1] | WO 2008/073687 U.S. Pat. No. 8,039,479 |
| A23 | Ribociclib KISQALI ® | 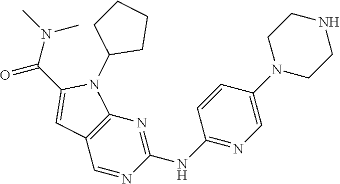 [SMILES: CN(C)C(=O)c1cc2cnc(Nc3ccc(N4CCNCC4)cn3)nc2n1C1CCCC1] | U.S. Pat. No. 8,415,355 U.S. Pat. No. 8,685,980 |
| A24 | | 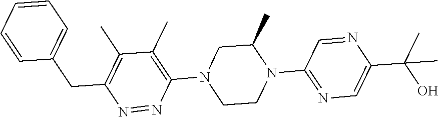 [SMILES: Cc1c(Cc2ccccc2)nnc(N2CCN(c3cnc(C(C)(C)O)cn3)[C@H](C)C2)c1C] | WO 2010/007120 |
| A26 | | 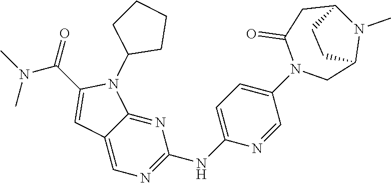 | WO 2011/101409 |
| A27 | | Human monoclonal antibody to HER3 | WO 2012/022814 |
| | | | EP 2606070 |
| | | | U.S. Pat. No. 8,735,551 |
| A28 | | Antibody Drug Conjugate (ADC) | WO 2014/160160 |
| A29 | | Monoclonal antibody or Fab to M-CSF | WO 2004/045532 |
| A30 | Midostaurin | 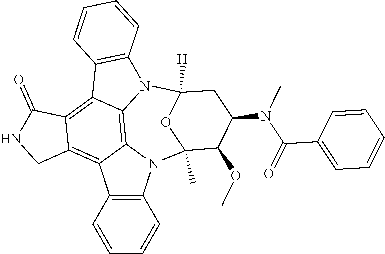 [SMILES: CO[C@@H]1[C@H](N(C)C(=O)c2ccccc2)C[C@H]2O[C@]1(C)n1c3ccccc3c3c4c(c5c6ccccc6n2c5c31)C(=O)NC4] | WO 2003/037347; EP 1441737 US 2012/252785 |
| A31 | Everolimus AFINITOR ® | 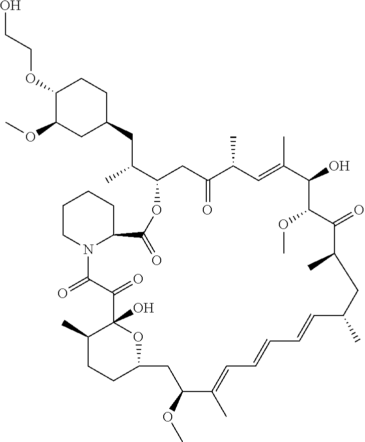 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCO)[C@H](OC)C2)CC(=O)[C@H](C)/C=C(\C)[C@@H](O)[C@@H](OC)C(=O)[C@H](C)C[C@H](C)/C=C/C=C/C=C/1C] | WO 1994/009010 WO 2014/085318 |
| A32 | | 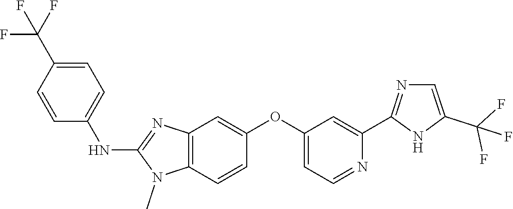 [SMILES: Cn1c(Nc2ccc(C(F)(F)F)cc2)nc2cc(Oc3ccnc(-c4ncc(C(F)(F)F)[nH]4)c3)ccc21] | WO 2007/030377 U.S. Pat. No. 7,482,367 |
| A34 | | 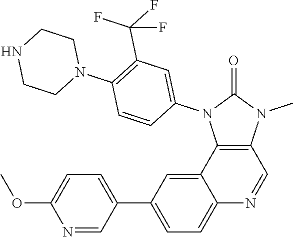 | WO 2006/122806 |
| A35 | | 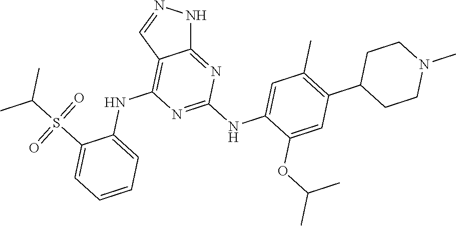 [SMILES: Cc1cc(Nc2nc(Nc3ccccc3S(=O)(=O)C(C)C)c3cn[nH]c3n2)c(OC(C)C)cc1C1CCN(C)CC1] | WO 2008/073687 U.S. Pat. No. 8,372,858 |
| A36 | Valspodar AMDRAY ™ | 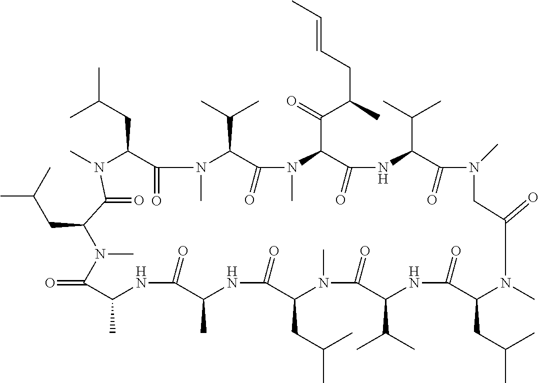 [SMILES: C/C=C/C[C@@H](C)C(=O)[C@H]1C(=O)N[C@@H](C(C)C)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C] | EP 296122 |
| A37 | Vatalanib succinate | 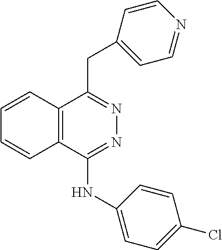 [SMILES: Clc1ccc(Nc2nnc(Cc3ccncc3)c3ccccc23)cc1] | WO 98/35958 |
| | | succinate | |
| A38 | | 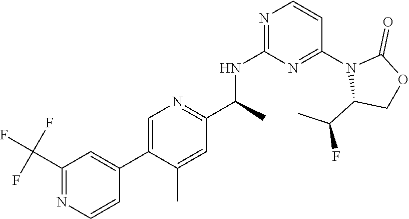 [SMILES: Cc1cc([C@H](C)Nc2nccc(N3C(=O)OC[C@@H]3[C@H](C)F)n2)ncc1-c1ccnc(C(F)(F)F)c1] | WO2014/141104 |
| A39 | Asciminib | 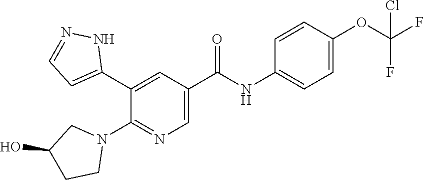 [SMILES: O=C(Nc1ccc(OC(F)(F)Cl)cc1)c1cnc(N2CC[C@@H](O)C2)c(-c2ccn[nH]2)c1] | WO2013/171639 WO2013/171640 WO2013/171641 WO2013/171642 |
| A42 | | 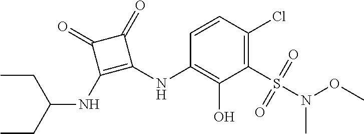 [SMILES: CCC(CC)Nc1c(Nc2ccc(Cl)c(S(=O)(=O)N(C)OC)c2O)c(=O)c1=O] | WO2010/015613 WO2013030803 U.S. Pat. No. 7,989,497, |
| | | or a choline salt thereof | |
| A43 | | | WO 2017/025918 |
| | | | WO2011/121418 |
| | | | U.S. Pat. No. 8,796,284 |
| A44 | | 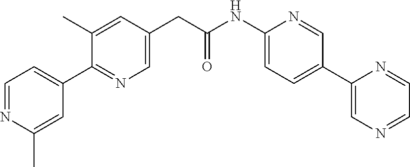 [SMILES: Cc1cc(-c2ncc(CC(=O)Nc3ccc(-c4cnccn4)cn3)cc2C)ccn1] | WO2010/101849 |
| A45 | | 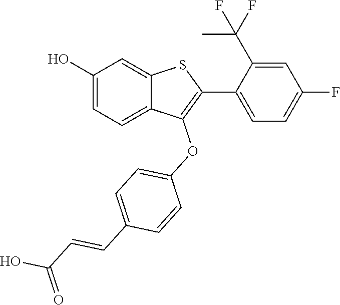 [SMILES: CC(F)(F)c1cc(F)ccc1-c1sc2cc(O)ccc2c1Oc1ccc(/C=C/C(=O)O)cc1] | WO2014/130310 |
| A46 | trametinib | 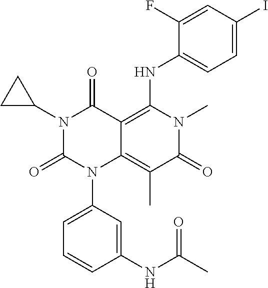 [SMILES: CC(=O)Nc1cccc(-n2c(=O)n(C3CC3)c(=O)c3c(Nc4ccc(I)cc4F)n(C)c(=O)c(C)c32)c1] | WO2005/121142 U.S. Pat. No. 7,378,423 |
| A47 | dabrafenib | 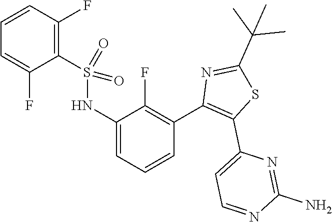 [SMILES: CC(C)(C)c1nc(-c2cccc(NS(=O)(=O)c3c(F)cccc3F)c2F)c(-c2ccnc(N)n2)s1] | WO 2009/137391 U.S. Pat. No. 7,994,185 |
| A49 | octreotide | 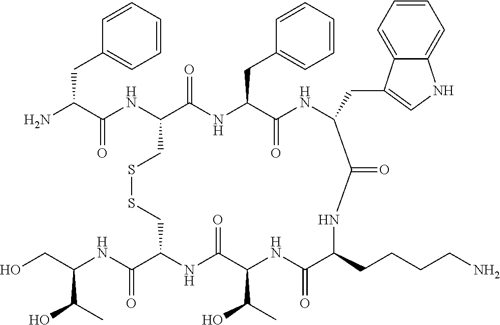 [SMILES: C[C@@H](O)[C@@H]1NC(=O)[C@H](CCCCN)NC(=O)[C@@H](Cc2c[nH]c3ccccc23)NC(=O)[C@H](Cc2ccccc2)NC(=O)[C@@H](NC(=O)[C@H](N)Cc2ccccc2)CSSC[C@@H](C(=O)N[C@H](CO)[C@@H](C)O)NC1=O] | U.S. Pat. No. 4,395,403 EP 0 029 579 |
| A50 | | 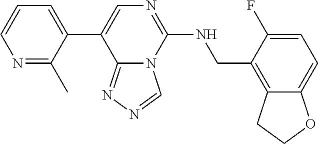 [SMILES: Cc1ncccc1-c1cnc(NCc2c(F)ccc3c2CCO3)n2cnnc12] | WO 2016/103155 U.S. Pat. No. 9,580,437 EP 3237418 |
| A51 | | 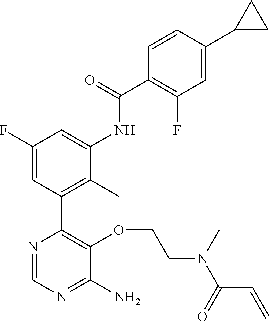 [SMILES: C=CC(=O)N(C)CCOc1c(N)ncnc1-c1cc(F)cc(NC(=O)c2ccc(C3CC3)cc2F)c1C] | U.S. Pat. No. 9,512,084 WO/2015/079417 |
| A52 | | 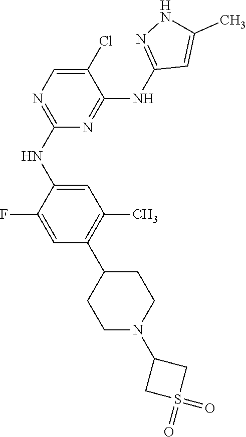 [SMILES: Cc1cc(Nc2nc(Nc3cc(C)c(C4CCN(C5CS(=O)(=O)C5)CC4)cc3F)ncc2Cl)n[nH]1] | WO 2010/002655 U.S. Pat. No. 8,519,129 |
| A53 | | 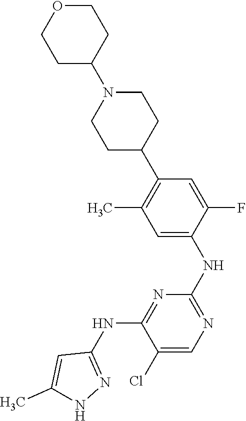 [SMILES: Cc1cc(Nc2nc(Nc3cc(C)c(C4CCN(C5CCOCC5)CC4)cc3F)ncc2Cl)n[nH]1] | WO 2010/002655 U.S. Pat. No. 8,519,129 |
| A54 | | 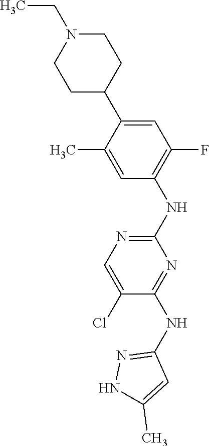 [SMILES: CCN1CCC(c2cc(F)c(Nc3ncc(Cl)c(Nc4cc(C)[nH]n4)n3)cc2C)CC1] | WO 2010/002655 |

### Estrogen Receptor Antagonists

In some embodiments, an estrogen receptor (ER) antagonist is used in combination with the compounds of Formula (I) or compounds of Embodiment 16, 17, or 35, or a pharmaceutically acceptable salt, hydrate, solvate, prodrug, stereoisomer, or tautomer thereof, for treating a disease, e.g., cancer. In some embodiments, the estrogen receptor antagonist is a selective estrogen receptor degrader (SERD). SERDs are estrogen receptor antagonists which bind to the receptor and result in e.g., degradation or down-regulation of the receptor (Boer K. et al., (2017) Therapeutic Advances in Medical Oncology 9(7): 465-479). ER is a hormone-activated transcription factor important for e.g., the growth, development and physiology of the human reproductive system. ER is activated by, e.g., the hormone estrogen (17beta estradiol). ER expression and signaling is implicated in cancers (e.g., breast cancer), e.g., ER positive (ER+) breast cancer. In some embodiments, the SERD is chosen from LSZ102, fulvestrant, brilanestrant, or elacestrant.

### Exemplary Estrogen Receptor Antagonists

In some embodiments, the SERD comprises a compound disclosed in International Application Publication No. WO 2014/130310, which is hereby incorporated by reference in its entirety. In some embodiments, the SERD comprises LSZ102. LSZ102 has the chemical name: (E)-3-(4-((2-(2-(1,1-difluoroethyl)-4-fluorophenyl)-6-hydroxybenzo[b]thiophen-3-yl)oxy)phenyl)acrylic acid.

### Other Exemplary Estrogen Receptor Antagonists

In some embodiments, the SERD comprises fulvestrant (CAS Registry Number: 129453-61-8), or a compound disclosed in International Application Publication No. WO 2001/051056, which is hereby incorporated by reference in its entirety. Fulvestrant is also known as ICI 182780, ZM 182780, FASLODEX®, or (7α,17β)-7-{9-[(4,4,5,5,5-pentafluoropentyl)sulfinyl]nonyl}estra-1,3,5(10)-triene-3,17-diol. Fulvestrant is a high affinity estrogen receptor antagonist with an IC50 of 0.29 nM.

In some embodiments, the SERD comprises elacestrant (CAS Registry Number: 722533-56-4), or a compound disclosed in U.S. Pat. No. 7,612,114, which is incorporated by reference in its entirety. Elacestrant is also known as RAD1901, ER-306323 or (6R)-6-{2-[Ethyl({4-[2-(ethylamino)ethyl]phenyl}methyl)amino]-4-methoxyphenyl}-5,6,7,8-tetrahydronaphthalen-2-ol. Elacestrant is an orally bioavailable, non-steroidal combined selective estrogens receptor modulator (SERM) and a SERD. Elacestrant is also disclosed, e.g., in Garner F et al., (2015) Anticancer Drugs 26(9):948-56.

In some embodiments, the SERD is brilanestrant (CAS Registry Number: 1365888-06-7), or a compound disclosed in International Application Publication No. WO 2015/136017, which is incorporated by reference in its entirety. Brilanestrant is also known as GDC-0810, ARN810, RG-6046, RO-7056118 or (2E)-3-{4-[(1E)-2-(2-chloro-4-fluorophenyl)-1-(1H-indazol-5-yl)but-1-en-1-yl]phenyl}prop-2-enoic acid. Brilanestrant is a next-generation, orally bioavailable selective SERD with an IC50 of 0.7 nM. Brilanestrant is also disclosed, e.g., in Lai A. et al. (2015) Journal of Medicinal Chemistry 58 (12): 4888-4904.

In some embodiments, the SERD is chosen from RU 58668, GW7604, AZD9496, bazedoxifene, pipendoxifene, arzoxifene, OP-1074, or acolbifene, e.g., as disclosed in McDonell et al. (2015) Journal of Medicinal Chemistry 58(12) 4883-4887. Other exemplary estrogen receptor antagonists are disclosed, e.g., in WO 2011/156518, WO 2011/159769, WO 2012/037410, WO 2012/037411, and US 2012/0071535, all of which are hereby incorporated by reference in their entirety.

### CDK4/6 Inhibitors

In some embodiments, an inhibitor of Cyclin-Dependent Kinases 4 or 6 (CDK4/6) is used in combination with the compounds of Formula (I) or compounds of Embodiment 16, 17, or 35, or a pharmaceutically acceptable salt, hydrate, solvate, prodrug, stereoisomer, or tautomer thereof, for treating a disease, e.g., cancer. In some embodiments, the CDK4/6 inhibitor is chosen from ribociclib, abemaciclib (Eli Lilly), or palbociclib.

### Exemplary CDK4/6 Inhibitors

In some embodiments, the CDK4/6 inhibitor comprises ribociclib (CAS Registry Number: 1211441-98-3), or a compound disclosed in U.S. Pat. Nos. 8,415,355 and 8,685,980, which are incorporated by reference in their entirety.

In some embodiments, the CDK4/6 inhibitor comprises a compound disclosed in International Application Publication No. WO 2010/020675 and U.S. Pat. Nos. 8,415,355 and 8,685,980, which are incorporated by reference in their entirety.

In some embodiments, the CDK4/6 inhibitor comprises ribociclib (CAS Registry Number: 1211441-98-3). Ribociclib is also known as LEE011, KISQALI®, or 7-cyclopentyl-N,N-dimethyl-2-((5-(piperazin-1-yl)pyridin-2-yl)amino)-7H-pyrrolo[2,3-d]pyrimidine-6-carboxamide.

### Other Exemplary CDK4/6 Inhibitors

In some embodiments, the CDK4/6 inhibitor comprises abemaciclib (CAS Registry Number: 1231929-97-7). Abemaciclib is also known as LY835219 or N-[5-[(4-Ethyl-1-piperazinyl)methyl]-2-pyridinyl]-5-fluoro-4-[4-fluoro-2-methyl-1-(1-methylethyl)-1H-benzimidazol-6-yl]-2-pyrimidinamine. Abemaciclib is a CDK inhibitor selective for CDK4 and CDK6 and is disclosed, e.g., in Torres-Guzman R et al. (2017) Oncotarget 10.18632/oncotarget.17778.

In some embodiments, the CDK4/6 inhibitor comprises palbociclib (CAS Registry Number: 571190-30-2). Palbociclib is also known as PD-0332991, IBRANCE® or 6-Acetyl-8-cyclopentyl-5-methyl-2-{[5-(1-piperazinyl)-2-pyridinyl]amino}pyrido[2,3-d]pyrimidin-7(8H)-one. Palbociclib inhibits CDK4 with an IC50 of 1 nM, and inhibits CDK6 with an IC50 of 16 nM, and is disclosed, e.g., in Finn et al. (2009) Breast Cancer Research 11(5):R77.

### CXCR2 Inhibitors

In some embodiments, an inhibitor of chemokine (C-X-C motif) receptor 2 (CXCR2) is used in combination with the compounds of Formula (I) or compounds of Embodiment 16, 17, or 35, or a pharmaceutically acceptable salt, hydrate, solvate, prodrug, stereoisomer, or tautomer thereof, for treating a disease, e.g., cancer. In some embodiments, the CXCR2 inhibitor is chosen from 6-chloro-3-((3,4-dioxo-2-(pentan-3-ylamino)cyclobut-1-en-1-yl)amino)-2-hydroxy-N-methoxy-N-methylbenzenesulfonamide, danirixin, reparixin, or navarixin.

### Exemplary CXCR2 Inhibitors

In some embodiments, the CXCR2 inhibitor comprises a compound disclosed in U.S. Pat. Nos. 7,989,497, 8,288,588, 8,329,754, 8,722,925, 9,115,087, U.S. Application Publication Nos. US 2010/0152205, US 2011/0251205 and US 2011/0251206, and International Application Publication Nos. WO 2008/061740, WO 2008/061741, WO 2008/062026, WO 2009/106539, WO2010/063802, WO 2012/062713, WO 2013/168108, WO 2010/015613 and WO 2013/030803. In some embodiments, the CXCR2 inhibitor comprises 6-chloro-3-((3,4-dioxo-2-(pentan-3-ylamino)cyclobut-1-en-1-yl)amino)-2-hydroxy-N-methoxy-N-methylbenzenesulfonamide or a choline salt thereof. In some embodiments, the CXCR2 inhibitor comprises 6-chloro-3-((3,4-dioxo-2-(pentan-3-ylamino)cyclobut-1-en-1-yl)amino)-2-hydroxy-N-methoxy-N-methylbenzenesulfonamide choline salt. In some embodiments, the CXCR2 inhibitor is 2-Hydroxy-N,N,N-trimethylethan-1-aminium 3-chloro-6-({3,4-dioxo-2-[(pentan-3-yl)amino]cyclobut-1-en-1-yl}amino)-2-(N-methoxy-N-methylsulfamoyl)phenolate (i.e., 6-chloro-3-((3,4-dioxo-2-(pentan-3-ylamino)cyclobut-1-en-1-yl)amino)-2-hydroxy-N-methoxy-N-methylbenzenesulfonamide choline salt) and has the following chemical structure:

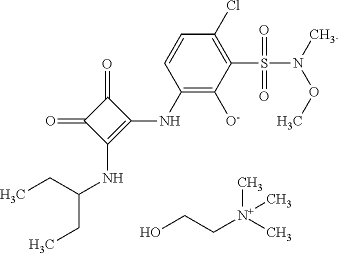

### Other Exemplary CXCR2Inhibitors

In some embodiments, the CXCR2 inhibitor comprises danirixin (CAS Registry Number: 954126-98-8). Danirixin is also known as GSK1325756 or 1-(4-chloro-2-hydroxy-3-piperidin-3-ylsulfonylphenyl)-3-(3-fluoro-2-methylphenyl)urea. Danirixin is disclosed, e.g., in Miller et al. Eur J Drug Metab Pharmacokinet (2014) 39:173-181; and Miller et al. BMC Pharmacology and Toxicology (2015), 16:18.

In some embodiments, the CXCR2 inhibitor comprises reparixin (CAS Registry Number: 266359-83-5). Reparixin is also known as repertaxin or (2R)-2-[4-(2-methylpropyl)phenyl]-N-methylsulfonylpropanamide. Reparixin is a non-competitive allosteric inhibitor of CXCR1/2.

Reparixin is disclosed, e.g., in Zarbock et al. Br J Pharmacol. 2008; 155(3):357-64.

In some embodiments, the CXCR2 inhibitor comprises navarixin. Navarixin is also known as MK-7123, SCH 527123, PS291822, or 2-hydroxy-N,N-dimethyl-3-[[2-[[(1R)-1-(5-methylfuran-2-yl)propyl]amino]-3,4-dioxocyclobuten-1-yl]amino]benzamide. Navarixin is disclosed, e.g., in Ning et al. Mol Cancer Ther. 2012; 11(6):1353-64.

### CSF-1/1JR Binding Agents

In some embodiments, a CSF-1/1R binding agent is used in combination with the compounds of Formula (I) or compounds of Embodiment 16, 17, or 35, or a pharmaceutically acceptable salt, hydrate, solvate, prodrug, stereoisomer, or tautomer thereof, for treating a disease, e.g., cancer. In some embodiments, the CSF-1/1R binding agent is chosen from an inhibitor of macrophage colony-stimulating factor (M-CSF), e.g., a monoclonal antibody or Fab to M-CSF (e.g., MCS110), a CSF-1R tyrosine kinase inhibitor (e.g., 4-((2-(((1R,2R)-2-hydroxycyclohexyl)amino)benzo[d]thiazol-6-yl)oxy)-N-methylpicolinamide or BLZ945), a receptor tyrosine kinase inhibitor (RTK) (e.g., pexidartinib), or an antibody targeting CSF-1R (e.g., emactuzumab or FPA008). In some embodiments, the CSF-1/1R inhibitor is BLZ945. In some embodiments, the CSF-1/1R binding agent is MCS110. In other embodiments, the CSF-1/1R binding agent is pexidartinib.

### Exemplary CSF-1 Binding Agents

In some embodiments, the CSF-1/1R binding agent comprises an inhibitor of macrophage colony-stimulating factor (M-CSF). M-CSF is also sometimes known as CSF-1. In certain embodiments, the CSF-1/1R binding agent is an antibody to CSF-1 (e.g., MCS110). In other embodiments, the CSF-1/1R binding agent is an inhibitor of CSF-1R (e.g., BLZ945).

In some embodiments, the CSF-1/1R binding agent comprises a monoclonal antibody or Fab to M-CSF (e.g., MCS110/H-RX1), or a binding agent to CSF-1 disclosed in International Application Publication Nos. WO 2004/045532 and WO 2005/068503, including H-RX1 or 5H4 (e.g., an antibody molecule or Fab fragment against M-CSF) and U.S. Pat. No. 9,079,956, which applications and patent are incorporated by reference in their entirety.

TABLE 13a
Amino acid and nucleotide sequences
of an exemplary anti-M-CSF antibody
molecule (MCS110)

| | |
|---|---|
| (H-RX1)HC | QVQLQESGPGLVRPSQTLSL |
| | TCTVSDYSITSDYAWNWIRQ |
| | FPGRGLEWMGYISYSGSTSY |
| | NPSLRSRITISRDTSRNQFS |
| | LQLNSVTAADTAVYYCASFD |
| | YAHAMDYVVGQGTTVTVSSA |
| | STRGPSVFPLAPSSRSTSGG |
| | TAALGCLVRDYFPEPVTVSW |
| | NSGALTSGVHTFPAVLQSSG |
| | LYSLSSVVTVPSSSLGTQTY |
| | ICNVNHRPSNTRVDRRVEPR |
| | SCDRTHTCPPCPAPELLGGP |
| | SVFLFPPRPRDTLMISRTPE |
| | VTCVVVDVSHEDPEVRFNWY |
| | VDGVEVHNARTRPREEQYNS |
| | TYRVVSVLTVLHQDWLNGRE |
| | YRCRVSNRALPAPIERTISR |
| | ARGQPREPQVYTLPPSREEM |
| | TRNQVSLTCLVRGFYPSDIA |
| | VEWESNGQPENNYRTTPPVL |
| | DSDGSFFLYSRLTVDRSRWQ |
| | QGNVFSCSVMHEALHNHYTQ |
| | RSLSLSPGR |
| | (SEQ ID NO: 271) |
| (H-RX1)LC | DIVLTQSPAFLSVTPGERVT |
| | FTCQASQSIGTSIHWYQQRT |
| | DQAPRLLIRYASESISGIPS |
| | RFSGSGSGTDFTLTISSVEA |
| | EDAADYYCQQINSWPTTFGG |
| | GTRLEIRRTVAAPSVFIFPP |
| | SDEQLRSGTASVVCLLNNFY |
| | PREARVQWRVDNALQSGNSQ |
| | ESVTEQDSRDSTYSLSSTLT |
| | LSRADYERHRVYACEVTHQG |
| | LSSPVTRSFNRGEC |
| | (SEQ ID NO: 272) |
| Heavy Chain | SDYAWN |
| CDR1 (Rabat) | (SEQ ID NO: 273) |
| Heavy Chain | YISYSGSTSYNPSLRS |
| CDR2 (Rabat) | (SEQ ID NO: 274) |
| Heavy Chain | FDYAHAMDY |
| CDR3 (Rabat) | (SEQ ID NO: 275) |
| Light Chain | QASQSIGTSIH |
| CDR1 (Rabat) | (SEQ ID NO: 276) |
| Light Chain | YASESIS |
| CDR2 (Rabat) | (SEQ ID NO: 277) |
| Light Chain | QQINSWPTT |
| CDR3 (Rabat) | (SEQ ID NO: 278) |

In another embodiment, the CSF-1/1R binding agent comprises a CSF-1R tyrosine kinase inhibitor, 4-((2-(((1R,2R)-2-hydroxycyclohexyl)amino)benzo[d]thiazol-6-yl)oxy)-N-methylpicolinamide (BLZ945), or a compound disclosed in International Application Publication No. WO 2007/121484, and U.S. Pat. Nos. 7,553,854, 8,173,689, and 8,710,048, which are incorporated by reference in their entirety.

### Other Exemplary CSF-1/1R Binding Agents

In some embodiments, the CSF-1/1R binding agent comprises pexidartinib (CAS Registry Number 1029044-16-3). Pexidrtinib is also known as PLX3397 or 5-((5-chloro-1H-pyrrolo[2,3-b]pyridin-3-yl)methyl)-N-((6-(trifluoromethyl)pyridin-3-yl)methyl)pyridin-2-amine. Pexidartinib is a small-molecule receptor tyrosine kinase (RTK) inhibitor of KIT, CSF1R and FLT3. FLT3, CSF1R and FLT3 are overexpressed or mutated in many cancer cell types and play major roles in tumor cell proliferation and metastasis. PLX3397 can bind to and inhibit phosphorylation of stem cell factor receptor (KIT), colony-stimulating factor-i receptor (CSF1R) and FMS-like tyrosine kinase 3 (FLT3), which may result in the inhibition of tumor cell proliferation and down-modulation of macrophages, osteoclasts and mast cells involved in the osteolytic metastatic disease.

In some embodiments, the CSF-1/1R binding agent is emactuzumab. Emactuzumab is also known as RG7155 or R05509554. Emactuzumab is a humanized IgG1 mAb targeting CSF1R. In some embodiments, the CSF-1/1R binding agent is FPA008. FPA008 is a humanized mAb that inhibits CSF1R.

### A2aR Antagonists

In some embodiments, an adenosine A2a receptor (A2aR) antagonist (e.g., an inhibitor of A2aR pathway, e.g., an adenosine inhibitor, e.g., an inhibitor of A2aR or CD-73) is used in combination with the compounds of Formula (I) or compounds of Embodiment 16, 17, or 35, or a pharmaceutically acceptable salt, hydrate, solvate, prodrug, stereoisomer, or tautomer thereof, for treating a disease, e.g., cancer. In some embodiments, the A2aR antagonist is selected from PBF509 (NIR178) (Palobiofarma/Novartis), CPI444/V81444 (Corvus/Genentech), AZD4635/HTL-1071 (AstraZeneca/Heptares), Vipadenant (Redox/Juno), GBV-2034 (Globavir), AB928 (Arcus Biosciences), Theophylline, Istradefylline (Kyowa Hakko Kogyo), Tozadenant/SYN-115 (Acorda), KW-6356 (Kyowa Hakko Kogyo), ST-4206 (Leadiant Biosciences), and Preladenant/SCH 420814 (Merck/Schering).

### Exemplary A2aR Antagonists

In some embodiments, the A2aR antagonist comprises PBF509 (NIR178) or a compound disclosed in U.S. Pat. No. 8,796,284 or in International Application Publication No. WO 2017/025918, herein incorporated by reference in their entirety. PBF509 (NIR178) is also known as NIR178.

### Other Exemplary A2aR Antagonists

In certain embodiments, the A2aR antagonist comprises CPI444/V81444. CPI-444 and other A2aR antagonists are disclosed in International Application Publication No. WO 2009/156737, herein incorporated by reference in its entirety. In certain embodiments, the A2aR antagonist is (S)-7-(5-methylfuran-2-yl)-3-((6-(((tetrahydrofuran-3-yl)oxy)methyl)pyridin-2-yl)methyl)-3H-[1,2,3]triazolo[4,5-d]pyrimidin-5-amine. In certain embodiments, the A2aR antagonist is (R)-7-(5-methylfuran-2-yl)-3-((6-(((tetrahydrofuran-3-yl)oxy)methyl)pyridin-2-yl)methyl)-3H-[1,2,3]triazolo[4,5-d]pyrimidin-5-amine, or racemate thereof. In certain embodiments, the A2aR antagonist is 7-(5-methylfuran-2-yl)-3-((6-(((tetrahydrofuran-3-yl)oxy)methyl)pyridin-2-yl)methyl)-3H-[1,2,3]triazolo[4,5-d]pyrimidin-5-amine.

In certain embodiments, the A2aR antagonist is AZD4635/HTL-1071. A2aR antagonists are disclosed in International Application Publication No. WO 2011/095625, herein incorporated by reference in its entirety. In certain embodiments, the A2aR antagonist is 6-(2-chloro-6-methylpyridin-4-yl)-5-(4-fluorophenyl)-1,2,4-triazin-3-amine.

In certain embodiments, the A2aR antagonist is ST-4206 (Leadiant Biosciences). In certain embodiments, the A2aR antagonist is an A2aR antagonist described in U.S. Pat. No. 9,133,197, herein incorporated by reference in its entirety.

In certain embodiments, the A2aR antagonist is an A2aR antagonist described in U.S. Pat. Nos. 8,114,845 and 9,029,393, U.S. Application Publication Nos. 2017/0015758 and 2016/0129108, herein incorporated by reference in their entirety.

In some embodiments, the A2aR antagonist is istradefylline (CAS Registry Number: 155270-99-8). Istradefylline is also known as KW-6002 or 8-[(E)-2-(3,4-dimethoxyphenyl)vinyl]-1,3-diethyl-7-methyl-3,7-dihydro-TH-purine-2,6-dione. Istradefylline is disclosed, e.g., in LeWitt et al. (2008) Annals of Neurology 63 (3): 295-302).

In some embodiments, the A2aR antagonist is tozadenant (Biotie). Tozadenant is also known as SYN115 or 4-hydroxy-N-(4-methoxy-7-morpholin-4-yl-1,3-benzothiazol-2-yl)-4-methylpiperidine-1-carboxamide. Tozadenant blocks the effect of endogenous adenosine at the A2a receptors, resulting in the potentiation of the effect of dopamine at the D2 receptor and inhibition of the effect of glutamate at the mGluR5 receptor. In some embodiments, the A2aR antagonist is preladenant (CAS Registry Number: 377727-87-2). Preladenant is also known as SCH 420814 or 2-(2-Furanyl)-7-[2-[4-[4-(2-methoxyethoxy)phenyl]-1-piperazinyl]ethyl]7H-pyrazolo[4,3-e][1,2,4]triazolo[1,5-c]pyrimidine-5-amine. Preladenant was developed as a drug that acted as a potent and selective antagonist at the adenosine A2A receptor.

In some embodiments, the A2aR antagonist is vipadenan. Vipadenan is also known as BIIB014, V2006, or 3-[(4-amino-3-methylphenyl)methyl]-7-(furan-2-yl)triazolo[4,5-d]pyrimidin-5-amine. Other exemplary A2aR antagonists include, e.g., ATL-444, MSX-3, SCH-58261, SCH-412,348, SCH-442,416, VER-6623, VER-6947, VER-7835, CGS-15943, and ZM-241,385.

In some embodiments, the A2aR antagonist is an A2aR pathway antagonist (e.g., a CD-73 inhibitor, e.g., an anti-CD73 antibody) is MEDI9447. MEDI9447 is a monoclonal antibody specific for CD73. Targeting the extracellular production of adenosine by CD73 may reduce the immunosuppressive effects of adenosine. MEDI9447 was reported to have a range of activities, e.g., inhibition of CD73 ectonucleotidase activity, relief from AMP-mediated lymphocyte suppression, and inhibition of syngeneic tumor growth. MEDI9447 can drive changes in both myeloid and lymphoid infiltrating leukocyte populations within the tumor microenvironment.

These changes include, e.g., increases in CD8 effector cells and activated macrophages, as well as a reduction in the proportions of myeloid-derived suppressor cells (MDSC) and regulatory T lymphocytes.

### IDO Inhibitors

In some embodiments, an inhibitor of indoleamine 2,3-dioxygenase (IDO) and/or tryptophan 2,3-dioxygenase (TDO) is used in combination with the compounds of Formula (I) or compounds of Embodiment 16, 17, or 35, or a pharmaceutically acceptable salt, hydrate, solvate, prodrug, stereoisomer, or tautomer thereof, for treating a disease, e.g., cancer. In some embodiments, the IDO inhibitor is chosen from (4E)-4-[(3-chloro-4-fluoroanilino)-nitrosomethylidene]-1,2,5-oxadiazol-3-amine (also known as epacadostat or INCB24360), indoximod ( ), (1-methyl-D-tryptophan), α-cyclohexyl-5H-Imidazo[5,1-a]isoindole-5-ethanol (also known as NLG919), indoximod, and BMS-986205 (formerly F001287).

### Exemplary IDO Inhibitors

In some embodiments, the IDO/TDO inhibitor is indoximod (New Link Genetics). Indoximod, the D isomer of 1-methyl-tryptophan, is an orally administered small-molecule indoleamine 2,3-dioxygenase (IDO) pathway inhibitor that disrupts the mechanisms by which tumors evade immune-mediated destruction.

In some embodiments, the IDO/TDO inhibitor is NLG919 (New Link Genetics). NLG919 is a potent IDO (indoleamine-(2,3)-dioxygenase) pathway inhibitor with Ki/EC50 of 7 nM/75 nM in cell-free assays.

In some embodiments, the IDO/TDO inhibitor is epacadostat (CAS Registry Number: 1204669-58-8). Epacadostat is also known as INCB24360 or INCB024360 (Incyte). Epacadostat is a potent and selective indoleamine 2,3-dioxygenase (IDO1) inhibitor with IC50 of 10 nM, highly selective over other related enzymes such as IDO2 or tryptophan 2,3-dioxygenase (TDO).

In some embodiments, the IDO/TDO inhibitor is F001287 (Flexus/BMS). F001287 is a small molecule inhibitor of indoleamine 2,3-dioxygenase 1 (IDO1).

### STING Agonists

In some embodiments, a STING agonist is used in combination with the compounds of Formula (I) or compounds of Embodiment 16, 17, or 35, or a pharmaceutically acceptable salt, hydrate, solvate, prodrug, stereoisomer, or tautomer thereof, for treating a disease, e.g., cancer. In some embodiments, the STING agonist is cyclic dinucleotide, e.g., a cyclic dinucleotide comprising purine or pyrimidine nucleobases (e.g., adenosine, guanine, uracil, thymine, or cytosine nucleobases). In some embodiments, the nucleobases of the cyclic dinucleotide comprise the same nucleobase or different nucleobases.

In some embodiments, the STING agonist comprises an adenosine or a guanosine nucleobase. In some embodiments, the STING agonist comprises one adenosine nucleobase and one guanosine nucleobase. In some embodiments, the STING agonist comprises two adenosine nucleobases or two guanosine nucleobases.

In some embodiments, the STING agonist comprises a modified cyclic dinucleotide, e.g., comprising a modified nucleobase, a modified ribose, or a modified phosphate linkage. In some embodiments, the modified cyclic dinucleotide comprises a modified phosphate linkage, e.g., a thiophosphate.

In some embodiments, the STING agonist comprises a cyclic dinucleotide (e.g., a modified cyclic dinucleotide) with 2′,5′ or 3′,5′ phosphate linkages. In some embodiments, the STING agonist comprises a cyclic dinucleotide (e.g., a modified cyclic dinucleotide) with Rp or Sp stereochemistry around the phosphate linkages.

In some embodiments, the STING agonist is MK-1454 (Merck). MK-1454 is a cyclic dinucleotide Stimulator of Interferon Genes (STING) agonist that activates the STING pathway. Exemplary STING agonist are disclosed, e.g., in PCT Publication No. WO 2017/027645.

### Galectin Inhibitors

In some embodiments, a Galectin, e.g., Galectin-1 or Galectin-3, inhibitor is used in combination with the compounds of Formula (I) or compounds of Embodiment 16, 17, or 35, or a pharmaceutically acceptable salt, hydrate, solvate, prodrug, stereoisomer, or tautomer thereof, for treating a disease, e.g., cancer. In some embodiments, the combination comprises a Galectin-1 inhibitor and a Galectin-3 inhibitor. In some embodiments, the combination comprises a bispecific inhibitor (e.g., a bispecific antibody molecule) targeting both Galectin-1 and Galectin-3. In some embodiments, the Galectin inhibitor is chosen from an anti-Galectin antibody molecule, GR-MD-02 (Galectin Therapeutics), Galectin-3C (Mandal Med), Anginex, or OTX-008 (OncoEthix, Merck). Galectins are a family of proteins that bind to beta galactosidase sugars.

The Galectin family of proteins comprises at least of Galectin-1, Galectin-2, Galectin-3, Galectin-4, Galectin-7, and Galectin-8. Galectins are also referred to as S-type lectins, and are soluble proteins with, e.g., intracellular and extracellular functions.

Galectin-1 and Galectin-3 are highly expressed in various tumor types. Galectin-1 and Galectin-3 can promote angiogenesis and/or reprogram myeloid cells toward a pro-tumor phenotype, e.g., enhance immunosuppression from myeloid cells. Soluble Galectin-3 can also bind to and/or inactivate infiltrating T cells.

### Exemplary Galectin Inhibitors

In some embodiments, a Galectin inhibitor is an antibody molecule. In an embodiment, an antibody molecule is a monospecific antibody molecule and binds a single epitope. E.g., a monospecific antibody molecule having a plurality of immunoglobulin variable domain sequences, each of which binds the same epitope. In an embodiment, the Galectin inhibitor is an anti-Galectin, e.g., anti-Galectin-1 or anti-Galectin-3, antibody molecule. In some embodiments, the Galectin inhibitor is an anti-Galectin-1 antibody molecule. In some embodiments, the Galectin inhibitor is an anti-Galectin-3 antibody molecule.

In an embodiment an antibody molecule is a multispecific antibody molecule, e.g., it comprises a plurality of immunoglobulin variable domains sequences, wherein a first immunoglobulin variable domain sequence of the plurality has binding specificity for a first epitope and a second immunoglobulin variable domain sequence of the plurality has binding specificity for a second epitope. In an embodiment, the first and second epitopes are on the same antigen, e.g., the same protein (or subunit of a multimeric protein). In an embodiment, the first and second epitopes overlap. In an embodiment, the first and second epitopes do not overlap. In an embodiment, the first and second epitopes are on different antigens, e.g., the different proteins (or different subunits of a multimeric protein). In an embodiment, a multispecific antibody molecule comprises a third, fourth or fifth immunoglobulin variable domain. In an embodiment, a multispecific antibody molecule is a bispecific antibody molecule, a trispecific antibody molecule, or tetraspecific antibody molecule.

In an embodiment, the Galectin inhibitor is a multispecific antibody molecule. In an embodiment, a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In an embodiment, the first and second epitopes are on the same antigen, e.g., the same protein (or subunit of a multimeric protein). In an embodiment, the first and second epitopes overlap. In an embodiment, the first and second epitopes do not overlap. In an embodiment, the first and second epitopes are on different antigens, e.g., the different proteins (or different subunits of a multimeric protein). In an embodiment a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In an embodiment, a bispecific antibody molecule comprises a half antibody having binding specificity for a first epitope and a half antibody having binding specificity for a second epitope. In an embodiment, a bispecific antibody molecule comprises a half antibody, or fragment thereof, having binding specificity for a first epitope and a half antibody, or fragment thereof, having binding specificity for a second epitope. In an embodiment, a bispecific antibody molecule comprises a scFv, or fragment thereof, have binding specificity for a first epitope and a scFv, or fragment thereof, have binding specificity for a second epitope. In an embodiment, the Galectin inhibitor is a bispecific antibody molecule. In an embodiment, the first epitope is located on Galectin-1, and the second epitope is located on Galectin-3.

Protocols for generating bispecific or heterodimeric antibody molecules are known in the art; including but not limited to, for example, the “knob in a hole” approach described in, e.g., U.S. Pat. No. 5,731,168; the electrostatic steering Fc pairing as described in, e.g., WO 09/089004, WO 06/106905 and WO 2010/129304; Strand Exchange Engineered Domains (SEED) heterodimer formation as described in, e.g., WO 07/110205; Fab arm exchange as described in, e.g., WO 08/119353, WO 2011/131746, and WO 2013/060867; double antibody conjugate, e.g., by antibody cross-linking to generate a bi-specific structure using a heterobifunctional reagent having an amine-reactive group and a sulfhydryl reactive group as described in, e.g., U.S. Pat. No. 4,433,059; bispecific antibody determinants generated by recombining half antibodies (heavy-light chain pairs or Fabs) from different antibodies through cycle of reduction and oxidation of disulfide bonds between the two heavy chains, as described in, e.g., U.S. Pat. No. 4,444,878; trifunctional antibodies, e.g., three Fab′ fragments cross-linked through sulfhydryl reactive groups, as described in, e.g., U.S. Pat. No. 5,273,743; biosynthetic binding proteins, e.g., pair of scFvs cross-linked through C-terminal tails preferably through disulfide or amine-reactive chemical cross-linking, as described in, e.g., U.S. Pat. No. 5,534,254; bifunctional antibodies, e.g., Fab fragments with different binding specificities dimerized through leucine zippers (e.g., c-fos and c-jun) that have replaced the constant domain, as described in, e.g., U.S. Pat. No. 5,582,996; bispecific and oligospecific mono- and oligovalent receptors, e.g., VH—CH1 regions of two antibodies (two Fab fragments) linked through a polypeptide spacer between the CH1 region of one antibody and the VH region of the other antibody typically with associated light chains, as described in, e.g., U.S. Pat. No. 5,591,828; bispecific DNA-antibody conjugates, e.g., crosslinking of antibodies or Fab fragments through a double stranded piece of DNA, as described in, e.g., U.S. Pat. No. 5,635,602; bispecific fusion proteins, e.g., an expression construct containing two scFvs with a hydrophilic helical peptide linker between them and a full constant region, as described in, e.g., U.S. Pat. No. 5,637,481; multivalent and multispecific binding proteins, e.g., dimer of polypeptides having first domain with binding region of Ig heavy chain variable region, and second domain with binding region of Ig light chain variable region, generally termed diabodies (higher order structures are also disclosed creating bispecific, trispecific, or tetraspecific molecules, as described in, e.g., U.S. Pat. No. 5,837,242; minibody constructs with linked VL and VH chains further connected with peptide spacers to an antibody hinge region and CH3 region, which can be dimerized to form bispecific/multivalent molecules, as described in, e.g., U.S. Pat. No. 5,837,821; VH and VL domains linked with a short peptide linker (e.g., 5 or 10 amino acids) or no linker at all in either orientation, which can form dimers to form bispecific diabodies; trimers and tetramers, as described in, e.g., U.S. Pat. No. 5,844,094; String of VH domains (or VL domains in family members) connected by peptide linkages with crosslinkable groups at the C-terminus further associated with VL domains to form a series of FVs (or scFvs), as described in, e.g., U.S. Pat. No. 5,864,019; and single chain binding polypeptides with both a VH and a VL domain linked through a peptide linker are combined into multivalent structures through non-covalent or chemical crosslinking to form, e.g., homobivalent, heterobivalent, trivalent, and tetravalent structures using both scFV or diabody type format, as described in, e.g., U.S. Pat. No. 5,869,620. Additional exemplary multispecific and bispecific molecules and methods of making the same are found, for example, in U.S. Pat. Nos. 5,910,573, 5,932,448, 5,959,083, 5,989,830, 6,005,079, 6,239,259, 6,294,353, 6,333,396, 6,476,198, 6,511,663, 6,670,453, 6,743,896, 6,809,185, 6,833,441, 7,129,330, 7,183,076, 7,521,056, 7,527,787, 7,534,866, 7,612,181, US2002/004587A1, US2002/076406A1, US2002/103345A1, US2003/207346A1, US2003/211078A1, US2004/219643A1, US2004/220388A1, US2004/242847A1, US2005/003403A1, US2005/004352A1, US2005/069552A1, US2005/079170A1, US2005/100543A1, US2005/136049A1, US2005/136051A1, US2005/163782A1, US2005/266425A1, US2006/083747A1, US2006/120960A1, US2006/204493A1, US2006/263367A1, US2007/004909A1, US2007/087381A1, US2007/128150A1, US2007/141049A1, US2007/154901A1, US2007/274985A1, US2008/050370A1, US2008/069820A1, US2008/152645A1, US2008/171855A1, US2008/241884A1, US2008/254512A1, US2008/260738A1, US2009/130106A1, US2009/148905A1, US2009/155275A1, US2009/162359A1, US2009/162360A1, US2009/175851A1, US2009/175867A1, US2009/232811A1, US2009/234105A1, US2009/263392A1, US2009/274649A1, EP346087A2, WO00/06605A2, WO02/072635A2, WO04/081051A1, WO06/020258A2, WO2007/044887A2, WO2007/095338A2, WO2007/137760A2, WO2008/119353A1, WO2009/021754A2, WO2009/068630A1, WO91/03493A1, WO93/23537A1, WO94/09131A1, WO94/12625A2, WO95/09917A1, WO96/37621A2, WO99/64460A1. The contents of the above-referenced applications are incorporated herein by reference in their entireties.

In other embodiments, the anti-Galectin, e.g., anti-Galectin-1 or anti-Galectin-3, antibody molecule (e.g., a monospecific, bispecific, or multispecific antibody molecule) is covalently linked, e.g., fused, to another partner e.g., a protein, e.g., as a fusion molecule for example a fusion protein. In one embodiment, a bispecific antibody molecule has a first binding specificity to a first target (e.g., to Galectin-1), a second binding specificity to a second target (e.g., Galectin-3).

This invention provides an isolated nucleic acid molecule encoding the above antibody molecule, vectors and host cells thereof. The nucleic acid molecule includes but is not limited to RNA, genomic DNA and cDNA.

In some embodiments, a Galectin inhibitor is a peptide, e.g., protein, which can bind to, and inhibit Galectin, e.g., Galectin-1 or Galectin-3, function. In some embodiments, the Galectin inhibitor is a peptide which can bind to, and inhibit Galectin-3 function. In some embodiments, the Galectin inhibitor is the peptide Galectin-3C. In some embodiments, the Galectin inhibitor is a Galectin-3 inhibitor disclosed in U.S. Pat. No. 6,770,622, which is hereby incorporated by reference in its entirety.

Galectin-3C is an N-terminal truncated protein of Galectin-3, and functions, e.g., as a competitive inhibitor of Galectin-3. Galectin-3C prevents binding of endogenous Galectin-3 to e.g., laminin on the surface of, e.g., cancer cells, and other beta-galactosidase glycoconjugates in the extracellular matrix (ECM). Galectin-3C and other exemplary Galectin inhibiting peptides are disclosed in U.S. Pat. No. 6,770,622.

In some embodiments, Galectin-3C comprises the amino acid sequence of SEQ ID NO: 279, or an amino acid substantially identical (e.g., 90, 95 or 99%) identical thereto.

(SEQ ID NO: 279)
GAPAGPLIVPYNLPLPGGVVPRMLITILGTVKPNA
NRIALDFQRGNDVAFHFNPRFNENNRRVIVCNTKL
DNNWGREERQSVFPFESGKPFKIQVLVEPDHFKVA
VNDAHLLQYNHRVKKLNEISKLGISGDIDITSASY
TMI.

In some embodiments, the Galectin inhibitor is a peptide, which can bind to, and inhibit Galectin-1 function. In some embodiments, the Galectin inhibitor is the peptide Anginex: Anginex is an anti-angiongenic peptide that binds Galectin-1 (Salomonsson E, et al., (2011) Journal of Biological Chemistry, 286(16):13801-13804). Binding of Anginex to Galectin-1 can interfere with, e.g., the pro-angiongenic effects of Galectin-1.

In some embodiments, the Galectin, e.g., Galectin-1 or Galectin-3, inhibitor is a non-peptidic topomimetic molecule. In some embodiments, the non-peptidic topomimetic Galectin inhibitor is OTX-008 (OncoEthix). In some embodiments, the non-peptidic topomimetic is a non-peptidic topomimetic disclosed in U.S. Pat. No. 8,207,228, which is herein incorporated by reference in its entirety. OTX-008, also known as PTX-008 or Calixarene 0118, is a selective allosteric inhibitor of Galectin-1. OTX-008 has the chemical name: N-[2-(dimethylamino)ethyl]-2-{[26,27,28-tris({[2-(dimethylamino)ethyl]carbamoyl}methoxy)pentacyclo[19.3.1.1,7.1,0.15,]octacosa-1(25),3(28),4,6,9(27),1012,15,17,19(26),21,23-dodecaen-25-yl]oxy}acetamide.

In some embodiments, the Galectin, e.g., Galectin-1 or Galectin-3, inhibitor is a carbohydrate based compound. In some embodiments, the Galectin inhibitor is GR-MD-02 (Galectin Therapeutics).

In some embodiments, GR-MD-02 is a Galectin-3 inhibitor. GR-MD-02 is a galactose-pronged polysaccharide also referred to as, e.g., a galactoarabino-rhamnogalaturonate. GR-MD-02 and other galactose-pronged polymers, e.g., galactoarabino-rhamnogalaturonates, are disclosed in U.S. Pat. No. 8,236,780 and U.S. Publication 2014/0086932, the entire contents of which are herein incorporated by reference in their entirety.

### MEK Inhibitors

In some embodiments, a MEK inhibitor is used in combination with the compounds of Formula (I) or compounds of Embodiment 16, 17, or 35, or a pharmaceutically acceptable salt, hydrate, solvate, prodrug, stereoisomer, or tautomer thereof, for treating a disease, e.g., cancer. In some embodiments, the MEK inhibitor is chosen from Trametinib, selumetinib, AS703026, BIX 02189, BIX 02188, CI-1040, PD0325901, PD98059, U0126, XL-518, G-38963, or G02443714. In some embodiments, the MEK inhibitor is Trametinib.

### Exemplary MEK Inhibitors

In some embodiments, the MEK inhibitor is trametinib. Trametinib is also known as JTP-74057, TMT212, N-(3-{3-cyclopropyl-5-[(2-fluoro-4-iodophenyl)amino]-6,8-dimethyl-2,4,7-trioxo-3,4,6,7-tetrahydropyrido[4,3-d]pyrimidin-1(2H)-yl}phenyl)acetamide, or Mekinist (CAS Number 871700-17-3).

### Other Exemplary MEK Inhibitors

In some embodiments the MEK inhibitor comprises selumetinib which has the chemical name: (5-[(4-bromo-2-chlorophenyl)amino]-4-fluoro-N-(2-hydroxyethoxy)-1-methyl-TH-benzimidazole-6-carboxamide. Selumetinib is also known as AZD6244 or ARRY 142886, e.g., as described in PCT Publication No. WO2003077914.

In some embodiments, the MEK inhibitor comprises AS703026, BIX 02189 or BIX 02188.

In some embodiments, the MEK inhibitor comprises 2-[(2-Chloro-4-iodophenyl)amino]-N-(cyclopropylmethoxy)-3,4-difluoro-benzamide (also known as CI-1040 or PD184352), e.g., as described in PCT Publication No. WO2000035436).

In some embodiments, the MEK inhibitor comprises N-[(2R)-2,3-Dihydroxypropoxy]-3,4-difluoro-2-[(2-fluoro-4-iodophenyl)amino]-benzamide (also known as PD0325901), e.g., as described in PCT Publication No. WO2002006213).

In some embodiments, the MEK inhibitor comprises 2′-amino-3′-methoxyflavone (also known as PD98059) which is available from Biaffin GmbH & Co., KG, Germany.

In some embodiments, the MEK inhibitor comprises 2,3-bis[amino[(2-aminophenyl)thio]methylene]-butanedinitrile (also known as U0126), e.g., as described in U.S. Pat. No. 2,779,780).

In some embodiments, the MEK inhibitor comprises XL-518 (also known as GDC-0973) which has a CAS No. 1029872-29-4 and is available from ACC Corp.

In some embodiments, the MEK inhibitor comprises G-38963.

In some embodiments, the MEK inhibitor comprises G02443714 (also known as AS703206)

Additional examples of MEK inhibitors are disclosed in WO 2013/019906, WO 03/077914, WO 2005/121142, WO 2007/04415, WO 2008/024725 and WO 2009/085983, the contents of which are incorporated herein by reference. Further examples of MEK inhibitors include, but are not limited to, 2,3-Bis[amino[(2-aminophenyl)thio]methylene]-butanedinitrile (also known as U0126 and described in U.S. Pat. No. 2,779,780); (3S,4R,5Z,8S,9S,11E)-14-(Ethylamino)-8,9,16-trihydroxy-3,4-dimethyl-3,4,9, 19-tetrahydro-1H-2-benzoxacyclotetradecine-1,7(8H)-dione] (also known as E6201, described in PCT Publication No. WO2003076424); vemurafenib (PLX-4032, CAS 918504-65-1); (R)-3-(2,3-Dihydroxypropyl)-6-fluoro-5-(2-fluoro-4-iodophenylamino)-8-methylpyrido[2,3-d]pyrimidine-4,7(3H,8H)-dione (TAK-733, CAS 1035555-63-5); pimasertib (AS-703026, CAS 1204531-26-9); 2-(2-Fluoro-4-iodophenylamino)-N-(2-hydroxyethoxy)-1,5-dimethyl-6-oxo-1,6-dihydropyridine-3-carboxamide (AZD 8330); and 3,4-Difluoro-2-[(2-fluoro-4-iodophenyl)amino]-N-(2-hydroxyethoxy)-5-[(3-oxo-[1,2]oxazinan-2-yl)methyl]benzamide (CH 4987655 or Ro 4987655).

c-MET Inhibitors

In some embodiments, a c-MET inhibitor is used in combination with the compounds of Formula (I) or compounds of Embodiment 16, 17, or 35, or a pharmaceutically acceptable salt, hydrate, solvate, prodrug, stereoisomer, or tautomer thereof, for treating a disease, e.g., cancer. c-MET, a receptor tyrosine kinase overexpressed or mutated in many tumor cell types, plays key roles in tumor cell proliferation, survival, invasion, metastasis, and tumor angiogenesis. Inhibition of c-MET may induce cell death in tumor cells overexpressing c-MET protein or expressing constitutively activated c-MET protein.

In some embodiments, the c-MET inhibitor is chosen from capmatinib (INC280), JNJ-3887605, AMG 337, LY2801653, MSC2156119J, crizotinib, tivantinib, or golvatinib.

Exemplary c-MET Inhibitors

In some embodiments, the c-MET inhibitor comprises capmatinib (INC280), or a compound described in U.S. Pat. Nos. 7,767,675, and 8,461,330, which are incorporated by reference in their entirety.

Other Exemplary c-MET Inhibitors

In some embodiments, the c-MET inhibitor comprises JNJ-38877605. JNJ-38877605 is an orally available, small molecule inhibitor of c-Met. JNJ-38877605 selectively binds to c-MET, thereby inhibiting c-MET phosphorylation and disrupting c-Met signal transduction pathways. In some embodiments, the c-Met inhibitor is AMG 208. AMG 208 is a selective small-molecule inhibitor of c-MET. AMG 208 inhibits the ligand-dependent and ligand-independent activation of c-MET, inhibiting its tyrosine kinase activity, which may result in cell growth inhibition in tumors that overexpress c-Met.

In some embodiments, the c-Met inhibitor comprises AMG 337. AMG 337 is an orally bioavailable inhibitor of c-Met. AMG 337 selectively binds to c-MET, thereby disrupting c-MET signal transduction pathways.

In some embodiments, the c-Met inhibitor comprises LY2801653. LY2801653 is an orally available, small molecule inhibitor of c-Met. LY2801653 selectively binds to c-MET, thereby inhibiting c-MET phosphorylation and disrupting c-Met signal transduction pathways.

In some embodiments, c-Met inhibitor comprises MSC2156119J. MSC2156119J is an orally bioavailable inhibitor of c-Met. MSC2156119J selectively binds to c-MET, which inhibits c-MET phosphorylation and disrupts c-Met-mediated signal transduction pathways.

In some embodiments, the c-MET inhibitor is capmatinib. Capmatinib is also known as INCB028060. Capmatinib is an orally bioavailable inhibitor of c-MET. Capmatinib selectively binds to c-Met, thereby inhibiting c-Met phosphorylation and disrupting c-Met signal transduction pathways.

In some embodiments, the c-MET inhibitor comprises crizotinib. Crizotinib is also known as PF-02341066. Crizotinib is an orally available aminopyridine-based inhibitor of the receptor tyrosine kinase anaplastic lymphoma kinase (ALK) and the c-Met/hepatocyte growth factor receptor (HGFR). Crizotinib, in an ATP-competitive manner, binds to and inhibits ALK kinase and ALK fusion proteins. In addition, crizotinib inhibits c-Met kinase, and disrupts the c-Met signaling pathway. Altogether, this agent inhibits tumor cell growth.

In some embodiments, the c-MET inhibitor comprises golvatinib. Golvatinib is an orally bioavailable dual kinase inhibitor of c-MET and VEGFR-2 with potential antineoplastic activity. Golvatinib binds to and inhibits the activities of both c-MET and VEGFR-2, which may inhibit tumor cell growth and survival of tumor cells that overexpress these receptor tyrosine kinases.

In some embodiments, the c-MET inhibitor is tivantinib. Tivantinib is also known as ARQ 197. Tivantinib is an orally bioavailable small molecule inhibitor of c-MET. Tivantinib binds to the c-MET protein and disrupts c-Met signal transduction pathways, which may induce cell death in tumor cells overexpressing c-MET protein or expressing constitutively activated c-Met protein.

### TGF-β Inhibitors

In some embodiments, a transforming growth factor beta (also known as TGF-β TGFβ, TGFb, or TGF-beta, used interchangeably herein) inhibitor is used in combination with the compounds of Formula (I) or compounds of Embodiment 16, 17, or 35, or a pharmaceutically acceptable salt, hydrate, solvate, prodrug, stereoisomer, or tautomer thereof, for treating a disease, e.g., cancer. In certain embodiments, a combination described herein comprises a transforming growth factor beta (also known as TGF-β TGFβ, TGFb, or TGF-beta, used interchangeably herein) inhibitor.

TGF-β belongs to a large family of structurally-related cytokines including, e.g., bone morphogenetic proteins (BMPs), growth and differentiation factors, activins and inhibins. In some embodiments, the TGF-β inhibitors described herein can bind and/or inhibit one or more isoforms of TGF-β (e.g., one, two, or all of TGF-β1, TGF-β2, or TGF-β3).

Under normal conditions, TGF-β maintains homeostasis and limits the growth of epithelial, endothelial, neuronal and hematopoietic cell lineages, e.g., through the induction of anti-proliferative and apoptotic responses. Canonical and non-canonical signaling pathways are involved in cellular responses to TGF-β. Activation of the TGF-β/Smad canonical pathway can mediate the anti-proliferative effects of TGF-β. The non-canonical TGF-β pathway can activate additional intra-cellular pathways, e.g., mitogen-activated protein kinases (MAPK), phosphatidylinositol 3 kinase/Protein Kinase B, Rho-like GTPases (Tian et al. Cell Signal. 2011; 23(6):951-62; Blobe et al. N Engl J Med. 2000; 342(18):1350-8), thus modulating epithelial to mesenchymal transition (EMT) and/or cell motility.

Alterations of TGF-β signaling pathway are associated with human diseases, e.g., cancers, cardio-vascular diseases, fibrosis, reproductive disorders, and wound healing. Without wishing to be bound by theory, it is believed that in some embodiments, the role of TGF-β in cancer is dependent on the disease setting (e.g., tumor stage and genetic alteration) and/or cellular context. For example, in late stages of cancer, TGF-β can modulate a cancer-related process, e.g., by promoting tumor growth (e.g., inducing EMT), blocking anti-tumor immune responses, increasing tumor-associated fibrosis, or enhancing angiogenesis (Wakefield and Hill Nat Rev Cancer. 2013; 13(5):328-41). In certain embodiments, a combination comprising a TGF-0 inhibitor described herein is used to treat a cancer in a late stage, a metastatic cancer, or an advanced cancer.

Preclinical evidence indicates that TGF-0 plays an important role in immune regulation (Wojtowicz-Praga Invest New Drugs. 2003; 21(1):21-32; Yang et al. Trends Immunol. 2010; 31(6):220-7). TGF-0 can down-regulate the host-immune response via several mechanisms, e.g., shift of the T-helper balance toward Th2 immune phenotype; inhibition of anti-tumoral Th1 type response and M1-type macrophages; suppression of cytotoxic CD8+T lymphocytes (CTL), NK lymphocytes and dendritic cell functions, generation of CD4+CD25+T-regulatory cells; or promotion of M2-type macrophages with pro-tumoral activity mediated by secretion of immunosuppressive cytokines (e.g., IL10 or VEGF), pro-inflammatory cytokines (e.g., IL6, TNFα, or IL1) and generation of reactive oxygen species (ROS) with genotoxic activity (Yang et al. Trends Immunol. 2010; 31(6):220-7; Truty and Urrutia Pancreatology. 2007; 7(5-6):423-35; Achyut et al Gastroenterology. 2011; 141(4):1167-78).

### Exemplary TGF-β Inhibitors

In some embodiments, the TGF-β inhibitor comprises XOMA 089, or a compound disclosed in International Application Publication No. WO 2012/167143, which is incorporated by reference in its entirety.

XOMA 089 is also known as XPA.42.089. XOMA 089 is a fully human monoclonal antibody that specifically binds and neutralizes TGF-beta 1 and 2 ligands.

The heavy chain variable region of XOMA 089 has the amino acid sequence of: QVQLVQSGAEVKKPGSSVKVSCKASGGTFSSYAISWVRQAPGQGLEWMGGIIPIFGTAN YAQKFQGRVTITADESTSTAYMELSSLRSEDTAVYYCARGLWEVRALPSVYWGQGTLV TVSS (SEQ ID NO: 284) (disclosed as SEQ ID NO: 6 in WO 2012/167143). The light chain variable region of XOMA 089 has the amino acid sequence of: SYELTQPPSVSVAPGQTARITCGANDIGSKSVHWYQQKAGQAPVLVVSEDIIRPSGIPERI SGSNSGNTATLTISRVEAGDEADYYCQVWDRDSDQYVFGTGTKVTVLG (SEQ ID NO: 285) (disclosed as SEQ ID NO: 8 in WO 2012/167143).

XOMA 089 binds with high affinity to the human TGF-β isoforms. Generally, XOMA 089 binds with high affinity to TGF-β1 and TGF-β2, and to a lesser extent to TGF-β3. In Biacore assays, the K_{D} of XOMA 089 on human TGF-β is 14.6 μM for TGF-β1, 67.3 μM for TGF-β2, and 948 μM for TGF-β3. Given the high affinity binding to all three TGF-β isoforms, in certain embodiments, XOMA 089 is expected to bind to TGF-β1, 2 and 3 at a dose of XOMA 089 as described herein. XOMA 089 cross-reacts with rodent and cynomolgus monkey TGF-β and shows functional activity in vitro and in vivo, making rodent and cynomolgus monkey relevant species for toxicology studies.

### Other Exemplary TGF-β Inhibitors

In some embodiments, the TGF-0 inhibitor comprises fresolimumab (CAS Registry Number: 948564-73-6). Fresolimumab is also known as GC1008. Fresolimumab is a human monoclonal antibody that binds to and inhibits TGF-beta isoforms 1, 2 and 3.

The heavy chain of fresolimumab has the amino acid sequence of:

(SEQ ID NO: 280)
QVQLVQSGAEVKKPGSSVKVSCKASGYTFSSNVIS
WVRQAPGQGLEWMGGVIPIVDIANYAQRFKGRVTI
TADESTSTTYMELSSLRSEDTAVYYCASTLGLVLD
AMDYWGQGTLVTVSSASTKGPSVFPLAPCSRSTSE
STAALGCLVKDYFPEPVTVSWNSGALTSGVHTFPA
VLQSSGLYSLSSVVTVPSSSLGTKTYTCNVDHKPS
NTKVDKRVESKYGPPCPSCPAPEFLGGPSVFLFPP
KPKDTLMISRTPEVTCVVVDVSQEDPEVQFNWYVD
GVEVHNAKTKPREEQFNSTYRVVSVLTVLHQDWLN
GKEYKCKVSNKGLPSSIEKTISKAKGQPREPQVYT
LPPSQEEMTKNQVSLTCLVKGFYPSDIAVEWESNG
QPENNYKTTPPVLDSDGSFFLYSRLTVDKSRWQEG
NVFSCSVMHEALHNHYTQKSLSLSLGK.

The light chain of fresolimumab has the amino acid sequence of:

(SEQ ID NO: 281)
ETVLTQSPGTLSLSPGERATLSCRASQSLGSSYLA
WYQQKPGQAPRLLIYGASSRAPGIPDRFSGSGSGT
DFTLTISRLEPEDFAVYYCQQYADSPITFGQGTRL
EIKRTVAAPSVFIFPPSDEQLKSGTASVVCLLNNF
YPREAKVQWKVDNALQSGNSQESVTEQDSKDSTYS
LSSTLTLSKADYEKHKVYACEVTHQGLSSPVTKSF
NRGEC.

Fresolimumab is disclosed, e.g., in International Application Publication No. WO 2006/086469, and U.S. Pat. Nos. 8,383,780 and 8,591,901, which are incorporated by reference in their entirety.

### IL-1β Inhibitors

The Interleukin-1 (IL-1) family of cytokines is a group of secreted pleotropic cytokines with a central role in inflammation and immune response. Increases in IL-1 are observed in multiple clinical settings including cancer (Apte et al. (2006) Cancer Metastasis Rev. p. 387-408; Dinarello (2010) Eur. J. Immunol. p. 599-606). The IL-1 family comprises, inter alia, IL-1 beta (IL-1b), and IL-1alpha (IL-1a). IL-1b is elevated in lung, breast and colorectal cancer (Voronov et al. (2014) Front Physiol. p. 114) and is associated with poor prognosis (Apte et al. (2000) Adv. Exp. Med. Biol. p. 277-88). Without wishing to be bound by theory, it is believed that in some embodiments, secreted IL-1b, derived from the tumor microenvironment and by malignant cells, promotes tumor cell proliferation, increases invasiveness and dampens anti-tumor immune response, in part by recruiting inhibitory neutrophils (Apte et al. (2006) Cancer Metastasis Rev. p. 387-408; Miller et al. (2007) J. Immunol. p. 6933-42). Experimental data indicate that inhibition of IL-1b results in a decrease in tumor burden and metastasis (Voronov et al. (2003) Proc. Natl. Acad. Sci. U.S.A. p. 2645-50).

In some embodiments, an interleukin-1 beta (IL-1β) inhibitor is used in combination with the compounds of Formula (I) or compounds of Embodiment 16, 17, or 35, or a pharmaceutically acceptable salt, hydrate, solvate, prodrug, stereoisomer, or tautomer thereof, for treating a disease, e.g., cancer. In some embodiments, the IL-1β inhibitor is chosen from canakinumab, gevokizumab, Anakinra, or Rilonacept. In some embodiments, the IL-1β inhibitor is canakinumab.

### Exemplary IL-1β Inhibitors

In some embodiments, the IL-1β inhibitor is canakinumab. Canakinumab is also known as ACZ885 or ILARIS®. Canakinumab is a human monoclonal IgG1/κ antibody that neutralizes the bioactivity of human IL-1β.

Canakinumab is disclosed, e.g., in WO 2002/16436, U.S. Pat. No. 7,446,175, and EP 1313769. The heavy chain variable region of canakinumab has the amino acid sequence of: MEFGLSWVFLVALLRGVQCQVQLVESGGGVVQPGRSLRLSCAASGFTFSVYGMNWVR QAPGKGLEWVAIIWYDGDNQYYADSVKGRFTISRDNSKNTLYLQMNGLRAEDTAVYY CARDLRTGPFDYWGQGTLVTVSS (SEQ ID NO: 282) (disclosed as SEQ ID NO: 1 in U.S. Pat. No. 7,446,175). The light chain variable region of canakinumab has the amino acid sequence of: MLPSQLIGFLLLWVPASRGEIVLTQSPDFQSVTPKEKVTITCRASQSIGSSLHWYQQKPD QSPKLLIKYASQSFSGVPSRFSGSGSGTDFTLTINSLEAEDAAAYYCHQSSSLPFTFGPGT KVDIK (SEQ ID NO: 283) (disclosed as SEQ ID NO: 2 in U.S. Pat. No. 7,446,175).

Canakinumab has been used, e.g., for the treatment of Cryopyrin Associated Periodic Syndromes (CAPS), in adults and children, for the treatment of systemic juvenile idiopathic arthritis (SJIA), for the symptomatic treatment of acute gouty arthritis attacks in adults, and for other IL-1β driven inflammatory diseases. Without wishing to be bound by theory, it is believed that in some embodiments, IL-10 inhibitors, e.g., canakinumab, can increase anti-tumor immune response, e.g., by blocking one or more functions of IL-1b including, e.g., recruitment of immunosuppressive neutrophils to the tumor microenvironment, stimulation of tumor angiogenesis, and/or promotion of metastasis (Dinarello (2010) Eur. J. Immunol. p. 599-606).

In some embodiments, the combination described herein includes an IL-1β inhibitor, canakinumab, or a compound disclosed in WO 2002/16436, and an inhibitor of an immune checkpoint molecule, e.g., an inhibitor of PD-1 (e.g., an anti-PD-1 antibody molecule). IL-1 is a secreted pleotropic cytokine with a central role in inflammation and immune response. Increases in IL-1 are observed in multiple clinical settings including cancer (Apte et al. (2006) Cancer Metastasis Rev. p. 387-408; Dinarello (2010) Eur. J. Immunol. p. 599-606). IL-1b is elevated in lung, breast and colorectal cancer (Voronov et al. (2014) Front Physiol. p. 114) and is associated with poor prognosis (Apte et al. (2000) Adv. Exp. Med. Biol. p. 277-88). Without wishing to be bound by theory, it is believed that in some embodiments, secreted IL-1b, derived from the tumor microenvironment and by malignant cells, promotes tumor cell proliferation, increases invasiveness and dampens anti-tumor immune response, in part by recruiting inhibitory neutrophils (Apte et al. (2006) Cancer Metastasis Rev. p. 387-408; Miller et al. (2007) J. Immunol. p. 6933-42). Experimental data indicate that inhibition of IL-1b results in a decrease in tumor burden and metastasis (Voronov et al. (2003) Proc. Natl. Acad. Sci. U.S.A. p. 2645-50). Canakinumab can bind IL-1b and inhibit IL-1-mediated signaling. Accordingly, in certain embodiments, an IL-1β inhibitor, e.g., canakinumab, enhances, or is used to enhance, an immune-mediated anti-tumor effect of an inhibitor of PD-1 (e.g., an anti-PD-1 antibody molecule).

In some embodiments, the IL-1β inhibitor, canakinumab, or a compound disclosed in WO 2002/16436, and the inhibitor of an immune checkpoint molecule, e.g., an inhibitor of PD-1 (e.g., an anti-PD-1 antibody molecule), each is administered at a dose and/or on a time schedule, that in combination, achieves a desired anti-tumor activity.

### MDM2 Inhibitors

In some embodiments, a mouse double minute 2 homolog (MDM2) inhibitor is used in combination with the compounds of Formula (I) or compounds of Embodiment 16, 17, or 35, or a pharmaceutically acceptable salt, hydrate, solvate, prodrug, stereoisomer, or tautomer thereof, for treating a disease, e.g., cancer. The human homolog of MDM2 is also known as HDM2. In some embodiments, an MDM2 inhibitor described herein is also known as a HDM2 inhibitor. In some embodiments, the MDM2 inhibitor is chosen from HDM201 or CGM097.

In an embodiment the MDM2 inhibitor comprises (S)-1-(4-chlorophenyl)-7-isopropoxy-6-methoxy-2-(4-(methyl(((1r,4S)-4-(4-methyl-3-oxopiperazin-1-yl)cyclohexyl)methyl)amino)phenyl)-1,2-dihydroisoquinolin-3(4H)-one (also known as CGM097) or a compound disclosed in PCT Publication No. WO 2011/076786 to treat a disorder, e.g., a disorder described herein). In one embodiment, a therapeutic agent disclosed herein is used in combination with CGM097.

In an embodiment, an MDM2 inhibitor comprises an inhibitor of p53 and/or a p53/Mdm2 interaction. In an embodiment, the MDM2 inhibitor comprises (S)-5-(5-chloro-1-methyl-2-oxo-1,2-dihydropyridin-3-yl)-6-(4-chlorophenyl)-2-(2,4-dimethoxypyrimidin-5-yl)-1-isopropyl-5,6-dihydropyrrolo[3,4-d]imidazol-4(1H)-one (also known as HDM201), or a compound disclosed in PCT Publication No. WO2013/111105 to treat a disorder, e.g., a disorder described herein. In one embodiment, a therapeutic agent disclosed herein is used in combination with HDM201. In some embodiments, HDM201 is administered orally.

In one embodiment, the combination disclosed herein is suitable for the treatment of cancer in vivo. For example, the combination can be used to inhibit the growth of cancerous tumors. The combination can also be used in combination with one or more of: a standard of care treatment (e.g., for cancers or infectious disorders), a vaccine (e.g., a therapeutic cancer vaccine), a cell therapy, a radiation therapy, surgery, or any other therapeutic agent or modality, to treat a disorder herein. For example, to achieve antigen-specific enhancement of immunity, the combination can be administered together with an antigen of interest.

### EXAMPLES

The disclosure is further illustrated by the following examples and synthesis schemes, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

Compounds of the present disclosure may be prepared by methods known in the art of organic synthesis. In all of the methods it is understood that protecting groups for sensitive or reactive groups may be employed where necessary in accordance with general principles of chemistry. Protecting groups are manipulated according to standard methods of organic synthesis (T. W. Green and P. G. M. Wuts (1999) Protective Groups in Organic Synthesis, 3rd edition, John Wiley & Sons). These groups are removed at a convenient stage of the compound synthesis using methods that are readily apparent to those skilled in the art.

### Analytical Methods, Materials, and Instrumentation

Unless otherwise noted, reagents and solvents were used as received from commercial suppliers. Proton nuclear magnetic resonance (NMR) spectra were obtained on either Bruker Avance spectrometer or Varian Oxford 400 MHz spectrometer unless otherwise noted. Spectra are given in ppm (δ) and coupling constants, J, are reported in Hertz. Tetramethylsilane (TMS) was used as an internal standard. Chemical shifts are reported in ppm relative to dimethyl sulfoxide (δ 2.50), methanol (δ 3.31), chloroform (δ 7.26) or other solvent as indicated in NMR spectral data. A small amount of the dry sample (2-5 mg) is dissolved in an appropriate deuterated solvent (1 mL). The chemical names were generated using ChemBioDraw Ultra v12 or v17 from CambridgeSoft.

Mass spectra (ESI-MS) were collected using a Waters System (Acquity UPLC and a Micromass ZQ mass spectrometer) or Agilent-1260 Infinity (6120 Quadrupole); all masses reported are the m/z of the protonated parent ions unless recorded otherwise. The sample was dissolved in a suitable solvent such as MeCN, DMSO, or MeOH and was injected directly into the column using an automated sample handler. The analysis is performed on Waters Acquity UPLC system (Column: Waters Acquity UPLC BEH C18 1.7 μm, 2.1×30 mm; Flow rate: 1 mL/min; 55° C. (column temperature); Solvent A: 0.05% formic acid in water, Solvent B: 0.04% formic acid in MeOH; gradient 95% Solvent A from 0 to 0.10 min; 95% Solvent A to 20% Solvent A from 0.10 to 0.50 min; 20% Solvent A to 5% Solvent A from 0.50 to 0.60 min; hold at 5% Solvent A from 0.6 min to 0.8 min; 5% Solvent A to 95% Solvent A from 0.80 to 0.90 min; and hold 95% Solvent A from 0.90 to 1.15 min.

Abbreviations used in the following examples and elsewhere herein are:
AC₅₀ half maximal active concentration
aq. aqueous
br broad
Cs₂CO₃ cesium carbonate
cat. catalyst
d doublet
DAST Diethylaminosulfur trifluoride
di-tBu-bipy 4,4′-di-tert-butyl-2,2′-dipyridyl
dd doublet of doublets
ddd doublet of doublet of doublets
ddq doublet of doublet of quartets
ddt doublet of doublet of triplets
dq doublet of quartets
dt doublet of triplets
dtd doublet of triplet of doublets
DCM dichloromethane
DMA N,N-dimethylacetamide
DME 1,2-Dimethoxyethane
DMF N,N-dimethylformamide
DMSO dimethylsulfoxide
dppf 1,1′-Bis(diphenylphosphino)ferrocene
EC₅₀ half maximal effective concentration
Et₂O diethyl ether
Et₃N triethylamine
EtOAc ethyl acetate
HCl hydrogen chloride
hept heptet
HPLC high performance liquid chromatography
h or hr hour
HRMS high resolution mass spectrometry
g gram
IC₅₀ half maximal inhibitory concentration
K₂CO₃ potassium carbonate
KI potassium iodide
K₃PO₄ tripotassium phosphate
LCMS liquid chromatography mass spectrometry
LiHMDS Lithium bis(trimethylsilyl)amide
m multiplet
MeCN acetonitrile
MeOH methanol
mg milligram
MgCl₂ magnesium chloride
MHz megahertz
min minutes
mL milliliter
mmol millimole
M molar
MS mass spectrometry
NaBH(OAc)₃ sodium triacetoxyborohydride
NaHCO₃ sodium bicarbonate
Na₂SO₄ sodium sulfate
NiBr₂·DME nickel (II) bromide ethylene glycol dimethyl ether complex
NiI₂ nickel (II) iodide
NMR Nuclear magnetic resonance
Pd(dppf)Cl₂·DCM [1,1′-Bis(diphenylphosphino)ferrocene]dichloropalladium(II), complex with dichloromethane
PtO₂ platinum (IV) oxide
q quartet
qd quartet of doublets
quint quintet
quintd quintet of doublets
rt room temperature
Rt retention time
s singlet
t triplet
TEA triethylamine
td triplet of doublets
tdd triplet of doublet of doublets
THF tetrahydrofuran
Ts tosyl
tt triplet of triplets
ttd triplet of triplet of doublets
TLC thin-layer chromatography
UPLC ultra-Performance Liquid Chromatography
XPhos Pd G2 chloro(2-dicyclohexylphosphino-2′,4′,6′-triisopropyl-1,1′-biphenyl)[2-(2′-amino-1,1′-biphenyl)]palladium(II)

### Example 1: 3-(1-oxo-5-(piperidin-4-yl)isoindolin-2-yl)piperidine-2,6-dione (INT-G)

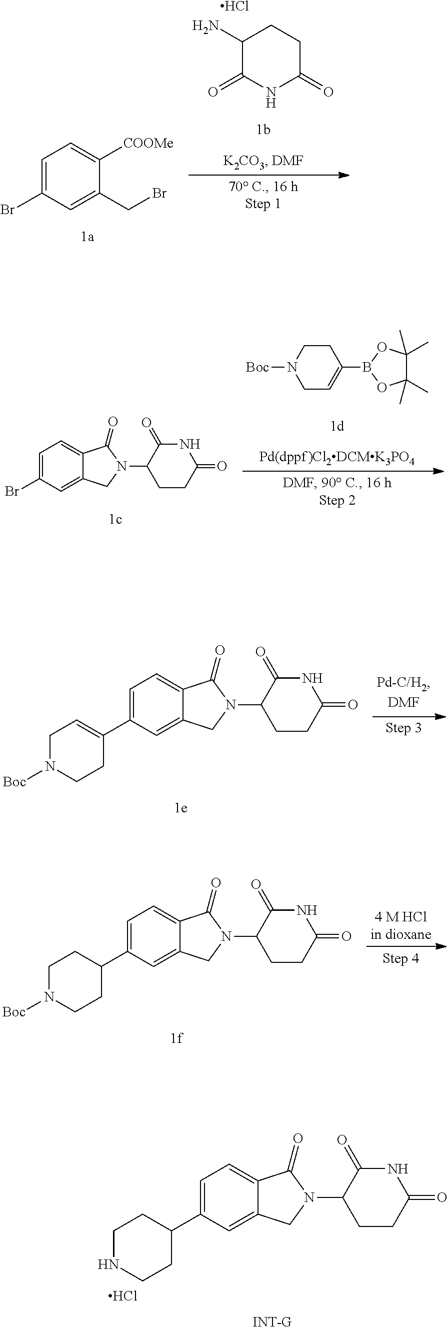

### Step 1. 3-(5-bromo-1-oxoisoindolin-2-yl)piperidine-2,6-dione (1c)

Intermediate 1a was prepared as reported in U.S. Patent Application US 2009/0142297.

To a stirred solution of methyl 4-bromo-2-(bromomethyl)benzoate (1a, 15 g, 48.7 mmol) in DMF (150 mL) was added 3-aminopiperidine-2,6-dioneHCl salt (1b, 6.9 g, 53.6 mmol) and K₂CO₃ (20.2 g, 146.1 mmol). The resulting mixture was stirred at 70° C. for 16 h after which time the reaction mixture was cooled to rt and then concentrated to dryness. To the resulting residue, water was added and the mixture stirred at rt for 30 min. The resultant solid was filtered and washed with ether and ethyl acetate. The solid was dried under vacuum filtration to afford 1c (10.6 g, 32.9 mmol, 67% yield). MS [M+H]⁺=323.0. ¹H NMR (400 MHz, DMSO-d₆) 10.99 (s, 1H), 7.91-7.88 (m, 1H), 7.72 (dd, J=8.1, 1.6 Hz, 1H), 7.67 (d, J=8.0 Hz, 1H), 5.11 (dd, J=13.3, 5.1 Hz, 1H), 4.47 (d, J=17.7 Hz, 1H), 4.34 (d, J=17.7 Hz, 1H), 2.98-2.83 (m, 1H), 2.65-2.55 (m, 1H), 2.45-2.29 (m, 1H), 2.01 (dtd, J=12.7, 5.3, 2.3 Hz, 1H).

### Step 2. tert-Butyl-4-(2-(2,6-dioxopiperidin-3-yl)-1-oxoisoindolin-5-yl)-3,6-dihydropyridine-1(2H)-carboxylate (1e)

A solution of 1c (1.80 g, 5.60 mmol) in DMF (10 mL) in a sealed tube was purged with argon for 5 min prior to addition of 3,6-dihydro-2H-pyridine-1-tert-butoxycarbonyl-4-boronic acid pinacol ester (1d, 2.2 g, 7.2 mmol), K₃PO₄ (1.42 g, 6.7 mmol) and Pd(dppf)Cl₂·DCM (227 mg, 0.28 mmol). The reaction mixture was again purged with argon for 5 min and then stirred at 90° C. for 16 h. After this time the reaction mixture was cooled to rt and then concentrated under reduced pressure. Water was added to the residue, which was then extracted with EtOAc. The organic layer was washed with brine, dried over Na₂SO₄, filtered, and then concentrated under a reduced pressure. The crude compound was purified by silica gel chromatography, eluting with 70-80% of EtOAc in hexanes, to afford 1e as a light brown solid (1.0 g, 2.4 mmol, 42% yield). MS [M+H]⁺=426.3.

### Step 3. tert-Butyl 4-(2-(2,6-dioxopiperidin-3-yl)-1-oxoisoindolin-5-yl)piperidine-1-carboxylate (1f)

To a stirred solution of 1e (1.0 g, 2.35 mmol) in DMF (20 mL) was added 10% Pd/C (150 mg) and the mixture was stirred under a hydrogen atmosphere (balloon) at rt for 6 h. The reaction mixture was then filtered through a bed of Celite® filter aid. The filtrate was concentrated under reduced pressure affording if as an off-white solid (0.85 g, 1.97 mmol, 84% yield). MS [M-tBu+H]⁺=372.3. ¹H NMR (400 MHz, CDCl₃) δ 8.40 (s, 1H), 7.81 (d, J=7.9 Hz, 1H), 7.32 (d, J 8.0 Hz, 1H), 7.29 (s, 1H), 5.22 (dd, J=13.3, 5.1 Hz, 1H), 4.46 (d, J=16.0 Hz, 1H), 4.31 (d, J 16.1 Hz, 1H), 4.27 (d, J=16.2 Hz, 2H), 2.97-2.67 (m, 5H), 2.41-2.26 (m, 1H), 2.23-2.13 (m, 1H), 1.83 (d, J=12.6 Hz, 2H), 1.71-1.55 (m, 2H), 1.48 (s, 9H).

### Step 4. 3-(1-oxo-5-(piperidin-4-yl)isoindolin-2-yl)piperidine-2,6-dione hydrochloride (INT-G)

To a stirred solution of if (0.85 g, 2.0 mmol) in dioxane (10 mL) was added 4M HCl in dioxane (5.0 mL). The reaction mixture was then stirred at rt for 2 h. The reaction mixture was concentrated under reduced pressure to afford the HCl salt of desired compound INT-G as an off-white solid (0.65 g, 1.8 mmol, 90% yield, hydrochloride salt). MS [M+H]=328.3. ¹H NMR (400 MHz, DMSO-d₆): 10.99 (s, 1H), 9.28 (s, 1H), 7.70 (d, J=7.8 Hz, 1H), 7.46 (s, 1H), 7.39 (d, J 8.0 Hz, 1H), 5.74 (s, 1H), 5.11 (dd, J=13.3, 5.2 Hz, 1H), 4.46 (d, J=17.3 Hz, 1H), 4.32 (d, J 17.3 Hz, 1H), 3.36 (d, J=11.5 Hz, 2H), 3.10-2.86 (m, 4H), 2.61 (d, J=14.8 Hz, 1H), 2.39 (qd, J 13.2, 4.3 Hz, 1H), 2.14-1.79 (m, 5H).

Conversion of 1c to 1f was also achieved in a single step via Negishi coupling using the following procedure:

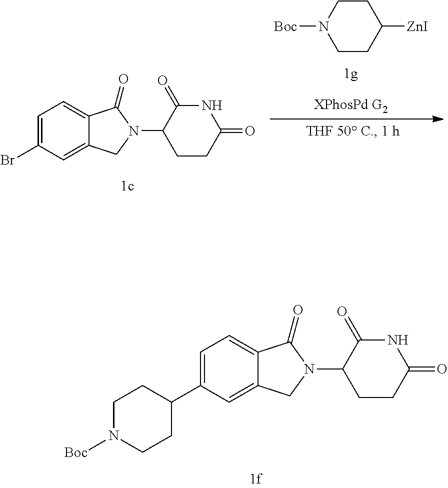

1-(tert-butoxycarbonyl)piperidin-4-yl)zinc(II) iodide (1g) was prepared as reported in Corley, E. G., et al., J Org. Chem. 2004, 69, 5120.

A mixture of 1c (41 mg, 0.125 mmol) and XPhos Pd cycle G2 (15 mg, 0.019 mmol) in THF (1.5 mL) was purged with nitrogen prior to addition of (1-(tert-butoxycarbonyl)piperidin-4-yl)zinc(II) iodide (1 g, 142 mg, 0.376 mmol) in THF (0.7 mL). The resulting mixture was stirred at 50° C. for 1 h after which time the reaction was cooled to rt, quenched with brine, and extracted with EtOAc. The organic layer was passed through a phase separator and concentrated. The crude material was purified by silica gel chromatography (eluting with 0-100% EtOAc in heptane) to afford if as a white solid (30 mg, 0.070 mmol, 56% yield).

Conversion of 1c to 1f was also achieved in a single step via an alternative reductive cross-coupling procedure:

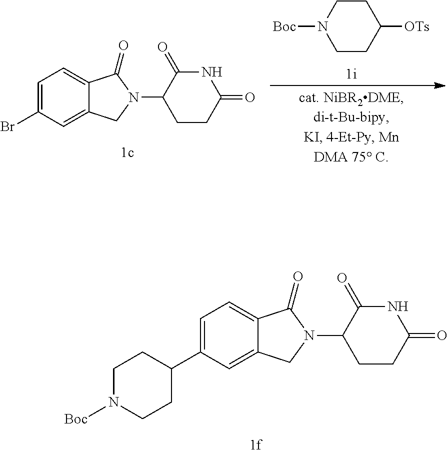 [SMILES: O=C1CCC(N2Cc3cc(Br)ccc3C2=O)C(=O)N1 ; Cc1ccc(S(=O)(=O)OC2CCN(C(=O)OC(C)(C)C)CC2)cc1 ; CC(C)(C)OC(=O)N1CCC(c2ccc3c(c2)CN(C2CCC(=O)NC2=O)C3=O)CC1]

To crude 1c (84% pure, 34 mg, 0.088 mmol), tert-butyl 4-(tosyloxy)piperidine-1-carboxylate (l1, 38 mg, 0.11 mmol), NiBr₂·DME (2.7 mg, 8.8 μmol), di-t-Bu-bipy (2.4 mg, 8.8 μmol), KI (15 mg, 0.09 mmol) and manganese powder (10 mg, 0.18 mmol) in DMA (0.50 mL) was added 4-ethylpyridine (10 μL, 0.088 mmol) and the reaction mixture was stirred vigorously at 75° C. for 5 h. The reaction mixture was filtered through a short pad of Celite® filter aid and eluted with MeCN. The obtained solution was concentrated by azeotroping with heptane. The crude product was purified via chromatography on silica gel eluting with MeOH in DCM to afford if (21.7 mg, 0.051 mmol, 57% yield) as a white solid.

In a similar fashion, intermediate 1f could be obtained from intermediate 29d, (the route for synthesis of 29d is outlined in Example 29):

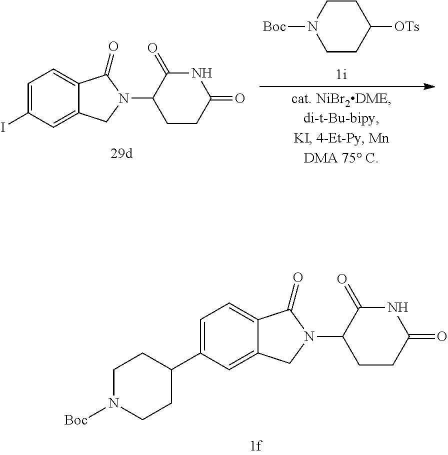 [SMILES: O=C1CCC(N2Cc3cc(I)ccc3C2=O)C(=O)N1 ; Cc1ccc(S(=O)(=O)OC2CCN(C(=O)OC(C)(C)C)CC2)cc1 ; CC(C)(C)OC(=O)N1CCC(c2ccc3c(c2)CN(C2CCC(=O)NC2=O)C3=O)CC1]

To 29d (48 mg, 0.13 mmol), tert-butyl 4-(tosyloxy)piperidine-1-carboxylate (1i, 55 mg, 0.16 mmol), NiBr₂·DME (4.0 mg, 0.013 mmol), di-t-Bu-bipy (3.5 mg, 0.013 mmol), KI (22 mg, 0.13 mmol) and manganese powder (14 mg, 0.26 mmol) in DMA (0.67 mL) was added 4-ethylpyridine (0.015 mL, 0.14 mmol) and the reaction mixture was stirred vigorously at 75° C. for 5 h. The reaction mixture was filtered through a short pad of Celite® filter aid and eluted with MeCN. The obtained solution was concentrated by azeotroping with heptane. The crude product was purified via chromatography on silica gel eluting with MeOH in DCM to afford if (33.3 mg, 0.078 mmol, 60% yield) as a white solid.

### Example 2: 3-(1-oxo-5-(1-(1-(tetrahydro-2H-pyran-4-yl)ethyl)piperidin-4-yl)isoindolin-2-yl)piperidine-2,6-dione (I-30)

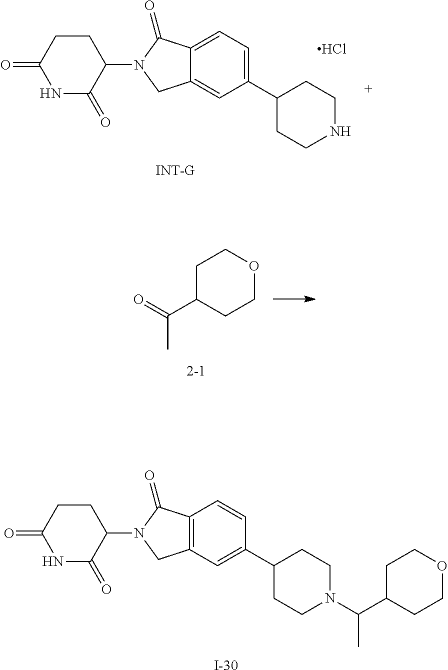

To a stirred solution of INT-G (200 mg, 0.55 mmol) and 1-(tetrahydro-2H-pyran-4-yl)ethan-1-one (2-1, 210 mg, 1.64 mmol) in DMSO (10 mL), was added titanium isopropoxide (310 mg, 1.09 mmol). The resulting mixture was stirred at rt for 16 h and then NaBH₃(CN) (69 mg, 2.0 mmol) was added and stirring was continued at rt for an additional 16 h. Upon complete consumption of the starting materials, the reaction mixture was quenched with aq. NaHCO₃ and extracted with EtOAc (2×50 mL). The combined organic extracts were washed with brine, dried over Na₂SO₄, filtered, and concentrated under reduced pressure. The obtained crude material was purified by silica gel column chromatography eluting with 7% MeOH in DCM to afford 1-30 as an off-white solid (110 mg, 0.25 mmol, 46% yield). MS [M+H]⁺=440.1. ¹H NMR (300 MHz, DMSO-d₆): δ 10.99 (s, 1H), 7.63 (d, J=7.2 Hz, 1H), 7.48 (s, 1H), 7.38 (d, J=7.8 Hz, 1H), 5.08 (dd, J=13.2, 4.8 Hz, 1H), 4.34 (d, J=17.2 Hz, 1H), 4.19 (d, J=17.2 Hz, 1H), 3.86-3.83 (m, 2H), 3.45-3.25 (m, 2H), 2.98-2.85 (m, 1H), 2.78-2.55 (m, 5H), 2.45-2.15 (m, 3H), 2.08-1.82 (m, 2H), 1.72-1.51 (m, 6H), 1.18-1.04 (m, 2H), 0.89 (d, J=6.9 Hz, 3H).

### Example 3: 3-(5-(1-(1-(1-ethyl-1H-pyrazol-4-yl)ethyl)piperidin-4-yl)-1-oxoisoindolin-2-yl)piperidine-2,6-dione (I-31)

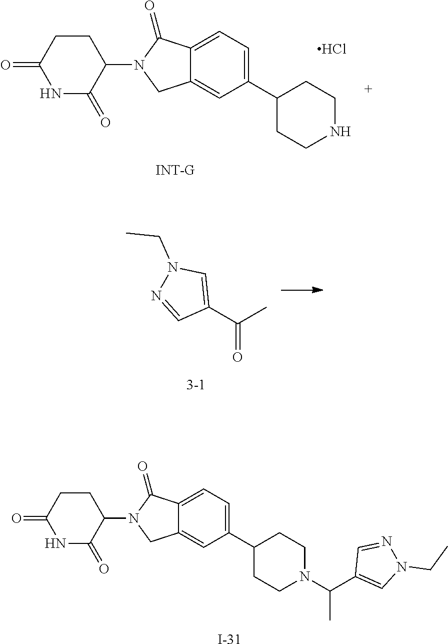

To a stirred solution of INT-G (400 mg, 0.09 mmol) and 3-1 (303 mg, 2.20 mmol) in DMSO (20 mL), was added titanium isopropoxide (1.24 g, 4.40 mmol). The resulting mixture was stirred at rt for 16 h and then NaBH₃(CN) (138 mg, 2.20 mmol) was added and stirring was continued at rt for an additional 16 h. Upon complete consumption of the starting materials, the reaction mixture was quenched with aq. NaHCO₃ and extracted with EtOAc (2×75 mL). The combined organic extracts were washed with brine, dried over Na₂SO₄, filtered, and concentrated under reduced pressure. The obtained crude material was purified by silica gel column chromatography eluting with 7% MeOH in DCM to afford I-31 as an off-white solid (170 mg, 0.38 mmol, 34% yield). MS [M+H]=450.5. ¹H NMR (400 MHz, DMSO-d₆): δ 10.99 (s, 1H), 7.63 (d, J=7.6 Hz, 1H), 7.58 (br s, 1H), 7.46 (s, 1H), 7.36 (d, J=8.0 HZ, 1H), 7.32 (br s, 1H), 5.09 (dd, J=13.2, 4.8 Hz, 1H), 4.42 (d, J=17.2 Hz, 1H), 4.27 (d, J=17.2 Hz, 1H), 4.09-4.06 (m, 2H), 3.70 (br s, 1H), 2.90-267 (m, 3H), 2.60-2.32 (m, 4H), 1.99-1.96 (m, 3H), 1.79-1.62 (m, 3H), 1.37-1.23 (m, 6H).

### Example 4: 3-(5-(1-(1-(1-ethyl-1H-pyrazol-4-yl)propyl)piperidin-4-yl)-1-oxoisoindolin-2-yl)piperidine-2,6-dione (I-32)

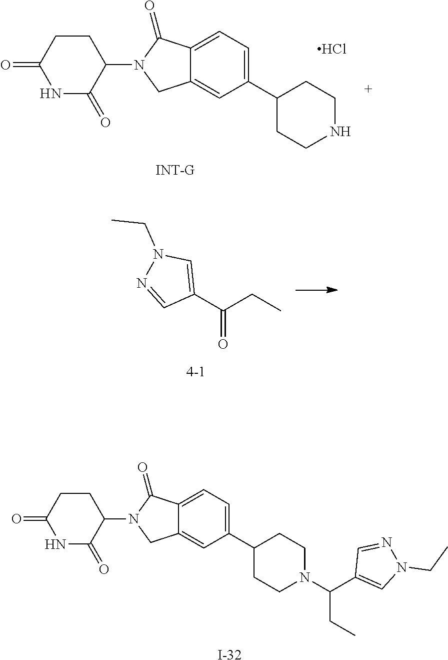

To a stirred solution of INT-G (400 mg, 0.09 mmol) and 4-1 (333 mg, 2.19 mmol) in DMSO (10 mL), was added titanium isopropoxide (993 mg, 3.29 mmol). The resulting mixture was stirred at rt for 16 h and then NaBH₃(CN) (138 mg, 2.20 mmol) was added and stirring was continued at rt for an additional 16 h. Upon complete consumption of the starting materials, the reaction mixture was quenched with aq. NaHCO₃ and extracted with EtOAc (2×75 mL). The combined organic extracts were washed with brine, dried over Na₂SO₄, filtered, and concentrated under reduced pressure. The obtained crude material was purified by silica gel column chromatography eluting with 7% MeOH in DCM to afford I-32 as an off-white solid (170 mg, 0.36 mmol, 34% yield). MS [M+H]=464.3. ¹H NMR (400 MHz, DMSO-d₆): δ 10.75 (s, 1H), 7.65-7.62 (m, 2H), 7.44 (s, 1H), 7.37-7.35 (m, 2H), 5.06 (dd, J=13.2, 4.8 Hz, 1H), 4.42 (d, J=17.2 Hz, 1H), 4.32 (d, J=17.2 Hz, 1H), 4.15-4.10 (m, 2H), 3.68-3.85 (m, 1H), 3.20-3.19 (m, 3H), 2.93-2.84 (m, 2H), 2.67-2.09 (m, 4H), 2.04-1.82 (m, 2H), 1.90-1.65 (m, 4H), 1.39 (t, J=7.6 Hz, 3H), 0.86 (t, J=7.2 Hz, 3H).

### Example 5: 3-(1-oxo-5-(1-(1-(pyrazin-2-yl)propyl)piperidin-4-yl)isoindolin-2-yl)piperidine-2,6-dione (I-33)

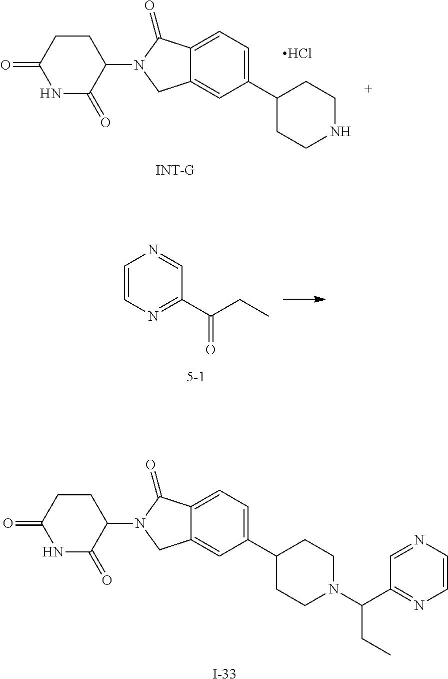

To a stirred solution of INT-G (400 mg, 0.09 mmol) and 5-1 (298 mg, 1.34 mmol) in DMSO (15 mL), titanium isopropoxide (1.24 g, 4.38 mmol) was added. The resulting mixture was stirred at rt for 16 h and then NaBH₃(CN) (137 mg, 2.20 mmol) was added and stirring was continued at rt for an additional 16 h. Upon complete consumption of the starting materials, the reaction mixture was quenched with aq. NaHCO₃ and extracted with EtOAc (2×75 mL). The combined organic extracts were washed with brine, dried over Na₂SO₄, filtered, and concentrated under reduced pressure. The obtained crude material was purified by silica gel column chromatography eluting with 7% MeOH in DCM to afford I-33 as an off-white solid (100 mg, 0.22 mmol, 20% yield). MS [M+H]=448.1. ¹H NMR (400 MHz, DMSO-d₆): δ 10.98 (s, 1H), 8.65-8.63 (m, 2H), 8.54 (d, J=2.4 Hz, 1H), 7.61 (d, J=7.6 Hz, 1H), 7.45 (s, 1H), 7.35 (d, J=8.4 Hz, 1H), 5.09 (dd, J=13.2, 4.8 Hz, 1H), 4.40 (d, J=17.2 Hz, 1H), 4.26 (d, J=17.2 Hz, 1H), 3.69-3.65 (m, 1H), 3.06-2.87 (m, 3H), 3.60-2.35 (m, 4H), 2.08-1.92 (m, 5H), 1.73-1.61 (m, 3H), 0.78 (t, J=7.6 Hz, 3H).

### Example 6: 3-(5-(1-(6,7-dihydro-5H-cyclopenta[b]pyridin-5-yl)piperidin-4-yl)-1-oxoisoindolin-2-yl)piperidine-2,6-dione (I-35)

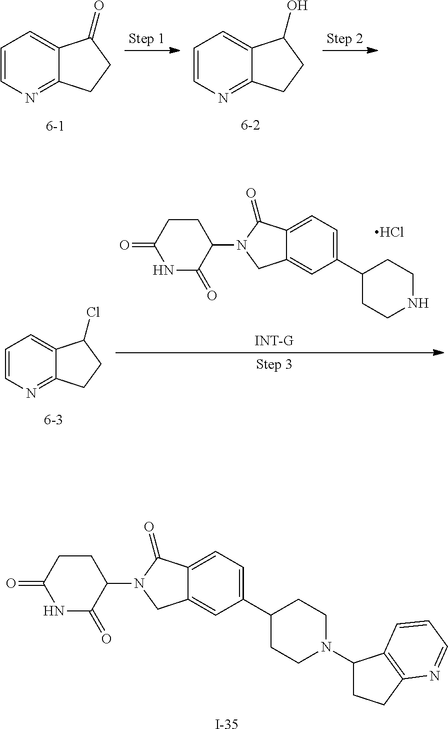

### Step 1. 6,7-dihydro-5H-cyclopenta[b]pyridin-5-ol (6-2)

To a solution of 6-1 (1.0 g, 7.5 mmol) in MeOH (20 mL) was added NaBH₄ (541 mg, 14.3 mmol) in small portions at 0° C. and the resulting mixture was stirred at rt for 2 h. Upon complete consumption of the starting materials, the reaction mixture was quenched with water and extracted with 10% MeOH in DCM (3×75 mL). The combined organic extracts were dried over Na₂SO₄, filtered, and concentrated under reduced pressure. The obtained crude material was purified by silica gel column chromatography eluting with 7% MeOH in DCM to afford compound 6-2 (750 mg, 5.55 mmol, 74% yield). ¹H NMR (400 MHz, CDCl₃): δ 8.33-8.31 (m, 1H), 7.69-7.66 (m, 1H), 7.11-7.07 (m, 1H), 5.27-5.21 (m, 1H), 3.05-2.98 (m, 1H), 2.86-2.81 (m, 1H), 2.51-2.48 (m, 1H), 1.97-1.93 (m, 1H).

### Step 2. 5-chloro-6,7-dihydro-5H-cyclopenta[b]pyridine (6-3)

To a solution of 6-2 (750 mg, 5.55 mmol) in DCM (30 mL) was added SOCl₂ (5 mL) drop wise at 0° C. and the resulting mixture was stirred at 50° C. for 4 h. Upon complete consumption of the starting materials, the reaction mixture was cooled to rt, diluted with DCM (20 mL), and washed with aq. NaHCO₃ (2×50 mL). The combined organic extracts were dried over Na₂SO₄, filtered, and concentrated under reduced pressure to afford crude 6-3 (750 mg), which was used in the next step without further purification. MS [M+H]⁺=153.95.

### Step 3. 3-(5-(1-(6,7-dihydro-5H-cyclopenta[b]pyridin-5-yl)piperidin-4-yl)-1-oxoisoindolin-2-yl)piperidine-2,6-dione (I-35)

To a solution of INT-G (500 mg, 1.52 mmol) in DMF (10 mL) was added Et3N (0.64 mL, 4.6 mmol) followed by crude 6-3 (350 mg) in DCM (2 mL) at 0° C. and the resulting mixture was stirred at 80° C. for 16 h. Upon complete consumption of the starting materials, the reaction mixture was quenched with water and extracted with DCM (3×75 mL). The combined organic extracts were washed with brine, dried over Na₂SO₄, filtered, and concentrated under reduced pressure. The obtained crude material was purified by silica gel column chromatography eluting with 8% MeOH in DCM to afford I-35 as brown colored solid (200 mg, 0.45 mmol, 30% yield). MS [M+H]⁺=445.2. ¹H NMR (400 MHz, DMSO-d₆): δ 10.75 (s, 1H), 10.05 br s, 1H), 8.48 (br s, 1H), 7.66 (d, J=7.6 Hz, 1H), 7.46 (s, 1H), 7.39 (d, J=7.6 Hz, 1H), 7.26 (br s, 1H), 5.05 (dd, J=13.2, 4.8 Hz, 1H), 4.44 (d, J=17.2 Hz, 1H), 4.33 (d, J=17.2 Hz, 1H), 3.1-3.02 (m, 5H), 2.94-2.85 (m, 3H), 2.67-2.34 (m, 2H), 2.05-2.00 (m, 3H), 1.91-1.21 (m, 5H).

### Example 7: 3-(5-(1-(4-methoxycyclohexyl)piperidin-4-yl)-1-oxoisoindolin-2-yl)piperidine-2,6-dione (I-36)

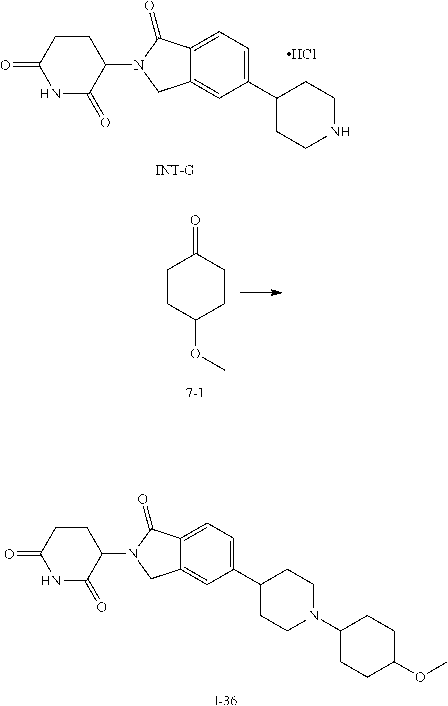

To a stirred solution of INT-G (450 mg, 1.23 mmol) and 7-1 (517 mg, 3.69 mmol) in DMSO (15 mL) was added titanium isopropoxide (698 mg, 2.46 mmol) was added. The resulting mixture was stirred at rt for 16 h and then NaBH(OAc)₃ (1.3 g, 6.15 mmol) was added and stirring was continued at rt for an additional 16 h. Upon complete consumption of the starting materials, the reaction mixture was quenched with aq. NaHCO₃ and extracted with EtOAc (2×75 mL). The combined organic extracts were washed with brine, dried over Na₂SO₄, filtered, and concentrated under reduced pressure. The obtained crude material was purified by silica gel column chromatography eluting with 7% MeOH in DCM to afford 1-36 as an off-white solid (100 mg, 0.23 mmol, 19% yield). MS [M+H]=440.3. ¹H NMR (400 MHz, DMSO-d₆): δ 10.99 (s, 1H), 7.63 (d, J=8.0 Hz, 1H), 7.48 (s, 1H), 7.39 (d, J=7.6 Hz, 1H), 5.09 (dd, J=13.2, 4.8 Hz, 1H), 4.42 (d, J=17.2 Hz, 1H), 4.27 (d, J=17.2 Hz, 1H), 3.20 (s, 3H), 2.94-2.88 (m, 4H), 2.67-2.37 (m, 6H), 2.05-1.72 (m, 6H), 1.68-1.23 (m, 7H).

### Example 8: 3-(5-(1-(((1r,4r)-4-(fluoromethyl)cyclohexyl)methyl)piperidin-4-yl)-1-oxoisoindolin-2-yl)piperidine-2,6-dione trifluoroacetate (I-10) and 3-(5-(1-(((S)-4-methylcyclohex-3-en-1-yl)methyl)piperidin-4-yl)-1-oxoisoindolin-2-yl)piperidine-2,6-dione trifluoroacetate (I-14)

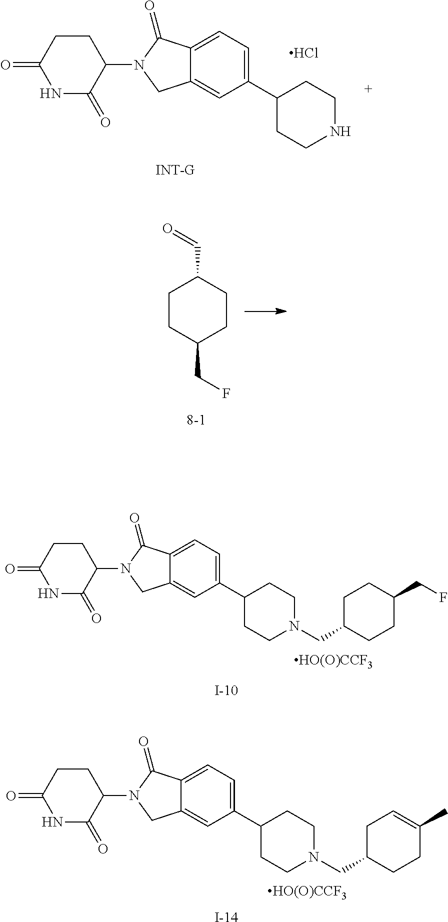

To a solution of INT-G (150 mg, 0.41 mmol) and 8-1 (89 mg, 0.62 mmol) in DMF (7.5 mL), was added NaBH(OAc)₃ (261 mg, 1.23 mmol) at 0° C. and the resulting mixture was stirred at rt for 16 h. Upon complete consumption of the starting materials, the reaction mixture was quenched with ice-cold water and extracted with EtOAc (2×50 mL). The combined organic extracts were washed with brine, dried over Na₂SO₄ and concentrated under reduced pressure. The obtained crude material was purified by silica gel column chromatography eluting with 5% MeOH in DCM. The obtained material was further purified by prep HPLC (ZORBAX XDB C18, (21.2 mm×150 mm), Mobile phase: 0.1% TFA in H₂O (A): MeCN (B), Flow: 20 mL/mm, Time/% B: 0/20, 2/30, 9/40) to afford I-10 as an off-white solid (5 mg, 0.01 mmol, 6% yield, TFA salt) and I-14 as an off-white solid (22 mg, 0.05 mmol, 23% yield, TFA salt). I-10: MS [M+H]⁺=436.4. ¹H NMR (400 MHz, DMSO-d₆): δ 10.98 (s, 1H), 7.68 (d, J=7.6 Hz, 1H), 7.44 (s, 1H), 7.36 (d, J=7.6 Hz, 1H), 5.08 (dd, J=13.2, 4.8 Hz, 1H), 4.44-4.40 (m, 2H), 4.30-4.26 (m, 2H), 4.18-4.16 (m, 1H), 3.59-3.56 (m, 2H), 3.16-2.85 (m, 6H), 2.64-2.46 (m, 2H), 1.98-1.60 (m, 10H), 1.02-0.96 (m, 4H). I-14: MS [M+H]⁺=456.4. ¹H NMR (400 MHz, DMSO-d₆): δ 10.99 (s, 1H), 7.63 (d, J=7.6 Hz, 1H), 7.49 (s, 1H), 7.39 (d, J=8.0 Hz, 1H), 5.34 (br s, 1H), 5.08 (dd, J=13.2, 4.8 Hz, 1H), 4.42 (d, J=17.2 Hz, 1H), 4.28 (d, J=17.2 Hz, 1H), 2.95-2.91 (m, 3H), 2.61-2.18 (m, 5H), 1.99-1.71 (m, 13H), 1.60 (s, 3H), 1.21-1.19 (m, 1H).

### Example 9: 3-(5-(1-((4-methylpyrimidin-5-yl)methyl)piperidin-4-yl)-1-oxoisoindolin-2-yl)piperidine-2,6-dione (I-12)

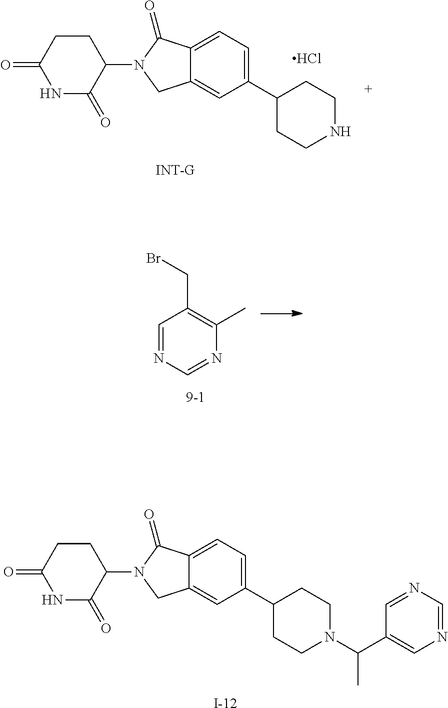

To a stirred solution of INT-G 150 mg, 0.41 mmol) and Et3N (0.18 mL, 2.06 mmol) in DMF (5 mL), was added 9-1 (85 mg, 0.45 mmol) in DMF (5 mL) was added at 0° C. and the resulting mixture was stirred at rt for 16 h. Upon complete consumption of the starting materials, the reaction mixture was quenched with ice-cold water and extracted with EtOAc (2×50 mL).

The combined organic extracts were washed with brine, dried over Na₂SO₄, filtered, and concentrated under reduced pressure. The obtained crude material was purified by silica gel column chromatography eluting with 8% MeOH in DCM to afford I-12 as an off-white solid (75 mg, 0.17 mmol, 42% yield). MS [M+H]=434.4. ¹H NMR (400 MHz, DMSO-d₆): δ 10.98 (s, 1H), 8.93 (s, 1H), 8.55 (s, 1H), 7.63 (d, J=7.6 Hz, 1H), 7.50 (s, 1H), 7.40 (d, J=8.0 Hz, 1H), 5.09 (dd, J=13.2, 4.8 Hz, 1H), 4.42 (d, J=17.2 Hz, 1H), 4.27 (d, J=17.2 Hz, 1H), 3.53 (s, 2H), 2.94-2.87 (m, 3H), 2.69-2.32 (m, 6H), 2.19-2.13 (m, 2H), 2.00-1.96 (m, 1H), 1.80-1.66 (m, 4H).

### Example 10: 3-(1-oxo-5-(1-(pyridazin-3-ylmethyl)piperidin-4-yl)isoindolin-2-yl)piperidine-2,6-dione (I-15)

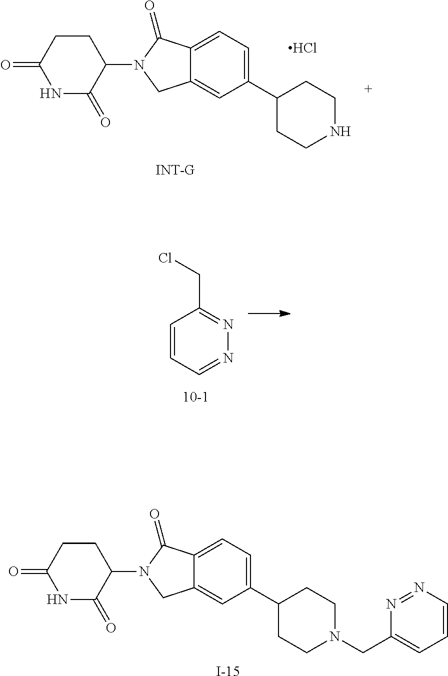

To a stirred solution of INT-G (250 mg, 0.68 mmol) and Et3N (0.18 mL, 2.06 mmol) in DMF (7 mL) was added 10-1 (106 mg, 0.82 mmol), dissolved in DMF (5 mL), at 0° C. and the resulting mixture was stirred at rt for 16 h. Upon complete consumption of the starting materials, the reaction mixture was quenched with ice-cold water and extracted with EtOAc (2×75 mL). The combined organic extracts were washed with brine, dried over Na₂SO₄, filtered, and concentrated under reduced pressure. The obtained crude material was purified by silica gel column chromatography eluting with 10% MeOH in DCM to afford I-15 as pale brown solid (110 mg, 0.26 mmol, 36% yield). MS [M+H]=420.1. ¹H NMR (400 MHz, DMSO-d₆): δ 10.99 (s, 1H), 9.15-9.13 (m, 1H), 7.76-7.63 (m, 3H), 7.49 (s, 1H), 7.40 (d, J=8.0 Hz, 1H), 5.10 (dd, J=13.2, 4.8 Hz, 1H), 4.42 (d, J=17.2 Hz, 1H), 4.28 (d, J=17.2 Hz, 1H), 3.84 (s, 2H), 2.93-2.90 (m, 3H), 2.67-2.32 (m, 3H), 2.24-2.18 (m, 2H), 2.01-1.98 (m, 1H), 1.71-1.65 (m, 4H).

### Example 11: 3-(1-oxo-5-(1-(pyrazin-2-ylmethyl)piperidin-4-yl)isoindolin-2-yl)piperidine-2,6-dione (I-14)

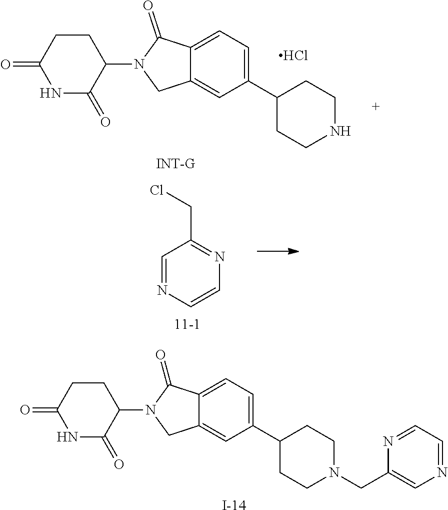

To a stirred solution of INT-G (250 mg, 0.68 mmol) and Et3N (0.18 mL, 2.06 mmol) in DMF (7 mL) was added 11-1 (140 mg, 0.82 mmol), dissolved in DMF (5 mL), at 0° C. and the resulting mixture was stirred at rt for 16 h. Upon complete consumption of the starting materials, the reaction mixture was quenched with ice-cold water and extracted with EtOAc (2×75 mL).

The combined organic extracts were washed with brine, dried over Na₂SO₄, filtered, and concentrated under reduced pressure. The obtained crude material was purified by silica gel column chromatography eluting with 10% MeOH in DCM to afford I-14 as an off-white solid (120 mg, 0.28 mmol, 42% yield). MS [M+H]=420.3. ¹H NMR (400 MHz, DMSO-d₆): δ 10.99 (s, 1H), 8.72 (d, J=1.2 Hz, 1H), 8.60-8.58 (m, 1H), 8.54 (d, J=2.4 Hz, 1H), 7.64 (d, J=8.0 Hz, 1H), 7.50 (s, 1H), 7.41 (d, J=8.4 Hz, 1H), 5.10 (dd, J=13.2, 4.8 Hz, 1H), 4.41 (d, J=17.2 Hz, 1H), 4.28 (d, J=17.2 Hz, 1H), 3.71 (s, 2H), 2.98-2.92 (m, 3H), 2.67-2.32 (m, 3H), 2.21-2.19 (m, 2H), 2.01-1.98 (m, 1H), 1.78-1.75 (m, 4H).

### Example 12: 3-(1-oxo-5-(1-(pyrimidin-4-ylmethyl)piperidin-4-yl)isoindolin-2-yl)piperidine-2,6-dione (I-16)

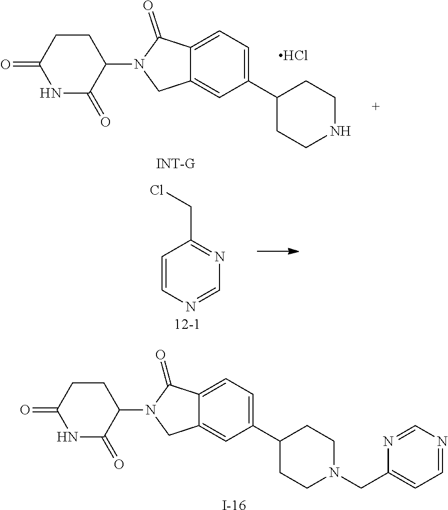

To a stirred solution of INT-G (250 mg, 0.68 mmol) and Et3N (0.18 mL, 2.06 mmol) in DMF (7 mL) was added 12-1 (89 mg, 0.68 mmol), dissolved in DMF (5 mL), at 0° C. and the resulting mixture was stirred at rt for 16 h. Upon complete consumption of the starting materials, the reaction mixture was quenched with ice-cold water and extracted with EtOAc (2×75 mL). The combined organic extracts were washed with brine, dried over Na₂SO₄, filtered, and concentrated under reduced pressure. The obtained crude material was purified by silica gel column chromatography eluting with 8% MeOH in DCM to afford I-16 as an off-white solid (150 mg, 0.36 mmol, 52% yield). MS [M+H]=420.1. ¹H NMR (400 MHz, DMSO-d₆): δ 10.99 (s, 1H), 9.10 (d, J=1.2 Hz, 1H), 8.77-8.76 (m, 1H), 7.66-7.60 (m, 2H), 7.51 (s, 1H), 7.42 (d, J=8.0 Hz, 1H), 5.10 (dd, J=13.2, 4.8 Hz, 1H), 4.43 (d, J=17.2 Hz, 1H), 4.29 (d, J=17.2 Hz, 1H), 3.65 (s, 2H), 2.96-2.87 (m, 3H), 2.68-235 (m, 3H), 2.25-2.19 (m, 2H), 2.05-1.98 (m, 1H), 1.80-1.75 (m, 4H).

### Example 13: 3-(1-oxo-5-(1-(pyridazin-4-ylmethyl)piperidin-4-yl)isoindolin-2-yl)piperidine-2,6-dione (I-18)

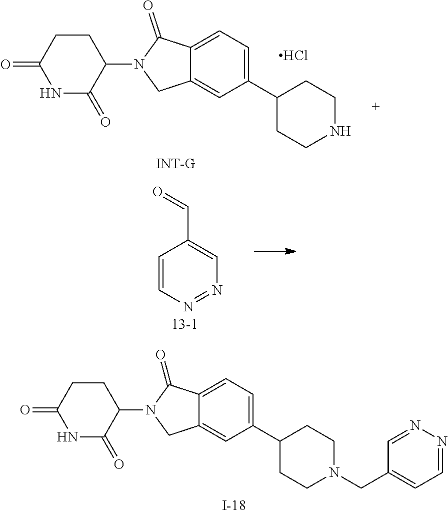

To a stirred solution of INT-G (250 mg, 0.68 mmol) and 13-1 (147 mg, 1.36 mmol) in DMF (10 mL), was added NaBH(OAc)₃ (432 mg, 2.04 mmol) at 0° C. and the resulting mixture was stirred at 60° C. for 16 h. Upon complete consumption of the starting materials, the reaction mixture was cooled to rt, quenched with ice-cold water and extracted with EtOAc (2×75 mL). The combined organic extracts were washed with brine, dried over Na₂SO₄, filtered, and concentrated under reduced pressure. The obtained crude material was purified by silica gel column chromatography eluting with 5% MeOH in DCM to afford I-18 as an off-white solid (74 mg, 0.18 mmol, 27% yield). MS [M+H]=420.2. ¹H NMR (400 MHz, DMSO-d₆): δ 10.98 (s, 1H), 9.21 (s, 1H), 9.18-9.15 (m, 1H), 7.65-7.62 (m, 2H), 7.50 (s, 1H), 7.40 (d, J=8.0 Hz, 1H), 5.10 (dd, J=13.2, 4.8 Hz, 1H), 4.42 (d, J=17.2 Hz, 1H), 4.28 (d, J=17.2 Hz, 1H), 3.61 (s, 2H), 2.88-2.87 (m, 3H), 2.67-2.32 (m, 3H), 2.16-2.12 (m, 2H), 1.99-1.95 (m, 1H), 1.78-1.76 (m, 4H).

### Example 14: 3-(5-(1-((2-methylpyrimidin-5-yl)methyl)piperidin-4-yl)-1-oxoisoindolin-2-yl)piperidine-2,6-dione (I-17)

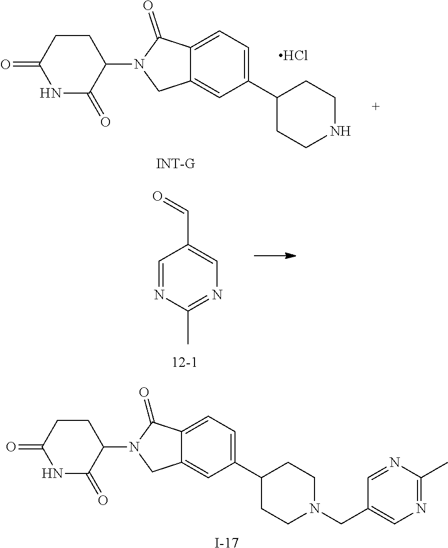

To a stirred solution of INT-G (250 mg, 0.68 mmol) and 12-1 (147 mg, 1.36 mmol) in DMF (10 mL), was added NaBH(OAc)₃ (432 mg, 2.04 mmol) at 0° C. and the resulting mixture was stirred at 60° C. for 16 h. Upon complete consumption of the starting materials, the reaction mixture was cooled to rt, quenched with ice-cold water and extracted with EtOAc (2×75 mL). The combined organic extracts were washed with brine, dried over Na₂SO₄, filtered, and concentrated under reduced pressure. The obtained crude material was purified by silica gel column chromatography eluting with 5% MeOH in DCM to afford I-17 as an off-white solid (74 mg, 0.18 mmol, 27% yield). MS [M+H]=434.1. ¹H NMR (400 MHz, DMSO-d₆): δ 10.98 (s, 1H), 8.62 (s, 2H), 7.63 (d, J=8.0 Hz, 1H), 7.49 (s, 1H), 7.38 (d, J=8.0 Hz, 1H), 5.10 (dd, J=13.2, 4.8 Hz, 1H), 4.42 (d, J=17.2 Hz, 1H), 4.28 (d, J=17.2 Hz, 1H), 3.52 (s, 2H), 2.92-2.85 (m, 3H), 2.65-2.58 (m, 2H), 2.60 (s, 3H), 2.48-2.38 (m, 1H), 2.12-1.96 (m, 3H), 1.75-1.65 (m, 4H).

### Example 15: 3-(1-oxo-5-(1-((6-oxo-1,6-dihydropyridin-3-yl)methyl)piperidin-4-yl)isoindolin-2-yl)piperidine-2,6-dione (I-1)

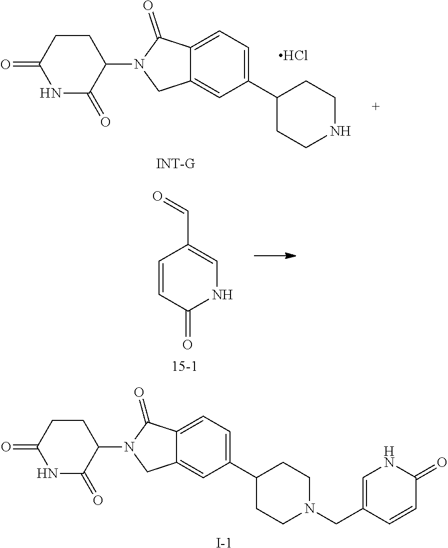 [SMILES: O=C1CCC(N2Cc3cc(C4CCNCC4)ccc3C2=O)C(=O)N1 ; Cl ; O=Cc1ccc(=O)[nH]c1 ; O=C1CCC(N2Cc3cc(C4CCN(Cc5ccc(=O)[nH]c5)CC4)ccc3C2=O)C(=O)N1 ; [I-]]

To a stirred solution of INT-G (150 mg, 0.41 mmol) and 15-1 (76 mg, 0.62 mmol) in DMF (10 mL) was added NaBH(OAc)₃ (262 mg, 1.23 mmol) at 0° C. and the resulting mixture was stirred at 60° C. for 16 h. Upon complete consumption of the starting materials, the reaction mixture was cooled to rt, quenched with ice-cold water and extracted with EtOAc (2×75 mL). The combined organic extracts were washed with brine, dried over Na₂SO₄, filtered, and concentrated under reduced pressure. The obtained crude material was purified by silica gel column chromatography eluting with 5% MeOH in DCM to afford I-1 as an off-white solid (70 mg, 0.16 mmol, 39% yield). MS [M+H]=435.2. ¹H NMR (400 MHz, DMSO-d₆): δ 11.45 (s, 1H), 10.99 (s, 1H), 7.63 (d, J=8.0 Hz, 1H), 7.49 (s, 1H), 7.42-7.38 (m, 2H), 7.22 (s, 1H), 6.31 (d, J=9.2 Hz, 1H), 5.10 (dd, J=13.2, 4.8 Hz, 1H), 4.41 (d, J=17.2 Hz, 1H), 4.27 (d, J=17.2 Hz, 1H), 3.23 (s, 2H), 2.91-2.89 (m, 3H), 2.65-2.55 (m, 3H), 2.41-2.38 (m, 1H), 2.05-1.98 (m, 2H), 1.78-1.65 (m, 4H).

### Example 16: 3-(1-oxo-5-(1-((2-oxo-1,2-dihydropyridin-3-yl)methyl)piperidin-4-yl)isoindolin-2-yl)piperidine-2,6-dione (I-38)

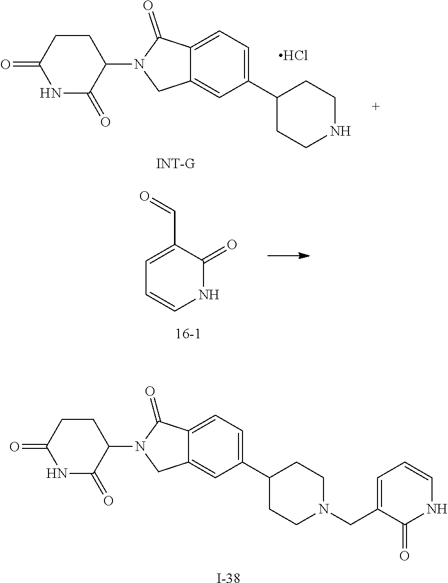

To a stirred solution of INT-G (150 mg, 0.41 mmol) and 16-1 (152 mg, 1.23 mmol) in DMF (10 mL), was added NaBH(OAc)₃ (261 mg, 1.23 mmol) at 0° C. and the resulting mixture was stirred at 60° C. for 16 h. Upon complete consumption of the starting materials, the reaction mixture was cooled to rt, quenched with ice-cold water and extracted with EtOAc (2×75 mL). The combined organic extracts were washed with brine, dried over Na₂SO₄, filtered, and concentrated under reduced pressure. The obtained crude material was purified by silica gel column chromatography eluting with 5% MeOH in DCM to afford I-38 as an off-white solid (50 mg, 0.11 mmol, 28% yield). MS [M+H]=435.3. ¹H NMR (400 MHz, DMSO-d₆): δ 11.55 (s, 1H), 10.99 (s, 1H), 7.64 (d, J=8.0 Hz, 1H), 7.50 (s, 1H), 7.47-7.38 (m, 2H), 2.28-7.22 (m, 1H), 6.21-6.18 (m, 1H), 5.10 (dd, J=13.2, 4.8 Hz, 1H), 4.41 (d, J=17.2 Hz, 1H), 4.28 (d, J=17.2 Hz, 1H), 2.98-2.85 (m, 3H), 2.68-2.55 (m, 2H), 2.48-2.32 (m, 2H), 2.15-1.98 (m, 4H), 1.70-1.65 (m, 4H).

### Example 17: 3-(5-(2,6-dimethylpiperidin-4-yl)-1-oxoisoindolin-2-yl)piperidine-2,6-dione hydrochloride (I-4)

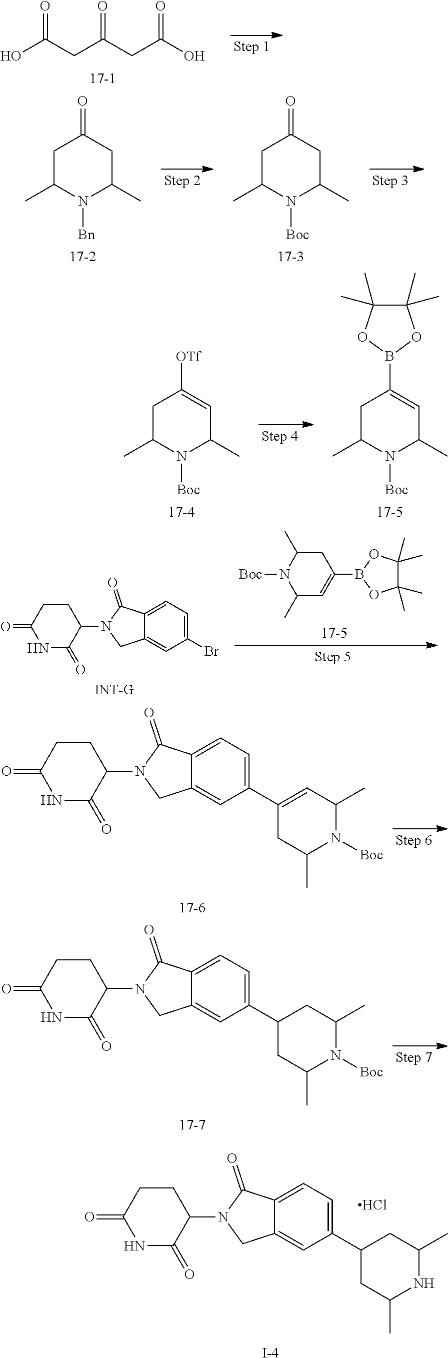

### Step 1. 1-benzyl-2,6-dimethylpiperidin-4-one (17-2)

To a solution of 17-1 (10.0 g, 68.5 mmol) and acetaldehyde (7.73 mL, 137 mmol) in water (50 mL) was added benzyl amine (7.48 mL, 68.5 mmol) dropwise at 10° C. (vigorous gas evolution was observed) and stirred at rt for 72 h. After completion of the reaction, pH was adjusted to 2 by adding 1M aq. HCl and stirred at rt. After 1 h, pH was neutralized with sat. aq. NaHCO₃ and extracted with DCM (3×150 mL). The combined organic extracts were washed with brine, dried over Na₂SO₄ and concentrated under reduced pressure. The obtained crude material was purified by silica gel column chromatography eluting with 20% EtOAc in hexanes to afford 17-2 (4.1 g, 18.87 mmol, 27% yield) as an off-white solid. MS [M+H]=218.1.

### Step 2. tert-butyl 2,6-dimethyl-4-oxopiperidine-1-carboxylate (17-3)

To a solution of 17-2 (1.2 g, 5.5 mmol) and Boc₂O (2.43 mL, 11.0 mmol) in EtOH (50 mL) was added 10% Pd/C (300 mg) and stirred under hydrogen atmosphere for 24 h in parr apparatus. After completion of the reaction, the reaction mixture was filtered through a small pad of Celite® was washed with EtOH. The combined filtrates were concentrated to dryness and the obtained crude material was purified by silica gel column chromatography eluting with 15% EtOAc in hexanes to afford 17-3 (850 mg, 3.74 mmol, 68% yield) as white solid. ¹H NMR (300 MHz, CDCl₃): δ 4.40-4.35 (m, 2H), 2.88-2.80 (m, 2H), 2.40-2.33 (m, 2H), 1.49 (s, 9H), 1.24 (d, J=6.6 Hz, 6H).

### Step 3. tert-butyl 2,6-dimethyl-4-(((trifluoromethyl)sulfonyl)oxy)-3,6-dihydropyridine-1(2H)-carboxylate (17-4)

To a solution of 17-3 (600 mg, 2.64 mmol) in THF (25 mL) was added LiHMDS (3.17 mL, 3.17 mmol, 1M in THF) dropwise at −78° C. and the obtained reaction mixture was stirred at the same temperature for 1 h. N-(5-chloropyridin-2-yl)-1,1,1-trifluoro-N-((trifluoromethyl)sulfonyl)methanesulfonamide (1.34 g, 3.43 mmol), dissolved in THF (10 mL), was added dropwise and the reaction mixture was stirred at 0° C. for 3 h. The reaction mixture was quenched with sat. aq. NH₄Cl and extracted with EtOAc (3×50 mL). The combined organic extracts were washed with brine, dried over Na₂SO₄ and concentrated to dryness. The obtained crude material was purified by silica gel column chromatography eluting with 10% EtOAc in hexanes to afford 17-4 (500 mg, 1.39 mmol, 53% yield) as pale-yellow liquid. ¹H NMR (300 MHz, CDCl₃): δ 5.80-5.78 (m, 1H), 4.39-4.30 (m, 2H), 2.88-2.80 (m, 1H), 2.20-2.13 (m, 1H), 1.48 (s, 9H), 1.36 (d, J=5.8 Hz, 3H), 1.23 (d, J=6.3 Hz, 3H).

### Step 4. tert-butyl 2,6-dimethyl-4-(4,4,5,5-tetramethyl-1,3,2-dioxaborolan-2-yl)-3,6-dihydropyridine-1(2H)-carboxylate (17-5)

To a solution of 17-4 (500 mg, 1.39 mmol) in dioxane (20 mL) were added bis(pinacolato)diboron (389 mg, 1.53 mmol) and KOAc (409 mg, 4.17 mmol) and degassed for 10 min with argon. Then, PdCl₂(dppf)·DCM (56 mg, 0.07 mmol) and dppf (38 mg, 0.07 mmol) were added and the reaction mixture was stirred at 90 ^{e}C for 16 h. The reaction mixture was allowed to cool to rt, filtered through a small pad of Celite® was washed with dioxane. The combined filtrates were evaporated under reduced pressure to afford crude 17-5 (950 mg) as an off-white solid, which was used in the next step without further purification. ¹H NMR (400 MHz, CDCl₃): δ 6.58-6.55 (m, 1H), 4.25-4.15 (m, 2H), 2.40-2.35 (m, 1H), 2.19-2.14 (m, 1H), 1.47 (s, 6H), 1.27-1.24 (m, 21H).

### Step 5. tert-butyl 4-(2-(2,6-dioxopiperidin-3-yl)-1-oxoisoindolin-5-yl)-2,6-dimethyl-3,6-dihydropyridine-1(2H)-carboxylate (17-6)

To a solution of INT-G (500 mg, 1.54 mmol) and crude 17-5 (940 mg) in DMF (10 mL), was added K₂CO₃ (640 mg, 4.64 mmol) and the resulting mixture was degassed for 15 min with argon. Then, PdCl₂(dppf)·DCM (94 mg, 0.11 mmol) was then added and the reaction mixture was stirred at 100° C. for 16 h. The reaction mixture was cooled to rt and filtered through a small pad of Celite® and washed with DMF (20 mL). Ice-cold water was added to the filtrate and the obtained solid was filtered. The obtained crude material was purified by silica gel column chromatography eluting with 5% MeOH in DCM to afford 17-6 as an off-white solid (530, 1.17 mmol, 75% yield). MS [M+H]⁺=454.4.

### Step 6. tert-butyl 4-(2-(2,6-dioxopiperidin-3-yl)-1-oxoisoindolin-5-yl)-2,6-dimethylpiperidine-1-carboxylate (17-7)

To a solution of 17-6 (530 mg, 1.17 mmol) in DMF (20 mL) was added Pd/C (10 wt %, 200 mg) and the resulting mixture was stirred at rt under an atmosphere of hydrogen (in parr apparatus, 60 psi) for 16 h. Upon complete consumption of the starting material, the reaction mixture was filtered through a pad of Celite® and washed with DMF. The filtrate was quenched with water and extracted with EtOAc (2×100 mL). The combined organic extracts were washed with brine (50 mL), dried over Na₂SO₄, filtered, and concentrated under reduced pressure to afford 17-7 as an off-white solid (290 mg, 0.64 mmol, 55% yield). MS [M-tBu+H]⁺=400.1.

### Step 7. 3-(5-(2,6-dimethylpiperidin-4-yl)-1-oxoisoindolin-2-yl)piperidine-2,6-dione hydrochloride (I-4)

To a solution of 17-7 (290 mg, 0.64 mmol) in dioxane (10 mL) was added drop wise 4M HCl in dioxane (3 ml) at 0° C. and the resulting mixture was stirred at rt for 16 h. Upon complete consumption of the starting materials, the solvent was evaporated under reduced pressure. The crude material was triturated with diethyl ether and dried under reduced pressure to afford 1-4 as an off-white solid (150 mg, 0.38 mmol, 60% yield). MS [M+H]⁺=356.2. ¹H NMR (400 MHz, DMSO-d₆): δ 11.00 (s, 1H), 8.82 (br s, 1H), 7.70 (d, J=8.0 Hz, 1H), 7.47 (s, 1H), 7.38 (d, J=8.8 Hz, 1H), 5.11 (dd, J=13.2, 4.8 Hz, 1H), 4.44 (d, J=17.2 Hz, 1H), 4.31 (d, J=17.2 Hz, 1H), 3.79-3.78 (m, 1H), 3.50-3.47 (m, 1H), 3.27-3.21 (m, 1H), 2.96-2.87 (m, 1H), 2.67-2.50 (m, 1H), 2.41-2.34 (m, 1H), 2.07-1.94 (m, 3H), 1.80-1.77 (m, 1H), 1.66-1.60 (m, 1H), 1.42 (d, J=6.8 Hz, 3H), 1.27 (d, J=6.8 Hz, 3H).

### Example 18: 3-(5-(1-benzyl-2,6-dimethylpiperidin-4-yl)-1-oxoisoindolin-2-yl)piperidine-2,6-dione hydrochloride (I-2)

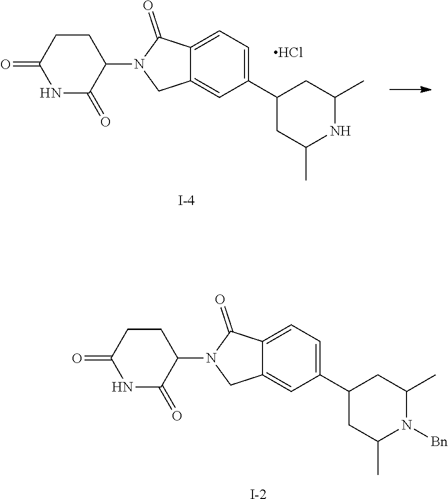

To a stirred solution of I-4 (100 mg, 0.25 mmol) and Et3N (0.11 mL, 0.76 mmol) in DMF (5 mL) was added benzyl bromide (0.04 mL, 0.30 mmol) at 0° C. and the resulting mixture was stirred at rt for 16 h. Upon complete consumption of the starting materials, the reaction mixture was quenched with ice-cold water and extracted with EtOAc (2×50 mL). The combined organic extracts were washed with brine, dried over Na₂SO₄, filtered, and concentrated under reduced pressure. The obtained crude material was purified by silica gel column chromatography eluting with 6% MeOH in DCM to afford I-2 as an off-white solid (30 mg, 0.07 mmol, 26% yield). MS [M+H]⁺=446.1. ¹H NMR (400 MHz, DMSO-d₆): δ 10.99 (s, 1H), 7.65 (d, J=7.6 Hz, 1H), 7.51 (s, 1H), 7.41-7.20 (m, 6H), 5.11 (dd, J=13.4, 4.8 Hz, 1H), 4.45 (d, J=17.2 Hz, 1H), 4.28 (d, J=17.2 Hz, 1H), 3.93-3.89 (m, 1H), 3.48-3.45 (m, 1H), 3.10-2.85 (m, 4H), 2.65-2.35 (m, 2H), 2.03-1.45 (m, 5H), 1.08-1.05 (m, 6H).

### Example 19: 3-(5-(1-((5-methoxypyridin-2-yl)methyl)piperidin-4-yl)-1-oxoisoindolin-2-yl)piperidine-2,6-dione (I-6)

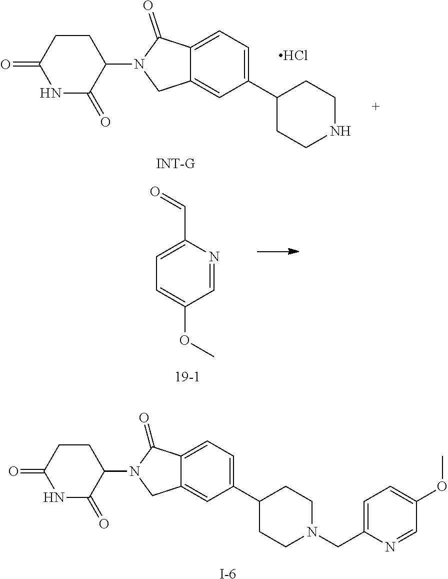

To a stirred solution of INT-G (150 mg, 0.41 mmol) and 19-1 (85 mg, 0.62 mmol) in DMF (10 mL), was added NaBH(OAc)₃ (262 mg, 1.23 mmol) at 0° C. and the resulting mixture was stirred at 60° C. for 16 h. After completion of the reaction, the mixture was allowed to cool to rt, quenched with ice-cold water and extracted with EtOAc (2×75 mL). The combined organic extracts were washed with brine, dried over Na₂SO₄, filtered, and concentrated under reduced pressure. The obtained crude material was purified by silica gel column chromatography eluting with 7% MeOH in DCM to afford I-6 as an off-white solid (40 mg, 0.09 mmol, 22% yield). MS [M+H]⁺=449.2. ¹H NMR (400 MHz, DMSO-d₆): δ 10.99 (s, 1H), 8.20-8.19 (m, 1H), 7.63 (d, J=8.0 Hz, 1H), 7.49 (s, 1H), 7.41-7.35 (m, 3H), 5.10 (dd, J=13.2, 4.8 Hz, 1H), 4.42 (d, J=17.2 Hz, 1H), 4.28 (d, J=17.2 Hz, 1H), 3.81 (s, 3H), 3.56 (s, 2H), 2.94-2.75 (m, 3H), 2.66-2.33 (m, 3H), 2.14-1.96 (m, 3H), 1.77-1.66 (m, 4H).

### Example 20: 3-(5-(1-((5-ethoxypyridin-2-yl)methyl)piperidin-4-yl)-1-oxoisoindolin-2-yl)piperidine-2,6-dione (I-5)

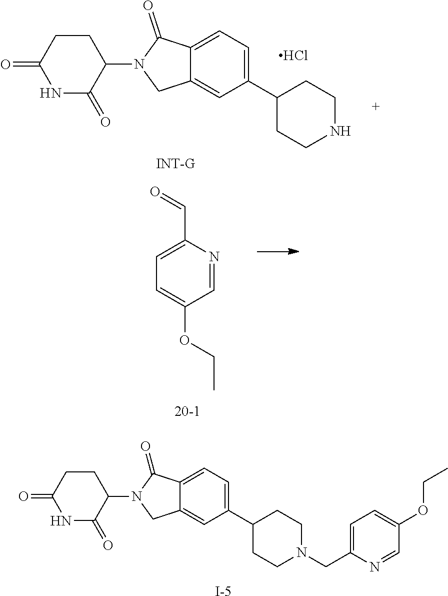

To a stirred solution of INT-G (150 mg, 0.41 mmol) and 20-1 (93 mg, 0.62 mmol) in DMF (10 mL) was added NaBH(OAc)₃ (262 mg, 1.23 mmol) at 0° C. and the resulting mixture was stirred at 60° C. for 16 h. After completion of the reaction, the mixture was allowed to cool to rt, quenched with ice-cold water and extracted with EtOAc (2×75 mL). The combined organic extracts were washed with brine, dried over Na₂SO₄, filtered, and concentrated under reduced pressure. The obtained crude material was purified by silica gel column chromatography eluting with 7% MeOH in DCM to afford I-5 as an off-white solid (60 mg, 0.13 mmol, 32% yield). MS [M+H]⁺=463.2. ¹H NMR (400 MHz, DMSO-d₆): δ 10.99 (s, 1H), 8.18-8.17 (m, 1H), 7.63 (d, J=8.0 Hz, 1H), 7.49 (s, 1H), 7.41-7.33 (m, 3H), 5.09 (dd, J=13.2, 4.8 Hz, 1H), 4.42 (d, J=17.2 Hz, 1H), 4.26 (d, J=17.2 Hz, 1H), 4.08 (q, J=6.8 Hz, 2H), 3.55 (s, 2H), 3.04-2.86 (m, 3H), 2.66-2.33 (m, 3H), 2.14-1.96 (m, 3H), 1.77-1.66 (m, 4H), 1.33 (t, J=6.8 Hz, 3H).

### Example 21: 3-(5-(1-((6-methoxypyridin-3-yl)methyl)piperidin-4-yl)-1-oxoisoindolin-2-yl)piperidine-2,6-dione (1-7)

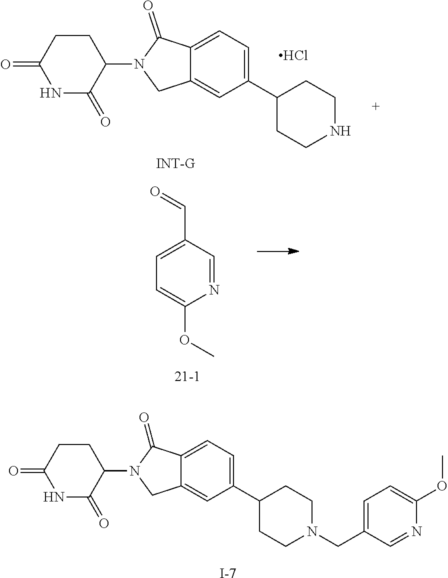

To a stirred solution of INT-G (150 mg, 0.41 mmol) and 21-1 (147 mg, 0.82 mmol) in DMF (10 mL) was added NaBH(OAc)₃ (262 mg, 1.23 mmol) at 0° C. and the resulting mixture was stirred at 60° C. for 16 h. After completion of the reaction, the mixture was allowed to cool to rt, quenched with ice-cold water and extracted with EtOAc (2×50 mL). The combined organic extracts were washed with brine, dried over Na₂SO₄, filtered, and concentrated under reduced pressure. The obtained crude material was purified by silica gel column chromatography eluting with 4% MeOH in DCM to afford I-7 as pale brown solid (70 mg, 0.15 mmol, 34% yield). MS [M+H]⁺=449.1. ¹H NMR (400 MHz, DMSO-d₆): δ 10.99 (s, 1H), 8.07 (s, 1H), 7.66-7.62 (m, 2H), 7.48 (s, 1H), 7.39 (d, J=8.0 Hz, 1H), 6.80 (d, J=8.0 Hz, 1H), 5.09 (dd, J=13.2, 4.8 Hz, 1H), 4.42 (d, J=17.2 Hz, 1H), 4.27 (d, J=17.2 Hz, 1H), 3.83 (s, 3H), 3.45 (br s, 2H), 2.94-2.87 (m, 3H), 2.61-2.32 (m, 3H), 2.05-1.98 (m, 3H), 1.79-1.67 (m, 4H).

### Example 22: 3-(5-(1-((6-ethoxypyridin-3-yl)methyl)piperidin-4-yl)-1-oxoisoindolin-2-yl)piperidine-2,6-dione (I-8)

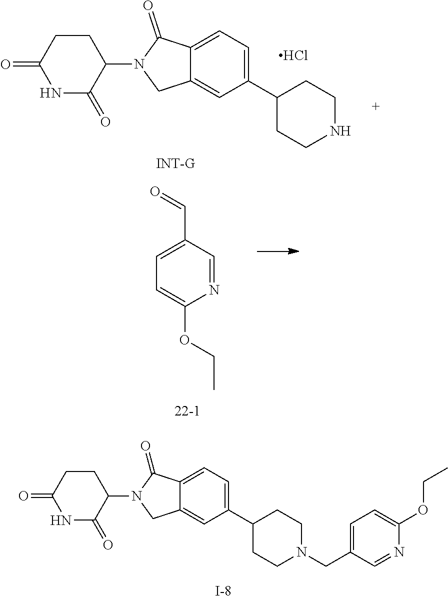

To a stirred solution of INT-G (150 mg, 0.41 mmol) and 22-1 (93 mg, 0.62 mmol) in DMF (10 mL) was added NaBH(OAc)₃ (262 mg, 1.23 mmol) at 0° C. and the resulting mixture was stirred at 60° C. for 16 h. After completion of the reaction, the mixture was allowed to cool to rt, quenched with ice-cold water and extracted with EtOAc (2×50 mL). The combined organic extracts were washed with brine, dried over Na₂SO₄, filtered, and concentrated under reduced pressure. The obtained crude material was purified by silica gel column chromatography eluting with 8% MeOH in DCM to afford I-8 as an off-white solid (40 mg, 0.08 mmol, 21% yield). MS [M+H]⁺=463.1. ¹H NMR (400 MHz, DMSO-d₆): δ 10.97 (s, 1H), 8.03 (d, J=2.0 Hz, 1H), 7.65-7.62 (m, 2H), 7.48 (s, 1H), 7.39 (d, J=8.0 Hz, 1H), 6.76 (d, J=8.4 Hz, 1H), 5.09 (dd J=13.2, 4.8 Hz, 1H), 4.41 (d, J=17.2 Hz, 1H), 4.30-4.24 (m, 3H), 3.44 (s, 2H), 2.94-2.87 (m, 3H), 2.67-2.32 (m, 3H), 2.07-1.95 (m, 3H), 1.77-1.65 (m, 4H), 1.30 (t, J=7.2 Hz, 3H).

### Example 23: 3-(5-(1-(2-(1H-pyrazol-1-yl)ethyl)piperidin-4-yl)-1-oxoisoindolin-2-yl)piperidine-2,6-dione (I-11)

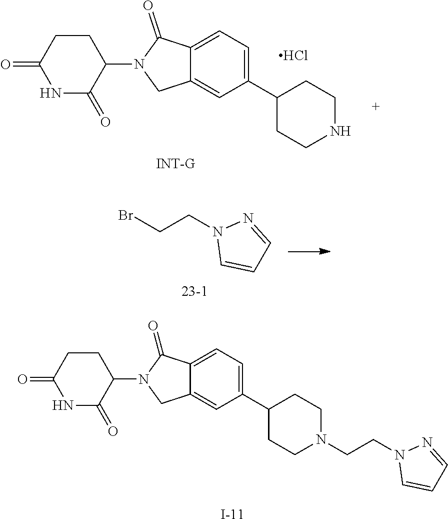

To a stirred solution of INT-G (150 mg, 0.41 mmol) and Et3N (0.17 mL, 1.23 mmol) in DMF (10 mL) was added 23-1 (142 mg, 0.82 mmol) at 0° C. and the resulting mixture was stirred at rt for 16 h. Upon complete consumption of the starting materials, the reaction mixture was quenched with ice-cold water and extracted with EtOAc (2×75 mL). The combined organic extracts were washed with brine, dried over Na₂SO₄, filtered, and concentrated under reduced pressure. The obtained crude material was purified by silica gel column chromatography eluting with 6% MeOH in DCM to afford I-11 as an off-white solid (110 mg, 0.26 mmol, 63% yield). MS [M+H]⁺=422.3. ¹H NMR (400 MHz, DMSO-d₆): δ 10.99 (s, 1H), 7.75 (d, J=2.0 Hz, 1H), 7.63 (d, J=8.0 Hz, 1H), 7.48 (s, 1H), 7.42-7.38 (m, 2H), 6.22-6.21 (m, 1H), 5.09 (dd, J=13.2, 4.8 Hz, 1H), 4.42 (d, J=17.2 Hz, 1H), 4.30-4.22 (m, 3H), 2.99-2.86 (m, 3H), 2.75-2.55 (m, 3H), 2.42-2.31 (m, 2H), 2.15-1.92 (m, 3H), 1.78-1.62 (m, 4H).

### Example 24: 4-((4-(2-(2,6-dioxopiperidin-3-yl)-1-oxoisoindolin-5-yl)piperidin-1-yl)methyl)cyclohexane-1-carbonitrile (I-3)

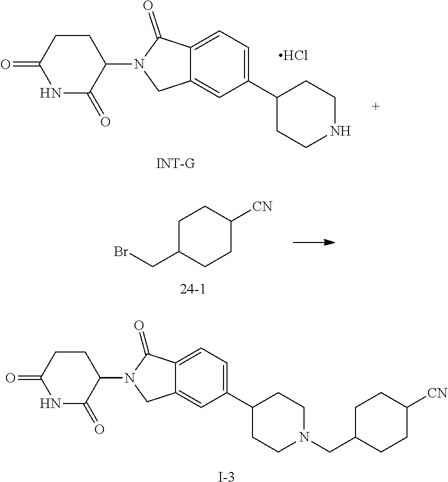

To a stirred solution of INT-G (150 mg, 0.41 mmol) and Et3N (0.17 mL, 1.23 mmol) in DMF (10 mL) was added 24-1 (99 mg, 0.49 mmol) at 0° C. and the resulting mixture was stirred at rt for 16 h. Upon complete consumption of the starting materials, the reaction mixture was quenched with ice-cold water and extracted with EtOAc (2×75 mL). The combined organic extracts were washed with brine, dried over Na₂SO₄, filtered, and concentrated under reduced pressure. The obtained crude material was purified by silica gel column chromatography eluting with 6% MeOH in DCM to afford I-3 as an off-white solid (30 mg, 0.07 mmol, 16% yield). MS [M+H]⁺=449.2. ¹H NMR (400 MHz, DMSO-d₆): δ 10.99 (s, 1H), 7.63 (d, J=8.0 Hz, 1H), 7.49 (s, 1H), 7.39 (d, J=8.0 Hz, 1H), 5.09 (dd, J=13.2, 4.8 Hz, 1H), 4.41 (d, J=17.2 Hz, 1H), 4.27 (d, J=17.2 Hz, 1H), 3.34-3.28 (m, 2H), 3.29-2.87 (m, 3H), 2.67-2.32 (m, 3H), 2.10-2.09 (m, 2H), 2.08-1.93 (m, 5H), 1.80-1.66 (m, 5H), 1.53-1.45 (m, 3H), 0.90-0.86 (m, 2H).

### Example 25: 3-(1-oxo-5-(1-(2-(pyridin-4-yl)propan-2-yl)piperidin-4-yl)isoindolin-2-yl)piperidine-2,6-dione HCOOH salt (I-37)

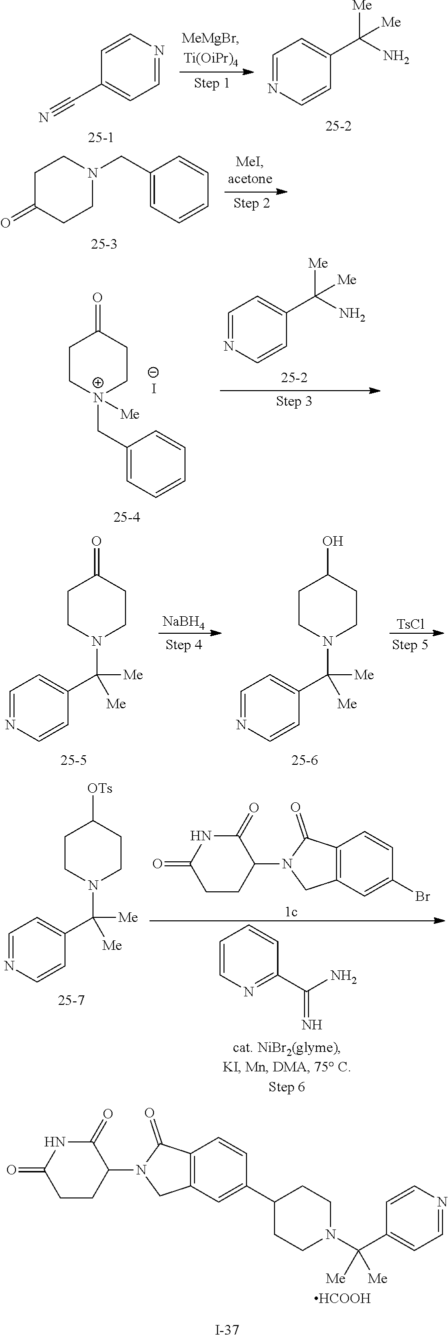

### Step 1: 2-(pyridin-4-yl)propan-2-amine (25-2)

To a solution of compound 25-1 (5.00 g, 48.0 mmol) in Et20 (100 mL) was added MeMgBr (3M in THF, 48.0 mL, 144 mmol) at 0° C. and the reaction mixture was stirred at room temperature for 30 minutes. To the reaction mixture was added Ti(OiPr)₄ (14.2 mL, 48.0 mmol) and stirred at 50° C. for 16 h. After completion of the reaction, the reaction mixture was quenched with 1M aq. NaOH and extracted with Et20 (3×100 mL). The combined organic extracts were washed with brine, dried over Na₂SO₄ and concentrated under reduced pressure. The obtained crude material was purified by silica gel column chromatography eluting with 5-10% MeOH in DCM to afford compound 25-2 (1.80 g, 13.2 mmol, 28%) as a viscous yellow liquid. ¹H NMR (400 MHz, DMSO-d₆): δ 8.46-8.45 (m, 2H), 7.50-7.49 (m, 2H), 1.96 (brs, 2H), 1.33 (s, 6H)

### Step 2: 1-benzyl-1-methyl-4-oxopiperidin-1-ium (25-4)

To a solution of compound 25-3 (5.0 g, 26 mmol) in acetone (100 mL), Mel (2.0 mL, 32 mmol) was added. The reaction mixture was stirred at room temperature for 16 h. After completion of the reaction, the reaction mixture was filtered, washed with acetone and dried reduced pressure to afford compound 25-4 (4.0 g, 12 mmol, 46%) as a white solid. ¹H NMR (300 MHz, DMSO-d₆): δ 7.56-7.55 (m, 5H), 4.73 (s, 2H), 3.85-3.62 (m, 4H), 3.14 (s, 3H), 2.89-2.75 (m, 2H), 2.60-2.72 (m, 2H).

### Step 3: 1-(2-(pyridin-4-yl)propan-2-yl)piperidin-4-one (25-5)

To a solution of compound 25-2 (2.0 g, 6.0 mmol) in EtOH:H₂O (v/v=3:2) (30 mL) was added compound 25-4 (1.20 g, 9.05 mmol) and K₂CO₃ (125 mg, 0.90 mmol). The reaction mixture was stirred at 100° C. for 12 h. After completion of the reaction, the reaction mixture was diluted with water and extracted with EtOAc (3×50 mL). The combined organic extracts were washed with brine, dried over Na₂SO₄ and concentrated under reduced pressure. The obtained crude material was purified by silica gel column chromatography eluting with 50% EtOAc in Hexane to afford compound 25-5 (0.60 g, 2.7 mmol, 46%) as brown liquid. ¹H NMR (600 MHz, CDCl₃): δ 8.56-8.55 (d, J=6.0 Hz, 2H), 7.50-7.49 (d, J=4.8 Hz, 2H), 2.75-2.73 (t, J=6.0 Hz, 4H), 2.42-2.40 (t, J=6.0 Hz, 4H), 1.38 (s, 6H).

### Step 4: 1-(2-(pyridin-4-yl)propan-2-yl)piperidin-4-ol (25-6)

To a solution of 25-5 (232 mg, 1.06 mmol) in MeOH (3 mL) was added NaBH₄ (48 mg, 1.3 mmol) portion wise at 0° C. The reaction mixture was stirred for 30 min at rt. The reaction mixture was quenched with sat. aq. NaHCO₃ and extracted with DCM (×3). Combined organic phases were dried over Na₂SO₄, filtered and concentrated to dryness to afford compound 25-6 (233 mg, 1.06 mmol, 100% yield) as a colorless oil. The product was used in the next step without further purification. MS [M+H]⁺=221.4. ¹H NMR (400 MHz, Chloroform-d) δ 8.54 (d, J=5.3 Hz, 2H), 7.47 (d, J=5.2 Hz, 2H), 3.79-3.57 (m, 1H), 2.77-2.63 (m, 2H), 2.25 (t, J=10.2 Hz, 2H), 1.94-1.81 (m, 2H), 1.61-1.51 (m, 2H), 1.34 (s, 6H).

### Step 5: 1-(2-(pyridin-4-yl)propan-2-yl)piperidin-4-yl 4-methylbenzenesulfonate (25-7)

To a solution of 25-6 (233 mg, 1.06 mmol), DIPEA (0.28 mL, 1.6 mmol), DMAP (13 mg, 0.11 mmol) in DCM (10 mL) was added TsCl (121 mg, 1.06 mmol) and reaction mixture was stirred for 1 h at 0° C. and then overnight at rt. The reaction mixture was quenched with sat. aq. NaHCO₃ and extracted with DCM (×3). The combined organic phases were dried over Na₂SO₄, filtered, and concentrated to dryness to afford crude 25-7 (390 mg, assumed quantitative yield) as a brown oil. The obtained crude product was used in the next step without further purification.

### Step 6: 3-(1-oxo-5-(1-(2-(pyridin-4-yl)propan-2-yl)piperidin-4-yl)isoindolin-2-yl)piperidine-2,6-dione HCOOH salt (I-37)

To a suspension of NiBr₂(glyme) (7.2 mg, 0.023 mmol), picolinimidamide HCl salt (3.7 mg, 0.023 mmol), KI (77 mg, 0.46 mmol) and manganese powder (43 mg, 0.77 mmol) in DMA (0.2 mL) was added a mixture of 1c (50 mg, 0.16 mmol) and crude 25-7 (174 mg, 0.465 mmol), dissolved in DMA (1 mL) under nitrogen atmosphere. The reaction mixture was then stirred vigorously at 75° C. for 7 hours under atmosphere of nitrogen. The reaction was filtered and the filter was washed with MeCN. The reaction mixture was then concentrated. The crude product was purified by RP HPLC (eluting with MeCN/H₂O with 0.10% NH₃ as a modifier) using collecting into tubes containing ˜2 drops of HCOOH. Fractions containing desired product were concentrated by lyophilizer to afford the formate salt of compound I-37 (6.9 mg, 0.014 mmol, 9% yield) was obtained as a white solid. MS [M+H]⁺=447.5. ¹H NMR (400 MHz, CD₃CN:D₂O (v/v=1:1)) δ 8.61 (d, J=5.9 Hz, 2H), 8.33 (s, 1H), 7.74-7.68 (m, 1H), 7.64 (d, J=5.3 Hz, 2H), 7.43 (d, J=3.8 Hz, 1H), 7.38 (d, J=8.3 Hz, 1H), 5.12-4.94 (m, 1H), 4.50-4.27 (m, 2H), 3.32 (d, J=11.7 Hz, 2H), 2.93-2.69 (m, 5H), 2.47-2.30 (m, 1H), 2.18-2.08 (m, 1H), 1.72 (s, 6H). Missing protons are overlapping with residual acetonitrile or H₂O solvent peaks.

### Biological Assays and Data

The activity of a compound according to the present disclosure can be assessed by the following in vitro methods.

### Example 26: Prolabel Quantification of IKZF1 or IKZF2 Protein Levels in 293GT Cells

The Prolabel system from DiscoverX was used to develop high-throughput and quantitative assays to measure changes in IKZF1 and IKZF2 protein levels in response to compounds. The prolabel tag was derived from the alpha fragment of beta galactosidase and has the following protein sequence: mssnslavvlqrrdwenpgvtglnrlaahppfaswrnseeartdrpsqqlrsinge. The complementary fragment of beta-galactosidase (from DiscoverX), is added to the prolabel tag to form an active beta galactosidase enzyme whose activity can be precisely measured. In this way, the levels of a fusion protein with the prolabel tag can be quantified in cell lysates.

Lentiviral vectors, based on the Invitrogen pLenti6.2/V5 DEST backbone, were constructed that placed the prolabel tag upstream of IKZF1, IKZF2 or GSPT1 and expressed the fusion protein from a CMV promoter.

To ensure moderate and consistent expression of the prolabel fusion proteins across all cells in the population, stable cell lines were constructed from cells expressing a single copy of the construct. Lentivirus packaged with the constructs was made using the Virapower kit from Invitrogen. Strongly adherent 293GT cell, GripTite 293 MSR cells from Thermo Fisher Scientific (Catalog number: R79507), were infected with the virus at low multiplicity of infection and selected by 5 μg/mL blasticidin for 2 weeks.

The levels of prolabel tagged fusion proteins in compound treated cell lines were measured as follows: Day 1, Cells were diluted to 1.0×10⁶ cells/ml in normal growth medium. 17.5 μL of cells were plated in each well of a solid white 384 well plate. Plates were incubated overnight in a 37° C. tissue culture incubator.

Day 2, Serial dilutions of compounds were made in 384 well plates from 10 mM stocks. 15 μL of DMSO was added to each well of a 384 well plate. In the first column, 15 μL of stock compound was added. The solution was mixed and 15 μL was transferred to the next column. This was repeated until 20 two-fold dilutions were prepared. 2.5 μL of diluted compounds were transferred into 60 μL of cell culture medium in another 384 well plate, and mixed well. 2.5 μL of this mixture was added to the plated cells. The final DMSO concentration was 0.5% and the highest concentration of compound was 50 μM. Plates were incubated overnight (e.g., about 14 h, 18 h, or 24 h) in a 37° C. tissue culture incubator.

Day 3, Plates were removed from the incubator and allowed to equilibrate at rt for 30 minutes. Prolabel substrate (DiscoverX PathHunter Prolabel Detection Kit, User manual: 93-0180) was added as described by the manufacturers protocols. Plates were incubated at rt for three hours and luminescence was read using an Envision reader (Perkin Elmer) Data was analyzed and visualized using the Spotfire software package.

Table 14 shows Helios (IKZF2), Ikaros (IKZF1) and G1 to S phase transition 1 protein (GSPT1) degradation activity of compounds of the disclosure in Pro-label assays in 293GT cells, (% degradation is at 10 μM). Pomalidomide was tested as the control.

TABLE 14
IKZF2 and IKZF1 Activity

| | IKZF2 | | |
|---|---|---|---|
| | | % protein | |
| | | reduction | IKZF1 |
| Cmpd | AC₅₀ | at 10 μM, | AC₅₀ |
| No. | (μM) | 24 h | (μM) |
| I-1 | 2.2 | 70% | >30 |
| I-2 | — | 35% at | >30 |
| | | 30 uM | |
| I-3 | 0.089 | 60% | >30 |
| I-4 | — | 30% at | >30 |
| | | 30 uM | |
| I-5 | 0.025 | 60% | >30 |
| I-6 | 0.009 | 50% | >30 |
| I-7 | 0.024 | 80% | >30 |
| I-8 | 0.014 | 70% | >30 |
| I-10 | 0.017 | 75% | >30 |
| I-11 | 0.14 | 60% | >30 |
| I-12 | — | — | >30 |
| I-13 | 0.012 | 80% | >30 |
| I-14 | 2.86 | 55% | >30 |
| I-15 | 1.87 | 40% | >30 |
| I-16 | 13.1 | 50% | >30 |
| I-17 | 1.73 | 50% | >30 |
| I-18 | 10.0 | 40% | >30 |
| I-30 | 0.020 | 80% | >30 |
| I-31 | 0.079 | 70% | >30 |
| I-32 | 0.092 | 62% | >30 |
| I-33 | 0.064 | 62% | >30 |
| I-35 | 0.019 | 80% | >30 |
| I-36 | 0.038 | 74% | >30 |
| Control | >50 | | 0.05 |
| | | | (80% |
| | | | degradation |
| | | | at 10 μM) |

### Example 26: Quantification of In Vitro Suppressive Potency of Primary Human Regulatory T Cells Expanded in the Presence of Compounds

### Materials and Methods

### Treg Cell Sorting

Human buffy coats are obtained from BioreclamationIVT, in the USA. CD4+ T cells are isolated from said buffy coats using the RosetteSep Human CD4+ T cell enrichment Cocktail (Stemcell technologies, USA) and gradient centrifugation over Ficoll Paque Plus (GE HealthCare LifeSciences, USA) as per manufacturer's recommendations. Cells are resuspended in RPMI medium supplemented with 1% penicillin-Streptomycin solution, 10% Fetal Bovine Serum, HEPES (10 mM), MEM NEAA (100 nM), sodium pyruvate (1 mM) (all supplements from Thermo Fisher Scientific, USA), thereafter referred to as complete RPMI (cRPMI), and rested overnight at 37° C., 5% CO₂ in the presence of 2 U/mL rhIL-2 (Proleukin, Novartis). Cells are collected and resuspended in autoMACS Running Buffer supplemented with BSA (Miltenyi Biotec, USA) and labelled using CD4-FITC antibody (clone RPA-T4), CD25-APC antibody (clone M-A251) (Biolegend) and CD25 Microbeads (Miltenyi Biotec, USA). CD25-enriched cells are then isolated using the autoMACS Pro Separator. A highly purified population of Treg cells is then obtained by further sorting CD4+CD25Hi cells using a Sony SH800 cell sorter. The resulting Treg cell population is routinely above 90% pure according to FOXP3 expression.

### Treg Cell Expansion:

Purified Treg cells are plated in cRPMI in 96-well, round-bottom plates at a density of 25000-50000 cells per well and activated in the presence of 500 U/mL rhIL2, and Treg expander Dynabeads (Thermo Fisher Scientific, USA) according to manufacturer's recommendations, in the presence or absence of 100 μM rapamycin (Thermo Fisher Scientific, USA). The compounds of the present disclosure are then added at a final concentration of 10 M and DMSO was added as a vehicle control. Cells are incubated at 37° C., 5% CO₂ for a total of 12-14 days. The compound and rhIL2 are replenished every 48 h during the entirety of the culture.

### Phenotypic Analysis of Expanded Treg Cells:

Cell are collected and counted and the fold expansion is calculated as (number of cells recovered)/(number of cells plated). A fraction of the cells is fixed and permeabilized using the eBioscience Foxp3 staining Buffer kit (eBioscience, Thermo Fisher Scientific, USA) and stained with Helios-PECyanine7 antibody (Clone 22F6). To determine IL2-expression, expanded Treg cells are further incubated in the presence of the eBioscience Cell Stimulation Cocktail with Protein inhibitors (Thermo Fisher Scientific) for 4 hours, followed by fixation and staining with IL2-BV711 antibody (clone MQ1-17H12) (Biolegend, USA). Cells are acquired on an LSRFortessa (Becton Dickinson, USA) and analysis was performed using the FlowJo software (TreeStar, USA).

### Functional Analysis of Expanded Treg Cells:

Primary human PBMCs are obtained from freshly prepared buffy coats (BioReclamationIVT) using gradient centrifugation over Ficoll Paque Plus as per manufacturer's recommendations. Cells are then labelled with CFSE (5(6)-Carboxyfluorescein diacetate N-succinimidyl ester, Sigma-Aldrich, USA) and plated in triplicates cRPMI in round bottom 96-well plates, alone or with expanded Treg cells at a 1:2 PBMC:Treg ratio. The compounds of the present disclosure are then added at a final concentration of 10 M and DMSO is added as a vehicle control. Cells are activated using soluble anti-CD3 antibody (clone OKT3) (eBioscience, ThermoFisher Scientific, USA) at a final concentration of 100 ng/ml. Cells are incubated at 37° C., 5% CO₂ for a total of 4-5 days. At the end of the culture, cells are stained using the Live/dead Blue viability stain (Thermo Fisher Scientific, USA) as per manufacturer's instructions, followed by staining with CD4-BUV737 (Clone SK3) (BDBiosciences, USA) and CD8-BV711 (clone RPA-T8) (Biolegend, USA). Cells are acquired on an LSRFortessa (Becton Dickinson, USA) and analysis is performed using the FlowJo software (TreeStar, USA). Proliferation is assessed in each population as the proportion of cells having diluted CFSE. Suppression is assessed for each condition in comparison to the responders plated alone.

Those skilled in the art will recognize, or be able to ascertain, using no more than routine experimentation, numerous equivalents to the specific embodiments described specifically herein. Such equivalents are intended to be encompassed in the scope of the following claims.

What is claimed is:

1. A compound selected from:
3-(5-(1-benzyl-4-hydroxypiperidin-4-yl)-1-oxoisoindolin-2-yl)piperidine-2,6-dione;
3-(5-(1-benzyl-4-methoxypiperidin-4-yl)-1-oxoisoindolin-2-yl)piperidine-2,6-dione;
3-(5-(1-benzyl-4-fluoropiperidin-4-yl)-1-oxoisoindolin-2-yl)piperidine-2,6-dione;
1-benzyl-4-(2-(2,6-dioxopiperidin-3-yl)-1-oxoisoindolin-5-yl)piperidine-4-carbonitrile;
3-(5-(4-amino-1-benzylpiperidin-4-yl)-1-oxoisoindolin-2-yl)piperidine-2,6-dione;
3-(5-(3-benzyl-3-azabicyclo[4.1.0]heptan-6-yl)-1-oxoisoindolin-2-yl)piperidine-2,6-dione;
3-(5-(3-(((1r,4r)-4-methoxycyclohexyl)methyl)-3-azabicyclo[4.1.0]heptan-6-yl)-1-oxoisoindolin-2-yl)piperidine-2,6-dione;
3-(5-(4-fluoro-1-(((1r,4r)-4-methoxycyclohexyl)methyl)piperidin-4-yl)-1-oxoisoindolin-2-yl)piperidine-2,6-dione;
3-(5-(4-hydroxy-1-(((1r,4r)-4-methoxycyclohexyl)methyl)piperidin-4-yl)-1-oxoisoindolin-2-yl)piperidine-2,6-dione;
3-(5-(4-methoxy-1-(((1r,4r)-4-methoxycyclohexyl)methyl)piperidin-4-yl)-1-oxoisoindolin-2-yl)piperidine-2,6-dione;
3-(5-(4-amino-1-(((1r,4r)-4-methoxycyclohexyl)methyl)piperidin-4-yl)-1-oxoisoindolin-2-yl)piperidine-2,6-dione; and
4-(2-(2,6-dioxopiperidin-3-yl)-1-oxoisoindolin-5-yl)-1-(((1r,4r)-4-methoxycyclohexyl)methyl)piperidine-4-carbonitrile;
or a pharmaceutically acceptable salt, thereof.

2. A compound selected from:
3-(1-oxo-5-(1-((6-oxo-1,6-dihydropyridin-3-yl)methyl)piperidin-4-yl)isoindolin-2-yl)piperidine-2,6-dione;
3-(5-(1-benzyl-2,6-dimethylpiperidin-4-yl)-1-oxoisoindolin-2-yl)piperidine-2,6-dione;
(1r,4r)-4-((4-(2-(2,6-dioxopiperidin-3-yl)-1-oxoisoindolin-5-yl)piperidin-1-yl)methyl)cyclohexane-1-carbonitrile;
3-(5-(2,6-dimethylpiperidin-4-yl)-1-oxoisoindolin-2-yl)piperidine-2,6-dione;
3-(5-(1-((5-ethoxypyridin-2-yl)methyl)piperidin-4-yl)-1-oxoisoindolin-2-yl)piperidine-2,6-dione;
3-(5-(1-((5-methoxypyridin-2-yl)methyl)piperidin-4-yl)-1-oxoisoindolin-2-yl)piperidine-2,6-dione;
3-(5-(1-((6-methoxypyridin-3-yl)methyl)piperidin-4-yl)-1-oxoisoindolin-2-yl)piperidine-2,6-dione;
3-(5-(1-((6-ethoxypyridin-3-yl)methyl)piperidin-4-yl)-1-oxoisoindolin-2-yl)piperidine-2,6-dione;
3-(5-(1-((5-methyl-1H-imidazol-4-yl)methyl)piperidin-4-yl)-1-oxoisoindolin-2-yl)piperidine-2,6-dione;
3-(5-(1-((4-(fluoromethyl)cyclohexyl)methyl)piperidin-4-yl)-1-oxoisoindolin-2-yl)piperidine-2,6-dione;
3-(5-(1-(2-(1H-pyrazol-1-yl)ethyl)piperidin-4-yl)-1-oxoisoindolin-2-yl)piperidine-2,6-dione
3-(5-(1-((4-methylpyrimidin-5-yl)methyl)piperidin-4-yl)-1-oxoisoindolin-2-yl)piperidine-2,6-dione;
3-(5-(1-((4-methylcyclohex-3-en-1-yl)methyl)piperidin-4-yl)-1-oxoisoindolin-2-yl)piperidine-2,6-dione;
3-(1-oxo-5-(1-(pyrazin-2-ylmethyl)piperidin-4-yl)isoindolin-2-yl)piperidine-2,6-dione;
3-(1-oxo-5-(1-(pyridazin-3-ylmethyl)piperidin-4-yl)isoindolin-2-yl)piperidine-2,6-dione;
3-(1-oxo-5-(1-(pyrimidin-4-ylmethyl)piperidin-4-yl)isoindolin-2-yl)piperidine-2,6-dione;
3-(5-(1-((2-methylpyrimidin-5-yl)methyl)piperidin-4-yl)-1-oxoisoindolin-2-yl)piperidine-2,6-dione;
3-(1-oxo-5-(1-(pyridazin-4-ylmethyl)piperidin-4-yl)isoindolin-2-yl)piperidine-2,6-dione;
3-(5-((1R,4S)-2-benzyl-2-azabicyclo[2.2.2]octan-5-yl)-1-oxoisoindolin-2-yl)piperidine-2,6-dione;
3-(5-((1R,5S)-9-benzyl-3-methyl-3,9-diazabicyclo[3.3.1]nonan-7-yl)-1-oxoisoindolin-2-yl)piperidine-2,6-dione;
3-(5-((1R,5S)-9-benzyl-3-oxa-9-azabicyclo[3.3.1]nonan-7-yl)-1-oxoisoindolin-2-yl)piperidine-2,6-dione;
3-(5-((1R,5S)-9-benzyl-3,9-diazabicyclo[3.3.1]nonan-7-yl)-1-oxoisoindolin-2-yl)piperidine-2,6-dione;
3-(5-((1S,4S)-2-benzyl-2-azabicyclo[2.2.1]heptan-5-yl)-1-oxoisoindolin-2-yl)piperidine-2,6-dione;
3-(5-((1R,5S)-9-ethyl-3-methyl-3,9-diazabicyclo[3.3.1]nonan-7-yl)-1-oxoisoindolin-2-yl)piperidine-2,6-dione;
3-(1-oxo-5-(1-(1-(tetrahydro-2H-pyran-4-yl)ethyl)piperidin-4-yl)isoindolin-2-yl)piperidine-2,6-dione;
3-(5-(1-(1-(1-ethyl-1H-pyrazol-4-yl)ethyl)piperidin-4-yl)-1-oxoisoindolin-2-yl)piperidine-2,6-dione;
3-(5-(1-(1-(1-ethyl-1H-pyrazol-4-yl)propyl)piperidin-4-yl)-1-oxoisoindolin-2-yl)piperidine-2,6-dione;
3-(1-oxo-5-(1-(1-(pyrazin-2-yl)propyl)piperidin-4-yl)isoindolin-2-yl)piperidine-2,6-dione;
3-(1-oxo-5-(1-(1-(pyridazin-4-yl)propyl)piperidin-4-yl)isoindolin-2-yl)piperidine-2,6-dione;
3-(5-(1-(6,7-dihydro-5H-cyclopenta[b]pyridin-5-yl)piperidin-4-yl)-1-oxoisoindolin-2-yl)piperidine-2,6-dione;
3-(5-(1-(4-methoxycyclohexyl)piperidin-4-yl)-1-oxoisoindolin-2-yl)piperidine-2,6-dione;
3-(1-oxo-5-(1-(2-(pyridin-4-yl)propan-2-yl)piperidin-4-yl)isoindolin-2-yl)piperidine-2,6-dione; and
3-(1-oxo-5-(1-((2-oxo-1,2-dihydropyridin-3-yl)methyl)piperidin-4-yl)isoindolin-2-yl)piperidine-2,6-dione;
or a pharmaceutically acceptable salt, thereof.

3. A pharmaceutical composition comprising a therapeutically effective amount of a compound according to claim 1 or claim 2, or a pharmaceutically acceptable salt, thereof, and a pharmaceutically acceptable carrier or excipient.

4. The pharmaceutical composition according to claim 3 further comprising at least one additional pharmaceutical agent.

5. The pharmaceutical composition according to claim 3 or claim 4 for use in the treatment of a disease or disorder that is affected by the reduction of IKZF2 protein levels.

6. A method of degrading IKZF2 comprising administering to the patient in need thereof a compound according to claim 1 or claim 2, or a pharmaceutically acceptable salt, thereof.

7. A method of treating a disease or disorder that is affected by the modulation of IKZF2 protein levels comprising administering to the patient in need thereof a compound according to claim 1 or claim 2, or a pharmaceutically acceptable salt thereof.

8. A method of modulating IKZF2 protein levels comprising administering to the patient in need thereof a compound according to claim 1 or claim 2, or a pharmaceutically acceptable salt, thereof.

9. A method of reducing the proliferation of a cell the method comprising, contacting the cell with a compound according to claim 1 or claim 2, or a pharmaceutically acceptable salt thereof, and reducing IKZF2 protein levels.

10. A method of treating cancer comprising administering to the patient in need thereof a compound according to claim 1 or claim 2, or a pharmaceutically acceptable salt, thereof.

11. The method according to claim 10, wherein the cancer is selected from non-small cell lung cancer (NSCLC), melanoma, triple-negative breast cancer (TNBC), nasopharyngeal cancer (NPC), microsatellite stable colorectal cancer (mssCRC), thymoma, carcinoid, acute myelogenous leukemia, and gastrointestinal stromal tumor (GIST).

12. The method according to claim 10, wherein the cancer is a cancer for which the immune response is deficient or an immunogenic cancer.

13. A method for reducing IKZF2 protein levels in a subject comprising the step of administering to a subject in need thereof a therapeutically effective amount of a compound according to claim 1 or claim 2, or a pharmaceutically acceptable salt thereof.

14. A compound according to claim 1 or claim 2, or a pharmaceutically acceptable salt, thereof, for use in the treatment of a disease or disorder that is affected by the reduction of IKZF2 protein levels.


SEQUENCE LISTING (314 sequences)

SEQ ID NO: 1 - protein, 121 aa, synthetic construct
  REGION 1..121: note = source = /note="Description of Artificial Sequence: Synthetic polypeptide"
EVQLVESGGGLVQSGGSLRLSCAASGFSLSSYGVDWVRQAPGKGLEWVGVIWGGGGTYYASSLMGRFTISRDNSKNTLYLQMNSLRAEDTAVYYCARHAYGHDGGFAMDYWGQGTLVTVSS

SEQ ID NO: 2 - protein, 107 aa, synthetic construct
  REGION 1..107: note = source = /note="Description of Artificial Sequence: Synthetic polypeptide"
EIVMTQSPATLSVSPGERATLSCRASESVSSNVAWYQQRPGQAPRLLIYGASNRATGIPARFSGSGSGTDFTLTISRLEPEDFAVYYCGQSYSYPFTFGQGTKLEIK

SEQ ID NO: 3 - protein, 451 aa, synthetic construct
  REGION 1..451: note = source = /note="Description of Artificial Sequence: Synthetic polypeptide"
EVQLVESGGGLVQSGGSLRLSCAASGFSLSSYGVDWVRQAPGKGLEWVGVIWGGGGTYYASSLMGRFTISRDNSKNTLYLQMNSLRAEDTAVYYCARHAYGHDGGFAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 214 aa, synthetic construct
  REGION 1..214: note = source = /note="Description of Artificial Sequence: Synthetic polypeptide"
EIVMTQSPATLSVSPGERATLSCRASESVSSNVAWYQQRPGQAPRLLIYGASNRATGIPARFSGSGSGTDFTLTISRLEPEDFAVYYCGQSYSYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - DNA, 363 bp, synthetic construct
  misc_feature 1..363: note = source = /note="Description of Artificial Sequence: Synthetic polynucleotide"
  source 1..363: mol_type = other DNA
gaggtgcagctggtggaatctggcggcggactggtgcagtccggcggctctctgagactgtcttgcgctgcctccggcttctccctgtcctcttacggcgtggactgggtgcgacaggcccctggcaagggcctggaatgggtgggagtgatctggggcggaggcggcacctactacgcctcttccctgatgggccggttcaccatctcccgggacaactccaagaacaccctgtacctgcagatgaactccctgcgggccgaggacaccgccgtgtactactgcgccagacacgcctacggccacgacggcggcttcgccatggattattggggccagggcaccctggtgacagtgtcctcc

SEQ ID NO: 6 - DNA, 321 bp, synthetic construct
  misc_feature 1..321: note = source = /note="Description of Artificial Sequence: Synthetic polynucleotide"
  source 1..321: mol_type = other DNA
gagatcgtgatgacccagtcccccgccaccctgtctgtgtctcccggcgagagagccaccctgagctgcagagcctccgagtccgtgtcctccaacgtggcctggtatcagcagagacctggtcaggcccctcggctgctgatctacggcgcctctaaccgggccaccggcatccctgccagattctccggctccggcagcggcaccgacttcaccctgaccatctcccggctggaacccgaggacttcgccgtgtactactgcggccagtcctactcataccccttcaccttcggccagggcaccaagctggaaatcaag

SEQ ID NO: 7 - DNA, 1353 bp, synthetic construct
  misc_feature 1..1353: note = source = /note="Description of Artificial Sequence: Synthetic polynucleotide"
  source 1..1353: mol_type = other DNA
gaggtgcagctggtggaatctggcggcggactggtgcagtccggcggctctctgagactgtcttgcgctgcctccggcttctccctgtcctcttacggcgtggactgggtgcgacaggcccctggcaagggcctggaatgggtgggagtgatctggggcggaggcggcacctactacgcctcttccctgatgggccggttcaccatctcccgggacaactccaagaacaccctgtacctgcagatgaactccctgcgggccgaggacaccgccgtgtactactgcgccagacacgcctacggccacgacggcggcttcgccatggattattggggccagggcaccctggtgacagtgtcctccgctagcaccaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 8 - DNA, 642 bp, synthetic construct
  misc_feature 1..642: note = source = /note="Description of Artificial Sequence: Synthetic polynucleotide"
  source 1..642: mol_type = other DNA
gagatcgtgatgacccagtcccccgccaccctgtctgtgtctcccggcgagagagccaccctgagctgcagagcctccgagtccgtgtcctccaacgtggcctggtatcagcagagacctggtcaggcccctcggctgctgatctacggcgcctctaaccgggccaccggcatccctgccagattctccggctccggcagcggcaccgacttcaccctgaccatctcccggctggaacccgaggacttcgccgtgtactactgcggccagtcctactcataccccttcaccttcggccagggcaccaagctggaaatcaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 9 - protein, 5 aa, synthetic construct
  REGION 1..5: note = source = /note="Description of Artificial Sequence: Synthetic peptide"
SYGVD

SEQ ID NO: 10 - protein, 7 aa, synthetic construct
  REGION 1..7: note = source = /note="Description of Artificial Sequence: Synthetic peptide"
GFSLSSY

SEQ ID NO: 11 - protein, 16 aa, synthetic construct
  REGION 1..16: note = source = /note="Description of Artificial Sequence: Synthetic peptide"
VIWGGGGTYYASSLMG

SEQ ID NO: 12 - protein, 5 aa, synthetic construct
  REGION 1..5: note = source = /note="Description of Artificial Sequence: Synthetic peptide"
WGGGG

SEQ ID NO: 13 - protein, 13 aa, synthetic construct
  REGION 1..13: note = source = /note="Description of Artificial Sequence: Synthetic peptide"
HAYGHDGGFAMDY

SEQ ID NO: 14 - protein, 11 aa, synthetic construct
  REGION 1..11: note = source = /note="Description of Artificial Sequence: Synthetic peptide"
RASESVSSNVA

SEQ ID NO: 15 - protein, 7 aa, synthetic construct
  REGION 1..7: note = source = /note="Description of Artificial Sequence: Synthetic peptide"
SESVSSN

SEQ ID NO: 16 - protein, 7 aa, synthetic construct
  REGION 1..7: note = source = /note="Description of Artificial Sequence: Synthetic peptide"
GASNRAT

SEQ ID NO: 17 - skipped

SEQ ID NO: 18 - protein, 9 aa, synthetic construct
  REGION 1..9: note = source = /note="Description of Artificial Sequence: Synthetic peptide"
GQSYSYPFT

SEQ ID NO: 19 - protein, 6 aa, synthetic construct
  REGION 1..6: note = source = /note="Description of Artificial Sequence: Synthetic peptide"
SYSYPF

SEQ ID NO: 20 - protein, 124 aa, synthetic construct
  REGION 1..124: note = source = /note="Description of Artificial Sequence: Synthetic polypeptide"
QVQLVESGGGVVQPGRSLRLSCAASGFTFSSYGMHWVRQAPGKGLEWVAVIWYEGSNKYYADSVKGRFTISRDNSKNTLYLQMNSLRAEDTAVYYCARGGSMVRGDYYYGMDVWGQGTTVTVSS

SEQ ID NO: 21 - protein, 107 aa, synthetic construct
  REGION 1..107: note = source = /note="Description of Artificial Sequence: Synthetic polypeptide"
AIQLTQSPSSLSASVGDRVTITCRASQGISSALAWYQQKPGKAPKLLIYDASSLESGVPSRFSGSGSGTDFTLTISSLQPEDFATYYCQQFNSYPYTFGQGTKLEIK

SEQ ID NO: 22 - protein, 5 aa, synthetic construct
  REGION 1..5: note = source = /note="Description of Artificial Sequence: Synthetic peptide"
TYWMH

SEQ ID NO: 23 - protein, 17 aa, synthetic construct
  REGION 1..17: note = source = /note="Description of Artificial Sequence: Synthetic peptide"
NIYPGTGGSNFDEKFKN

SEQ ID NO: 24 - protein, 8 aa, synthetic construct
  REGION 1..8: note = source = /note="Description of Artificial Sequence: Synthetic peptide"
WTTGTGAY

SEQ ID NO: 25 - protein, 7 aa, synthetic construct
  REGION 1..7: note = source = /note="Description of Artificial Sequence: Synthetic peptide"
GYTFTTY

SEQ ID NO: 26 - protein, 6 aa, synthetic construct
  REGION 1..6: note = source = /note="Description of Artificial Sequence: Synthetic peptide"
YPGTGG

SEQ ID NO: 27 - protein, 117 aa, synthetic construct
  REGION 1..117: note = source = /note="Description of Artificial Sequence: Synthetic polypeptide"
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSS

SEQ ID NO: 28 - DNA, 351 bp, synthetic construct
  misc_feature 1..351: note = source = /note="Description of Artificial Sequence: Synthetic polynucleotide"
  source 1..351: mol_type = other DNA
gaggtgcagctggtgcagtcaggcgccgaagtgaagaagcccggcgagtcactgagaattagctgtaaaggttcaggctacaccttcactacctactggatgcactgggtccgccaggctaccggtcaaggcctcgagtggatgggtaatatctaccccggcaccggcggctctaacttcgacgagaagtttaagaatagagtgactatcaccgccgataagtctactagcaccgcctatatggaactgtctagcctgagatcagaggacaccgccgtctactactgcactaggtggactaccggcacaggcgcctactggggtcaaggcactaccgtgaccgtgtctagc

SEQ ID NO: 29 - protein, 443 aa, synthetic construct
  REGION 1..443: note = source = /note="Description of Artificial Sequence: Synthetic polypeptide"
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 30 - DNA, 1329 bp, synthetic construct
  misc_feature 1..1329: note = source = /note="Description of Artificial Sequence: Synthetic polynucleotide"
  source 1..1329: mol_type = other DNA
gaggtgcagctggtgcagtcaggcgccgaagtgaagaagcccggcgagtcactgagaattagctgtaaaggttcaggctacaccttcactacctactggatgcactgggtccgccaggctaccggtcaaggcctcgagtggatgggtaatatctaccccggcaccggcggctctaacttcgacgagaagtttaagaatagagtgactatcaccgccgataagtctactagcaccgcctatatggaactgtctagcctgagatcagaggacaccgccgtctactactgcactaggtggactaccggcacaggcgcctactggggtcaaggcactaccgtgaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 31 - protein, 17 aa, synthetic construct
  REGION 1..17: note = source = /note="Description of Artificial Sequence: Synthetic peptide"
KSSQSLLDSGNQKNFLT

SEQ ID NO: 32 - protein, 7 aa, synthetic construct
  REGION 1..7: note = source = /note="Description of Artificial Sequence: Synthetic peptide"
WASTRES

SEQ ID NO: 33 - protein, 22 aa, synthetic construct
  REGION 1..22: note = source = /note="Description of Artificial Sequence: Synthetic peptide"
YGRKKRRQRRRLYRSPAMPENL

SEQ ID NO: 34 - protein, 13 aa, synthetic construct
  REGION 1..13: note = source = /note="Description of Artificial Sequence: Synthetic peptide"
SQSLLDSGNQKNF

SEQ ID NO: 35 - skipped

SEQ ID NO: 36 - protein, 6 aa, synthetic construct
  REGION 1..6: note = source = /note="Description of Artificial Sequence: Synthetic peptide"
DYSYPY

SEQ ID NO: 37 - protein, 113 aa, synthetic construct
  REGION 1..113: note = source = /note="Description of Artificial Sequence: Synthetic polypeptide"
EIVLTQSPATLSLSPGERATLSCKSSQSLLDSGNQKNFLTWYQQKPGKAPKLLIYWASTRESGVPSRFSGSGSGTDFTFTISSLQPEDIATYYCQNDYSYPYTFGQGTKVEIK

SEQ ID NO: 38 - DNA, 339 bp, synthetic construct
  misc_feature 1..339: note = source = /note="Description of Artificial Sequence: Synthetic polynucleotide"
  source 1..339: mol_type = other DNA
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtaaagcccctaagctgctgatctactgggcctctactagagaatcaggcgtgccctctaggtttagcggtagcggtagtggcaccgacttcaccttcactatctctagcctgcagcccgaggatatcgctacctactactgtcagaacgactatagctacccctacaccttcggtcaaggcactaaggtcgagattaag

SEQ ID NO: 39 - protein, 220 aa, synthetic construct
  REGION 1..220: note = source = /note="Description of Artificial Sequence: Synthetic polypeptide"
EIVLTQSPATLSLSPGERATLSCKSSQSLLDSGNQKNFLTWYQQKPGKAPKLLIYWASTRESGVPSRFSGSGSGTDFTFTISSLQPEDIATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - DNA, 660 bp, synthetic construct
  misc_feature 1..660: note = source = /note="Description of Artificial Sequence: Synthetic polynucleotide"
  source 1..660: mol_type = other DNA
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtaaagcccctaagctgctgatctactgggcctctactagagaatcaggcgtgccctctaggtttagcggtagcggtagtggcaccgacttcaccttcactatctctagcctgcagcccgaggatatcgctacctactactgtcagaacgactatagctacccctacaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 41 - protein, 113 aa, synthetic construct
  REGION 1..113: note = source = /note="Description of Artificial Sequence: Synthetic polypeptide"
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIK

SEQ ID NO: 42 - DNA, 339 bp, synthetic construct
  misc_feature 1..339: note = source = /note="Description of Artificial Sequence: Synthetic polynucleotide"
  source 1..339: mol_type = other DNA
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtcaagcccctagactgctgatctactgggcctctactagagaatcaggcgtgccctctaggtttagcggtagcggtagtggcaccgacttcaccttcactatctctagcctggaagccgaggacgccgctacctactactgtcagaacgactatagctacccctacaccttcggtcaaggcactaaggtcgagattaag

SEQ ID NO: 43 - protein, 220 aa, synthetic construct
  REGION 1..220: note = source = /note="Description of Artificial Sequence: Synthetic polypeptide"
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - DNA, 660 bp, synthetic construct
  misc_feature 1..660: note = source = /note="Description of Artificial Sequence: Synthetic polynucleotide"
  source 1..660: mol_type = other DNA
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtcaagcccctagactgctgatctactgggcctctactagagaatcaggcgtgccctctaggtttagcggtagcggtagtggcaccgacttcaccttcactatctctagcctggaagccgaggacgccgctacctactactgtcagaacgactatagctacccctacaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 45 - DNA, 15 bp, synthetic construct
  misc_feature 1..15: note = source = /note="Description of Artificial Sequence: Synthetic oligonucleotide"
  source 1..15: mol_type = other DNA
acctactggatgcac

SEQ ID NO: 46 - DNA, 51 bp, synthetic construct
  misc_feature 1..51: note = source = /note="Description of Artificial Sequence: Synthetic oligonucleotide"
  source 1..51: mol_type = other DNA
aatatctaccccggcaccggcggctctaacttcgacgagaagtttaagaat

SEQ ID NO: 47 - DNA, 24 bp, synthetic construct
  misc_feature 1..24: note = source = /note="Description of Artificial Sequence: Synthetic oligonucleotide"
  source 1..24: mol_type = other DNA
tggactaccggcacaggcgcctac

SEQ ID NO: 48 - DNA, 21 bp, synthetic construct
  misc_feature 1..21: note = source = /note="Description of Artificial Sequence: Synthetic oligonucleotide"
  source 1..21: mol_type = other DNA
ggctacaccttcactacctac

SEQ ID NO: 49 - DNA, 18 bp, synthetic construct
  misc_feature 1..18: note = source = /note="Description of Artificial Sequence: Synthetic oligonucleotide"
  source 1..18: mol_type = other DNA
taccccggcaccggcggc

SEQ ID NO: 50 - DNA, 51 bp, synthetic construct
  misc_feature 1..51: note = source = /note="Description of Artificial Sequence: Synthetic oligonucleotide"
  source 1..51: mol_type = other DNA
aaatctagtcagtcactgctggatagcggtaatcagaagaacttcctgacc

SEQ ID NO: 51 - DNA, 21 bp, synthetic construct
  misc_feature 1..21: note = source = /note="Description of Artificial Sequence: Synthetic oligonucleotide"
  source 1..21: mol_type = other DNA
tgggcctctactagagaatca

SEQ ID NO: 52 - DNA, 27 bp, synthetic construct
  misc_feature 1..27: note = source = /note="Description of Artificial Sequence: Synthetic oligonucleotide"
  source 1..27: mol_type = other DNA
cagaacgactatagctacccctacacc

SEQ ID NO: 53 - DNA, 39 bp, synthetic construct
  misc_feature 1..39: note = source = /note="Description of Artificial Sequence: Synthetic oligonucleotide"
  source 1..39: mol_type = other DNA
agtcagtcactgctggatagcggtaatcagaagaacttc

SEQ ID NO: 54 - skipped

SEQ ID NO: 55 - DNA, 18 bp, synthetic construct
  misc_feature 1..18: note = source = /note="Description of Artificial Sequence: Synthetic oligonucleotide"
  source 1..18: mol_type = other DNA
gactatagctacccctac

SEQ ID NO: 56 - protein, 440 aa, synthetic construct
  REGION 1..440: note = source = /note="Description of Artificial Sequence: Synthetic polypeptide"
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 57 - protein, 214 aa, synthetic construct
  REGION 1..214: note = source = /note="Description of Artificial Sequence: Synthetic polypeptide"
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 447 aa, synthetic construct
  REGION 1..447: note = source = /note="Description of Artificial Sequence: Synthetic polypeptide"
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 59 - protein, 218 aa, synthetic construct
  REGION 1..218: note = source = /note="Description of Artificial Sequence: Synthetic polypeptide"
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 447 aa, synthetic construct
  REGION 1..447: note = source = /note="Description of Artificial Sequence: Synthetic polypeptide"
QVQLVQSGSELKKPGASVKISCKASGYTFTNYGMNWVRQAPGQGLQWMGWINTDSGESTYAEEFKGRFVFSLDTSVNTAYLQITSLTAEDTGMYFCVRVGYDALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 213 aa, synthetic construct
  REGION 1..213: note = source = /note="Description of Artificial Sequence: Synthetic polypeptide"
EIVLTQSPSSLSASVGDRVTITCSARSSVSYMHWFQQKPGKAPKLWIYRTSNLASGVPSRFSGSGSGTSYCLTINSLQPEDFATYYCQQRSSFPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 5 aa, synthetic construct
  REGION 1..5: note = source = /note="Description of Artificial Sequence: Synthetic peptide"
SYWMY

SEQ ID NO: 63 - protein, 17 aa, synthetic construct
  REGION 1..17: note = source = /note="Description of Artificial Sequence: Synthetic peptide"
RIDPNSGSTKYNEKFKN

SEQ ID NO: 64 - protein, 11 aa, synthetic construct
  REGION 1..11: note = source = /note="Description of Artificial Sequence: Synthetic peptide"
DYRKGLYAMDY

SEQ ID NO: 65 - protein, 7 aa, synthetic construct
  REGION 1..7: note = source = /note="Description of Artificial Sequence: Synthetic peptide"
GYTFTSY

SEQ ID NO: 66 - protein, 6 aa, synthetic construct
  REGION 1..6: note = source = /note="Description of Artificial Sequence: Synthetic peptide"
DPNSGS

SEQ ID NO: 67 - protein, 120 aa, synthetic construct
  REGION 1..120: note = source = /note="Description of Artificial Sequence: Synthetic polypeptide"
EVQLVQSGAEVKKPGATVKISCKVSGYTFTSYWMYWVRQARGQRLEWIGRIDPNSGSTKYNEKFKNRFTISRDNSKNTLYLQMNSLRAEDTAVYYCARDYRKGLYAMDYWGQGTTVTVSS

SEQ ID NO: 68 - DNA, 360 bp, synthetic construct
  misc_feature 1..360: note = source = /note="Description of Artificial Sequence: Synthetic polynucleotide"
  source 1..360: mol_type = other DNA
gaagtgcagctggtgcagtcaggcgccgaagtgaagaaacccggcgctaccgtgaagattagctgtaaagtctcaggctacaccttcactagctactggatgtactgggtccgacaggctagagggcaaagactggagtggatcggtagaatcgaccctaatagcggctctactaagtataacgagaagtttaagaataggttcactattagtagggataactctaagaacaccctgtacctgcagatgaatagcctgagagccgaggacaccgccgtctactactgcgctagagactatagaaagggcctgtacgctatggactactggggtcaaggcactaccgtgaccgtgtcttca

SEQ ID NO: 69 - protein, 446 aa, synthetic construct
  REGION 1..446: note = source = /note="Description of Artificial Sequence: Synthetic polypeptide"
EVQLVQSGAEVKKPGATVKISCKVSGYTFTSYWMYWVRQARGQRLEWIGRIDPNSGSTKYNEKFKNRFTISRDNSKNTLYLQMNSLRAEDTAVYYCARDYRKGLYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 70 - protein, 11 aa, synthetic construct
  REGION 1..11: note = source = /note="Description of Artificial Sequence: Synthetic peptide"
KASQDVGTAVA

SEQ ID NO: 71 - protein, 7 aa, synthetic construct
  REGION 1..7: note = source = /note="Description of Artificial Sequence: Synthetic peptide"
WASTRHT

SEQ ID NO: 72 - protein, 9 aa, synthetic construct
  REGION 1..9: note = source = /note="Description of Artificial Sequence: Synthetic peptide"
QQYNSYPLT

SEQ ID NO: 73 - protein, 7 aa, synthetic construct
  REGION 1..7: note = source = /note="Description of Artificial Sequence: Synthetic peptide"
SQDVGTA

SEQ ID NO: 74 - skipped

SEQ ID NO: 75 - protein, 6 aa, synthetic construct
  REGION 1..6: note = source = /note="Description of Artificial Sequence: Synthetic peptide"
YNSYPL

SEQ ID NO: 76 - DNA, 1338 bp, synthetic construct
  misc_feature 1..1338: note = source = /note="Description of Artificial Sequence: Synthetic polynucleotide"
  source 1..1338: mol_type = other DNA
gaagtgcagctggtgcagtcaggcgccgaagtgaagaaacccggcgctaccgtgaagattagctgtaaagtctcaggctacaccttcactagctactggatgtactgggtccgacaggctagagggcaaagactggagtggatcggtagaatcgaccctaatagcggctctactaagtataacgagaagtttaagaataggttcactattagtagggataactctaagaacaccctgtacctgcagatgaatagcctgagagccgaggacaccgccgtctactactgcgctagagactatagaaagggcctgtacgctatggactactggggtcaaggcactaccgtgaccgtgtcttcagctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 77 - protein, 107 aa, synthetic construct
  REGION 1..107: note = source = /note="Description of Artificial Sequence: Synthetic polypeptide"
AIQLTQSPSSLSASVGDRVTITCKASQDVGTAVAWYLQKPGQSPQLLIYWASTRHTGVPSRFSGSGSGTDFTFTISSLEAEDAATYYCQQYNSYPLTFGQGTKVEIK

SEQ ID NO: 78 - DNA, 321 bp, synthetic construct
  misc_feature 1..321: note = source = /note="Description of Artificial Sequence: Synthetic polynucleotide"
  source 1..321: mol_type = other DNA
gctattcagctgactcagtcacctagtagcctgagcgctagtgtgggcgatagagtgactatcacctgtaaagcctctcaggacgtgggcaccgccgtggcctggtatctgcagaagcctggtcaatcacctcagctgctgatctactgggcctctactagacacaccggcgtgccctctaggtttagcggtagcggtagtggcaccgacttcaccttcactatctcttcactggaagccgaggacgccgctacctactactgtcagcagtataatagctaccccctgaccttcggtcaaggcactaaggtcgagattaag

SEQ ID NO: 79 - protein, 214 aa, synthetic construct
  REGION 1..214: note = source = /note="Description of Artificial Sequence: Synthetic polypeptide"
AIQLTQSPSSLSASVGDRVTITCKASQDVGTAVAWYLQKPGQSPQLLIYWASTRHTGVPSRFSGSGSGTDFTFTISSLEAEDAATYYCQQYNSYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 80 - DNA, 642 bp, synthetic construct
  misc_feature 1..642: note = source = /note="Description of Artificial Sequence: Synthetic polynucleotide"
  source 1..642: mol_type = other DNA
gctattcagctgactcagtcacctagtagcctgagcgctagtgtgggcgatagagtgactatcacctgtaaagcctctcaggacgtgggcaccgccgtggcctggtatctgcagaagcctggtcaatcacctcagctgctgatctactgggcctctactagacacaccggcgtgccctctaggtttagcggtagcggtagtggcaccgacttcaccttcactatctcttcactggaagccgaggacgccgctacctactactgtcagcagtataatagctaccccctgaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 81 - protein, 120 aa, synthetic construct
  REGION 1..120: note = source = /note="Description of Artificial Sequence: Synthetic polypeptide"
EVQLVQSGAEVKKPGATVKISCKVSGYTFTSYWMYWVRQATGQGLEWMGRIDPNSGSTKYNEKFKNRVTITADKSTSTAYMELSSLRSEDTAVYYCARDYRKGLYAMDYWGQGTTVTVSS

SEQ ID NO: 82 - DNA, 360 bp, synthetic construct
  misc_feature 1..360: note = source = /note="Description of Artificial Sequence: Synthetic polynucleotide"
  source 1..360: mol_type = other DNA
gaagtgcagctggtgcagtcaggcgccgaagtgaagaaacccggcgctaccgtgaagattagctgtaaagtctcaggctacaccttcactagctactggatgtactgggtccgacaggctaccggtcaaggcctggagtggatgggtagaatcgaccctaatagcggctctactaagtataacgagaagtttaagaatagagtgactatcaccgccgataagtctactagcaccgcctatatggaactgtctagcctgagatcagaggacaccgccgtctactactgcgctagagactatagaaagggcctgtacgctatggactactggggtcaaggcactaccgtgaccgtgtcttca

SEQ ID NO: 83 - protein, 446 aa, synthetic construct
  REGION 1..446: note = source = /note="Description of Artificial Sequence: Synthetic polypeptide"
EVQLVQSGAEVKKPGATVKISCKVSGYTFTSYWMYWVRQATGQGLEWMGRIDPNSGSTKYNEKFKNRVTITADKSTSTAYMELSSLRSEDTAVYYCARDYRKGLYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 84 - DNA, 1338 bp, synthetic construct
  misc_feature 1..1338: note = source = /note="Description of Artificial Sequence: Synthetic polynucleotide"
  source 1..1338: mol_type = other DNA
gaagtgcagctggtgcagtcaggcgccgaagtgaagaaacccggcgctaccgtgaagattagctgtaaagtctcaggctacaccttcactagctactggatgtactgggtccgacaggctaccggtcaaggcctggagtggatgggtagaatcgaccctaatagcggctctactaagtataacgagaagtttaagaatagagtgactatcaccgccgataagtctactagcaccgcctatatggaactgtctagcctgagatcagaggacaccgccgtctactactgcgctagagactatagaaagggcctgtacgctatggactactggggtcaaggcactaccgtgaccgtgtcttcagctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 85 - protein, 107 aa, synthetic construct
  REGION 1..107: note = source = /note="Description of Artificial Sequence: Synthetic polypeptide"
DVVMTQSPLSLPVTLGQPASISCKASQDVGTAVAWYQQKPGQAPRLLIYWASTRHTGVPSRFSGSGSGTEFTLTISSLQPDDFATYYCQQYNSYPLTFGQGTKVEIK

SEQ ID NO: 86 - DNA, 321 bp, synthetic construct
  misc_feature 1..321: note = source = /note="Description of Artificial Sequence: Synthetic polynucleotide"
  source 1..321: mol_type = other DNA
gacgtcgtgatgactcagtcacccctgagcctgcccgtgaccctggggcagcccgcctctattagctgtaaagcctctcaggacgtgggcaccgccgtggcctggtatcagcagaagccagggcaagcccctagactgctgatctactgggcctctactagacacaccggcgtgccctctaggtttagcggtagcggtagtggcaccgagttcaccctgactatctcttcactgcagcccgacgacttcgctacctactactgtcagcagtataatagctaccccctgaccttcggtcaaggcactaaggtcgagattaag

SEQ ID NO: 87 - protein, 214 aa, synthetic construct
  REGION 1..214: note = source = /note="Description of Artificial Sequence: Synthetic polypeptide"
DVVMTQSPLSLPVTLGQPASISCKASQDVGTAVAWYQQKPGQAPRLLIYWASTRHTGVPSRFSGSGSGTEFTLTISSLQPDDFATYYCQQYNSYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 88 - DNA, 642 bp, synthetic construct
  misc_feature 1..642: note = source = /note="Description of Artificial Sequence: Synthetic polynucleotide"
  source 1..642: mol_type = other DNA
gacgtcgtgatgactcagtcacccctgagcctgcccgtgaccctggggcagcccgcctctattagctgtaaagcctctcaggacgtgggcaccgccgtggcctggtatcagcagaagccagggcaagcccctagactgctgatctactgggcctctactagacacaccggcgtgccctctaggtttagcggtagcggtagtggcaccgagttcaccctgactatctcttcactgcagcccgacgacttcgctacctactactgtcagcagtataatagctaccccctgaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 89 - DNA, 15 bp, synthetic construct
  misc_feature 1..15: note = source = /note="Description of Artificial Sequence: Synthetic oligonucleotide"
  source 1..15: mol_type = other DNA
agctactggatgtac

SEQ ID NO: 90 - DNA, 51 bp, synthetic construct
  misc_feature 1..51: note = source = /note="Description of Artificial Sequence: Synthetic oligonucleotide"
  source 1..51: mol_type = other DNA
agaatcgaccctaatagcggctctactaagtataacgagaagtttaagaat

SEQ ID NO: 91 - DNA, 33 bp, synthetic construct
  misc_feature 1..33: note = source = /note="Description of Artificial Sequence: Synthetic oligonucleotide"
  source 1..33: mol_type = other DNA
gactatagaaagggcctgtacgctatggactac

SEQ ID NO: 92 - DNA, 21 bp, synthetic construct
  misc_feature 1..21: note = source = /note="Description of Artificial Sequence: Synthetic oligonucleotide"
  source 1..21: mol_type = other DNA
ggctacaccttcactagctac

SEQ ID NO: 93 - DNA, 18 bp, synthetic construct
  misc_feature 1..18: note = source = /note="Description of Artificial Sequence: Synthetic oligonucleotide"
  source 1..18: mol_type = other DNA
gaccctaatagcggctct

SEQ ID NO: 94 - DNA, 33 bp, synthetic construct
  misc_feature 1..33: note = source = /note="Description of Artificial Sequence: Synthetic oligonucleotide"
  source 1..33: mol_type = other DNA
aaagcctctcaggacgtgggcaccgccgtggcc

SEQ ID NO: 95 - DNA, 21 bp, synthetic construct
  misc_feature 1..21: note = source = /note="Description of Artificial Sequence: Synthetic oligonucleotide"
  source 1..21: mol_type = other DNA
tgggcctctactagacacacc

SEQ ID NO: 96 - DNA, 27 bp, synthetic construct
  misc_feature 1..27: note = source = /note="Description of Artificial Sequence: Synthetic oligonucleotide"
  source 1..27: mol_type = other DNA
cagcagtataatagctaccccctgacc

SEQ ID NO: 97 - DNA, 21 bp, synthetic construct
  misc_feature 1..21: note = source = /note="Description of Artificial Sequence: Synthetic oligonucleotide"
  source 1..21: mol_type = other DNA
tctcaggacgtgggcaccgcc

SEQ ID NO: 98 - skipped

SEQ ID NO: 99 - DNA, 18 bp, synthetic construct
  misc_feature 1..18: note = source = /note="Description of Artificial Sequence: Synthetic oligonucleotide"
  source 1..18: mol_type = other DNA
tataatagctaccccctg

SEQ ID NO: 100 - protein, 448 aa, synthetic construct
  REGION 1..448: note = source = /note="Description of Artificial Sequence: Synthetic polypeptide"
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 101 - protein, 214 aa, synthetic construct
  REGION 1..214: note = source = /note="Description of Artificial Sequence: Synthetic polypeptide"
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 102 - protein, 450 aa, synthetic construct
  REGION 1..450: note = source = /note="Description of Artificial Sequence: Synthetic polypeptide"
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 103 - protein, 216 aa, synthetic construct
  REGION 1..216: note = source = /note="Description of Artificial Sequence: Synthetic polypeptide"
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 104 - protein, 451 aa, synthetic construct
  REGION 1..451: note = source = /note="Description of Artificial Sequence: Synthetic polypeptide"
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 105 - protein, 215 aa, synthetic construct
  REGION 1..215: note = source = /note="Description of Artificial Sequence: Synthetic polypeptide"
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 106 - protein, 123 aa, synthetic construct
  REGION 1..123: note = source = /note="Description of Artificial Sequence: Synthetic polypeptide"
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSS

SEQ ID NO: 107 - protein, 106 aa, synthetic construct
  REGION 1..106: note = source = /note="Description of Artificial Sequence: Synthetic polypeptide"
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIK

SEQ ID NO: 108 - protein, 5 aa, synthetic construct
  REGION 1..5: note = source = /note="Description of Artificial Sequence: Synthetic peptide"
NYGMN

SEQ ID NO: 109 - protein, 17 aa, synthetic construct
  REGION 1..17: note = source = /note="Description of Artificial Sequence: Synthetic peptide"
WINTDTGEPTYADDFKG

SEQ ID NO: 110 - protein, 16 aa, synthetic construct
  REGION 1..16: note = source = /note="Description of Artificial Sequence: Synthetic peptide"
NPPYYYGTNNAEAMDY

SEQ ID NO: 111 - protein, 7 aa, synthetic construct
  REGION 1..7: note = source = /note="Description of Artificial Sequence: Synthetic peptide"
GFTLTNY

SEQ ID NO: 112 - protein, 6 aa, synthetic construct
  REGION 1..6: note = source = /note="Description of Artificial Sequence: Synthetic peptide"
NTDTGE

SEQ ID NO: 113 - protein, 125 aa, synthetic construct
  REGION 1..125: note = source = /note="Description of Artificial Sequence: Synthetic polypeptide"
QVQLVQSGAEVKKPGASVKVSCKASGFTLTNYGMNWVRQARGQRLEWIGWINTDTGEPTYADDFKGRFVFSLDTSVSTAYLQISSLKAEDTAVYYCARNPPYYYGTNNAEAMDYWGQGTTVTVSS

SEQ ID NO: 114 - DNA, 375 bp, synthetic construct
  misc_feature 1..375: note = source = /note="Description of Artificial Sequence: Synthetic polynucleotide"
  source 1..375: mol_type = other DNA
caagtgcagctggtgcagtcgggagccgaagtgaagaagcctggagcctcggtgaaggtgtcgtgcaaggcatccggattcaccctcaccaattacgggatgaactgggtcagacaggcccggggtcaacggctggagtggatcggatggattaacaccgacaccggggagcctacctacgcggacgatttcaagggacggttcgtgttctccctcgacacctccgtgtccaccgcctacctccaaatctcctcactgaaagcggaggacaccgccgtgtactattgcgcgaggaacccgccctactactacggaaccaacaacgccgaagccatggactactggggccagggcaccactgtgactgtgtccagc

SEQ ID NO: 115 - DNA, 375 bp, synthetic construct
  misc_feature 1..375: note = source = /note="Description of Artificial Sequence: Synthetic polynucleotide"
  source 1..375: mol_type = other DNA
caggtgcagctggtgcagtctggcgccgaagtgaagaaacctggcgcctccgtgaaggtgtcctgcaaggcctctggcttcaccctgaccaactacggcatgaactgggtgcgacaggccaggggccagcggctggaatggatcggctggatcaacaccgacaccggcgagcctacctacgccgacgacttcaagggcagattcgtgttctccctggacacctccgtgtccaccgcctacctgcagatctccagcctgaaggccgaggataccgccgtgtactactgcgcccggaaccccccttactactacggcaccaacaacgccgaggccatggactattggggccagggcaccaccgtgaccgtgtcctct

SEQ ID NO: 116 - protein, 451 aa, synthetic construct
  REGION 1..451: note = source = /note="Description of Artificial Sequence: Synthetic polypeptide"
QVQLVQSGAEVKKPGASVKVSCKASGFTLTNYGMNWVRQARGQRLEWIGWINTDTGEPTYADDFKGRFVFSLDTSVSTAYLQISSLKAEDTAVYYCARNPPYYYGTNNAEAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 117 - protein, 11 aa, synthetic construct
  REGION 1..11: note = source = /note="Description of Artificial Sequence: Synthetic peptide"
SSSQDISNYLN

SEQ ID NO: 118 - protein, 7 aa, synthetic construct
  REGION 1..7: note = source = /note="Description of Artificial Sequence: Synthetic peptide"
YTSTLHL

SEQ ID NO: 119 - protein, 9 aa, synthetic construct
  REGION 1..9: note = source = /note="Description of Artificial Sequence: Synthetic peptide"
QQYYNLPWT

SEQ ID NO: 120 - protein, 7 aa, synthetic construct
  REGION 1..7: note = source = /note="Description of Artificial Sequence: Synthetic peptide"
SQDISNY

SEQ ID NO: 121 - skipped

SEQ ID NO: 122 - protein, 6 aa, synthetic construct
  REGION 1..6: note = source = /note="Description of Artificial Sequence: Synthetic peptide"
YYNLPW

SEQ ID NO: 123 - DNA, 1353 bp, synthetic construct
  misc_feature 1..1353: note = source = /note="Description of Artificial Sequence: Synthetic polynucleotide"
  source 1..1353: mol_type = other DNA
caagtgcagctggtgcagtcgggagccgaagtgaagaagcctggagcctcggtgaaggtgtcgtgcaaggcatccggattcaccctcaccaattacgggatgaactgggtcagacaggcccggggtcaacggctggagtggatcggatggattaacaccgacaccggggagcctacctacgcggacgatttcaagggacggttcgtgttctccctcgacacctccgtgtccaccgcctacctccaaatctcctcactgaaagcggaggacaccgccgtgtactattgcgcgaggaacccgccctactactacggaaccaacaacgccgaagccatggactactggggccagggcaccactgtgactgtgtccagcgcgtccactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 124 - DNA, 1353 bp, synthetic construct
  misc_feature 1..1353: note = source = /note="Description of Artificial Sequence: Synthetic polynucleotide"
  source 1..1353: mol_type = other DNA
caggtgcagctggtgcagtctggcgccgaagtgaagaaacctggcgcctccgtgaaggtgtcctgcaaggcctctggcttcaccctgaccaactacggcatgaactgggtgcgacaggccaggggccagcggctggaatggatcggctggatcaacaccgacaccggcgagcctacctacgccgacgacttcaagggcagattcgtgttctccctggacacctccgtgtccaccgcctacctgcagatctccagcctgaaggccgaggataccgccgtgtactactgcgcccggaaccccccttactactacggcaccaacaacgccgaggccatggactattggggccagggcaccaccgtgaccgtgtcctctgcttctaccaaggggcccagcgtgttccccctggccccctgctccagaagcaccagcgagagcacagccgccctgggctgcctggtgaaggactacttccccgagcccgtgaccgtgtcctggaacagcggagccctgaccagcggcgtgcacaccttccccgccgtgctgcagagcagcggcctgtacagcctgagcagcgtggtgaccgtgcccagcagcagcctgggcaccaagacctacacctgtaacgtggaccacaagcccagcaacaccaaggtggacaagagggtggagagcaagtacggcccaccctgccccccctgcccagcccccgagttcctgggcggacccagcgtgttcctgttcccccccaagcccaaggacaccctgatgatcagcagaacccccgaggtgacctgtgtggtggtggacgtgtcccaggaggaccccgaggtccagttcaactggtacgtggacggcgtggaggtgcacaacgccaagaccaagcccagagaggagcagtttaacagcacctaccgggtggtgtccgtgctgaccgtgctgcaccaggactggctgaacggcaaagagtacaagtgtaaggtctccaacaagggcctgccaagcagcatcgaaaagaccatcagcaaggccaagggccagcctagagagccccaggtctacaccctgccacccagccaagaggagatgaccaagaaccaggtgtccctgacctgtctggtgaagggcttctacccaagcgacatcgccgtggagtgggagagcaacggccagcccgagaacaactacaagaccacccccccagtgctggacagcgacggcagcttcttcctgtacagcaggctgaccgtggacaagtccagatggcaggagggcaacgtctttagctgctccgtgatgcacgaggccctgcacaaccactacacccagaagagcctgagcctgtccctgggc

SEQ ID NO: 125 - protein, 107 aa, synthetic construct
  REGION 1..107: note = source = /note="Description of Artificial Sequence: Synthetic polypeptide"
DIQMTQSPSSLSASVGDRVTITCSSSQDISNYLNWYLQKPGQSPQLLIYYTSTLHLGVPSRFSGSGSGTEFTLTISSLQPDDFATYYCQQYYNLPWTFGQGTKVEIK

SEQ ID NO: 126 - DNA, 321 bp, synthetic construct
  misc_feature 1..321: note = source = /note="Description of Artificial Sequence: Synthetic polynucleotide"
  source 1..321: mol_type = other DNA
gatattcagatgactcagtcacctagtagcctgagcgctagtgtgggcgatagagtgactatcacctgtagctctagtcaggatatctctaactacctgaactggtatctgcagaagcccggtcaatcacctcagctgctgatctactacactagcaccctgcacctgggcgtgccctctaggtttagcggtagcggtagtggcaccgagttcaccctgactatctctagcctgcagcccgacgacttcgctacctactactgtcagcagtactataacctgccctggaccttcggtcaaggcactaaggtcgagattaag

SEQ ID NO: 127 - DNA, 321 bp, synthetic construct
  misc_feature 1..321: note = source = /note="Description of Artificial Sequence: Synthetic polynucleotide"
  source 1..321: mol_type = other DNA
gacatccagatgacccagtccccctccagcctgtctgcttccgtgggcgacagagtgaccatcacctgttcctccagccaggacatctccaactacctgaactggtatctgcagaagcccggccagtcccctcagctgctgatctactacacctccaccctgcacctgggcgtgccctccagattttccggctctggctctggcaccgagtttaccctgaccatcagctccctgcagcccgacgacttcgccacctactactgccagcagtactacaacctgccctggaccttcggccagggcaccaaggtggaaatcaag

SEQ ID NO: 128 - protein, 214 aa, synthetic construct
  REGION 1..214: note = source = /note="Description of Artificial Sequence: Synthetic polypeptide"
DIQMTQSPSSLSASVGDRVTITCSSSQDISNYLNWYLQKPGQSPQLLIYYTSTLHLGVPSRFSGSGSGTEFTLTISSLQPDDFATYYCQQYYNLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 129 - DNA, 642 bp, synthetic construct
  misc_feature 1..642: note = source = /note="Description of Artificial Sequence: Synthetic polynucleotide"
  source 1..642: mol_type = other DNA
gatattcagatgactcagtcacctagtagcctgagcgctagtgtgggcgatagagtgactatcacctgtagctctagtcaggatatctctaactacctgaactggtatctgcagaagcccggtcaatcacctcagctgctgatctactacactagcaccctgcacctgggcgtgccctctaggtttagcggtagcggtagtggcaccgagttcaccctgactatctctagcctgcagcccgacgacttcgctacctactactgtcagcagtactataacctgccctggaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 130 - DNA, 642 bp, synthetic construct
  misc_feature 1..642: note = source = /note="Description of Artificial Sequence: Synthetic polynucleotide"
  source 1..642: mol_type = other DNA
gacatccagatgacccagtccccctccagcctgtctgcttccgtgggcgacagagtgaccatcacctgttcctccagccaggacatctccaactacctgaactggtatctgcagaagcccggccagtcccctcagctgctgatctactacacctccaccctgcacctgggcgtgccctccagattttccggctctggctctggcaccgagtttaccctgaccatcagctccctgcagcccgacgacttcgccacctactactgccagcagtactacaacctgccctggaccttcggccagggcaccaaggtggaaatcaagcgtacggtggccgctcccagcgtgttcatcttccccccaagcgacgagcagctgaagagcggcaccgccagcgtggtgtgtctgctgaacaacttctaccccagggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaacaggggcgagtgc

SEQ ID NO: 131 - protein, 125 aa, synthetic construct
  REGION 1..125: note = source = /note="Description of Artificial Sequence: Synthetic polypeptide"
QVQLVQSGAEVKKPGASVKVSCKASGFTLTNYGMNWVRQAPGQGLEWMGWINTDTGEPTYADDFKGRFVFSLDTSVSTAYLQISSLKAEDTAVYYCARNPPYYYGTNNAEAMDYWGQGTTVTVSS

SEQ ID NO: 132 - DNA, 375 bp, synthetic construct
  misc_feature 1..375: note = source = /note="Description of Artificial Sequence: Synthetic polynucleotide"
  source 1..375: mol_type = other DNA
caggtgcagctggtgcagtcaggcgccgaagtgaagaaacccggcgctagtgtgaaagtcagctgtaaagctagtggcttcaccctgactaactacgggatgaactgggtccgccaggccccaggtcaaggcctcgagtggatgggctggattaacaccgacaccggcgagcctacctacgccgacgactttaagggcagattcgtgtttagcctggacactagtgtgtctaccgcctacctgcagatctctagcctgaaggccgaggacaccgccgtctactactgcgctagaaaccccccctactactacggcactaacaacgccgaggctatggactactggggtcaaggcactaccgtgaccgtgtctagc

SEQ ID NO: 133 - DNA, 375 bp, synthetic construct
  misc_feature 1..375: note = source = /note="Description of Artificial Sequence: Synthetic polynucleotide"
  source 1..375: mol_type = other DNA
caggtgcagctggtgcagtctggcgccgaagtgaagaaacctggcgcctccgtgaaggtgtcctgcaaggcctctggcttcaccctgaccaactacggcatgaactgggtgcgacaggcccctggacagggcctggaatggatgggctggatcaacaccgacaccggcgagcctacctacgccgacgacttcaagggcagattcgtgttctccctggacacctccgtgtccaccgcctacctgcagatctccagcctgaaggccgaggataccgccgtgtactactgcgcccggaaccccccttactactacggcaccaacaacgccgaggccatggactattggggccagggcaccaccgtgaccgtgtcctct

SEQ ID NO: 134 - protein, 451 aa, synthetic construct
  REGION 1..451: note = source = /note="Description of Artificial Sequence: Synthetic polypeptide"
QVQLVQSGAEVKKPGASVKVSCKASGFTLTNYGMNWVRQAPGQGLEWMGWINTDTGEPTYADDFKGRFVFSLDTSVSTAYLQISSLKAEDTAVYYCARNPPYYYGTNNAEAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 135 - DNA, 1353 bp, synthetic construct
  misc_feature 1..1353: note = source = /note="Description of Artificial Sequence: Synthetic polynucleotide"
  source 1..1353: mol_type = other DNA
caggtgcagctggtgcagtcaggcgccgaagtgaagaaacccggcgctagtgtgaaagtcagctgtaaagctagtggcttcaccctgactaactacgggatgaactgggtccgccaggccccaggtcaaggcctcgagtggatgggctggattaacaccgacaccggcgagcctacctacgccgacgactttaagggcagattcgtgtttagcctggacactagtgtgtctaccgcctacctgcagatctctagcctgaaggccgaggacaccgccgtctactactgcgctagaaaccccccctactactacggcactaacaacgccgaggctatggactactggggtcaaggcactaccgtgaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 136 - DNA, 1353 bp, synthetic construct
  misc_feature 1..1353: note = source = /note="Description of Artificial Sequence: Synthetic polynucleotide"
  source 1..1353: mol_type = other DNA
caggtgcagctggtgcagtctggcgccgaagtgaagaaacctggcgcctccgtgaaggtgtcctgcaaggcctctggcttcaccctgaccaactacggcatgaactgggtgcgacaggcccctggacagggcctggaatggatgggctggatcaacaccgacaccggcgagcctacctacgccgacgacttcaagggcagattcgtgttctccctggacacctccgtgtccaccgcctacctgcagatctccagcctgaaggccgaggataccgccgtgtactactgcgcccggaaccccccttactactacggcaccaacaacgccgaggccatggactattggggccagggcaccaccgtgaccgtgtcctctgcttctaccaaggggcccagcgtgttccccctggccccctgctccagaagcaccagcgagagcacagccgccctgggctgcctggtgaaggactacttccccgagcccgtgaccgtgtcctggaacagcggagccctgaccagcggcgtgcacaccttccccgccgtgctgcagagcagcggcctgtacagcctgagcagcgtggtgaccgtgcccagcagcagcctgggcaccaagacctacacctgtaacgtggaccacaagcccagcaacaccaaggtggacaagagggtggagagcaagtacggcccaccctgccccccctgcccagcccccgagttcctgggcggacccagcgtgttcctgttcccccccaagcccaaggacaccctgatgatcagcagaacccccgaggtgacctgtgtggtggtggacgtgtcccaggaggaccccgaggtccagttcaactggtacgtggacggcgtggaggtgcacaacgccaagaccaagcccagagaggagcagtttaacagcacctaccgggtggtgtccgtgctgaccgtgctgcaccaggactggctgaacggcaaagagtacaagtgtaaggtctccaacaagggcctgccaagcagcatcgaaaagaccatcagcaaggccaagggccagcctagagagccccaggtctacaccctgccacccagccaagaggagatgaccaagaaccaggtgtccctgacctgtctggtgaagggcttctacccaagcgacatcgccgtggagtgggagagcaacggccagcccgagaacaactacaagaccacccccccagtgctggacagcgacggcagcttcttcctgtacagcaggctgaccgtggacaagtccagatggcaggagggcaacgtctttagctgctccgtgatgcacgaggccctgcacaaccactacacccagaagagcctgagcctgtccctgggc

SEQ ID NO: 137 - protein, 107 aa, synthetic construct
  REGION 1..107: note = source = /note="Description of Artificial Sequence: Synthetic polypeptide"
DIQMTQSPSSLSASVGDRVTITCSSSQDISNYLNWYQQKPGKAPKLLIYYTSTLHLGIPPRFSGSGYGTDFTLTINNIESEDAAYYFCQQYYNLPWTFGQGTKVEIK

SEQ ID NO: 138 - DNA, 321 bp, synthetic construct
  misc_feature 1..321: note = source = /note="Description of Artificial Sequence: Synthetic polynucleotide"
  source 1..321: mol_type = other DNA
gatattcagatgactcagtcacctagtagcctgagcgctagtgtgggcgatagagtgactatcacctgtagctctagtcaggatatctctaactacctgaactggtatcagcagaagcccggtaaagcccctaagctgctgatctactacactagcaccctgcacctgggaatcccccctaggtttagcggtagcggctacggcaccgacttcaccctgactattaacaatatcgagtcagaggacgccgcctactacttctgtcagcagtactataacctgccctggaccttcggtcaaggcactaaggtcgagattaag

SEQ ID NO: 139 - DNA, 321 bp, synthetic construct
  misc_feature 1..321: note = source = /note="Description of Artificial Sequence: Synthetic polynucleotide"
  source 1..321: mol_type = other DNA
gacatccagatgacccagtccccctccagcctgtctgcttccgtgggcgacagagtgaccatcacctgttcctccagccaggacatctccaactacctgaactggtatcagcagaagcccggcaaggcccccaagctgctgatctactacacctccaccctgcacctgggcatcccccctagattctccggctctggctacggcaccgacttcaccctgaccatcaacaacatcgagtccgaggacgccgcctactacttctgccagcagtactacaacctgccctggaccttcggccagggcaccaaggtggaaatcaag

SEQ ID NO: 140 - protein, 214 aa, synthetic construct
  REGION 1..214: note = source = /note="Description of Artificial Sequence: Synthetic polypeptide"
DIQMTQSPSSLSASVGDRVTITCSSSQDISNYLNWYQQKPGKAPKLLIYYTSTLHLGIPPRFSGSGYGTDFTLTINNIESEDAAYYFCQQYYNLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 141 - DNA, 642 bp, synthetic construct
  misc_feature 1..642: note = source = /note="Description of Artificial Sequence: Synthetic polynucleotide"
  source 1..642: mol_type = other DNA
gatattcagatgactcagtcacctagtagcctgagcgctagtgtgggcgatagagtgactatcacctgtagctctagtcaggatatctctaactacctgaactggtatcagcagaagcccggtaaagcccctaagctgctgatctactacactagcaccctgcacctgggaatcccccctaggtttagcggtagcggctacggcaccgacttcaccctgactattaacaatatcgagtcagaggacgccgcctactacttctgtcagcagtactataacctgccctggaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 142 - DNA, 642 bp, synthetic construct
  misc_feature 1..642: note = source = /note="Description of Artificial Sequence: Synthetic polynucleotide"
  source 1..642: mol_type = other DNA
gacatccagatgacccagtccccctccagcctgtctgcttccgtgggcgacagagtgaccatcacctgttcctccagccaggacatctccaactacctgaactggtatcagcagaagcccggcaaggcccccaagctgctgatctactacacctccaccctgcacctgggcatcccccctagattctccggctctggctacggcaccgacttcaccctgaccatcaacaacatcgagtccgaggacgccgcctactacttctgccagcagtactacaacctgccctggaccttcggccagggcaccaaggtggaaatcaagcgtacggtggccgctcccagcgtgttcatcttccccccaagcgacgagcagctgaagagcggcaccgccagcgtggtgtgtctgctgaacaacttctaccccagggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaacaggggcgagtgc

SEQ ID NO: 143 - DNA, 15 bp, synthetic construct
  misc_feature 1..15: note = source = /note="Description of Artificial Sequence: Synthetic oligonucleotide"
  source 1..15: mol_type = other DNA
aattacgggatgaac

SEQ ID NO: 144 - DNA, 15 bp, synthetic construct
  misc_feature 1..15: note = source = /note="Description of Artificial Sequence: Synthetic oligonucleotide"
  source 1..15: mol_type = other DNA
aactacggcatgaac

SEQ ID NO: 145 - DNA, 51 bp, synthetic construct
  misc_feature 1..51: note = source = /note="Description of Artificial Sequence: Synthetic oligonucleotide"
  source 1..51: mol_type = other DNA
tggattaacaccgacaccggggagcctacctacgcggacgatttcaaggga

SEQ ID NO: 146 - DNA, 51 bp, synthetic construct
  misc_feature 1..51: note = source = /note="Description of Artificial Sequence: Synthetic oligonucleotide"
  source 1..51: mol_type = other DNA
tggatcaacaccgacaccggcgagcctacctacgccgacgacttcaagggc

SEQ ID NO: 147 - DNA, 48 bp, synthetic construct
  misc_feature 1..48: note = source = /note="Description of Artificial Sequence: Synthetic oligonucleotide"
  source 1..48: mol_type = other DNA
aacccgccctactactacggaaccaacaacgccgaagccatggactac

SEQ ID NO: 148 - DNA, 48 bp, synthetic construct
  misc_feature 1..48: note = source = /note="Description of Artificial Sequence: Synthetic oligonucleotide"
  source 1..48: mol_type = other DNA
aaccccccttactactacggcaccaacaacgccgaggccatggactat

SEQ ID NO: 149 - DNA, 21 bp, synthetic construct
  misc_feature 1..21: note = source = /note="Description of Artificial Sequence: Synthetic oligonucleotide"
  source 1..21: mol_type = other DNA
ggattcaccctcaccaattac

SEQ ID NO: 150 - DNA, 21 bp, synthetic construct
  misc_feature 1..21: note = source = /note="Description of Artificial Sequence: Synthetic oligonucleotide"
  source 1..21: mol_type = other DNA
ggcttcaccctgaccaactac

SEQ ID NO: 151 - DNA, 18 bp, synthetic construct
  misc_feature 1..18: note = source = /note="Description of Artificial Sequence: Synthetic oligonucleotide"
  source 1..18: mol_type = other DNA
aacaccgacaccggggag

SEQ ID NO: 152 - DNA, 18 bp, synthetic construct
  misc_feature 1..18: note = source = /note="Description of Artificial Sequence: Synthetic oligonucleotide"
  source 1..18: mol_type = other DNA
aacaccgacaccggcgag

SEQ ID NO: 153 - DNA, 33 bp, synthetic construct
  misc_feature 1..33: note = source = /note="Description of Artificial Sequence: Synthetic oligonucleotide"
  source 1..33: mol_type = other DNA
agctctagtcaggatatctctaactacctgaac

SEQ ID NO: 154 - DNA, 33 bp, synthetic construct
  misc_feature 1..33: note = source = /note="Description of Artificial Sequence: Synthetic oligonucleotide"
  source 1..33: mol_type = other DNA
tcctccagccaggacatctccaactacctgaac

SEQ ID NO: 155 - DNA, 21 bp, synthetic construct
  misc_feature 1..21: note = source = /note="Description of Artificial Sequence: Synthetic oligonucleotide"
  source 1..21: mol_type = other DNA
tacactagcaccctgcacctg

SEQ ID NO: 156 - DNA, 21 bp, synthetic construct
  misc_feature 1..21: note = source = /note="Description of Artificial Sequence: Synthetic oligonucleotide"
  source 1..21: mol_type = other DNA
tacacctccaccctgcacctg

SEQ ID NO: 157 - DNA, 27 bp, synthetic construct
  misc_feature 1..27: note = source = /note="Description of Artificial Sequence: Synthetic oligonucleotide"
  source 1..27: mol_type = other DNA
cagcagtactataacctgccctggacc

SEQ ID NO: 158 - DNA, 27 bp, synthetic construct
  misc_feature 1..27: note = source = /note="Description of Artificial Sequence: Synthetic oligonucleotide"
  source 1..27: mol_type = other DNA
cagcagtactacaacctgccctggacc

SEQ ID NO: 159 - DNA, 21 bp, synthetic construct
  misc_feature 1..21: note = source = /note="Description of Artificial Sequence: Synthetic oligonucleotide"
  source 1..21: mol_type = other DNA
agtcaggatatctctaactac

SEQ ID NO: 160 - DNA, 21 bp, synthetic construct
  misc_feature 1..21: note = source = /note="Description of Artificial Sequence: Synthetic oligonucleotide"
  source 1..21: mol_type = other DNA
agccaggacatctccaactac

SEQ ID NO: 161-162 - skipped (2 entries)

SEQ ID NO: 163 - DNA, 18 bp, synthetic construct
  misc_feature 1..18: note = source = /note="Description of Artificial Sequence: Synthetic oligonucleotide"
  source 1..18: mol_type = other DNA
tactataacctgccctgg

SEQ ID NO: 164 - DNA, 18 bp, synthetic construct
  misc_feature 1..18: note = source = /note="Description of Artificial Sequence: Synthetic oligonucleotide"
  source 1..18: mol_type = other DNA
tactacaacctgccctgg

SEQ ID NO: 165 - DNA, 15 bp, synthetic construct
  misc_feature 1..15: note = source = /note="Description of Artificial Sequence: Synthetic oligonucleotide"
  source 1..15: mol_type = other DNA
aactacgggatgaac

SEQ ID NO: 166 - DNA, 51 bp, synthetic construct
  misc_feature 1..51: note = source = /note="Description of Artificial Sequence: Synthetic oligonucleotide"
  source 1..51: mol_type = other DNA
tggattaacaccgacaccggcgagcctacctacgccgacgactttaagggc

SEQ ID NO: 167 - DNA, 48 bp, synthetic construct
  misc_feature 1..48: note = source = /note="Description of Artificial Sequence: Synthetic oligonucleotide"
  source 1..48: mol_type = other DNA
aaccccccctactactacggcactaacaacgccgaggctatggactac

SEQ ID NO: 168 - DNA, 21 bp, synthetic construct
  misc_feature 1..21: note = source = /note="Description of Artificial Sequence: Synthetic oligonucleotide"
  source 1..21: mol_type = other DNA
ggcttcaccctgactaactac

SEQ ID NO: 169 - protein, 447 aa, synthetic construct
  REGION 1..447: note = source = /note="Description of Artificial Sequence: Synthetic polypeptide"
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 170 - protein, 214 aa, synthetic construct
  REGION 1..214: note = source = /note="Description of Artificial Sequence: Synthetic polypeptide"
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 171 - protein, 446 aa, synthetic construct
  REGION 1..446: note = source = /note="Description of Artificial Sequence: Synthetic polypeptide"
QVQLKESGPGLVAPSQSLSITCTVSGFSLTAYGVNWVRQPPGKGLEWLGMIWDDGSTDYNSALKSRLSISKDNSKSQVFLKMNSLQTDDTARYYCAREGDVAFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 172 - protein, 220 aa, synthetic construct
  REGION 1..220: note = source = /note="Description of Artificial Sequence: Synthetic polypeptide"
DIVMTQSPSSLAVSVGQKVTMSCKSSQSLLNGSNQKNYLAWYQQKPGQSPKLLVYFASTRDSGVPDRFIGSGSGTDFTLTISSVQAEDLADYFCLQHFGTPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 173 - protein, 10 aa, synthetic construct
  REGION 1..10: note = source = /note="Description of Artificial Sequence: Synthetic peptide"
GFTLTNYGMN

SEQ ID NO: 174 - protein, 5 aa, synthetic construct
  REGION 1..5: note = source = /note="Description of Artificial Sequence: Synthetic peptide"
SYNMH

SEQ ID NO: 175 - protein, 17 aa, synthetic construct
  REGION 1..17: note = source = /note="Description of Artificial Sequence: Synthetic peptide"
DIYPGNGDTSYNQKFKG

SEQ ID NO: 176 - protein, 9 aa, synthetic construct
  REGION 1..9: note = source = /note="Description of Artificial Sequence: Synthetic peptide"
VGGAFPMDY

SEQ ID NO: 177 - protein, 7 aa, synthetic construct
  REGION 1..7: note = source = /note="Description of Artificial Sequence: Synthetic peptide"
GYTFTSY

SEQ ID NO: 178 - protein, 6 aa, synthetic construct
  REGION 1..6: note = source = /note="Description of Artificial Sequence: Synthetic peptide"
YPGNGD

SEQ ID NO: 179 - protein, 118 aa, synthetic construct
  REGION 1..118: note = source = /note="Description of Artificial Sequence: Synthetic polypeptide"
QVQLVQSGAEVKKPGSSVKVSCKASGYTFTSYNMHWVRQAPGQGLEWMGDIYPGNGDTSYNQKFKGRVTITADKSTSTVYMELSSLRSEDTAVYYCARVGGAFPMDYWGQGTTVTVSS

SEQ ID NO: 180 - DNA, 354 bp, synthetic construct
  misc_feature 1..354: note = source = /note="Description of Artificial Sequence: Synthetic polynucleotide"
  source 1..354: mol_type = other DNA
caggtgcagctggtgcagtcaggcgccgaagtgaagaaacccggctctagcgtgaaagtttcttgtaaagctagtggctacaccttcactagctataatatgcactgggttcgccaggccccagggcaaggcctcgagtggatgggcgatatctaccccgggaacggcgacactagttataatcagaagtttaagggtagagtcactatcaccgccgataagtctactagcaccgtctatatggaactgagttccctgaggtctgaggacaccgccgtctactactgcgctagagtgggcggagccttccctatggactactggggtcaaggcactaccgtgaccgtgtctagc

SEQ ID NO: 181 - protein, 444 aa, synthetic construct
  REGION 1..444: note = source = /note="Description of Artificial Sequence: Synthetic polypeptide"
QVQLVQSGAEVKKPGSSVKVSCKASGYTFTSYNMHWVRQAPGQGLEWMGDIYPGNGDTSYNQKFKGRVTITADKSTSTVYMELSSLRSEDTAVYYCARVGGAFPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 182 - DNA, 1332 bp, synthetic construct
  misc_feature 1..1332: note = source = /note="Description of Artificial Sequence: Synthetic polynucleotide"
  source 1..1332: mol_type = other DNA
caggtgcagctggtgcagtcaggcgccgaagtgaagaaacccggctctagcgtgaaagtttcttgtaaagctagtggctacaccttcactagctataatatgcactgggttcgccaggccccagggcaaggcctcgagtggatgggcgatatctaccccgggaacggcgacactagttataatcagaagtttaagggtagagtcactatcaccgccgataagtctactagcaccgtctatatggaactgagttccctgaggtctgaggacaccgccgtctactactgcgctagagtgggcggagccttccctatggactactggggtcaaggcactaccgtgaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 183 - protein, 15 aa, synthetic construct
  REGION 1..15: note = source = /note="Description of Artificial Sequence: Synthetic peptide"
RASESVEYYGTSLMQ

SEQ ID NO: 184 - protein, 7 aa, synthetic construct
  REGION 1..7: note = source = /note="Description of Artificial Sequence: Synthetic peptide"
AASNVES

SEQ ID NO: 185 - protein, 9 aa, synthetic construct
  REGION 1..9: note = source = /note="Description of Artificial Sequence: Synthetic peptide"
QQSRKDPST

SEQ ID NO: 186 - protein, 11 aa, synthetic construct
  REGION 1..11: note = source = /note="Description of Artificial Sequence: Synthetic peptide"
SESVEYYGTSL

SEQ ID NO: 187 - skipped

SEQ ID NO: 188 - protein, 6 aa, synthetic construct
  REGION 1..6: note = source = /note="Description of Artificial Sequence: Synthetic peptide"
SRKDPS

SEQ ID NO: 189 - protein, 111 aa, synthetic construct
  REGION 1..111: note = source = /note="Description of Artificial Sequence: Synthetic polypeptide"
AIQLTQSPSSLSASVGDRVTITCRASESVEYYGTSLMQWYQQKPGKAPKLLIYAASNVESGVPSRFSGSGSGTDFTLTISSLQPEDFATYFCQQSRKDPSTFGGGTKVEIK

SEQ ID NO: 190 - DNA, 333 bp, synthetic construct
  misc_feature 1..333: note = source = /note="Description of Artificial Sequence: Synthetic polynucleotide"
  source 1..333: mol_type = other DNA
gctattcagctgactcagtcacctagtagcctgagcgctagtgtgggcgatagagtgactatcacctgtagagctagtgaatcagtcgagtactacggcactagcctgatgcagtggtatcagcagaagcccgggaaagcccctaagctgctgatctacgccgcctctaacgtggaatcaggcgtgccctctaggtttagcggtagcggtagtggcaccgacttcaccctgactatctctagcctgcagcccgaggacttcgctacctacttctgtcagcagtctaggaaggaccctagcaccttcggcggaggcactaaggtcgagattaag

SEQ ID NO: 191 - protein, 218 aa, synthetic construct
  REGION 1..218: note = source = /note="Description of Artificial Sequence: Synthetic polypeptide"
AIQLTQSPSSLSASVGDRVTITCRASESVEYYGTSLMQWYQQKPGKAPKLLIYAASNVESGVPSRFSGSGSGTDFTLTISSLQPEDFATYFCQQSRKDPSTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 192 - DNA, 654 bp, synthetic construct
  misc_feature 1..654: note = source = /note="Description of Artificial Sequence: Synthetic polynucleotide"
  source 1..654: mol_type = other DNA
gctattcagctgactcagtcacctagtagcctgagcgctagtgtgggcgatagagtgactatcacctgtagagctagtgaatcagtcgagtactacggcactagcctgatgcagtggtatcagcagaagcccgggaaagcccctaagctgctgatctacgccgcctctaacgtggaatcaggcgtgccctctaggtttagcggtagcggtagtggcaccgacttcaccctgactatctctagcctgcagcccgaggacttcgctacctacttctgtcagcagtctaggaaggaccctagcaccttcggcggaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 193 - protein, 17 aa, synthetic construct
  REGION 1..17: note = source = /note="Description of Artificial Sequence: Synthetic peptide"
DIYPGQGDTSYNQKFKG

SEQ ID NO: 194 - protein, 6 aa, synthetic construct
  REGION 1..6: note = source = /note="Description of Artificial Sequence: Synthetic peptide"
YPGQGD

SEQ ID NO: 195 - protein, 118 aa, synthetic construct
  REGION 1..118: note = source = /note="Description of Artificial Sequence: Synthetic polypeptide"
QVQLVQSGAEVKKPGASVKVSCKASGYTFTSYNMHWVRQAPGQGLEWIGDIYPGQGDTSYNQKFKGRATMTADKSTSTVYMELSSLRSEDTAVYYCARVGGAFPMDYWGQGTLVTVSS

SEQ ID NO: 196 - DNA, 354 bp, synthetic construct
  misc_feature 1..354: note = source = /note="Description of Artificial Sequence: Synthetic polynucleotide"
  source 1..354: mol_type = other DNA
caggtgcagctggtgcagtcaggcgccgaagtgaagaaacccggcgctagtgtgaaagttagctgtaaagctagtggctatactttcacttcttataatatgcactgggtccgccaggccccaggtcaaggcctcgagtggatcggcgatatctaccccggtcaaggcgacacttcctataatcagaagtttaagggtagagctactatgaccgccgataagtctacttctaccgtctatatggaactgagttccctgaggtctgaggacaccgccgtctactactgcgctagagtgggcggagccttcccaatggactactggggtcaaggcaccctggtcaccgtgtctagc

SEQ ID NO: 197 - protein, 444 aa, synthetic construct
  REGION 1..444: note = source = /note="Description of Artificial Sequence: Synthetic polypeptide"
QVQLVQSGAEVKKPGASVKVSCKASGYTFTSYNMHWVRQAPGQGLEWIGDIYPGQGDTSYNQKFKGRATMTADKSTSTVYMELSSLRSEDTAVYYCARVGGAFPMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 198 - DNA, 1332 bp, synthetic construct
  misc_feature 1..1332: note = source = /note="Description of Artificial Sequence: Synthetic polynucleotide"
  source 1..1332: mol_type = other DNA
caggtgcagctggtgcagtcaggcgccgaagtgaagaaacccggcgctagtgtgaaagttagctgtaaagctagtggctatactttcacttcttataatatgcactgggtccgccaggccccaggtcaaggcctcgagtggatcggcgatatctaccccggtcaaggcgacacttcctataatcagaagtttaagggtagagctactatgaccgccgataagtctacttctaccgtctatatggaactgagttccctgaggtctgaggacaccgccgtctactactgcgctagagtgggcggagccttcccaatggactactggggtcaaggcaccctggtcaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 199 - protein, 111 aa, synthetic construct
  REGION 1..111: note = source = /note="Description of Artificial Sequence: Synthetic polypeptide"
DIVLTQSPDSLAVSLGERATINCRASESVEYYGTSLMQWYQQKPGQPPKLLIYAASNVESGVPDRFSGSGSGTDFTLTISSLQAEDVAVYYCQQSRKDPSTFGGGTKVEIK

SEQ ID NO: 200 - DNA, 333 bp, synthetic construct
  misc_feature 1..333: note = source = /note="Description of Artificial Sequence: Synthetic polynucleotide"
  source 1..333: mol_type = other DNA
gatatcgtcctgactcagtcacccgatagcctggccgtcagcctgggcgagcgggctactattaactgtagagctagtgaatcagtcgagtactacggcactagcctgatgcagtggtatcagcagaagcccggtcaaccccctaagctgctgatctacgccgcctctaacgtggaatcaggcgtgcccgataggtttagcggtagcggtagtggcaccgacttcaccctgactattagtagcctgcaggccgaggacgtggccgtctactactgtcagcagtctaggaaggaccctagcaccttcggcggaggcactaaggtcgagattaag

SEQ ID NO: 201 - protein, 218 aa, synthetic construct
  REGION 1..218: note = source = /note="Description of Artificial Sequence: Synthetic polypeptide"
DIVLTQSPDSLAVSLGERATINCRASESVEYYGTSLMQWYQQKPGQPPKLLIYAASNVESGVPDRFSGSGSGTDFTLTISSLQAEDVAVYYCQQSRKDPSTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 202 - DNA, 654 bp, synthetic construct
  misc_feature 1..654: note = source = /note="Description of Artificial Sequence: Synthetic polynucleotide"
  source 1..654: mol_type = other DNA
gatatcgtcctgactcagtcacccgatagcctggccgtcagcctgggcgagcgggctactattaactgtagagctagtgaatcagtcgagtactacggcactagcctgatgcagtggtatcagcagaagcccggtcaaccccctaagctgctgatctacgccgcctctaacgtggaatcaggcgtgcccgataggtttagcggtagcggtagtggcaccgacttcaccctgactattagtagcctgcaggccgaggacgtggccgtctactactgtcagcagtctaggaaggaccctagcaccttcggcggaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 203 - protein, 114 aa, synthetic construct
  REGION 1..114: note = source = /note="Description of Artificial Sequence: Synthetic polypeptide"
EVQLLESGGGLVQPGGSLRLSCAAASGFTFSSYDMSWVRQAPGKGLDWVSTISGGGTYTYYQDSVKGRFTISRDNSKNTLYLQMNSLRAEDTAVYYCASMDYWGQGTTVTVSSA

SEQ ID NO: 204 - protein, 108 aa, synthetic construct
  REGION 1..108: note = source = /note="Description of Artificial Sequence: Synthetic polypeptide"
DIQMTQSPSSLSASVGDRVTITCRASQSIRRYLNWYHQKPGKAPKLLIYGASTLQSGVPSRFSGSGSGTDFTLTISSLQPEDFAVYYCQQSHSAPLTFGGGTKVEIKR

SEQ ID NO: 205 - protein, 120 aa, synthetic construct
  REGION 1..120: note = source = /note="Description of Artificial Sequence: Synthetic polypeptide"
EVQVLESGGGLVQPGGSLRLYCVASGFTFSGSYAMSWVRQAPGKGLEWVSAISGSGGSTYYADSVKGRFTISRDNSKNTLYLQMNSLRAEDTAVYYCAKKYYVGPADYWGQGTLVTVSSG

SEQ ID NO: 206 - protein, 113 aa, synthetic construct
  REGION 1..113: note = source = /note="Description of Artificial Sequence: Synthetic polypeptide"
DIVMTQSPDSLAVSLGERATINCKSSQSVLYSSNNKNYLAWYQHKPGQPPKLLIYWASTRESGVPDRFSGSGSGTDFTLTISSLQAEDVAVYYCQQYYSSPLTFGGGTKIEVK

SEQ ID NO: 207 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 208 - protein, 170 aa, Homo sapiens
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQG

SEQ ID NO: 209 - protein, 114 aa, synthetic construct
  REGION 1..114: note = source = /note="Description of Artificial Sequence: Synthetic polypeptide"
NWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 210 - protein, 297 aa, synthetic construct
  REGION 1..297: note = source = /note="Description of Artificial Sequence: Synthetic polypeptide"
ITCPPPMSVEHADIWVKSYSLYSRERYICNSGFKRKAGTSSLTECVLNKATNVAHWTTPSLKCIREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 211 - protein, 114 aa, Homo sapiens; identical to SEQ ID NO: 207
  VARIANT 93: note = /replace="Lys"
  SITE 1..114: note = /note="Variant residues given in the sequence have no preference with respect to those in the annotations for variant positions"

SEQ ID NO: 212 - protein, 77 aa, Homo sapiens
ITCPPPMSVEHADIWVKSYSLYSRERYICNSGFKRKAGTSSLTECVLNKATNVAHWTTPSLKCIRDPALVHQRPAPP

SEQ ID NO: 213 - protein, 10 aa, synthetic construct
  REGION 1..10: note = source = /note="Description of Artificial Sequence: Synthetic peptide"
GYTFTTYWMH

SEQ ID NO: 214 - protein, 10 aa, synthetic construct
  REGION 1..10: note = source = /note="Description of Artificial Sequence: Synthetic peptide"
GYTFTSYWMY

SEQ ID NO: 215 - protein, 20 aa, synthetic construct
  REGION 1..20: note = source = /note="Description of Artificial Sequence: Synthetic peptide"
GGGGSGGGGSGGGGSGGGGS

SEQ ID NO: 216 - protein, 15 aa, synthetic construct
  REGION 1..15: note = source = /note="Description of Artificial Sequence: Synthetic peptide"
GGGGSGGGGSGGGGS

SEQ ID NO: 217 - protein, 5 aa, synthetic construct
  REGION 1..5: note = source = /note="Description of Artificial Sequence: Synthetic peptide"
GGGGS

SEQ ID NO: 218 - protein, 242 aa, synthetic construct
  REGION 1..242: note = source = /note="Description of Artificial Sequence: Synthetic polypeptide"
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 219 - protein, 242 aa, synthetic construct
  REGION 1..242: note = source = /note="Description of Artificial Sequence: Synthetic polypeptide"
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSS

SEQ ID NO: 220 - protein, 242 aa, synthetic construct
  REGION 1..242: note = source = /note="Description of Artificial Sequence: Synthetic polypeptide"
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSS

SEQ ID NO: 221 - protein, 242 aa, synthetic construct
  REGION 1..242: note = source = /note="Description of Artificial Sequence: Synthetic polypeptide"
QVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 222 - protein, 242 aa, synthetic construct
  REGION 1..242: note = source = /note="Description of Artificial Sequence: Synthetic polypeptide"
QVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 223 - protein, 247 aa, synthetic construct
  REGION 1..247: note = source = /note="Description of Artificial Sequence: Synthetic polypeptide"
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSS

SEQ ID NO: 224 - protein, 247 aa, synthetic construct
  REGION 1..247: note = source = /note="Description of Artificial Sequence: Synthetic polypeptide"
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSS

SEQ ID NO: 225 - protein, 247 aa, synthetic construct
  REGION 1..247: note = source = /note="Description of Artificial Sequence: Synthetic polypeptide"
QVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 226 - protein, 247 aa, synthetic construct
  REGION 1..247: note = source = /note="Description of Artificial Sequence: Synthetic polypeptide"
QVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 227 - protein, 247 aa, synthetic construct
  REGION 1..247: note = source = /note="Description of Artificial Sequence: Synthetic polypeptide"
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSS

SEQ ID NO: 228 - protein, 247 aa, synthetic construct
  REGION 1..247: note = source = /note="Description of Artificial Sequence: Synthetic polypeptide"
QVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 229 - protein, 242 aa, synthetic construct
  REGION 1..242: note = source = /note="Description of Artificial Sequence: Synthetic polypeptide"
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSS

SEQ ID NO: 230 - protein, 242 aa, synthetic construct
  REGION 1..242: note = source = /note="Description of Artificial Sequence: Synthetic polypeptide"
QVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 231 - protein, 12 aa, synthetic construct
  REGION 1..12: note = source = /note="Description of Artificial Sequence: Synthetic peptide"
HYYYGGSYAMDY

SEQ ID NO: 232 - protein, 9 aa, synthetic construct
  REGION 1..9: note = source = /note="Description of Artificial Sequence: Synthetic peptide"
QQGNTLPYT

SEQ ID NO: 233 - protein, 18 aa, synthetic construct
  REGION 1..18: note = source = /note="Description of Artificial Sequence: Synthetic peptide"
GSTSGSGKPGSGEGSTKG

SEQ ID NO: 234 - protein, 119 aa, synthetic construct
  REGION 1..119: note = source = /note="Description of Artificial Sequence: Synthetic polypeptide"
QVQLLESGAELVRPGSSVKISCKASGYAFSSYWMNWVKQRPGQGLEWIGQIYPGDGDTNYNGKFKGQATLTADKSSSTAYMQLSGLTSEDSAVYSCARKTISSVVDFYFDYWGQGTTVT

SEQ ID NO: 235 - protein, 111 aa, synthetic construct
  REGION 1..111: note = source = /note="Description of Artificial Sequence: Synthetic polypeptide"
ELVLTQSPKFMSTSVGDRVSVTCKASQNVGTNVAWYQQKPGQSPKPLIYSATYRNSGVPDRFTGSGSGTDFTLTITNVQSKDLADYFYFCQYNRYPYTSGGGTKLEIKRRS

SEQ ID NO: 236 - protein, 248 aa, synthetic construct
  REGION 1..248: note = source = /note="Description of Artificial Sequence: Synthetic polypeptide"
QVQLLESGAELVRPGSSVKISCKASGYAFSSYWMNWVKQRPGQGLEWIGQIYPGDGDTNYNGKFKGQATLTADKSSSTAYMQLSGLTSEDSAVYSCARKTISSVVDFYFDYWGQGTTVTGSTSGSGKPGSGEGSTKGELVLTQSPKFMSTSVGDRVSVTCKASQNVGTNVAWYQQKPGQSPKPLIYSATYRNSGVPDRFTGSGSGTDFTLTITNVQSKDLADYFYFCQYNRYPYTSGGGTKLEIKRRS

SEQ ID NO: 237 - protein, 486 aa, synthetic construct
  REGION 1..486: note = source = /note="Description of Artificial Sequence: Synthetic polypeptide"
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 238 - protein, 486 aa, synthetic construct
  REGION 1..486: note = source = /note="Description of Artificial Sequence: Synthetic polypeptide"
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 239 - protein, 486 aa, synthetic construct
  REGION 1..486: note = source = /note="Description of Artificial Sequence: Synthetic polypeptide"
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 240 - protein, 486 aa, synthetic construct
  REGION 1..486: note = source = /note="Description of Artificial Sequence: Synthetic polypeptide"
MALPVTALLLPLALLLHAARPQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 241 - protein, 486 aa, synthetic construct
  REGION 1..486: note = source = /note="Description of Artificial Sequence: Synthetic polypeptide"
MALPVTALLLPLALLLHAARPQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 242 - protein, 491 aa, synthetic construct
  REGION 1..491: note = source = /note="Description of Artificial Sequence: Synthetic polypeptide"
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 243 - protein, 491 aa, synthetic construct
  REGION 1..491: note = source = /note="Description of Artificial Sequence: Synthetic polypeptide"
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 244 - protein, 491 aa, synthetic construct
  REGION 1..491: note = source = /note="Description of Artificial Sequence: Synthetic polypeptide"
MALPVTALLLPLALLLHAARPQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 245 - protein, 491 aa, synthetic construct
  REGION 1..491: note = source = /note="Description of Artificial Sequence: Synthetic polypeptide"
MALPVTALLLPLALLLHAARPQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 246 - protein, 491 aa, synthetic construct
  REGION 1..491: note = source = /note="Description of Artificial Sequence: Synthetic polypeptide"
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 247 - protein, 491 aa, synthetic construct; identical to SEQ ID NO: 246
  REGION 1..491: note = source = /note="Description of Artificial Sequence: Synthetic polypeptide"

SEQ ID NO: 248 - protein, 491 aa, synthetic construct
  REGION 1..491: note = source = /note="Description of Artificial Sequence: Synthetic polypeptide"
MALPVTALLLPLALLLHAARPQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 249 - protein, 486 aa, synthetic construct
  REGION 1..486: note = source = /note="Description of Artificial Sequence: Synthetic polypeptide"
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 250 - protein, 45 aa, synthetic construct
  REGION 1..45: note = source = /note="Description of Artificial Sequence: Synthetic polypeptide"
TTTPAPRPPTPAPTIASQPLSLRPEACRPAAGGAVHTRGLDFACD

SEQ ID NO: 251 - protein, 24 aa, synthetic construct
  REGION 1..24: note = source = /note="Description of Artificial Sequence: Synthetic peptide"
IYIWAPLAGTCGVLLLSLVITLYC

SEQ ID NO: 252 - DNA, 72 bp, synthetic construct
  misc_feature 1..72: note = source = /note="Description of Artificial Sequence: Synthetic oligonucleotide"
  source 1..72: mol_type = other DNA
atctacatctgggcgcccttggccgggacttgtggggtccttctcctgtcactggttatcaccctttactgc

SEQ ID NO: 253 - protein, 230 aa, synthetic construct
  REGION 1..230: note = source = /note="Description of Artificial Sequence: Synthetic polypeptide"
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKM

SEQ ID NO: 254 - DNA, 135 bp, synthetic construct
  misc_feature 1..135: note = source = /note="Description of Artificial Sequence: Synthetic polynucleotide"
  source 1..135: mol_type = other DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 255 - DNA, 690 bp, synthetic construct
  misc_feature 1..690: note = source = /note="Description of Artificial Sequence: Synthetic polynucleotide"
  source 1..690: mol_type = other DNA
gagagcaagtacggccctccctgccccccttgccctgcccccgagttcctgggcggacccagcgtgttcctgttcccccccaagcccaaggacaccctgatgatcagccggacccccgaggtgacctgtgtggtggtggacgtgtcccaggaggaccccgaggtccagttcaactggtacgtggacggcgtggaggtgcacaacgccaagaccaagccccgggaggagcagttcaatagcacctaccgggtggtgtccgtgctgaccgtgctgcaccaggactggctgaacggcaaggaatacaagtgtaaggtgtccaacaagggcctgcccagcagcatcgagaaaaccatcagcaaggccaagggccagcctcgggagccccaggtgtacaccctgccccctagccaagaggagatgaccaagaaccaggtgtccctgacctgcctggtgaagggcttctaccccagcgacatcgccgtggagtgggagagcaacggccagcccgagaacaactacaagaccaccccccctgtgctggacagcgacggcagcttcttcctgtacagccggctgaccgtggacaagagccggtggcaggagggcaacgtctttagctgctccgtgatgcacgaggccctgcacaaccactacacccagaagagcctgagcctgtccctgggcaagatg

SEQ ID NO: 256 - protein, 282 aa, synthetic construct
  REGION 1..282: note = source = /note="Description of Artificial Sequence: Synthetic polypeptide"
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 257 - DNA, 847 bp, synthetic construct
  misc_feature 1..847: note = source = /note="Description of Artificial Sequence: Synthetic polynucleotide"
  source 1..847: mol_type = other DNA
aggtggcccgaaagtcccaaggcccaggcatctagtgttcctactgcacagccccaggcagaaggcagcctagccaaagctactactgcacctgccactacgcgcaatactggccgtggcggggaggagaagaaaaaggagaaagagaaagaagaacaggaagagagggagaccaagacccctgaatgtccatcccatacccagccgctgggcgtctatctcttgactcccgcagtacaggacttgtggcttagagataaggccacctttacatgtttcgtcgtgggctctgacctgaaggatgcccatttgacttgggaggttgccggaaaggtacccacagggggggttgaggaagggttgctggagcgccattccaatggctctcagagccagcactcaagactcacccttccgagatccctgtggaacgccgggacctctgtcacatgtactctaaatcatcctagcctgcccccacagcgtctgatggcccttagagagccagccgcccaggcaccagttaagcttagcctgaatctgctcgccagtagtgatcccccagaggccgccagctggctcttatgcgaagtgtccggctttagcccgcccaacatcttgctcatgtggctggaggaccagcgagaagtgaacaccagcggcttcgctccagcccggcccccaccccagccgggttctaccacattctgggcctggagtgtcttaagggtcccagcaccacctagcccccagccagccacatacacctgtgttgtgtcccatgaagatagcaggaccctgctaaatgcttctaggagtctggaggtttcctacgtgactgaccatt

SEQ ID NO: 258 - protein, 10 aa, synthetic construct
  REGION 1..10: note = source = /note="Description of Artificial Sequence: Synthetic peptide"
GGGGSGGGGS

SEQ ID NO: 259 - DNA, 30 bp, synthetic construct
  misc_feature 1..30: note = source = /note="Description of Artificial Sequence: Synthetic oligonucleotide"
  source 1..30: mol_type = other DNA
ggtggcggaggttctggaggtggaggttcc

SEQ ID NO: 260 - protein, 112 aa, synthetic construct
  REGION 1..112: note = source = /note="Description of Artificial Sequence: Synthetic polypeptide"
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 261 - protein, 112 aa, synthetic construct
  REGION 1..112: note = source = /note="Description of Artificial Sequence: Synthetic polypeptide"
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 262 - DNA, 336 bp, synthetic construct
  misc_feature 1..336: note = source = /note="Description of Artificial Sequence: Synthetic polynucleotide"
  source 1..336: mol_type = other DNA
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 263 - DNA, 336 bp, synthetic construct
  misc_feature 1..336: note = source = /note="Description of Artificial Sequence: Synthetic polynucleotide"
  source 1..336: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 264 - protein, 42 aa, synthetic construct
  REGION 1..42: note = source = /note="Description of Artificial Sequence: Synthetic polypeptide"
KRGRKKLLYIFKQPFMRPVQTTQEEDGCSCRFPEEEEGGCEL

SEQ ID NO: 265 - protein, 48 aa, synthetic construct
  REGION 1..48: note = source = /note="Description of Artificial Sequence: Synthetic polypeptide"
QRRKYRSNKGESPVEPAEPCRYSCPREEEGSTIPIQEDYRKPEPACSP

SEQ ID NO: 266 - DNA, 126 bp, synthetic construct
  misc_feature 1..126: note = source = /note="Description of Artificial Sequence: Synthetic polynucleotide"
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 267 - DNA, 144 bp, synthetic construct
  misc_feature 1..144: note = source = /note="Description of Artificial Sequence: Synthetic polynucleotide"
  source 1..144: mol_type = other DNA
caacgaaggaaatatagatcaaacaaaggagaaagtcctgtggagcctgcagagccttgtcgttacagctgccccagggaggaggagggcagcaccatccccatccaggaggattaccgaaaaccggagcctgcctgctccccc

SEQ ID NO: 268 - protein, 21 aa, synthetic construct
  REGION 1..21: note = source = /note="Description of Artificial Sequence: Synthetic peptide"
MALPVTALLLPLALLLHAARP

SEQ ID NO: 269 - DNA, 2000 bp, synthetic construct
  misc_feature 1..2000: note = /note="This sequence may encompass 50-2000 nucleotides
  misc_feature 1..2000: note = source = /note="Description of Artificial Sequence: Synthetic polynucleotide"
  source 1..2000: mol_type = other DNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 270 - DNA, 1184 bp, synthetic construct
  misc_feature 1..1184: note = source = /note="Description of Artificial Sequence: Synthetic polynucleotide"
  source 1..1184: mol_type = other DNA
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 271 - protein, 448 aa, synthetic construct
  REGION 1..448: note = source = /note="Description of Artificial Sequence: Synthetic polypeptide"
QVQLQESGPGLVKPSQTLSLTCTVSDYSITSDYAWNWIRQFPGKGLEWMGYISYSGSTSYNPSLKSRITISRDTSKNQFSLQLNSVTAADTAVYYCASFDYAHAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 272 - protein, 214 aa, synthetic construct
  REGION 1..214: note = source = /note="Description of Artificial Sequence: Synthetic polypeptide"
DIVLTQSPAFLSVTPGEKVTFTCQASQSIGTSIHWYQQKTDQAPKLLIKYASESISGIPSRFSGSGSGTDFTLTISSVEAEDAADYYCQQINSWPTTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 273 - protein, 6 aa, synthetic construct
  REGION 1..6: note = source = /note="Description of Artificial Sequence: Synthetic peptide"
SDYAWN

SEQ ID NO: 274 - protein, 16 aa, synthetic construct
  REGION 1..16: note = source = /note="Description of Artificial Sequence: Synthetic peptide"
YISYSGSTSYNPSLKS

SEQ ID NO: 275 - protein, 9 aa, synthetic construct
  REGION 1..9: note = source = /note="Description of Artificial Sequence: Synthetic peptide"
FDYAHAMDY

SEQ ID NO: 276 - protein, 11 aa, synthetic construct
  REGION 1..11: note = source = /note="Description of Artificial Sequence: Synthetic peptide"
QASQSIGTSIH

SEQ ID NO: 277 - protein, 7 aa, synthetic construct
  REGION 1..7: note = source = /note="Description of Artificial Sequence: Synthetic peptide"
YASESIS

SEQ ID NO: 278 - protein, 9 aa, synthetic construct
  REGION 1..9: note = source = /note="Description of Artificial Sequence: Synthetic peptide"
QQINSWPTT

SEQ ID NO: 279 - protein, 143 aa, synthetic construct
  REGION 1..143: note = source = /note="Description of Artificial Sequence: Synthetic polypeptide"
GAPAGPLIVPYNLPLPGGVVPRMLITILGTVKPNANRIALDFQRGNDVAFHFNPRFNENNRRVIVCNTKLDNNWGREERQSVFPFESGKPFKIQVLVEPDHFKVAVNDAHLLQYNHRVKKLNEISKLGISGDIDITSASYTMI

SEQ ID NO: 280 - protein, 447 aa, synthetic construct
  REGION 1..447: note = source = /note="Description of Artificial Sequence: Synthetic polypeptide"
QVQLVQSGAEVKKPGSSVKVSCKASGYTFSSNVISWVRQAPGQGLEWMGGVIPIVDIANYAQRFKGRVTITADESTSTTYMELSSLRSEDTAVYYCASTLGLVLDAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 281 - protein, 215 aa, synthetic construct
  REGION 1..215: note = source = /note="Description of Artificial Sequence: Synthetic polypeptide"
ETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 282 - protein, 137 aa, synthetic construct
  REGION 1..137: note = source = /note="Description of Artificial Sequence: Synthetic polypeptide"
MEFGLSWVFLVALLRGVQCQVQLVESGGGVVQPGRSLRLSCAASGFTFSVYGMNWVRQAPGKGLEWVAIIWYDGDNQYYADSVKGRFTISRDNSKNTLYLQMNGLRAEDTAVYYCARDLRTGPFDYWGQGTLVTVSS

SEQ ID NO: 283 - protein, 126 aa, synthetic construct
  REGION 1..126: note = source = /note="Description of Artificial Sequence: Synthetic polypeptide"
MLPSQLIGFLLLWVPASRGEIVLTQSPDFQSVTPKEKVTITCRASQSIGSSLHWYQQKPDQSPKLLIKYASQSFSGVPSRFSGSGSGTDFTLTINSLEAEDAAAYYCHQSSSLPFTFGPGTKVDIK

SEQ ID NO: 284 - protein, 121 aa, synthetic construct
  REGION 1..121: note = source = /note="Description of Artificial Sequence: Synthetic polypeptide"
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGLWEVRALPSVYWGQGTLVTVSS

SEQ ID NO: 285 - protein, 109 aa, synthetic construct
  REGION 1..109: note = source = /note="Description of Artificial Sequence: Synthetic polypeptide"
SYELTQPPSVSVAPGQTARITCGANDIGSKSVHWYQQKAGQAPVLVVSEDIIRPSGIPERISGSNSGNTATLTISRVEAGDEADYYCQVWDRDSDQYVFGTGTKVTVLG

SEQ ID NO: 286 - protein, 9 aa, synthetic construct
  REGION 1..9: note = source = /note="Description of Artificial Sequence: Synthetic peptide"
QNDYSYPYT

SEQ ID NO: 287 - DNA, 63 bp, synthetic construct
  misc_feature 1..63: note = source = /note="Description of Artificial Sequence: Synthetic oligonucleotide"
  source 1..63: mol_type = other DNA
atggccctgcctgtgacagccctgctgctgcctctggctctgctgctgcatgccgctagaccc

SEQ ID NO: 288 - DNA, 63 bp, synthetic construct
  misc_feature 1..63: note = source = /note="Description of Artificial Sequence: Synthetic oligonucleotide"
  source 1..63: mol_type = other DNA
atggccctccctgtcaccgccctgctgcttccgctggctcttctgctccacgccgctcggccc

SEQ ID NO: 289 - DNA, 72 bp, synthetic construct
  misc_feature 1..72: note = source = /note="Description of Artificial Sequence: Synthetic oligonucleotide"
  source 1..72: mol_type = other DNA
atctacatttgggcccctctggctggtacttgcggggtcctgctgctttcactcgtgatcactctttactgt

SEQ ID NO: 290 - DNA, 126 bp, synthetic construct
  misc_feature 1..126: note = source = /note="Description of Artificial Sequence: Synthetic polynucleotide"
  source 1..126: mol_type = other DNA
aagcgcggtcggaagaagctgctgtacatctttaagcaacccttcatgaggcctgtgcagactactcaagaggaggacggctgttcatgccggttcccagaggaggaggaaggcggctgcgaactg

SEQ ID NO: 291 - DNA, 336 bp, synthetic construct
  misc_feature 1..336: note = source = /note="Description of Artificial Sequence: Synthetic polynucleotide"
  source 1..336: mol_type = other DNA
cgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 292 - protein, 5 aa, synthetic construct
  REGION 1..5: note = source = /note="Description of Artificial Sequence: Synthetic peptide"
GGGGS

SEQ ID NO: 293 - protein, 150 aa, synthetic construct [sequence omitted]

SEQ ID NO: 294 - DNA, 450 bp, synthetic construct
  misc_feature 1..450: note = source = /note="Description of Artificial Sequence: Synthetic polynucleotide"
  source 1..450: mol_type = other DNA
cccggatggtttctggactctccggatcgcccgtggaatcccccaaccttctcaccggcactcttggttgtgactgagggcgataatgcgaccttcacgtgctcgttctccaacacctccgaatcattcgtgctgaactggtaccgcatgagcccgtcaaaccagaccgacaagctcgccgcgtttccggaagatcggtcgcaaccgggacaggattgtcggttccgcgtgactcaactgccgaatggcagagacttccacatgagcgtggtccgcgctaggcgaaacgactccgggacctacctgtgcggagccatctcgctggcgcctaaggcccaaatcaaagagagcttgagggccgaactgagagtgaccgagcgcagagctgaggtgccaactgcacatccatccccatcgcctcggcctgcggggcagtttcagaccctggtc

SEQ ID NO: 295 - protein, 394 aa, synthetic construct
  REGION 1..394: note = source = /note="Description of Artificial Sequence: Synthetic polypeptide"
MALPVTALLLPLALLLHAARPPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 296 - DNA, 1182 bp, synthetic construct
  misc_feature 1..1182: note = source = /note="Description of Artificial Sequence: Synthetic polynucleotide"
  source 1..1182: mol_type = other DNA
atggccctccctgtcactgccctgcttctccccctcgcactcctgctccacgccgctagaccacccggatggtttctggactctccggatcgcccgtggaatcccccaaccttctcaccggcactcttggttgtgactgagggcgataatgcgaccttcacgtgctcgttctccaacacctccgaatcattcgtgctgaactggtaccgcatgagcccgtcaaaccagaccgacaagctcgccgcgtttccggaagatcggtcgcaaccgggacaggattgtcggttccgcgtgactcaactgccgaatggcagagacttccacatgagcgtggtccgcgctaggcgaaacgactccgggacctacctgtgcggagccatctcgctggcgcctaaggcccaaatcaaagagagcttgagggccgaactgagagtgaccgagcgcagagctgaggtgccaactgcacatccatccccatcgcctcggcctgcggggcagtttcagaccctggtcacgaccactccggcgccgcgcccaccgactccggccccaactatcgcgagccagcccctgtcgctgaggccggaagcatgccgccctgccgccggaggtgctgtgcatacccggggattggacttcgcatgcgacatctacatttgggctcctctcgccggaacttgtggcgtgctccttctgtccctggtcatcaccctgtactgcaagcggggtcggaaaaagcttctgtacattttcaagcagcccttcatgaggcccgtgcaaaccacccaggaggaggacggttgctcctgccggttccccgaagaggaagaaggaggttgcgagctgcgcgtgaagttctcccggagcgccgacgcccccgcctataagcagggccagaaccagctgtacaacgaactgaacctgggacggcgggaagagtacgatgtgctggacaagcggcgcggccgggaccccgaaatgggcgggaagcctagaagaaagaaccctcaggaaggcctgtataacgagctgcagaaggacaagatggccgaggcctactccgaaattgggatgaagggagagcggcggaggggaaaggggcacgacggcctgtaccaaggactgtccaccgccaccaaggacacatacgatgccctgcacatgcaggcccttccccctcgc

SEQ ID NO: 297 - protein, 4 aa, synthetic construct
  REGION 1..4: note = source = /note="Description of Artificial Sequence: Synthetic peptide"
GGGS

SEQ ID NO: 298 - DNA, 5000 bp, synthetic construct
  misc_feature 1..5000: note = /note="This sequence may encompass 50-5000 nucleotides
  misc_feature 1..5000: note = source = /note="Description of Artificial Sequence: Synthetic polynucleotide"
  source 1..5000: mol_type = other DNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 299 - protein, 373 aa, synthetic construct
  REGION 1..373: note = source = /note="Description of Artificial Sequence: Synthetic polypeptide"
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 300 - protein, 35 aa, synthetic construct
  REGION 1..35: note = source = /note="Description of Artificial Sequence: Synthetic polypeptide"
TKKKYSSSVHDPNGEYMFMRAVNTAKKSRLTDVTL

SEQ ID NO: 301 - DNA, 105 bp, synthetic construct
  misc_feature 1..105: note = source = /note="Description of Artificial Sequence: Synthetic polynucleotide"
  source 1..105: mol_type = other DNA
acaaaaaagaagtattcatccagtgtgcacgaccctaacggtgaatacatgttcatgagagcagtgaacacagccaaaaaatccagactcacagatgtgacccta

SEQ ID NO: 302 - protein, 69 aa, synthetic construct
  REGION 1..69: note = source = /note="Description of Artificial Sequence: Synthetic polypeptide"
TTTPAPRPPTPAPTIASQPLSLRPEACRPAAGGAVHTRGLDFACDFWLPIGCAAFVVVCILGCILICWL

SEQ ID NO: 303 - DNA, 207 bp, synthetic construct
  misc_feature 1..207: note = source = /note="Description of Artificial Sequence: Synthetic polynucleotide"
  source 1..207: mol_type = other DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttctggttacccataggatgtgcagcctttgttgtagtctgcattttgggatgcatacttatttgttggctt

SEQ ID NO: 304 - protein, 41 aa, synthetic construct
  REGION 1..41: note = source = /note="Description of Artificial Sequence: Synthetic polypeptide"
RSKRSRLLHSDYMNMTPRRPGPTRKHYQPYAPPRDFAAYRS

SEQ ID NO: 305 - DNA, 123 bp, synthetic construct
  misc_feature 1..123: note = source = /note="Description of Artificial Sequence: Synthetic polynucleotide"
  source 1..123: mol_type = other DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 306 - protein, 10 aa, synthetic construct
  REGION 1..10: note = source = /note="Description of Artificial Sequence: Synthetic peptide"
GVSLPDYGVS

SEQ ID NO: 307 - protein, 16 aa, synthetic construct
  REGION 1..16: note = source = /note="Description of Artificial Sequence: Synthetic peptide"
VIWGSETTYYNSALKS

SEQ ID NO: 308 - protein, 16 aa, synthetic construct
  REGION 1..16: note = source = /note="Description of Artificial Sequence: Synthetic peptide"
VIWGSETTYYSSSLKS

SEQ ID NO: 309 - protein, 16 aa, synthetic construct
  REGION 1..16: note = source = /note="Description of Artificial Sequence: Synthetic peptide"
VIWGSETTYYQSSLKS

SEQ ID NO: 310 - protein, 16 aa, synthetic construct
  REGION 1..16: note = source = /note="Description of Artificial Sequence: Synthetic peptide"
VIWGSETTYYNSSLKS

SEQ ID NO: 311 - protein, 11 aa, synthetic construct
  REGION 1..11: note = source = /note="Description of Artificial Sequence: Synthetic peptide"
RASQDISKYLN

SEQ ID NO: 312 - protein, 7 aa, synthetic construct
  REGION 1..7: note = source = /note="Description of Artificial Sequence: Synthetic peptide"
HTSRLHS

SEQ ID NO: 313 - protein, 40 aa, synthetic construct
  SITE 1..40: note = /note="This sequence may encompass 1-10 'Gly Gly Gly Ser' repeating units"
  REGION 1..40: note = source = /note="Description of Artificial Sequence: Synthetic polypeptide"
GGGSGGGSGGGSGGGSGGGSGGGSGGGSGGGSGGGSGGGS

SEQ ID NO: 314 - protein, 4 aa, synthetic construct
  REGION 1..4: note = source = /note="Description of Artificial Sequence: Synthetic peptide"
RGDS